(19) United States
(12) Patent Application Publication
(10) Pub. No.: US 2023/0414775 A1
(43) Pub. Date: Dec. 28, 2023

(54) TUBULYSINS AND PROTEIN-TUBULYSIN CONJUGATES

(71) Applicant: REGENERON PHARMACEUTICALS, INC., Tarrytown, NY (US)

(72) Inventor: Amy HAN, Hockessin, DE (US)

(21) Appl. No.: 18/146,343

(22) Filed: Dec. 23, 2022

Related U.S. Application Data
(60) Provisional application No. 63/294,840, filed on Dec. 29, 2021.

(51) Int. Cl. A61K 47/68; A61P 35/00; A61P 1/00; C07D 417/12

(52) U.S. Cl. CPC A61K 47/6811; A61K 47/6867; A61P 1/00; C07D 417/12; A61K 47/6869; A61K 47/6857; A61K 47/6855; A61K 47/6845; A61K 47/6849; A61K 47/6871; A61K 47/6853; A61K 47/6859; A61K 47/6873; A61K 47/6843; A61K 47/6865; A61K 47/6847; A61K 47/6863; A61P 35/00

(57) ABSTRACT

Provided herein are compounds, compositions, and methods for the treatment of diseases and disorders associated with cancer, including tubulysins and protein (e.g., antibody) drug conjugates thereof.

### CROSS-REFERENCE

This application claims the benefit under 35 U.S.C. § 119 of U.S. provisional application No. 63/294,840, filed May Dec. 29, 2021, the content of which is hereby incorporated by reference in its entirety.

### SEQUENCE LISTING

This application incorporates by reference the computer readable sequence listing in the file “114581.00582 ST26.xml,” created Mar. 15, 2023, having 28,137 bytes.

### FIELD

Provided herein are novel tubulysins and protein conjugates thereof, and methods for treating a variety of diseases, disorders, and conditions including administering the tubulysins, and protein conjugates thereof.

### BACKGROUND

While antibody-drug conjugates (ADCs) find increasing application in cancer treatment regimens, de novo or treatment-emergent resistance mechanisms could impair clinical benefit. Two resistance mechanisms that emerge under continuous ADC exposure in vitro include upregulation of transporters that confer multidrug resistance (MDR), and loss of cognate antigen expression. New technologies that circumvent these resistance mechanisms may serve to extend the utility of next generation ADCs.

The tubulysins, first isolated from myxobacterial culture broth, are a group of extremely potent tubulin polymerization inhibitors that rapidly disintegrate the cytoskeleton of dividing cells and induce apoptosis. Tubulysins are comprised of N-methyl-D-pipecolinic acid (Mep), L-isoleucine (Ile), and tubuvaline (Tuv), which contains an unusual N,O-acetal and a secondary alcohol or acetoxy group. Tubulysins A, B, C, G, and I contain the C-terminal tubutyrosine (Tut) α-amino acid, while D, E, F, and H instead have tubuphenylalanine (Tup) at this position (Angew. Chem. Int. Ed. Engl. 2004, 43, 4888-4892).

Tubulysins have emerged as promising anticancer leads due to their powerful activity in drug-resistant cells through a validated mechanism of action. The average cell growth inhibitory activity outperforms that of well-known epothilones, vinblastines, and taxols by 10-fold to more than 1000-fold, including activity against multi-drug resistant carcinoma (Biochem. J. 2006, 396, 235-242; Nat. Prod. Rep. 2015, 32, 654-662). Tubulysins have extremely potent antiproliferative activity against cancer cells, including multidrug resistant KB-V1 cervix carcinoma cells. (Angew. Chem. Int. Ed. 2004, 43, 4888-4892; and Biochemical Journal 2006, 396, 235-242).

### SUMMARY

Provided herein are compounds useful, for example, in anti-cancer and anti-angiogenesis treatments.

In one embodiment, provided are compounds having the following formula

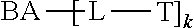

wherein
BA is a binding agent;
L is a linker covalently bound to BA and to T;
T is

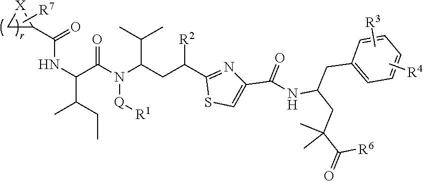

or a residue thereof covalently bound to L, wherein
X is —O— or —NR⁵;
R⁵ is hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue; or
   R⁵ is a covalent bond to L; or
   R⁵ is —(CH₂)₂—OH or —CH₂—C(O)—OH with a covalent bond to L from the terminal oxygen in —(CH₂)₂—OH or —CH₂—C(O)—OH; or
   R⁵ is —(CH₂)₂—NH₂, —(CH₂)₂—O—(CH₂)₂—NH₂, —(CH₂CH₂—O)₂—(CH₂)₂—NH₂,
   —C(O)—CH₂—NH₂, a first N-terminal amino acid residue, or a first amino acid residue with a covalent bond to L from the nitrogen in any one of —(CH₂)₂—NH₂,
   —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, —O—C(O)—NH—(CH₂CH₂O)₃—CH₂NH—C(O)CH₂NH₂, or —O—C(O)—NH—(CH₂CH₂O)₂—(CH₂)₂OH; or
   R² is —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH with a covalent bond to L from a terminal oxygen
   in any one of —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH; or
   R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
   R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or
   R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
R⁴ is hydrogen or —F;
R⁷ when present is —CH₃;
R⁶ is —OH, —NHCH₂C(O)OH, or —NH—C(O)OH; or
   R⁶ is —OH, —NHCH₂C(O)OH, or —NH—C(O)OH with a covalent bond to L from the terminal oxygen in any one of —OH, —NHCH₂C(O)OH, or —NH—C(O)OH;
Q is —CH₂— or —O—;
R¹ is —C₁-C₈ alkyl, —C₁-C₈ alkenyl, or —C₁-C₈ alkynyl;
r is three or four; and
k is an integer from one to thirty;
wherein T is not a compound selected from the table below

| ID. | Structure |
|---|---|
| P1 | 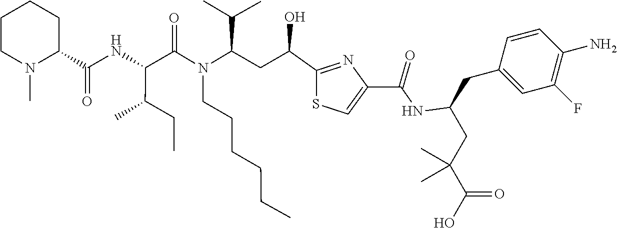 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 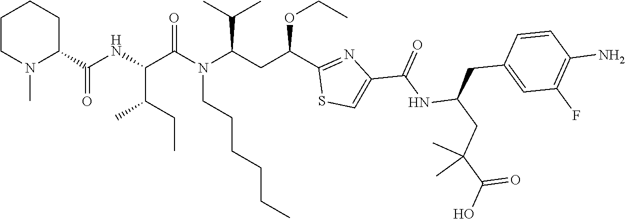 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 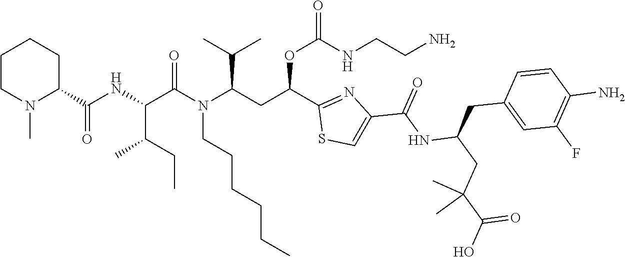 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 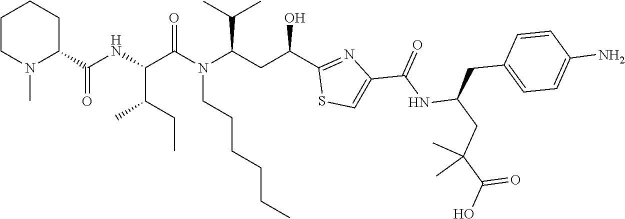 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 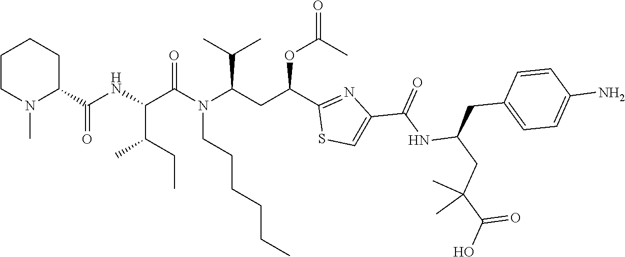 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 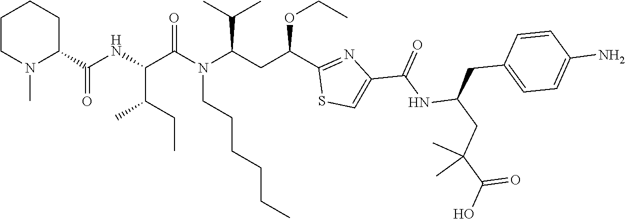 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 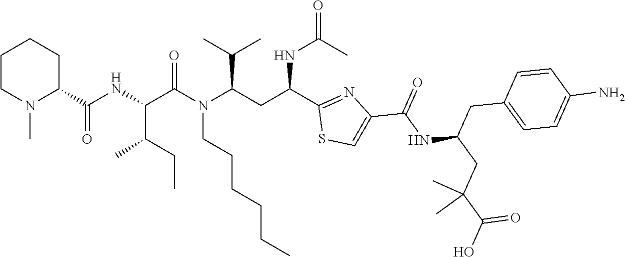 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 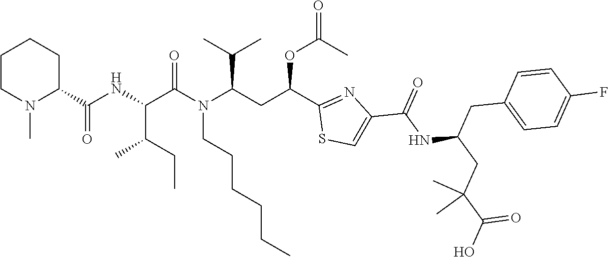 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 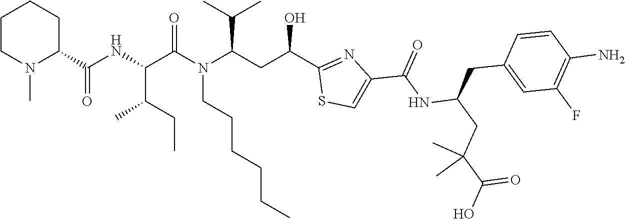 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 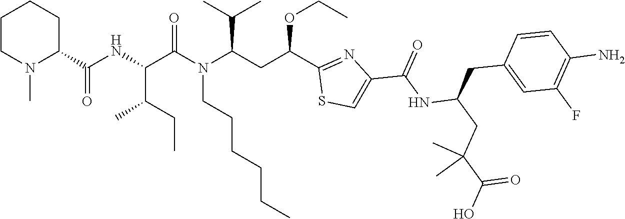 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 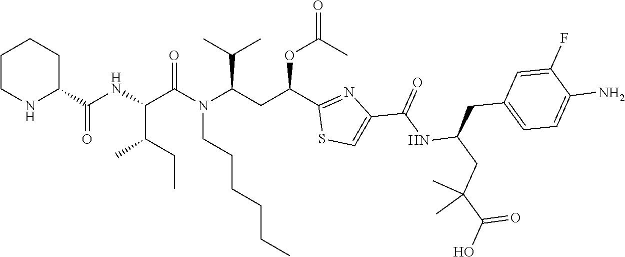 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 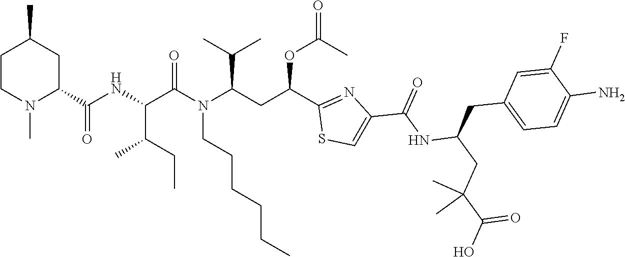 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 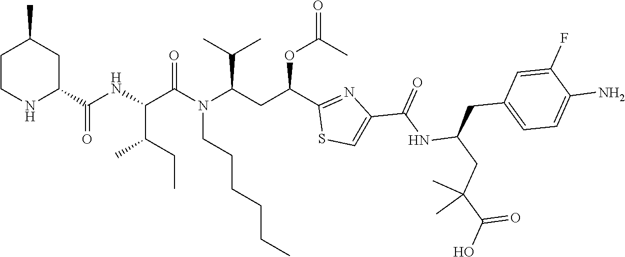 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 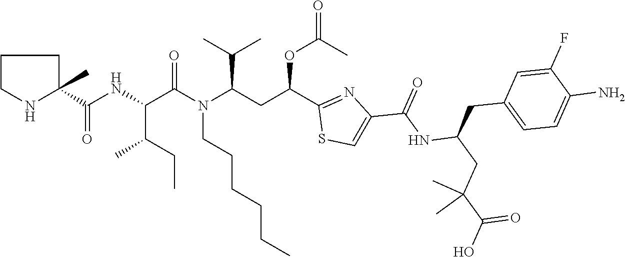 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 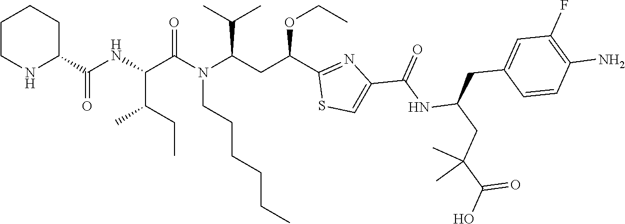 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 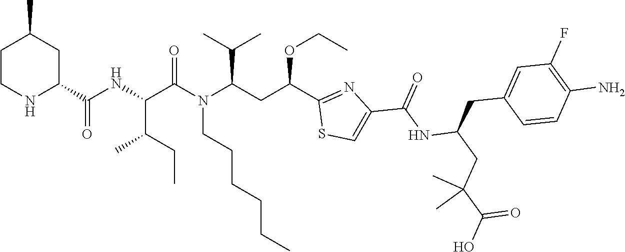 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 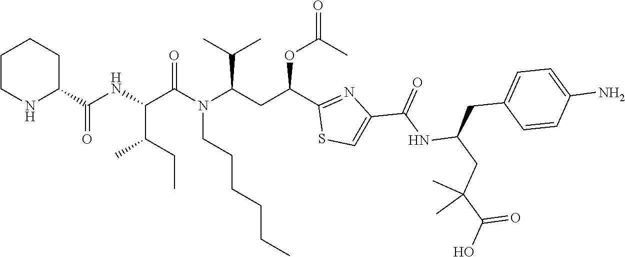 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 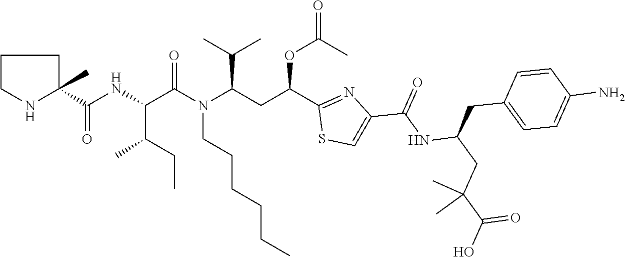 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 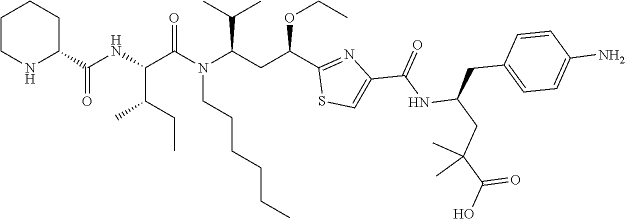 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 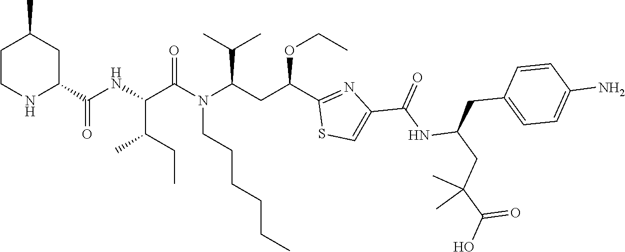 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 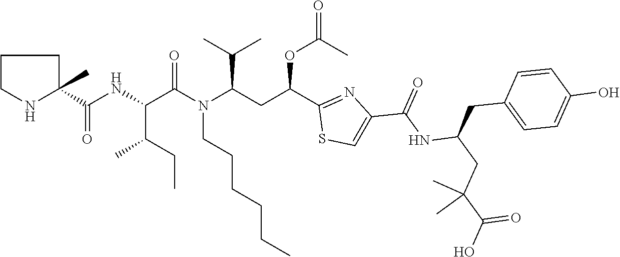 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 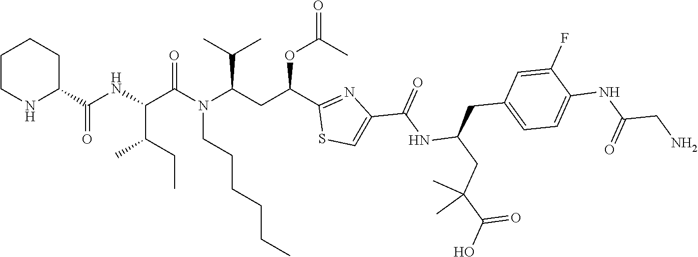 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 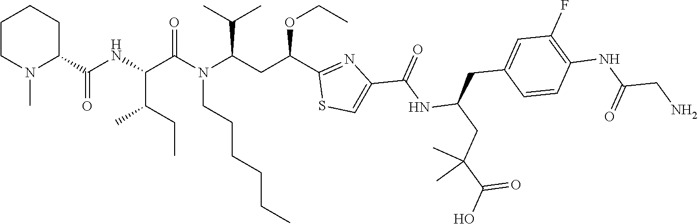 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 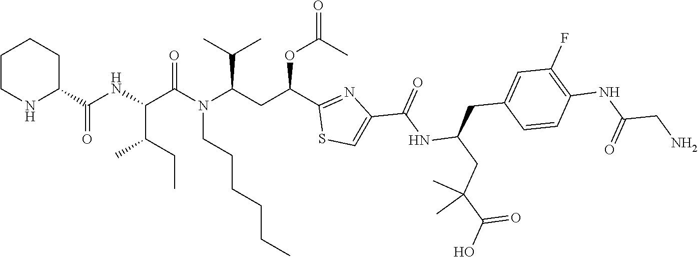 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 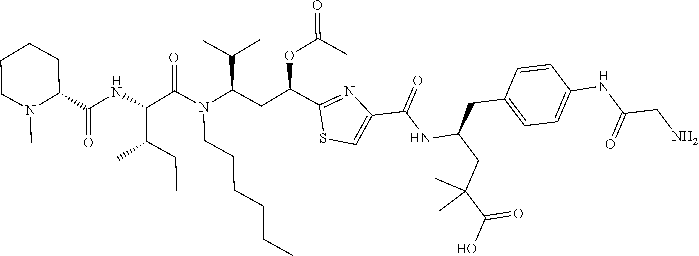 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 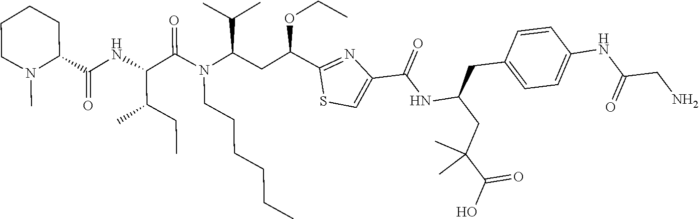 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 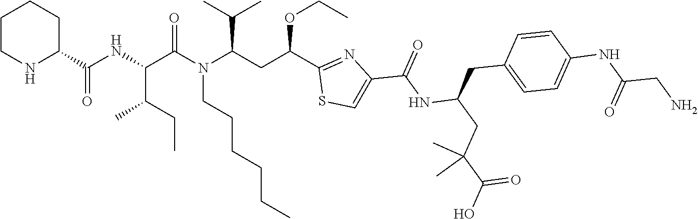 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 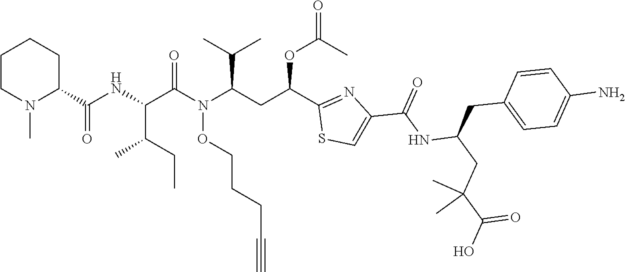 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 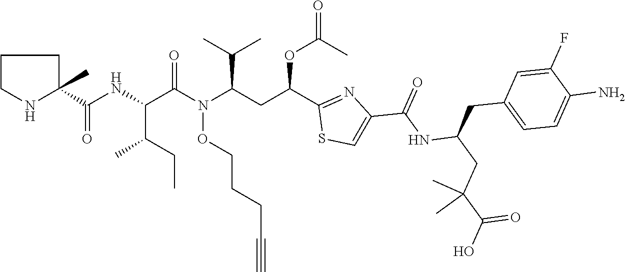 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 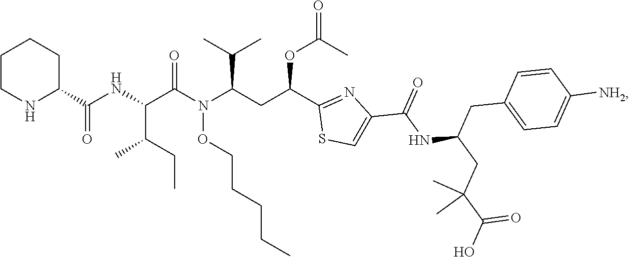 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |

and

| | |
|---|---|
| P51 | 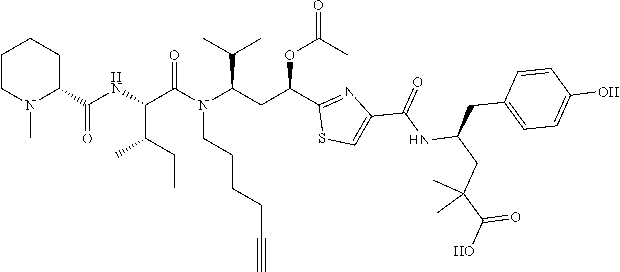 [SMILES: C#CCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 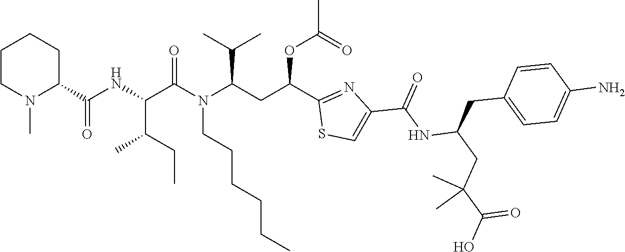 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 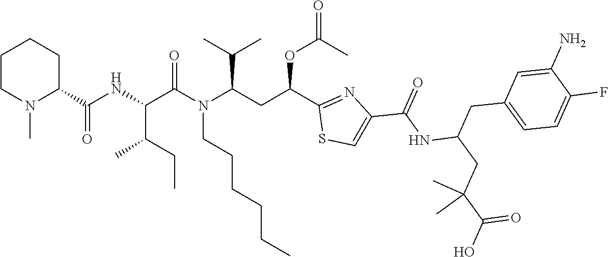 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 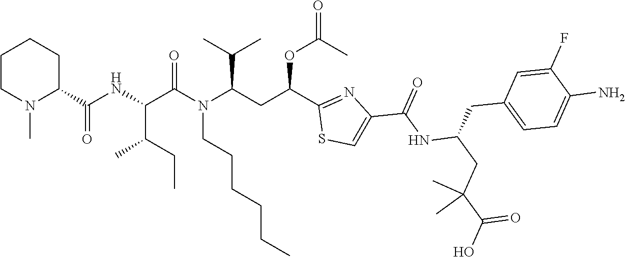 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 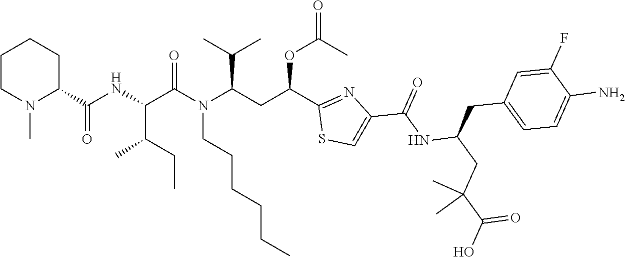 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 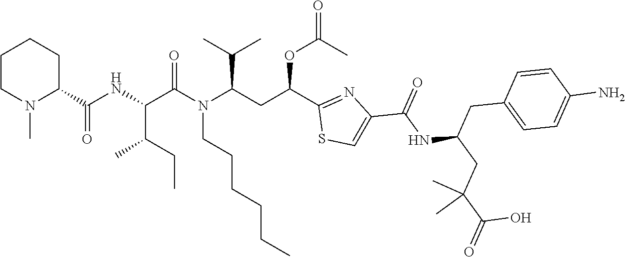 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 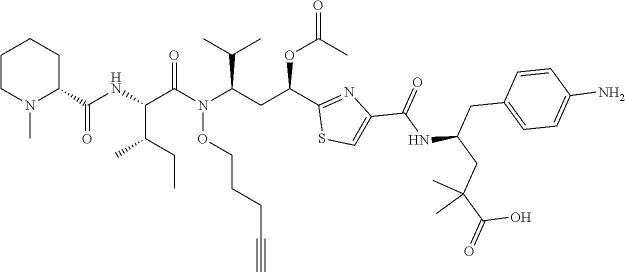 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 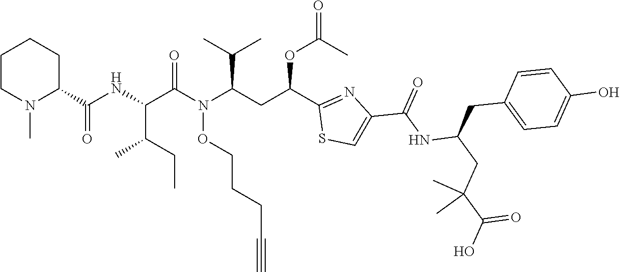 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 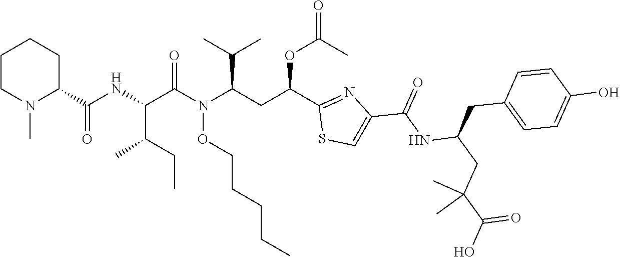 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 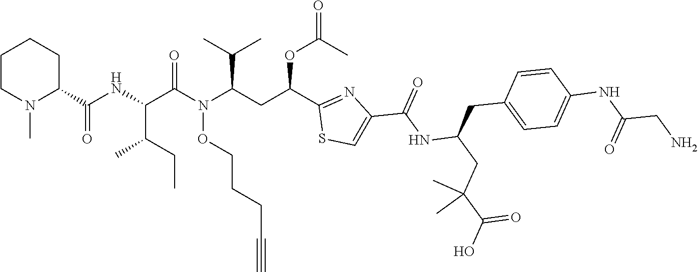 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

In one embodiment, provided are compounds having the formula

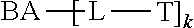

or a pharmaceutically acceptable salt thereof, wherein
BA is a binding agent;
L is a linker covalently bound to BA and to T;
T is

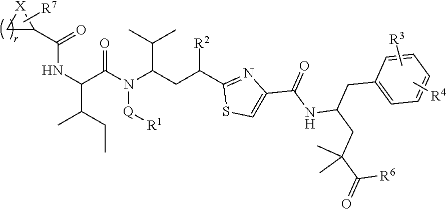

wherein
X is —O— or —NR⁵,
R⁵ is hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, or —C(O)—CH₂—NH₂;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, or —O—C(O)—NH—(CH₂CH₂O)₂—(CH₂)₂OH;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, or —NH—CH₂—C(O)—NH—(CH₂CH₂O)₂—(CH₂)₂NH;
R⁴ is hydrogen or —F;
R⁷ when present is —CH₃;
R⁶ is —OH or —NH—C(O)OH;
Q is —CH₂— or —O—,
R¹ is —C₁-C₈ alkyl, —C₁-C₈ alkenyl, or —C₁-C₈ alkynyl;
r is three or four; and
k is an integer from one to thirty;
wherein T is not a compound selected from the Table below:

| ID. | Structure |
|---|---|
| P1 | 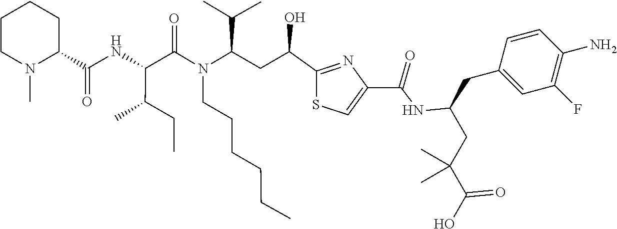 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 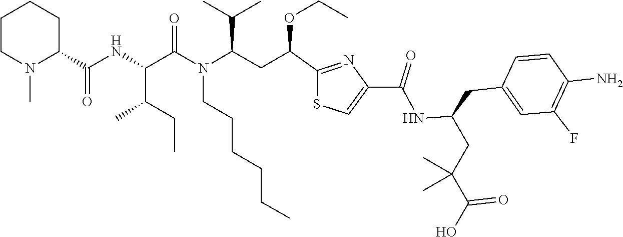 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 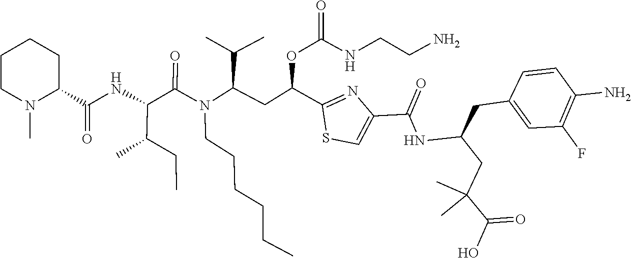 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 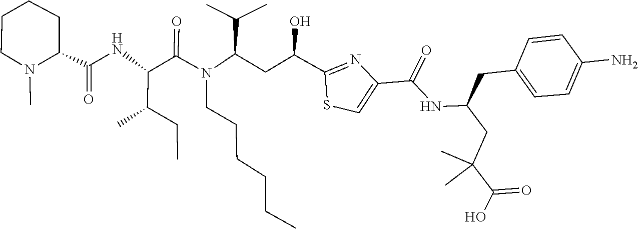 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 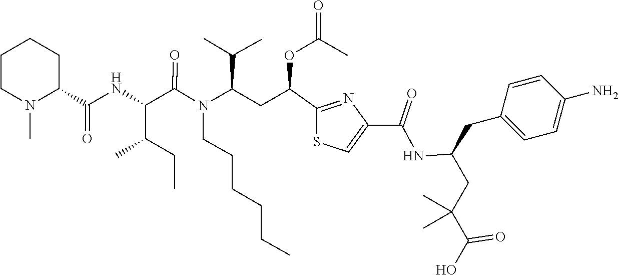 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 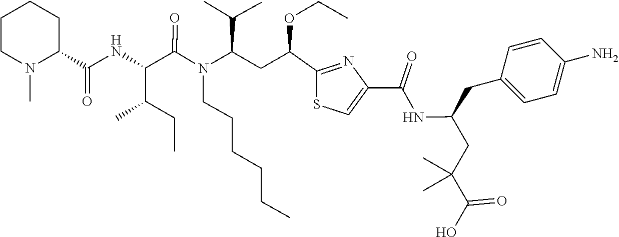 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 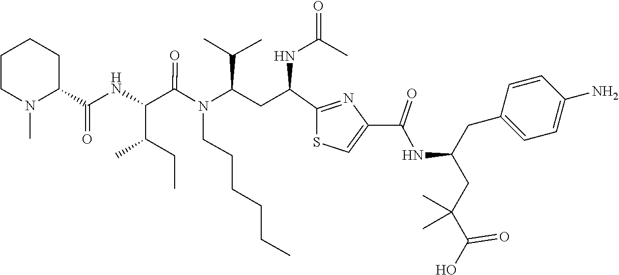 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 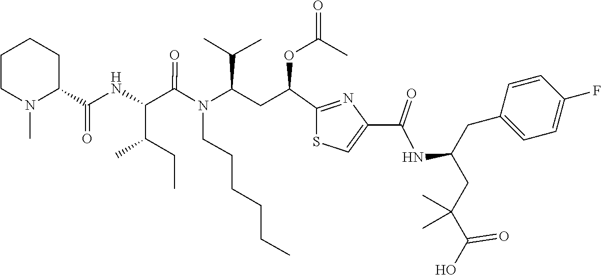 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 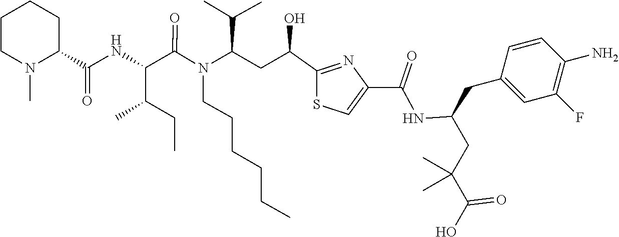 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 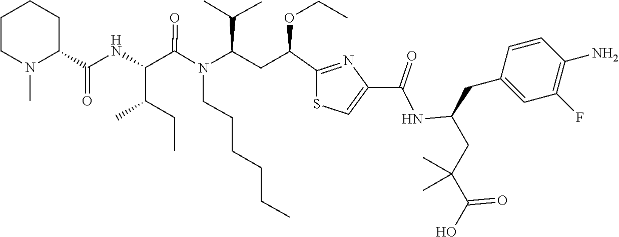 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 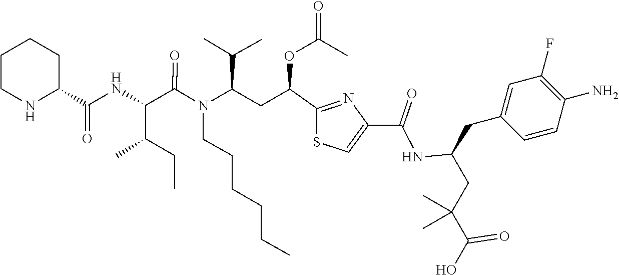 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 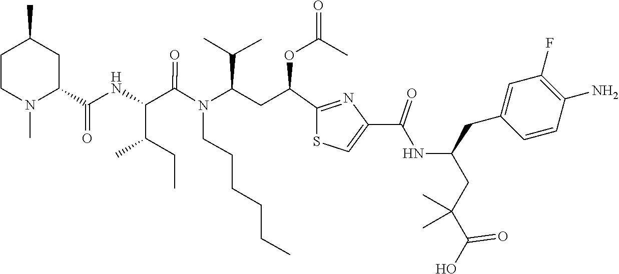 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 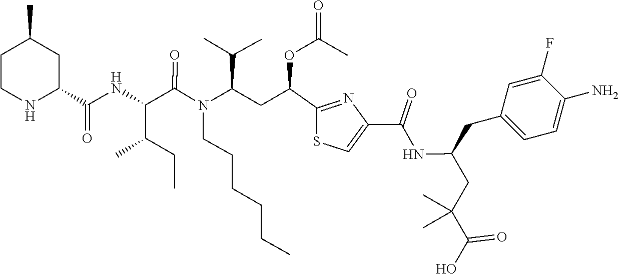 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 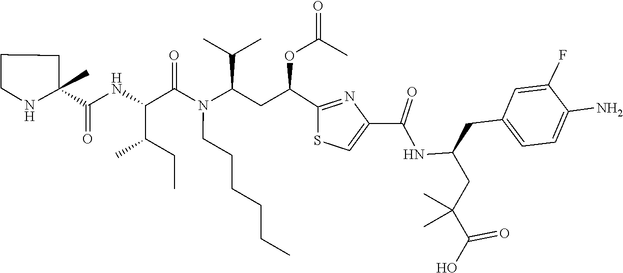 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 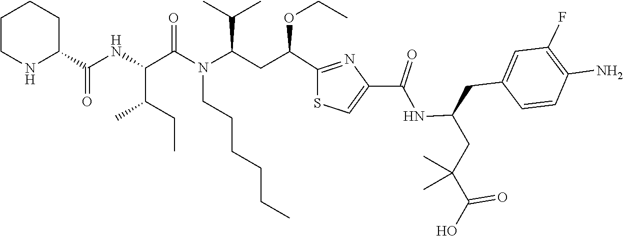 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 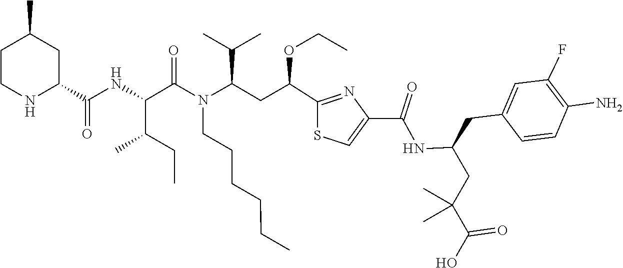 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 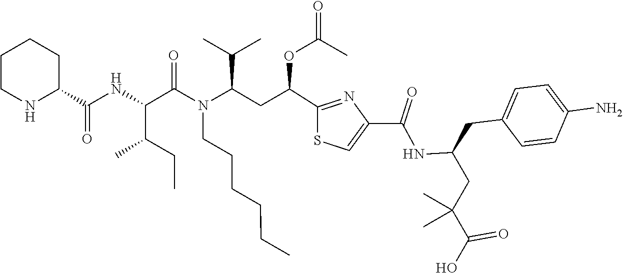 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 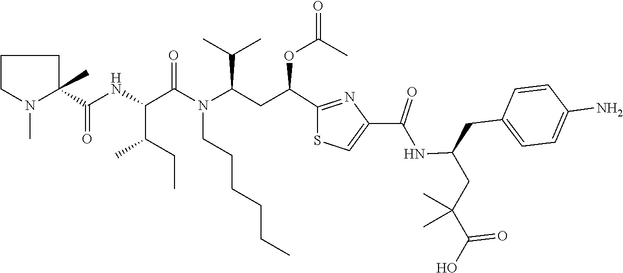 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 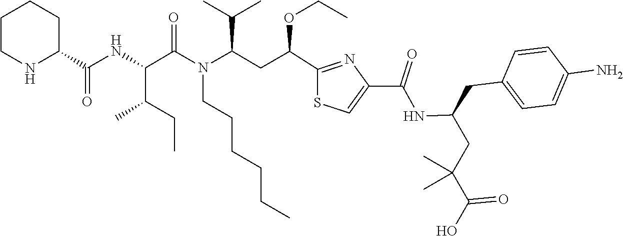 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 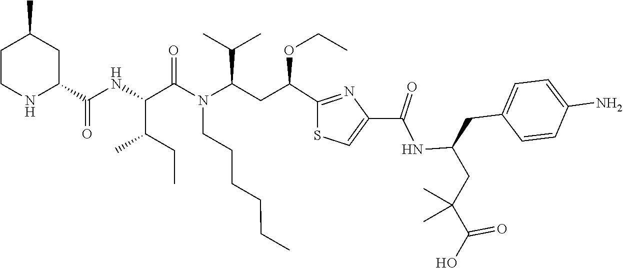 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 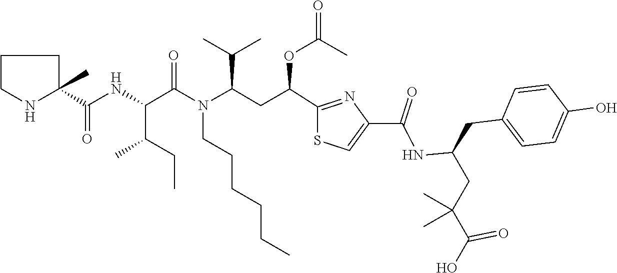 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 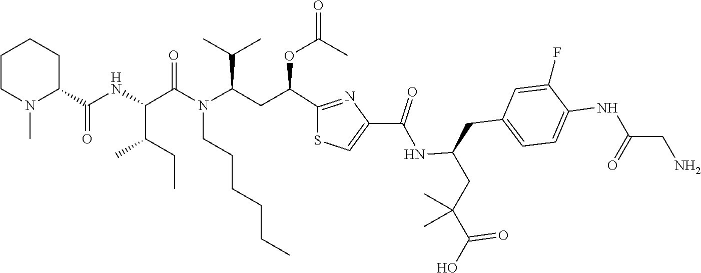 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 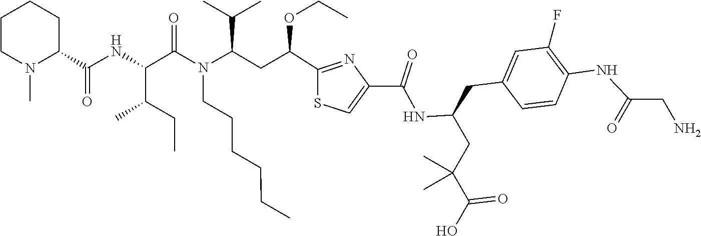 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 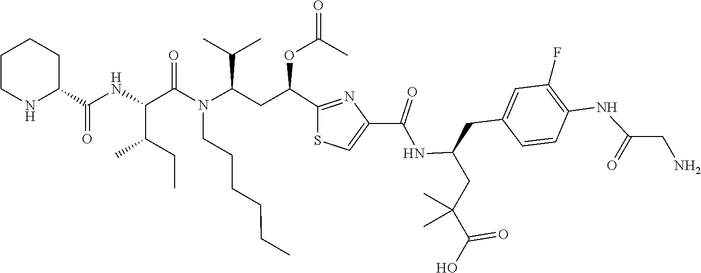 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 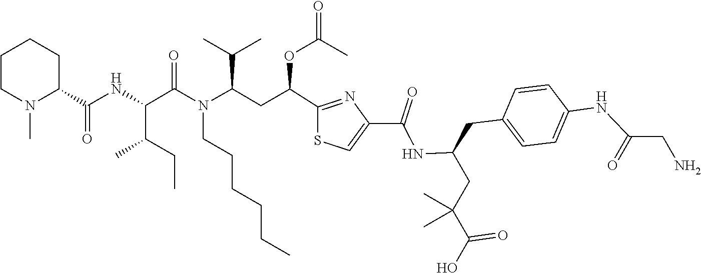 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 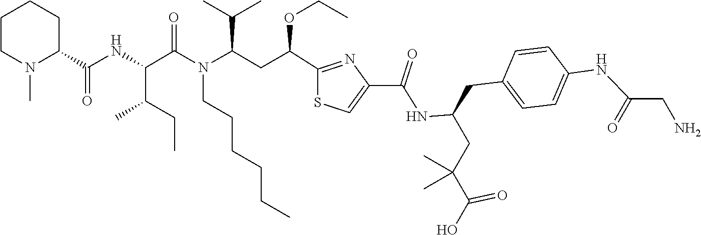 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 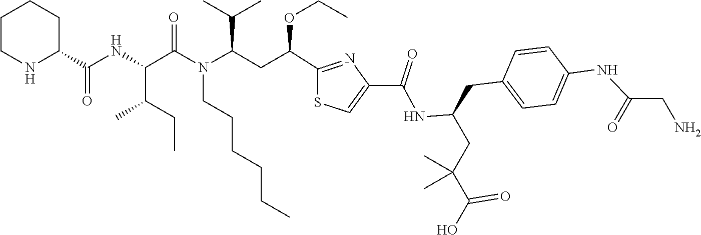 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 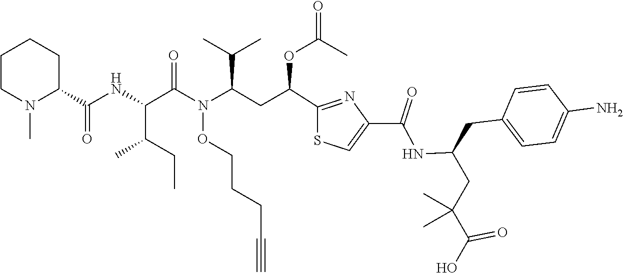 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 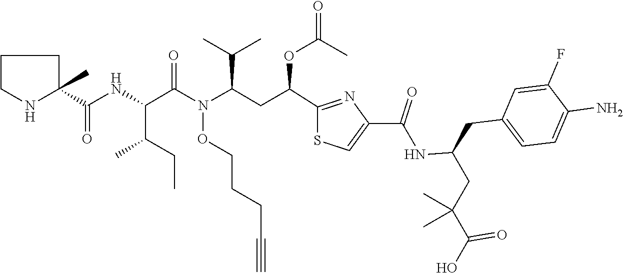 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 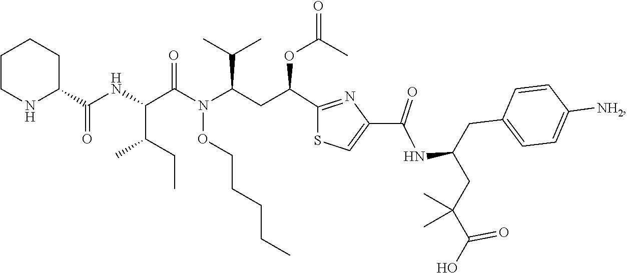 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 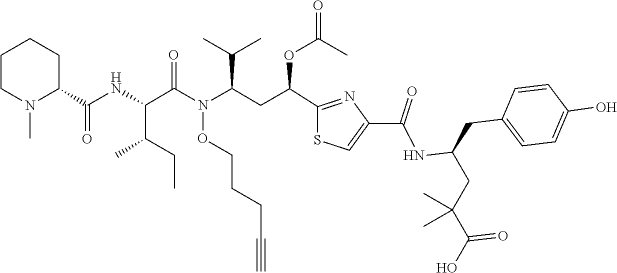 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 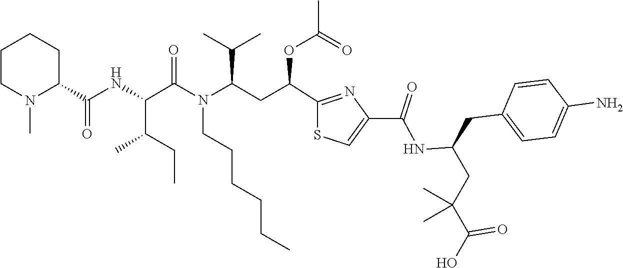 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 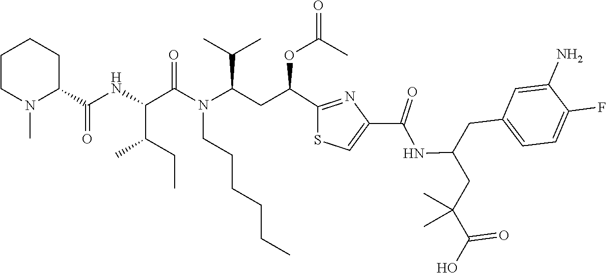 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 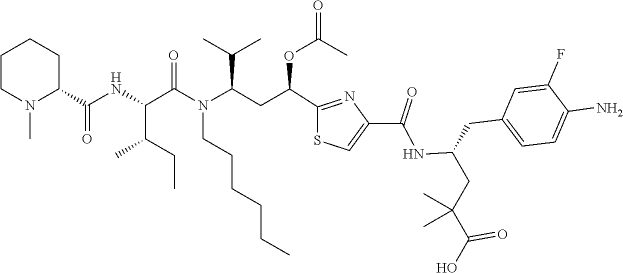 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 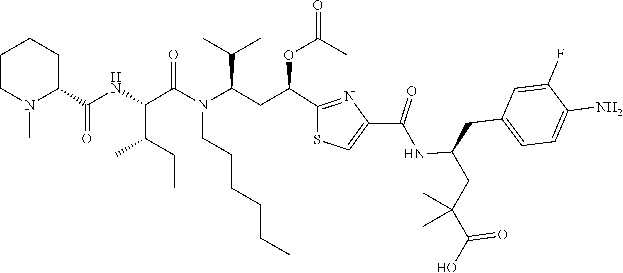 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 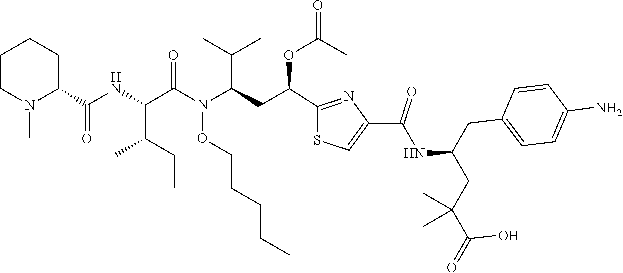 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 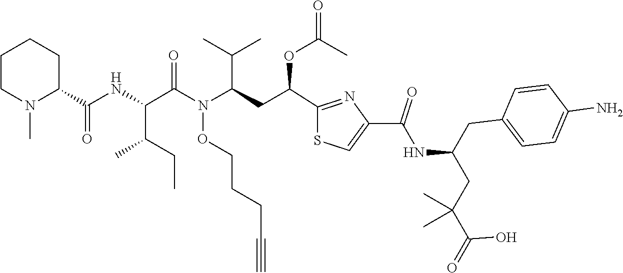 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 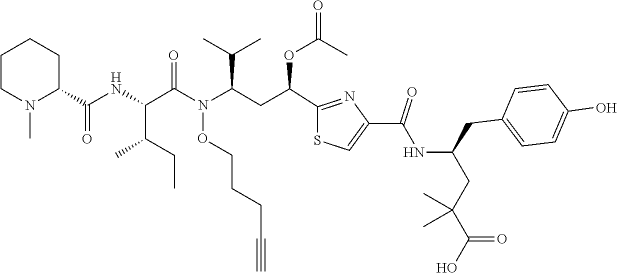 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 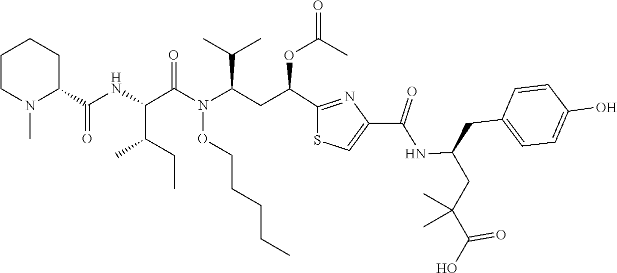 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 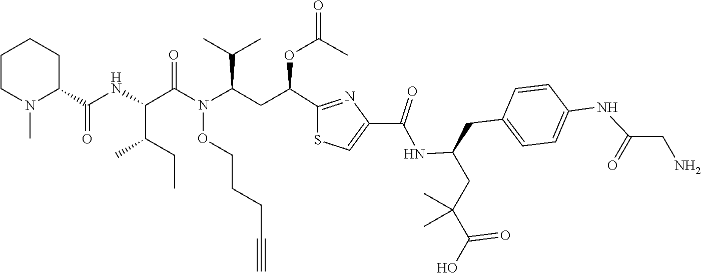 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

or a pharmaceutically acceptable salt thereof, covalently bound to L.

In one embodiment, provided are compounds having the structure of Formula (I)

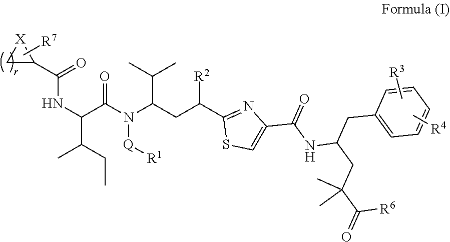

or a pharmaceutically acceptable salt thereof, wherein
X is —O— or —NR⁵,
R⁵ is a hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, or —C(O)—CH₂—NH₂;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, —O—C(O)—NH—(CH₂CH₂O)₃—CH₂NH—C(O)CH₂NH₂, or —O—C(O)—NH—(CH₂CH₂O)₂—(CH₂)₂OH;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
R⁴ is hydrogen or —F
R⁷ when present is —CH₃;
R⁶ is —OH, —NHCH₂C(O)OH, or —NH—C(O)OH;
Q is —CH₂— or —O—;
R¹ is —C₅ alkyl or —C₅ alkynyl;
r is three or four; and
wherein T is not a compound selected from the table below

| ID. | Structure |
|---|---|
| P1 | 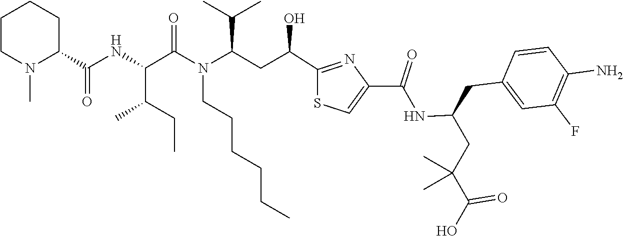 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 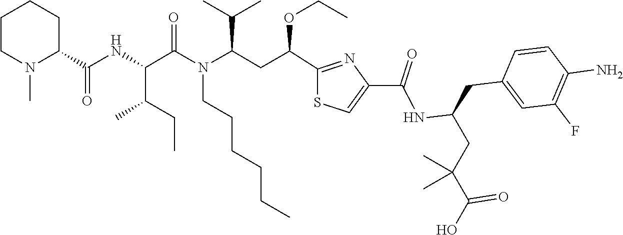 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 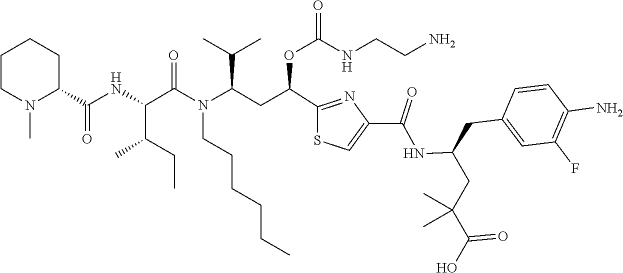 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 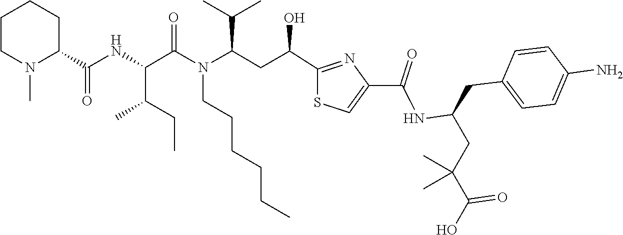 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 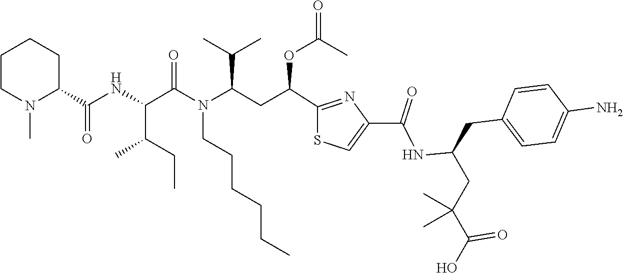 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 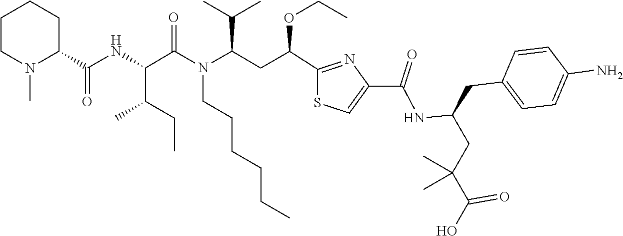 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 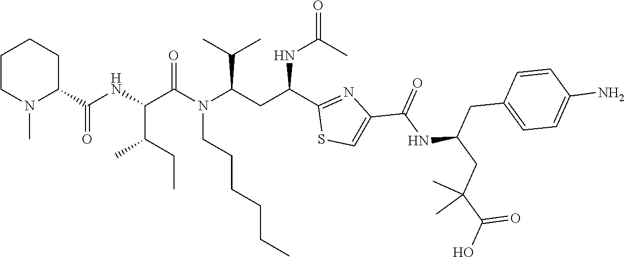 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 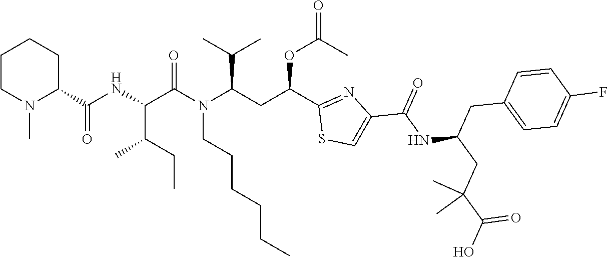 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 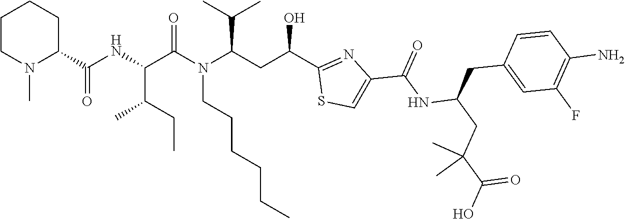 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 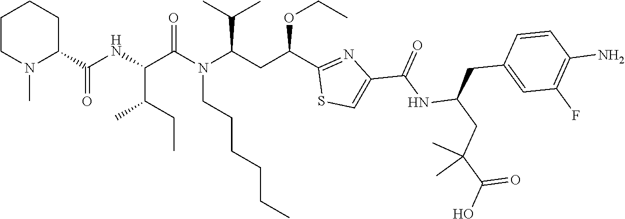 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 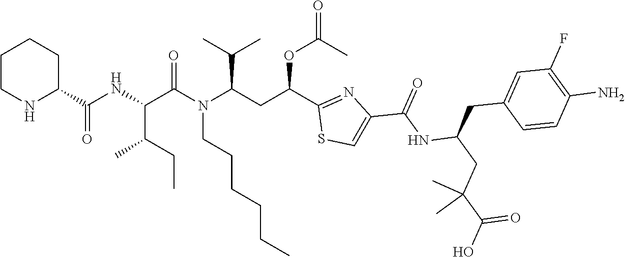 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 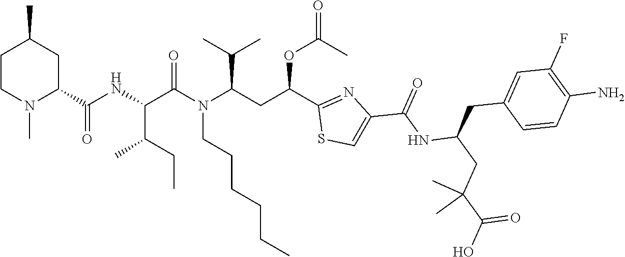 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 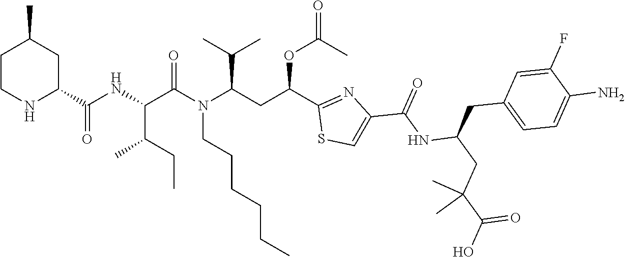 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 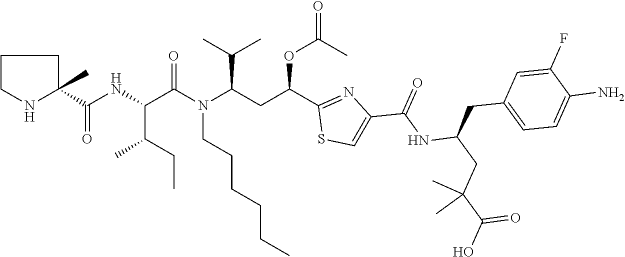 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 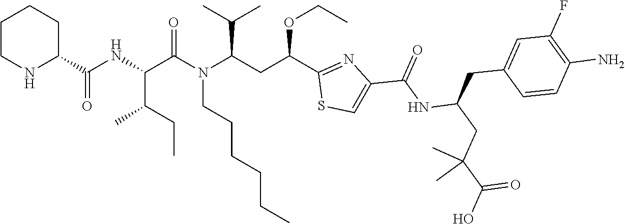 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 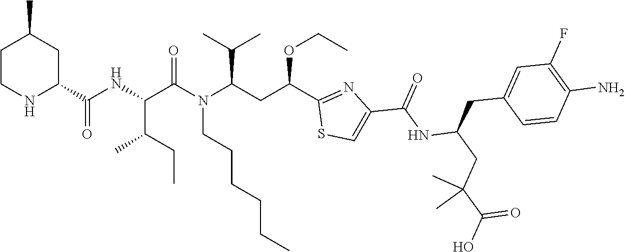 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 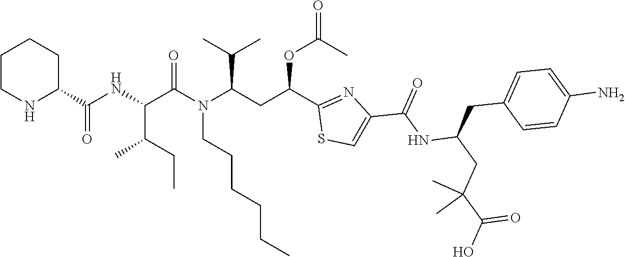 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 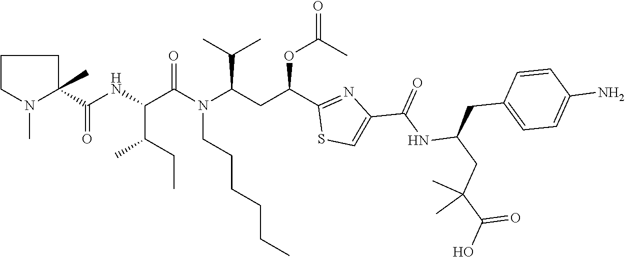 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 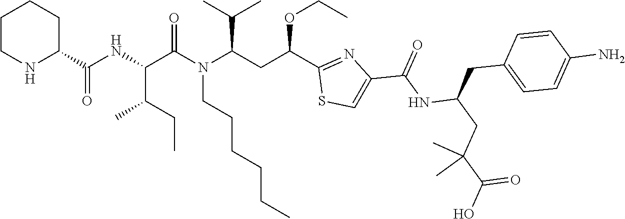 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 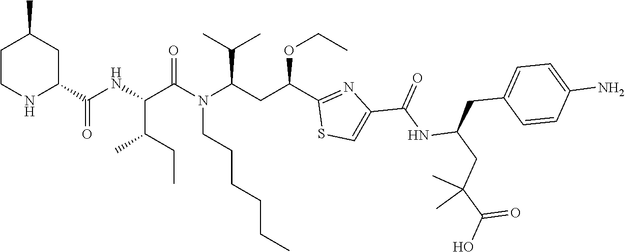 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 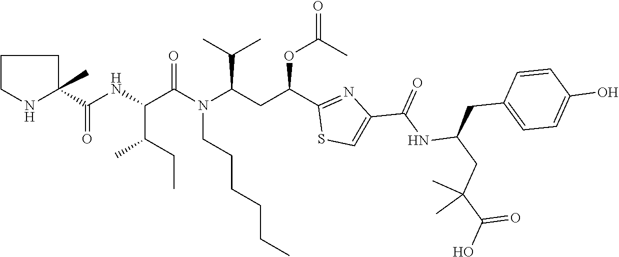 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 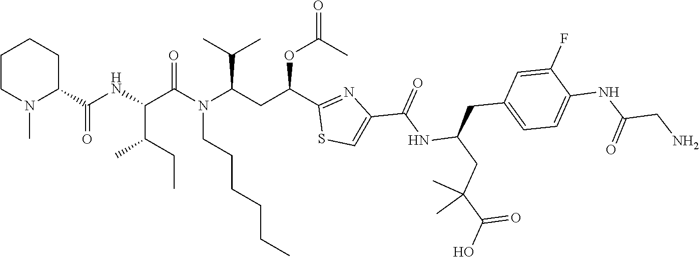 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 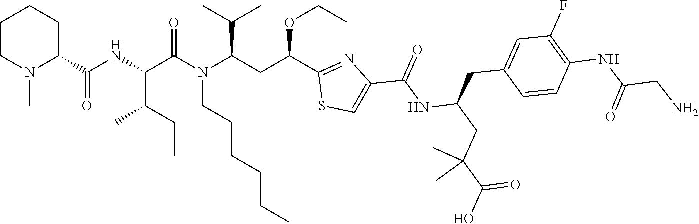 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 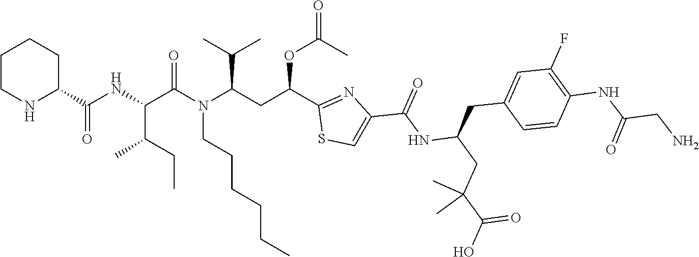 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 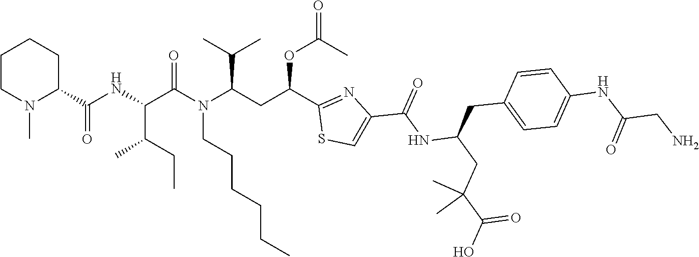 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 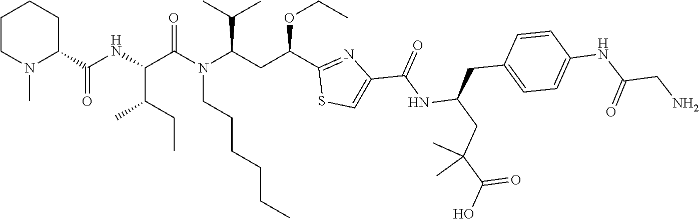 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 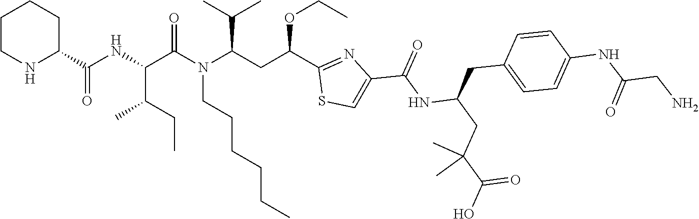 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 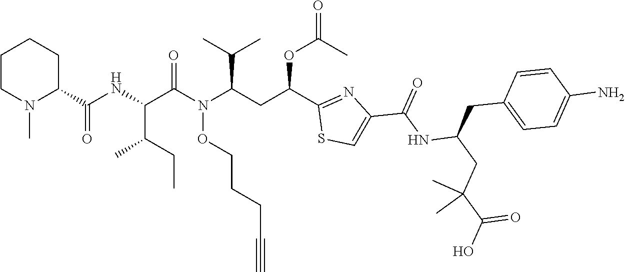 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 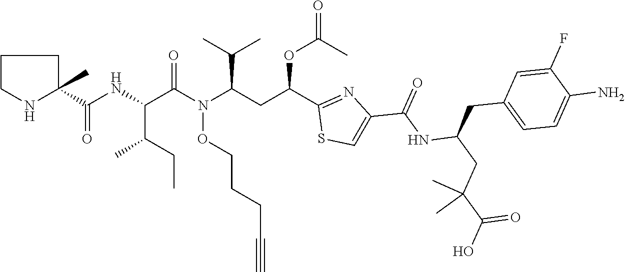 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 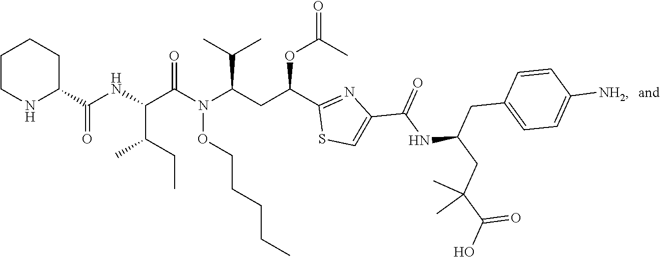 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 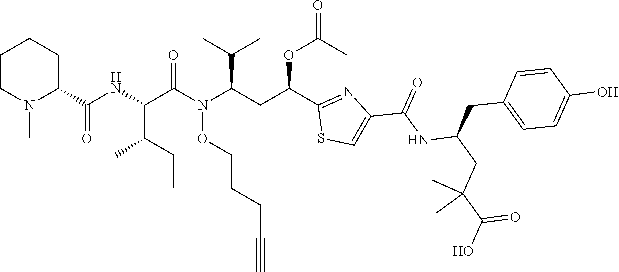 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 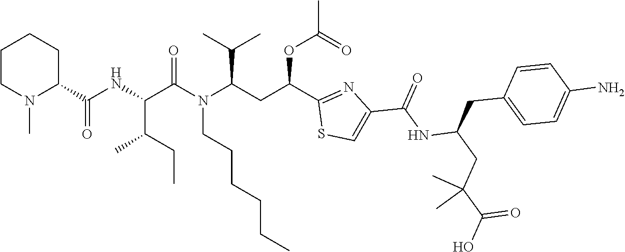 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 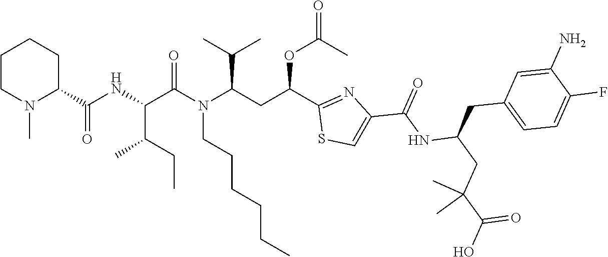 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)c(N)c2)CC(C)(C)C(=O)O)cs1)C(C)C] |
| IVvA | 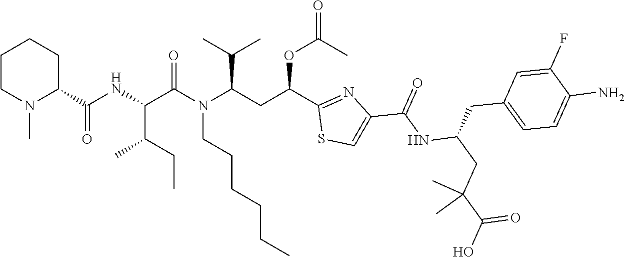 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 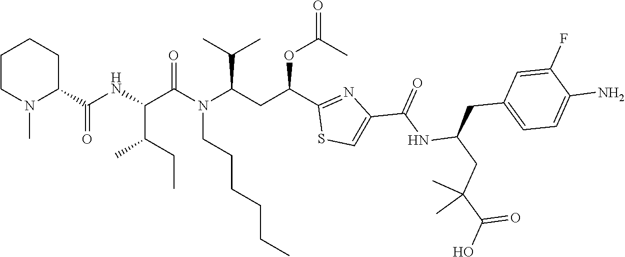 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 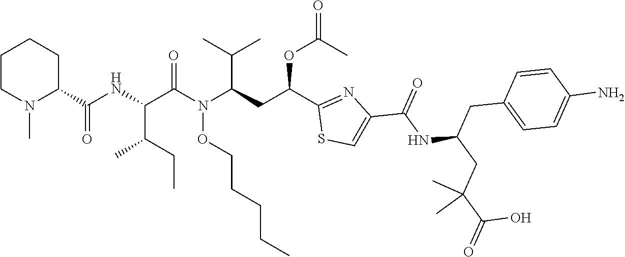 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 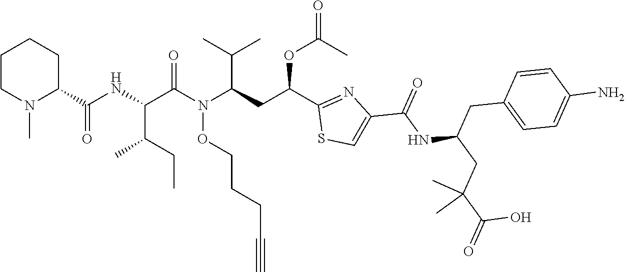 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 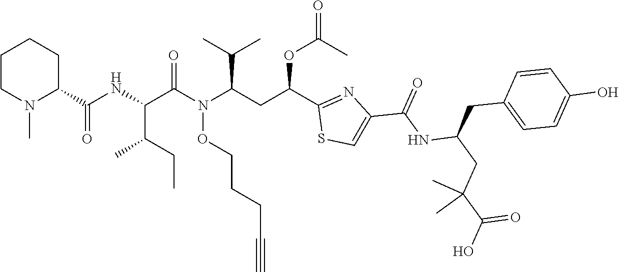 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 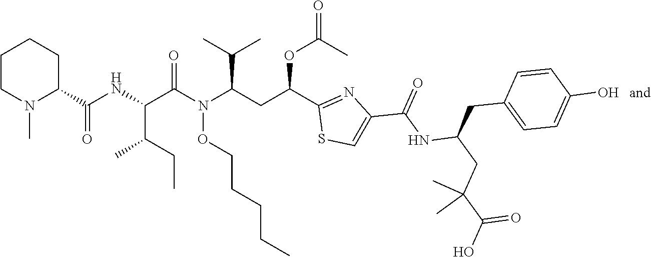 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 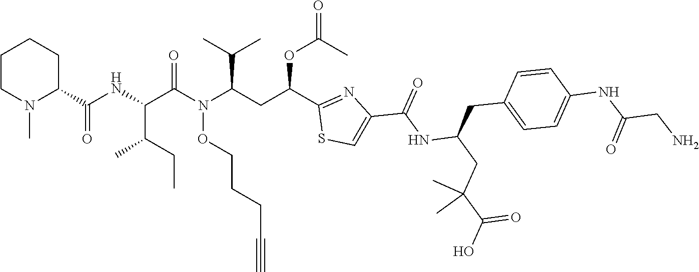 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

In one embodiment, provided are compounds having the structure of Formula (I)

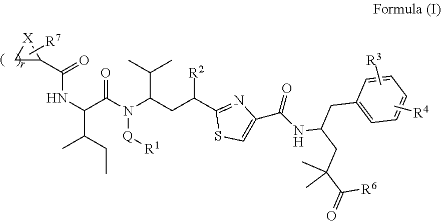

or a pharmaceutically acceptable salt thereof, wherein
X is —O— or —NR⁵,
R⁵ is a hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, or —C(O)—CH₂—NH₂;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, or —O—C(O)—NH₄CH₂CH₂O)₂—(CH₂)₂OH;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, or —NH—CH₂—C(O)—NH—(CH₂CH₂O)₂—(CH₂)₂NH₂;
R⁴ is hydrogen or —F
R⁷ when present is —CH₃;
R⁶ is —OH or —NH—C(O)OH;
Q is —CH₂— or —O—;
R¹ is —C₅ alkyl or —C₅ alkynyl;
r is three or four; and
wherein T not a compound selected from the table below

| ID. | Structure |
|---|---|
| P1 | 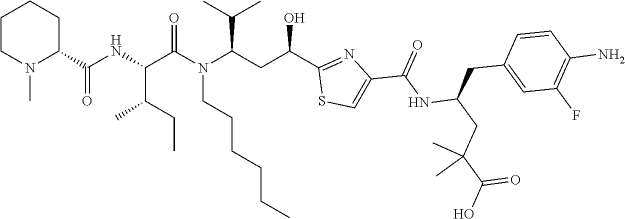 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 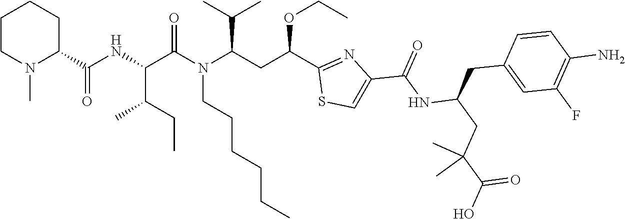 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 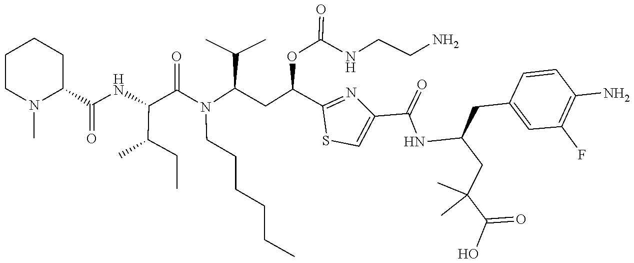 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 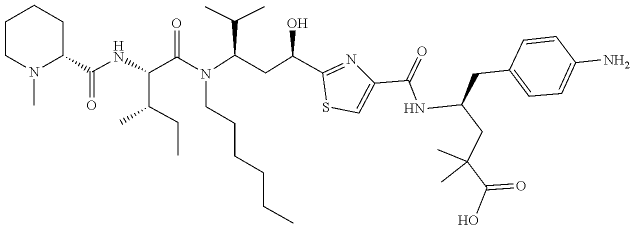 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 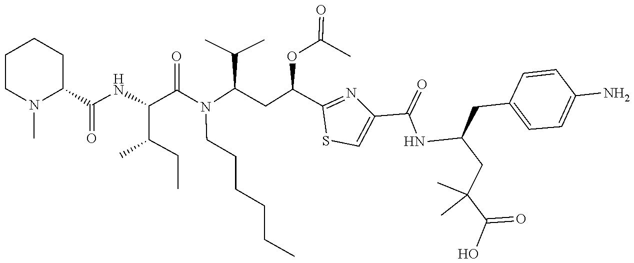 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 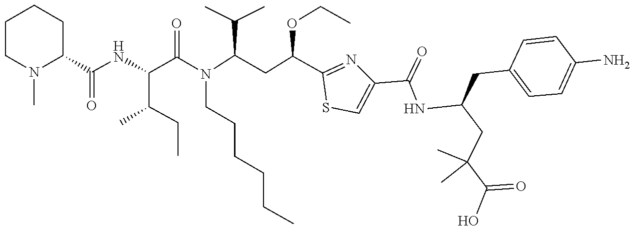 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 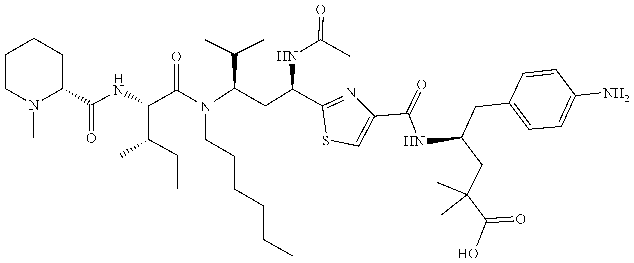 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 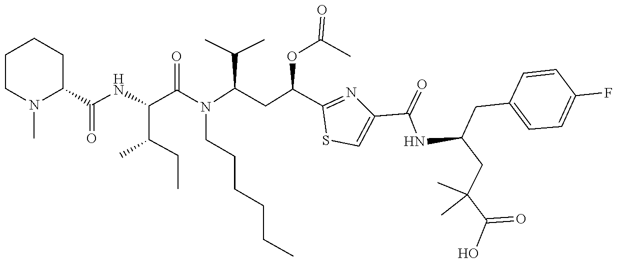 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 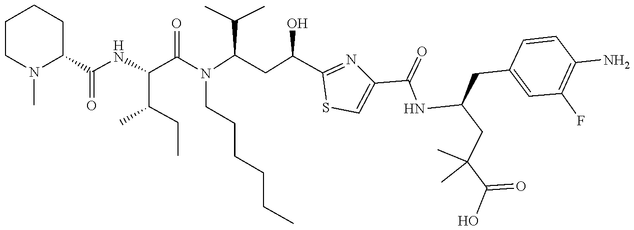 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 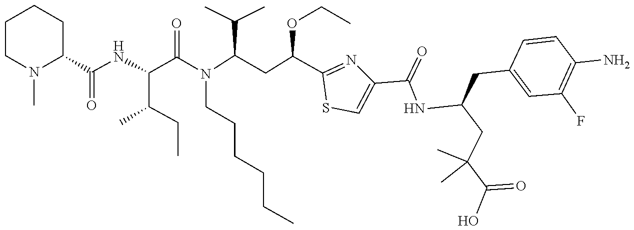 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 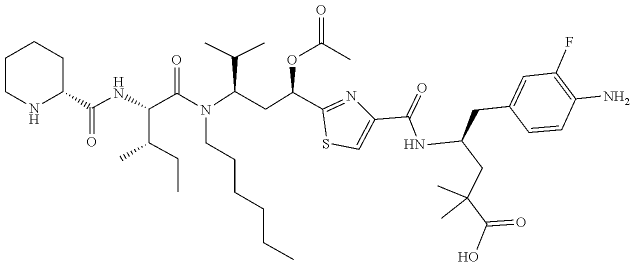 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 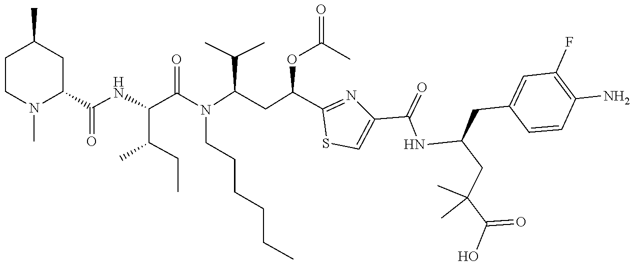 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 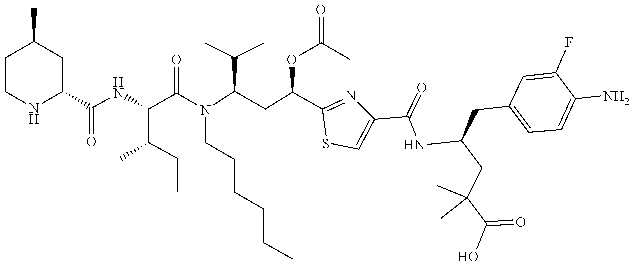 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 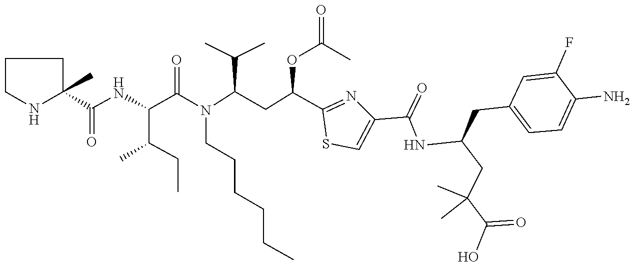 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 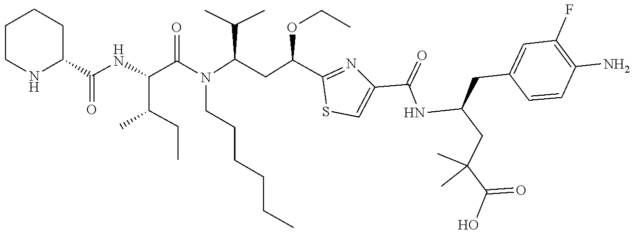 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 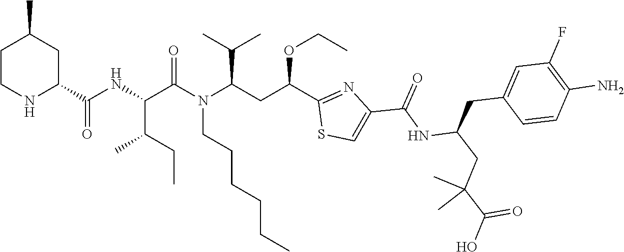 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 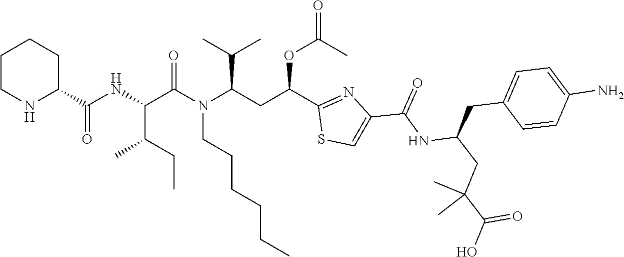 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 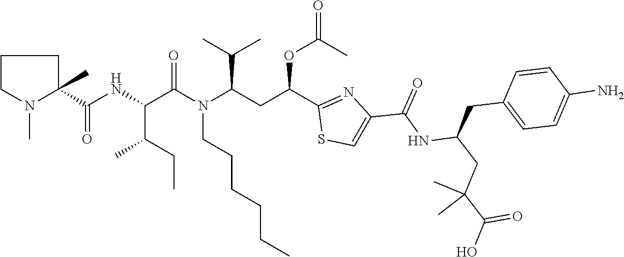 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 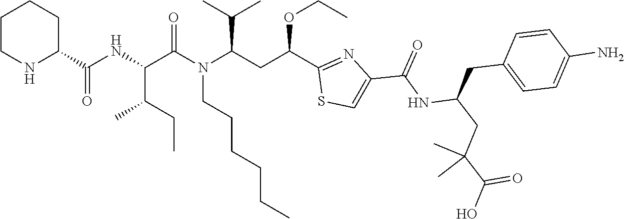 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 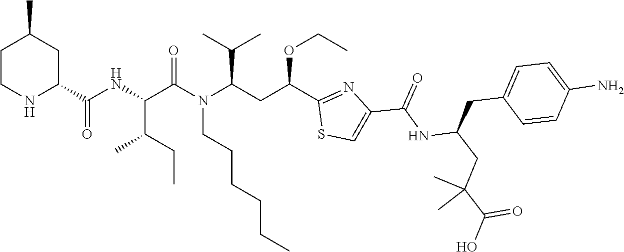 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 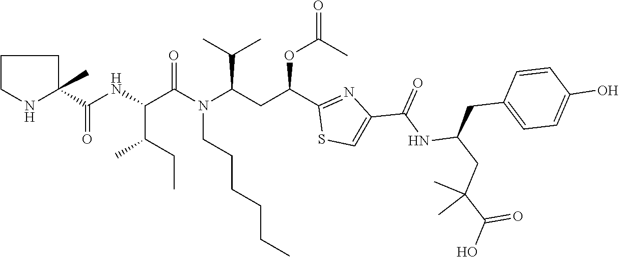 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 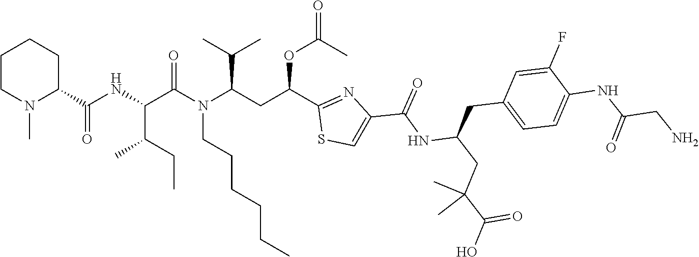 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 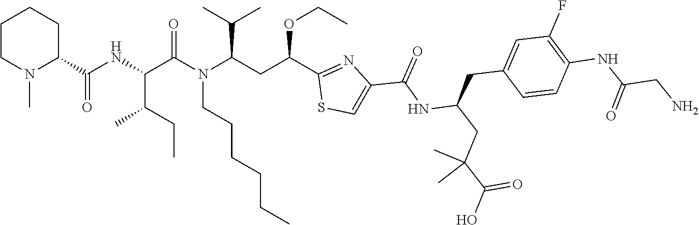 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 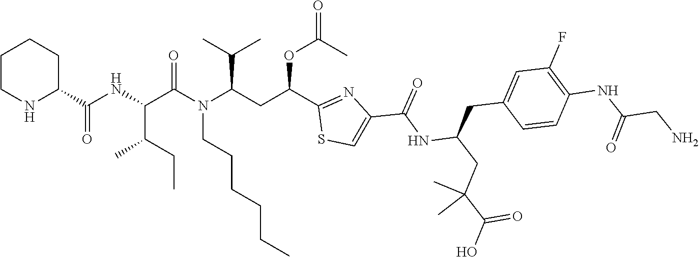 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 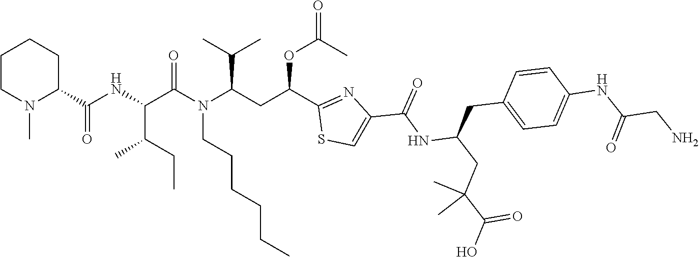 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 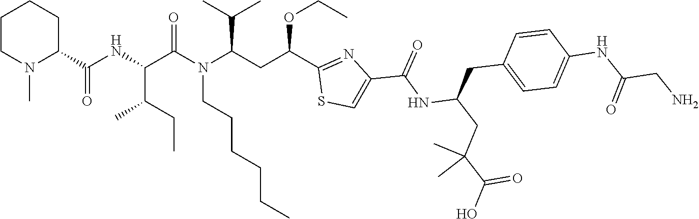 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 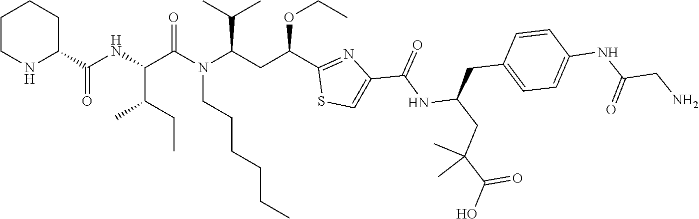 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 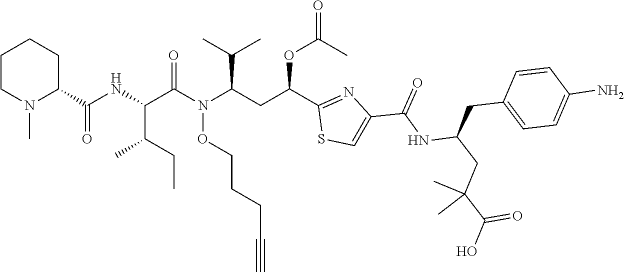 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 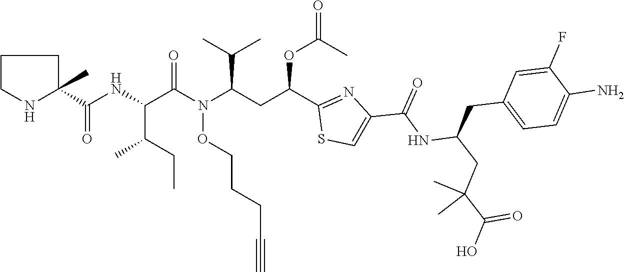 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 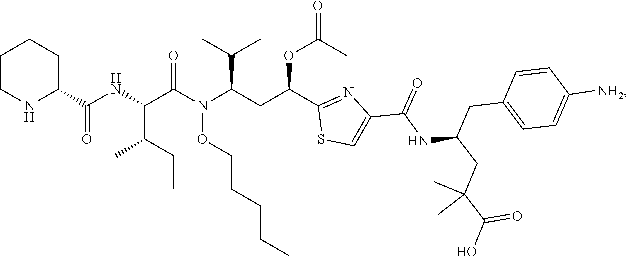 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |

and

| | |
|---|---|
| P51 | 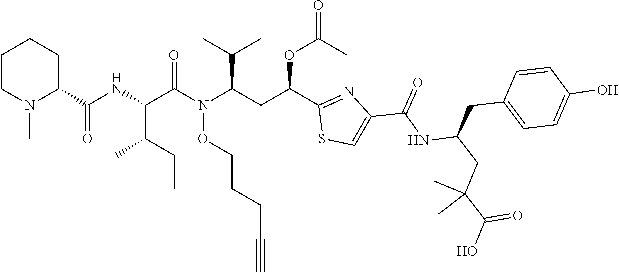 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 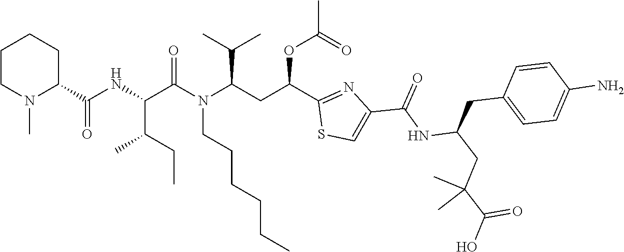 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 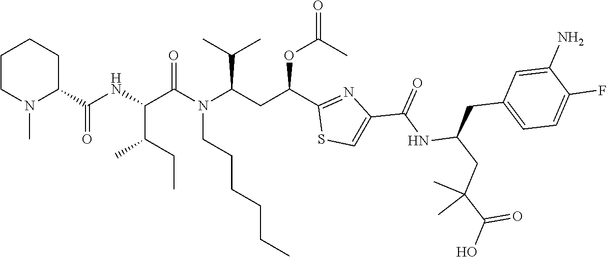 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)c(N)c2)CC(C)(C)C(=O)O)cs1)C(C)C] |
| IVvA | 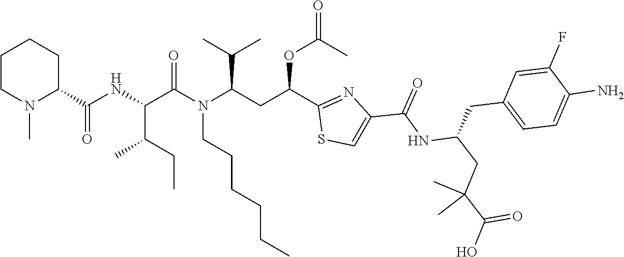 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 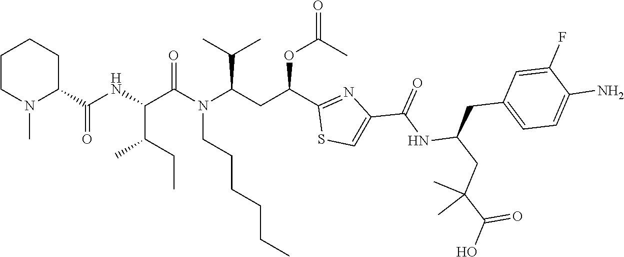 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 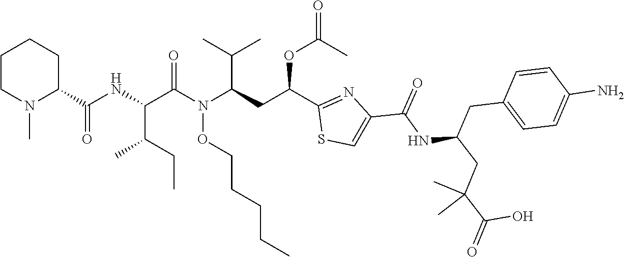 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 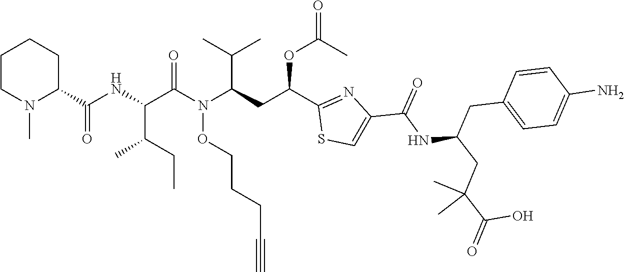 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 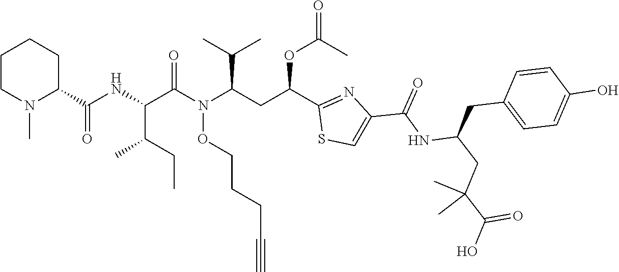 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 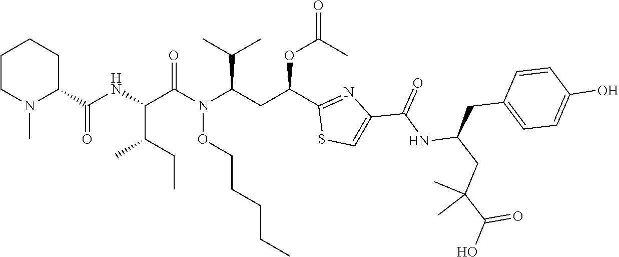 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |

and

| | |
|---|---|
| D-5a | 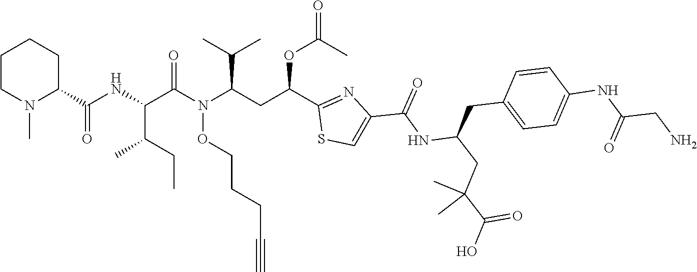 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

or a pharmaceutically acceptable salt thereof, covalently bound to L.

In another embodiment, provided is a linker-payload having the formula

L-T
or a pharmaceutically acceptable salt thereof, wherein
L is a linker covalently bound to T;
T is

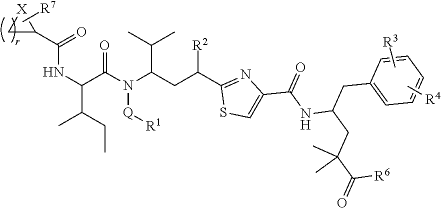

or a residue thereof covalently bound to
L, wherein
X is —O— or —NR⁵;
R⁵ is hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, —C(O)—CH₂—NH₂, a first N-terminal amino acid residue, a first amino acid residue; or
   R⁵ is a covalent bond to L; or
   R⁵ is —(CH₂)₂—OH or —CH₂—C(O)—OH with a covalent bond to L from the terminal oxygen in —(CH₂)₂—OH or —CH₂—C(O)—OH; or
   R⁵ is —(CH₂)₂—NH₂, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue with a covalent bond to L from the nitrogen in any one of —(CH₂)₂—NH₂, —(CH₂)₂—O—(CH₂)₂—NH₂, —(CH₂CH₂—O)₂—(CH₂)₂—NH₂, or —C(O)—CH₂—NH₂, the first N-terminal amino acid residue, or the first amino acid residue;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, —O—C(O)—NH—(CH₂CH₂O)₃—CH₂NH—C(O)CH₂NH₂, or —O—C(O)—NH—(CH₂CH₂O)₂—(CH₂)₂OH; or
   R² is —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH with a covalent bond to L from a terminal oxygen in any one of —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH; or
   R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂O]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂; or
   R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or
   R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂O]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
R⁴ is hydrogen or —F;
R⁷ when present is —CH₃;
R⁶ is —OH, —NHCH₂C(O)OH, or —NH—C(O)OH; or
   R⁶ is —OH, —NHCH₂C(O)OH, or —NH—C(O)OH with a covalent bond to L from the terminal oxygen in any one of —OH, —NHCH₂C(O)OH, or —NH—C(O)OH;
Q is —CH₂— or —O—;
R¹ is —C₅ alkyl, —C₆ alkyl or —C₅ alkynyl;
r is three or four; and
k is an integer from one to thirty;
wherein T is not a compound selected from the table below

| ID. | Structure |
|---|---|
| P1 | 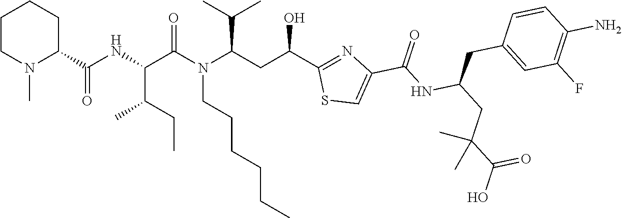 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 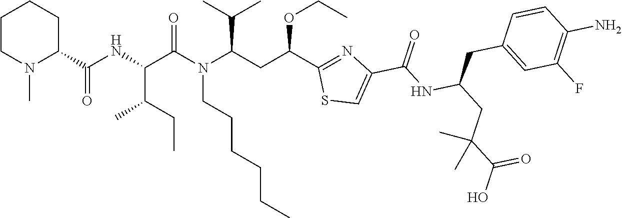 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 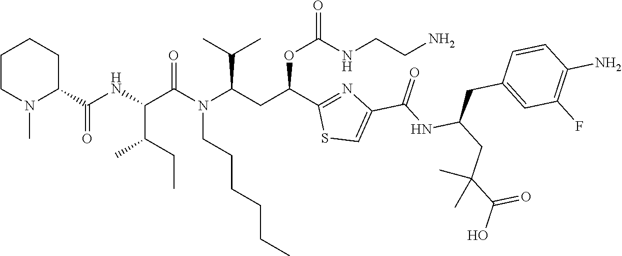 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 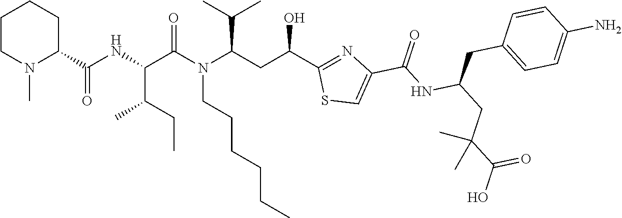 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 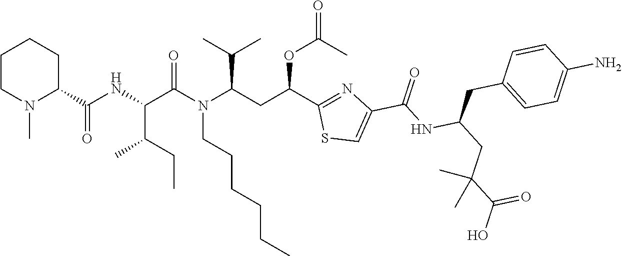 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 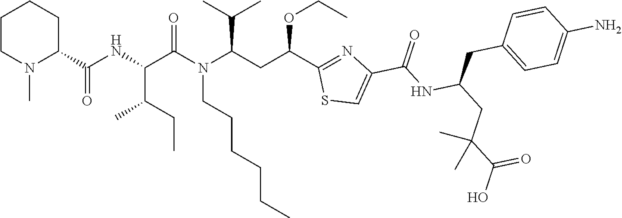 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 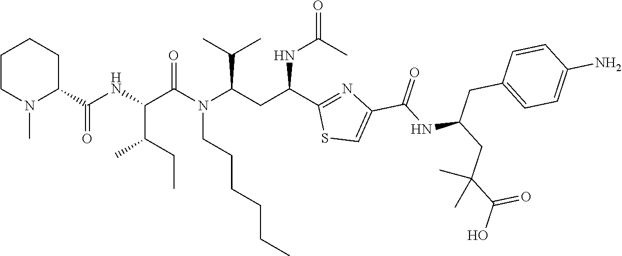 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 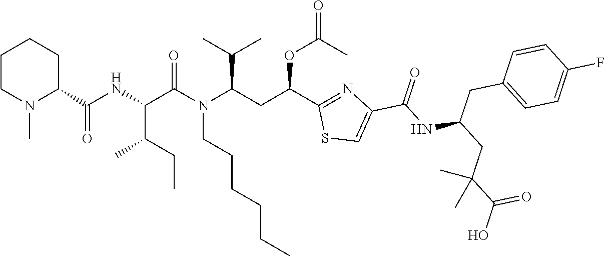 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 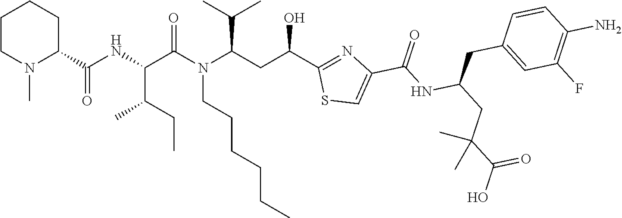 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 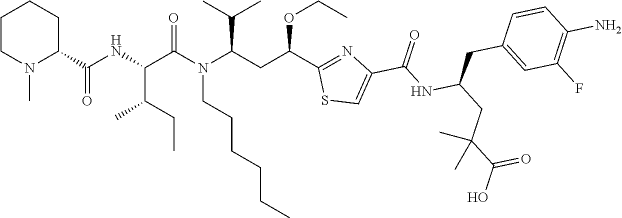 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 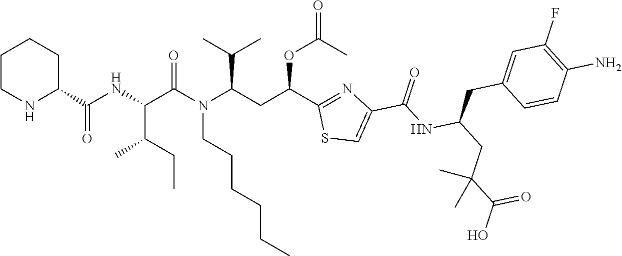 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 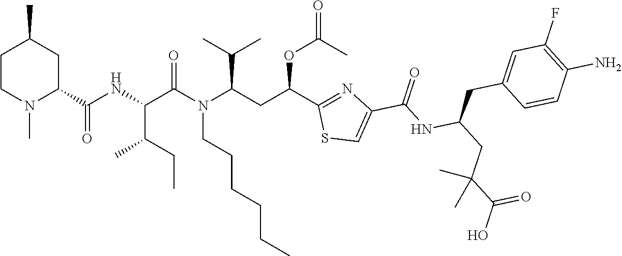 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 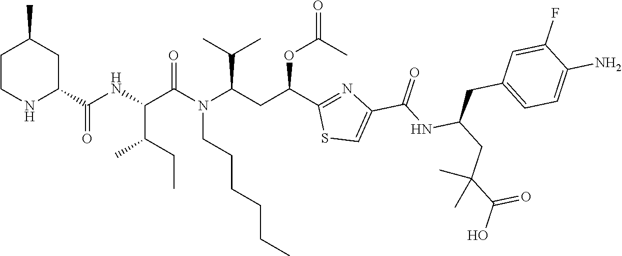 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 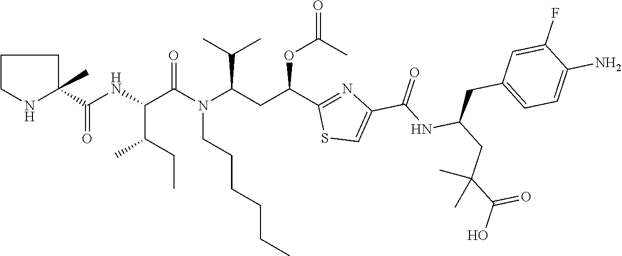 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 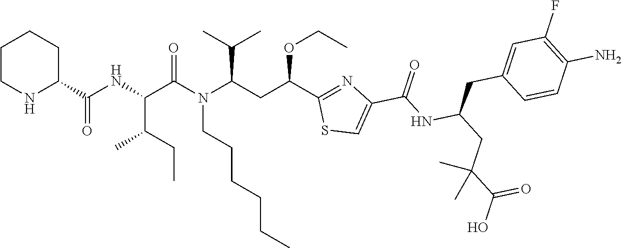 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 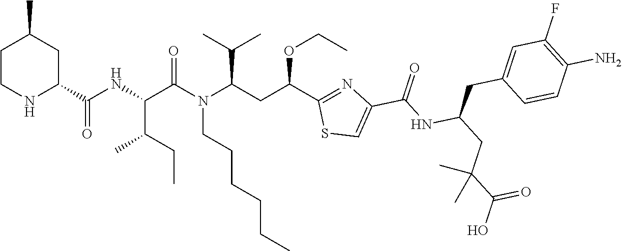 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 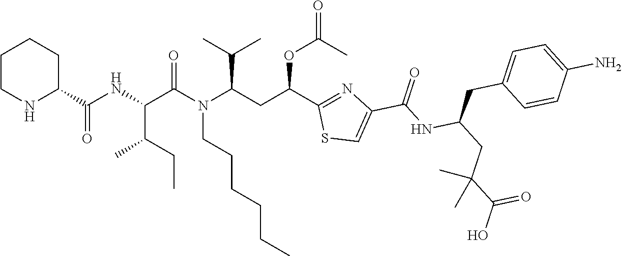 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 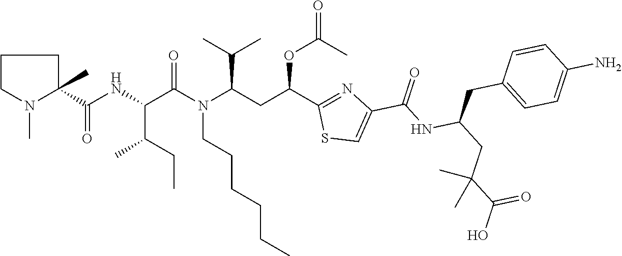 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 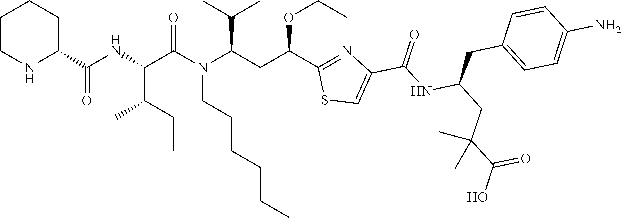 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 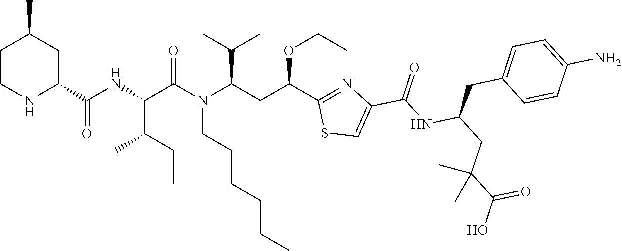 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 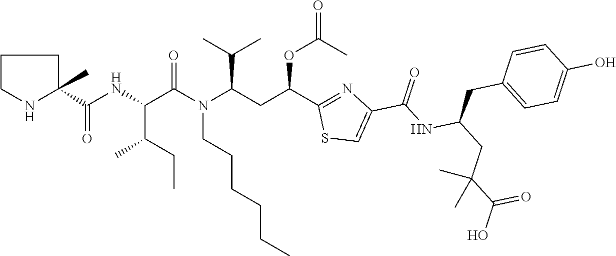 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 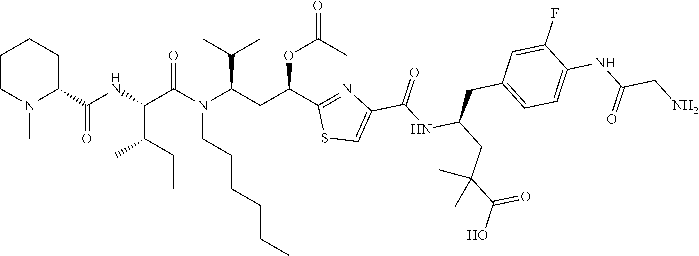 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 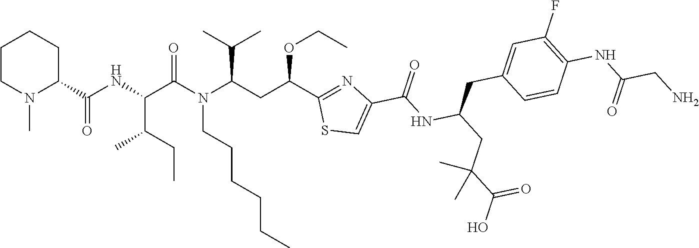 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 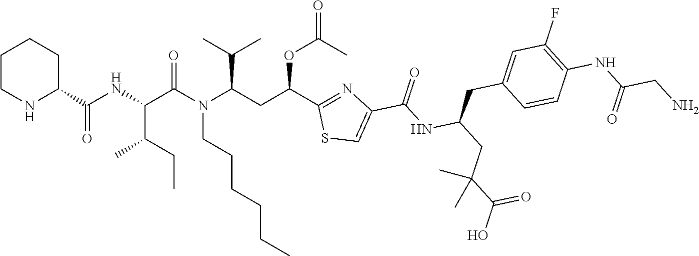 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 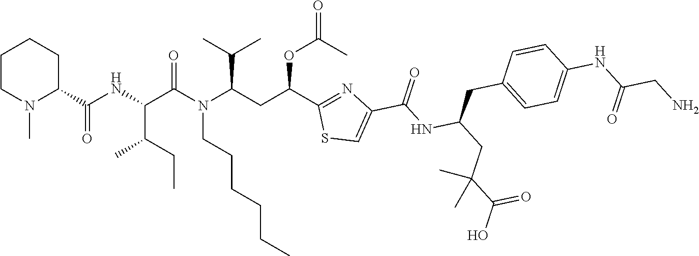 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 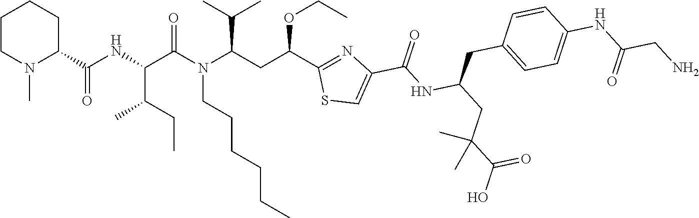 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 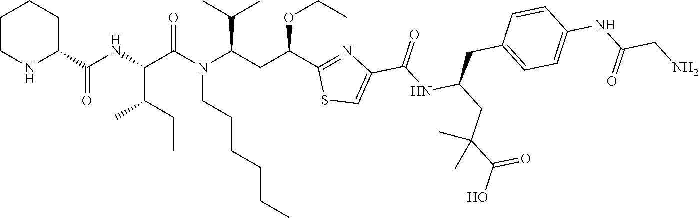 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 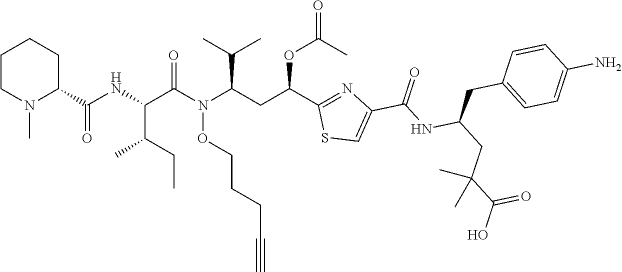 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 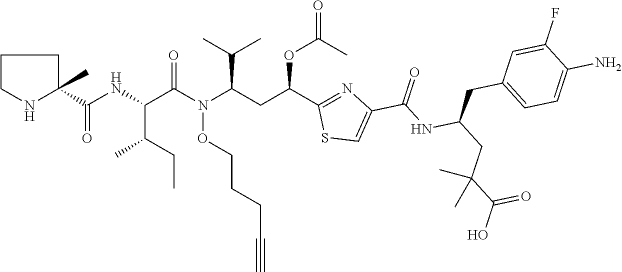 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 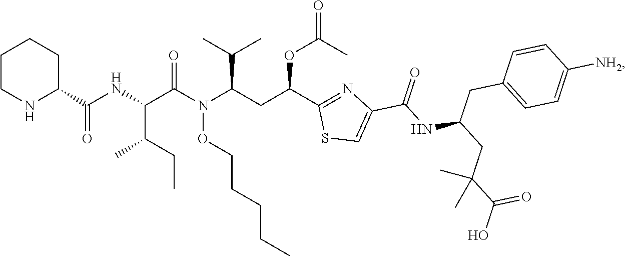 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 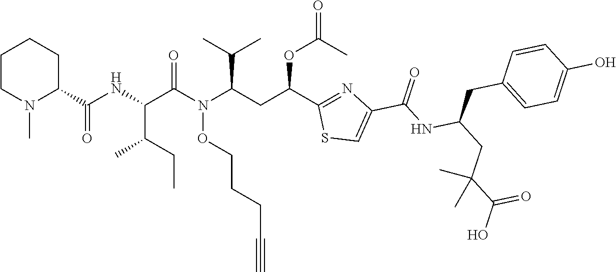 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 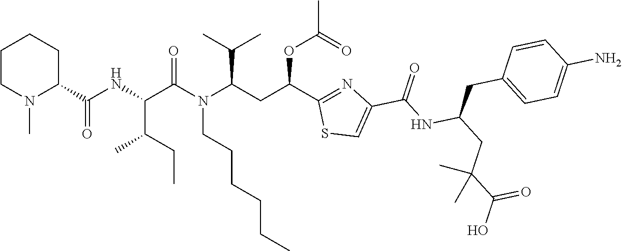 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 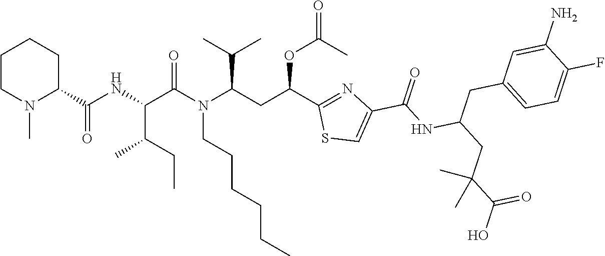 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 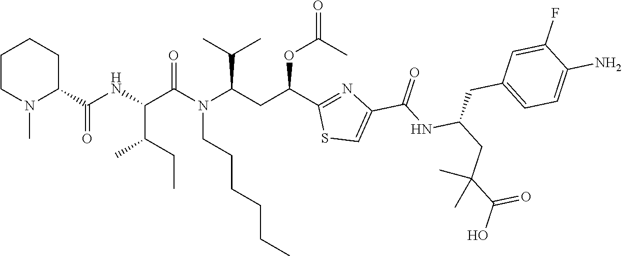 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 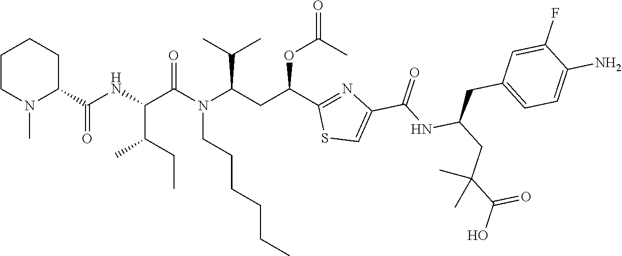 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 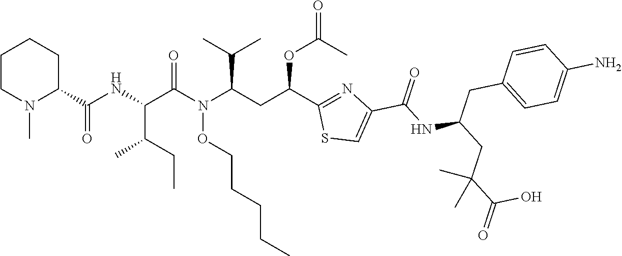 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 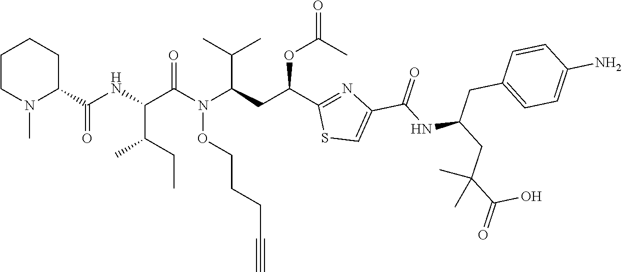 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 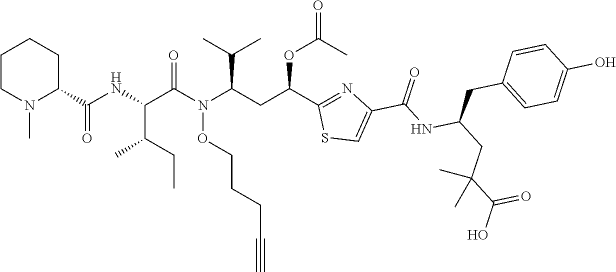 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 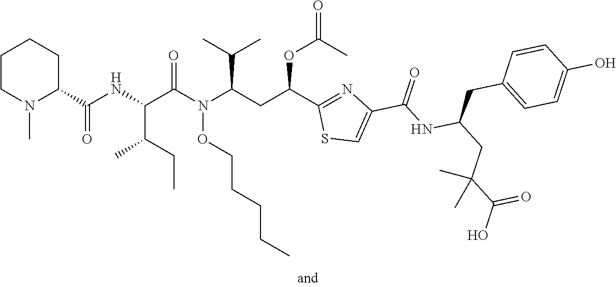 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 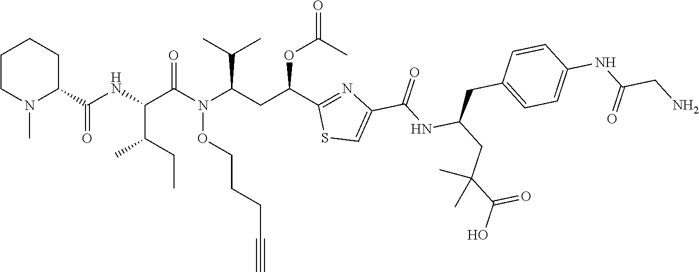 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

In another embodiment, provided is a linker-payload having the formula

L-T
or a pharmaceutically acceptable salt thereof, wherein
L is a linker covalently bound to T;
T is

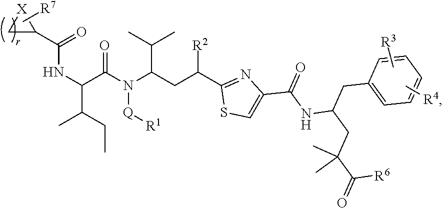

wherein
X is —O— or —NR⁵,
R⁵ is hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue;
R² is —O—C(O)—NH—CH₂—CH(OH)—CH₂OH, —N—C(O)CH₃, —O—CH₂CH₃, —O—(CH₂)₃—OH, —O—C(O)CH₃, —O—C(O)—NH—(CH₂)₂—OH, or —O—C(O)—NH—(CH₂CH₂O)₂—(CH₂)₂OH;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, or —NH—CH₂—C(O)—NH—(CH₂CH₂O)₂—(CH₂)₂NH₂;
R⁴ is hydrogen or —F
R⁷ when present is —CH₃;
R⁶ is —OH or —NH—C(O)OH;
Q is —CH₂— or —O—;
R⁴ is —C₅ alkyl, —C₆ alkyl or —C₅ alkynyl;
r is three or four; and
wherein T is not a compound selected from the table below

| ID. | Structure |
|---|---|
| P1 | 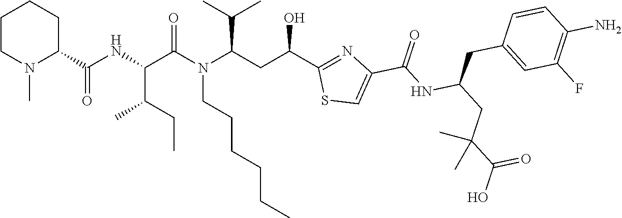 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 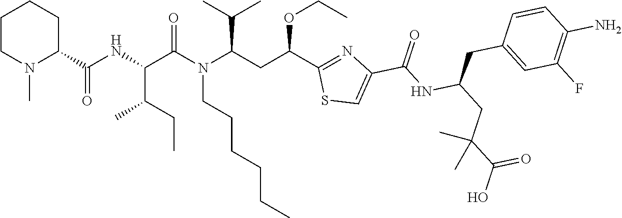 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 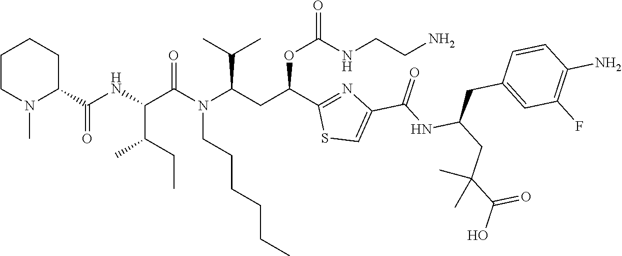 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 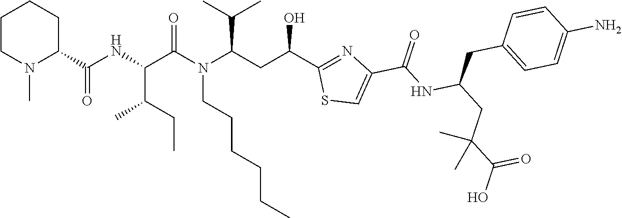 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 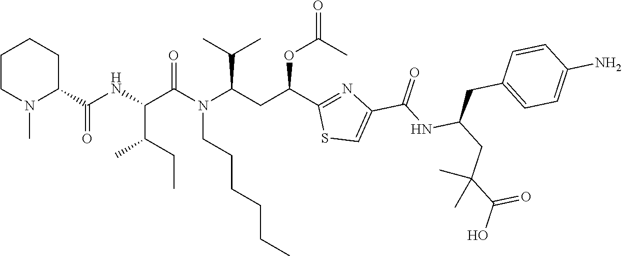 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 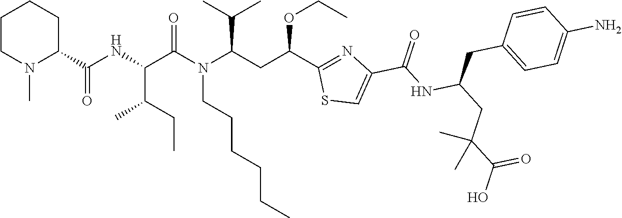 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 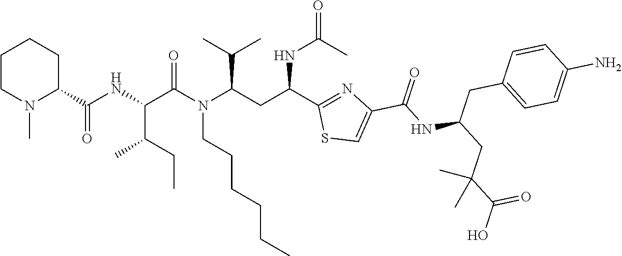 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 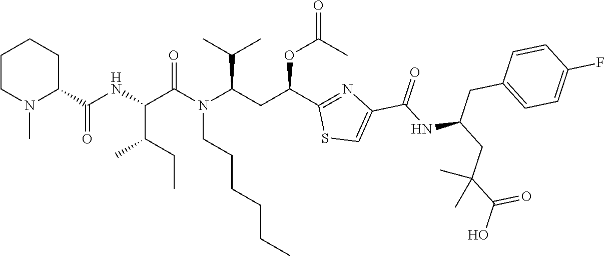 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 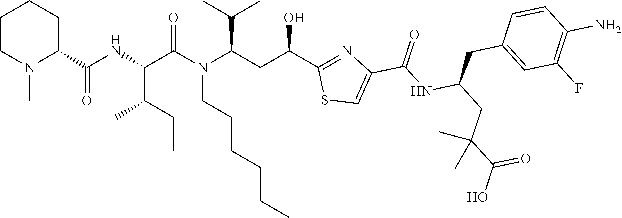 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 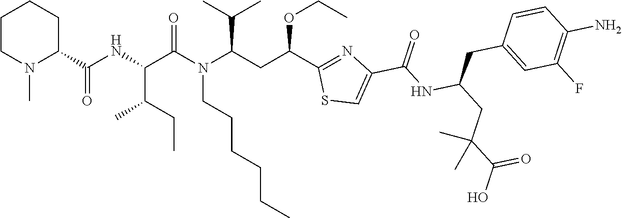 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 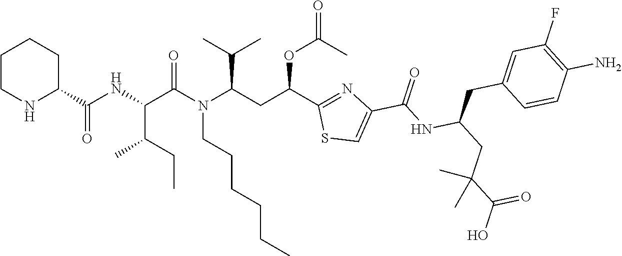 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 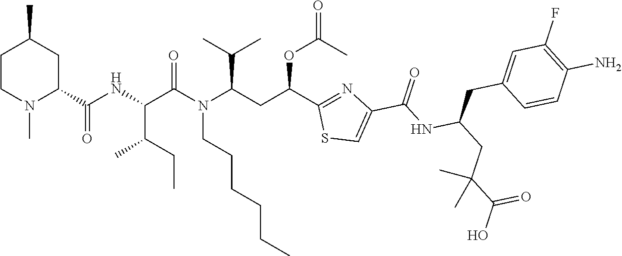 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 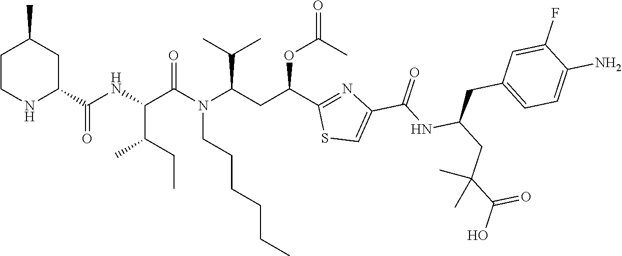 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 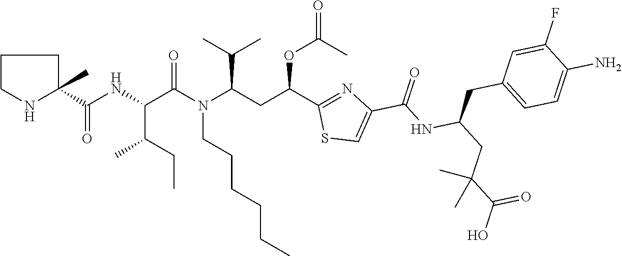 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 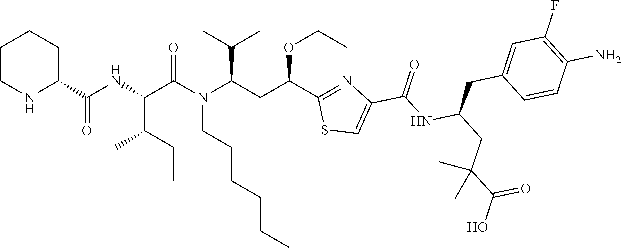 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 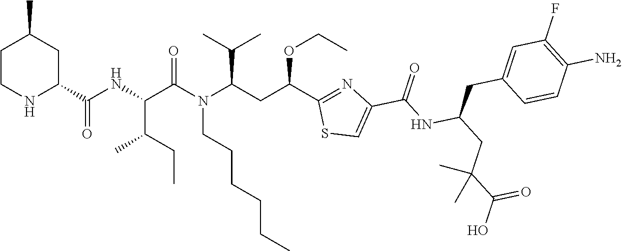 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 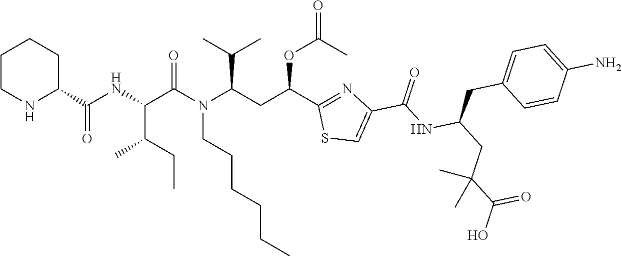 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 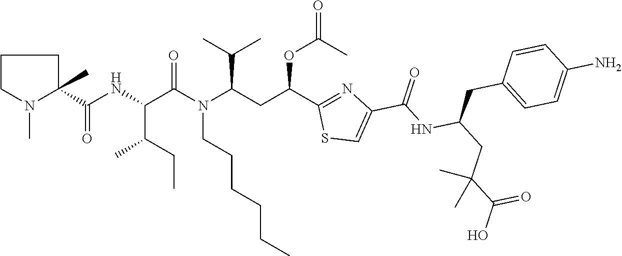 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 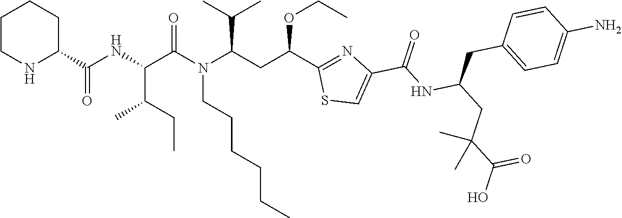 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 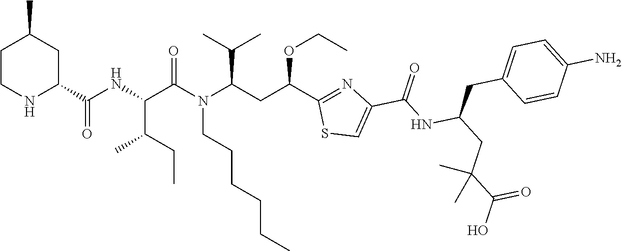 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 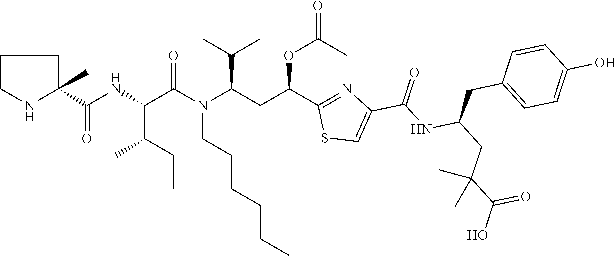 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 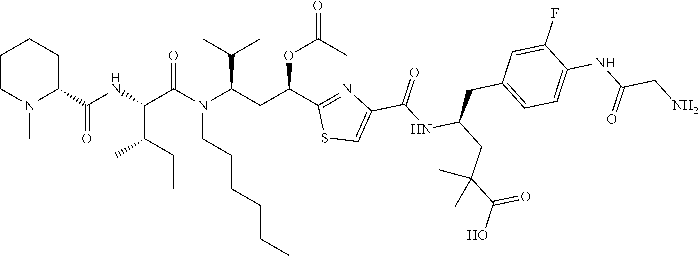 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 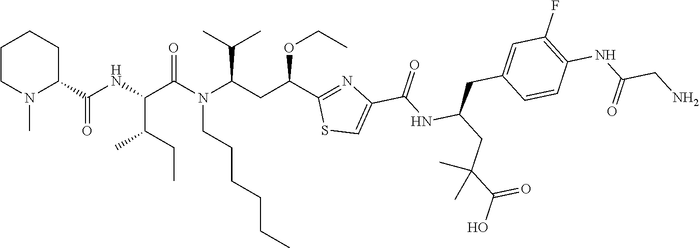 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 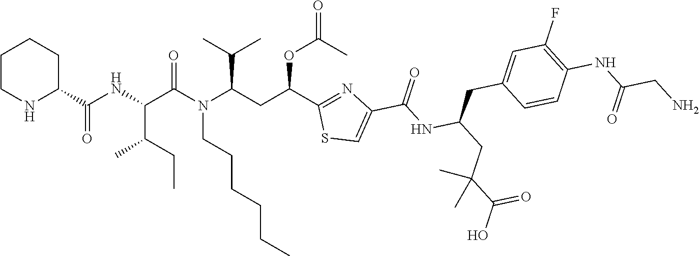 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 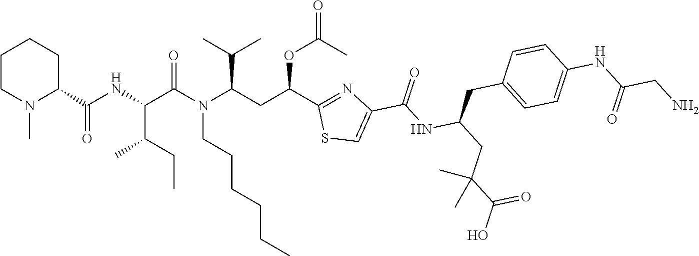 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 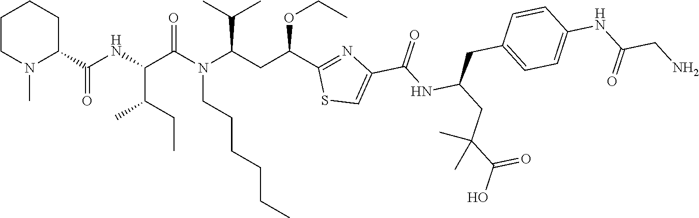 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 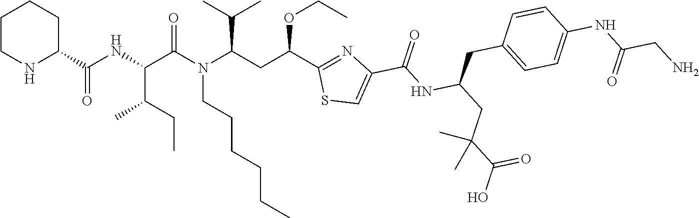 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 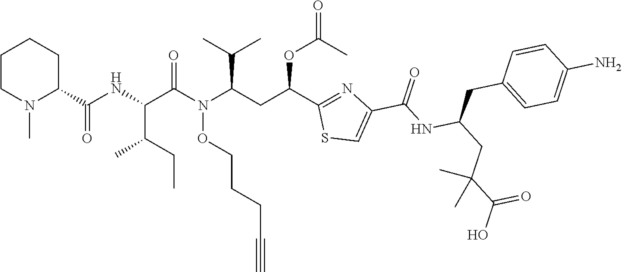 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 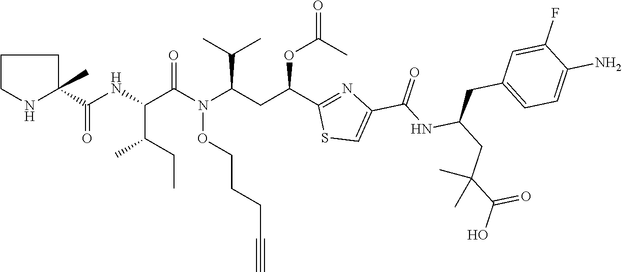 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 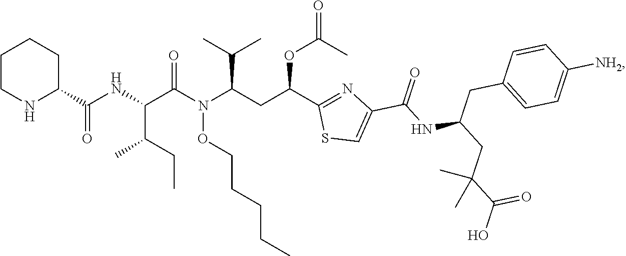 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 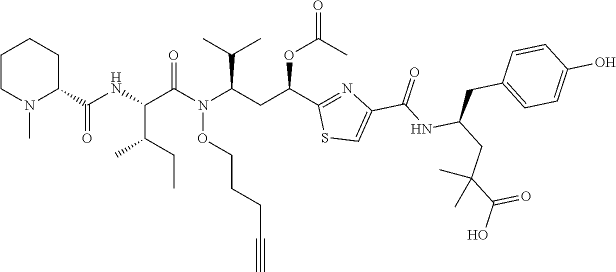 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 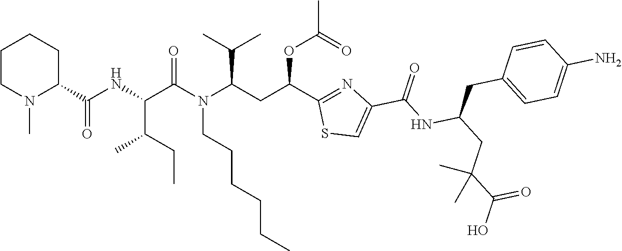 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 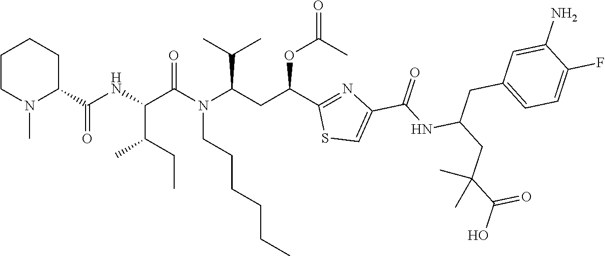 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 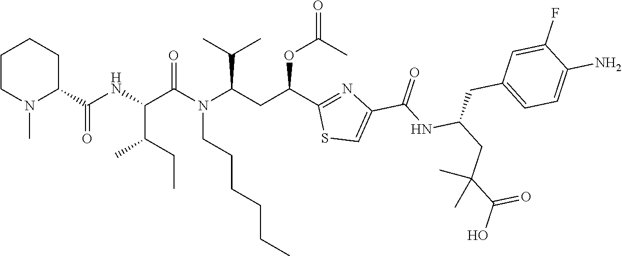 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 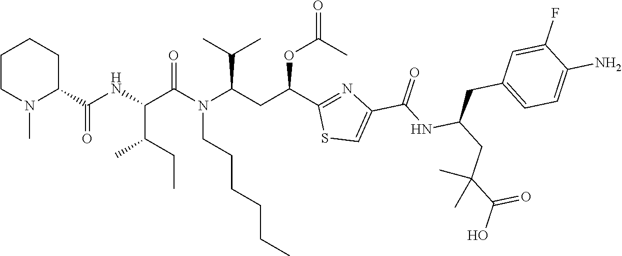 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 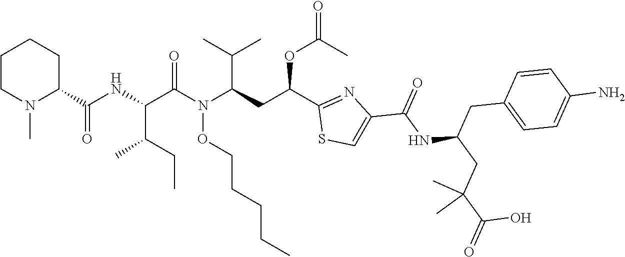 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 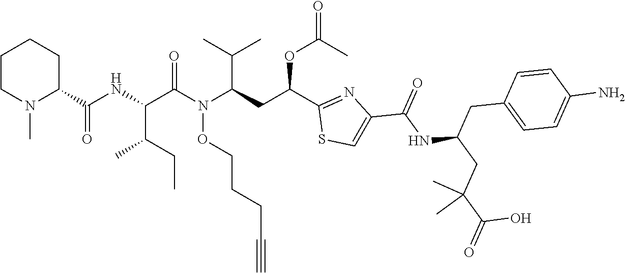 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 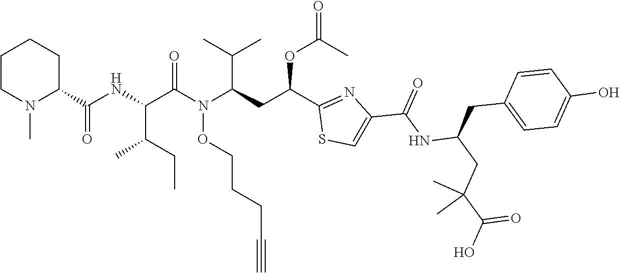 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 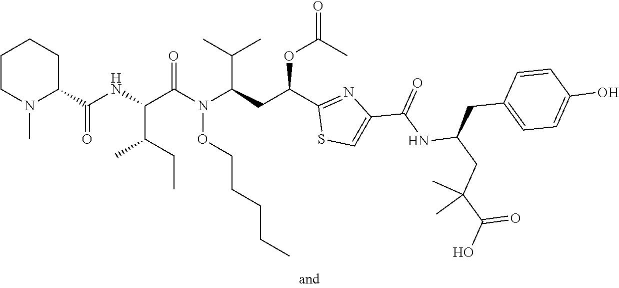 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 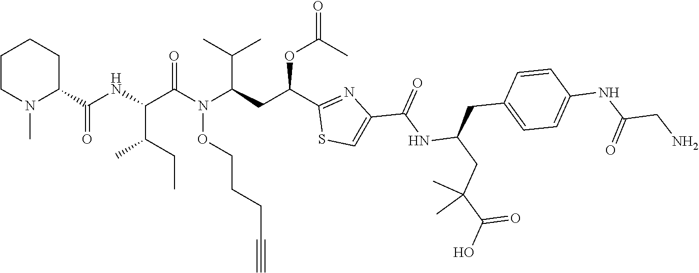 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

or a pharmaceutically acceptable salt thereof.

In another embodiment, set forth herein is an antibody-drug conjugate including an antibody or antigen-binding fragment thereof, wherein said antibody or antigen-binding fragment thereof is conjugated to a compound as described herein.

In another embodiment, set forth herein are methods for making the compounds, linker-payloads, antibody-drug conjugates, and compositions described herein.

### BRIEF DESCRIPTIONS OF THE DRAWING

FIGS. 1-19 show synthetic chemistry schemes for tubulysin payloads, and tubulysin linker-payloads, wherein each are capable of conjugation to or conjugated to an antibody or antigen-binding fragment thereof.

### DESCRIPTION OF EXEMPLARY EMBODIMENTS

Provided herein are compounds, compositions, and methods useful for treating for example, cancer in a subject.

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term provided herein, the definitions below prevail unless stated otherwise.

As used herein, “alkyl” refers to a monovalent and saturated hydrocarbon radical moiety. Alkyl is optionally substituted and can be linear, branched, or cyclic (i.e., cycloalkyl). Alkyl includes, but is not limited to, those radicals having 1-20 carbon atoms (i.e., C₁₋₂₀ alkyl); 1-12 carbon atoms (i.e., C₁₋₁₂ alkyl); 1-10 carbon atoms (i.e., C₁₋₁₀ alkyl); 1-8 carbon atoms (i.e., C₁₋₈ alkyl); 5-10 carbon atoms (i.e., C₅₋₁₀ alkyl); 1-5 carbon atoms (i.e., C₁₋₅ alkyl); 1-6 carbon atoms (i.e., C₁₋₆ alkyl); and 1-3 carbon atoms (i.e., C₁₋₃ alkyl). Examples of alkyl moieties include, but are not limited to, methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl. A pentyl moiety includes, but is not limited to, n-pentyl and i-pentyl. A hexyl moiety includes, but is not limited to, n-hexyl.

As used herein, “alkylene” refers to a divalent alkyl group. Unless specified otherwise, alkylene includes, but is not limited to, 1-20 carbon atoms. The alkylene group is optionally substituted as described herein for alkyl or elsewhere. In some embodiments, alkylene is unsubstituted.

Designation of an amino acid or amino acid residue without specifying its stereochemistry is intended to encompass the L-form of the amino acid, the D-form of the amino acid, or a racemic mixture thereof.

As used herein, “haloalkyl” refers to alkyl, as defined above, wherein the alkyl includes at least one substituent selected from a halogen, for example, fluorine (F), chlorine (C₁), bromine (Br), or iodine (I). Examples of haloalkyl include, but are not limited to, —CF₃, —CH₂CF₃, —CCl₂F, and —CCl₃.

As used herein, “alkenyl” refers to a monovalent hydrocarbon radical moiety containing at least two carbon atoms and one or more non-aromatic carbon-carbon double bonds. Alkenyl is optionally substituted and can be linear, branched, or cyclic. Alkenyl includes, but is not limited to, those radicals having 2-20 carbon atoms (i.e., C₂₋₂₀ alkenyl); 2-12 carbon atoms (i.e., C₂₋₁₂ alkenyl); 2-8 carbon atoms (i.e., C₂₋₈ alkenyl); 2-6 carbon atoms (i.e., C₂₋₆ alkenyl); and 2-4 carbon atoms (i.e., C₂₋₄ alkenyl). Examples of alkenyl moieties include, but are not limited to, vinyl, propenyl, butenyl, and cyclohexenyl.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms (i.e., C₂₋₂₀ alkynyl); 2-12 carbon atoms (i.e., C₂₋₁₂ alkynyl); 2-8 carbon atoms (i.e., C₂₋₈ alkynyl); 2-6 carbon atoms (i.e., C₂₋₆ alkynyl); and 2-4 carbon atoms (i.e., C₂₋₄ alkynyl). Examples of alkynyl moieties include, but are not limited to ethynyl, propynyl, and butynyl.

As used herein, “alkoxy” refers to a monovalent and saturated hydrocarbon radical moiety wherein the hydrocarbon includes a single bond to an oxygen atom and wherein the radical is localized on the oxygen atom (e.g., CH₃CH₂—O· for ethoxy). Alkoxy substituents bond to the compound which they substitute through the oxygen atom of the alkoxy substituent. Alkoxy is optionally substituted and can be linear, branched, or cyclic (i.e., cycloalkoxy). Alkoxy includes, but is not limited to, those having 1-20 carbon atoms (i.e., C₁₋₂₀ alkoxy); 1-12 carbon atoms (i.e., C₁₋₁₂ alkoxy); 1-8 carbon atoms (i.e., C₁₋₈ alkoxy); 1-6 carbon atoms (i.e., C₁₋₆ alkoxy); and 1-3 carbon atoms (i.e., C₁₋₃ alkoxy). Examples of alkoxy moieties include, but are not limited to, methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy.

As used herein, “haloalkoxy” refers to alkoxy, as defined above, wherein the alkoxy includes at least one substituent selected from a halogen (e.g., F, Cl, Br, or I).

As used herein, “aryl” refers to a monovalent moiety that is a radical of an aromatic compound wherein the ring atoms are carbon atoms. Aryl is optionally substituted and can be monocyclic or polycyclic (e.g., bicyclic or tricyclic). Examples of aryl moieties include, but are not limited to, those having 6 to 20 ring carbon atoms (i.e., C₆₋₂₀ aryl); 6 to 15 ring carbon atoms (i.e., C₆₋₁₅ aryl), and 6 to 10 ring carbon atoms (i.e., C₆₋₁₀ aryl). Examples of aryl moieties include, but are limited to, phenyl, naphthyl, azulenyl, anthryl, phenanthryl, and pyrenyl.

As used herein, “arylalkyl” refers to a monovalent moiety that is a radical of an alkyl compound, wherein the alkyl compound is substituted with an aromatic substituent (i.e., the aromatic moiety includes a single bond to an alkyl group and wherein the radical is localized on the alkyl group). An arylalkyl group bonds to the illustrated chemical structure via the alkyl group. An arylalkyl can be represented by the structure(s)

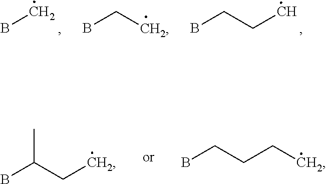

wherein B is an aromatic moiety (e.g., aryl or phenyl). Arylalkyl is optionally substituted (i.e., the aryl group and/or the alkyl group, can be substituted as disclosed herein). Examples of arylalkyl include, but are not limited to, benzyl.

As used herein, “alkylaryl” refers to a monovalent moiety that is a radical of an aryl compound, wherein the aryl compound is substituted with an alkyl substituent (i.e., the aryl moiety includes a single bond to an alkyl group and wherein the radical is localized on the aryl group). An alkylaryl group bonds to the illustrated chemical structure via the aryl group. An alkylaryl can be represented by the structure(s)

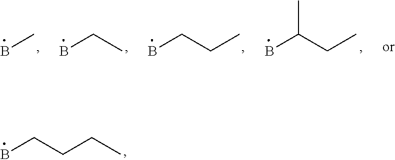

wherein B is an aromatic moiety (e.g., phenyl). Alkylaryl is optionally substituted (i.e., the aryl group and/or the alkyl group can be substituted as disclosed herein). Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical (i.e., the aromatic compound includes a single bond to an oxygen atom and wherein the radical is localized on the oxygen atom, e.g.,

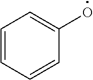

for phenoxy). Aryloxy substituents bond to the compound in which they substitute through the oxygen atom. Aryloxy is optionally substituted. Aryloxy includes, but is not limited to, those radicals having 6 to 20 ring carbon atoms (i.e., C₆₋₂₀ aryloxy); 6 to 15 ring carbon atoms (i.e., C₆₋₁₅ aryloxy); and 6 to 10 ring carbon atoms (i.e., C₆₋₁₀ aryloxy). Examples of aryloxy moieties include, but are not limited to phenoxy, naphthoxy, and anthroxy.

As used herein, “arylene” refers to a divalent moiety of an aromatic compound or aryl wherein the ring atoms are only carbon atoms. Arylene is optionally substituted and can be monocyclic or polycyclic (e.g., bicyclic or tricyclic). Examples of arylene moieties include, but are not limited to, those having 6 to 20 ring carbon atoms (i.e., C₆₋₂₀ arylene); 6 to 15 ring carbon atoms (i.e., C₆₋₁₅ arylene); and 6 to 10 ring carbon atoms (i.e., C₆₋₁₀ arylene).

As used herein, “heteroalkyl” refers to an alkyl in which one or more carbon atoms are replaced by heteroatoms. As used herein, “heteroalkenyl” refers to an alkenyl in which one or more carbon atoms are replaced by heteroatoms. As used herein, “heteroalkynyl” refers to an alkynyl in which one or more carbon atoms are replaced by heteroatoms. Suitable heteroatoms include, but are not limited to, nitrogen (N), oxygen (O), and sulfur (S) atoms. Heteroalkyl, heteroalkenyl, and heteroalkynyl are optionally substituted. Examples of heteroalkyl moieties include, but are not limited to, aminoalkyl, hydroxyalkyl, sulfonylalkyl, and sulfinylalkyl. Examples of heteroalkyl moieties also include, but are not limited to, methylamino, methylsulfonyl, and methylsulfinyl.

As used herein, “heteroaryl” refers to a monovalent moiety that is a radical of an aromatic compound or aryl wherein the ring atoms contain carbon atoms and at least one oxygen, sulfur, nitrogen, or phosphorus atom. Examples of heteroaryl moieties include, but are not limited to, those having 5 to 20 ring atoms; 5 to 15 ring atoms; and 5 to 10 ring atoms. Heteroaryl is optionally substituted.

As used herein, “heteroarylene” refers to a divalent heteroaryl in which one or more ring atoms of the aromatic ring are replaced with an oxygen, sulfur, nitrogen, or phosphorus atom. Heteroarylene is optionally substituted.

As used herein, “heterocycloalkyl” refers to a cycloalkyl in which one or more carbon atoms are replaced with heteroatoms. Suitable heteroatoms include, but are not limited to, nitrogen, oxygen, and sulfur atoms. Heterocycloalkyl is optionally substituted. Examples of heterocycloalkyl moieties include, but are not limited to, morpholinyl, piperidinyl, tetrahydropyranyl, pyrrolidinyl, imidazolidinyl, oxazolidinyl, thiazolidinyl, dioxolanyl, dithiolanyl, oxanyl, or thianyl.

As used herein, “Lewis acid” refers to a molecule or ion that possesses an empty orbital and is capable of accepting or accepts an electron lone pair. Lewis acids include, but are not limited to, non-metal acids, metal acids, hard Lewis acids, and soft Lewis acids. Lewis acids include, but are not limited to, Lewis acids of aluminum, boron, iron, tin, titanium, magnesium, copper, antimony, phosphorus, silver, ytterbium, scandium, nickel, and zinc. Illustrative Lewis acids include, but are not limited to, AlBr₃, AlCl₃, BCl₃, boron trichloride methyl sulfide, BF₃, boron trifluoride methyl etherate, boron trifluoride methyl sulfide, boron trifluoride tetrahydrofuran, dicyclohexylboron trifluoromethanesulfonate, iron (III) bromide, iron (III) chloride, tin (IV) chloride, titanium (IV) chloride, titanium (IV) isopropoxide, Cu(OTf)₂, CuCl₂, CuBr₂, zinc chloride, alkylaluminum halides (RₙAlX₃₋ₙ, wherein R is hydrocarbyl or alkyl and X is a halide), Zn(OTf)₂, ZnCl₂, Yb(OTf)₃, Sc(OTf)₃, MgBr₂, NiCl₂, Sn(OTf)₂, Ni(OTf)₂, and Mg(OTf)₂.

As used herein, “N-containing heterocycloalkyl,” refers to a cycloalkyl in which one or more carbon atoms are replaced with heteroatoms and wherein at least one replacing heteroatom is a nitrogen atom. Suitable heteroatoms in addition to nitrogen include, but are not limited to, oxygen and sulfur atoms. N-containing heterocycloalkyl is optionally substituted. Examples of N-containing heterocycloalkyl moieties include, but are not limited to, morpholinyl, piperidinyl, pyrrolidinyl, imidazolidinyl, oxazolidinyl, or thiazolidinyl.

As used herein, “optionally substituted,” when used to describe a radical moiety or substituent, for example, optionally substituted alkyl, means that such moiety is optionally bonded to one or more substituents. Examples of such substituents include, but are not limited to, halo, cyano, nitro, amino, hydroxyl, optionally substituted haloalkyl, aminoalkyl, hydroxyalkyl, azido, epoxy, optionally substituted heteroaryl, optionally substituted heterocycloalkyl,

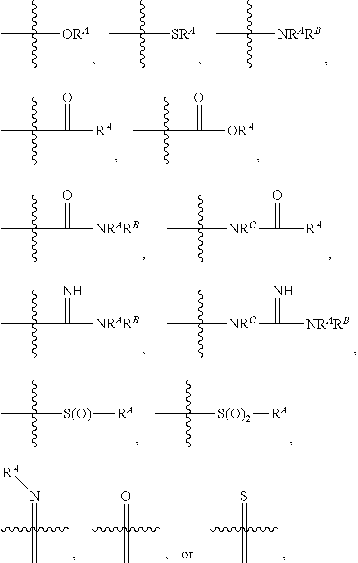

wherein R^{A}, R^{B}, and R^{C} are, independently at each occurrence, hydrogen, alkyl, alkenyl, alkynyl, aryl, alkylaryl, arylalkyl, heteroalkyl, heteroaryl, or heterocycloalkyl, or R^{A} and R^{B} together with the atoms to which they are bonded, form a saturated or unsaturated carbocyclic ring, wherein the ring is optionally substituted, and wherein one or more ring atoms is optionally replaced with a heteroatom. In certain embodiments, when a radical moiety is optionally substituted with an optionally substituted heteroaryl, optionally substituted heterocycloalkyl, or optionally substituted saturated or unsaturated carbocyclic ring, the substituents on the optionally substituted heteroaryl, optionally substituted heterocycloalkyl, or optionally substituted saturated or unsaturated carbocyclic ring, if they are substituted, are not substituted with substituents which are further optionally substituted with additional substituents. In some embodiments, when a group described herein is optionally substituted, the substituent bonded to the group is unsubstituted unless otherwise specified.

As used herein, “binding agent” refers to any molecule (e.g., protein, antibody, or fragment thereof) capable of binding with specificity to a given binding partner (e.g., antigen).

As used herein, “linker” refers to a divalent, trivalent, or multivalent moiety that covalently links, or is capable of covalently linking (e.g., via a reactive group at one terminus; and, in certain embodiments, an amino acid and/or a spacer at another terminus) the binding agent to one or more compounds described herein, for instance, payload compounds, enhancement groups or agents, and/or prodrug payload compounds. As used herein, “payloads” refer to tubulysins or tubulysin derivatives. As used herein, “prodrug payload compounds” or “prodrugs” refer to payloads that terminate with one or more amino acid residues, or another chemical residue, as described elsewhere herein. Thus, in certain embodiments, the linker can ultimately be cleaved to release payload compounds in the form of tubulysin derivatives. In other embodiments, the linker can ultimately be cleaved to release a prodrug payload compound in the form of a tubulysin derivative that retains one or more terminal amino acid residues. Such a prodrug payload compound can be further processed via accepted biological processes (e.g., amide bond hydrolysis) that ultimately produce payload compounds in the form of tubulysin payload compounds without terminal amino acid residues.

As used herein, “amide synthesis conditions” refers to reaction conditions suitable to effect the formation of an amide (e.g., by the reaction of a carboxylic acid, activated carboxylic acid, or acyl halide with an amine). In some examples, amide synthesis conditions refer to reaction conditions suitable to effect the formation of an amide bond between a carboxylic acid and an amine. In some of these examples, the carboxylic acid is first converted to an activated carboxylic acid before the activated carboxylic acid reacts with an amine to form an amide. Suitable conditions to effect the formation of an amide include, but are not limited to, those utilizing reagents to effect the reaction between a carboxylic acid and an amine including, but not limited to, dicyclohexylcarbodiimide (DCC), diisopropylcarbodiimide (DIC), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBrOP), O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), 1-[Bis(dimethylamino)methyl ene]-1H-1,2,3-triazolo[4,5-b]pyridinium 3-oxide hexafluorophosphate (HATU), N-ethoxycarbonyl-2-ethoxy-1,2-dihydroquinoline (EEDQ), N-ethyl-N′-(3-dimethylaminopropyl)carbodiimide (EDC), 2-chloro-1,3-dimethylimidazolidinium hexafluorophosphate (CIP), 2-chloro-4,6-dimethoxy-1,3,5-triazine (CDMT), and carbonyldiimidazole (CDI). In some examples, a carboxylic acid is first converted to an activated carboxylic ester before treating the activated carboxylic ester with an amine to form an amide bond. In certain embodiments, the carboxylic acid is treated with a reagent. The reagent activates the carboxylic acid by deprotonating the carboxylic acid and then forming a product complex with the deprotonated carboxylic acid as a result of nucleophilic attack by the deprotonated carboxylic acid onto the protonated reagent. The activated carboxylic esters for certain carboxylic acids are subsequently more susceptible to nucleophilic attack by an amine than the carboxylic acid is before activation. This results in amide bond formation. As such, the carboxylic acid is described as activated. Exemplary reagents include DCC and DIC.

As used herein, “regioisomer,” “regioisomers,” or “mixture of regioisomers” refers to the product(s) of 1,3-cycloadditions or strain-promoted alkyne-azide cycloadditions (SPAACs)— otherwise known as click reactions—that derive from suitable azides (e.g., —N₃, or —PEG-N₃ derivitized antibodies) treated with suitable alkynes. In certain embodiments, for example, regioisomers and mixtures of regioisomers are characterized by the click reaction products shown below

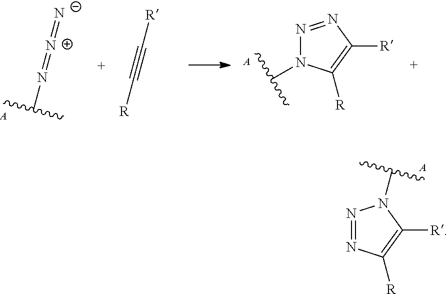

In certain embodiments, more than one suitable azide and more than one suitable alkyne can be utilized within a synthetic scheme en route to a product, where each pair of azide-alkyne can participate in one or more independent click reactions to generate a mixture of regioisomeric click reaction products. For example, a person of skill will recognize that a first suitable azide may independently react with a first suitable alkyne, and a second suitable azide may independently react with a second suitable alkyne, en route to a product, resulting in the generation of four possible click reaction regioisomers or a mixture of the four possible click reaction regioisomers.

As used herein, the term “residue” refers to the chemical moiety within a compound that remains after a chemical reaction. For example, the term “amino acid residue,” “N-alkyl amino acid residue,” or “N-terminal amino acid residue” refers to the product of an amide coupling or peptide coupling of an amino acid, N-alkyl amino acid, or N-terminal amino acid to a suitable coupling partner; wherein, for example, a water molecule is expelled after the amide or peptide coupling of the amino acid or the N-alkylamino acid, resulting in the product having the amino acid residue, N-alkyl amino acid residue, or N-terminal amino acid residue, incorporated therein. The term “amino acid” refers to naturally occurring and synthetic α, β, γ, or δ amino acids, and includes, but is not limited to, amino acids found in proteins, namely, glycine, alanine, valine, leucine, isoleucine, methionine, phenylalanine, tryptophan, proline, serine, threonine, cysteine, tyrosine, asparagine, glutamine, aspartate, glutamate, lysine, arginine, and histidine. In certain embodiments, the amino acid is in the L-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-isoleuccinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-argininyl or β-histidinyl. The term “amino acid derivative” refers to a group derivable from a naturally or non-naturally occurring amino acid, as described and exemplified herein. Amino acid derivatives are apparent to those of skill in the art and include, but are not limited to, ester, amino alcohol, amino aldehyde, amino lactone, and N-methyl derivatives of naturally and non-naturally occurring amino acids. In certain embodiments, an amino acid residue is

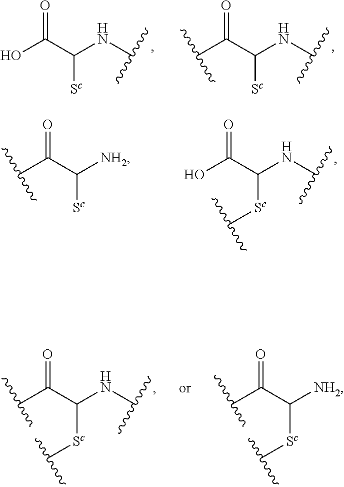

wherein S^{c} is a side chain of a naturally occurring or non-naturally occurring amino acid or a bond (e.g., hydrogen, as in glycine; —CH₂OH as in serine; —CH₂SH as in cysteine; —CH₂CH₂CH₂CH₂NH₂ as in lysine; —CH₂CH₂COOH as in glutamic acid; —CH₂CH₂C(O)NH₂ as in glutamine; or —CH₂C₆H₅OH as in tyrosine; and the like); and [symbol] represents the bonding to another chemical entity including, but not limited to, another amino acid residue or N-alkyl amino acid residue resulting in a peptide or peptide residue. In certain embodiments, S^{c} is selected from the group consisting of hydrogen, alkyl, heteroalkyl, arylalkyl, and heteroarylalkyl.

As used herein, “therapeutically effective amount” refers to an amount (e.g., of a compound described herein) that is sufficient to provide a therapeutic benefit to a patient in the treatment or management of a disease or disorder, or to delay or minimize one or more symptoms associated with the disease or disorder.

As used herein, “constitutional isomers” refers to compounds that have the same molecular formula, but different chemical structures resulting from the way the atoms are arranged. Exemplary constitutional isomers include n-propyl and isopropyl; n-butyl, sec-butyl, and tert-butyl; and n-pentyl, isopentyl, and neopentyl, and the like.

Certain groups, moieties, substituents, and atoms are depicted with a wiggly line that intersects a bond or bonds to indicate the atom through which the groups, moieties, substituents, and/or atoms are bonded. For example, a phenyl group that is substituted with a propyl group and depicted as

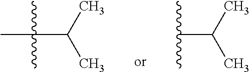

has the following structure

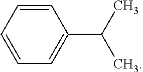

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which this disclosure pertains. For example, the group,

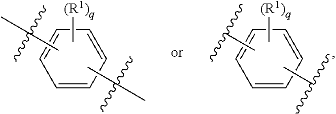

wherein subscript q is an integer from zero to four and in which the positions of substituent R′ are described generically (i.e., not directly attached to any vertex of the bond line structure (i.e., specific ring carbon atom)) includes the following, non-limiting examples of groups in which the substituent R¹ is bonded to a specific ring carbon atom

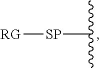

As used herein, the phrase “reactive linker,” or the abbreviation “RL” refers to a monovalent group that includes a reactive group (“RG”) and spacer group (“SP”), depicted, for example, as

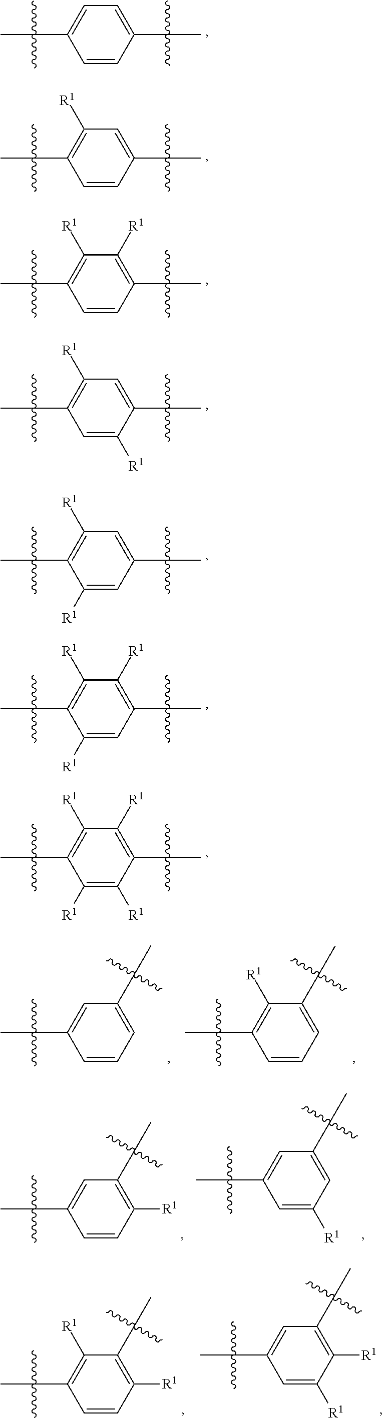

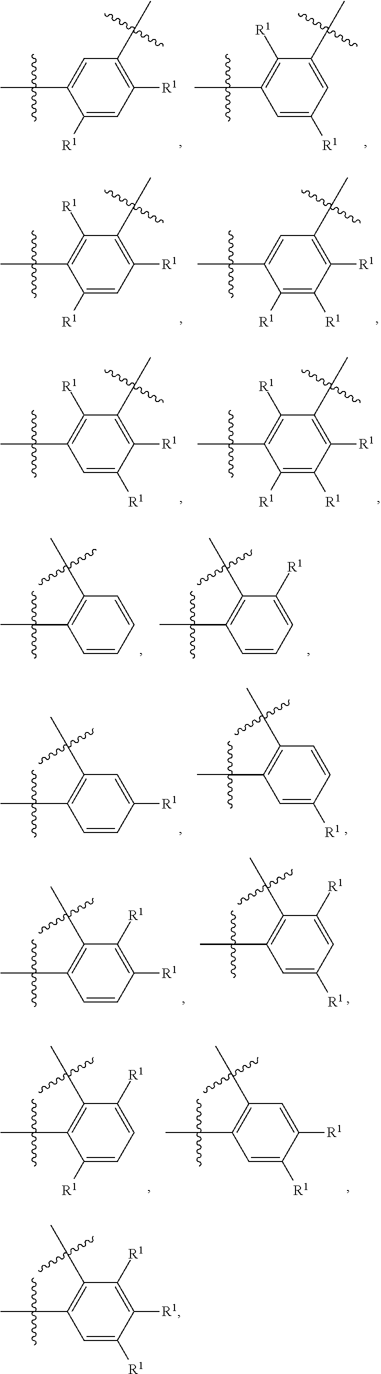

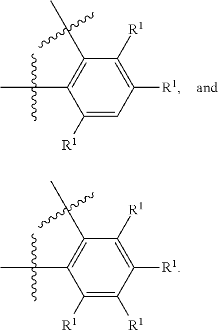

wherein RG is the reactive group and SP is the spacer group. As described herein, a reactive linker may include more than one reactive group and more than one spacer group. The spacer group is any divalent moiety that bridges the reactive group to another group, such as a payload or prodrug payload. The reactive linkers (RLs), together with the payloads or prodrug payloads to which they are bonded, provide intermediates (“linker-payloads” or LPs; or linker-prodrug payloads) useful as synthetic precursors for the preparation of the antibody-drug conjugates (ADCs) described herein. The reactive linker includes a reactive group, which is a functional group or moiety that is capable of reacting with a reactive portion of another group, for instance, an antibody or antigen-binding fragment thereof, modified antibody or antigen-binding fragment thereof, transglutaminase-modified antibody or antigen-binding fragment thereof, or an enhancement group. The moiety resulting from the reaction of the reactive group with the antibody or antigen-binding fragment thereof, modified antibody or antigen-binding fragment thereof, or transglutaminase-modified antibody or antigen-binding fragment thereof, together with the linking group include the “binding agent linker” (“BL”) portion of the conjugate described herein. In certain embodiments, the “reactive group” is a functional group or moiety (e.g., maleimide or N-hydroxysuccinimide (NETS) ester) that reacts with a cysteine or lysine residue of an antibody or antigen-binding fragment thereof. In certain embodiments, the “reactive group” is a functional group or moiety that is capable of undergoing a click chemistry reaction (see, e.g., click chemistry, Huisgen Proc. Chem. Soc. 1961, Wang et al. J. Am. Chem. Soc. 2003, and Agard et al. J. Am. Chem. Soc. 2004). In some embodiments of said click chemistry reaction, the reactive group is an alkyne that is capable of undergoing a 1,3-cycloaddition reaction with an azide. Such suitable reactive groups include, but are not limited to, strained alkynes, for example, those suitable for strain-promoted alkyne-azide cycloadditions (SPAAC), cycloalkynes, for example, cyclooctynes, benzannulated alkynes, and alkynes capable of undergoing 1,3-cycloaddition reactions with alkynes in the absence of copper catalysts. Suitable alkynes also include, but are not limited to, dibenzoazacyclooctyne or

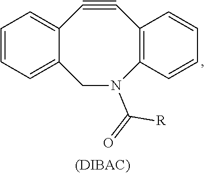

dibenzocyclooctyne or

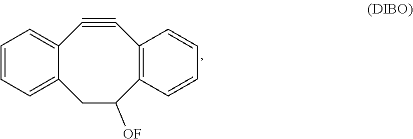

biarylazacyclooctynone or

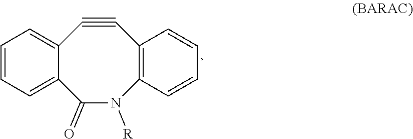

difluorinated cyclooctyne or

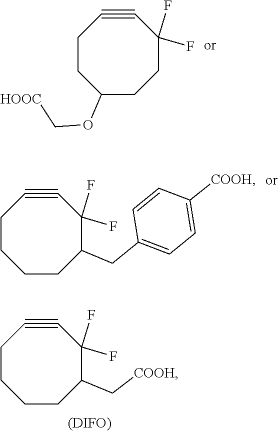

substituted, for example, fluorinated alkynes, aza-cycloalkynes, bicycle[6.1.0]nonyne or

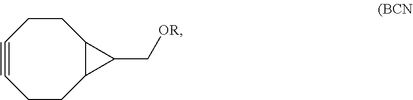

where R is alkyl, alkoxy, or acyl), and derivatives thereof. Particularly useful alkynes include

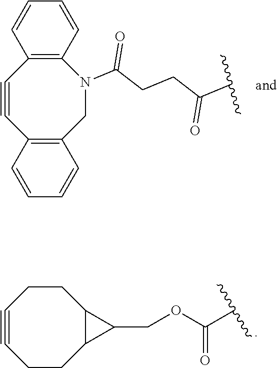 [SMILES: *C(=O)CCC(=O)N1Cc2ccccc2C#Cc2ccccc21 ; *C(=O)OCC1C2CCC#CCCC21]

Linker-payloads or linker-prodrug payloads including such reactive groups are useful for conjugating antibodies that have been functionalized with azido groups. As used herein, a “transglutaminase-modified antibody or antigen-binding fragment thereof” refers to an antibody or antigen-binding fragment thereof having one or more glutamine (Gln or Q) residues capable of reaction with a compound bearing a primary or secondary amino functional group in the presence of the enzyme transglutaminase. Such transglutaminase-modified antibodies or antigen-binding fragments thereof include antibodies or antigen-binding fragments thereof functionalized with azido-polyethylene glycol groups via transglutaminase-mediated coupling of an antibody or antigen-binding fragment thereof with a primary amine bearing the azido-polyethylene glycol moiety. In certain embodiments, such a transglutaminase-modified antibody or antigen-binding fragment thereof is derived by treating an antibody or antigen-binding fragment thereof having at least one glutamine residue, for example, heavy chain Gln295, with a compound bearing an amino group and an azide group, in the presence of the enzyme transglutaminase, as further described elsewhere herein.

In some examples, the reactive group is an alkyne, for example,

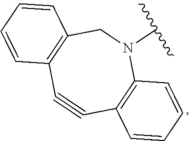 [SMILES: *N1Cc2ccccc2C#Cc2ccccc21]

which can react via click chemistry with an azide, for example,

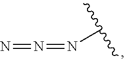

to form a click chemistry product, for example, regioisomeric

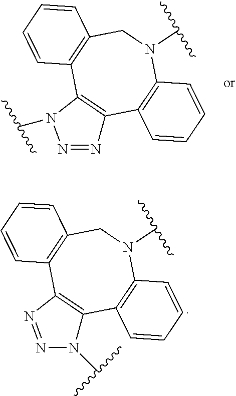 [SMILES: *N1Cc2ccccc2-c2c(nnn2*)-c2ccccc21 ; *N1Cc2ccccc2-c2nnn(*)c2-c2ccccc21]

In some examples, the reactive group reacts with an azide on a modified antibody or antigen binding fragment thereof. In some examples, the reactive group is an alkyne, for example,

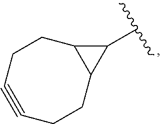 [SMILES: *C1C2CCC#CCCC12]

which can react via click chemistry with an azide, for example,

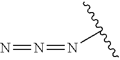

to form a click chemistry product, for example,

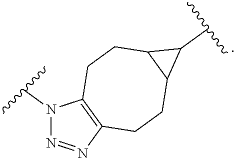 [SMILES: *C1C2CCc3nnn(*)c3CCC12]

In some examples, the reactive group reacts with an azide on a modified antibody or antigen binding fragment thereof. In some examples, the reactive group is an alkyne, for example,

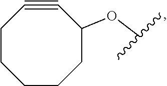 [SMILES: *OC1C#CCCCCC1]

which can react via click chemistry with an azide, for example,

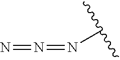

to form a click chemistry product, for example, regioisomeric

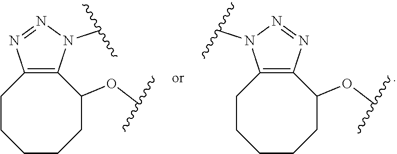 [SMILES: *OC1CCCCCc2nnn(*)c21 ; *OC1CCCCCc2c1nnn2*]

In some examples, the reactive group is an alkyne, for example,

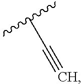 [SMILES: *C#C]

which can react via click chemistry with an azide, for example,

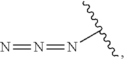

to form a click chemistry product, for example, regioisomeric

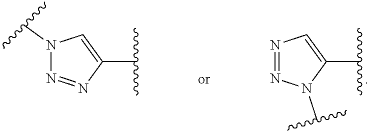 [SMILES: *c1cn(*)nn1 ; *c1cnnn1*]

In some examples, the reactive group is a functional group, for example,

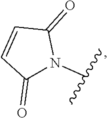 [SMILES: *N1C(=O)C=CC1=O]

which reacts with a cysteine residue on an antibody or antigen-binding fragment thereof to form a carbon-sulfur bond thereto, for example,

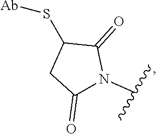

wherein Ab refers to an antibody or antigen-binding fragment thereof and S refers to the sulfur (S) atom on a cysteine residue through which the functional group bonds to the Ab. In some examples, the reactive group is a functional group, for example,

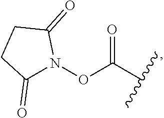 [SMILES: *C(=O)ON1C(=O)CCC1=O]

which reacts with a lysine residue on an antibody or antigen-binding fragment thereof to form an amide bond thereto, for example,

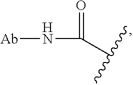

wherein Ab refers to an antibody or antigen-binding fragment thereof and —NH— refers to the —NH— atoms on a lysine side chain residue through which the functional group bonds to the Ab.

As used herein, the phrase “biodegradable moiety” refers to a moiety that degrades in vivo to non-toxic, biocompatible components which can be cleared from the body by ordinary biological processes. In some embodiments, a biodegradable moiety substantially or completely degrades in vivo over the course of about 90 days or less, about 60 days or less, or about 30 days or less, where the extent of degradation is based on percent mass loss of the biodegradable moiety, and wherein complete degradation corresponds to 100% mass loss. Exemplary biodegradable moieties include, without limitation, aliphatic polyesters such as poly(α-caprolactone) (PCL), poly(3-hydroxybutyrate) (PHB), poly(glycolic acid) (PGA), poly(lactic acid) (PLA) and its copolymers with glycolic acid (i.e., poly(D,L-lactide-coglycolide) (PLGA) (Vert M, Schwach G, Engel R, and Coudane J (1998) J Control Release 53(1-3):85-92; Jain R A (2000) Biomaterials 21(23):2475-2490; Uhrich K E, Cannizzaro S M, Langer R S, and Shakesheff K M (1999) Chemical Reviews 99(11):3181-3198; and Park T G (1995) Biomaterials 16(15):1123-1130, each of which are incorporated herein by reference in their entirety).

As used herein, the phrase “binding agent linker,” or “BL” refers to any divalent, trivalent, or multi-valent group or moiety that links, connects, or bonds a binding agent (e.g., an antibody or an antigen-binding fragment thereof) with a payload compound set forth herein (e.g., tubulysins) and, optionally, with one or more side chain compounds. Generally, suitable binding agent linkers for the antibody-drug conjugates described herein are those that are sufficiently stable to exploit the circulating half-life of the antibody-drug conjugates and, at the same time, capable of releasing the payload after antigen-mediated internalization of the conjugate. Linkers can be cleavable or non-cleavable. Cleavable linkers are linkers that are cleaved by intracellular metabolism following internalization, for example, cleavage via hydrolysis, reduction, or enzymatic reaction. Non-cleavable linkers are linkers that release an attached payload via lysosomal degradation of the antibody following internalization. Suitable linkers include, but are not limited to, acid-labile linkers, hydrolytically-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citrulline units, para-aminobenzyloxycarbonyl (PABC), and para-aminobenzyl (PAB) units. In some embodiments, the binding agent linker (BL) includes a moiety that is formed by the reaction of the reactive group (RG) of a reactive linker (RL) and reactive portion of the binding agent, for example, antibody, modified antibody, or antigen binding fragment thereof.

In some examples, the BL includes the following moiety

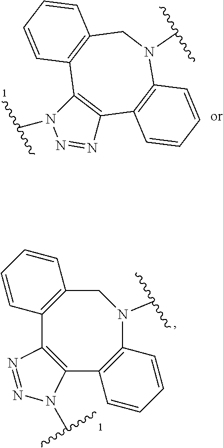 [SMILES: *N1Cc2ccccc2-c2c(nnn2*)-c2ccccc21 ; *N1Cc2ccccc2-c2nnn(*)c2-c2ccccc21]

wherein [symbol] the bond to the binding agent. In some examples, the BL includes the following moiety

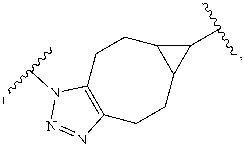

wherein [symbol] is the bond to the binding agent. In some examples, the BL includes the following moiety

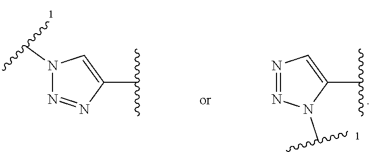

wherein [symbol] is the bond to the binding agent. In some examples, the BL includes the following moiety

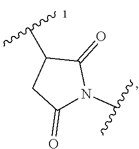

wherein [symbol] is the bond to the cysteine of the antibody or antigen-binding fragment thereof. In some examples, the BL includes the following moiety

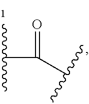

wherein [symbol] is the bond to the lysine of the antibody or antigen-binding fragment thereof.

As applied to polypeptides, the phrase “substantial similarity” or “substantially similar” means that two peptide sequences, when optimally aligned, such as by the programs GAP or BESTFIT using default gap weights, share at least 95% sequence identity, or at least 98% or 99% sequence identity. Sequence similarity may also be determined using the BLAST algorithm, described in Altschul et al. J. Mol. Biol. 215: 403-10 (using the published default settings), or available at blast.ncbi.nlm.nih.gov/Blast.cgi. In certain embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Methods for making this adjustment are well-known to those of skill in the art. See, for example, Pearson (1994) Methods Mol. Biol. 24: 307-331. Examples of groups of amino acids that have side chains with similar chemical properties include (1) aliphatic side chains: glycine, alanine, valine, leucine, and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate; and (7) sulfur-containing side chains: cysteine and methionine. Particularly useful conservative amino acids substitution groups include valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

As used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]-[(S)-compound])/([(R)-compound]+[(S)-compound])×100, where the (R)-compound >(9-compound; or % ee=([(9-compound]-[(R)-compound])/([(S)-compound]+[(R)-compound])×100, where the (9-compound >(R)-compound. In addition, as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

### Compounds, Payloads, or Prodrug Payloads

Provided herein are compounds, biologically active compounds, or payloads. Without being bound by any particular theory of operation, the compounds include tubulysins and derivatives thereof, for example, prodrugs thereof. The terms or phrases “compounds,” “biologically active compounds,” “prodrugs,” “prodrug payloads,” and “payloads” are used interchangeably throughout this disclosure.

In certain embodiments, the biologically active compound (D*) or residue thereof includes, for example, amino, hydroxyl, carboxylic acid, and/or amide functionality (e.g., D*-NH₂ or D*-NH—R; D*-OH or D*-O—R; D*-COOH or D*-C(O)O—R; and/or D*-CONH₂, D*-CONH—R, or D*-NHC(O)—R). In certain embodiments herein, for example and convenience, a heterocyclic nitrogen, R₂, R³, R⁶, and/or R⁷ represents the amino, hydroxyl, carboxylic acid, and amide functional groups within the biologically active compounds described herein, as would be appreciated by a person of skill in the art. Alternatively stated, a person of skill would recognize that a heterocyclic nitrogen, R₂, R³, R⁶, and/or R⁷ may be part of the biologically active compounds described herein (e.g., D*), and may be used as a functional group for conjugation purposes. In one embodiment, the hydroxyl functionality is a primary hydroxyl moiety (e.g., D*-CH₂OH or D*-CH₂O—R; or D*-C(O)CH₂OH or D*-C(O)CH₂O—R). In another embodiment, the hydroxyl functionality is a secondary hydroxyl moiety (e.g., D*-CH(OH)R or D*-CH(O—R)R; or D*-C(O)CH(R)(OH) or D*-C(O)CH(R)(O—R)). In another embodiment, the hydroxyl functionality is a tertiary hydroxyl moiety (e.g., D*-C(R₁)(R₂)(OH) or D*-C(R₁)(R₂)(O—R); or D*-C(O)C(R₁)(R₂)(OH) or D*-C(O)C(R₁)(R₂)(O—R)). In certain embodiments, the biologically active compound (D*) or residue thereof includes amino functionality (e.g., D*-NR² or D*-N(R)—R). In one embodiment, the amino functionality is a primary amino moiety (e.g., D*-CH₂NR² or D*-CH₂N(R)—R; or D*-C(O)CH₂NR² or D*-C(O)CH₂N(R)—R). In another embodiment, the amino functionality is a secondary amino moiety (e.g., D*-CH(NR²)R or D*-CH(NR—R)R; or D*-C(O)CH(R)(NR²) or D*-C(O)CH(R)(NR—R)). In another embodiment, the amino functionality is a tertiary amino moiety (e.g., D*-C(R₁)(R₂)(NR₂) or D*-C(R₁)(R₂)(N(R)—R); or D*-C(O)C(R₁)(R₂)(NR²) or D*-C(O)C(R₁)(R₂)(N(R)—R)). In another embodiment, the amino functionality is quaternary, as would be appreciated by a person of skill in the art. In another embodiment, the D* including the amino functionality is an aryl amine (e.g., D*-Ar—NR², D*-Ar—N(R)—R. Those of skill will recognize that each functional group in the previous sentences can be part of the biologically active compound D* and simultaneously be depicted in the formula for clarity, convenience, and/or emphasis. In another embodiment, the D* including the hydroxyl functionality is an aryl hydroxyl or phenolic hydroxyl (e.g., D*-Ar—OH, D*-Ar—O—R. In another embodiment, D* including the amide functionality is a tubulysin prodrug residue resulting from the reaction of a tubulysin compound or derivative, for example at R₂, R³, R⁴, R⁶, and/or R⁷ described herein, and an amino acid compound also described herein. For example, in certain embodiments, D*-NHC(O)C(S^{c})(H)NH₂ represents a tubulysin prodrug bearing an N-terminal amino acid residue, wherein Sc represents an amino acid side chain. By way of further example, in certain embodiments, D*-NH[C(O)C(S^{c})(H)NH]ₐₐC(O)C(S^{c})(H)NH₂ represents a tubulysin prodrug bearing an N-terminal peptide residue, wherein Sc representss an amino acid side chain and aa is an integer from one to one hundred. In certain embodiments, aa is one. In certain embodiments, aa is two. In certain embodiments, aa is three. In certain embodiments, aa is four. In certain embodiments, aa is five. As used herein, “amino acid side chain” refers to the additional chemical moiety on the same carbon that bears a primary or secondary amine and a carboxylic acid of an amino acid. As would be appreciated by a person of skill in the art, there are twenty-one “standard” amino acids. Exemplary “standard” amino acids include, without limitation, alanine, serine, proline, arginine, and aspartic acid. Other amino acids include, cysteine, selenocysteine, and glycine (e.g., wherein the additional chemical moiety on the same carbon that bears the primary amine and carboxylic acid of glycine is hydrogen). Exemplary amino acid side chains include, without limitation, methyl (i.e., alanine), sec-buytl (i.e., isoleucine), iso-butyl (i.e., leucine), —CH₂CH₂SCH₃ (i.e., methionine), —CH₂Ph (i.e., phenylalanine),

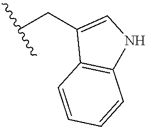 [SMILES: *Cc1c[nH]c2ccccc12]

(i.e., tryptophan),

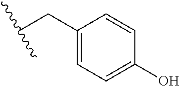 [SMILES: *Cc1ccc(O)cc1]

(i.e., tyrosine), iso-propyl (i.e., valine), hydroxymethyl (i.e., serine), —CH(OH)CH₃ (i.e., threonine), —CH₂C(O)NH₂ (i.e., asparagine), —CH₂CH₂C(O)NH₂ (i.e., glutamine), —CH₂SH (i.e., cysteine), —CH₂SeH (i.e., selenocysteine), —CH₂NH₂ (i.e., glycine), propylene or —CH₂CH₂CH₂— (i.e., proline), —CH₂CH₂CH₂NHC(═NH)NH₂ (i.e., arginine),

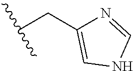 [SMILES: *Cc1c[nH]cn1]

(i.e., histidine), —CH₂CH₂CH₂CH₂NH₂ (i.e., lysine), —CH₂COOH (i.e., aspartic acid), and —CH₂CH₂COOH (i.e., glutamic acid).

In certain embodiments, the biologically active compound (D*) including amide functionality (D*-NHC(O)—R), for example at R³, is a prodrug compound of Formula Ia

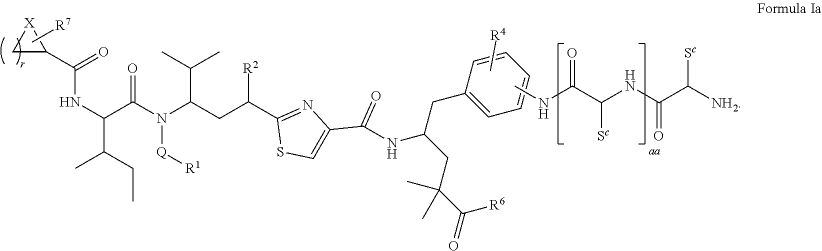

In certain embodiments, prodrug Formula Iaa

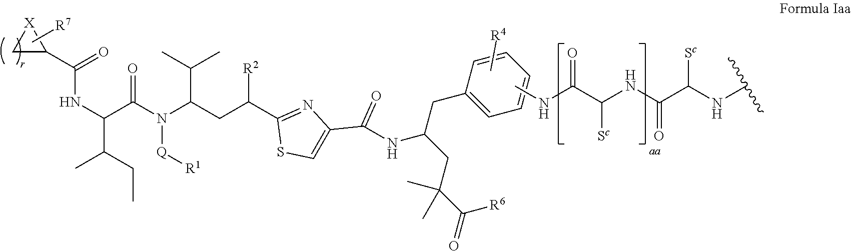

can be linked to a linker or binding agent, as described elsewhere herein, wherein [symbol] indicates an attachment to the linker, and/or binding agent, as described elsewhere herein.

In certain embodiments, the compounds can be delivered to cells as part of a conjugate. In certain embodiments, the compounds are capable of carrying out any activity of tubulysin or a tubulysin derivative at or in a target, for instance, a target cell. Certain compounds can have one or more additional activities. In certain embodiments, the compounds are capable of modulating the activity of a folate receptor, a somatostatin receptor, and/or a bombesin receptor.

### Compounds, Payloads, or Prodrug Payloads—Q is Carbon

In certain embodiments, set forth herein is a compound having the structure of Formula I or (I), wherein r is three.

In certain embodiments, set forth herein is a compound having the structure of Formula I, wherein r is four.

In certain embodiments of Formula I above, useful R² groups include —O—C(O)—NH—CH₂—CH(OH)—CH₂OH, —N—C(O)CH₃, —O—CH₂CH₃, —O—(CH₂)₃—OH, —O—C(O)CH₃, —O—C(O)—NH—(CH₂)₂—OH, —O—C(O)—NH—(CH₂CH₂O)₃—CH₂NH—C(O)CH₂NH₂, or —O—C(O)—NH—(CH₂CH₂O)₂—(CH₂)₂OH. In certain embodiments, R² is —O—C(O)—NH—CH₂—CH(OH)—CH₂OH. In certain embodiments, R² is —N—C(O)CH₃. In certain embodiments, R² is —O—CH₂CH₃. In certain embodiments, R² is —O—(CH₂)₃—OH. In certain embodiments, R² is —O—C(O)CH₃. In certain embodiments, R² is —O—C(O)—NH—(CH₂)₂—OH. In certain embodiments, R² is —O—C(O)—NH—(CH₂CH₂O)₃—CH₂NH—C(O)CH₂NH₂. In certain embodiments, R² is —O—C(O)—NH—(CH₂CH₂O)₂—(CH₂)₂OH.

In certain embodiments of Formula I above, useful R³ groups include —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂. In certain embodiments, R³ is —OH. In certain embodiments, R³ is —NH₂. In certain embodiments, R³ is —NH—(CH₂)₂OH. In certain embodiments, R³ is —NH—CH₂—C(O)—NH₂. In certain embodiments, R³ is —NH—CH₂—C(O)—OH. In certain embodiments, R³ is —NH—C(O)—CH₂NH₂. In certain embodiments, R³ is —NH—[(CH₂)₂OH]—C(O)—NH₂. In certain embodiments, R³ is —NH—CH₂—(CH₂O)₂—(CH₂)₂—NH₂. In certain embodiments, R³ is —N(CH₂CH₂OH)(C(O)CH₂NH₂). In certain embodiments, R³ is —NH—CH₂—C(O)—NH—(CH₂CH₂O)₂—(CH₂)₂NH₂.

In certain embodiments of Formula I above, useful R⁴ groups include hydrogen or —F. In certain embodiments, R⁴ is hydrogen. In certain embodiments, R⁴ is —F.

In certain embodiments, set forth herein is a compound having the structure of Formula I

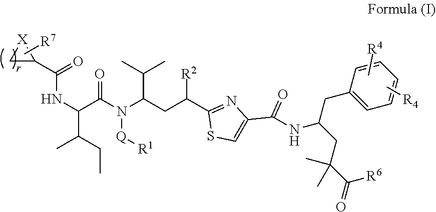

or a pharmaceutically acceptable salt thereof, wherein X is —O— or —NR⁵. In Formula I, in certain embodiments, X is —O—. In Formula I, in certain embodiments X is —NR⁵. In certain embodiments, useful R⁵ groups include hydrogen, —CH₃, —(CH₂)₂—OH, —(CH₂)₂—NH₂, —CH₂—C(O)—OH, —(CH₂)₂—(CH₂CH₂—O)₂—(CH₂)₂—NH₂, or —C(O)—CH₂—NH₂. In certain embodiments, R⁵ is hydrogen. In certain embodiments, R⁵ is —CH₃. In certain embodiments, R⁵ is —(CH₂)₂—OH. In certain embodiments, R⁵ is —(CH₂)₂—NH₂. In certain embodiments, R⁵ is —CH₂—C(O)—OH. In certain embodiments, R⁵ is —(CH₂)₂—O—(CH₂)₂—NH₂. In certain embodiments, R⁵ is —(CH₂CH₂—O)₂—(CH₂)₂—NH₂. In certain embodiments, R⁵ is —C(O)—CH₂—NH₂. In Formula I, in certain embodiments, Q is —CH₂— or —O—. In Formula I, in certain embodiments, Q is —CH₂—. In Formula I, in certain embodiments, Q is —O—. In Formula I, in certain embodiments, useful R⁴ groups include —C₅ alkyl or —C₅ alkynyl. In certain embodiments, R⁴ is −C₅ alkyl. In certain embodiments, R⁴ is —C₅ alkynyl. In Formula I, in certain embodiments, useful R² groups include —O—C(O)—NH—CH₂—CH(OH)—CH₂OH, —N—C(O)CH₃, —O—CH₂CH₃, —O—(CH₂)₃—OH, —O—C(O)CH₃, —O—C(O)—NH—(CH₂)₂—OH, or —O—C(O)—NH—(CH₂CH₂O)₂—(CH₂)₂OH. In certain embodiments, R² is —O—C(O)—NH—CH₂—CH(OH)—CH₂OH. In certain embodiments, R² is —N—C(O)CH₃. In certain embodiments, R² is —O—CH₂CH₃. In certain embodiments, R² is —O—(CH₂)₃—OH. In certain embodiments, R² is —O—C(O)CH₃. In certain embodiments, R² is —O—C(O)—NH—(CH₂)₂—OH. In certain embodiments, R² is —O—C(O)—NH—(CH₂CH₂O)₂—(CH₂)₂OH. In Formula I, in certain embodiments, useful R³ groups include —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, or —NH—CH₂—C(O)—NH—(CH₂CH₂O)₂—(CH₂)₂NH₂. In certain embodiments, R³ is —OH. In certain embodiments, R³ is —NH₂. In certain embodiments, R³ is —NH—(CH₂)₂OH. In certain embodiments, R³ is —NH—CH₂—C(O)—NH₂. In certain embodiments, R³ is —NH—CH₂—C(O)—OH. In certain embodiments, R³ is —NH—C(O)—CH₂NH₂. In certain embodiments, R³ is —NH—[(CH₂)₂OH]—C(O)—NH₂. In certain embodiments, R³ is —NH—CH₂—(CH₂O)₂—(CH₂)₂—NH₂. In certain embodiments, R³ is —NH—CH₂—C(O)—NH—(CH₂CH₂O)₂—(CH₂)₂NH₂. In certain embodiments of Formula I, useful R⁴ groups include hydrogen or —F. In certain embodiments, R⁴ is hydrogen. In certain embodiments, R⁴ is —F. In certain embodiments of Formula I, R⁷ when present is —CH₃. In certain embodiments of Formula I, useful R⁶ groups include —OH or —NH—C(O)OH. In certain embodiments, R⁶ is —OH. In certain embodiments, R⁶ is —NH—C(O)OH. In one embodiment, r is three. In one embodiment, r is four.

In certain embodiments, provided herein are compounds according to Formula I, selected from Table P.

TABLE P

| ID No./ | |
|---|---|
| Com- | |
| pound | |
| No. | Structure |
| PA1 | 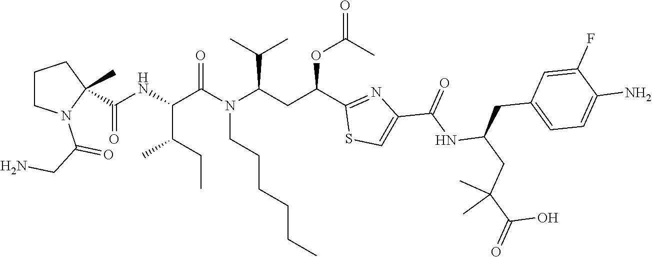 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| PA2 | 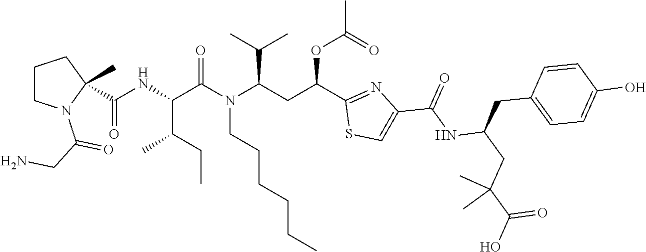 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| PA3 | 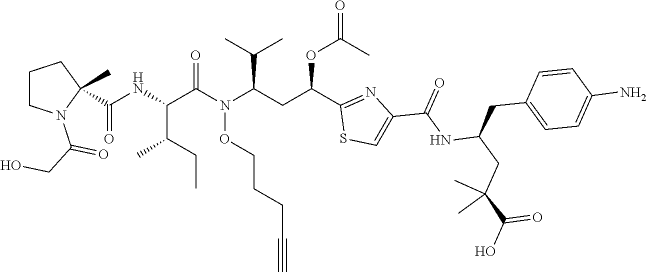 |
| PA4 | 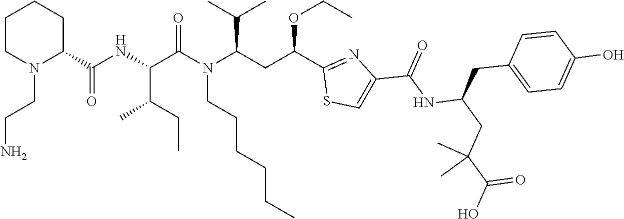 [SMILES: CCCCCCN(C(=O)[C@@H](NC(=O)[C@H]1CCCCN1CCN)[C@@H](C)CC)[C@H](C[C@@H](OCC)c1nc(C(=O)N[C@@H](Cc2ccc(O)cc2)CC(C)(C)C(=O)O)cs1)C(C)C] |
| PA5 | 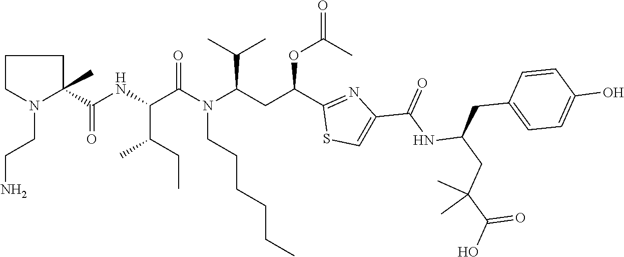 [SMILES: CCCCCCN(C(=O)[C@@H](NC(=O)[C@@]1(C)CCCN1CCN)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| PA6 | 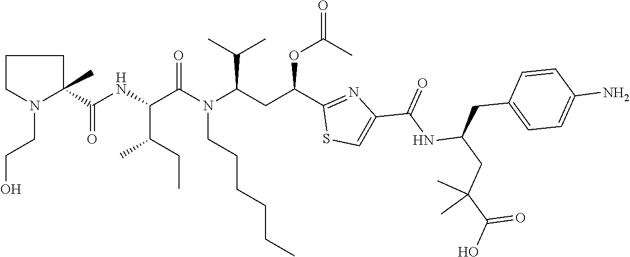 [SMILES: CCCCCCN(C(=O)[C@@H](NC(=O)[C@@]1(C)CCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| PA7 | 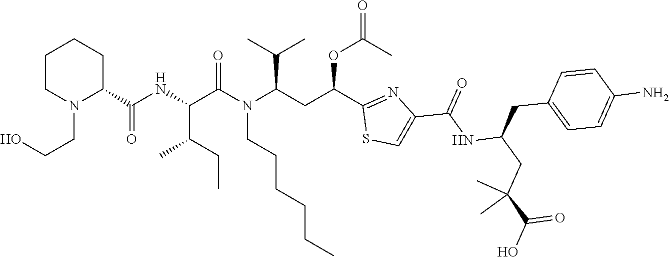 |
| PA8 | 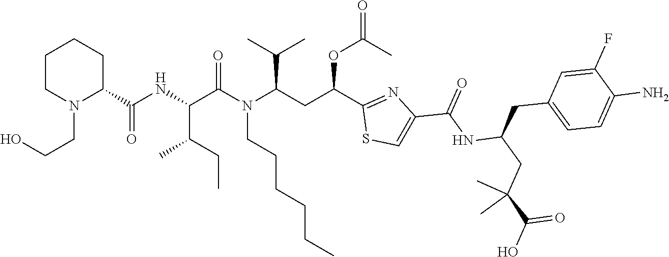 |
| PA9 | 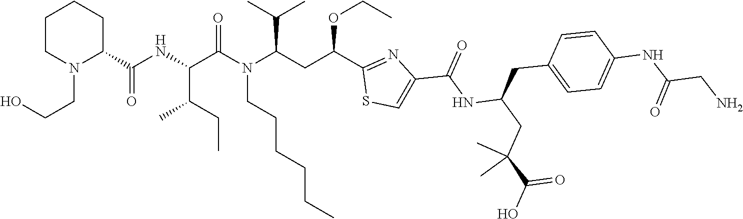 |
| PA10 | 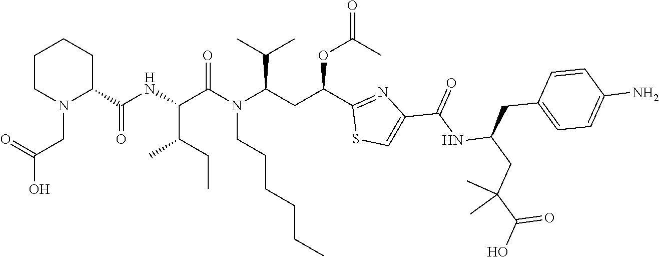 [SMILES: CCCCCCN(C(=O)[C@@H](NC(=O)[C@H]1CCCCN1CC(=O)O)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| PA11 | 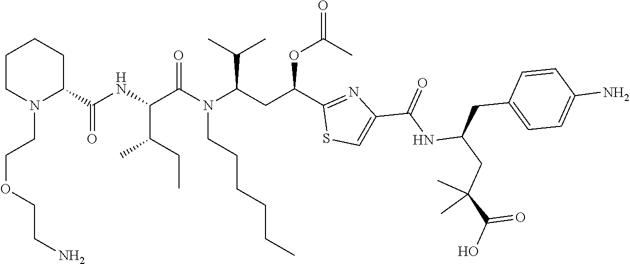 |
| PA12 | 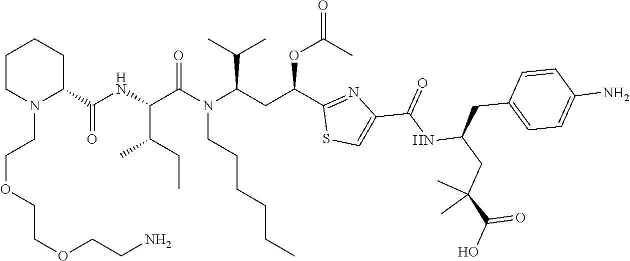 |
| PA13 | 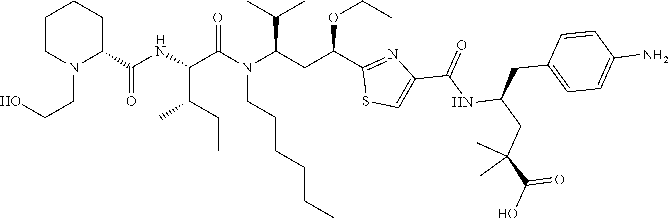 |
| PA14 | 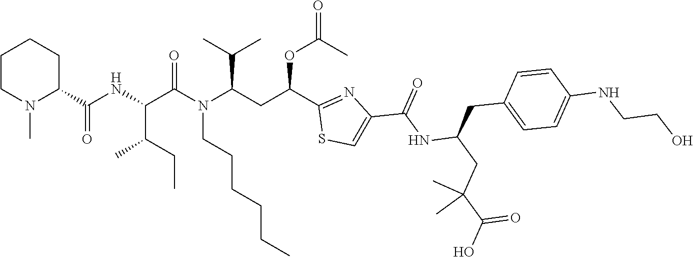 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCO)cc2)CC(C)(C)C(=O)O)cs1)C(C)C] |
| PA15 | 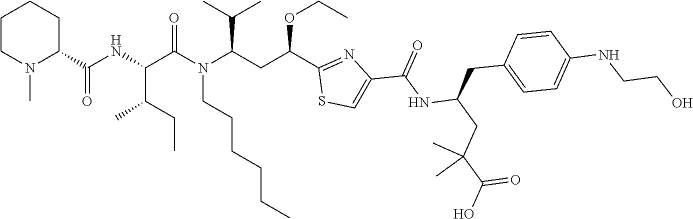 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCO)cc2)CC(C)(C)C(=O)O)cs1)C(C)C] |
| PA16 | 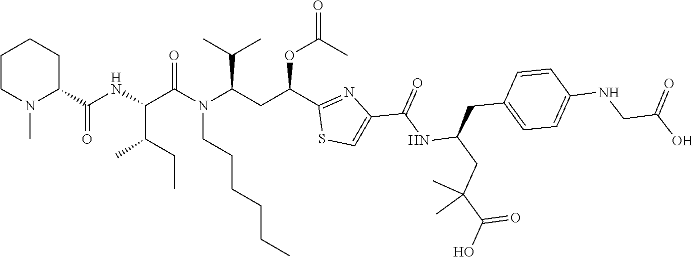 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C] |
| PA17 | 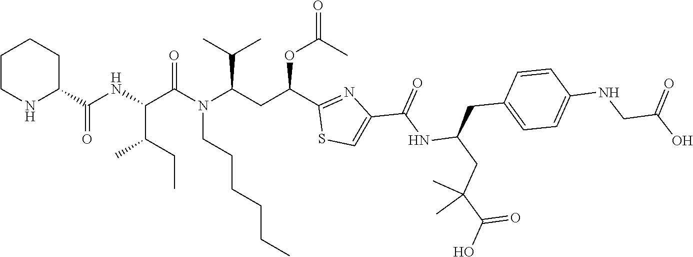 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C] |
| PA18 | 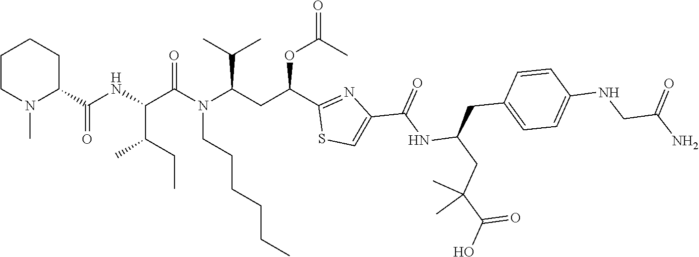 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(N)=O)cc2)CC(C)(C)C(=O)O)cs1)C(C)C] |
| PA19 | 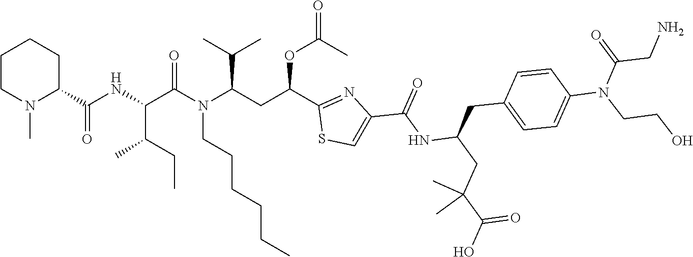 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N(CCO)C(=O)CN)cc2)CC(C)(C)C(=O)O)cs1)C(C)C] |
| PA20 | 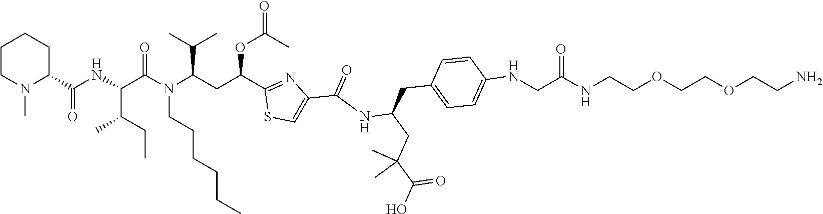 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCN)cc2)CC(C)(C)C(=O)O)cs1)C(C)C] |
| PA21 | 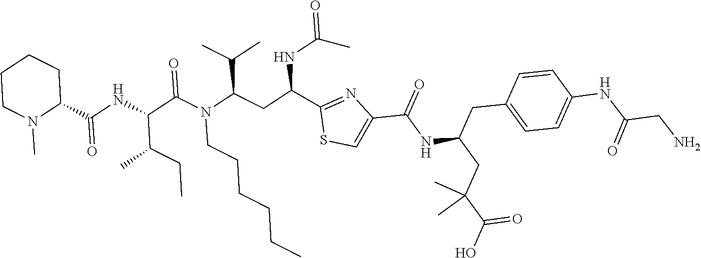 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(NC(=O)CN)cc2)CC(C)(C)C(=O)O)cs1)C(C)C] |
| PA22 | 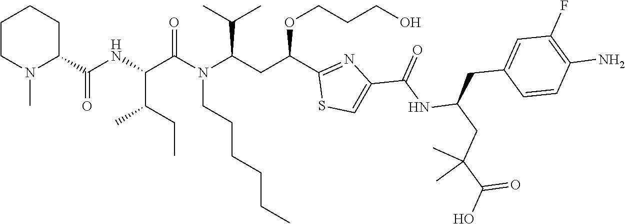 [SMILES: CCCCCCN(C(=O)[C@@H](NC(=O)[C@H]1CCCCN1C)[C@@H](C)CC)[C@H](C[C@@H](OCCCO)c1nc(C(=O)N[C@@H](Cc2ccc(N)c(F)c2)CC(C)(C)C(=O)O)cs1)C(C)C] |
| PA23 | 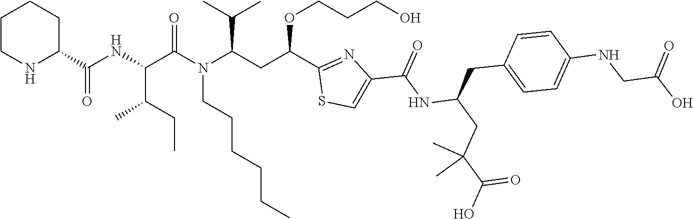 [SMILES: CCCCCCN(C(=O)[C@@H](NC(=O)[C@H]1CCCCN1)[C@@H](C)CC)[C@H](C[C@@H](OCCCO)c1nc(C(=O)N[C@@H](Cc2ccc(NCC(=O)O)cc2)CC(C)(C)C(=O)O)cs1)C(C)C] |
| PA24 | 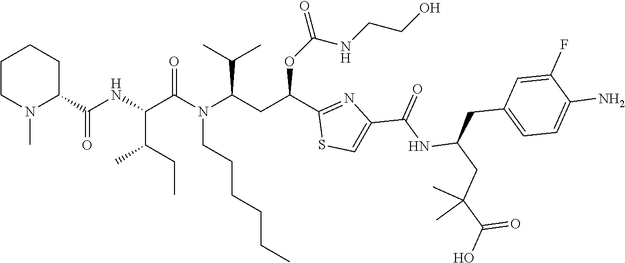 [SMILES: CCCCCCN(C(=O)[C@@H](NC(=O)[C@H]1CCCCN1C)[C@@H](C)CC)[C@H](C[C@@H](OC(=O)NCCO)c1nc(C(=O)N[C@@H](Cc2ccc(N)c(F)c2)CC(C)(C)C(=O)O)cs1)C(C)C] |
| PA25 | 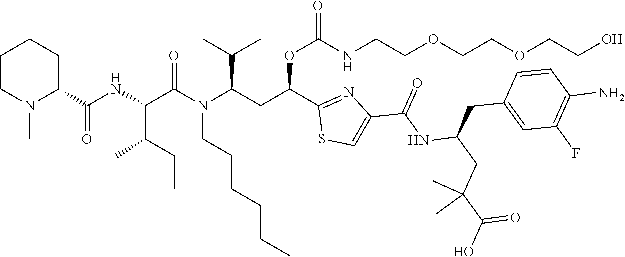 [SMILES: CCCCCCN(C(=O)[C@@H](NC(=O)[C@H]1CCCCN1C)[C@@H](C)CC)[C@H](C[C@@H](OC(=O)NCCOCCOCCO)c1nc(C(=O)N[C@@H](Cc2ccc(N)c(F)c2)CC(C)(C)C(=O)O)cs1)C(C)C] |
| PA26 | 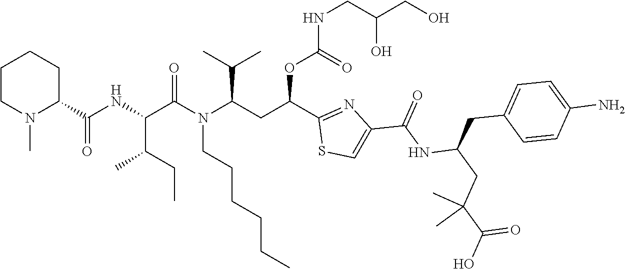 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(N)cc2)CC(C)(C)C(=O)O)cs1)C(C)C] |
| PA27 | 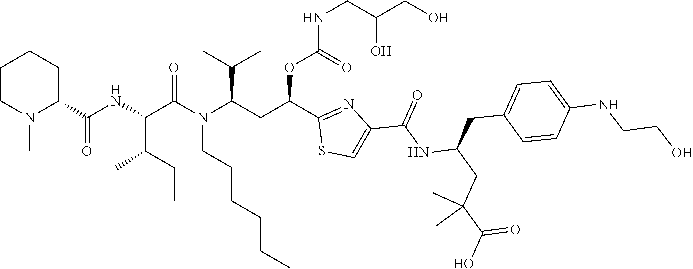 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(NCCO)cc2)CC(C)(C)C(=O)O)cs1)C(C)C] |
| PA28 | 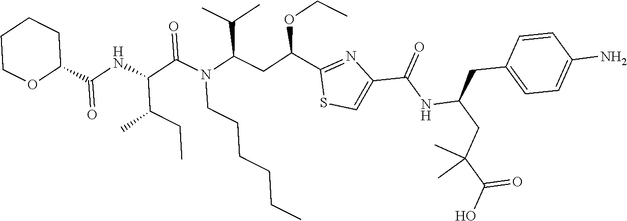 [SMILES: CCCCCCN(C(=O)[C@@H](NC(=O)[C@H]1CCCCO1)[C@@H](C)CC)[C@H](C[C@@H](OCC)c1nc(C(=O)N[C@@H](Cc2ccc(N)cc2)CC(C)(C)C(=O)O)cs1)C(C)C] |
| PA29 | 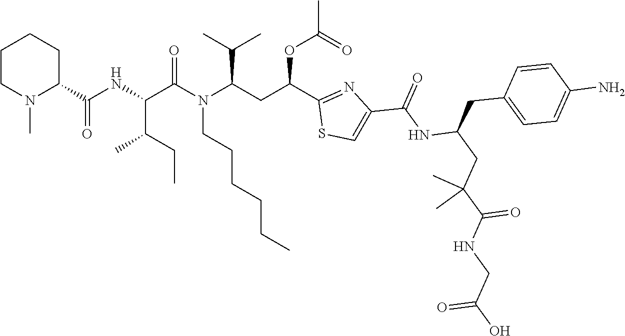 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)NCC(=O)O)cs1)C(C)C] |
| PA30 | 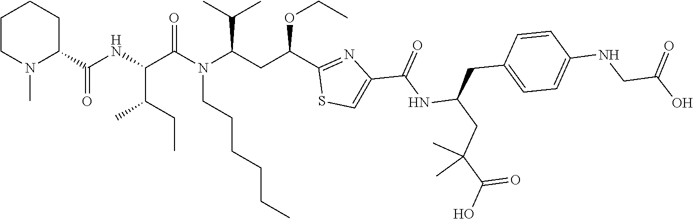 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCC(=O)O)cc2)CC(C)(C)C(=O)O)cs1)C(C)C] |

In certain embodiments, set forth herein is a compound having the structure of Formula II

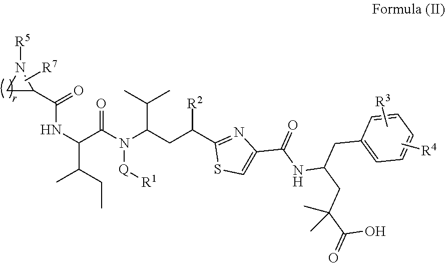

or a pharmaceutically acceptable salt thereof. In certain embodiments, R¹, R², R³, R⁴, R⁵, R⁷, and r are as described in the context of Formula I, above. In certain embodiments, R⁵ is —(CH₂)₂—OH or —(CH₂)₂—NH₂. In certain embodiments, R⁵ is —(CH₂)₂—OH. In certain embodiments, R⁵ is —(CH₂)₂—NH₂.

In certain embodiments, provided herein are compounds according to Formula II, selected from the group consisting of

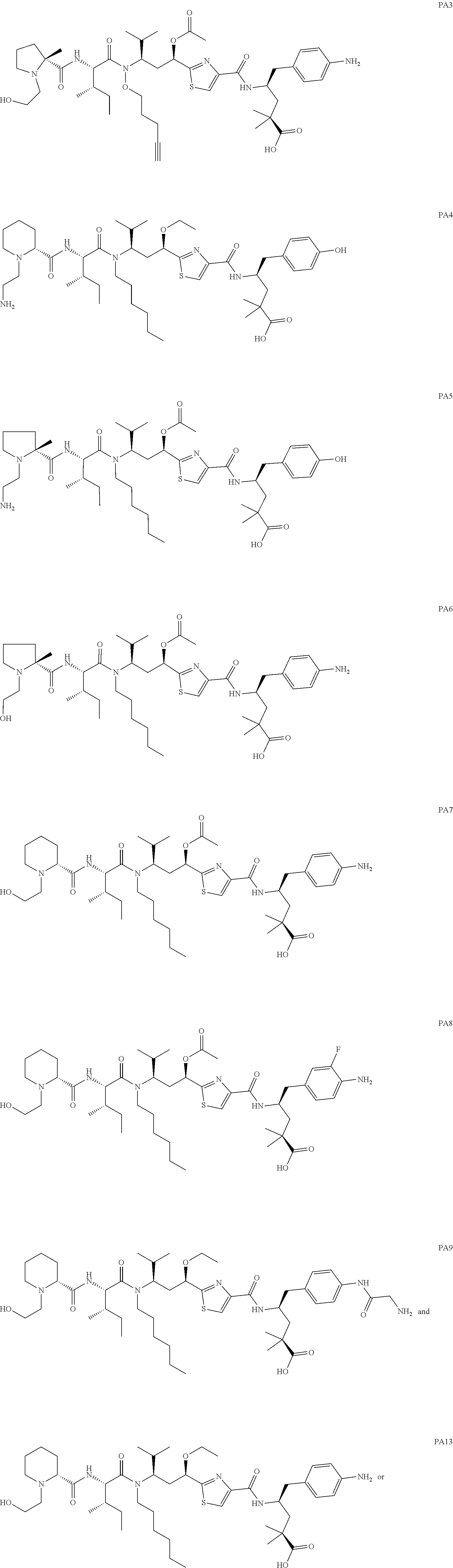

a pharmaceutically acceptable salt thereof.

In Formula II, in certain embodiments, R⁵ is —CH₂—C(O)—OH or —C(O)—CH₂—NH₂. In certain embodiments, R⁵ is —CH₂—C(O)—OH. In certain embodiments, R⁵ is —C(O)—CH₂—NH₂.

In certain embodiments, provided herein are compounds according to Formula II, selected from the group consisting of

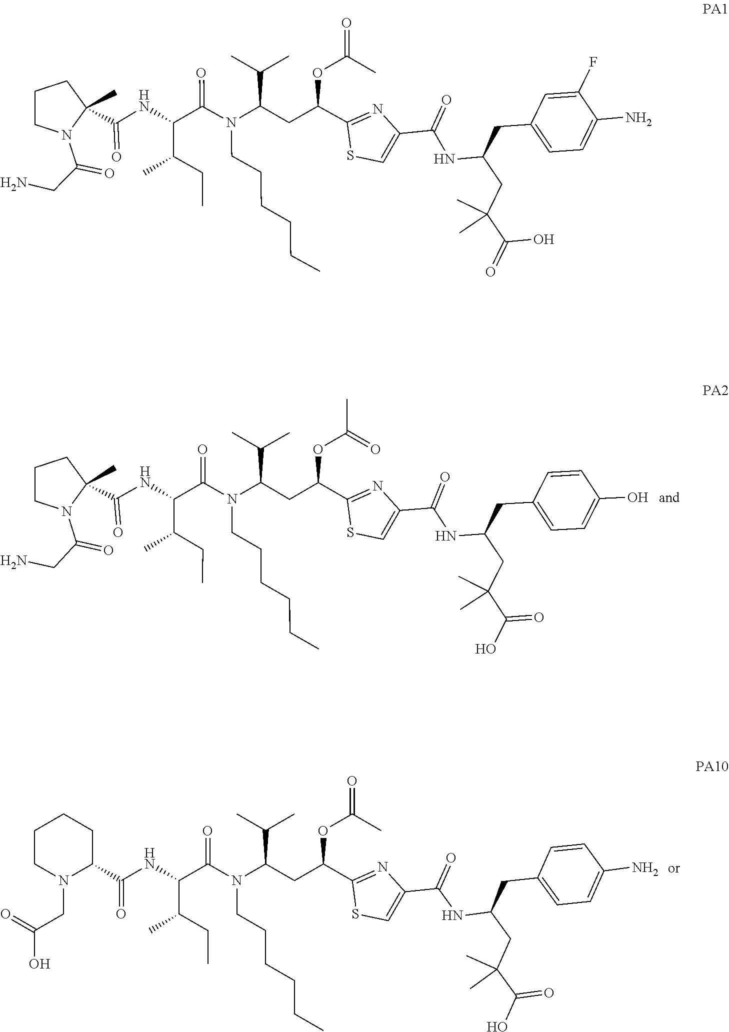 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C ; CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(O)cc2)CC(C)(C)C(=O)O)cs1)C(C)C ; CCCCCCN(C(=O)[C@@H](NC(=O)[C@H]1CCCCN1CC(=O)O)[C@@H](C)CC)[C@H](C[C@@H](OC(C)=O)c1nc(C(=O)N[C@@H](Cc2ccc(N)cc2)CC(C)(C)C(=O)O)cs1)C(C)C]

a pharmaceutically acceptable salt thereof.

In certain embodiments, set forth herein is a compound having the structure of Formula III

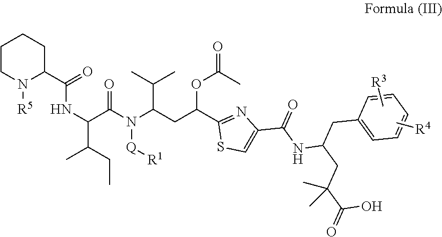

or a pharmaceutically acceptable salt thereof. In certain embodiments, R¹, R³, R⁴, and R⁵ are as described in the context of Formula I, above. In certain embodiments, R⁵ is —(CH₂)₂—O—(CH₂)₂—NH₂ or —(CH₂CH₂—O)₂—(CH₂)₂—NH₂. In certain embodiments, R⁵ is —(CH₂)₂—O—(CH₂)₂—NH₂. In certain embodiments, R⁵ is —(CH₂CH₂—O)₂—(CH₂)₂—NH₂.

In certain embodiments, provided herein are compounds according to Formula III, selected from the group consisting of

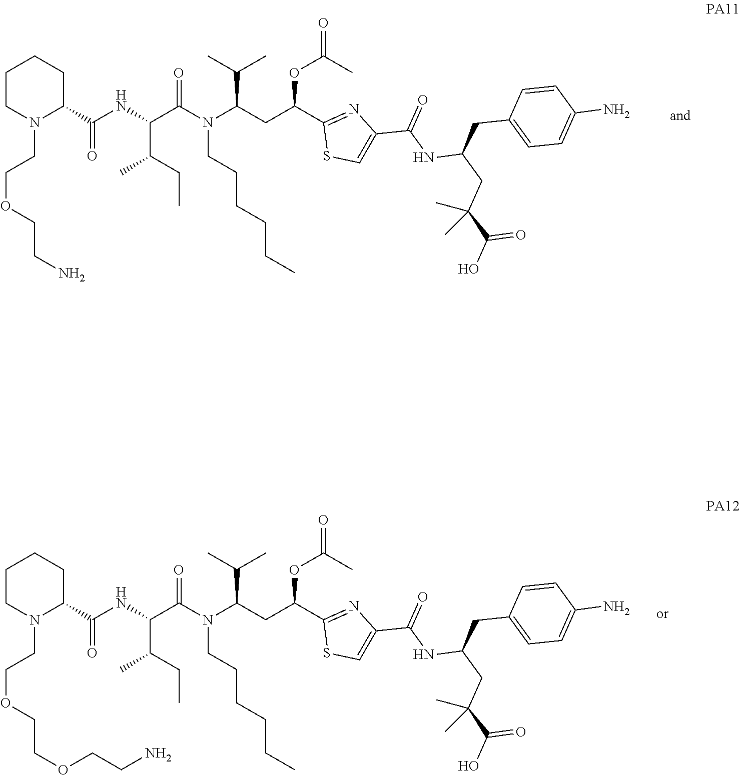

a pharmaceutically acceptable salt thereof.

In certain embodiments, set forth herein is a compound having the structure of Formula IV

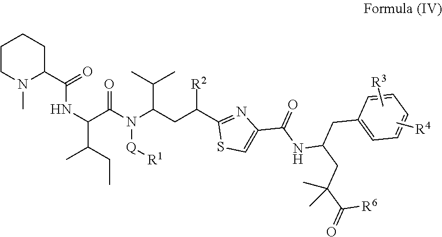

or a pharmaceutically acceptable salt thereof. In certain embodiments, R¹, R₂, R³, R⁴, and R⁶ are as described in the context of Formula I, above. In certain embodiments, R² is —O—C(O)CH₃, —O—C(O)—NH—(CH₂)₂—OH, —O—C(O)—NH—CH₂—CH(OH)—CH₂OH, —O—C(O)—NH—(CH₂CH₂O)₂—(CH₂)₂OH, or —NH—CH₂—C(O)—NH—(CH₂CH₂O)₂—(CH₂)₂NH. In certain embodiments, R² is —O—C(O)CH₃. In certain embodiments, R² is —O—C(O)—NH—(CH₂)₂—OH. In certain embodiments, R² is —O—C(O)—NH—CH₂—CH(OH)—CH₂OH. In certain embodiments, R² is —O—C(O)—NH—(CH₂CH₂O)₂—(CH₂)₂OH. In certain embodiments, R² is —NH—CH₂—C(O)—NH—(CH₂CH₂O)₂—(CH₂)₂NH. In certain embodiments, R⁶ is —OH.

In certain embodiments, provided herein are compounds according to Formula IV, selected from the group consisting of

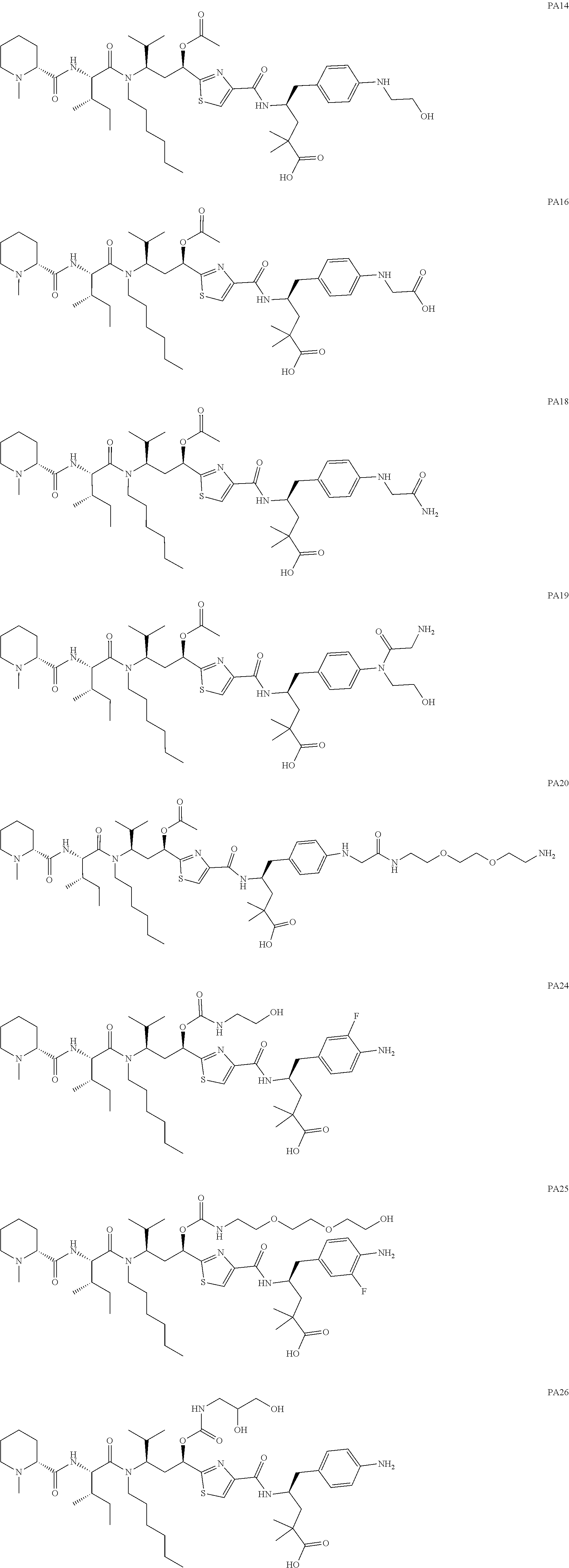

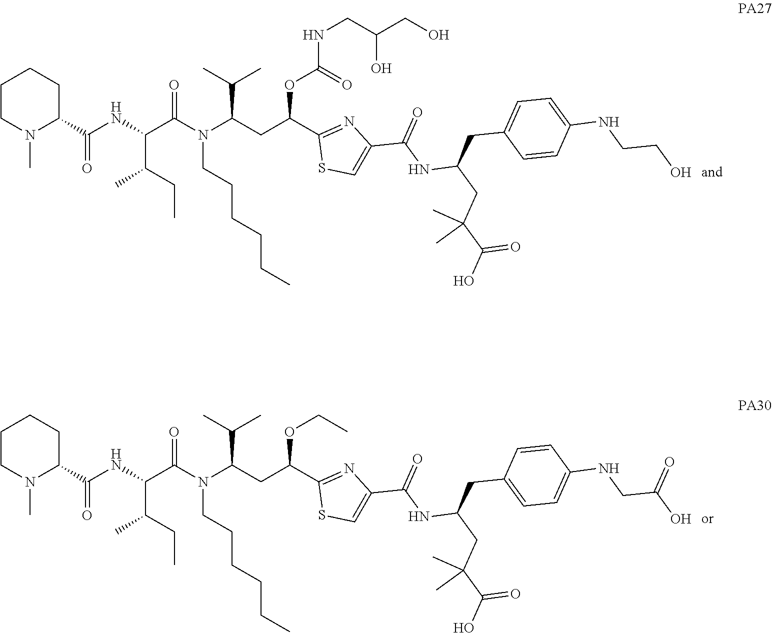 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(NCCO)cc2)CC(C)(C)C(=O)O)cs1)C(C)C ; CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCC(=O)O)cc2)CC(C)(C)C(=O)O)cs1)C(C)C]

a pharmaceutically acceptable salt thereof.

In Formula IV, in certain embodiments, R² is —O—C(O)CH₃ and R⁶ is —NH—C(O)OH. In certain embodiments, R² is —O—C(O)CH₃. In certain embodiments, R⁶ is —NH—C(O)OH.

In certain embodiments, provided herein is compound according to Formula IV having the following structure

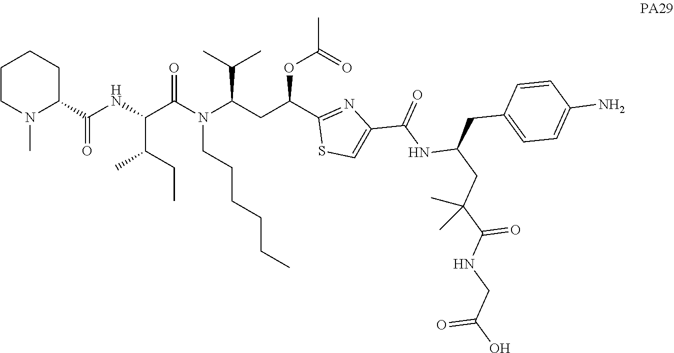 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)NCC(=O)O)cs1)C(C)C]

a pharmaceutically acceptable salt thereof.

In Formula IV, in certain embodiments, R² is —O—CH₂CH₃ or —O—(CH₂)₃—OH, and R⁶ is —OH. In certain embodiments, R² is —O—CH₂CH₃. In certain embodiments, R² is —O—(CH₂)₃—OH. In certain embodiments, R⁶ is —OH.

In certain embodiments, provided herein are compounds according to Formula IV, selected from the group consisting of

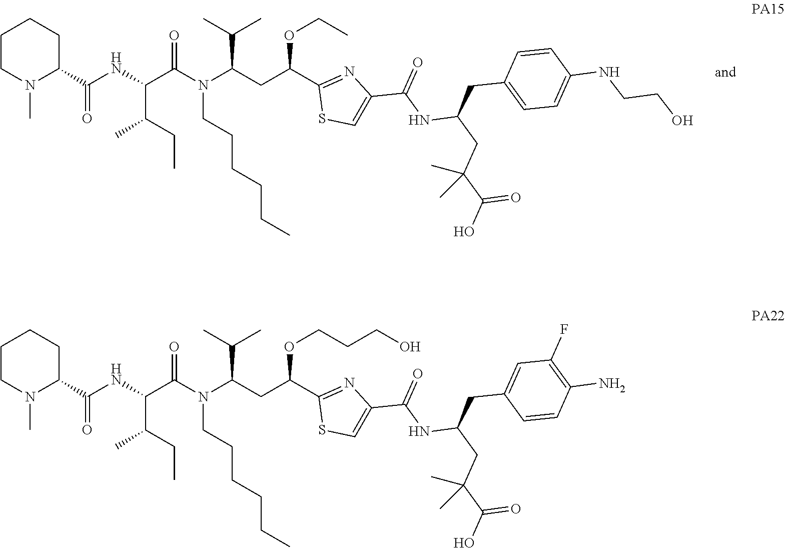 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCO)cc2)CC(C)(C)C(=O)O)cs1)C(C)C ; CCCCCCN(C(=O)[C@@H](NC(=O)[C@H]1CCCCN1C)[C@@H](C)CC)[C@H](C[C@@H](OCCCO)c1nc(C(=O)N[C@@H](Cc2ccc(N)c(F)c2)CC(C)(C)C(=O)O)cs1)C(C)C]

or
a pharmaceutically acceptable salt thereof.

In Formula IV, in certain embodiments, R² is —N—C(O)CH₃ and R⁶ is —OH. In certain embodiments, R² is —N—C(O)CH₃. In certain embodiments, R⁶ is —OH.

In certain embodiments, provided herein is compound according to Formula IV having the following structure

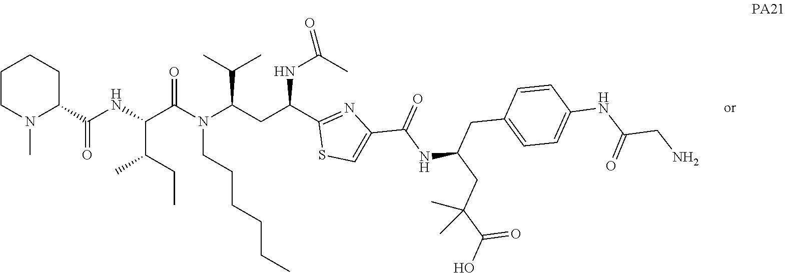 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(NC(=O)CN)cc2)CC(C)(C)C(=O)O)cs1)C(C)C]

a pharmaceutically acceptable salt thereof.

In certain embodiments, set forth herein is a compound having the structure of Formula V

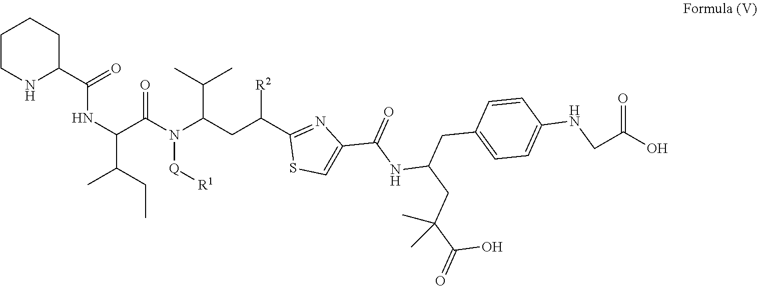

or a pharmaceutically acceptable salt thereof.

In Formula V, in certain embodiments, R² is —O—C(O)CH₃ or —O—(CH₂)₃—OH. In certain embodiments, R² is —O—C(O)CH₃. In certain embodiments, R² is —O—(CH₂)₃—OH.

In certain embodiments, provided herein are compounds according to Formula V, selected from the group consisting of

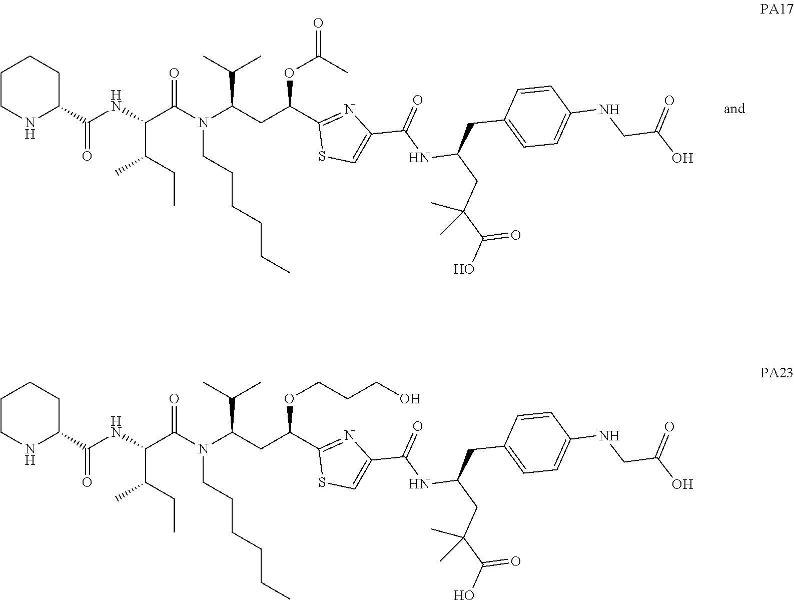 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C ; CCCCCCN(C(=O)[C@@H](NC(=O)[C@H]1CCCCN1)[C@@H](C)CC)[C@H](C[C@@H](OCCCO)c1nc(C(=O)N[C@@H](Cc2ccc(NCC(=O)O)cc2)CC(C)(C)C(=O)O)cs1)C(C)C]

or
a pharmaceutically acceptable salt thereof.

In certain embodiments, set forth herein is a compound having the structure of Formula VI

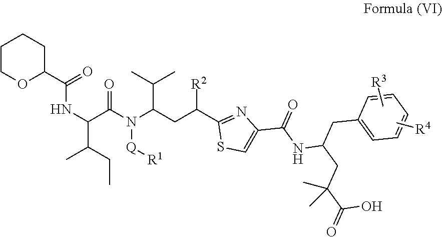

or a pharmaceutically acceptable salt thereof.

In certain embodiments, provided herein is compound according to Formula VI having the following structure

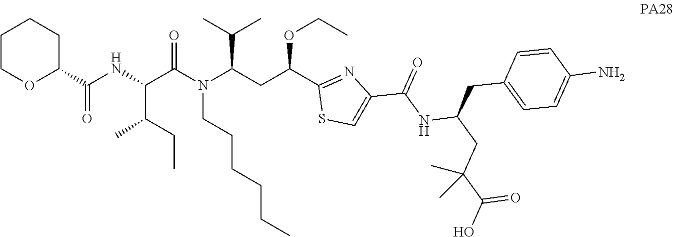 [SMILES: CCCCCCN(C(=O)[C@@H](NC(=O)[C@H]1CCCCO1)[C@@H](C)CC)[C@H](C[C@@H](OCC)c1nc(C(=O)N[C@@H](Cc2ccc(N)cc2)CC(C)(C)C(=O)O)cs1)C(C)C]

or
a pharmaceutically acceptable salt thereof.

In certain embodiments, T is not a compound selected from the following table

| ID. | Structure |
|---|---|
| P1 | 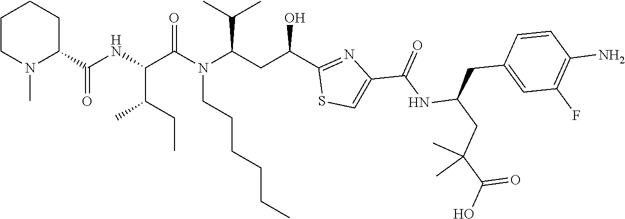 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 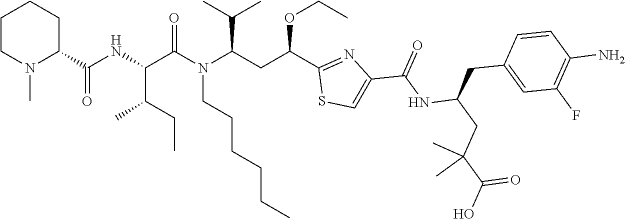 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 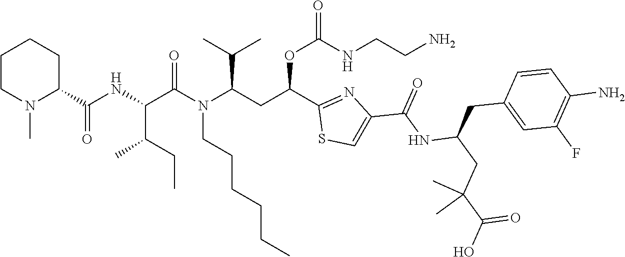 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 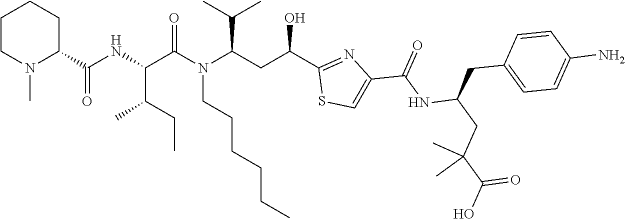 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 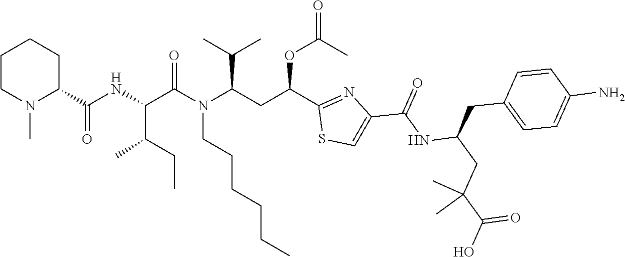 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 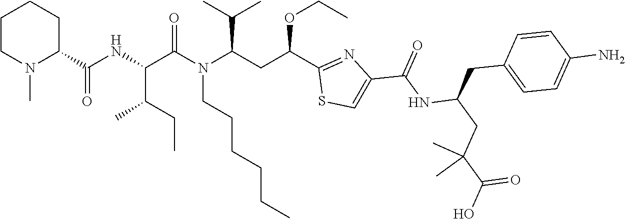 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 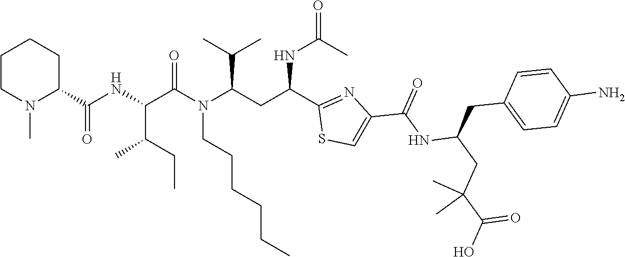 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 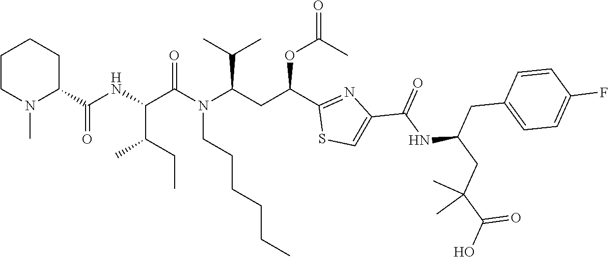 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 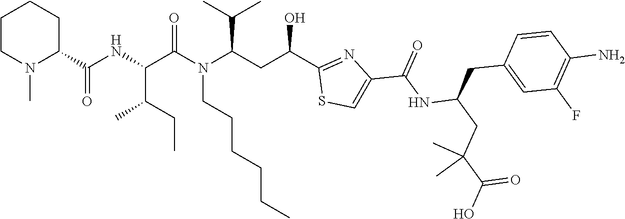 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 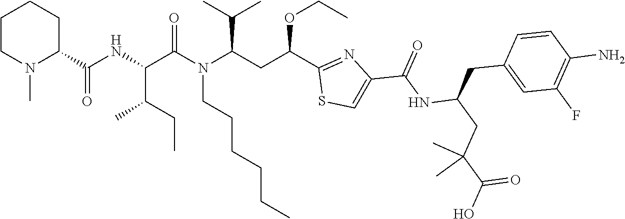 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 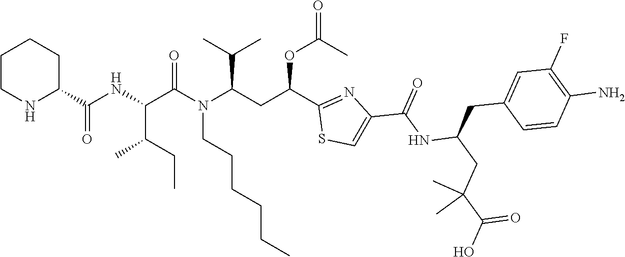 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 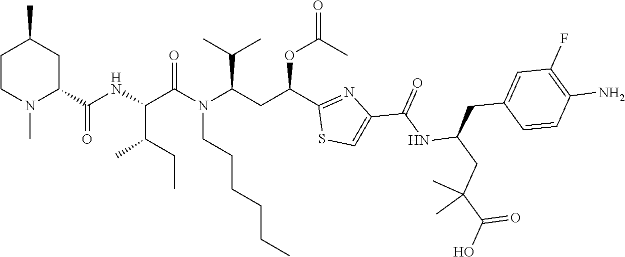 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 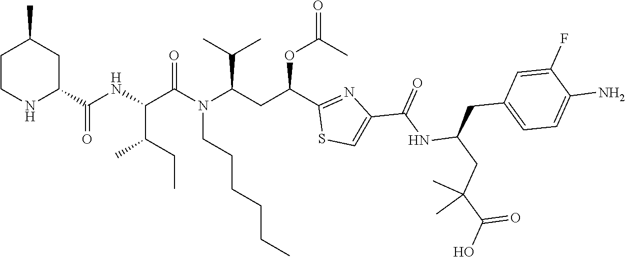 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 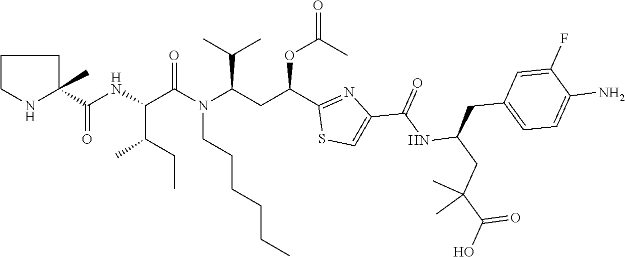 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 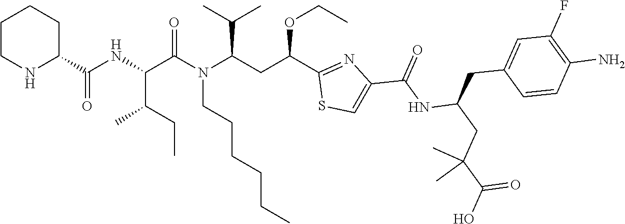 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 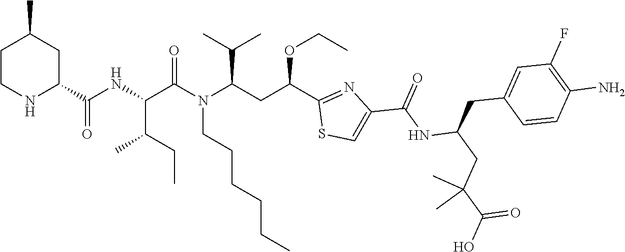 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 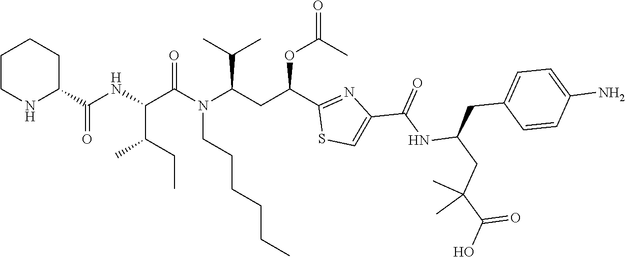 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 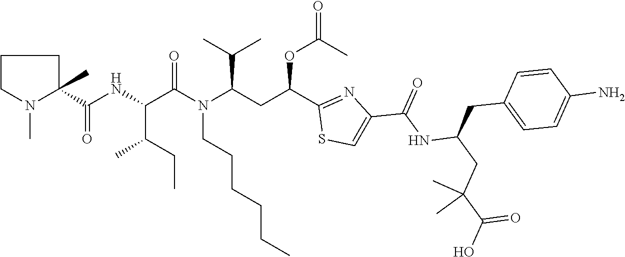 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 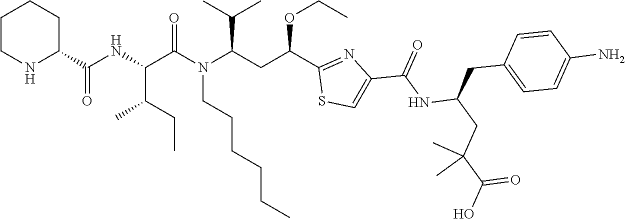 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 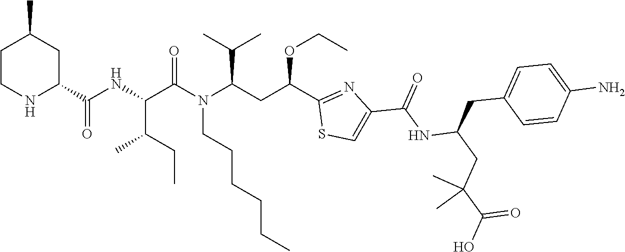 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 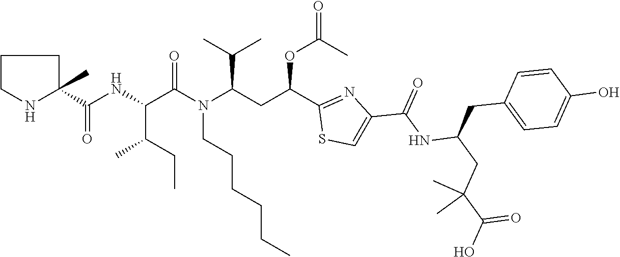 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 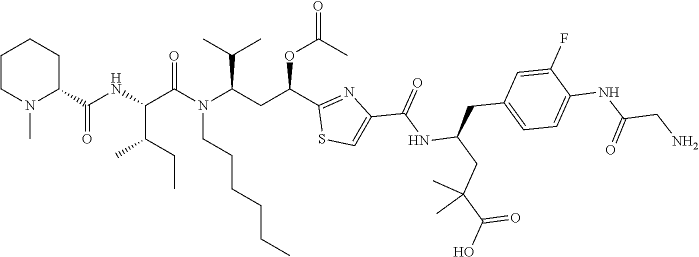 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 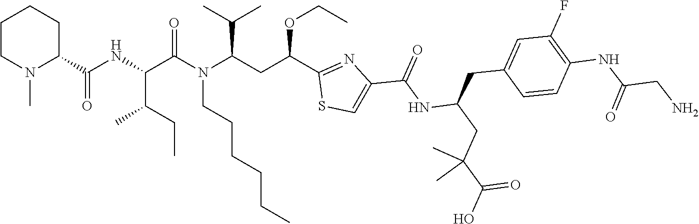 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 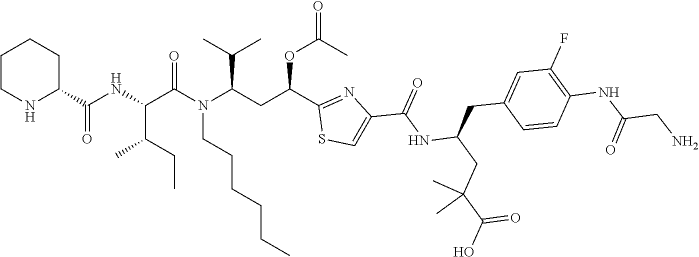 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 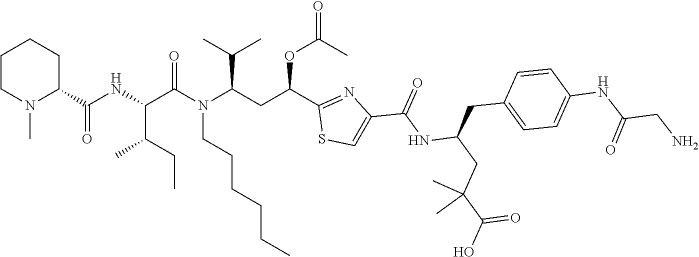 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 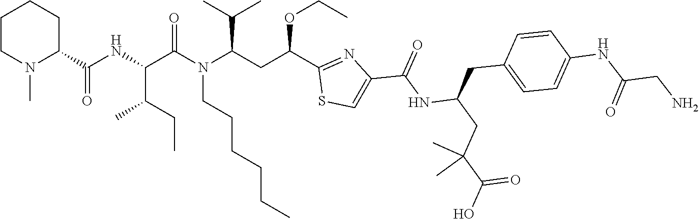 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 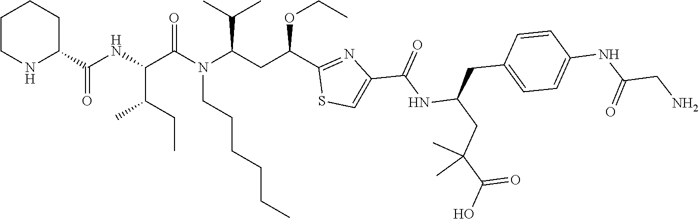 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 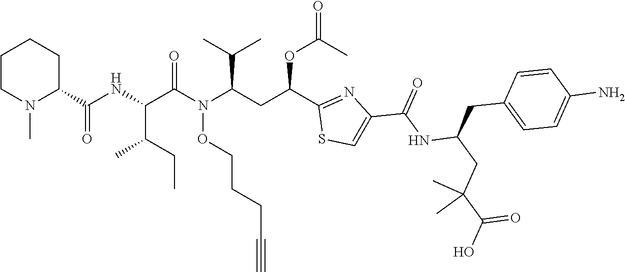 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 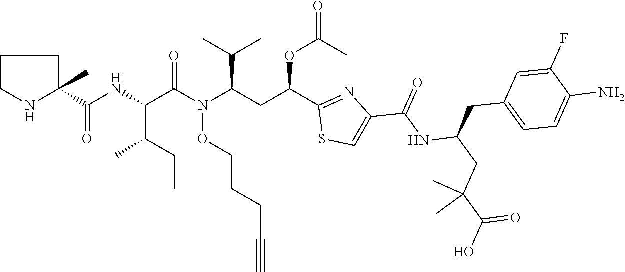 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 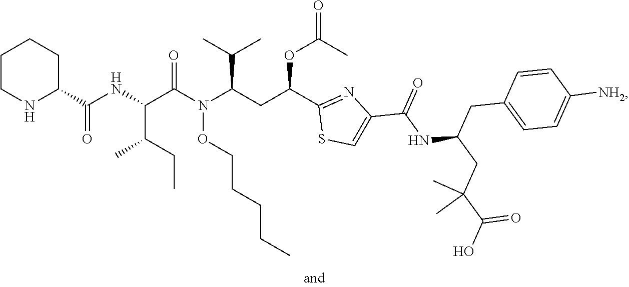 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 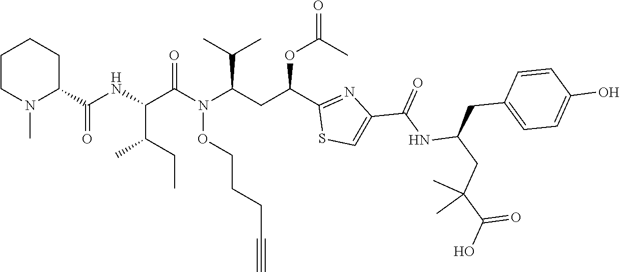 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 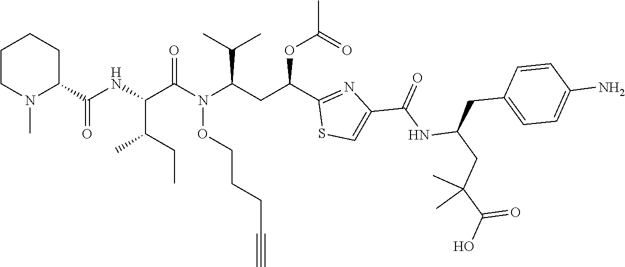 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 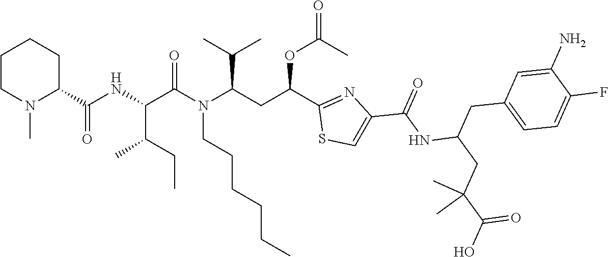 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 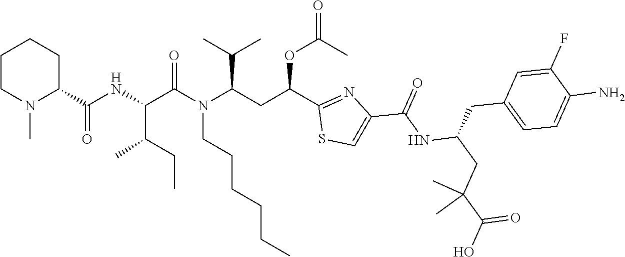 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 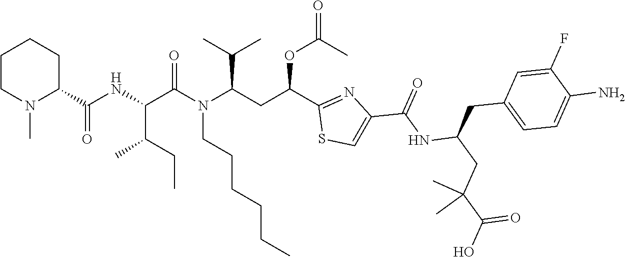 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 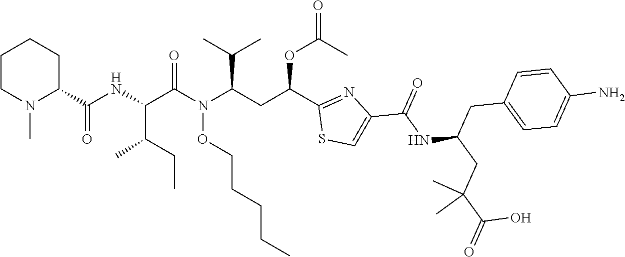 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 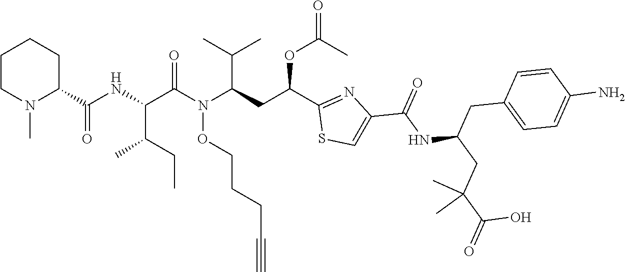 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 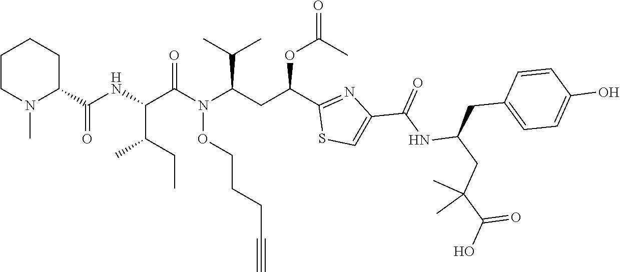 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 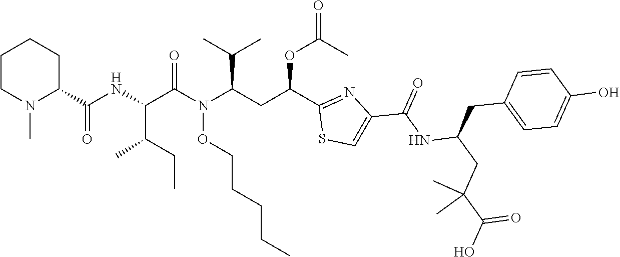 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 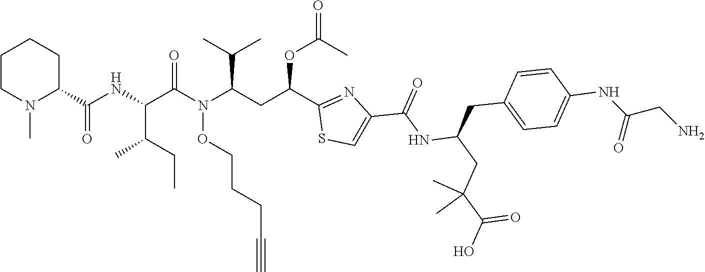 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

In certain embodiments, T is not a compound selected from the table in this paragraph or a residue thereof. In certain embodiments, T is not a compound selected from the table in this paragraph and bound to L. In certain embodiments, T is not a compound selected from the table in this paragraph or a salt thereof. In certain embodiments, T is not a compound selected from the table in this paragraph or a pharmaceutically acceptable salt thereof. In certain embodiments, T is a compound selected from the table in this paragraph or a polymorphic form thereof as measured by X-ray powder diffraction (XRPD), thermogravimetric analysis (TGA), and/or differential scanning calorimetry (DSC).

### Binding Agents

Suitable binding agents for any of the conjugates provided in the instant disclosure include, but are not limited to, antibodies, lymphokines (e.g., IL-2 or IL-3), hormones (e.g., insulin and glucocorticoids), growth factors (e.g., EGF, transferrin, and fibronectin type III), viral receptors, interleukins, or any other cell binding or peptide binding molecules or substances. Binding agents also include, but are not limited to, ankyrin repeat proteins and interferons.

In some embodiments, the binding agent is an antibody or an antigen-binding fragment thereof. The antibody can be in any form known to those of skill in the art. The term “antibody,” as used herein, refers to any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains interconnected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, CH₁, CH₂, and CH₃. Each light chain comprises a light chain variable region (abbreviated herein as LCVR or V_{L}) and a light chain constant region. The light chain constant region comprises one domain, C_{L}1. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, and FR4. In different embodiments disclosed herein, the FRs of the antibodies (or antigen-binding portion or fragment thereof) suitable for the compounds described herein may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable, standard technique(s) such as proteolytic digestion or recombinant genetic engineering technique(s) involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L}, or V_{L}-V_{L} dimers. Alternatively, the antigen-binding fragment of an antibody may contain a monomeric V_{H} or V_{L} domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of this disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-CH₂—C_{H}3; V_{H}-CL; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least two (e.g., five, ten, fifteen, twenty, forty, sixty, or more) amino acids which result in a flexible or semi-flexible linkage between adjacent variable and/or constant domains in a single polypeptide molecule. As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of this disclosure using routine techniques available in the art. In certain embodiments described herein, antibodies described herein are human antibodies. The term “human antibody,” as used herein, is intended to include antibodies having variable and constant regions derived from human germline immunoglobulin sequences. The human antibodies of this disclosure may include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody,” as used herein, is not intended to include antibodies in which CDR sequences derived from the germline of another mammalian species, such as a mouse, have been grafted onto human framework sequences. The term “human antibody” does not include naturally occurring molecules that normally exist without modification or human intervention/manipulation, in a naturally occurring, unmodified living organism. The antibodies disclosed herein may, in some embodiments, be recombinant human antibodies. The term “recombinant human antibody,” as used herein, is intended to include all human antibodies that are prepared, expressed, created, or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created, or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via interchain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2, or C_{H}3 region which may be desirable, for example, in production, to improve the yield of the desired antibody form. The antibodies described herein may be isolated antibodies. An “isolated antibody,” as used herein, refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the instant disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals. The antibodies used herein can comprise one or more amino acid substitutions, insertions, and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. This disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, for example, only the mutated residues found within the first eight amino acids of FR1 or within the last eight amino acids of FR4, or only the mutated residues found within CDR1, CDR2, or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of this disclosure may contain any combination of two or more germline mutations within the framework and/or CDR regions, for example, wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within this disclosure. Antibodies useful for the compounds herein also include antibodies comprising variants of any of the HCVR, LCVR, and/or CDR amino acid sequences disclosed herein having one or more conservative substitutions. The term “epitope” refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

In certain embodiments, the antibody comprises a light chain. In certain embodiments, the light chain is a kappa light chain. In certain embodiments, the light chain is a lambda light chain. In certain embodiments, the antibody comprises a heavy chain. In some embodiments, the heavy chain is an IgA. In some embodiments, the heavy chain is an IgD. In some embodiments, the heavy chain is an IgE. In some embodiments, the heavy chain is an IgG. In some embodiments, the heavy chain is an IgM. In some embodiments, the heavy chain is an IgG1. In some embodiments, the heavy chain is an IgG2. In some embodiments, the heavy chain is an IgG3. In some embodiments, the heavy chain is an IgG4. In some embodiments, the heavy chain is an IgA1. In some embodiments, the heavy chain is an IgA2.

In some embodiments, the antibody is an antibody fragment. In some embodiments, the antibody fragment is an Fv fragment. In some embodiments, the antibody fragment is a Fab fragment. In some embodiments, the antibody fragment is a F(ab′)₂ fragment. In some embodiments, the antibody fragment is a Fab′ fragment. In some embodiments, the antibody fragment is an scFv (sFv) fragment. In some embodiments, the antibody fragment is an scFv-Fc fragment.

In some embodiments, the antibody is a monoclonal antibody. In some embodiments, the antibody is a polyclonal antibody. In some embodiments, the antibody is a bispecific antibody including a first antigen-binding domain (also referred to herein as “D1”), and a second antigen-binding domain (also referred to herein as “D2”).

As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., PRLR or STEAP2). The term “specifically binds” or the like, as used herein, means that the antigen-binding domain forms a complex with a particular antigen characterized by a dissociation constant (KD) of 1 μM or less, and does not bind other unrelated antigens under ordinary test conditions. “Unrelated antigens” are proteins, peptides, or polypeptides that have less than 95% amino acid identity to one another.

Exemplary categories of antigen-binding domains that can be used in the context of this disclosure include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. For example, an antigen-binding domain, as used in the context of this disclosure, includes polypeptides that bind a particular antigen (e.g., a target molecule [T] or an internalizing effector protein [E]) or a portion thereof with a K_{D} of less than about 1 less than about 500 nM, less than about 250 nM, less than about 125 nM, less than about 60 nM, less than about 30 nM, less than about 10 nM, less than about 5 nM, less than about 2 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

In some embodiments, the antibody is a chimeric antibody. In some embodiments, the antibody is a humanized antibody. In some embodiments, the antibody is a human antibody.

In some embodiments, the antibody is an anti-PSMA, anti-PRLR, anti-MUC16, anti-HER2, anti-EGFRvIII, anti-MET, or anti-STEAP2 antibody or antigen binding fragments thereof. In some embodiments, the antibody or antigen-binding fragment is anti-PSMA. In some embodiments, the antibody or antigen-binding fragment is anti-MUC16. In some embodiments, the antibody or antigen-binding fragment is anti-HER2. In some embodiments, the antibody or antigen-binding fragment is anti-EGFRvIII. In some embodiments, the antibody or antigen-binding fragment is anti-MET. In some embodiments, the antibody or antigen-binding fragment is anti-PRLR or anti-STEAP2. In some embodiments, the antibody is an anti-PRLR or anti-HER2 antibody. In some embodiments, the antibody or antigen-binding fragment thereof is anti-STEAP2. In some embodiments, the antibody or antigen-binding fragment thereof is anti-PRLR.

The antibody can have binding specificity for any antigen deemed suitable to those of skill in the art. In certain embodiments, the antigen is a transmembrane molecule (e.g., receptor). In one embodiment, the antigen is expressed on a tumor. In some embodiments, the binding agents interact with or bind to tumor antigens, including antigens specific for a type of tumor or antigens that are shared, overexpressed, or modified on a particular type of tumor. In one embodiment, the antigen is expressed on solid tumors. Exemplary antigens include, but are not limited to, lipoproteins; alpha1-antitrypsin; a cytotoxic T-lymphocyte associated antigen (CTLA), such as CTLA-4 or CTLA4; vascular endothelial growth factor (VEGF); receptors for hormones or growth factors; protein A or D; fibroblast growth factor receptor 2 (FGFR2), EpCAM or Epcam, GD3, FLT3, PSMA, PSCA, MUC1, MUC16 or Muc16, STEAP, STEAP2, CEA, TENB2, EphA receptors, EphB receptors, folate receptor, FOLRI, mesothelin, cripto, alphavbeta6, integrins, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152, or an antibody which binds to one or more tumor-associated antigens or cell-surface receptors disclosed in U.S. Patent Application Publication No. 2008/0171040 or U.S. Patent Application Publication No. 2008/0305044 each incorporated herein in their entirety by reference; erythropoietin; osteoinductive factors; immunotoxins; a bone morphogenetic protein (BMP); T-cell receptors; surface membrane proteins; integrins, such as CD11a, CD11b, CD11c, CD18, an ICAM, VLA-4 and VCAM; a tumor associated antigen such as AFP, ALK, B7H4, BAGE proteins, β-catenin, brc-abl, BRCA1, BORIS, CA9 (carbonic anhydrase IX), caspase-8, CD123, CDK4, CEA, CLEC12A, c-kit, cMET, cyclin-B1, CYP1B1, EGFRvIII, endoglin, EphA2, ErbB2/Her2, ErbB3/Her3, ErbB4/Her4, ETV6-AML, Fra-1, FOLR1, GAGE proteins, GD2, GloboH, glypican-3, GM3, gp100, Her2, HLA/B-raf, HLA/EBNA1, HLA/k-ras, HLA/MAGE-A3, hTERT, IGF1R, LGR5, LMP2, MAGE proteins, MART-1, mesothelin, ML-IAP, Muc1, CA-125, MUM1, NA17, NGEP, NY-BR1, NY-BR62, NY-BR85, NY-ESO1, OX40, p15, p53, PAP, PAX3, PAX5, PCTA-1, PDGFR-α, PDGFR-β, PDGF-A, PDGF-B, PDGF-C, PDGF-D, PLAC1, PRLR, PRAME, PSCA, PSGR, PSMA (FOLH1), RAGE proteins, Ras, RGS5, Rho, SART-1, SART-3, Steap-1, Steap-2, STn, survivin, TAG-72, TGF-β, TMPRSS2, Tn, TNFRSF17, TRP-1, TRP-2, tyrosinase, and uroplakin-3, and fragments of any of the above-listed polypeptides; cell-surface expressed antigens; c-MET; molecules such as class A scavenger receptors including scavenger receptor A (SR-A), and other membrane proteins such as B7 family-related member including V-set and Ig domain-containing 4 (VSIG4), Colony stimulating factor 1 receptor (CSF1R), asialoglycoprotein receptor (ASGPR), and Amyloid beta precursor-like protein 2 (APLP-2). In some embodiments, the antigen is PRLR or HER2. In some embodiments, the antigen is STEAP2. In some embodiments, the antigen is human STEAP2. In some examples, the MAGE proteins are selected from MAGE-1, -2, -3, -4, -6, and -12. In some examples, the GAGE proteins are selected from GAGE-1 and GAGE-2.

Exemplary antigens also include, but are not limited to, BCMA, SLAMF7, GPNMB, and UPK3A. Exemplary antigens also include, but are not limited to, MUC16, STEAP2, and HER2.

In some embodiments, the antigens include MUC16. In some embodiments, the antigens include STEAP2. In some embodiments, the antigens include PSMA. In some embodiments, the antigens include HER2. In some embodiments, the antigen is prolactin receptor (PRLR) or prostate-specific membrane antigen (PSMA). In some embodiments, the antigen is MUC16. In some embodiments, the antigen is PSMA. In some embodiments, the antigen is HER2. In some embodiments, the antigen is STEAP2.

In certain embodiments, the antibody comprises a glutamine residue at one or more heavy chain positions numbered 295 in the EU numbering system. In this disclosure, this position is referred to as glutamine 295, or as Gln295, or as Q295. Those of skill will recognize that this is a conserved glutamine residue in the wild-type sequence of many antibodies. In other useful embodiments, the antibody can be engineered to comprise a glutamine residue. In certain embodiments, the antibody comprises one or more N297Q mutations. Techniques for modifying an antibody sequence to include a glutamine residue are within the skill of those in the art (see, e.g., Ausubel et al. Current Protoc. Mol. Biol.).

In some embodiments, the antibody, or antigen-binding fragment thereof, conjugated to the linker-payload or payload can be an antibody that targets STEAP2. Suitable anti-STEAP2 antibodies or antigen binding fragments thereof include those, for example, in International Publication No. WO 2018/058001 A1, including those comprising amino acid sequences disclosed in Table 1, on page 75 therein. In some embodiments, an anti-STEAP2 antibody is H1H7814N of WO 2018/058001 A1, comprising the CDRs of H1M7814N in the same publication. In some embodiments, an anti-STEAP2 antibody comprises a heavy chain complementarity determining region (HCDR)-1 comprising SEQ ID NO: 2; an HCDR2 comprising SEQ ID NO: 3; an HCDR3 comprising SEQ ID NO: 4; a light chain complementarity determining region (LCDR)-1 comprising SEQ ID NO: 6; an LCDR2 comprising SEQ ID NO: 7; and an LCDR3 comprising SEQ ID NO: 8. In some embodiments, an anti-STEAP2 antibody comprises a heavy chain variable region (HCVR) comprising SEQ ID NO: 1 and a light chain variable region (LCVR) comprising SEQ ID NO: 5. In any of the foregoing embodiments, the anti-STEAP2 antibody can be prepared by site-directed mutagenesis to insert a glutamine residue at a site without resulting in disabled antibody function or binding. For example, in any of the foregoing embodiments, the anti-STEAP2 antibody can comprise an Asn297Gln (N297Q) mutation. Such antibodies having an N297Q mutation can also contain one or more additional naturally occurring glutamine residues in their variable regions, which can be accessible to transglutaminase and therefore capable of conjugation to a payload or a linker-payload (Table A). In certain embodiments, the antibody or antigen-binding fragment thereof comprises three heavy chain complementarity determining regions (HCDR1, HCDR2, and HCDR3) within a heavy chain variable region (HCVR) amino acid sequence of SEQ ID NO:1; and three light chain complementarity determining regions (LCDR1, LCDR2, and LCDR3) within a light chain variable region (LCVR) amino acid sequence of SEQ ID NO:5. In certain embodiments, the antibody or antigen-binding fragment thereof comprises an HCVR amino acid sequence of SEQ ID NO:1; and an LCVR amino acid sequence of SEQ ID NO:5. International Publication No. WO 2018/058001 A1 is hereby incorporated herein by reference in its entirety.

In some embodiments, the antibody, or antigen-binding fragment thereof, conjugated to the linker-payload or payload can be an antibody that targets human prolactin receptor (PRLR). Suitable anti-PRLR antibodies or antigen-binding fragments thereof include those, for example, in International Publication No. WO 2015/026907 A1, including those comprising amino acid sequences disclosed in Table 1, on page 36 therein. In some embodiments, an anti-PRLR antibody is H1H6958N2 of WO 2015/026907 A1, comprising the CDRs of H2M6958N2 in the same publication. In some embodiments, an anti-PRLR antibody comprises a heavy chain complementarity determining region (HCDR)-1 comprising SEQ ID NO: 10; an HCDR2 comprising SEQ ID NO: 11; an HCDR3 comprising SEQ ID NO: 12; a light chain complementarity determining region (LCDR)-1 comprising SEQ ID NO: 14; an LCDR2 comprising SEQ ID NO: 15; and an LCDR3 comprising SEQ ID NO: 16. In some embodiments, an anti-PRLR antibody comprises a heavy chain variable region (HCVR) comprising SEQ ID NO: 9 and a light chain variable region (LCVR) comprising SEQ ID NO: 13. In any of the foregoing embodiments, the anti-PRLR antibody can be prepared by site-directed mutagenesis to insert a glutamine residue at a site without resulting in disabled antibody function or binding. For example, in any of the foregoing embodiments, the anti-PRLR antibody can comprise an Asn297Gln (N297Q) mutation. Such antibodies having an N297Q mutation can also contain one or more additional naturally occurring glutamine residues in their variable regions, which can be accessible to transglutaminase and therefore capable of conjugation to a payload or a linker-payload (Table A). In certain embodiments, the antibody or antigen-binding fragment thereof comprises three heavy chain complementarity determining regions (HCDR1, HCDR2, and HCDR3) within a heavy chain variable region (HCVR) amino acid sequence of SEQ ID NO:9; and three light chain complementarity determining regions (LCDR1, LCDR2, and LCDR3) within a light chain variable region (LCVR) amino acid sequence of SEQ ID NO:13. In certain embodiments, the antibody or antigen-binding fragment thereof comprises an HCVR amino acid sequence of SEQ ID NO:9; and an LCVR amino acid sequence of SEQ ID NO:13. International Publication No. WO 2015/026907 A1 is hereby incorporated herein by reference in its entirety.

TABLE A
Sequences of Exemplary Antibodies H1H7814N
(anti-STEAP2) and H1H6958N2 (anti-PRLR)

| SEQ ID | Molecule/ | | |
|---|---|---|---|
| NO: | Antibody | Region | Sequence |
| 1 | H1H7814N | HCVR | QVQLVESGGGVVQPGRSLRLS |
| | | | CVASGFTISSYGMNWVRQAPG |
| | | | KGLEWVAVISYDGGNKYSVDS |
| | | | VKGRFTISRDNSKNTLYLQMN |
| | | | SLRAEDSAVYYCARGRYFDLW |
| | | | GRGTLVTVSS |
| 2 | H1H7814N | HCDR1 | GFTISSYG |
| 3 | H1H7814N | HCDR2 | ISYDGGNK |
| 4 | H1H7814N | HCDR3 | ARGRYFDL |
| 5 | H1H7814N | LCVR | DIQMTQSPSTLSASVGDRVTI |
| | | | TCRASQSISSWLAWYQQKPGR |
| | | | APNLLISKASSLKSGVPSRFS |
| | | | GSGSGTEFTLTVSSLQPDDFA |
| | | | TYYCQQYYSYSYTFGQGTKLE |
| | | | IK |
| 6 | H1H7814N | LCDR1 | QSISSW |
| 7 | H1H7814N | LCDR2 | KAS |
| 8 | H1H7814N | LCDR3 | QQYYSYSYT |
| 9 | H1H6958N2 | HCVR | QVQLVESGGGVVQPGRSLRLS |
| | | | CGASGFTFRNYGMQWVRQGPG |
| | | | KGLEWVTLISFDGNDKYYADS |
| | | | VKGRFTISRDNSKNTLFLQMN |
| | | | SLRTEDTAVYYCARGGDFDYW |
| | | | GQGTLVTVSS |
| 10 | H1H6958N2 | HCDR1 | GFTFRNYG |
| 11 | H1H6958N2 | HCDR2 | ISFDGNDK |
| 12 | H1H6958N2 | HCDR3 | ARGGDFDY |
| 13 | H1H6958N2 | LCVR | DIQMTQSPSSLSASVGDRVTI |
| | | | TCRASQDIRKDLGWYQQKPGK |
| | | | APKRLIYAASSLHSGVPSRES |
| | | | GSGSGTEFTLTISSLQPEDFA |
| | | | TYYCLQHNSYPMYTFGQGTKL |
| | | | EIK |
| 14 | H1H6958N2 | LCDR1 | QDIRKD |
| 15 | H1H6958N2 | LCDR2 | AAS |
| 16 | H1H6958N2 | LCDR3 | LQHNSYPMYT |
| 17 | hPRLR | | MHRPRRRGTRPPPLALLAALL |
| | ecto-MMH | | LAARGADAQLPPGKPEIFKCR |
| | | | SPNKETFTCWWRPGTDGGLPT |
| | | | NYSLTYHREGETLMHECPDYI |
| | | | TGGPNSCHFGKQYTSMWRTYI |
| | | | MMVNATNQMGSSFSDELYVDV |
| | | | TYIVQPDPPLELAVEVKQPED |
| | | | RKPYLWIKWSPPTLIDLKTGW |
| | | | FTLLYEIRLKPEKAAEWEIHF |
| | | | AGQQTEFKILSLHPGQKYLVQ |
| | | | VRCKPDHGYWSAWSPATFIQI |
| | | | PSDFTMNDEQKLISEEDLGGE |
| | | | QKLISEEDLHHHHHH |

This disclosure provides antibodies or antigen-binding fragments thereof that specifically bind STEAP2, comprising an HCVR comprising an amino acid sequence selected from any of the HCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind STEAP2, comprising an LCVR comprising an amino acid sequence selected from any of the LCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind STEAP2, comprising an HCVR and an LCVR amino acid sequence pair (HCVR/LCVR) comprising any of the HCVR amino acid sequences listed in Table A paired with any of the LCVR amino acid sequences listed in Table A. According to certain embodiments, this disclosure provides antibodies, or antigen-binding fragments thereof, comprising an HCVR/LCVR amino acid sequence pair contained within any of the exemplary anti-STEAP2 antibodies listed in Table A. In certain embodiments, the HCVR/LCVR amino acid sequence pair is selected from the group consisting of 250/258; as described in International Publication No. WO 2018/058001 A1, the contents of which are incorporated herein by reference in its entirety.

This disclosure also provides antibodies or antigen-binding fragments thereof that specifically bind STEAP2, comprising a heavy chain CDR1 (HCDR1) comprising an amino acid sequence selected from any of the HCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a heavy chain CDR2 (HCDR2) comprising an amino acid sequence selected from any of the HCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a heavy chain CDR3 (HCDR3) comprising an amino acid sequence selected from any of the HCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a light chain CDR1 (LCDR1) comprising an amino acid sequence selected from any of the LCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a light chain CDR2 (LCDR2) comprising an amino acid sequence selected from any of the LCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising a light chain CDR3 (LCDR3) comprising an amino acid sequence selected from any of the LCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind STEAP2, comprising an HCDR3 and an LCDR3 amino acid sequence pair (HCDR3/LCDR3) comprising any of the HCDR3 amino acid sequences listed in Table A paired with any of the LCDR3 amino acid sequences listed in Table A. According to certain embodiments, this disclosure provides antibodies, or antigen-binding fragments thereof, comprising an HCDR3/LCDR3 amino acid sequence pair contained within any of the exemplary anti-STEAP2 antibodies listed in Table A. In certain embodiments, the HCDR3/LCDR3 amino acid sequence pair is selected from the group consisting of 256/254; as described in International Publication No. WO 2018/058001 A1, the contents of which are incorporated herein by reference in its entirety.

This disclosure also provides antibodies or antigen-binding fragments thereof that specifically bind STEAP2, comprising a set of six CDRs (i.e., HCDR1-HCDR2-HCDR3-LCDR1-LCDR2-LCDR3) contained within any of the exemplary anti-STEAP2 antibodies listed in Table A. In certain embodiments, the HCDR1-HCDR2-HCDR3-LCDR1-LCDR2-LCDR3 amino acid sequence set is selected from the group consisting of 252-254-256-260-262-264; as described in International Publication No. WO 2018/058001 A1, the contents of which are incorporated herein by reference in its entirety.

In a related embodiment, this disclosure provides antibodies, or antigen-binding fragments thereof that specifically bind STEAP2, comprising a set of six CDRs (i.e., HCDR1-HCDR2-HCDR3-LCDR1-LCDR2-LCDR3) contained within an HCVR/LCVR amino acid sequence pair as defined by any of the exemplary anti-STEAP2 antibodies listed in Table A. For example, this disclosure includes antibodies or antigen-binding fragments thereof that specifically bind STEAP2, comprising the HCDR1-HCDR2-HCDR3-LCDR1-LCDR2-LCDR3 amino acid sequence set contained within an HCVR/LCVR amino acid sequence pair selected from the group consisting of 250/258; as described in International Publication No. WO 2018/058001 A1, the contents of which are incorporated herein by reference in its entirety. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, for example, the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, for example, Kabat, “Sequences of Proteins of Immunological Interest,” National Institutes of Health, Bethesda, Md. (1991); A1-Lazikani et al. J. Mol. Biol. 273:927-948 (1997); and Martin et al. Proc. Natl. Acad. Sci. USA 86:9268-9272 (1989). Public databases are also available for identifying CDR sequences within an antibody.

This disclosure provides antibodies or antigen-binding fragments thereof that specifically bind PRLR, comprising an HCVR comprising an amino acid sequence selected from any of the HCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind PRLR, comprising an LCVR comprising an amino acid sequence selected from any of the LCVR amino acid sequences listed in Table A, or a substantially similar sequence thereof having at least 90%, at least 95%, at least 98%, or at least 99% sequence identity thereto.

This disclosure also provides antibodies or antigen-binding fragments thereof that specifically bind PRLR, comprising an HCVR and an LCVR amino acid sequence pair (HCVR/LCVR) comprising any of the HCVR amino acid sequences listed in Table A paired with any of the LCVR amino acid sequences listed in Table A. According to certain embodiments, this disclosure provides antibodies, or antigen-binding fragments thereof, comprising an HCVR/LCVR amino acid sequence pair contained within any of the exemplary anti-PRLR antibodies listed in Table A. In certain embodiments, the HCVR/LCVR amino acid sequence pair is selected from the group consisting of 18/26; 66/74; 274/282; 290/298; and 370/378; as described in International Publication No. WO 2015/026907 A1, the contents of which are incorporated herein by reference in its entirety.

This disclosure also provides antibodies or antigen-binding fragments thereof that specifically bind PRLR, comprising a heavy chain CDR1 (HCDR1) comprising an amino acid sequence selected from any of the HCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a heavy chain CDR2 (HCDR2) comprising an amino acid sequence selected from any of the HCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a heavy chain CDR3 (HCDR3) comprising an amino acid sequence selected from any of the HCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a light chain CDR1 (LCDR1) comprising an amino acid sequence selected from any of the LCDR1 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a light chain CDR2 (LCDR2) comprising an amino acid sequence selected from any of the LCDR2 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising a light chain CDR3 (LCDR3) comprising an amino acid sequence selected from any of the LCDR3 amino acid sequences listed in Table A or a substantially similar sequence thereof having at least 90%, at least 95%, at least 98%, or at least 99% sequence identity.

This disclosure also provides antibodies or antigen-binding fragments thereof that specifically bind PRLR, comprising an HCDR3 and an LCDR3 amino acid sequence pair (HCDR3/LCDR3) comprising any of the HCDR3 amino acid sequences listed in Table A paired with any of the LCDR3 amino acid sequences listed in Table A. According to certain embodiments, this disclosure provides antibodies, or antigen-binding fragments thereof, comprising an HCDR3/LCDR3 amino acid sequence pair contained within any of the exemplary anti-PRLR antibodies listed in Table A. In certain embodiments, the HCDR3/LCDR3 amino acid sequence pair is selected from the group consisting of 24/32; 72/80; 280/288; 296/304; and 376/384; as described in International Publication No. WO 2015/026907 A1, the contents of which are incorporated herein by reference in its entirety.

This disclosure also provides antibodies or antigen-binding fragments thereof that specifically bind PRLR, comprising a set of six CDRs (i.e., HCDR1-HCDR2-HCDR3-LCDR1-LCDR2-LCDR3) contained within any of the exemplary anti-PRLR antibodies listed in Table A. In certain embodiments, the HCDR1-HCDR2-HCDR3-LCDR1-LCDR2-LCDR3 amino acid sequence set is selected from the group consisting of 20-22-24-28-30-32; 68-70-72-76-78-80; 276-278-280-284-286-288; 292-294-296-300-302-304; and 372-374-376-380-382-384; as described in International Publication No. WO 2015/026907 A1, the contents of which are incorporated herein by reference in its entirety.

In a related embodiment, this disclosure provides antibodies, or antigen-binding fragments thereof that specifically bind PRLR, comprising a set of six CDRs (i.e., HCDR1-HCDR2-HCDR3-LCDR1-LCDR2-LCDR3) contained within an HCVR/LCVR amino acid sequence pair as defined by any of the exemplary anti-PRLR antibodies listed in Table A. For example, this disclosure includes antibodies or antigen-binding fragments thereof that specifically bind PRLR, comprising the HCDR1-HCDR2-HCDR3-LCDR1-LCDR2-LCDR3 amino acid sequence set contained within an HCVR/LCVR amino acid sequence pair selected from the group consisting of 18/26; 66/74; 274/282; 290/298; and 370/378; as described in International Publication No. WO 2015/026907 A1, the contents of which are incorporated herein by reference in its entirety. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, for example, the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, for example, Kabat, “Sequences of Proteins of Immunological Interest,” National Institutes of Health, Bethesda, Md. (1991); A1-Lazikani et al. J. Mol. Biol. 273:927-948 (1997); and Martin et al. Proc. Natl. Acad. Sci. USA 86:9268-9272 (1989). Public databases are also available for identifying CDR sequences within an antibody.

The binding agent linkers can be bonded to the binding agent, for example, antibody or antigen-binding molecule or fragment, through an attachment at a particular amino acid within the antibody or antigen-binding molecule or fragment. Exemplary amino acid attachments that can be used in the context of this embodiment of the disclosure include, for example, lysine (see, e.g., U.S. Pat. No. 5,208,020; U.S. 2010/0129314; Hollander et al. Bioconjugate Chem., 2008, 19:358-361; WO 2005/089808; U.S. Pat. No. 5,714,586; U.S. 2013/0101546; and U.S. 2012/0585592), cysteine (see, e.g., U.S. 2007/0258987; WO 2013/055993; WO 2013/055990; WO 2013/053873; WO 2013/053872; WO 2011/130598; U.S. 2013/0101546; and U.S. Pat. No. 7,750,116), selenocysteine (see, e.g., WO 2008/122039; and Hofer et al. Proc. Natl. Acad. Sci., USA, 2008, 105:12451-12456), formyl glycine (see, e.g., Carrico et al. Nat. Chem. Biol., 2007, 3:321-322; Agarwal et al. Proc. Natl. Acad. Sci., USA, 2013, 110:46-51, and Rabuka et al. Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., U.S. 2008/0305497, WO 2014/065661, and Ryan et al. Food & Agriculture Immunol., 2001, 13:127-130).

In some examples, the binding agent is an antibody or antigen binding molecule or fragment, and the antibody is bonded to the linker through a lysine residue. In some embodiments, the antibody or antigen binding molecule or fragment is bonded to the linker through a cysteine residue.

Linkers can also be conjugated to one or more glutamine residues via transglutaminase-based chemo-enzymatic conjugation (see, e.g., Dennler et al. Bioconjugate Chem. 2014, 25, 569-578). For example, in the presence of transglutaminase, one or more glutamine residues of an antibody can be coupled to a primary amine compound. Primary amine compounds include, for example, payloads or linker-payloads, which directly provide transglutaminase-modified antibody drug conjugates via transglutaminase-mediated coupling. Primary amine compounds also include linkers and spacers that are functionalized with reactive groups that can be subsequently treated with further compounds towards the synthesis of antibody-drug conjugates (e.g., in certain embodiments, transglutaminase-modified antibody drug conjugates). Antibodies comprising glutamine residues can be isolated from natural sources or engineered to comprise one or more glutamine residues. Techniques for engineering glutamine residues into an antibody polypeptide chain (glutaminyl-modified antibodies or antigen binding molecules) are within the skill of practitioners in the art. In certain embodiments, the antibody is aglycosylated.

In certain embodiments, the antibody, glutaminyl-modified antibody, or transglutaminase-modified antibody or antigen binding fragments thereof comprise at least one glutamine residue in at least one polypeptide chain sequence. In certain embodiments, the antibody, glutaminyl-modified antibody, or transglutaminase-modified antibody or antigen binding fragments thereof comprise two heavy chain polypeptides, each with one Gln295 or Q295 residue. In further embodiments, the antibody, glutaminyl-modified antibody, or transglutaminase-modified antibody or antigen binding fragments thereof comprise one or more glutamine residues at a site other than a heavy chain 295. Included herein are antibodies of this section bearing N297Q mutation(s) described herein.

### Primary Amine Compounds

In certain embodiments, primary amine compounds useful for the transglutaminase-mediated coupling of an antibody (or antigen binding compound or fragment) comprising one or more glutamine residues (i.e., resulting in a transglutaminase-modified antibody or antigen binding fragment thereof) can be any primary amine compound deemed useful by the practitioner of ordinary skill. Generally, the primary amine compound has the formula H₂N—R, where R can be any group compatible with the antibody and reaction conditions. In certain embodiments, R is alkyl, substituted alkyl, heteroalkyl, or substituted heteroalkyl.

In some embodiments, the primary amine compound comprises a reactive group or protected reactive group. Useful reactive groups include azides, alkynes, cycloalkynes, thiols, alcohols, ketones, aldehydes, carboxylic acids, esters, amides, hydrazides, anilines, and amines. In certain embodiments, the reactive group is selected from the group consisting of azide, alkyne, sulfhydryl, cycloalkyne, aldehyde, and carboxyl.

In certain embodiments, the primary amine compound is according to the formula H2N-LL-X, where LL is a divalent spacer and X is a reactive group or protected reactive group. In particular embodiments, LL is a divalent polyethylene glycol (PEG) group. In certain embodiments, X is selected from the group consisting of —SH, —N3, alkyne, aldehyde, and tetrazole. In particular embodiments, X is —N3.

In certain embodiments, the primary amine compound is according to one of the following formulae

H₂N—(CH₂)ₙ—X;

H₂N—(CH₂CH₂O)ₙ—(CH₂)ₚ—X;

H₂N—(CH₂)ₙ—N(H)C(O)—(CH₂)ₘ—X;

H₂N—(CH₂CH₂O)ₙ—N(H)C(O)—(CH₂CH₂O)ₘ—(CH₂)ₚ—X;

H₂N—(CH₂)ₙ—C(O)N(H)—(CH₂)ₘ—X;

H₂N—(CH₂CH₂O)ₙ—C(O)N(H)—(CH₂CH₂O)ₘ—(CH₂)ₚ—X;

H₂N—(CH₂)ₙ—N(H)C(O)—(CH₂CH₂O)ₘ—(CH₂)ₚ—X;

H₂N—(CH₂CH₂O)ₙ—N(H)C(O)—(CH₂)ₘ—X;

H₂N—(CH₂)ₙ—C(O)N(H)—(CH₂CH₂O)ₘ—(CH₂)ₚ—X; and

H₂N—(CH₂CH₂O)ₙ—C(O)N(H)—(CH₂)ₘ—X;

where n is an integer selected from one to twelse; m is an integer selected from zero to twelve; p is an integer selected from zero to two; and X is selected from the group consisting of —SH, —N₃, —C≡CH, —C(O)H, tetrazole, and any of

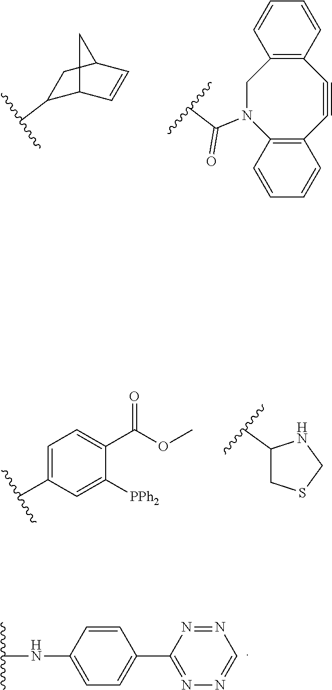

In the above embodiments, any of the alkyl or alkylene (i.e., —CH₂—) groups can optionally be substituted, for example, with C₁₋₈ alkyl, methylformyl, or −SO₃H. In certain embodiments, the alkyl groups are unsubstituted.

In certain embodiments, the primary amine compound is selected from the group consisting of

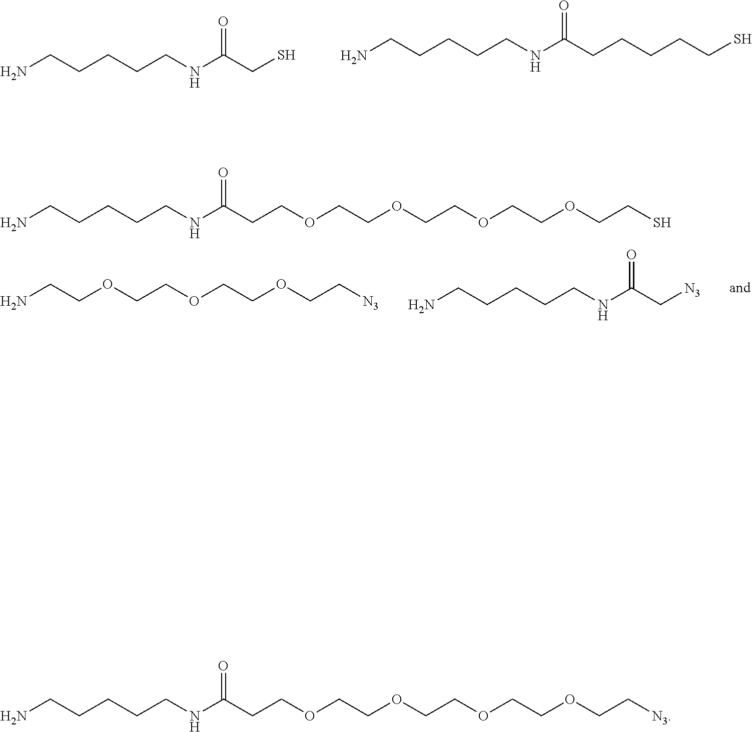

In particular embodiments, the primary amine compound is

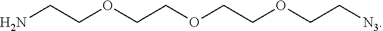 [SMILES: [N-]=[N+]=NCCOCCOCCOCCN]

Exemplary conditions for the above reactions are provided in the Examples below.

### Linkers

In certain embodiments, the linker L portion of the conjugates described herein is a moiety, for instance a divalent moiety, that covalently links a binding agent to a payload compound described herein. In other instances, the linker L is a trivalent or multivalent moiety that covalently links a binding agent to a payload compound described herein. Suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference. In certain embodiments, the linker L portion of the linker-payloads or linker-prodrug payloads described herein is a moiety covalently linked to a payload or prodrug payload compound described herein, capable of divalently and covalently linking a binding agent to a payload or prodrug payload compound described herein. In other instances, the linker L portion of the linker-payloads described herein is a moiety covalently linked to a payload or prodrug payload compound described herein, capable of covalently linking, as a trivalent or multivalent moiety, a binding agent to a payload or prodrug payload compound described herein. Payload or prodrug payload compounds include compounds of Formulae I, Ia, Iaa, II, III, IV, V, and VI above, and their residues following bonding or incorporation with linker L are linker-payloads or linker-prodrug payloads. The linker-payloads can be further bonded to binding agents such as antibodies or antigen binding fragments thereof to form antibody-drug conjugates. Those of skill in the art will recognize that certain functional groups of payload moieties are convenient for linking to linkers and/or binding agents. For example, in certain embodiments, the linker is absent and payloads or prodrug payloads are directly bonded to binding agents. In one embodiment, payloads or prodrug payloads include terminal alkynes and binding agents include azides, where each alkyne and azide participate in regioisomeric click chemistry to bind payload or prodrug payload residues directly to binding agent residues. In another embodiment, payloads or prodrug payloads include carboxylic acids and binding agents include lysines, where each carboxylic acid and lysine participate in amide bond formation to bind payload or prodrug payload residues directly to binding agent residues. Payload functional groups further include amines (e.g., Formulae C, D, E, LPc, LPd, and LPe), quaternary ammonium ions (e.g., Formulae A and LPa), hydroxyls (e.g., Formulae C, D, E, LPc, LPd, and LPe), phosphates, carboxylic acids (e.g., in the form of esters upon linking to L, as in Formulae B, D, LPb, and LPd), hydrazides (e.g., Formulae B and LPb), amides (e.g., derived from anilines of Formula C and LPc, or amines of Formulae D, E, LPd, and LPe), and sugars.

In certain embodiments, the linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds (i.e., distinguished from prodrug payloads having peptide bonds, as described elsewhere herein), ester linkages, hydrazones, β-glucuronide linkages, and disulfide linkages. In some embodiments, the linker comprises a cathepsin-cleavable linker. In some embodiments, the linker comprises a β-glucuronidase (GUSB)-cleavable linker (see, e.g., GUSB linkers from Creative Biolabs, creative-biolabs.com/adc/beta-glucuronide-linker.htm, or ACS Med. Chem. Lett. 2010, 1: 277-280).

In some embodiments, the linker comprises a non-cleavable moiety. In some embodiments, the non-cleavable linker is derived from

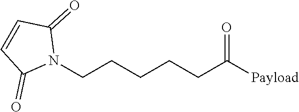

or a residue thereof. In some embodiments, the non-cleavable linker-payload residue is

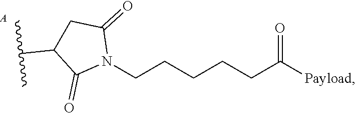

or a regioisomer thereof. In some embodiments, the non-cleavable linker is derived from

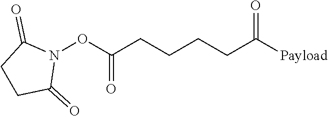

or a residue thereof. In some embodiments, the non-cleavable linker-payload residue is

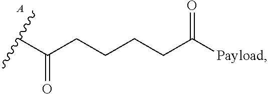

or a regioisomer thereof. In one embodiment, the linker is maleimide cyclohexane carboxylate or 4-(N-maleimidomethyl)cyclohexanecarboxylic acid (MCC). In the structures,

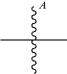

indicates a bond to a binding agent. In the structures, in some examples,

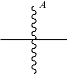

indicates a click chemistry residue which results from the reaction of, for example, a binding agent having an azide or alkyne functionality and a linker-payload having a complementary alkyne or azide functionality. In the structures, in other examples,

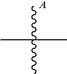

indicates a divalent sulfide which results from the reaction of, for example, one or more binding agent cysteines with one or more linkers or linker-payloads having maleimide functionality via Michael addition reactions. In the structures, in other examples,

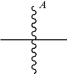

indicates an amide bond which results from the reaction of, for example, one or more binding agent lysines with one or more linkers or linker-payloads having activated or unactivated carboxyl functionality, as would be appreciated by a person of skill in the art. In one embodiment,

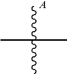

indicates an amide bond which results from the reaction of, for example, one or more binding agent lysines with one or more linkers or linker-payloads having activated carboxyl functionality, as would be appreciated by a person of skill in the art.

In some embodiments, suitable linkers include, but are not limited to, those that are chemically bonded to two cysteine residues of a single binding agent, for example, antibody. Such linkers can serve to mimic the antibody's disulfide bonds that are disrupted as a result of the conjugation process.

In some embodiments, the linker comprises one or more amino acids (i.e., distinguished from prodrug payloads comprising peptide bonds derived from distinguishable amino acids, as described elsewhere herein). Suitable amino acids include natural, non-natural, standard, non-standard, proteinogenic, non-proteinogenic, and L- or D-α-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combination thereof (e.g., dipeptides, tripeptides, oligopeptides, polypeptides, and the like). In certain embodiments, one or more side chains of the amino acids are linked to a side chain group, described below. In some embodiments, the linker is a peptide comprising or consisting of the amino acids valine and citrulline (e.g., divalent -Val-Cit- or divalent -VCit-). In some embodiments, the linker is a peptide comprising or consisting of the amino acids alanine and alanine, or divalent -AA-. In some embodiments, the linker is a peptide comprising or consisting of the amino acids glutamic acid and alanine, or -EA-. In some embodiments, the linker is a peptide comprising or consisting of the amino acids glutamic acid and glycine, or -EG-. In some embodiments, the linker is a peptide comprising or consisting of the amino acids glycine and glycine, or -GG -. In some embodiments, the linker is a peptide comprising or consisting of the amino acids glutamine, valine, and citrulline, or -Q-V-Cit- or -QVCit -. In some embodiments, the linker is a peptide comprising or consisting of the amino acids glutamic acid, valine, and citrulline, or -E-V-Cit- or -EVCit -. In some embodiments, the linker is a peptide comprising or consisting of the amino acids -GGGGS- (SEQ ID NO: 18). In some embodiments, the linker is a peptide comprising or consisting of the amino acids -GGGGG- (SEQ ID NO: 19). In some embodiments, the linker is a peptide comprising or consisting of the amino acids -GGGGK- (SEQ ID NO: 20). In some embodiments, the linker is a peptide comprising or consisting of the amino acids -GFGG- (SEQ ID NO: 21). In some embodiments, the linker is a peptide comprising or consisting of the amino acids -GG -. In some embodiments, the linker is a peptide comprising or consisting of the amino acids -GGG -. In some embodiments, the linker is a peptide comprising or consisting of the amino acids -GGGG- (SEQ ID NO: 22). In some embodiments, the linker is a peptide comprising or consisting of the amino acids -GGFG- (SEQ ID NO: 23). In some embodiments, the linker is a peptide comprising or consisting of the amino acids lysine, valine, and citrulline, or -KVCit -. In some embodiments, the linker is a peptide comprising or consisting of the amino acids -KVA -. In some embodiments, the linker is a peptide comprising or consisting of the amino acids -VA -. In any of the embodiments in this paragraph, and throughout this disclosure, the standard three-letter or one-letter amino acid designations are used, as would be appreciated by a person of skill in the art. Exemplary single-letter amino acid designations include, G for glycine, K for lysine, S for serine, V for valine, A for alanine, and F for phenylalanine.

In some embodiments, the linker comprises a self-immolative group. The self-immolative group can be any such group known to those of skill. In particular embodiments, the self-immolative group is p-aminobenzyl (PAB), or a derivative thereof. Useful derivatives include p-aminobenzyloxycarbonyl (PABC). Those of skill will recognize that a self-immolative group is capable of carrying out a chemical reaction, which releases the remaining atoms of a linker from a payload.

In some embodiments, the linker is

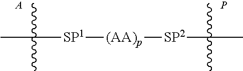

wherein
SP¹ is a spacer;
SP² is a spacer;

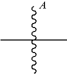

is one or more bonds to the binding agent;

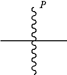

is one or more bonds to the payload;
each AA is an amino acid residue; and
p is an integer from zero to ten.
   In certain embodiments, each AA here within the linker L can be characterized as a second amino acid residue, in contrast to a first amino acid residue within a payload or prodrug payload, as described elsewhere herein. As would be appreciated by a person of skill in the art, in certain embodiments, more than one AA here within the linker L can be characterized as a second peptide residue, in contrast to a first peptide residue within a payload or prodrug payload, as described elsewhere herein.

The SP¹ spacer is a moiety that connects the (AA)ₚ moiety or residue to the binding agent (BA) or to a reactive group residue which is bonded to BA. Suitable SP¹ spacers include, but are not limited to, those comprising alkylene or polyether, or both. The ends of the spacers, for example, the portion of the spacer bonded to the BA or an AA, can be moieties derived from reactive moieties that are used for purposes of coupling the antibody or an AA to the spacer during chemical synthesis of the conjugate. In certain embodiments, p is zero, one, two, three, or four. In particular embodiments, p is 2. In particular embodiments, p is 3. In particular embodiments, p is 4.

In some embodiments, the SP¹ spacer comprises an alkylene. In some embodiments, the SP¹ spacer comprises a C₅₋₇ alkylene. In some embodiments, the SP¹ spacer comprises a polyether. In some embodiments, the SP¹ spacer comprises a polymer of ethylene oxide such as polyethylene glycol.

In some embodiments, the SP¹ spacer is

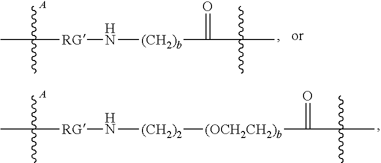

wherein
RG′ is a reactive group residue following reaction of a reactive group RG with a binding agent,

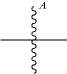

is a bond to the binding agent;

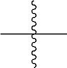

is a bond to (AA)ₚ where p is an integer from zero to ten; and
b is an integer from two to eight.

The reactive group RG can be any reactive group known to those of skill in the art to be capable of forming one or more bonds to the binding agent. The reactive group RG is a moiety comprising a portion in its structure that is capable of reacting with the binding agent (e.g., reacting with an antibody at its cysteine or lysine residues, or at an azide moiety, for example, a PEG-N3 functionalized antibody at one or more glutamine residues) to form a compound of Formula A, A′, B, B′, C, C′, D, D′, E, or E′. Following conjugation to the binding agent, the reactive group becomes the reactive group residue (RG′). Illustrative reactive groups include, but are not limited to, those that comprise haloacetyl, isothiocyanate, succinimide, N-hydroxysuccinimide, or maleimide portions that are capable of reacting with the binding agent.

In certain embodiments, reactive groups include, but are not limited to, alkynes. In certain embodiments, the alkynes are alkynes capable of undergoing 1,3-cycloaddition reactions with azides in the absence of copper catalysts, such as strained alkynes. Strained alkynes are suitable for strain-promoted alkyne-azide cycloadditions (SPAAC), and include cycloalkynes, for example, cyclooctynes and benzannulated alkynes. Suitable alkynes include, but are not limited to, dibenzoazacyclooctyne or

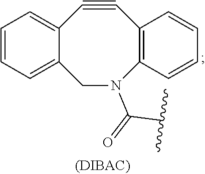 [SMILES: *C(=O)N1Cc2ccccc2C#Cc2ccccc21]

dibenzocyclooctyne or

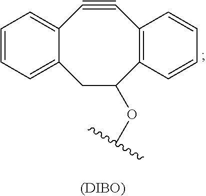 [SMILES: *OC1Cc2ccccc2C#Cc2ccccc21 ; "(DIBO)"]

biarylazacyclooctynone or

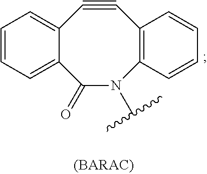 [SMILES: *N1C(=O)c2ccccc2C#Cc2ccccc21]

difluorinated cyclooctyne or

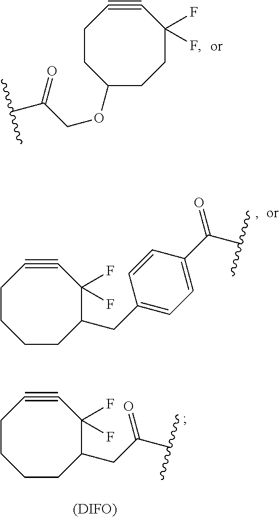

substituted, for example, fluorinated alkynes, aza-cycloalkynes, bicycle[6.1.0]nonyne or

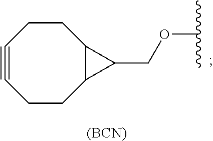

and derivatives thereof. Particularly useful alkynes include

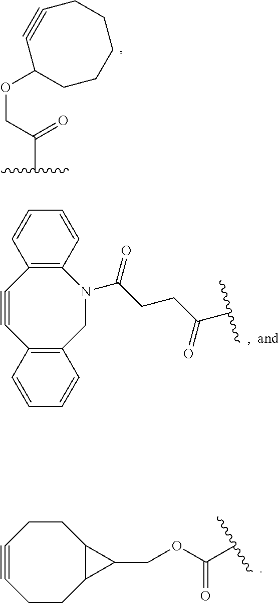

In certain embodiments, the binding agent is bonded directly to RG′. In certain embodiments, the binding agent is bonded to RG′ via a spacer, for instance SP⁴, located between

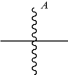

and RG′. In particular embodiments, the binding agent is bonded indirectly to RG′ via SP⁴, for example, a PEG spacer. As discussed in detail below, in certain embodiments, the binding agent is prepared by functionalizing with one or more azido groups. Each azido group is capable of reacting with RG to form RG′. In particular embodiments, the binding agent is derivatized with —PEG-N₃ linked to a glutamine residue (e.g., a transglutaminse-modified binding agent). Exemplary —N₃ derivatized binding agents, methods for their preparation, and methods for their use in reacting with RG are provided herein. In certain embodiments, RG is an alkyne suitable for participation in 1,3-cycloadditions, and RG′ is a regioisomeric 1,2,3-triazolyl moiety formed from the reaction of RG with an azido-functionalized binding agent. By way of further example, in certain embodiments, RG′ is linked to the binding agent as shown in

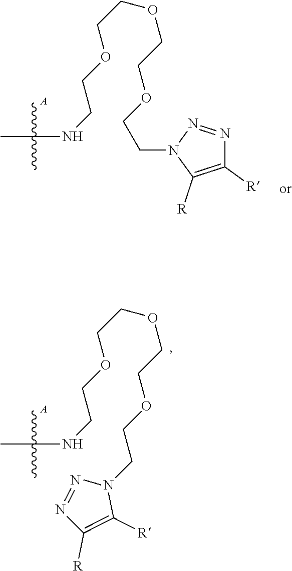

or a mixture of each regioisomer. Each R and R′ is as described or exemplified herein.

The SP² spacer, when present, is a moiety that connects the (AA)ₚ moiety to the payload. Suitable spacers include, but are not limited to, those described above as SP′ spacers. Further suitable SP² spacers include, but are not limited to, those comprising alkylene or polyether, or both. The ends of the SP² spacers, for example, the portion of the spacer directly bonded to the payload, prodrug payload, or an AA, can be moieties derived from reactive moieties that are used for purposes of coupling the payload, prodrug payload, or AA to the SP² spacer during the chemical synthesis of the conjugate. In some examples, the ends of the SP² spacers, for example, the portion of the SP² spacer directly bonded to the payload, prodrug payload, or an AA, can be residues of reactive moieties that are used for purposes of coupling the payload, prodrug payload, or an AA to the spacer during the chemical synthesis of the conjugate.

In some embodiments, the SP² spacer, when present, is selected from the group consisting of —NH-(p-C₆H₄)—CH₂—, —NH-(p-C₆H₄)—CH₂OC(O)—, an amino acid, a dipeptide, a tripeptide, an oligopeptide, —O—, —N(H)—,

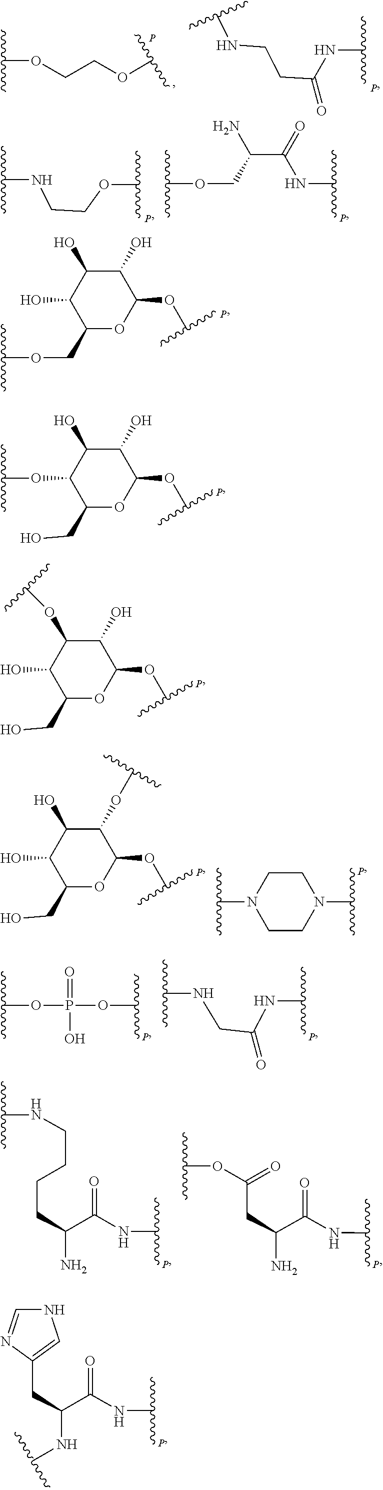

and any combinations thereof. In certain embodiments, each

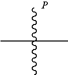

is a bond to the payload or prodrug payload, and each

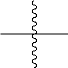

is a bond to (AA)ₚ.

In the above formulas, each (AA)ₚ is an amino acid or, optionally, a p-aminobenzyloxycarbonyl residue (PABC),

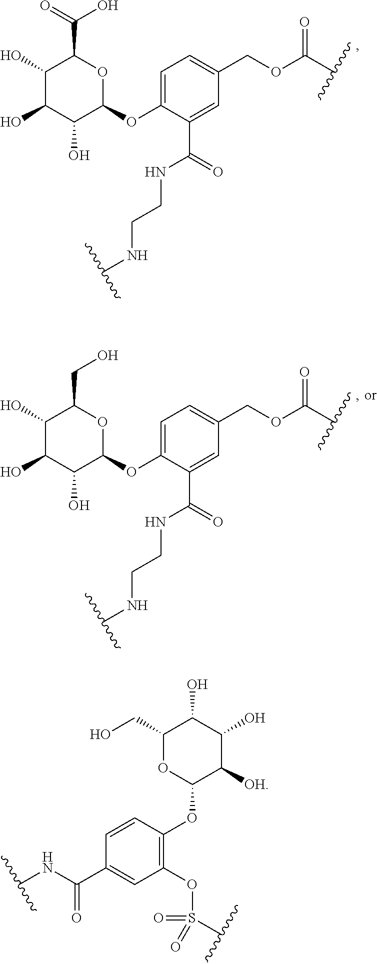 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O ; *NC(=O)c1ccc(O[C@@H]2O[C@H](CO)[C@H](O)[C@H](O)[C@H]2O)c(OS(*)(=O)=O)c1]

If PABC is present, then in particular embodiments only one PABC is present. In certain embodiments, the PABC residue, if present, is bonded to a terminal AA in the (AA)ₚ group, proximal to the payload or prodrug payload. If

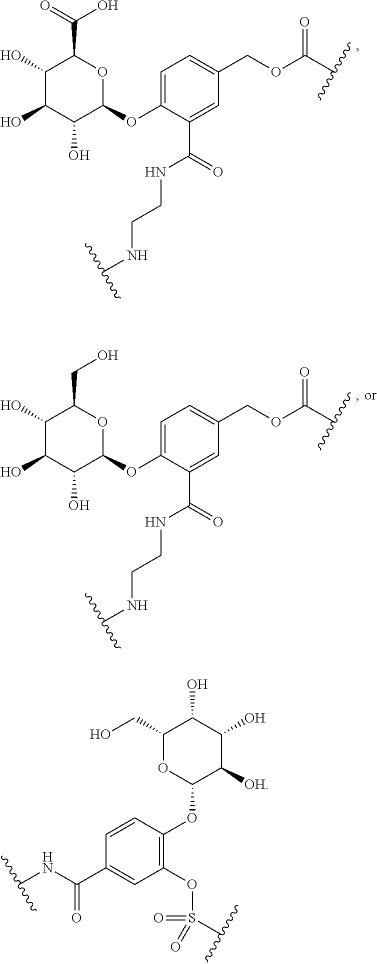 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O ; *NC(=O)c1ccc(O[C@@H]2O[C@H](CO)[C@H](O)[C@H](O)[C@H]2O)c(OS(*)(=O)=O)c1]

is present, then only

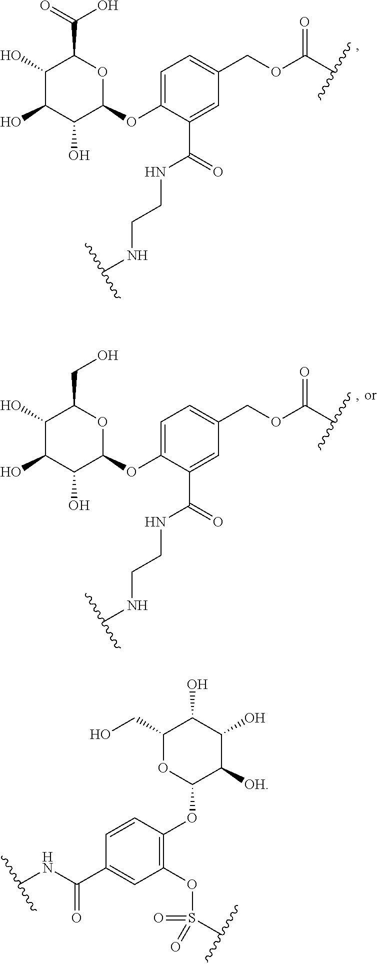 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O ; *NC(=O)c1ccc(O[C@@H]2O[C@H](CO)[C@H](O)[C@H](O)[C@H]2O)c(OS(*)(=O)=O)c1]

is present. In certain embodiments, the

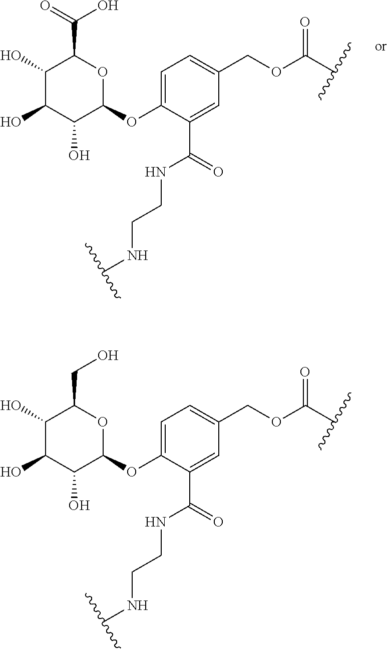 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

residue, if present, is bonded to the payload or prodrug payload via the benzyloxycarbonyl moiety, and no AA is present. In certain embodiments, the

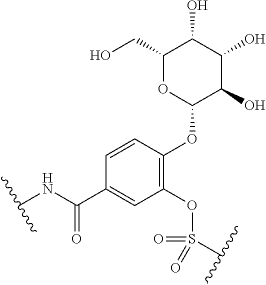 [SMILES: *NC(=O)c1ccc(O[C@@H]2O[C@H](CO)[C@H](O)[C@H](O)[C@H]2O)c(OS(*)(=O)=O)c1]

residue, if present, is bonded to the payload or prodrug payload via —O—. Suitable amino acids for each AA include natural, non-natural, standard, non-standard, proteinogenic, non-proteinogenic, and L- or D-α-amino acids. In some embodiments, the AA comprises alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combinations thereof (e.g., dipeptides, tripeptides, and oligopeptides, and the like). In certain embodiments, one or more side chains of the amino acids is linked to a side chain group, described below. In some embodiments, p is two. In some embodiments, the (AA)ₚ is valine-citrulline. In some embodiments, (AA)ₚ is citrulline-valine. In some embodiments, (AA)ₚ is valine-alanine. In some embodiments, (AA)ₚ is alanine-valine. In some embodiments, (AA)ₚ is valine-glycine. In some embodiments, (AA)ₚ is glycine-valine. In some embodiments, p is three. In some embodiments, the (AA)ₚ is valine-citrulline-PABC. In some embodiments, (AA)ₚ is citrulline-valine-PABC. In some embodiments, (AA)ₚ is glutamate-valine-citrulline. In some embodiments, (AA)ₚ is glutamine-valine-citrulline. In some embodiments, (AA)ₚ is lysine-valine-alanine. In some embodiments, (AA)ₚ is lysine-valine-citrulline. In some embodiments, p is four. In some embodiments, (AA)ₚ is glutamate-valine-citrulline-PABC. In some embodiments, (AA)ₚ is glutamine-valine-citrulline-PABC. Those of skill will recognize PABC as a residue of p-aminobenzyloxycarbonyl with the following structure

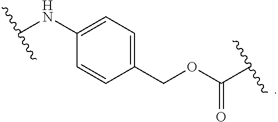 [SMILES: *Nc1ccc(COC(*)=O)cc1]

The PABC residue has been shown to facilitate cleavage of certain linkers in vitro and in vivo. Those of skill will recognize PAB as a divalent residue of p-aminobenzyl or —NH-(p-C₆H₄)—CH₂—.

In some embodiments, the linker is

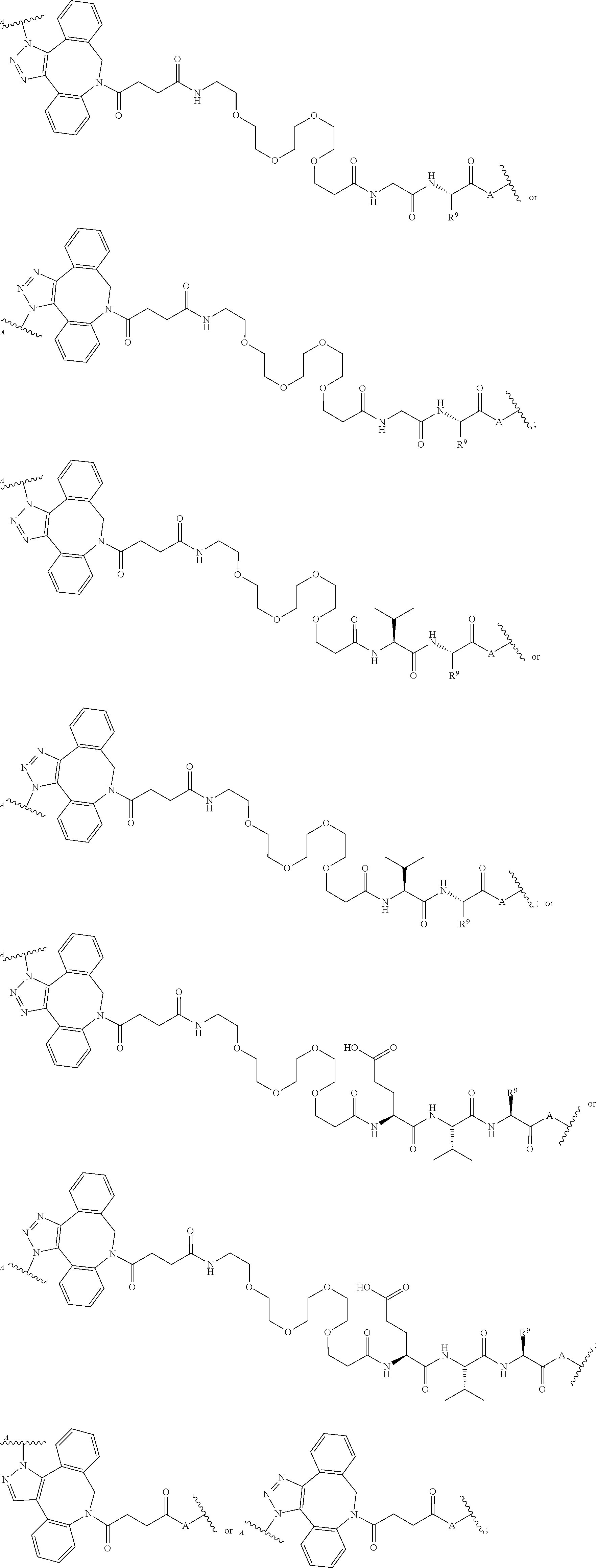

wherein
each

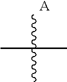

is a bond to a transglutaminase-modified binding agent;
each

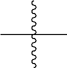

is a bond to the payload;
each R⁹ is —CH₂Ph or —(CH₂)₃N(H)C(O)NH₂; and
each A is a bond, —NH—,

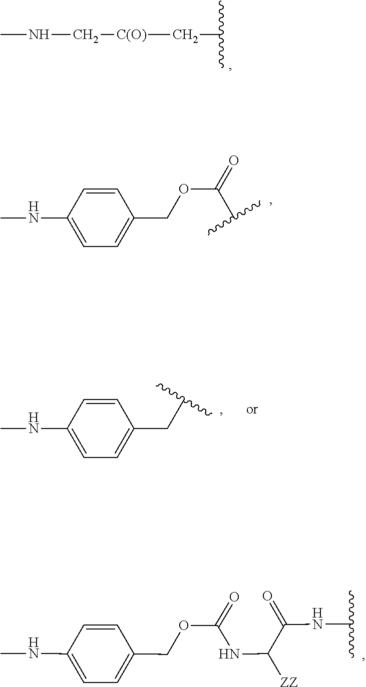

where ZZ is hydrogen, or a side chain for an amino acid as discussed elsewhere herein. By way of further example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

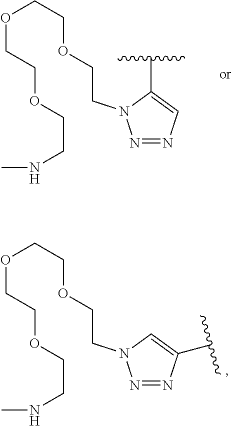 [SMILES: *c1cnnn1CCOCCOCCOCCNC ; *c1cn(CCOCCOCCOCCNC)nn1]

or a mixture thereof. Alternatively, in another embodiment, A is

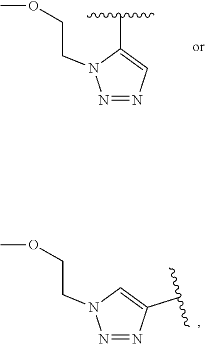

or a mixture thereof. In another embodiment, A is

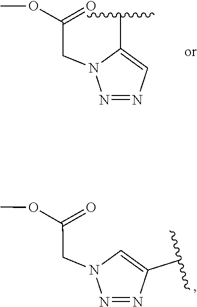 [SMILES: *c1cnnn1CC(=O)OC ; *c1cn(CC(=O)OC)nn1]

or a mixture thereof. In another embodiment, A is

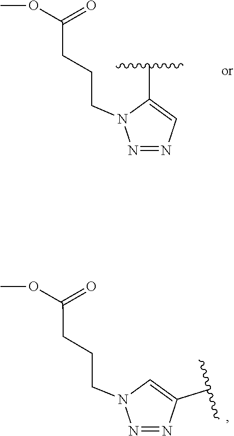 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is

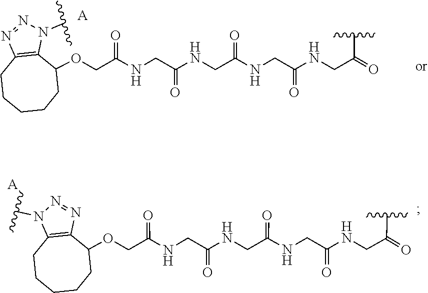

wherein
each

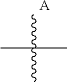

is a bond to a transglutaminse-modified binding agent; and
each

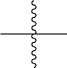

is a bond to the payload. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

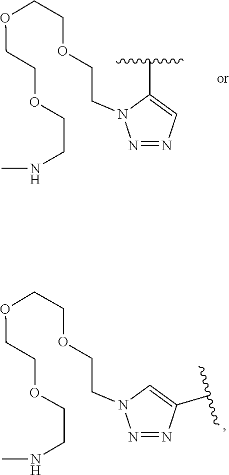 [SMILES: *c1cnnn1CCOCCOCCOCCNC ; *c1cn(CCOCCOCCOCCNC)nn1]

or a mixture thereof. Alternatively, in another embodiment, A is

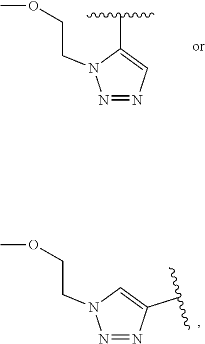

or a mixture thereof. In another embodiment, A is

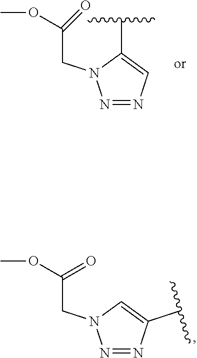 [SMILES: *c1cnnn1CC(=O)OC ; *c1cn(CC(=O)OC)nn1]

or a mixture thereof. In another embodiment, A is

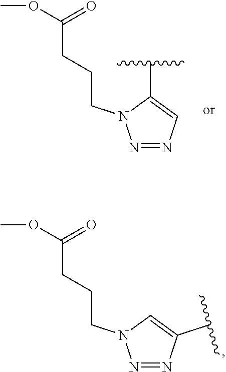 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is

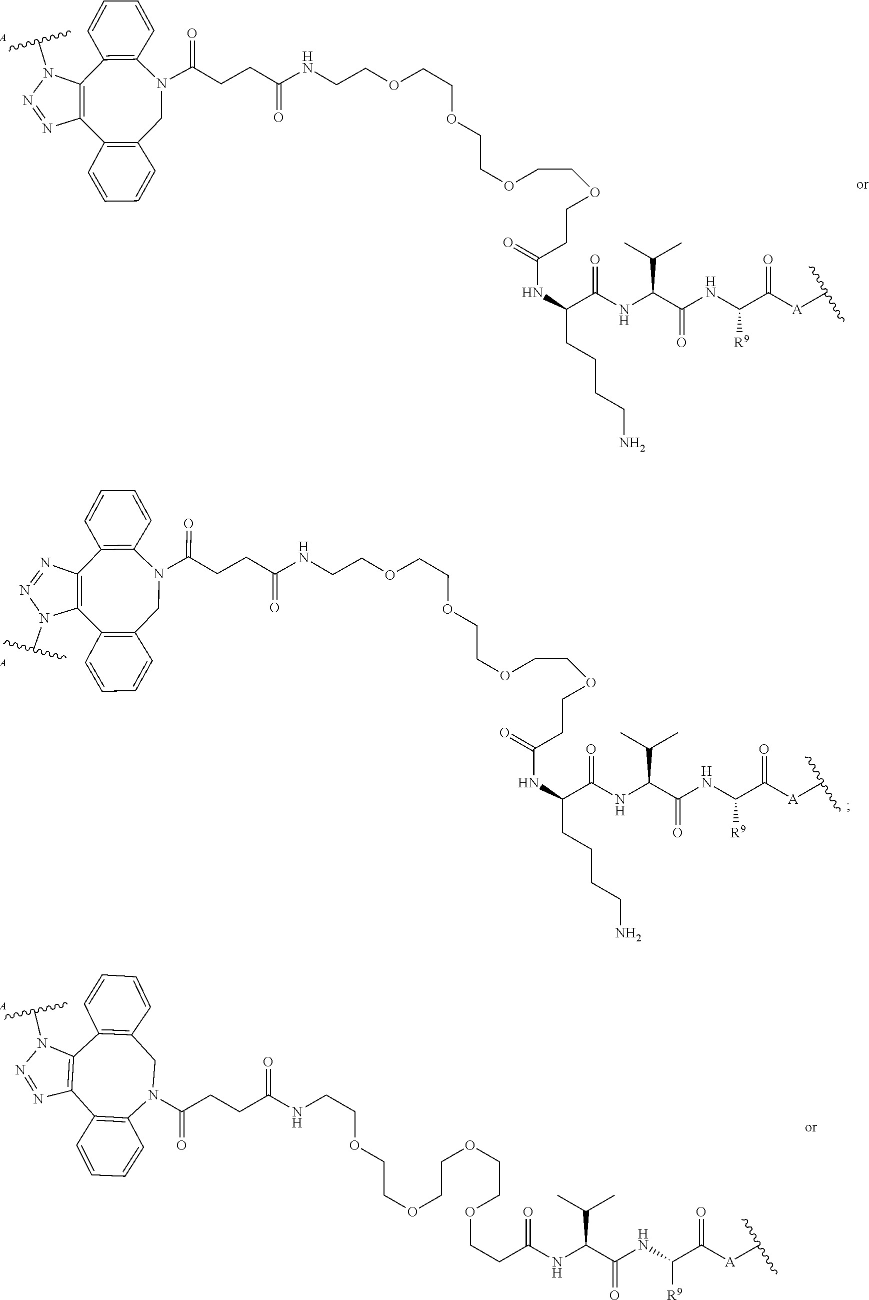

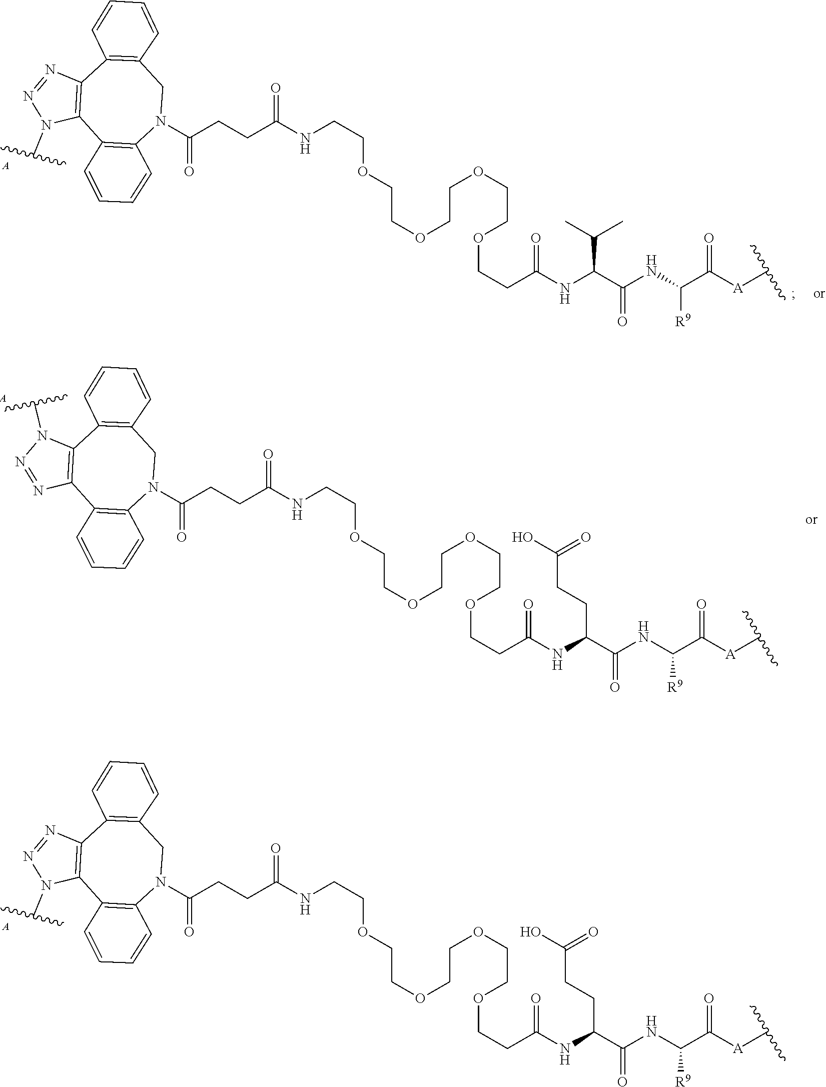

wherein:
each

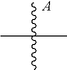

is a bond to a transglutaminase-modified binding agent;
each

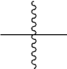

is a bond to the payload;
each R⁹ is —CH₃ or —(CH₂)₃N(H)C(O)NH₂; and
each A is —O—, —NH—,

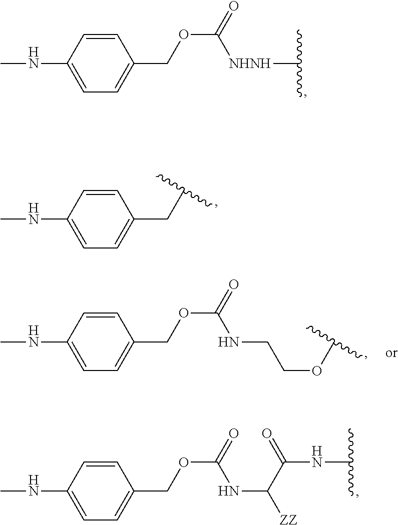

where ZZ is hydrogen, or a side chain for an amino acid as discussed elsewhere herein. By way of further example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

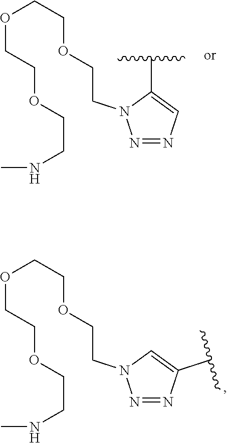 [SMILES: *c1cnnn1CCOCCOCCOCCNC ; *c1cn(CCOCCOCCOCCNC)nn1]

or a mixture thereof. Alternatively, in another embodiment, A is

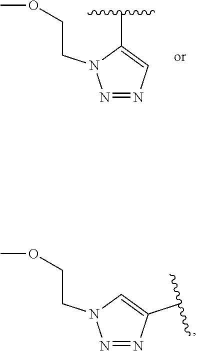

or a mixture thereof. In another embodiment, A is

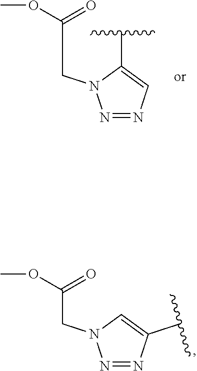 [SMILES: *c1cnnn1CC(=O)OC ; *c1cn(CC(=O)OC)nn1]

or a mixture thereof. In another embodiment, A

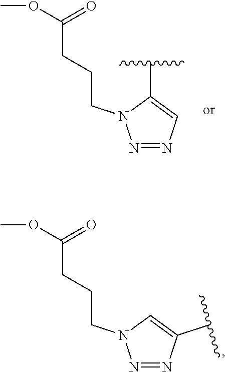 [SMILES: *c1cnnn1CCCC(=O)OC ; *c1cn(CCCC(=O)OC)nn1]

is or a mixture thereof. As discussed above, the bond to the binding agent can be direct, or via a spacer. In certain embodiments, the bond to the binding agent is via a PEG spacer to a glutamine residue of the binding agent.

In some embodiments, the linker is

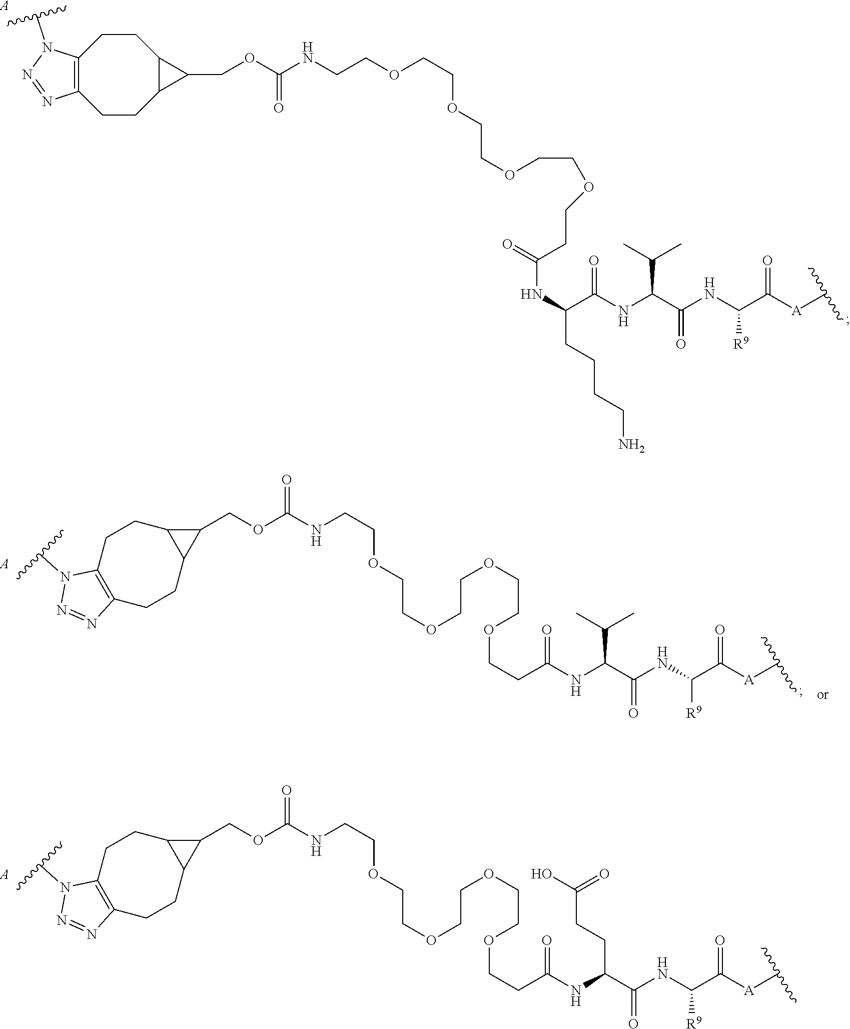

wherein
each

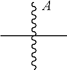

is a bond to a transglutaminse-modified binding agent;
each

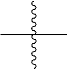

is a bond to the payload;
each R⁹ is —CH₃ or —(CH₂)₃N(H)C(O)NH₂; and
each A is —O—, —N(H)—,

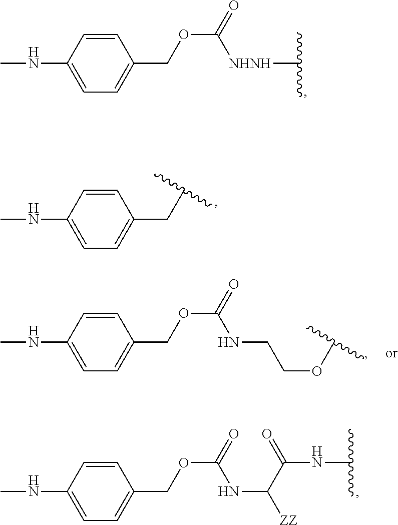

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

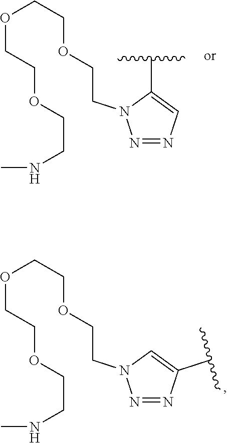 [SMILES: *c1cnnn1CCOCCOCCOCCNC ; *c1cn(CCOCCOCCOCCNC)nn1]

or a mixture thereof. Alternatively, in another embodiment, A is

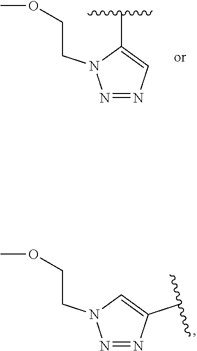

or a mixture thereof. In another embodiment, A is

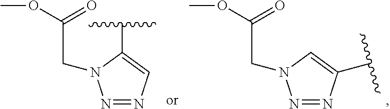 [SMILES: *c1cnnn1CC(=O)OC ; *c1cn(CC(=O)OC)nn1]

or a mixture thereof. In another embodiment, A is

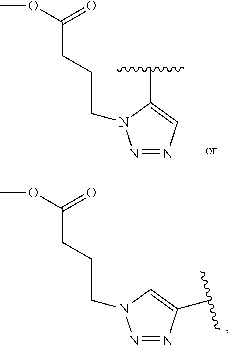 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In any of the above embodiments, the (AA)ₚ group can be modified with one or more enhancement groups. Advantageously, the enhancement group can be linked to the side chain of any amino acid in (AA)ₚ. Useful amino acids for linking enhancement groups include lysine, asparagine, aspartate, glutamine, glutamate, and citrulline. The link to the enhancement group can be a direct bond to the amino acid side chain, or the link can be indirect via a spacer and/or reactive group. Useful spacers and reactive groups include any described above. The enhancement group can be any group deemed useful by those of skill in the art. For example, the enhancement group can be any group that imparts a beneficial effect to the compound, payload, linker payload, or antibody conjugate including, but not limited to, biological, biochemical, synthetic, solubilizing, imaging, detecting, and reactivity effects, and the like. In certain embodiments, the enhancement group is a hydrophilic group. In certain embodiments, the enhancement group is a cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the enhancement group is capable of improving solublity of the remainder of the conjugate. In certain embodiments, the alkyl, heteroalkyl, alkylenyl, or heteroalkylenyl sulfonic acid is substituted or non-substituted. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ— C(O)NH—(CH₂)₁₋₅SO₃H, —(CH₂)ₙ—N(CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is one, two, three, four, or five, and m is one, two, three, four, or five. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is one, two, three, four, or five. In another embodiment, the alkyl, heteroalkyl, alkylenyl, or heteroalkylenyl sulfonic acid is —(CH₂)ₙ—C(O)NH—(CH₂)₁₋₅SO₃H, wherein n is one, two, three, four, or five. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is one, two, three, four, or five. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is one, two, three, four, or five. In some embodiments, the linker is

wherein

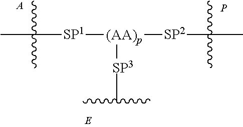

SP¹ is a spacer;
SP² is a spacer;
SP³ is a spacer, linked to one AA of (AA)ₚ;

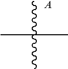

is one or more bonds to the binding agent;

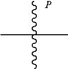

is one or more bonds to the payload or prodrug payload;

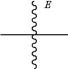

is one or more bonds to the enhancement group EG;
each AA is an amino acid; and
p is an integer from zero to ten.
   As discussed above, the bond to the binding agent can be direct, or via a spacer. In certain embodiments, the bond to the binding agent is via a PEG spacer to a glutamine residue of the binding agent.

The SP¹ spacer group is as described above. The SP² spacer group is as described above. Each (AA)ₚ group is as described above.

The SP³ spacer is a moiety that connects the (AA)ₚ moiety to the enhancement group (EG). Suitable SP³ spacers include, but are not limited to, those comprising alkylene or polyether, or both. The ends of the SP³ spacers, for instance, the portion of the SP³ spacer directly bonded to the enhancement group or an AA, can be moieties derived from reactive moieties that are used for purposes of coupling the enhancement group or an AA to the SP³ spacer during the chemical synthesis of the conjugate. In some examples, the ends of the SP³ spacers, for instance, the portion of the spacer directly bonded to the enhancement group or an AA, can be residues of reactive moieties that are used for purposes of coupling the enhancement group or an AA to the spacer during the chemical synthesis of the conjugate. In certain embodiments, SP³ is a spacer, linked to one and only one AA of (AA)ₚ. In certain embodiments, the SP³ spacer is linked to the side chain of a lysine residue of (AA)ₚ.

In some embodiments, the SP³ spacer is

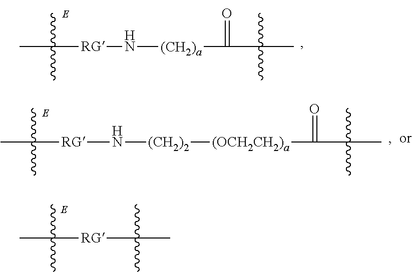

wherein
RG′ is a reactive group residue following reaction of a reactive group RG with an enhancement agent EG;

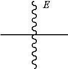

is a bond to the enhancement agent;

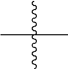

is a bond to (AA)ₚ;
a is an integer from two to eight; and
p is an integer from zero to four.

The reactive group RG can be any reactive group known to those of skill in the art to be capable of forming one or more bonds to the enhancement agent. The reactive group RG is a moiety comprising a portion in its structure that is capable of reacting with the enhancement group to form a compound of Formula LPa, LPb, LPc, LPd, LPe, LPa′, LPb′, LPc′, LPd′, LPe′, A, B, C, D, E, A′, B′, C′, D′, or E′. Following conjugation to the enhancement group, the reactive group becomes the reactive group residue (RG′). The reactive group RG can be any reactive group described above. Illustrative reactive groups include, but are not limited to, those that comprise haloacetyl, isothiocyanate, succinimide, N-hydroxysuccinimide, or maleimide portions that are capable of reacting with the binding agent.

In certain embodiments, reactive groups include, but are not limited to, alkynes. In certain embodiments, the alkynes are alkynes capable of undergoing 1,3-cycloaddition reactions with azides in the absence of copper catalysts such as strained alkynes. Strained alkynes are suitable for strain-promoted alkyne-azide cycloadditions (SPAAC), cycloalkynes, for example, cyclooctynes, and benzannulated alkynes. Suitable alkynes include, but are not limited to, dibenzoazacyclooctyne or

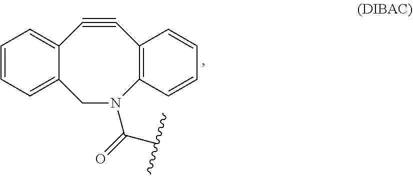 [SMILES: *C(=O)N1Cc2ccccc2C#Cc2ccccc21]

dibenzocyclooctyne or

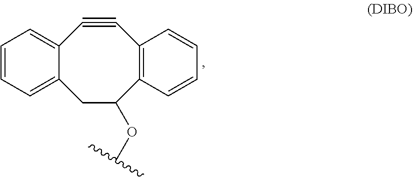 [SMILES: *OC1Cc2ccccc2C#Cc2ccccc21]

biarylazacyclooctynone or

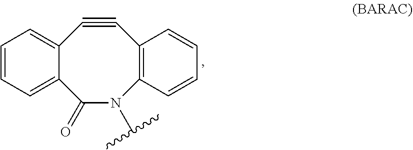 [SMILES: *N1C(=O)c2ccccc2C#Cc2ccccc21]

difluorinated cyclooctyne or

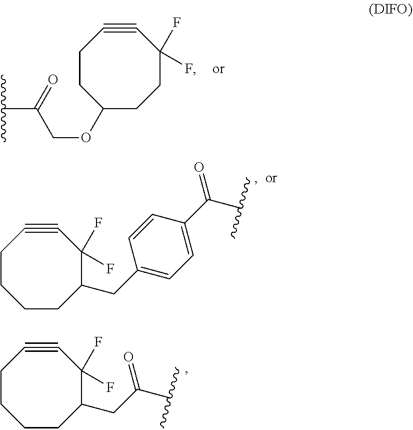

substituted, for example, fluorinated alkynes, aza-cycloalkynes, bicycle[6.1.0]nonyne or

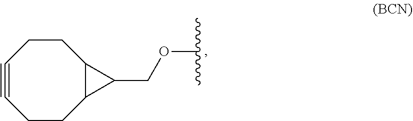 [SMILES: *OCC1C2CCC#CCCC21]

and derivatives thereof. Particularly useful alkynes include

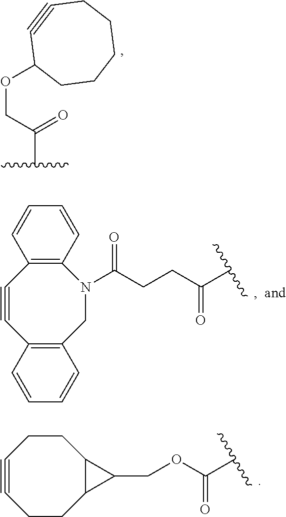

In some embodiments, the linker is

wherein

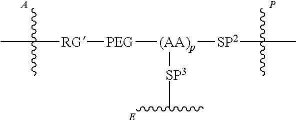

RG′ is a reactive group residue following reaction of a reactive group RG with a binding agent;
PEG is —NH—PEG4-C(O)—;
SP² is a spacer;
SP³ is a spacer, linked to one AA residue of (AA)ₚ;

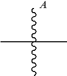

is one or more bonds to the binding agent;

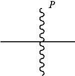

is one or more bonds to the payload;

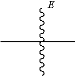

is one or more bonds to the enhancement group EG;
each AA is an amino acid residue; and
p is an integer from zero to ten.
   As discussed above, the bond to the binding agent can be direct, or via a spacer. In certain embodiments, the bond to the binding agent is via a PEG spacer to a glutamine residue of the binding agent.

In certain embodiments, the linker is

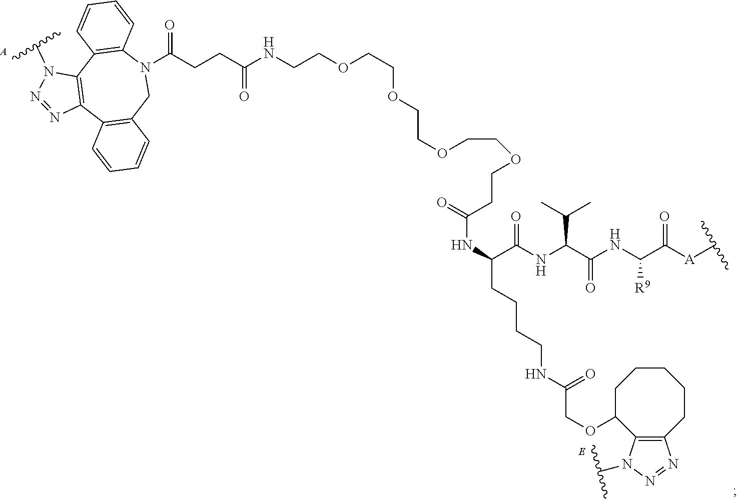

or a pharmaceutically acceptable salt, solvate, or stereoisomeric form thereof, or a regioisomer thereof, or a mixture of regioisomers thereof, wherein
each

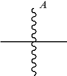

is a bond to a transglutaminase-modified binding agent;
each

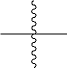

is a bond to the payload;
each

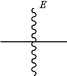

is a bond to the enhancement agent;
   each R⁹ is —CH₃ or —(CH₂)₃N(H)C(O)NH₂; and
each A is —O—, —N(H)—,

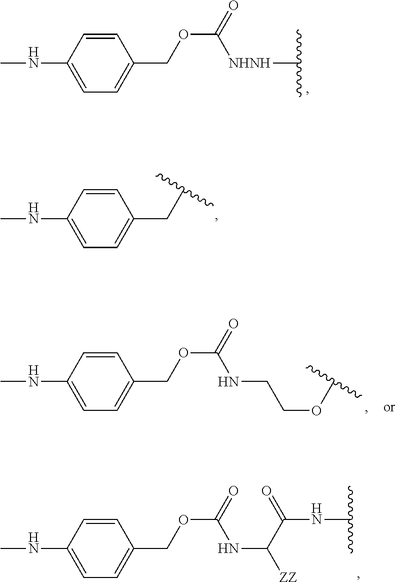

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

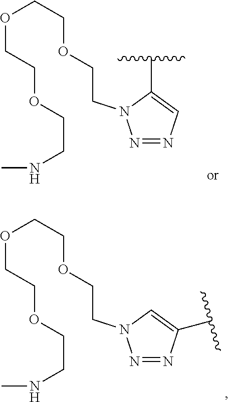 [SMILES: *c1cnnn1CCOCCOCCOCCNC ; *c1cn(CCOCCOCCOCCNC)nn1]

or a mixture thereof. Alternatively, in another embodiment, A is

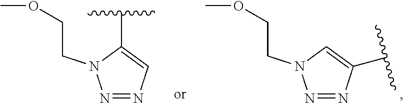

or a mixture thereof. In another embodiment, A is

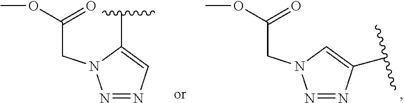 [SMILES: *c1cnnn1CC(=O)OC ; *c1cn(CC(=O)OC)nn1]

or a mixture thereof. In another embodiment, A is

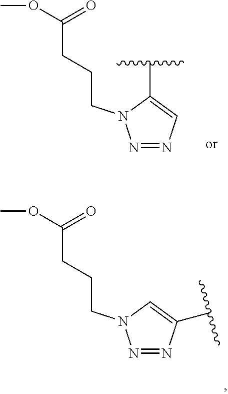 [SMILES: *c1cnnn1CCCC(=O)OC ; *c1cn(CCCC(=O)OC)nn1]

or a mixture thereof. In certain embodiments, 1,3-cycloaddition or SPAAC regioisomers, or mixture of regioisomers, are derived from PEG-N₃ derivitized antibodies treated with suitable alkynes. For example, in one embodiment, the linker is

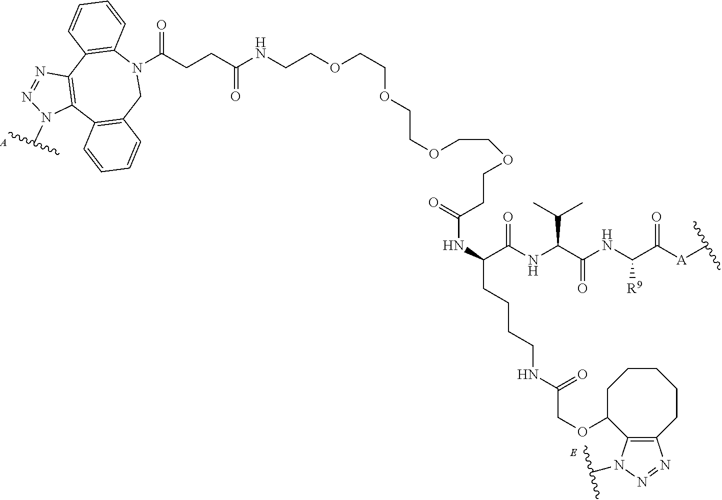

or a pharmaceutically acceptable salt, solvate, or stereoisomeric form thereof, or a regioisomer thereof, or a mixture of regioisomers thereof. By way of further example, in one embodiment, the linker is

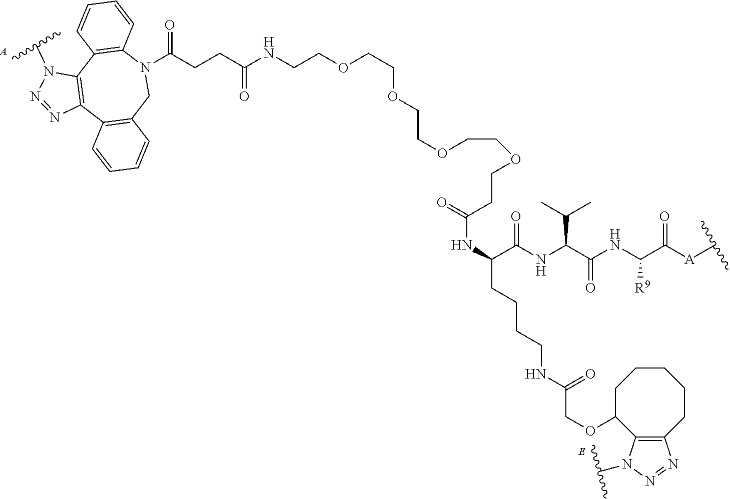

or a pharmaceutically acceptable salt, solvate, or stereoisomeric form thereof, or a regioisomer thereof, or a mixture of regioisomers thereof. By way of further example, the linker is

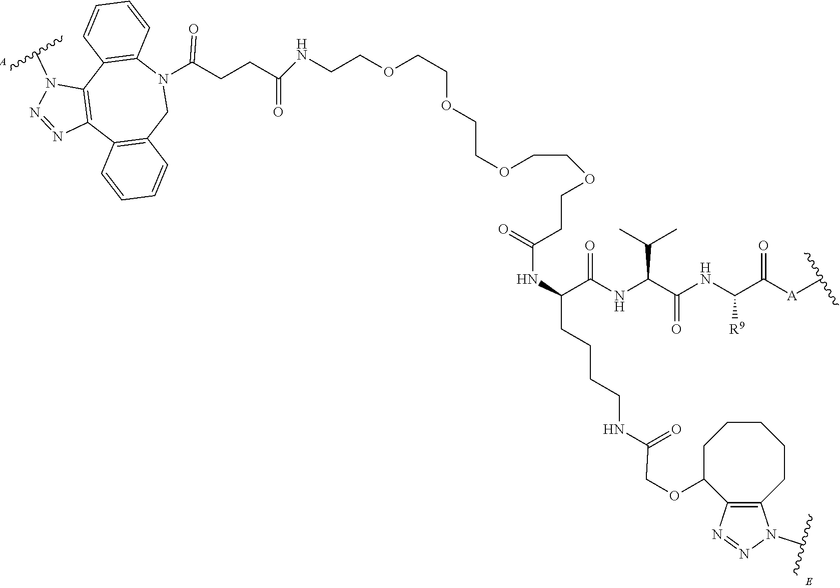

or a pharmaceutically acceptable salt, solvate, or stereoisomeric form thereof, or a regioisomer thereof, or a mixture of regioisomers thereof. By way of further example, in one embodiment, the linker is

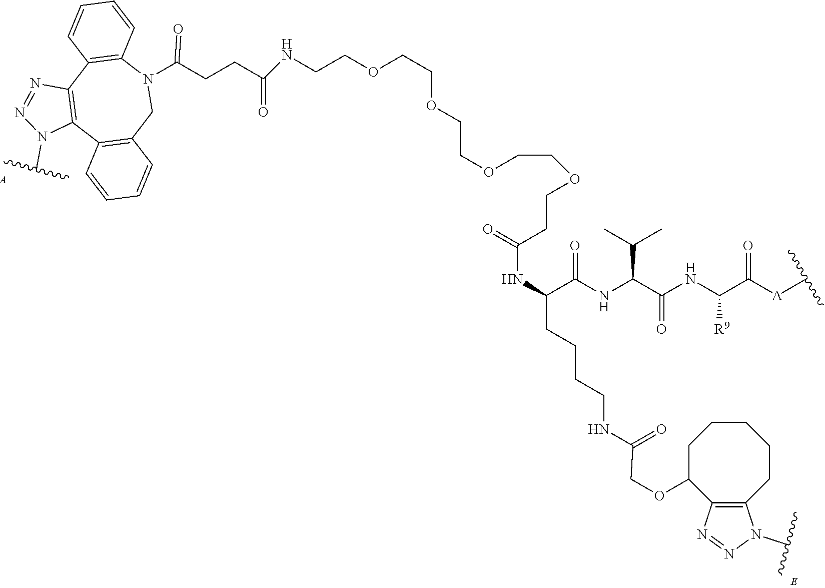

or a pharmaceutically acceptable salt, solvate, or stereoisomeric form thereof, or a regioisomer thereof, or a mixture of regioisomers thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, -(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N(CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is one, two, three, four, or five, and m is one, two, three, four, or five. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is one, two, three, four, or five. In another embodiment, the alkyl, heteroalkyl, alkylenyl, or heteroalkylenyl sulfonic acid is —(CH₂)ₙ—C(O)NH—(CH₂)₁₋₅SO₃H, wherein n is one, two, three, four, or five. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is one, two, three, four, or five. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is one, two, three, four, or five.

In some embodiments, the linker is

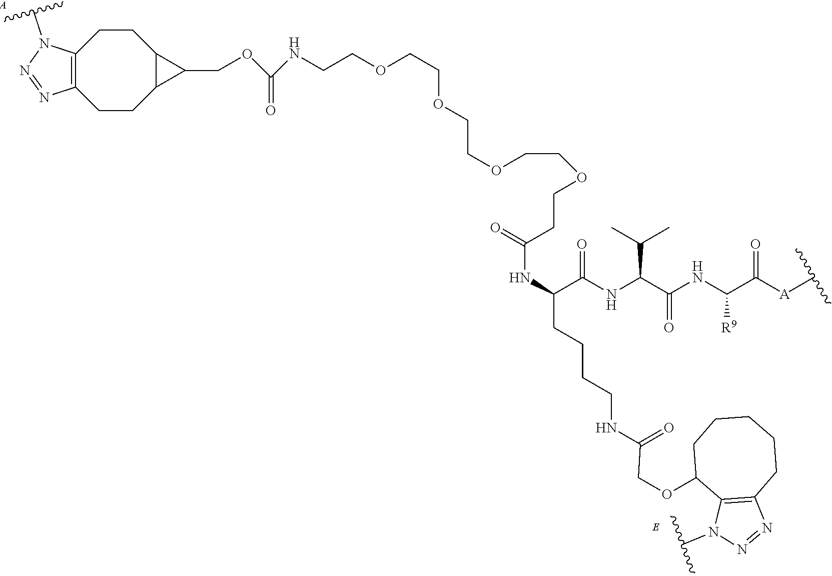

or a pharmaceutically acceptable salt, solvate, or stereoisomeric form thereof, or a regioisomer thereof, or mixture of regioisomers thereof, wherein
each

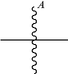

is a bond to a transglutaminase-modified binding agent;
each

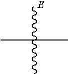

is a bond to the enhancement agent;
each

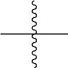

is a bond to the payload;
each R⁹ is —CH₃ or —(CH₂)₃N(H)C(O)NH₂; and
each A is —O—, —N(H)—,

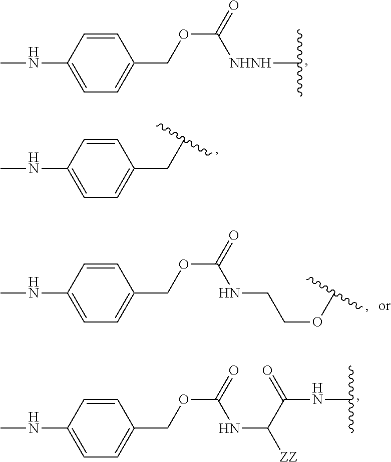

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

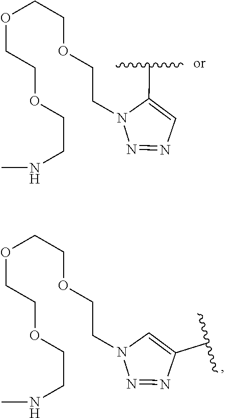 [SMILES: *c1cnnn1CCOCCOCCOCCNC ; *c1cn(CCOCCOCCOCCNC)nn1]

or a mixture thereof. Alternatively, in another embodiment, A is

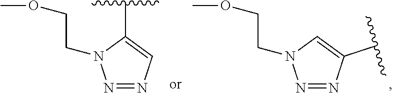

or a mixture thereof. In another embodiment, A is

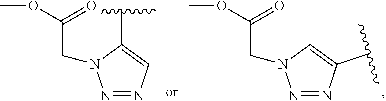 [SMILES: *c1cnnn1CC(=O)OC ; *c1cn(CC(=O)OC)nn1]

or a mixture thereof. In another embodiment, A is

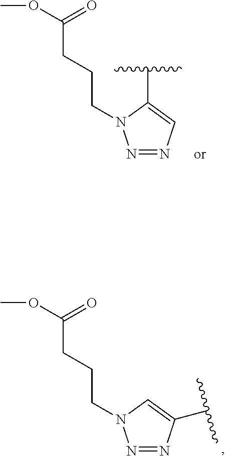 [SMILES: *c1cnnn1CCCC(=O)OC ; "or" ; *c1cn(CCCC(=O)OC)nn1 ; ","]

or a mixture thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement agent is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N(CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is one, two, three, four, or five, and m is one, two, three, four, or five. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is one, two, three, four, or five. In another embodiment, the alkyl, heteroalkyl, alkylenyl, or heteroalkylenyl sulfonic acid is —(CH₂)ₙ—C(O)NH—(CH₂)₁₋₅SO₃H, wherein n is one, two, three, four, or five. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is one, two, three, four, or five. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is one, two, three, four, or five.

In some embodiments, the linker is

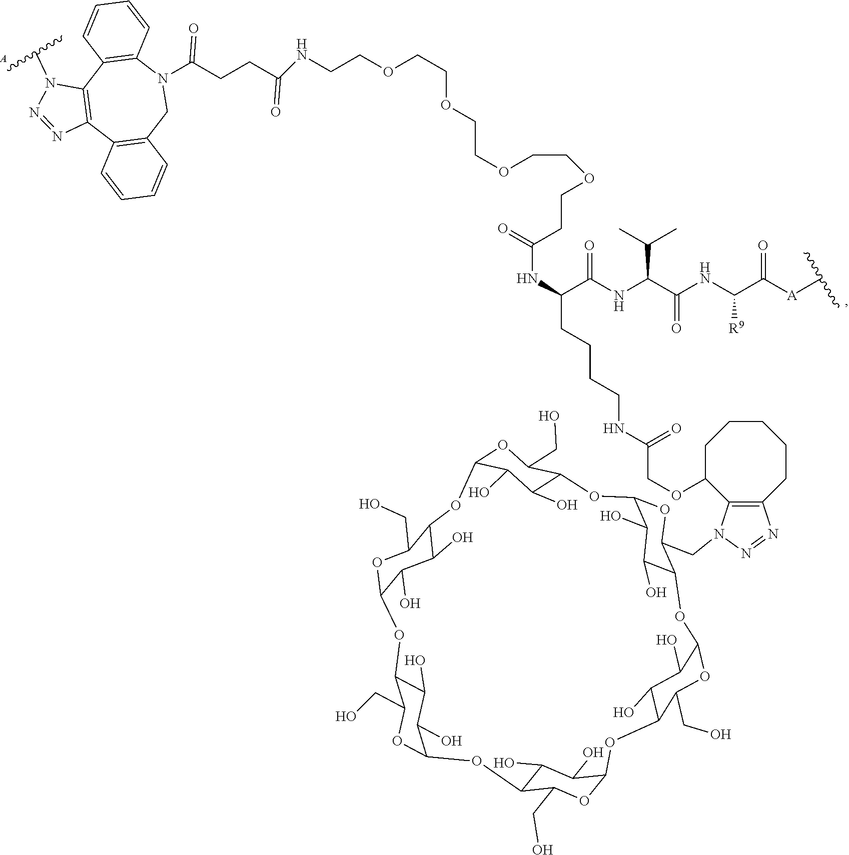

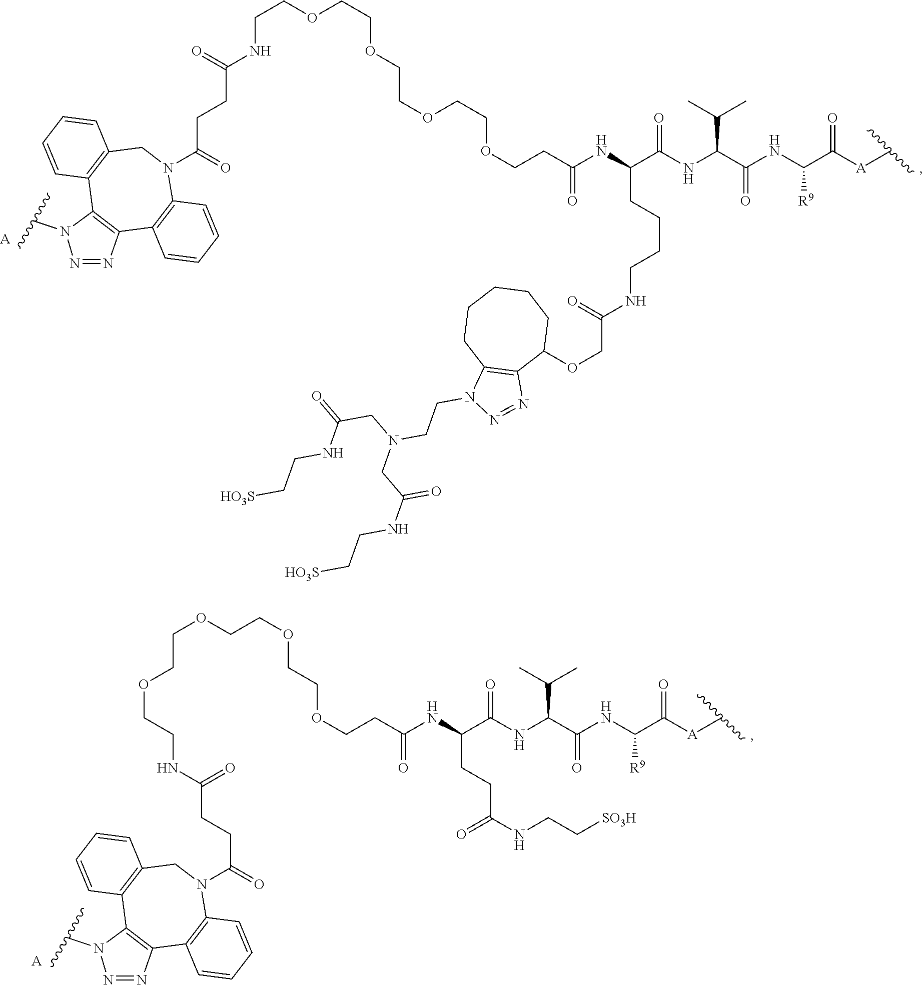

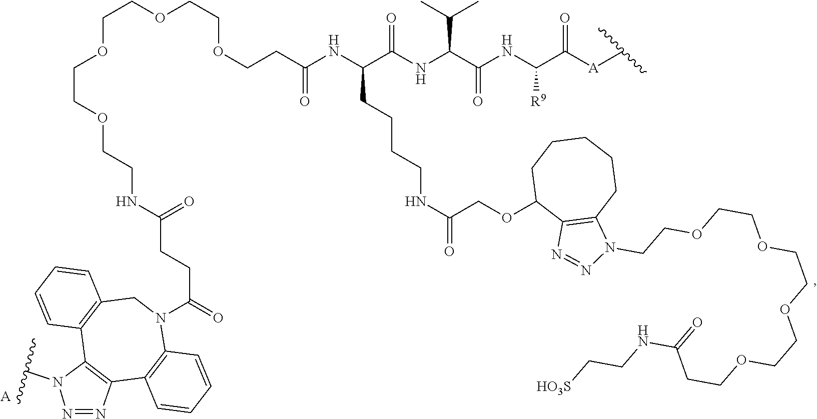

or a pharmaceutically acceptable salt, solvate, or stereoisomeric form thereof, or a regioisomer thereof, or mixture of regioisomers thereof, wherein
each

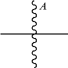

is a bond to a transglutaminse-modified binding agent;
each

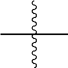

is a bond to the payload;
R⁹ is —CH₃ or —(CH₂)₃N(H)C(O)NH₂; and
A is —O—, —N(H)—,

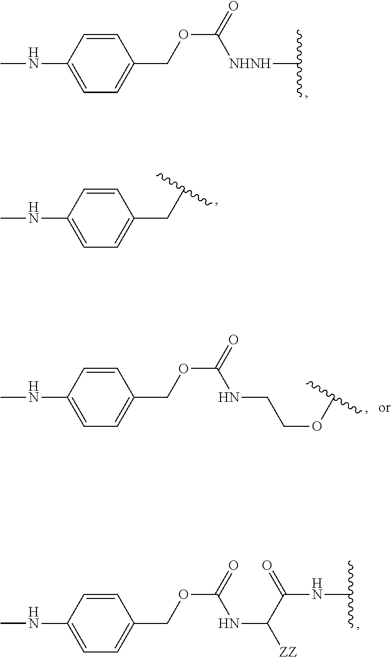

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

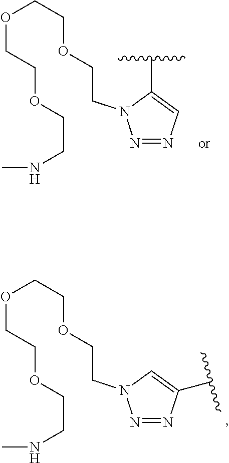 [SMILES: *c1cnnn1CCOCCOCCOCCNC ; "or" ; *c1cn(CCOCCOCCOCCNC)nn1 ; ","]

or a mixture thereof. Alternatively, in another embodiment, A is

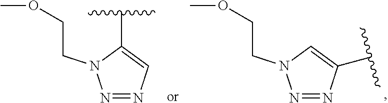

or a mixture thereof. In another embodiment, A is

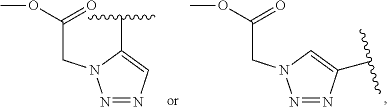 [SMILES: *c1cnnn1CC(=O)OC ; "or" ; *c1cn(CC(=O)OC)nn1 ; ","]

or a mixture thereof. In another embodiment, A is

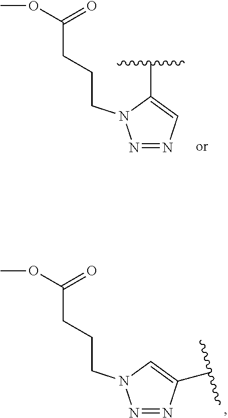 [SMILES: *c1cnnn1CCCC(=O)OC ; "or" ; *c1cn(CCCC(=O)OC)nn1 ; ","]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is

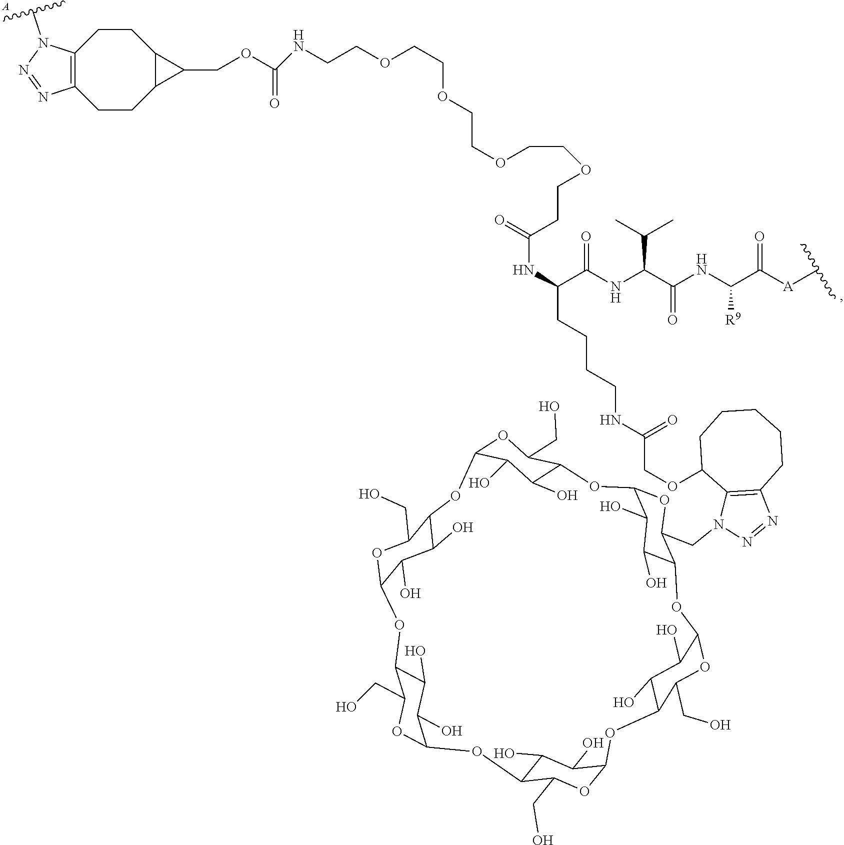

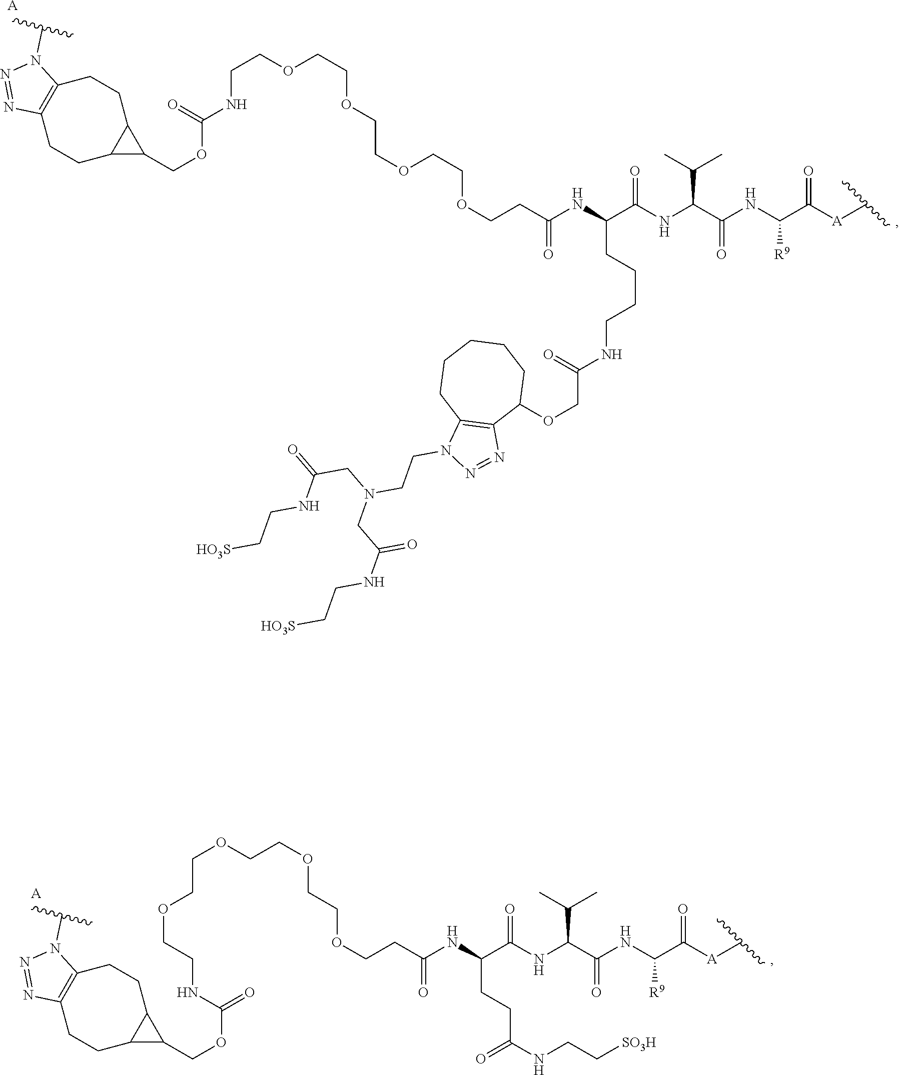

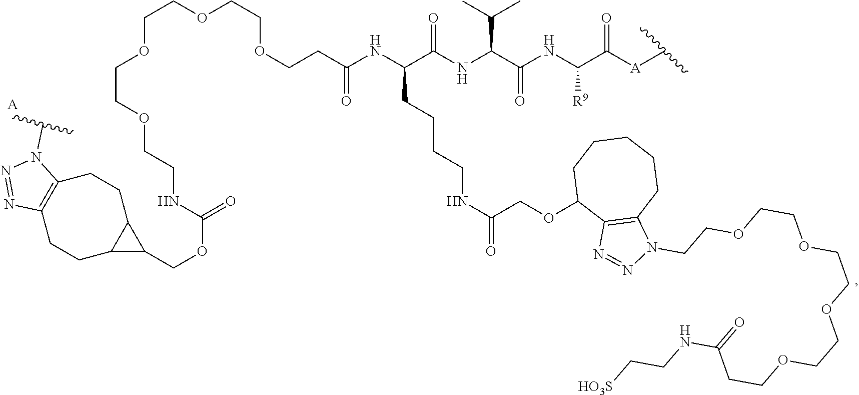

or a pharmaceutically acceptable salt, solvate, or stereoisomeric form thereof, or a regioisomer thereof, or mixture of regioisomers thereof, wherein
each

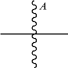

is a bond to a transglutaminse-modified binding agent;
each

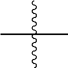

is a bond to the payload;
R⁹ is —CH₃ or —(CH₂)₃N(H)C(O)NH₂; and
A is —O—, —N(H)—,

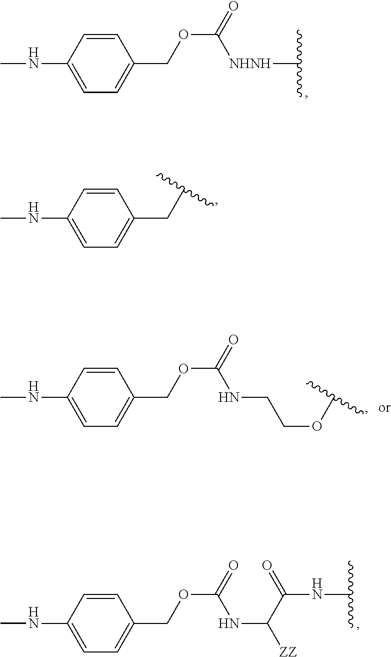

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

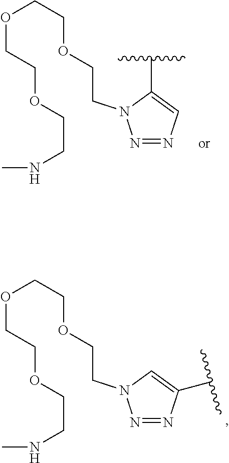 [SMILES: *c1cnnn1CCOCCOCCOCCNC ; "or" ; *c1cn(CCOCCOCCOCCNC)nn1 ; ","]

or a mixture thereof. Alternatively, in another embodiment, A is

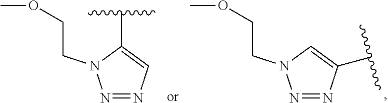

or a mixture thereof. In another embodiment, A is

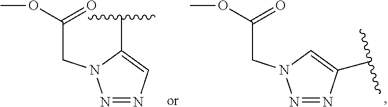 [SMILES: *c1cnnn1CC(=O)OC ; "or" ; *c1cn(CC(=O)OC)nn1 ; ","]

or a mixture thereof. In another embodiment, A is

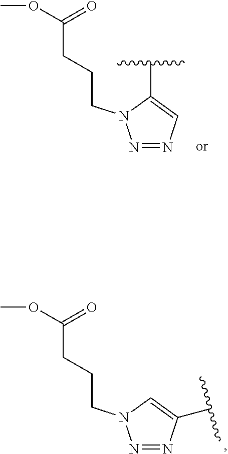 [SMILES: *c1cnnn1CCCC(=O)OC ; "or" ; *c1cn(CCCC(=O)OC)nn1 ; ","]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is

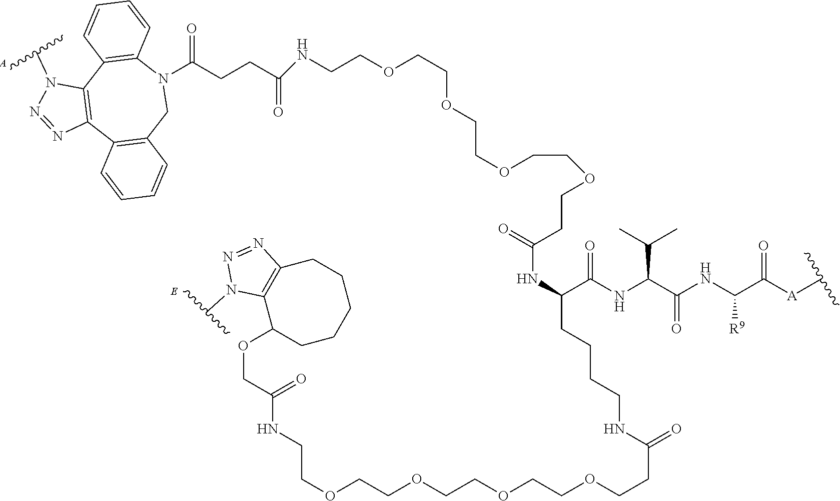

or a pharmaceutically acceptable salt, solvate, or stereoisomeric form thereof, or a regioisomer thereof, or mixture of regioisomers thereof, wherein
each

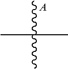

is a bond to a transglutaminse-modified binding agent;
each

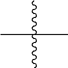

is a bond to the payload;
each

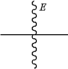

is a bond to the enhancement group;
each R⁹ is —CH₃ or —(CH₂)₃N(H)C(O)NH₂; and
each A is —O—, —N(H)—,

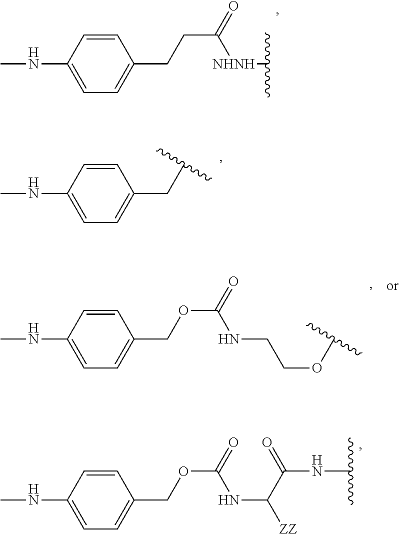

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

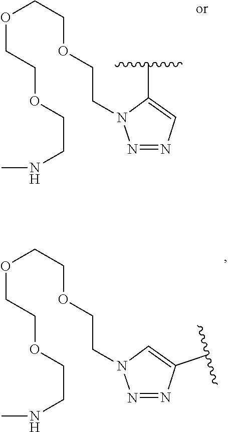 [SMILES: *c1cnnn1CCOCCOCCOCCNC ; *c1cn(CCOCCOCCOCCNC)nn1]

or a mixture thereof. Alternatively, in another embodiment, A is

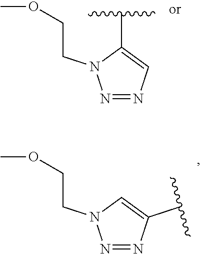

or a mixture thereof. In another embodiment, A is

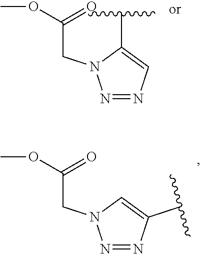 [SMILES: *c1cnnn1CC(=O)OC ; *c1cn(CC(=O)OC)nn1]

or a mixture thereof. In another embodiment, A is

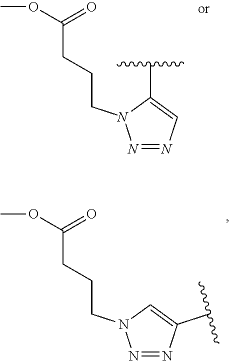 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N(CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is one, two, three, four, or five, and m is one, two, three, four, or five. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is one, two, three, four, or five. In another embodiment, the alkyl, heteroalkyl, alkylenyl, or heteroalkylenyl sulfonic acid is —(CH₂)ₙ—C(O)NH—(CH₂)₁₋₅SO₃H, wherein n is one, two, three, four, or five. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is one, two, three, four, or five. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is one, two, three, four, or five.

In some embodiments, the linker is

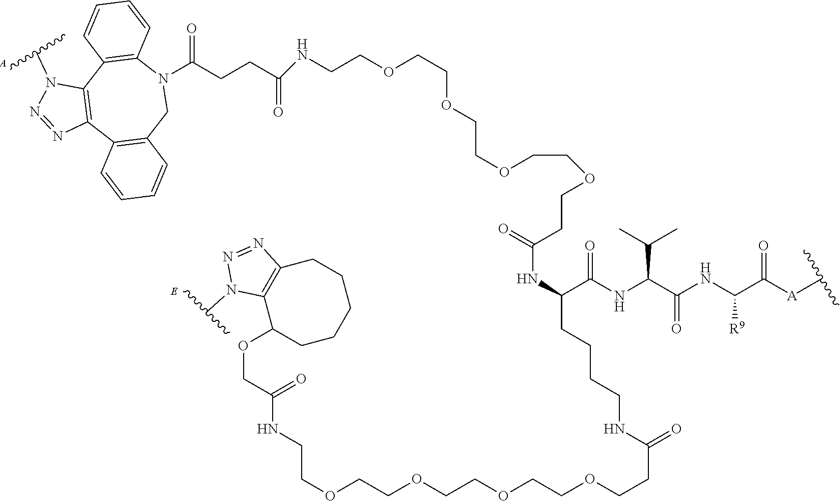

or a pharmaceutically acceptable salt, solvate, or stereoisomeric form thereof, or a regioisomer thereof, or mixture of regioisomers thereof, wherein
each

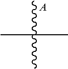

is a bond to a transglutaminase-modified binding agent;
each

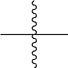

is a bond to the payload;
each R⁹ is —CH₃ or —(CH₂)₃N(H)C(O)NH₂; and
each A is —O—, —N(H)—,

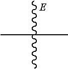

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

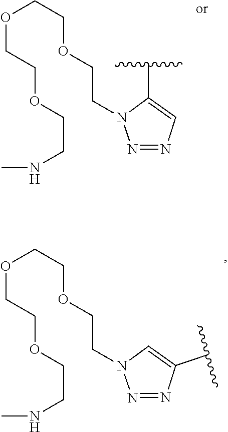 [SMILES: *c1cnnn1CCOCCOCCOCCNC ; *c1cn(CCOCCOCCOCCNC)nn1]

or a mixture thereof. Alternatively, in another embodiment, A is

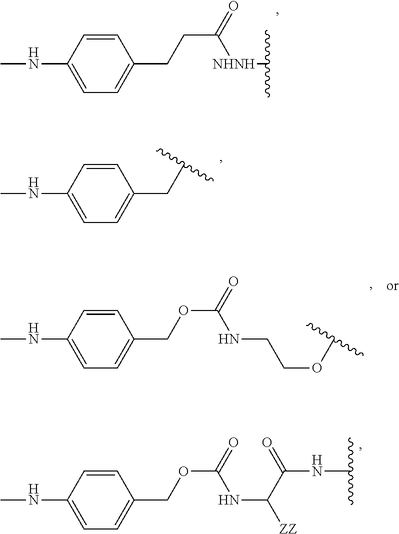

or a mixture thereof. In another embodiment, A is

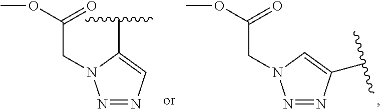 [SMILES: *c1cnnn1CC(=O)OC ; *c1cn(CC(=O)OC)nn1]

or a mixture thereof. In another embodiment, A is

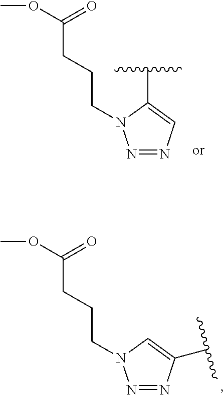 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent. In certain embodiments, the enhancement agent is a hydrophilic group. In certain embodiments, the enhancement agent is cyclodextrin. In certain embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In certain embodiments, sugars include, without limitation, monosaccharides, disaccharides, and polysaccharides. Exemplary monosaccharides include glucose, ribose, deoxyribose, xylose, arabinose, mannose, galactose, fructose, and the like. In certain embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In certain embodiments, the cyclodextrin is alpha cyclodextrin, beta cyclodextrin, or gamma cyclodextrin, or mixtures thereof. In certain embodiments, the cyclodextrin is alpha cyclodextrin. In certain embodiments, the cyclodextrin is beta cyclodextrin. In certain embodiments, the cyclodextrin is gamma cyclodextrin. In certain embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N(CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is one, two, three, four, or five, and m is one, two, three, four, or five. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is one, two, three, four, or five. In another embodiment, the alkyl, heteroalkyl, alkylenyl, or heteroalkylenyl sulfonic acid is —(CH₂)ₙ—C(O)NH—(CH₂)₁₋₅SO₃H, wherein n is one, two, three, four, or five. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is one, two, three, four, or five. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is one, two, three, four, or five. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is one, two, three, four or five.

In some embodiments, the linker is

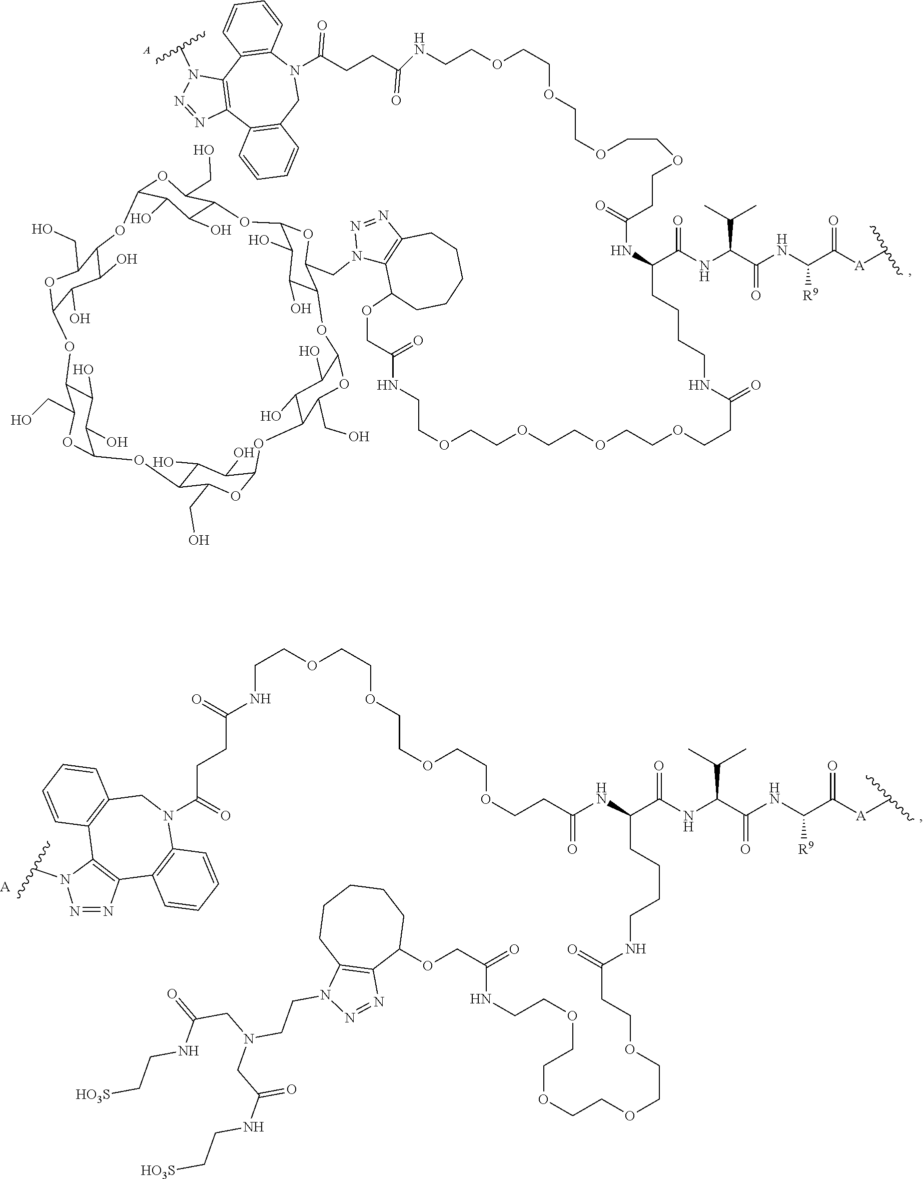

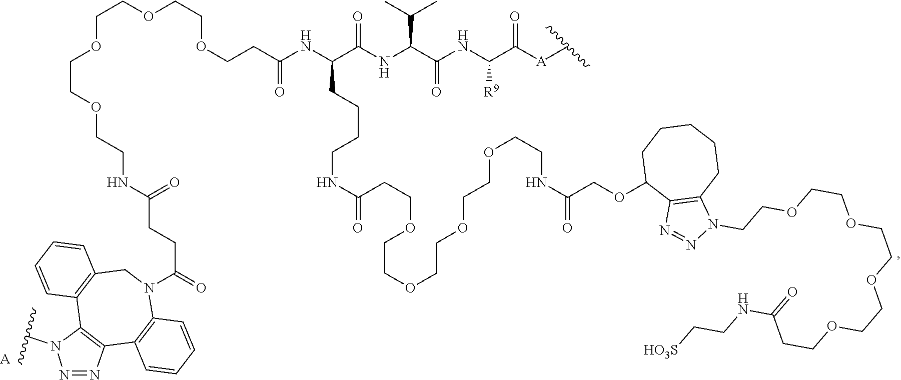

or a pharmaceutically acceptable salt, solvate, or stereoisomeric form thereof, or a regioisomer thereof, or mixture of regioisomers thereof, wherein
each

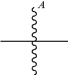

is a bond to a transglutaminanse-modified binding agent;
each

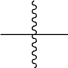

is a bond to the payload;
R⁹ is —CH₃ or —(CH₂)₃N(H)C(O)NH₂; and
A is —O—, —N(H)—,

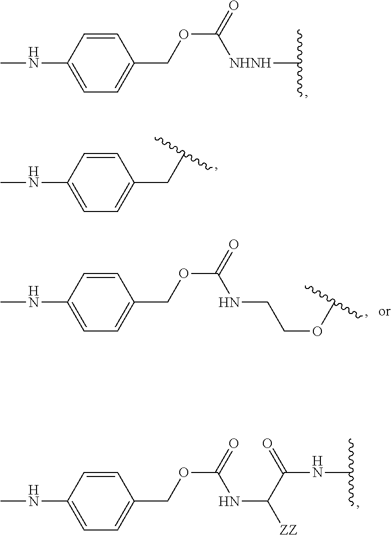

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

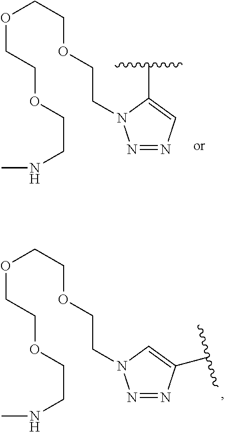 [SMILES: *c1cnnn1CCOCCOCCOCCNC ; *c1cn(CCOCCOCCOCCNC)nn1]

or a mixture thereof. Alternatively, in another embodiment, A is

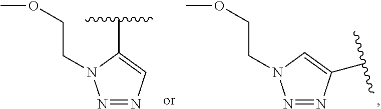

or a mixture thereof. In another embodiment, A is

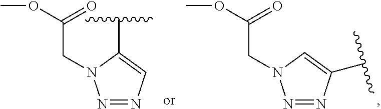 [SMILES: *c1cnnn1CC(=O)OC ; *c1cn(CC(=O)OC)nn1]

or a mixture thereof. In another embodiment, A is

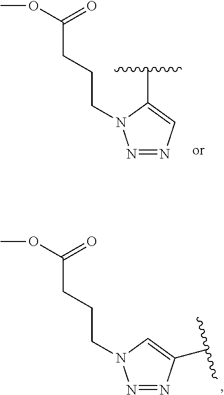 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In some embodiments, the linker is

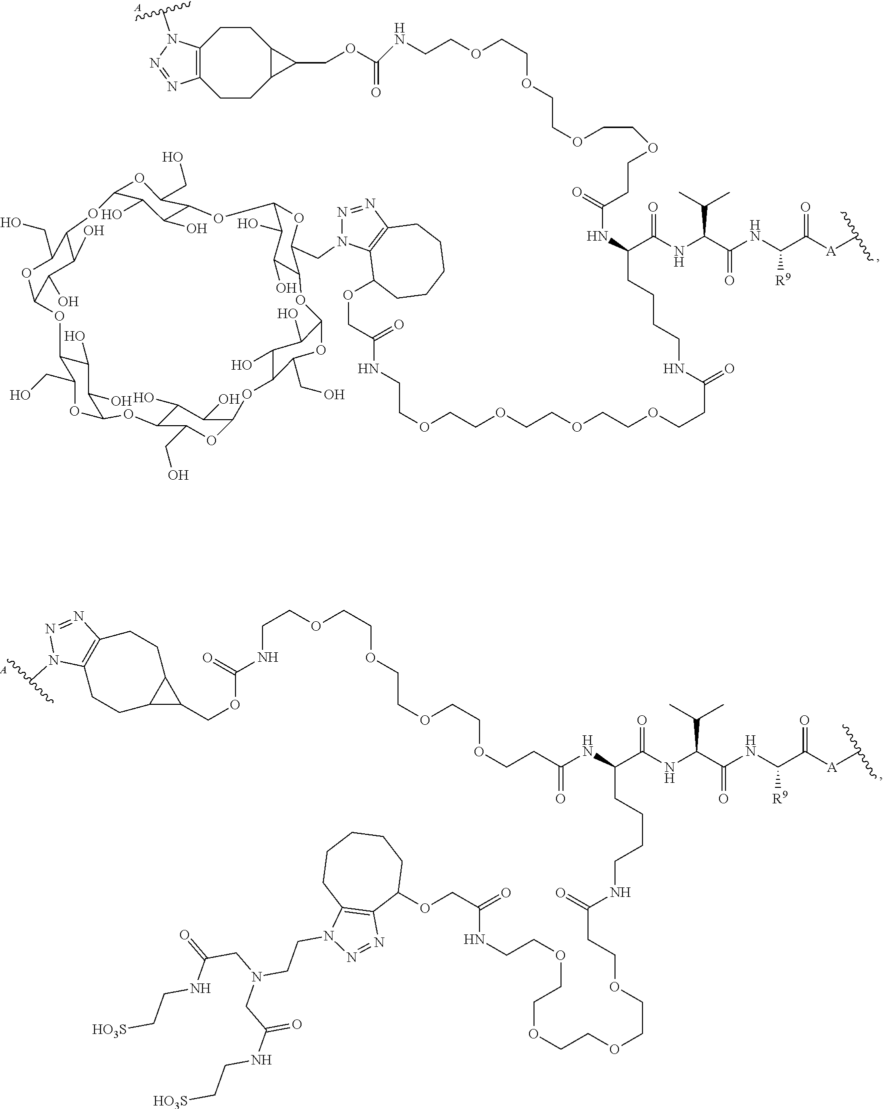

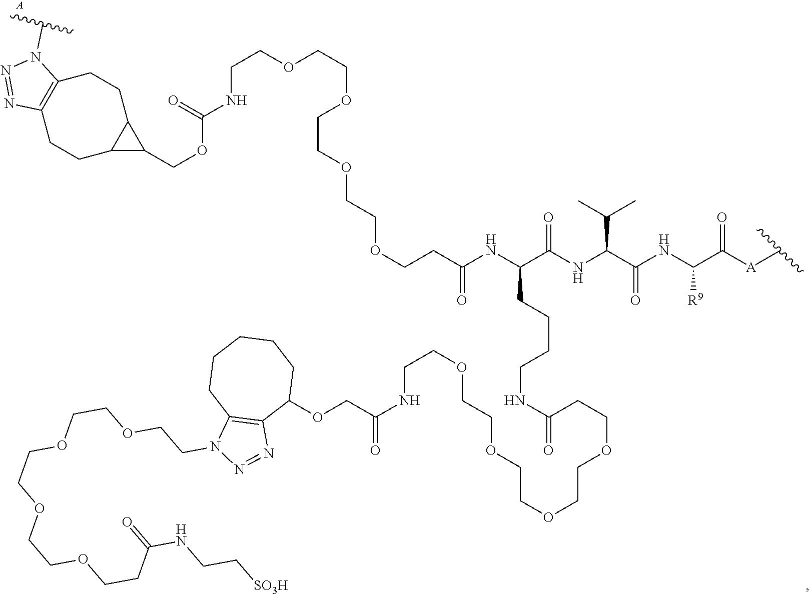

or a pharmaceutically acceptable salt, solvate, or stereoisomeric form thereof, or a regioisomer thereof, or mixture of regioisomers thereof, wherein
each

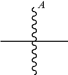

is a bond to a transglutaminase-modified binding agent;
each

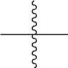

is a bond to the payload;
R⁹ is —CH₃ or —(CH₂)₃N(H)C(O)NH₂; and
A is —O—, —N(H)—,

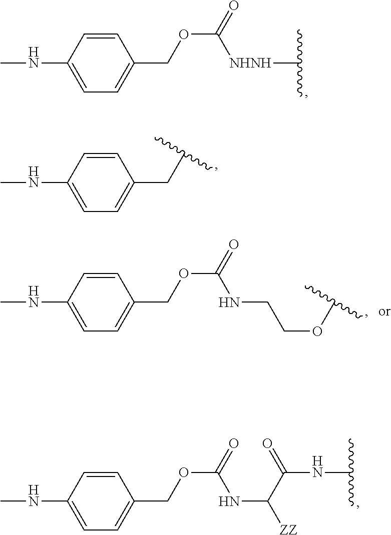

where ZZ is hydrogen, or a side chain for an amino acid as discussed elsewhere herein. For example, in one embodiment, ZZ is C₁₋₆ alkyl. By way of further example, in one embodiment, ZZ is C₁₋₆ heteroalkyl. In particular embodiments of this paragraph, A may be derived from a primary amine compound or a residue thereof where X is —N₃, as described elsewhere herein. In these embodiments, a 1,2,3-triazole residue is derived from the azide following participation in a click chemistry reaction, as described elsewhere herein, with an alkyne or terminal acetylene of a compound or payload described herein. Accordingly, in one non-limiting example, A is

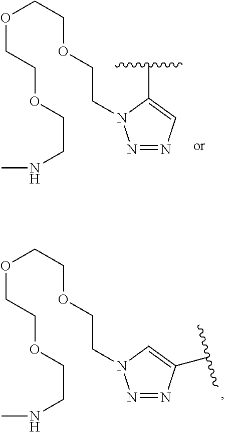 [SMILES: *c1cnnn1CCOCCOCCOCCNC ; *c1cn(CCOCCOCCOCCNC)nn1]

or a mixture thereof. Alternatively, in another embodiment, A is

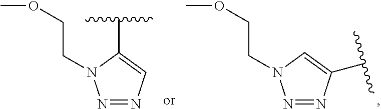

or a mixture thereof. In another embodiment, A is

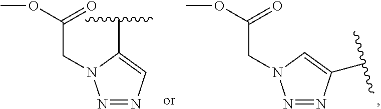 [SMILES: *c1cnnn1CC(=O)OC ; *c1cn(CC(=O)OC)nn1]

or a mixture thereof. In another embodiment, A is

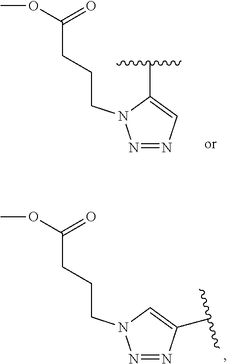 [SMILES: *c1cnnn1CCCC(=O)OC ; *c1cn(CCCC(=O)OC)nn1]

or a mixture thereof. As discussed above, the bond to the binding agent can be direct, or via a spacer. In certain embodiments, the bond to the binding agent is via a PEG spacer to a glutamine residue of the binding agent.

In particular embodiments, disclosed compounds, payloads, or prodrug payloads with an alkyne or terminal acetylene may be linked to a binding agent derivatized with —PEG-N₃ linked to a glutamine residue (viz., a transglutaminase-modified binding agent). Exemplary —N₃ derivatized binding agents (viz., transglutaminase-modified binding agents), methods for their preparation, and methods for their use are provided herein. In certain embodiments, a compound or payload with an alkyne described herein suitable for participation in 1,3-cycloadditions with binding agents derivatized with —PEG-N₃ provide regioisomeric 1,2,3-triazolyl linked moieties. For example, in certain embodiments, compounds or payloads linked to the binding agent may be

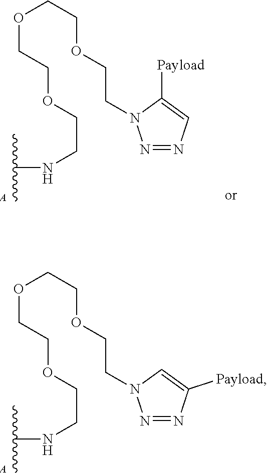

or a mixture thereof, where each

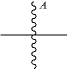

is a bond to the binding agent.

### Linker-Payloads

In certain embodiments, linker-payloads or linker-prodrug payloads (i.e., these descriptors are interchangeably used throughout) include any specific compound embraced by any one or more of Formulae I, Ia, Iaa, II, III, IV, V, or VI above, bonded to a linker, wherein the linker(s) described herein include a moiety that is reactive with an antibody or antigen binding fragment thereof described herein. In particular embodiments, the linker is bonded to a heterocycle comprising nitrogen, R¹, R₂, R³, R⁶, or R⁷ in any one or more of Formulae I, Ia, Iaa, II, III, IV, V, or VI above.

In one embodiment, the linker-payload has a Formula LPa, LPb, LPc, LPd, or LPe

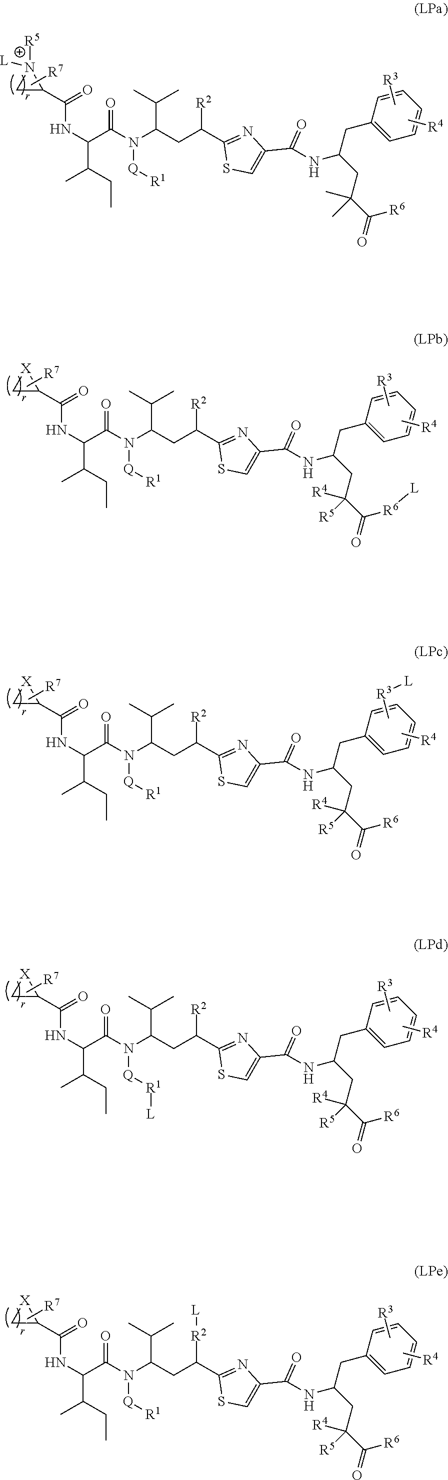

wherein L is a linker.

In certain embodiments, R² is —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH with a covalent bond to L from a terminal oxygen in any one of —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH; or R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂. In one embodiment, R² is —O—C(O)—NH—CH₂—CH(OH)—CH₂OH with a covalent bond to L from the terminal oxygen in —O—C(O)—NH—CH₂—CH(OH)—CH₂OH. In one embodiment, R² is —O—(CH₂)₃—OH with a covalent bond to L from the terminal oxygen in —O—(CH₂)₃—OH. In one embodiment, R² is —O—C(O)—NH—(CH₂)₂—OH with a covalent bond to L from the terminal oxygen in —O—C(O)—NH—(CH₂)₂—OH. In one embodiment, R² is —O—C(O)—NH—(CH₂CH₂O)₂—(CH₂)₂OH with a covalent bond to L from the terminal oxygen in —O—C(O)—NH—(CH₂CH₂O)₂—(CH₂)₂OH. In one embodiment, R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂.

In certain embodiments, R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, or —NH—CH₂—(CH₂O)₂—(CH₂)₂—NH₂. In certain embodiments, R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂. In one embodiment, R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂). In one embodiment, R³ is —OH with a covalent bond to L from the oxygen in —OH. In one embodiment, R³ is —NH—(CH₂)₂OH with a covalent bond to L from the terminal oxygen in —NH—(CH₂)₂OH. In one embodiment, R³ is —NH—CH₂—C(O)—OH with a covalent bond to L from the terminal oxygen in —NH—CH₂—C(O)—OH. In one embodiment, R³ is —N(CH₂CH₂OH)(C(O)CH₂NH₂) with a covalent bond to L from the terminal oxygen in —N(CH₂CH₂OH)(C(O)CH₂NH₂). In one embodiment, R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂. In one embodiment, R³ is —NH₂ with a covalent bond to L from the nitrogen in —NH₂. In one embodiment, R³ is —NH—CH₂—C(O)—NH₂ with a covalent bond to L from the terminal nitrogen in any one of —NH—CH₂—C(O)—NH₂. In one embodiment, R³ is —NH—C(O)—CH₂NH₂ with a covalent bond to L from the terminal nitrogen in any one of —NH—C(O)—CH₂NH₂. In one embodiment, R³ is —NH—[(CH₂)₂OH]—C(O)—NH₂ with a covalent bond to L from the terminal nitrogen in —NH—[(CH₂)₂OH]—C(O)—NH₂. In one embodiment, R³ is —NH—CH₂—(CH₂O)₂—(CH₂)₂—NH₂ with a covalent bond to L from the terminal nitrogen in —NH—CH₂—(CH₂O)₂—(CH₂)₂—NH₂. In one embodiment, R³ is —N(CH₂CH₂OH)(C(O)CH₂NH₂) with a covalent bond to L from the terminal nitrogen in —N(CH₂CH₂OH)(C(O)CH₂NH₂). In one embodiment, R³ is —NH—CH₂—C(O)—NH—(CH₂CH₂O)₂—(CH₂)₂NH₂ with a covalent bond to L from the terminal nitrogen in —NH—CH₂—C(O)—NH—(CH₂CH₂O)₂—(CH₂)₂NH₂.

In certain embodiments, R⁵ is a covalent bond to L; or R⁵ is —(CH₂)₂—OH or —CH₂—C(O)—OH with a covalent bond to L from the terminal oxygen in —(CH₂)₂—OH or —CH₂—C(O)—OH; or R⁵ is —(CH₂)₂—NH₂, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue with a covalent bond to L from the nitrogen in any one of —(CH₂)₂—NH₂, —(CH₂)₂—O—(CH₂)₂—NH₂, —(CH₂CH₂—O)₂—(CH₂)₂—NH₂, or —C(O)—CH₂—NH₂, the first N-terminal amino acid residue, or the first amino acid residue. In one embodiment, R⁵ is a covalent bond to L. In one embodiment, R⁵ is —(CH₂)₂—OH or —CH₂—C(O)—OH with a covalent bond to L from the terminal oxygen in —(CH₂)₂—OH or —CH₂—C(O)—OH. In one embodiment, R⁵ is —(CH₂)₂—OH with a covalent bond to L from the terminal oxygen in —(CH₂)₂—OH. In one embodiment, R⁵ is —CH₂—C(O)—OH with a covalent bond to L from the terminal oxygen in —CH₂—C(O)—OH. In one embodiment, R⁵ is —(CH₂)₂—NH₂, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue with a covalent bond to L from the nitrogen in any one of —(CH₂)₂—NH₂, —(CH₂)₂—O—(CH₂)₂—NH₂, —(CH₂CH₂—O)₂—(CH₂)₂—NH₂, or —C(O)—CH₂—NH₂, the first N-terminal amino acid residue, or the first amino acid residue. In one embodiment, R⁵ is —(CH₂)₂—NH₂ with a covalent bond to L from the nitrogen in —(CH₂)₂—NH₂. In one embodiment, R⁵ is —(CH₂)₂—O—(CH₂)₂—NH₂ with a covalent bond to L from the nitrogen in —(CH₂)₂—O—(CH₂)₂—NH₂. In one embodiment, R⁵ is —(CH₂CH₂—O)₂—(CH₂)₂—NH₂ with a covalent bond to L from the nitrogen in —(CH₂CH₂—O)₂—(CH₂)₂—NH₂. In one embodiment, R⁵ is —C(O)—CH₂—NH₂ with a covalent bond to L from the nitrogen in —C(O)—CH₂—NH₂. In one embodiment, R⁵ is a first N-terminal amino acid residue with a covalent bond to L from the nitrogen in the first N-terminal amino acid residue. In one embodiment, R⁵ is a first amino acid residue with a covalent bond to L from the nitrogen in the first amino acid residue.

In one embodiment, R⁶ is —OH, —NHCH₂C(O)OH, or —NH—C(O)OH with a covalent bond to L from the terminal oxygen in any one of —OH, —NHCH₂C(O)OH, or —NH—C(O)OH. In one embodiment, R⁶ is —OH with a covalent bond to L from the oxygen in —OH. In one embodiment, R⁶ is —NHCH₂C(O)OH with a covalent bond to L from the terminal oxygen in —NHCH₂C(O)OH. In one embodiment, R⁶ is —NH—C(O)OH with a covalent bond to L from the terminal oxygen in —NH—C(O)OH.

In one embodiment, the linker-payload has a structure of Formula LPa′

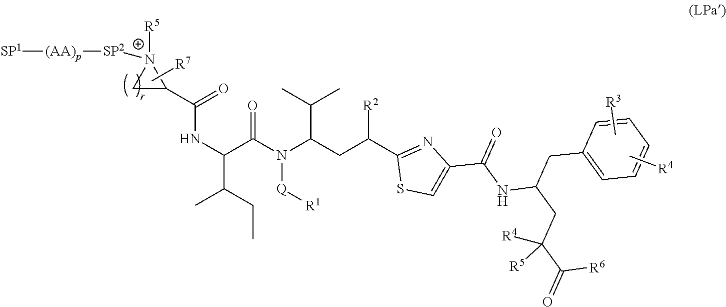

wherein SP¹, (AA)p, SP², Q, R¹, R₂, R³, R⁴, R⁵, R³, R⁷, and r are as described in any of the embodiments disclosed herein. In one embodiment, the linker-payload has a structure of Formula LPb′

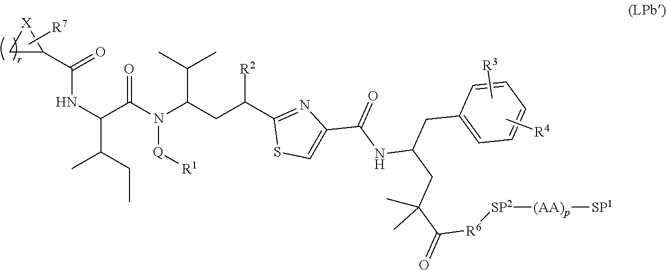

wherein SP¹, (AA)p, SP², Q, R¹, R², R³, R⁴, R⁵, R³, R⁷, and r are as described in any of the embodiments disclosed herein. In one embodiment, the linker-payload has a structure of Formula LPc′

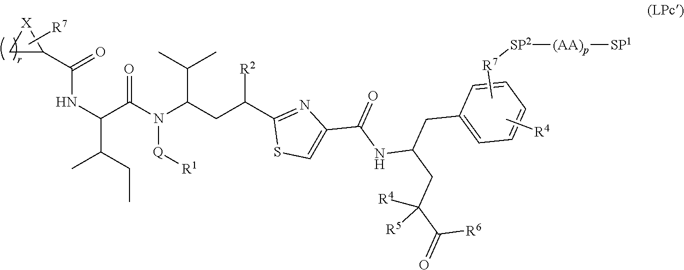

wherein SP′, (AA)p, SP², Q, R¹, R², R³, R⁴, R⁵, R³, R⁷, and r are as described in any of the embodiments disclosed herein. In one embodiment, the linker-payload has a structure of Formula LPd′

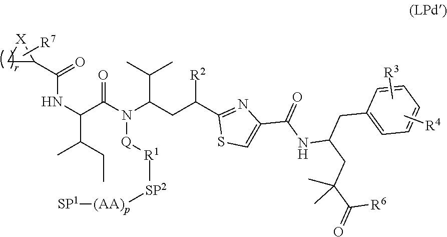

wherein SP′, (AA)p, SP², Q, R¹, R², R³, R⁴, R⁵, R³, R⁷, and r are as described in any of the embodiments disclosed herein. In one embodiment, the linker-payload has a structure of Formula LPe′

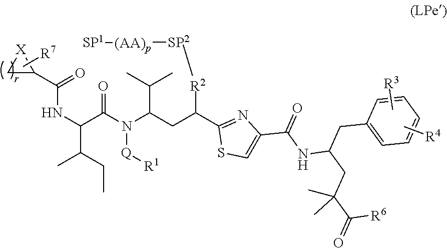

wherein SP′, (AA)p, SP², Q, R¹, R², R³, R⁴, R⁵, R³, R⁷, and r are as described in any of the embodiments disclosed herein. In any of the embodiments in this paragraph, Formulae LPa′, LPb′, LPc′, LPd′, or LPe′ may be a pharmaceutically acceptable salt or prodrug thereof. In any of the embodiments in this paragraph, p is zero, one, two, three, four, five, six, seven, eight, nine, or ten. In any of the embodiments in this paragraph, p is zero. In any of the embodiments in this paragraph, p is one. In any of the embodiments in this paragraph, p is two. In any of the embodiments in this paragraph, p is three. In any of the embodiments in this paragraph, p is four. In any of the embodiments in this paragraph, p is five. In any of the embodiments in this paragraph, p is six. In any of the embodiments in this paragraph, p is seven. In any of the embodiments in this paragraph, p is eight. In any of the embodiments in this paragraph, p is nine. In any of the embodiments in this paragraph, p is ten. In one embodiment, the linker-payload has a structure of LPa′, LPb′, LPc′, LPd′, or LPe′, wherein the —SP²— spacer, when present, is

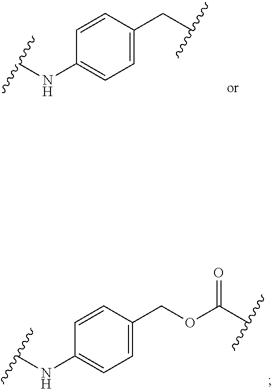 [SMILES: *Cc1ccc(N*)cc1 ; *Nc1ccc(COC(*)=O)cc1]

the second -(AA)ₚ- is

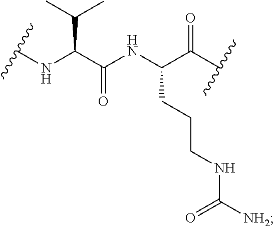 [SMILES: *N[C@H](C(=O)N[C@@H](CCCNC(N)=O)C(*)=O)C(C)C]

the —SP¹— spacer is

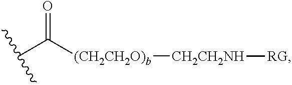

wherein RG is a reactive group; and b is an integer from one to four. In one embodiment, the linker-payload has a structure of LPa′, LPb′, LPc′, LPd′, or LPe′, wherein Q is —O—. In one embodiment, the linker-payload has a structure of LPa′, LPb′, LPc′, LPd′, or LPe′, wherein Q is —CH₂—; X is —NR⁵; R⁵ is —CH₃ or —(CH₂)₂—OH; R¹ is —C₅ alkyl; R⁶ is —OH; R⁷ when present is —CH₃; and r is three. In one embodiment, the linker-payload has a structure of LPa′, LPb′, LPc′, LPd′, or LPe′, wherein Q is —CH₂—; X is —NR 5; R⁵ is —CH₃ or —(CH₂)₂—OH; R¹ is —C₅ alkyl; R⁶ is —OH; R⁷ when present is —CH₃; and r is four. In one embodiment, the linker-payload has a structure of LPc′, or a pharmaceutically acceptable salt thereof. In one embodiment, the linker-payload has a structure of LPc′, or a pharmaceutically acceptable salt thereof, wherein R³ is —NH—(CH₂)₂OH, —NH—CH₂—C(O)—NH₂, —NH—C(O)—CH₂NH₂, or —NH—CH₂—C(O)—NH—(CH₂CH₂O)₂—(CH₂)₂NH₂; or R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or R³ is —NH₂, —NH—CH₂—C(O)—NH₂, —NH—C(O)—CH₂NH₂, —NH—[(CH₂)₂O]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂O]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂; and R⁴ is hydrogen or —F. In one embodiment, the linker-payload has a structure of LPc′, or a pharmaceutically acceptable salt thereof, wherein R³ is —NH—(CH₂)₂OH. In one embodiment, the linker-payload has a structure of LPc′, or a pharmaceutically acceptable salt thereof, wherein R³ is —NH—CH₂—C(O)—NH₂. In one embodiment, the linker-payload has a structure of LPc′, or a pharmaceutically acceptable salt thereof, wherein R³ is —NH—C(O)—CH₂NH₂. In one embodiment, the linker-payload has a structure of LPc′, or a pharmaceutically acceptable salt thereof, wherein R³ is, —NH—CH₂—C(O)—NH—(CH₂CH₂O)₂—(CH₂)₂NH₂. In certain embodiments, R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂. In one embodiment, R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂). In one embodiment, R³ is —OH with a covalent bond to L from the oxygen in —OH. In one embodiment, R³ is —NH—(CH₂)₂OH with a covalent bond to L from the terminal oxygen in —NH—(CH₂)₂OH. In one embodiment, R³ is —NH—CH₂—C(O)—OH with a covalent bond to L from the terminal oxygen in —NH—CH₂—C(O)—OH. In one embodiment, R³ is —N(CH₂CH₂OH)(C(O)CH₂NH₂) with a covalent bond to L from the terminal oxygen in —N(CH₂CH₂OH)(C(O)CH₂NH₂). In one embodiment, R³ is —NH₂, —NH—CH₂—C(O)—NH₂, —NH—C(O)—CH₂NH₂, —NH—[(CH₂)₂O]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂O]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂. In one embodiment, R³ is —NH₂ with a covalent bond to L from the nitrogen in —NH₂. In one embodiment, R³ is —NH—CH₂—C(O)—NH₂ with a covalent bond to L from the terminal nitrogen in any one of —NH—CH₂—C(O)—NH₂. In one embodiment, R³ is —NH—C(O)—CH₂NH₂ with a covalent bond to L from the terminal nitrogen in any one of —NH—C(O)—CH₂NH₂. In one embodiment, R³ is —NH—[(CH₂)₂O]—C(O)—NH₂ with a covalent bond to L from the terminal nitrogen in —NH—[(CH₂)₂O]—C(O)—NH₂. In one embodiment, R³ is —NH—CH₂—(CH₂O)₂—(CH₂)₂—NH₂ with a covalent bond to L from the terminal nitrogen in —NH—CH₂—(CH₂O)₂—(CH₂)₂—NH₂. In one embodiment, R³ is —N(CH₂CH₂OH)(C(O)CH₂NH₂) with a covalent bond to L from the terminal nitrogen in —N(CH₂CH₂OH)(C(O)CH₂NH₂). In one embodiment, R³ is —NH—CH₂—C(O)—NH—(CH₂CH₂O)₂—(CH₂)₂NH₂ with a covalent bond to L from the terminal nitrogen in —NH—CH₂—C(O)—NH—(CH₂CH₂O)₂—(CH₂)₂NH₂. In one embodiment, the linker-payload has a structure of LPe′, or a pharmaceutically acceptable salt thereof. In one embodiment, the linker-payload has a structure of LPe′, or a pharmaceutically acceptable salt thereof, wherein R² is —O—C(O)—NH—(CH₂CH₂O)₂—(CH₂)₂OH. In one embodiment, the linker-payload has a structure of LPe′, or a pharmaceutically acceptable salt thereof, wherein R² is —O—C(O)—NH—(CH₂CH₂O)₂—(CH₂)₂O—. In one embodiment, the linker-payload has a structure of LPa′, LPb′, LPc′, LPd′, or LPe′, wherein Q is —O—. In one embodiment, the linker-payload has a structure of LPa′, LPb′, LPc′, LPd′, or LPe′, wherein Q is —CH₂—; X is —NR⁵; R¹ is —C₅ alkyl; R⁶ is —OH; R⁷ when present is —CH₃; and r is three. In one embodiment, the linker-payload has a structure of LPa′, or a pharmaceutically acceptable salt thereof. In one embodiment, the linker-payload has a structure of LPe′, or a pharmaceutically acceptable salt thereof, wherein R⁵ is —C(O)—CH₂—NH₂. In one embodiment, the linker-payload has a structure of LPe′, or a pharmaceutically acceptable salt thereof, wherein R⁵ is —C(O)—CH₂—NH—. In one embodiment, the linker-payload is selected from the group consisting of

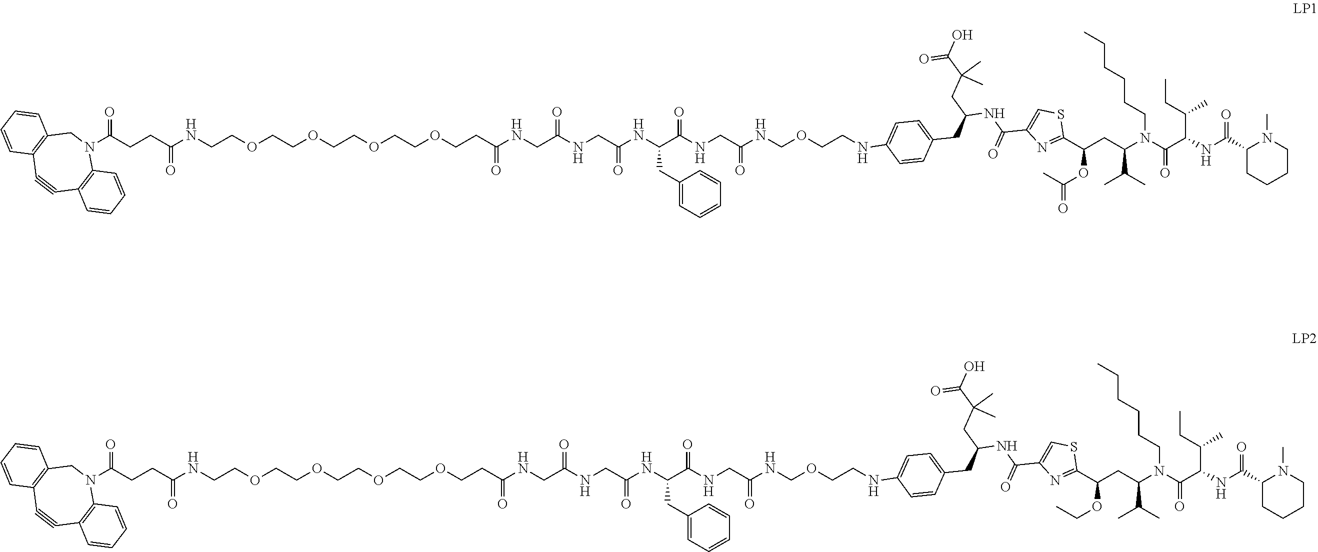 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCOCNC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C ; CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCOCNC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C]

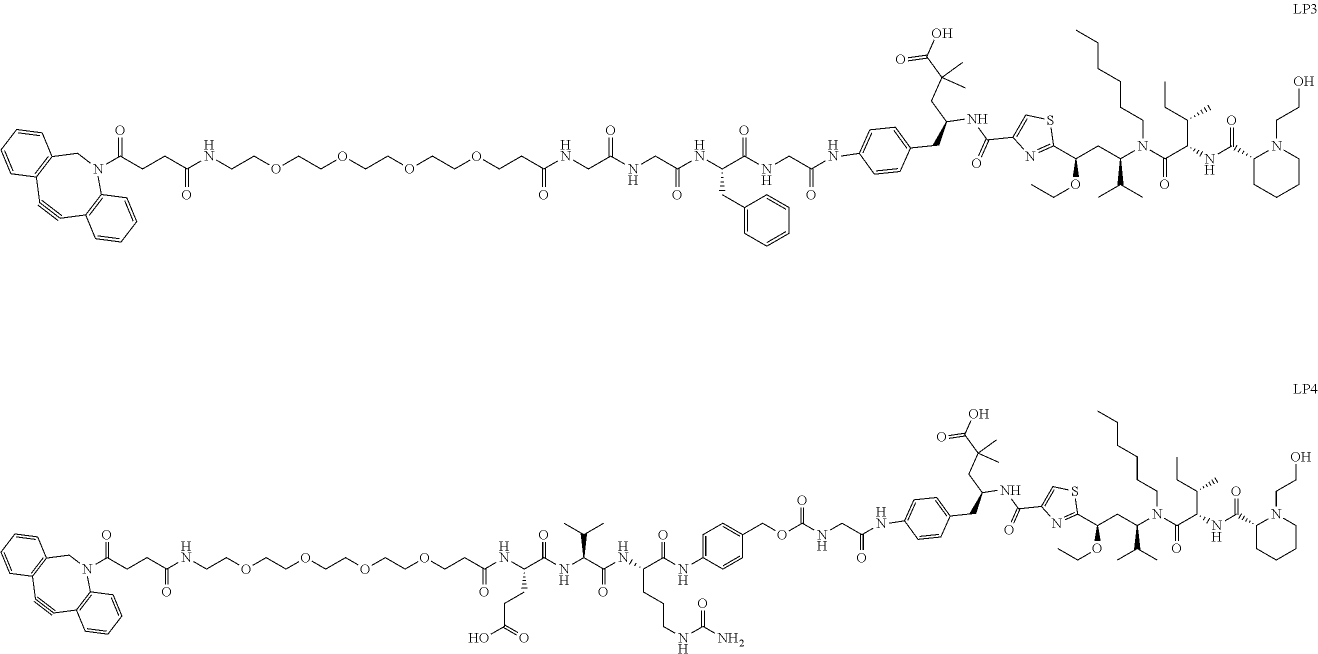 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C ; CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CNC(=O)OCc3ccc(NC(=O)[C@H](CCCNC(N)=O)NC(=O)[C@@H](NC(=O)[C@H](CCC(=O)O)NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C]

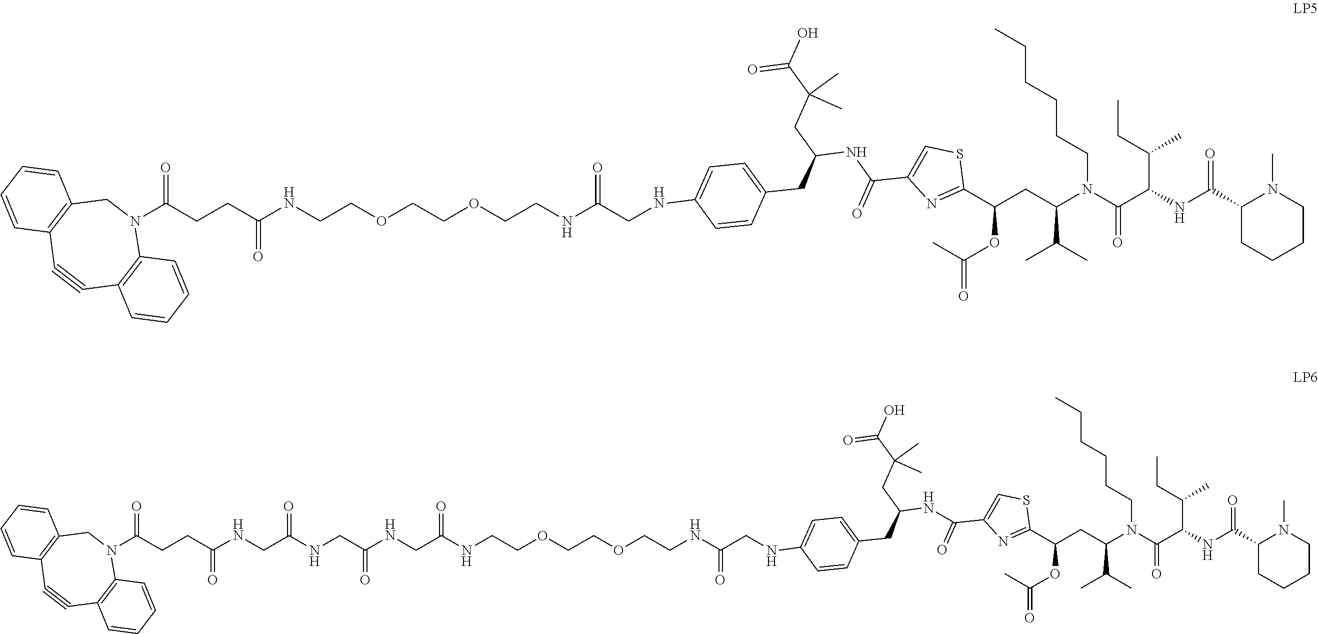 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCNC(=O)CCC(=O)N3Cc4ccccc4C#Cc4ccccc43)cc2)CC(C)(C)C(=O)O)cs1)C(C)C ; CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCNC(=O)CNC(=O)CNC(=O)CNC(=O)CCC(=O)N3Cc4ccccc4C#Cc4ccccc43)cc2)CC(C)(C)C(=O)O)cs1)C(C)C]

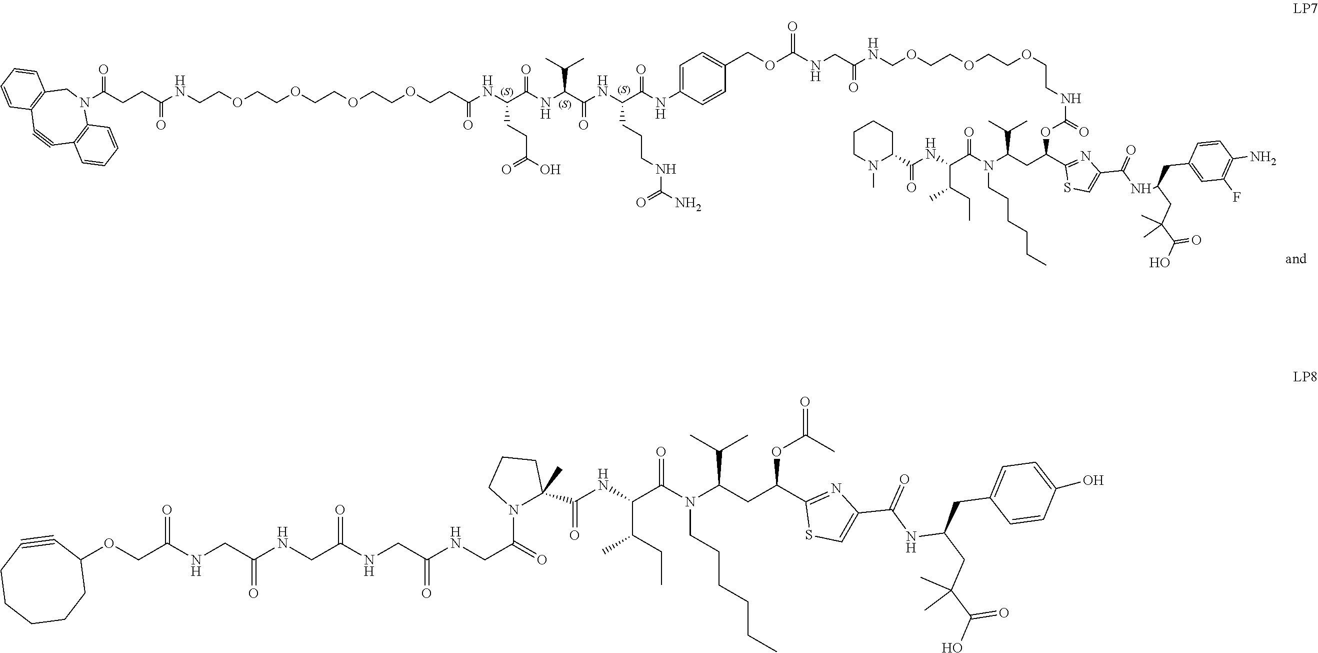

or
a pharmaceutically acceptable salt thereof. In one embodiment, the linker-payload selected from the group consisting of

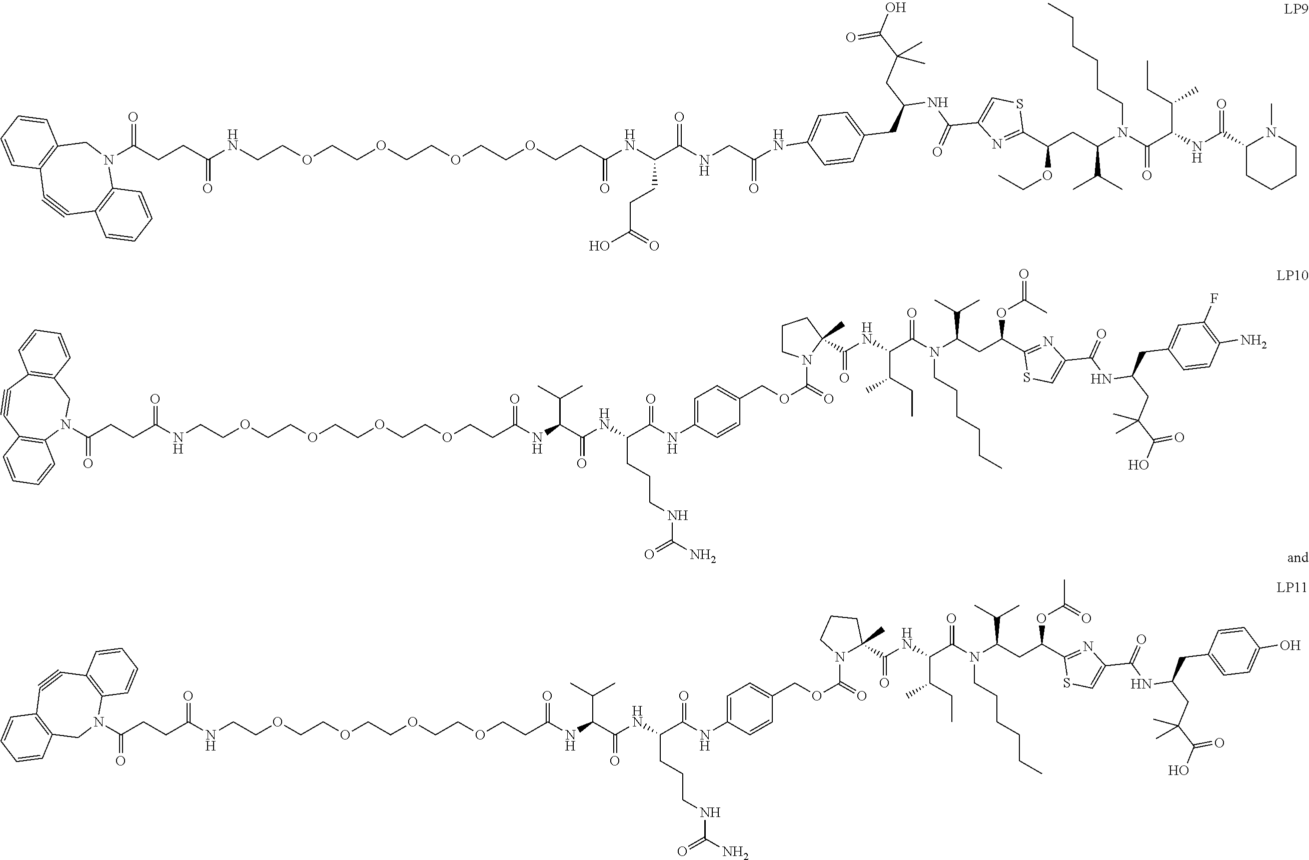

or
a pharmaceutically acceptable salt thereof.

### Conjugates/Antibody Drug Conjugates (ADCs)

Provided herein are antibodies or an antigen binding fragments thereof, wherein said antibody is conjugated to one or more compounds of Formula I, Ia, Iaa, II, III, IV, V, or VI as described herein.

Provided herein are conjugates having a Formula A, B, C, D, or E

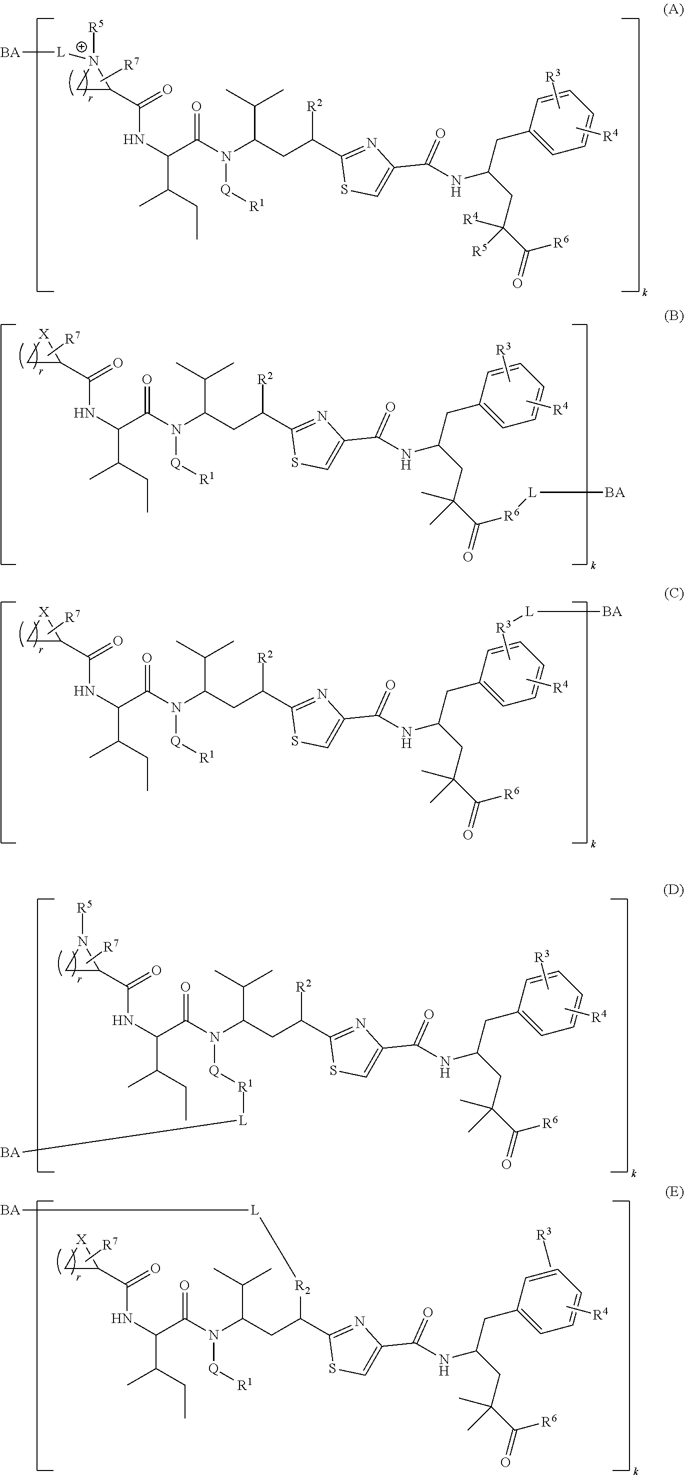

wherein L is a linker. In certain embodiments, Q, X, IV, R₂, R³, R⁴, R⁵, R⁶, R⁷, and r are as described above in the context of Formula I, and k is one, two, three, four, five, six, seven, eight, nine, or ten.

Provided herein are conjugates of Formula

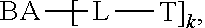

A, B, C, D, or E, wherein T is described elsewhere herein, or a pharmaceutically acceptable salt, solvate, regioisomeric, or stereoisomeric form thereof. In certain embodiments, R² is —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH with a covalent bond to L from a terminal oxygen in any one of —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH; or R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂. In one embodiment, R² is —O—C(O)—NH—CH₂—CH(OH)—CH₂OH with a covalent bond to L from the terminal oxygen in —O—C(O)—NH—CH₂—CH(OH)—CH₂OH. In one embodiment, R² is —O—(CH₂)₃—OH with a covalent bond to L from the terminal oxygen in —O—(CH₂)₃—OH. In one embodiment, R² is —O—C(O)—NH—(CH₂)₂—OH with a covalent bond to L from the terminal oxygen in —O—C(O)—NH—(CH₂)₂—OH. In one embodiment, R² is —O—C(O)—NH—(CH₂CH₂O)₂—(CH₂)₂OH with a covalent bond to L from the terminal oxygen in —O—C(O)—NH—(CH₂CH₂O)₂—(CH₂)₂OH. In one embodiment, R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂. In certain embodiments, R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, or —NH—CH₂—C(O)—NH—(CH₂CH₂O)₂—(CH₂)₂NH₂. In certain embodiments, R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂. In one embodiment, R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂). In one embodiment, R³ is —OH with a covalent bond to L from the oxygen in —OH. In one embodiment, R³ is —NH—(CH₂)₂OH with a covalent bond to L from the terminal oxygen in —NH—(CH₂)₂OH. In one embodiment, R³ is —NH—CH₂—C(O)—OH with a covalent bond to L from the terminal oxygen in —NH—CH₂—C(O)—OH. In one embodiment, R³ is —N(CH₂CH₂OH)(C(O)CH₂NH₂) with a covalent bond to L from the terminal oxygen in —N(CH₂CH₂OH)(C(O)CH₂NH₂). In one embodiment, R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂. In one embodiment, R³ is —NH₂ with a covalent bond to L from the nitrogen in —NH₂. In one embodiment, R³ is —NH—CH₂—C(O)—NH₂ with a covalent bond to L from the terminal nitrogen in any one of —NH—CH₂—C(O)—NH₂. In one embodiment, R³ is —NH—C(O)—CH₂NH₂ with a covalent bond to L from the terminal nitrogen in any one of —NH—C(O)—CH₂NH₂. In one embodiment, R³ is —NH—[(CH₂)₂OH]—C(O)—NH₂ with a covalent bond to L from the terminal nitrogen in —NH—[(CH₂)₂OH]—C(O)—NH₂. In one embodiment, R³ is —NH—CH₂—(CH₂O)₂—(CH₂)₂—NH₂ with a covalent bond to L from the terminal nitrogen in —NH—CH₂—(CH₂O)₂—(CH₂)₂—NH₂. In one embodiment, R³ is —N(CH₂CH₂OH)(C(O)CH₂NH₂) with a covalent bond to L from the terminal nitrogen in —N(CH₂CH₂OH)(C(O)CH₂NH₂). In one embodiment, R³ is —NH—CH₂—C(O)—NH—(CH₂CH₂O)₂—(CH₂)₂NH₂ with a covalent bond to L from the terminal nitrogen in —NH—CH₂—C(O)—NH—(CH₂CH₂O)₂—(CH₂)₂NH₂.

In certain embodiments, R⁵ is a covalent bond to L; or R⁵ is —(CH₂)₂—OH or —CH₂—C(O)—OH with a covalent bond to L from the terminal oxygen in —(CH₂)₂—OH or —CH₂—C(O)—OH; or R⁵ is —(CH₂)₂—NH₂, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue with a covalent bond to L from the nitrogen in any one of —(CH₂)₂—NH₂, —(CH₂)₂—O—(CH₂)₂—NH₂, —(CH₂CH₂—O)₂—(CH₂)₂—NH₂, or —C(O)—CH₂—NH₂, the first N-terminal amino acid residue, or the first amino acid residue. In one embodiment, R⁵ is a covalent bond to L. In one embodiment, R⁵ is —(CH₂)₂—OH or —CH₂—C(O)—OH with a covalent bond to L from the terminal oxygen in —(CH₂)₂—OH or —CH₂—C(O)—OH. In one embodiment, R⁵ is —(CH₂)₂—OH with a covalent bond to L from the terminal oxygen in —(CH₂)₂—OH. In one embodiment, R⁵ is —CH₂—C(O)—OH with a covalent bond to L from the terminal oxygen in —CH₂—C(O)—OH. In one embodiment, R⁵ is —(CH₂)₂—NH₂, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue with a covalent bond to L from the nitrogen in any one of —(CH₂)₂—NH₂, —(CH₂)₂—O—(CH₂)₂—NH₂, —(CH₂CH₂—O)₂—(CH₂)₂—NH₂, or —C(O)—CH₂—NH₂, the first N-terminal amino acid residue, or the first amino acid residue. In one embodiment, R⁵ is —(CH₂)₂—NH₂ with a covalent bond to L from the nitrogen in —(CH₂)₂—NH₂. In one embodiment, R⁵ is —(CH₂)₂—O—(CH₂)₂—NH₂ with a covalent bond to L from the nitrogen in —(CH₂)₂—O—(CH₂)₂—NH₂. In one embodiment, R⁵ is —(CH₂CH₂—O)₂—(CH₂)₂—NH₂ with a covalent bond to L from the nitrogen in —(CH₂CH₂—O)₂—(CH₂)₂—NH₂. In one embodiment, R⁵ is —C(O)—CH₂—NH₂ with a covalent bond to L from the nitrogen in —C(O)—CH₂—NH₂. In one embodiment, R⁵ is a first N-terminal amino acid residue with a covalent bond to L from the nitrogen in the first N-terminal amino acid residue. In one embodiment, R⁵ is a first amino acid residue with a covalent bond to L from the nitrogen in the first amino acid residue.

In one embodiment, R⁶ is —OH, —NHCH₂C(O)OH, or —NH—C(O)OH with a covalent bond to L from the terminal oxygen in any one of —OH, —NHCH₂C(O)OH, or —NH—C(O)OH. In one embodiment, R⁶ is —OH with a covalent bond to L from the oxygen in —OH. In one embodiment, R⁶ is —NHCH₂C(O)OH with a covalent bond to L from the terminal oxygen in —NHCH₂C(O)OH. In one embodiment, R⁶ is —NH—C(O)OH with a covalent bond to L from the terminal oxygen in —NH—C(O)OH.

In certain embodiments, Q, X, R¹, R², R³, R⁴, R⁵, R⁶, R⁷, and r are as described above in the context of Formula I, Ia, Iaa, II, III, IV, V, or VI as described herein, and k is one, two, three, four, five, six, seven, eight, nine, or ten. In certain embodiments, k is a range from 1-2, 1-3, 2-3, 2-4, 3-4, or 1-4.

Provided herein are conjugates of A′, B′, C′, D′, or E′

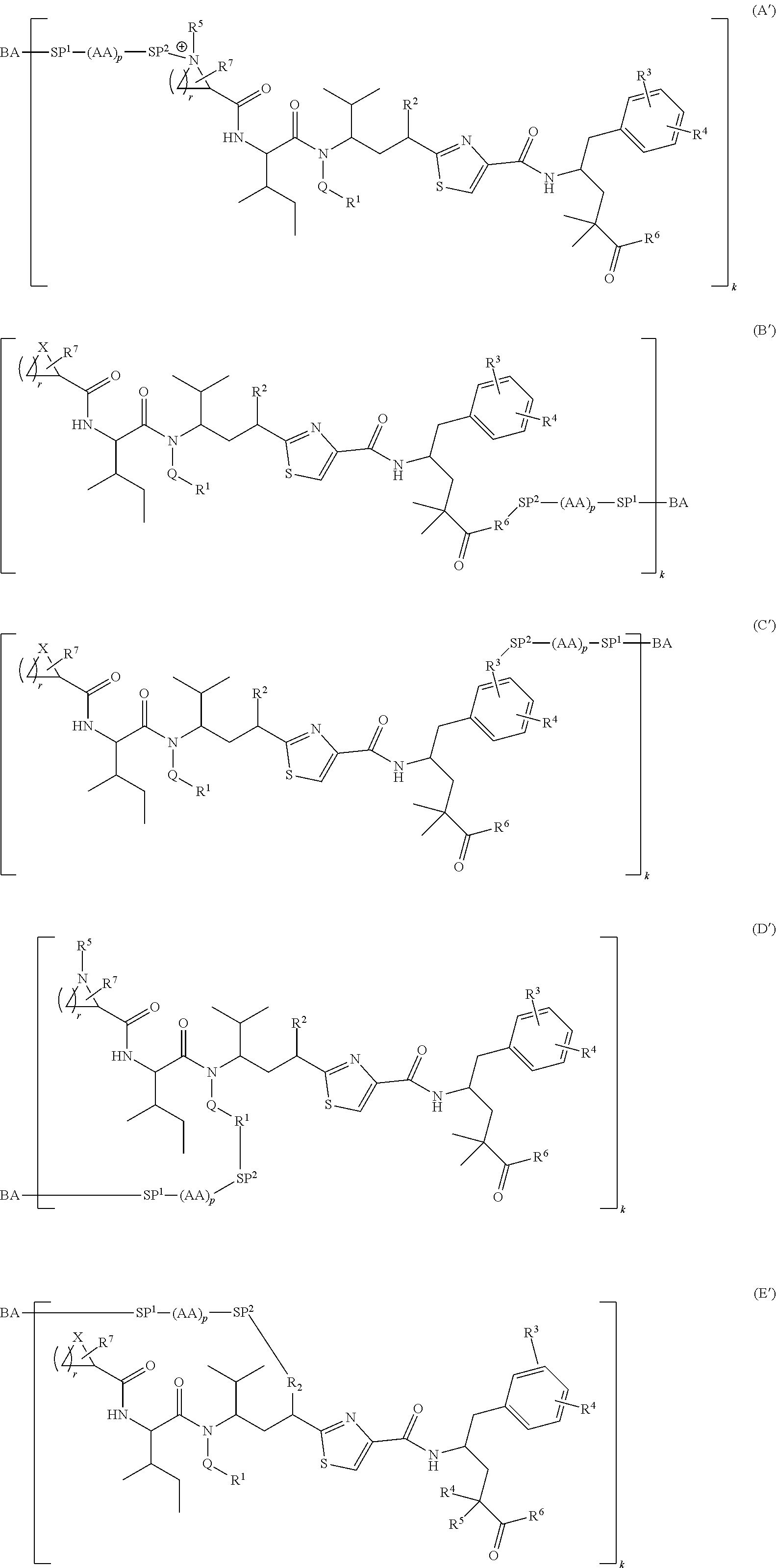

or a pharmaceutically acceptable salt, prodrug, solvate, regioisomeric, or stereoisomeric form thereof, wherein SP¹ and SP², when present, are spacer groups; each AA, when present, is a second amino acid residue; and p is an integer from zero to ten. In certain embodiments, Q, X, R₂, R³, R⁴, R⁵, R⁶, R⁷, and r are as described above in the context of Formula I, Ia, Iaa, II, III, IV, V, or VI as described herein, and k is one, two, three, four, five, six, seven, eight, nine, or ten. In certain embodiments, k is a range from 1-2, 1-3, 2-3, 2-4, 3-4, or 1-4. In certain embodiments, the —SP²-spacer, when present, is

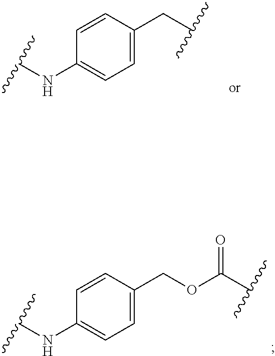 [SMILES: *Cc1ccc(N*)cc1 ; *Nc1ccc(COC(*)=O)cc1]

the second -(AA)ₚ- is

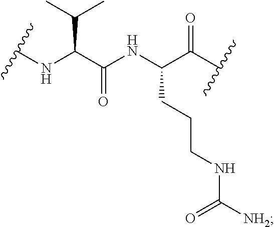 [SMILES: *N[C@H](C(=O)N[C@@H](CCCNC(N)=O)C(*)=O)C(C)C]

the —SP¹— spacer is

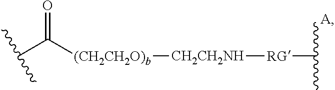

wherein RG′ is a reactive group residue following reaction of a reactive group RG with a binding agent;

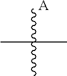

is a bond, direct or indirect, to the binding agent; and b is an integer from one to four. In certain embodiments, p is as described above. In certain embodiments, b is one. In certain embodiments, b is two. In certain embodiments, b is three. In certain embodiments, b is four. In certain embodiments, Q is —O—. In certain embodiments, the conjugate has a structure of Formula A′, B′, C′, D′, or E′, wherein Q is —CH₂—; X is —NR⁵; R⁵ is —CH₃ or —(CH₂)₂—OH; R³ is —C₅ alkyl; R⁶ is —OH; R⁷ is —CH₃; and r is four. In one embodiment, the conjugate has a structure of Formula C′, or a pharmaceutically acceptable salt thereof. In one embodiment, the conjugate has a structure of Formula C′, or a pharmaceutically acceptable salt thereof, wherein R³ is —NH—(CH₂)₂OH, —NH—CH₂—C(O)—NH₂, —NH—C(O)—CH₂NH₂, or —NH—CH₂—C(O)—NH—(CH₂CH₂O)₂—(CH₂)₂NH₂; and R⁴ is hydrogen. In one embodiment, the conjugate has a structure of Formula C′, or a pharmaceutically acceptable salt thereof, wherein R³ is —NH—(CH₂)₂O—, —NH—CH₂—C(O)—NH—, —NH—C(O)—CH₂NH—, or —NH—CH₂—C(O)—NH—(CH₂CH₂O)₂—(CH₂)₂NH—; and R⁴ is hydrogen. In one embodiment, the conjugate has a structure of Formula C′, or a pharmaceutically acceptable salt thereof, wherein R³ is —NH—(CH₂)₂OH. In one embodiment, the conjugate has a structure of Formula C′, or a pharmaceutically acceptable salt thereof, wherein R³ is —NH—(CH₂)₂O—. In one embodiment, the conjugate has a structure of Formula C′, or a pharmaceutically acceptable salt thereof, wherein R³ is —NH—CH₂—C(O)—NH₂. In one embodiment, the conjugate has a structure of Formula C′, or a pharmaceutically acceptable salt thereof, wherein R³ is —NH—CH₂—C(O)—NH—. In one embodiment, the conjugate has a structure of Formula C′, or a pharmaceutically acceptable salt thereof, wherein R³ is —NH—C(O)—CH₂NH₂. In one embodiment, the conjugate has a structure of Formula C′, or a pharmaceutically acceptable salt thereof, wherein R³ is —NH—C(O)—CH₂NH—. In one embodiment, the conjugate has a structure of Formula C′, or a pharmaceutically acceptable salt thereof, wherein R³ is —NH—CH₂—C(O)—NH—(CH₂CH₂O)₂—(CH₂)₂NH₂. In one embodiment, the conjugate has a structure of Formula C′, or a pharmaceutically acceptable salt thereof, wherein R³ is —NH—CH₂—C(O)—NH—(CH₂CH₂O)₂—(CH₂)₂NH—. In certain embodiments, the conjugate has a structure of Formula A′, B′, C′, D′, or E′, wherein Q is —CH₂—; X is —NR⁵, R⁵ is —CH₃ or —(CH₂)₂—OH; R⁴ is —C₅ alkyl; R⁶ is —OH; R⁷ when present is —CH₃; and r is four. In one embodiment, the conjugate has a structure of Formula C′, or a pharmaceutically acceptable salt thereof. In certain embodiments, the conjugate has a structure of Formula A′, B′, C′, D′, or E′, wherein Q is —CH₂—; X is —NR⁵, R⁴ is —C₅ alkyl; R⁶ is —OH; R⁷ when present is —CH₃; and r is four. In one embodiment, the conjugate has a structure of Formula A′, or a pharmaceutically acceptable salt thereof. In one embodiment, the conjugate has a structure of Formula A′, or a pharmaceutically acceptable salt thereof, wherein R⁵ is —C(O)—CH₂—NH₂. In one embodiment, the conjugate has a structure of Formula A′, or a pharmaceutically acceptable salt thereof, wherein R⁵ is —C(O)—CH₂—NH—. In certain embodiments, the conjugate has a structure of Formula A′, B′, C′, D′, or E′, wherein Q is —CH₂—; X is —NR⁵, R⁵ is —CH₃ or —(CH₂)₂—OH; R⁴ is —C₅ alkyl; R⁶ is —OH; R⁷ when present is —CH₃; and r is four. In one embodiment, the conjugate has a structure of Formula C′, or a pharmaceutically acceptable salt thereof. In certain embodiments, the conjugate has a structure of Formula A′, B′, C′, D′, or E′, wherein Q is —CH₂—; X is —NR⁵, R⁵ is —CH₃; R⁴ is −C₅ alkyl; R⁶ is —OH; R⁷ when present is —CH₃; and r is four. In one embodiment, the conjugate has a structure of Formula E′, or a pharmaceutically acceptable salt thereof. In one embodiment, the conjugate has a structure of Formula E′, or a pharmaceutically acceptable salt thereof, wherein R² is —O—C(O)—NH—(CH₂CH₂O)₂—(CH₂)₂OH. In one embodiment, the conjugate has a structure of Formula E′, or a pharmaceutically acceptable salt thereof, wherein R² is —O—C(O)—NH—(CH₂CH₂O)₂—(CH₂)₂O—. In one embodiment, the conjugate has a structure of Formula C′, or a pharmaceutically acceptable salt thereof, wherein R³ is —NH—(CH₂)₂OH; and R⁴ is hydrogen. In one embodiment, the conjugate has a structure of Formula C′, or a pharmaceutically acceptable salt thereof, wherein R³ is —NH—(CH₂)₂O—; and R⁴ is hydrogen. In one embodiment, the conjugate has a structure of Formula C′, or a pharmaceutically acceptable salt thereof, wherein R³ is —NH—CH₂—C(O)—NH₂; and R⁴ is hydrogen. In one embodiment, the conjugate has a structure of Formula C′, or a pharmaceutically acceptable salt thereof, wherein R³ is —NH—CH₂—C(O)—NH—; and R⁴ is hydrogen. In one embodiment, the conjugate has a structure of Formula C′, or a pharmaceutically acceptable salt thereof, wherein R³ is —NH—C(O)—CH₂NH₂; and R⁴ is hydrogen. In one embodiment, the conjugate has a structure of Formula C′, or a pharmaceutically acceptable salt thereof, wherein R³ is —NH—C(O)—CH₂NH—; and R⁴ is hydrogen. In one embodiment, the conjugate has a structure of Formula C′, or a pharmaceutically acceptable salt thereof, wherein R³ is —NH—CH₂—C(O)—NH—(CH₂CH₂O)₂—(CH₂)₂NH₂; and R⁴ is hydrogen. In one embodiment, the conjugate has a structure of Formula C′, or a pharmaceutically acceptable salt thereof, wherein R³ is —NH—CH₂—C(O)—NH—(CH₂CH₂O)₂—(CH₂)₂NH—; and R⁴ is hydrogen.

Provided herein are conjugates of Formula A. In certain embodiments, compounds conjugated to -L-BA in Formula A include one or more compounds of Formulae I, Ia, Iaa, II, III, IV, V, and/or VI as described above, wherein BA is a binding agent; L is a linker; and k is one, two, three, four, five, six, seven, eight, nine, or ten. In certain embodiments, k is a range from 1-2, 1-3, 2-3, 2-4, 3-4, or 1-4. In any embodiment in this paragraph, BA is antibody or antigen binding fragment thereof, wherein the antibody is conjugated to a compound of Formula I, as described above. In any embodiment in this paragraph, BA is antibody or antigen binding fragment thereof, wherein the antibody is conjugated to a compound of Formula Ia, as described above. In any embodiment in this paragraph, BA is antibody or antigen binding fragment thereof, wherein the antibody is conjugated to a compound of Formula Iaa, as described above. In any embodiment in this paragraph, BA is antibody or antigen binding fragment thereof, wherein the antibody is conjugated to a compound of Formula II, as described above. In any embodiment in this paragraph, BA is antibody or antigen binding fragment thereof, wherein the antibody is conjugated to a compound of Formula III, as described above. In any embodiment in this paragraph, BA is antibody or antigen binding fragment thereof, wherein the antibody is conjugated to a compound of Formula IV, as described above. In any embodiment in this paragraph, BA is antibody or antigen binding fragment thereof, wherein the antibody is conjugated to a compound of Formula V, as described above. In any embodiment in this paragraph, BA is antibody or antigen binding fragment thereof, wherein the antibody is conjugated to a compound of Formula VI, as described above. In any of the embodiments in this paragraph, any one or more compounds of Formulae I, Ia, Iaa, II, III, IV, V, and/or VI conjugated to -L-BA in Formula A are conjugated via the heterocycle comprising nitrogen, as described elsewhere herein. In certain embodiments, when Q is —O—, then IV is C₁-C₈ alkyl or C₂-C₈ alkynyl. In certain embodiments in this paragraph, Q¹ is —CH₂—. In certain embodiments in this paragraph, Q¹ is —O—. In certain embodiments, when Q is —CH₂—, then IV is C₁-C₈ alkyl or C₂-C₈ alkynyl.

Provided herein are conjugates of Formula B. In certain embodiments, compounds conjugated to -L-BA in Formula B include one or more compounds of Formulae I, Ia, Iaa, II, III, IV, V, and/or VI, as described above, wherein BA is a binding agent; L is a linker; and k is one, two, three, four, five, six, seven, eight, nine, or ten. In certain embodiments, k is a range from 1-2, 1-3, 2-3, 2-4, 3-4, or 1-4. In any embodiment in this paragraph, BA is antibody or antigen binding fragment thereof, wherein the antibody is conjugated to a compound of Formula I, as described above. In any embodiment in this paragraph, BA is antibody or antigen binding fragment thereof, wherein the antibody is conjugated to a compound of Formula Ia, as described above. In any embodiment in this paragraph, BA is antibody or antigen binding fragment thereof, wherein the antibody is conjugated to a compound of Formula Iaa, as described above. In any embodiment in this paragraph, BA is antibody or antigen binding fragment thereof, wherein the antibody is conjugated to a compound of Formula II, as described above. In any embodiment in this paragraph, BA is antibody or antigen binding fragment thereof, wherein the antibody is conjugated to a compound of Formula III, as described above. In any embodiment in this paragraph, BA is antibody or antigen binding fragment thereof, wherein the antibody is conjugated to a compound of Formula IV, as described above. In any embodiment in this paragraph, BA is antibody or antigen binding fragment thereof, wherein the antibody is conjugated to a compound of Formula V, as described above. In any embodiment in this paragraph, BA is antibody or antigen binding fragment thereof, wherein the antibody is conjugated to a compound of Formula VI, as described above. In any of the embodiments in this paragraph, any one or more compounds of Formulae I, Ia, Iaa, II, III, IV, V, and/or VI conjugated to -L-BA in Formula B are conjugated via divalent R⁶. In certain embodiments, when Q is —O—, then R¹ is C₁-C₈ alkyl or C₂-C₈ alkynyl. In certain embodiments in this paragraph, Q¹ is —CH₂—. In certain embodiments in this paragraph, Q¹ is —O—. In certain embodiments, when Q is —CH₂—, then R³ is C₁-C₈ alkyl or C₂-C₈ alkynyl.

Provided herein are conjugates of Formula C. In certain embodiments, compounds conjugated to -L-BA in Formula C include one or more compounds of Formulae I, Ia, Iaa, II, III, IV, V, and/or VI as described above, wherein BA is a binding agent; L is a linker; and k is one, two, three, four, five, six, seven, eight, nine, or ten. In certain embodiments, k is a range from 1-2, 1-3, 2-3, 2-4, 3-4, or 1-4. In any embodiment in this paragraph, BA is antibody or antigen binding fragment thereof, wherein the antibody is conjugated to a compound of Formula I, as described above. In any embodiment in this paragraph, BA is antibody or antigen binding fragment thereof, wherein the antibody is conjugated to a compound of Formula Ia, as described above. In any embodiment in this paragraph, BA is antibody or antigen binding fragment thereof, wherein the antibody is conjugated to a compound of Formula Iaa, as described above. In any embodiment in this paragraph, BA is antibody or antigen binding fragment thereof, wherein the antibody is conjugated to a compound of Formula II, as described above. In any embodiment in this paragraph, BA is antibody or antigen binding fragment thereof, wherein the antibody is conjugated to a compound of Formula III, as described above. In any embodiment in this paragraph, BA is antibody or antigen binding fragment thereof, wherein the antibody is conjugated to a compound of Formula IV, as described above. In any embodiment in this paragraph, BA is antibody or antigen binding fragment thereof, wherein the antibody is conjugated to a compound of Formula V, as described above. In any embodiment in this paragraph, BA is antibody or antigen binding fragment thereof, wherein the antibody is conjugated to a compound of Formula VI, as described above. In any of the embodiments in this paragraph, any one or more compounds of Formulae I, Ia, Iaa, II, III, IV, V, and/or VI conjugated to -L-BA in Formula C are conjugated via divalent R³. In certain embodiments, when Q is —O—, then R¹ is C₁-C₈ alkyl or C₂-C₈ alkynyl. In certain embodiments in this paragraph, Q¹ is —CH₂—. In certain embodiments in this paragraph, Q¹ is —O—. In certain embodiments, when Q is —CH₂—, then R³ is C₁-C₈ alkyl or C₂-C₈ alkynyl.

Provided herein are conjugates of Formula D. In certain embodiments, compounds conjugated to -L-BA in Formula D include one or more compounds of Formulae I, Ia, Iaa, II, III, IV, V, and/or VI as described above, wherein BA is a binding agent; L is a linker; and k is one, two, three, four, five, six, seven, eight, nine, or ten. In certain embodiments, k is a range from 1-2, 1-3, 2-3, 2-4, 3-4, or 1-4. In any embodiment in this paragraph, BA is antibody or antigen binding fragment thereof, wherein the antibody is conjugated to a compound of Formula I, as described above. In any embodiment in this paragraph, BA is antibody or antigen binding fragment thereof, wherein the antibody is conjugated to a compound of Formula Ia, as described above. In any embodiment in this paragraph, BA is antibody or antigen binding fragment thereof, wherein the antibody is conjugated to a compound of Formula Iaa, as described above. In any embodiment in this paragraph, BA is antibody or antigen binding fragment thereof, wherein the antibody is conjugated to a compound of Formula II, as described above. In any embodiment in this paragraph, BA is antibody or antigen binding fragment thereof, wherein the antibody is conjugated to a compound of Formula III, as described above. In any embodiment in this paragraph, BA is antibody or antigen binding fragment thereof, wherein the antibody is conjugated to a compound of Formula IV, as described above. In any embodiment in this paragraph, BA is antibody or antigen binding fragment thereof, wherein the antibody is conjugated to a compound of Formula V, as described above. In any embodiment in this paragraph, BA is antibody or antigen binding fragment thereof, wherein the antibody is conjugated to a compound of Formula VI, as described above. In any of the embodiments in this paragraph, any one or more compounds of Formulae I, Ia, Iaa, II, III, IV, V, and/or VI conjugated to -L-BA in Formula D are conjugated via divalent R′. In certain embodiments, when Q is —O—, then R¹ is C₁-C₈ alkyl or C₂-C₈ alkynyl. In certain embodiments in this paragraph, Q¹ is —CH₂—. In certain embodiments in this paragraph, Q¹ is —O—. In certain embodiments, when Q is —CH₂—, then R¹ is C₁-C₈ alkyl or C₂-C₈ alkynyl.

Provided herein are conjugates of Formula E. In certain embodiments, compounds conjugated to -L-BA in Formula E include one or more compounds of Formulae I, IA, Iaa, II, III, IV, V, and/or VI as described above, wherein BA is a binding agent; L is a linker; and k is one, two, three, four, five, six, seven, eight, nine, or ten. In certain embodiments, k is a range from 1-2, 1-3, 2-3, 2-4, 3-4, or 1-4. In any embodiment in this paragraph, BA is antibody or antigen binding fragment thereof, wherein the antibody is conjugated to a compound of Formula I, as described above. In any embodiment in this paragraph, BA is antibody or antigen binding fragment thereof, wherein the antibody is conjugated to a compound of Formula Ia, as described above. In any embodiment in this paragraph, BA is antibody or antigen binding fragment thereof, wherein the antibody is conjugated to a compound of Formula Iaa, as described above. In any embodiment in this paragraph, BA is antibody or antigen binding fragment thereof, wherein the antibody is conjugated to a compound of Formula II, as described above. In any embodiment in this paragraph, BA is antibody or antigen binding fragment thereof, wherein the antibody is conjugated to a compound of Formula III, as described above. In any embodiment in this paragraph, BA is antibody or antigen binding fragment thereof, wherein the antibody is conjugated to a compound of Formula IV, as described above. In any embodiment in this paragraph, BA is antibody or antigen binding fragment thereof, wherein the antibody is conjugated to a compound of Formula V, as described above. In any embodiment in this paragraph, BA is antibody or antigen binding fragment thereof, wherein the antibody is conjugated to a compound of Formula VI, as described above. In any of the embodiments in this paragraph, any one or more compounds of Formulae I, II, III, IV, V, and/or VI conjugated to -L-BA in Formula E are conjugated via divalent R². In certain embodiments, when Q is —O—, then R¹ is C₁-C₈ alkyl or C₂-C₈ alkynyl. In certain embodiments in this paragraph, Q¹ is —CH₂—. In certain embodiments in this paragraph, Q¹ is —O—. In certain embodiments, when Q is —CH₂—, then R¹ is C₁-C₈ alkyl or C₂-C₈ alkynyl.

In certain embodiments, the compound of Formula A′, B′, C′, D′, or E′ is selected from the group consisting of

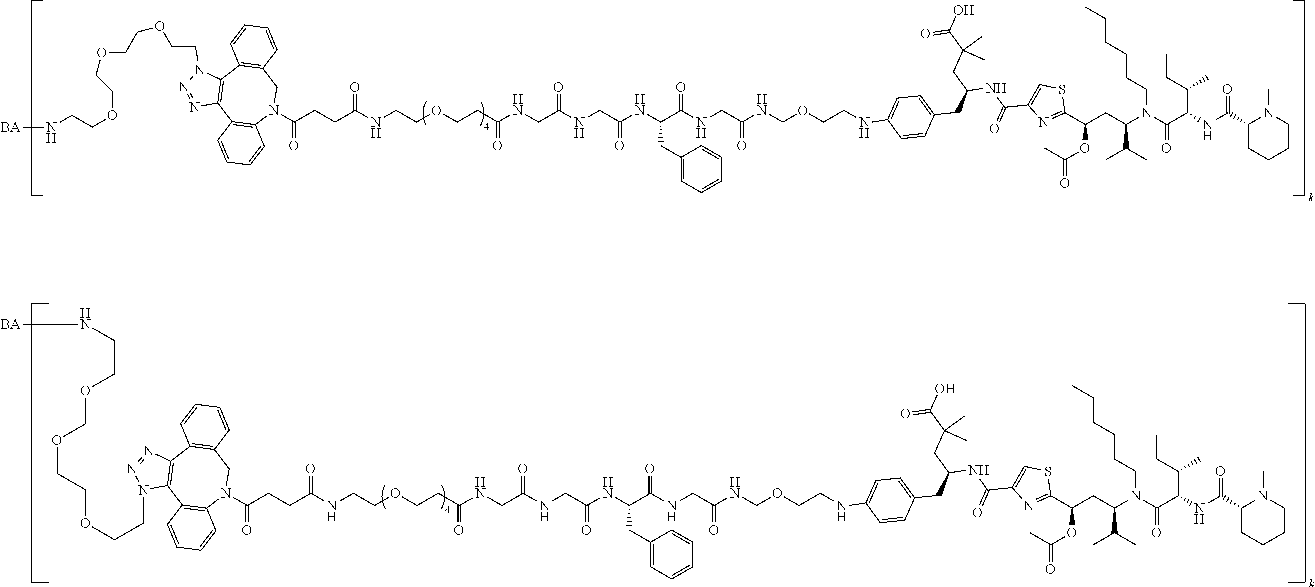

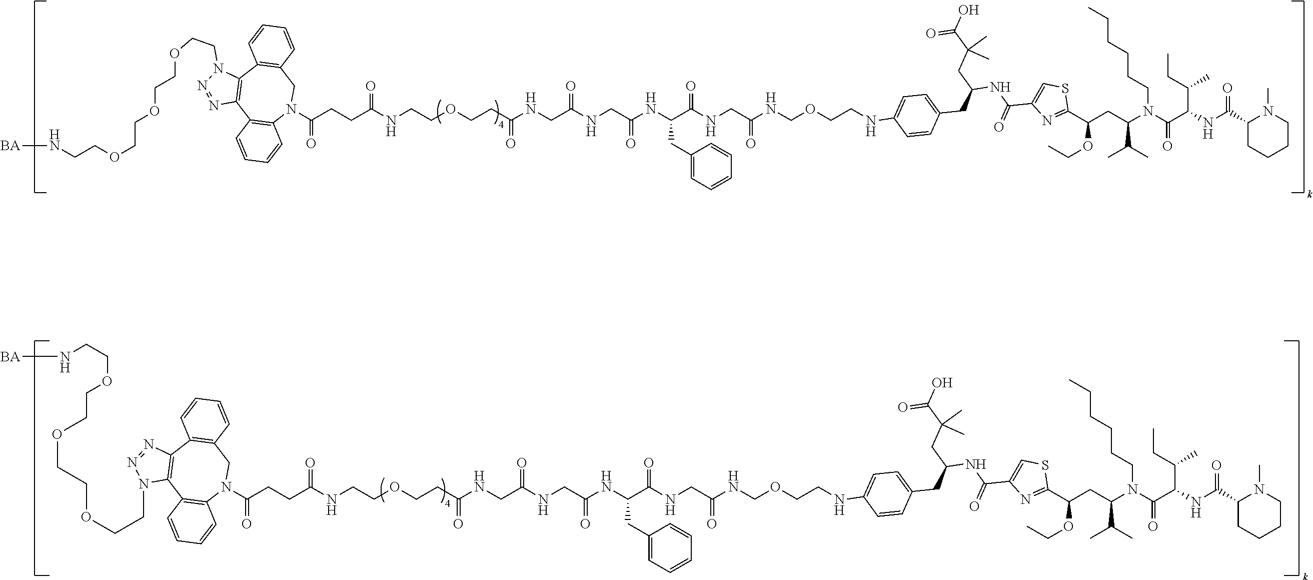

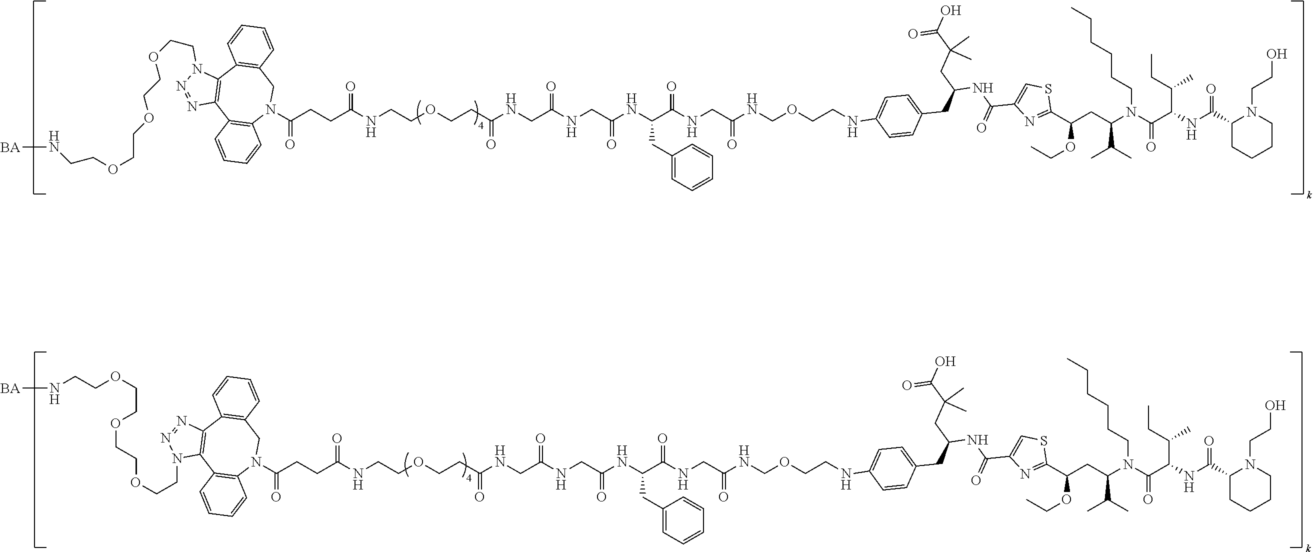

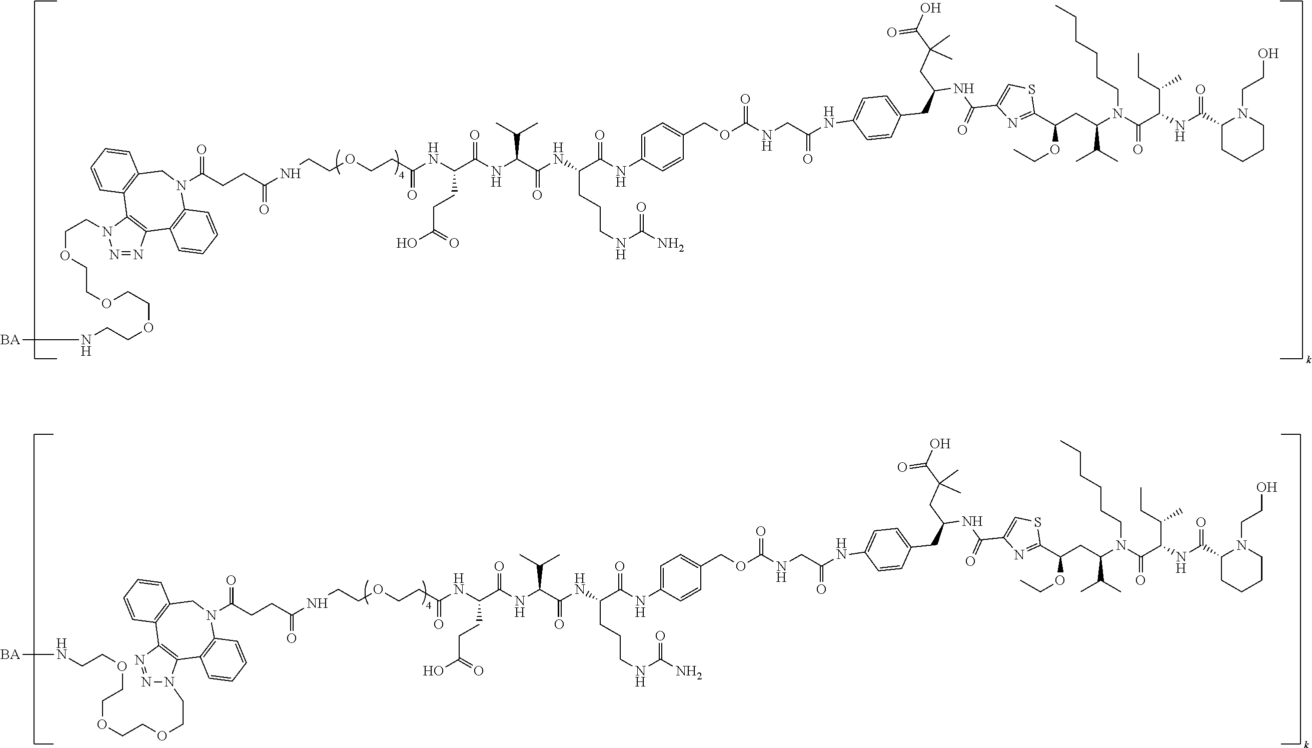

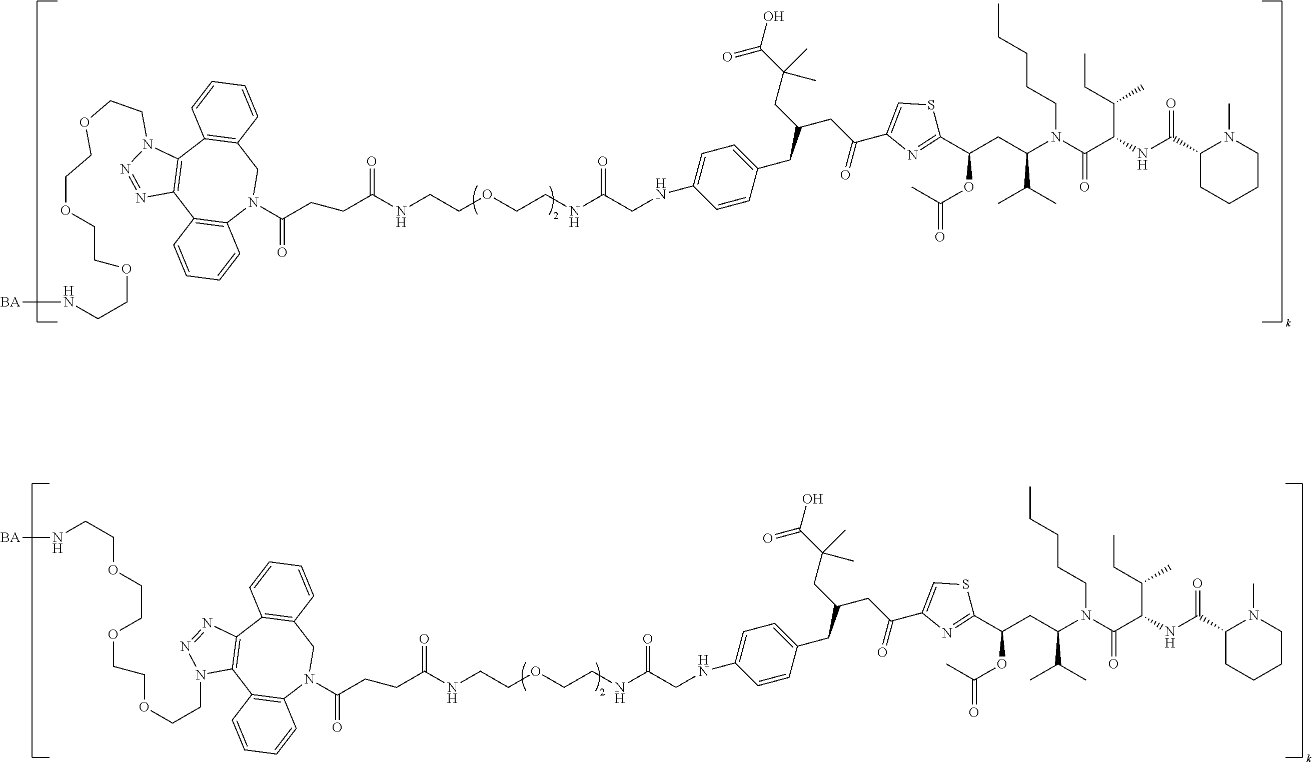

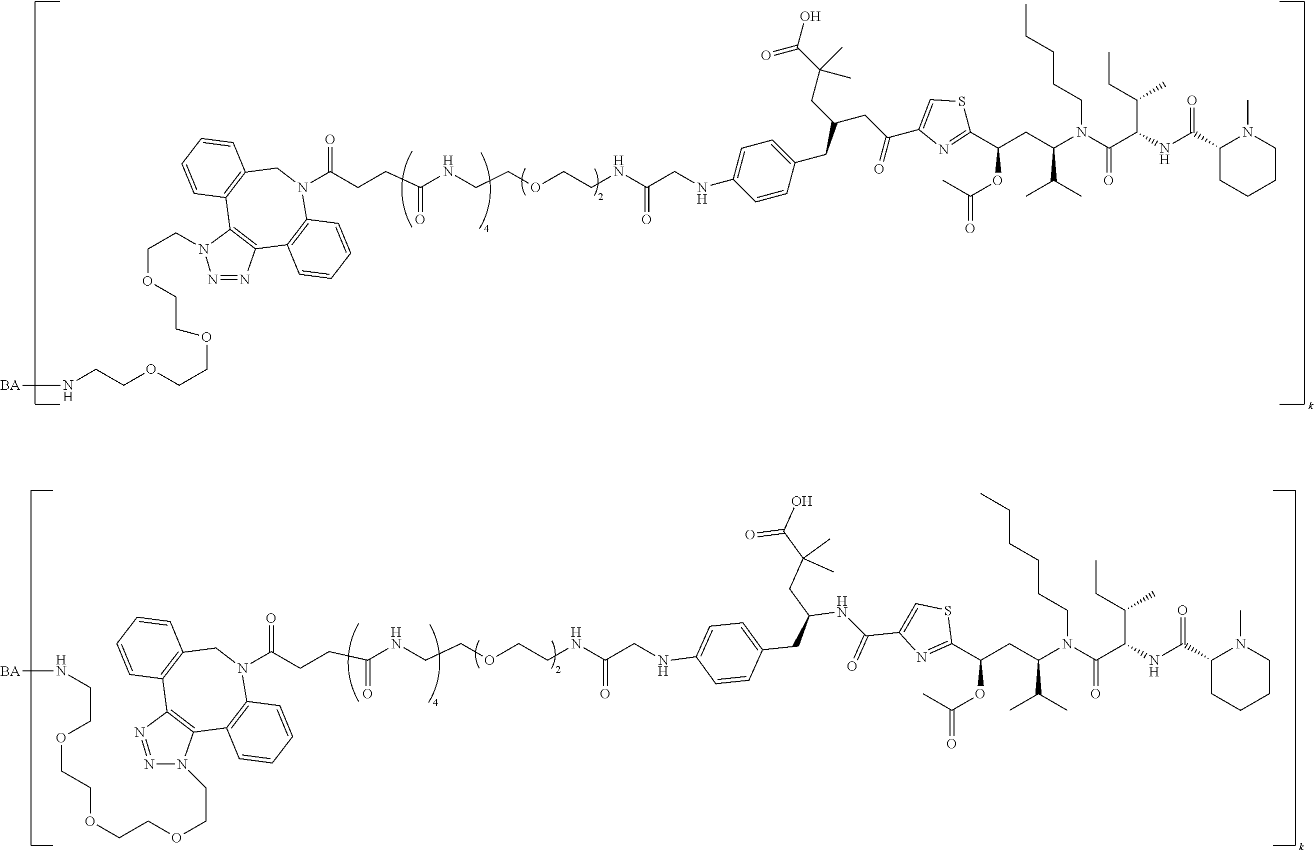

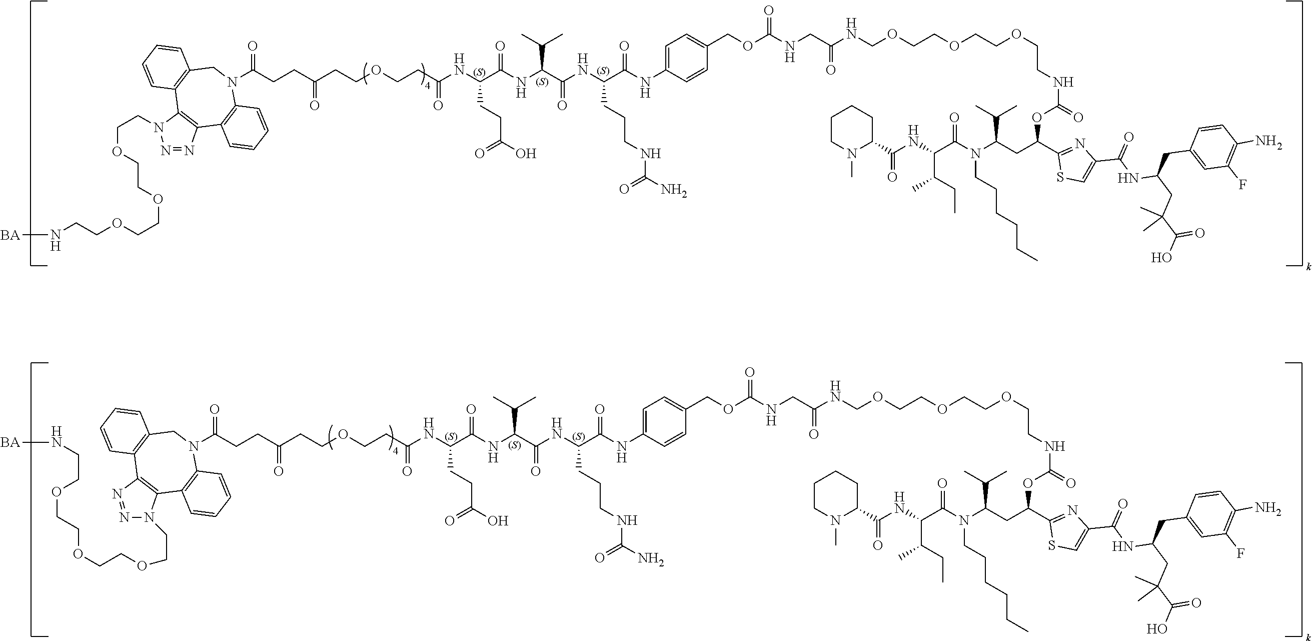

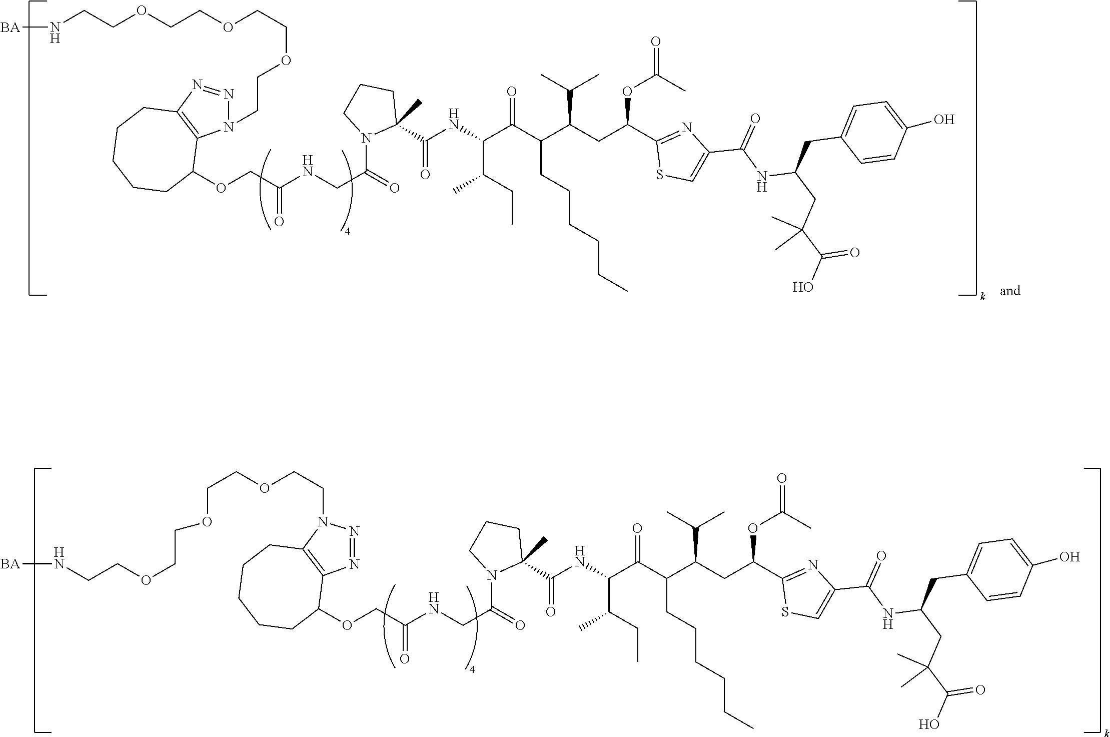

or a pharmaceutically acceptable salt thereof, wherein BA is a binding agent; and k is one, two, three, or four.

In certain embodiments, the compound is selected from the group consisting of

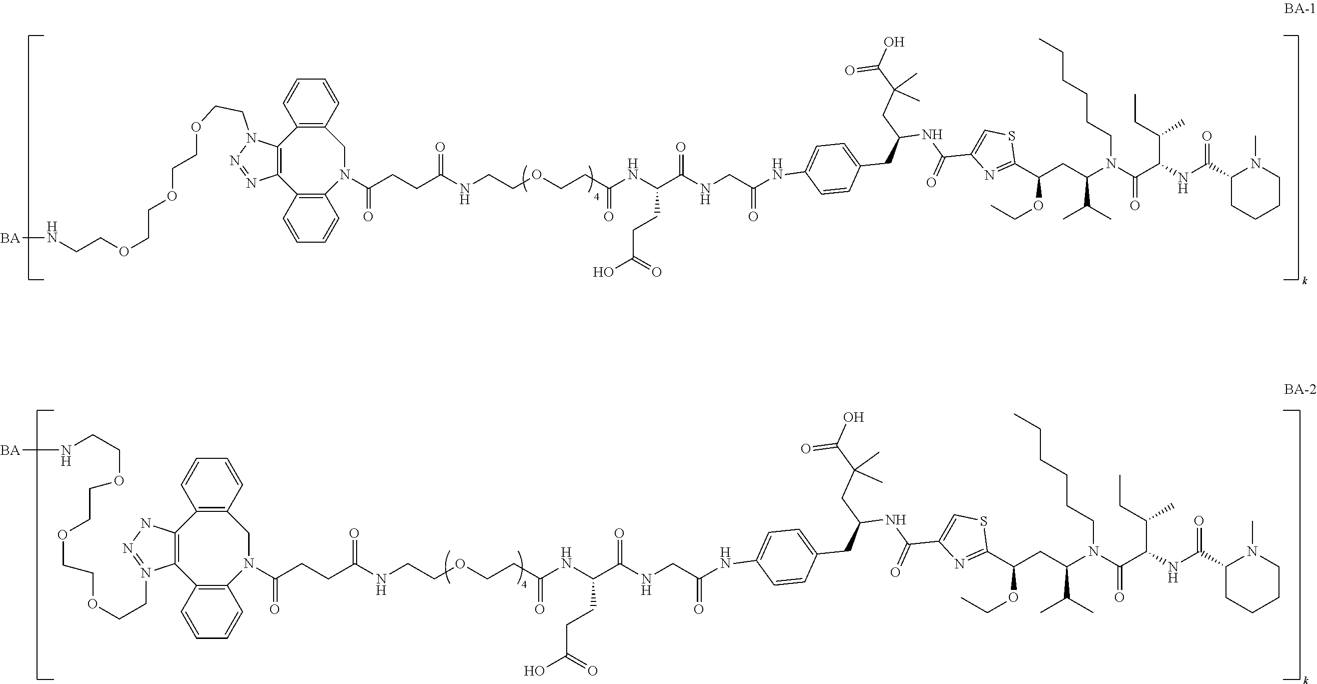

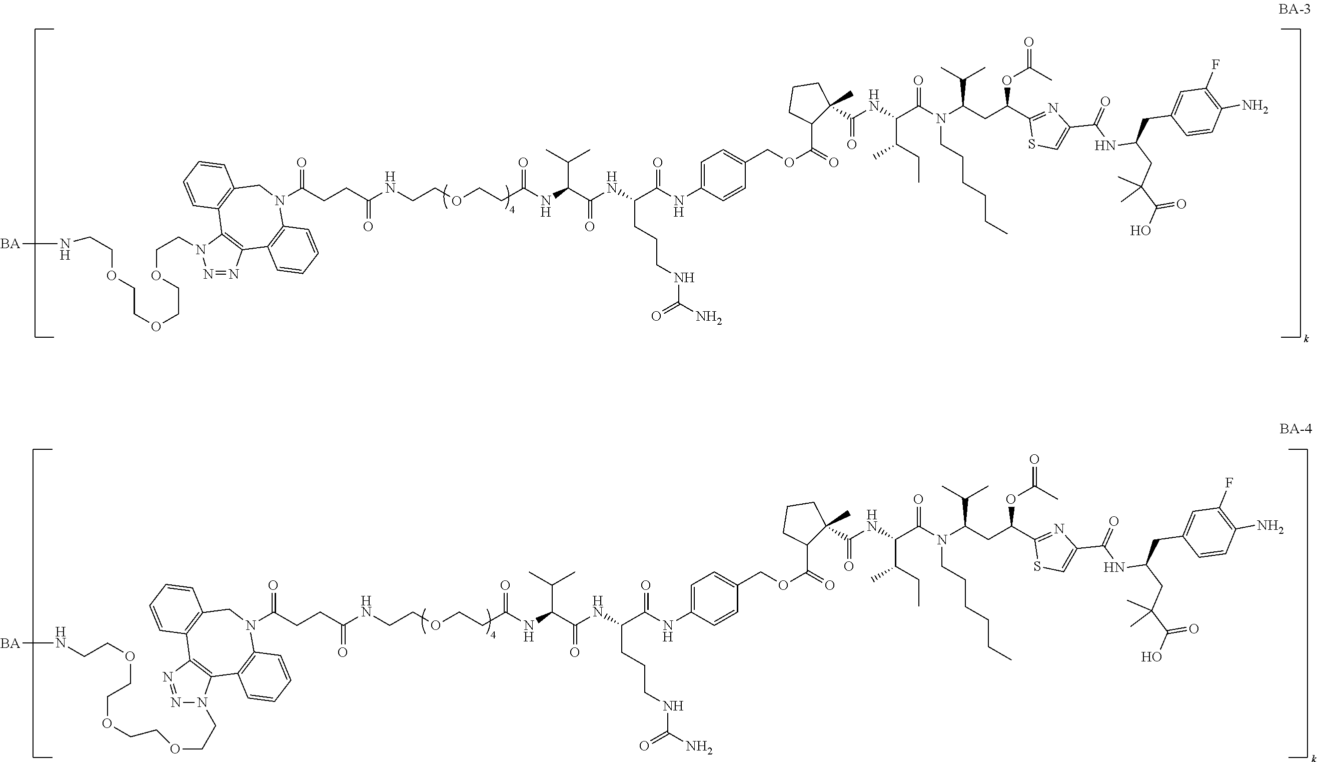

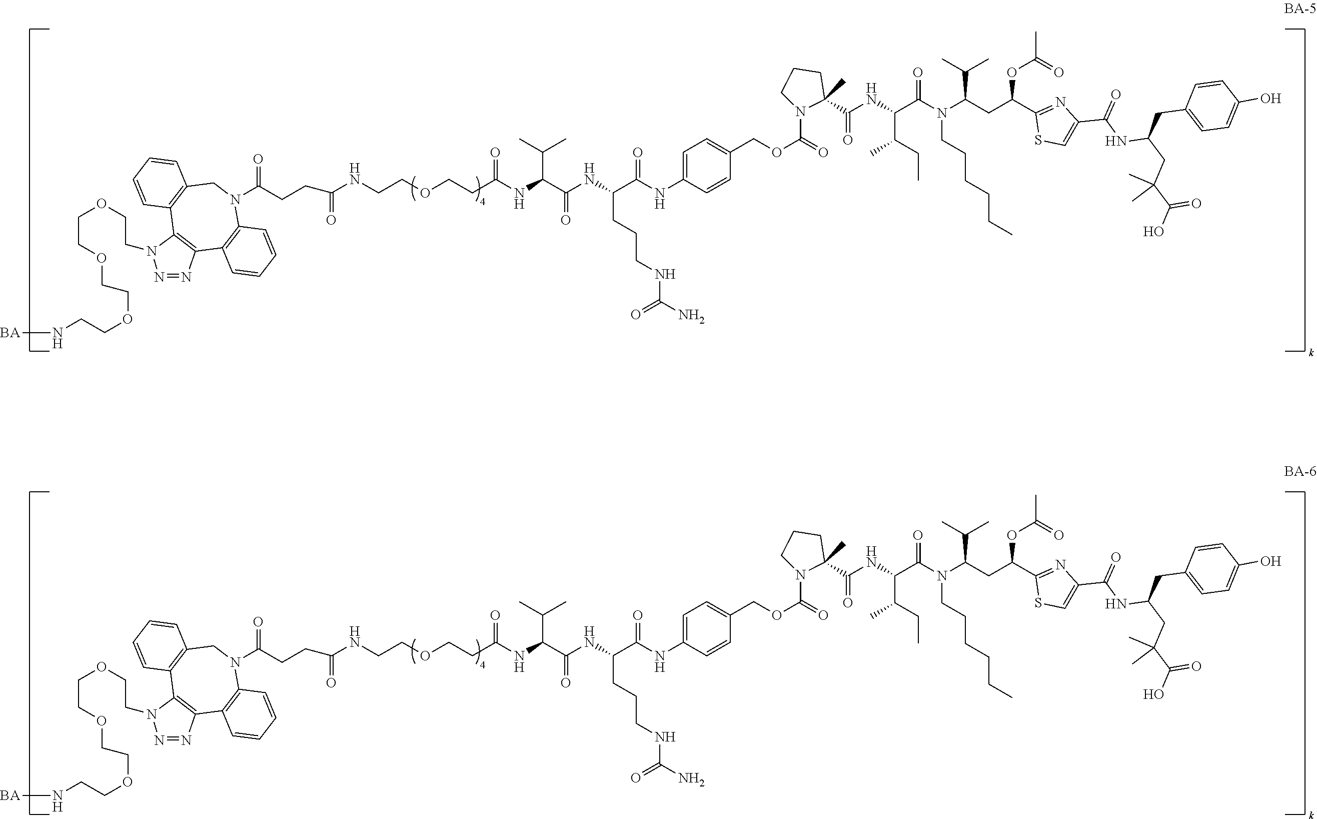

or a pharmaceutically acceptable salt thereof, wherein BA is a binding agent; and k is one, two, three, or four.

In certain embodiments, an antibody or antigen-binding fragment thereof can be conjugated directly, or via a linker, to any one or more of Formulae I, Ia, Iaa, II, III, IV, V, and/or VI as described herein. In one embodiment, an antibody-drug conjugate includes an antibody or antigen binding fragment thereof conjugated to any one or more of Formulae I, Ia, Iaa, II, III, IV, V, and/or VI as described herein, selected from the group consisting of

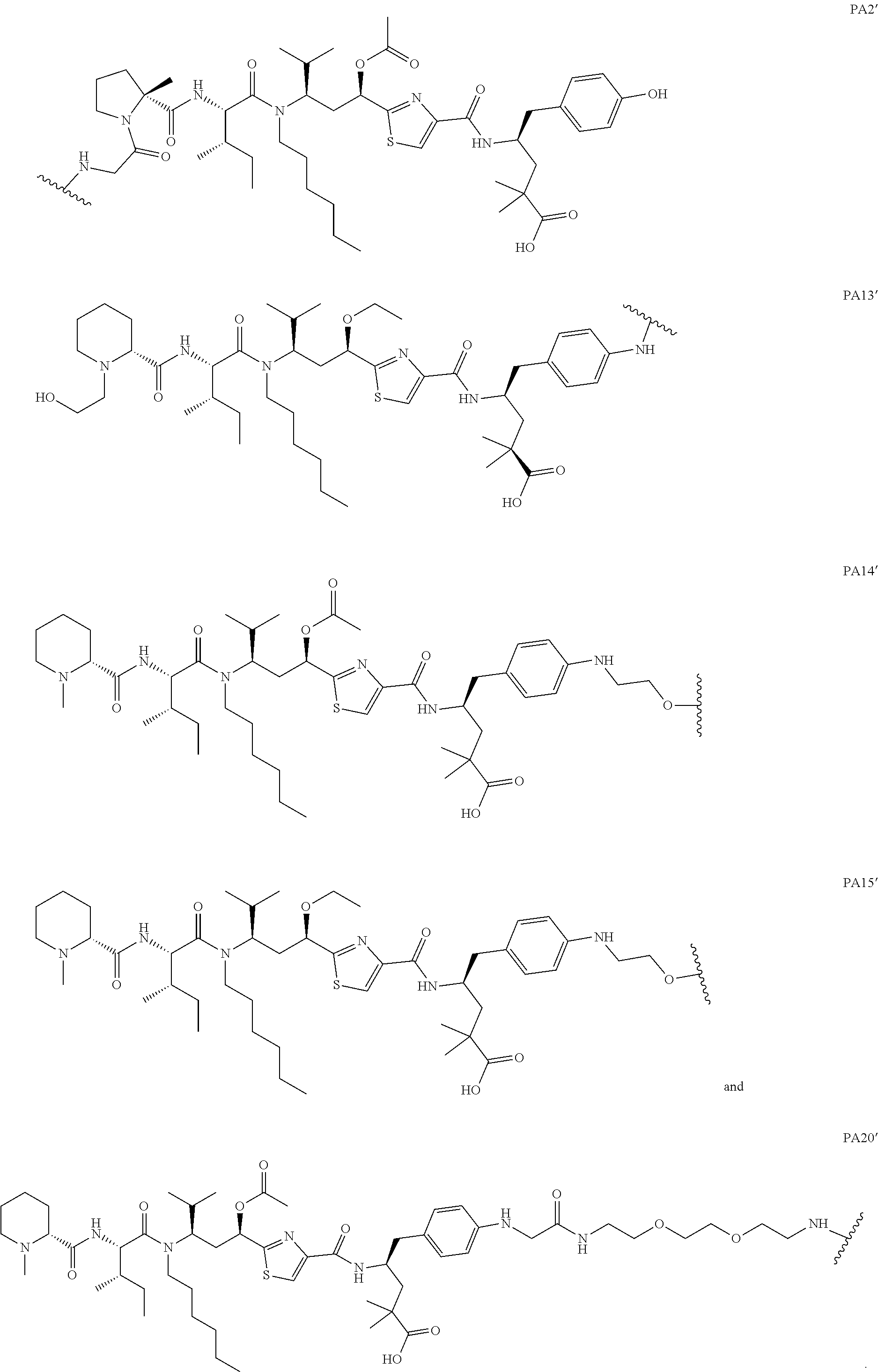

In one embodiment, an antibody-drug conjugate includes an antibody or antigen binding fragment thereof conjugated to any one or more of Formulae I, Ia, Iaa, II, III, IV, V, and/or VI as described herein, selected from the group consisting of

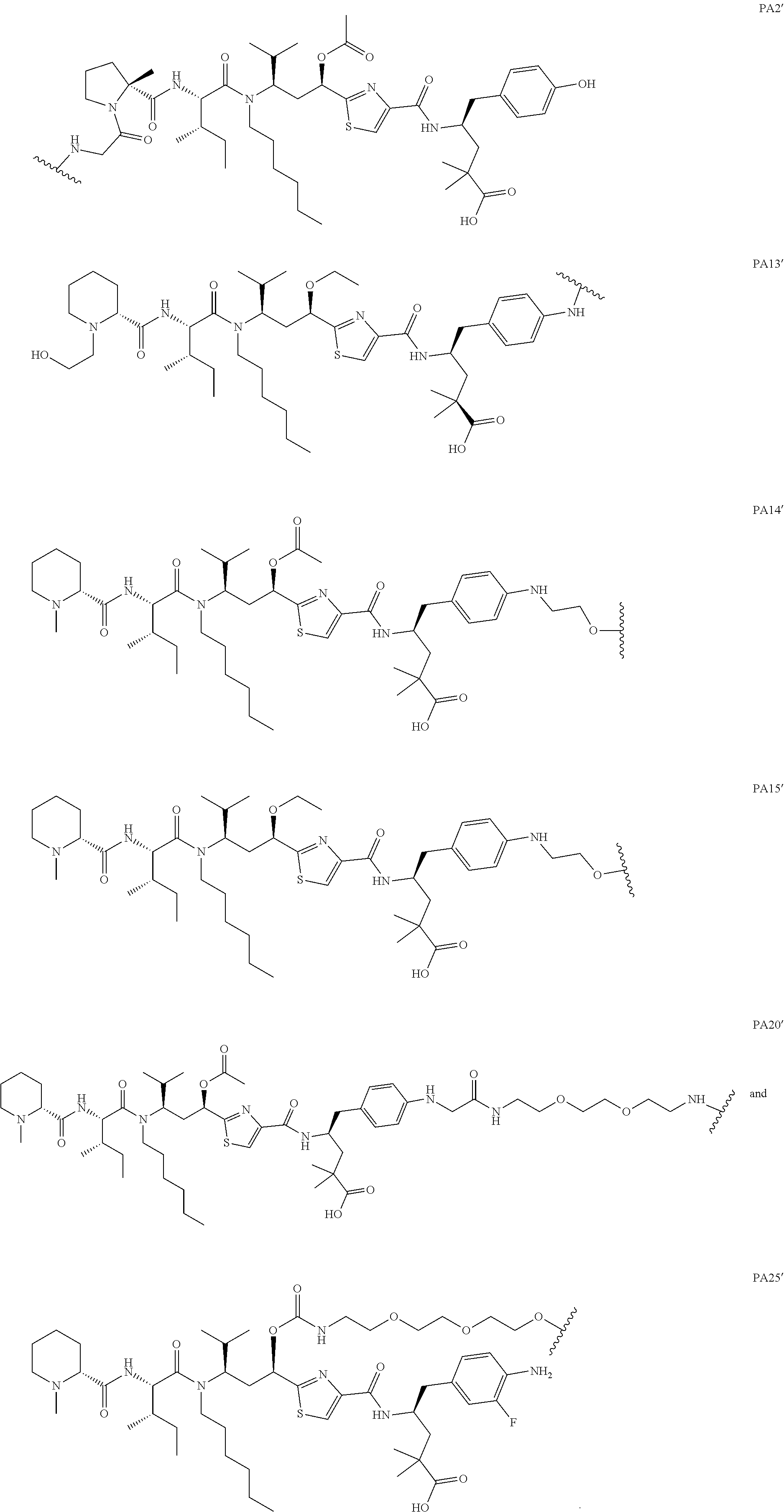

In any of the compound or conjugate embodiments provided, BA is an antibody or antigen binding fragment thereof. In any of the compound or conjugate embodiments provided, BA is a transglutaminase-modified antibody or antigen-binding fragment thereof comprising at least one glutamine residue used for conjugation. In any of the compound or conjugate embodiments provided, BA is a transglutaminase-modified antibody or antigen-binding fragment thereof comprising at least two glutamine residues used for conjugation. In any of the compound or conjugate embodiments provided, BA is a transglutaminase-modified antibody or antigen-binding fragment thereof comprising at least four glutamine residues used for conjugation. In any of the compound or conjugate embodiments provided, BA is a transglutaminase-modified antibody or antigen-binding fragment thereof wherein conjugation is at two Q295 residues; and k is two. In any of the compound or conjugate embodiments provided, BA is a transglutaminase-modified antibody or antigen-binding fragment thereof wherein conjugation is at two Q295 residues and two N297Q residues; and k is four. In any of the compound or conjugate embodiments provided, BA is an antibody or antigen binding fragment thereof that binds PRLR. In any of the compound or conjugate embodiments provided, BA is an antibody or antigen binding fragment thereof that binds STEAP2. In any of the compound or conjugate embodiments provided, BA is an antibody or antigen-binding fragment thereof and conjugation is through at least one Q295 residue. In any of the compound or conjugate embodiments provided, BA is an antibody or antigen-binding fragment thereof, and conjugation is through two Q295 residues. In any of the compound or conjugate embodiments provided, BA is a N297Q antibody or antigen-binding fragment thereof. In any of the compound or conjugate embodiments provided, BA is a N297Q antibody or antigen-binding fragment thereof, and conjugation is through at least one Q295 and at least one Q297 residue. In any of the compound or conjugate embodiments provided, BA is a N297Q antibody or antigen-binding fragment thereof, and conjugation is through two Q295 residues and two Q297 residues. In particular embodiments, numbering is according to the EU numbering system. In one embodiment, BA or the antibody or antigen-binding fragment thereof is selected from the group consisting of anti-MUC16, anti-PSMA, anti-EGFRvIII, anti-HER2, and anti-MET. In one embodiment, BA or the antibody or antigen-binding fragment thereof is anti-PRLR or anti-STEAP2. In one embodiment, BA or the antibody or antigen-binding fragment thereof binds to an antigen selected from the group consisting of lipoproteins; alpha1-antitrypsin; a cytotoxic T-lymphocyte associated antigen (CTLA), such as CTLA-4 or CTLA4; vascular endothelial growth factor (VEGF); receptors for hormones or growth factors; protein A or D; fibroblast growth factor receptor 2 (FGFR2), EpCAM or Epcam, GD3, FLT3, PSCA, MUC1 or Muc1, MUC16 or Muc16, STEAP, STEAP2 or Steap-2, CEA, TENB2, EphA receptors, EphB receptors, folate receptor, FOLRI, mesothelin, cripto, alphavbeta6, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152; erythropoietin; osteoinductive factors; immunotoxins; a bone morphogenetic protein (BMP); T-cell receptors; surface membrane proteins; integrins, such as CD11a, CD11b, CD11c, CD18, an ICAM, VLA-4, and VCAM; a tumor associated antigen such as AFP, ALK, B7H4, BAGE proteins, β-catenin, brc-abl, BRCA1, BORIS, CA9 (carbonic anhydrase IX), caspase-8, CD123, CDK4, CLEC12A, c-kit, cMET, c-MET, MET, cyclin-B1, CYP1B1, EGFRvIII, endoglin, EphA2, ErbB2/Her2, ErbB3/Her3, ErbB4/Her4, ETV6-AML, Fra-1, FOLR1, GAGE proteins such as GAGE-1 and GAGE-2, GD2, GloboH, glypican-3, GM3, gp100, Her2 or HER2, HLA/B-raf, HLA/EBNA1, HLA/k-ras, HLA/MAGE-A3, hTERT, IGF1R, LGR5, LMP2, MAGE proteins such as MAGE-1, -2, -3, -4, -6, and -12, MART-1, ML-IAP, CA-125, MUM1, NA17, NGEP, NY-BR1, NY-BR62, NY-BR85, NY-ESO1, OX40, p15, p53, PAP, PAX3, PAX5, PCTA-1, PDGFR-α, PDGFR-β, PDGF-A, PDGF-B, PDGF-C, PDGF-D, PLAC1, PRLR, PRAME, PSGR, PSMA (FOLH1), RAGE proteins, Ras, RGS5, Rho, SART-1, SART-3, Steap-1, STn, survivin, TAG-72, TGF-β, TMPRSS2, Tn, TNFRSF17, TRP-1, TRP-2, tyrosinase, uroplakin-3, fragments of any of the above-listed polypeptides; cell-surface expressed antigens; molecules such as class A scavenger receptors including scavenger receptor A (SR-A), and other membrane proteins such as B7 family-related member including V-set and Ig domain-containing 4 (VSIG4), Colony stimulating factor 1 receptor (CSF1R), asialoglycoprotein receptor (ASGPR), and Amyloid beta precursor-like protein 2 (APLP-2); BCMA; SLAMF7; GPNMB; and UPK3A.

In any of the embodiments above, BA is an anti-STEAP2 antibody. In certain embodiments, BA is the anti-STEAP2 antibody H1H7814N described in the Examples below. In certain embodiments, BA is the anti-STEAP2 antibody H1H7814N N297Q described in the Examples below. In certain embodiments, BA is an anti-STEAP2 antibody comprising an HCVR according to SEQ ID NO:1 and an LCVR according to SEQ ID NO:5. In certain embodiments, BA is an N297Q antibody comprising an HCVR according to SEQ ID NO:1 and an LCVR according to SEQ ID NO:5. In certain embodiments, BA is an anti-STEAP2 antibody comprising one, two, three, four, five, or six of HCDR1, HCDR2, HCDR3, LCDR1, LCDR2, and LCDR3 according to SEQ ID NOS:2, 3, 4, 6, 7, and 8, respectively. In certain embodiments, BA is an N297Q antibody comprising one, two, three, four, five, or six of HCDR1, HCDR2, HCDR3, LCDR1, LCDR2, and LCDR3 according to SEQ ID NOS:2, 3, 4, 6, 7, and 8, respectively. N297Q indicates that one or more residues 297 are mutated from asparagine (N) to glutamine (Q). In certain embodiments, each residue 297 is mutated to Q. In certain embodiments, numbering is according to the EU numbering system. In certain embodiments of this paragraph, k is from one to four. In certain embodiments, k is one, two, three, or four. In certain embodiments, k is four.

In any of the embodiments above, BA is an anti-PRLR antibody. In certain embodiments, BA is the anti-PRLR antibody H1H6958N₂ described in the Examples below. In certain embodiments, BA is the anti-PRLR antibody H1H6958N₂ N297Q described in the Examples below. In certain embodiments, BA is an anti-PRLR antibody comprising an HCVR according to SEQ ID NO:9 and an LCVR according to SEQ ID NO:13. In certain embodiments, BA is an N297Q antibody comprising an HCVR according to SEQ ID NO:9 and an LCVR according to SEQ ID NO:13. In certain embodiments, BA is an anti-PRLR antibody comprising one, two, three, four, five, or six of HCDR1, HCDR2, HCDR3, LCDR1, LCDR2, and LCDR3 according to SEQ ID NOS:10, 11, 12, 14, 15, and 16, respectively. In certain embodiments, BA is an N297Q antibody comprising one, two, three, four, five, or six of HCDR1, HCDR2, HCDR3, LCDR1, LCDR2, and LCDR3 according to SEQ ID NOS:10, 11, 12, 14, 15, and 16, respectively. N297Q indicates that one or more residues 297 are mutated from asparagine (N) to glutamine (Q). In certain embodiments, each residue 297 is mutated to Q. In certain embodiments, numbering is according to the EU numbering system. In certain embodiments of this paragraph, k is from one to four. In certain embodiments, k is one, two, three, or four. In certain embodiments, k is four.

In any preceding embodiment in this section, R⁷ is —NR^{7a}R^{7b} wherein R^{7a} and R^{7b} are independently in each instance, hydrogen, alkyl, alkenyl, alkynyl, cycloalkyl, aryl, heteroaryl, acyl, or an amino acid residue, wherein alkyl, alkenyl, alkynyl, cycloalkyl, aryl, heteroaryl, and acyl are optionally substituted. In certain embodiments R^{7a} is hydrogen and R^{7b} is an amino acid residue.

Methods of Preparing Compounds or Payloads, and Linker-Payloads The compounds provided herein can be prepared, isolated, or obtained by any method apparent to those of skill in the art. Exemplary methods of preparation are described in detail in the Examples below.

In certain embodiments, provided herein are compounds (e.g., linker-payloads or linker-prodrug payloads) selected from the group consisting of

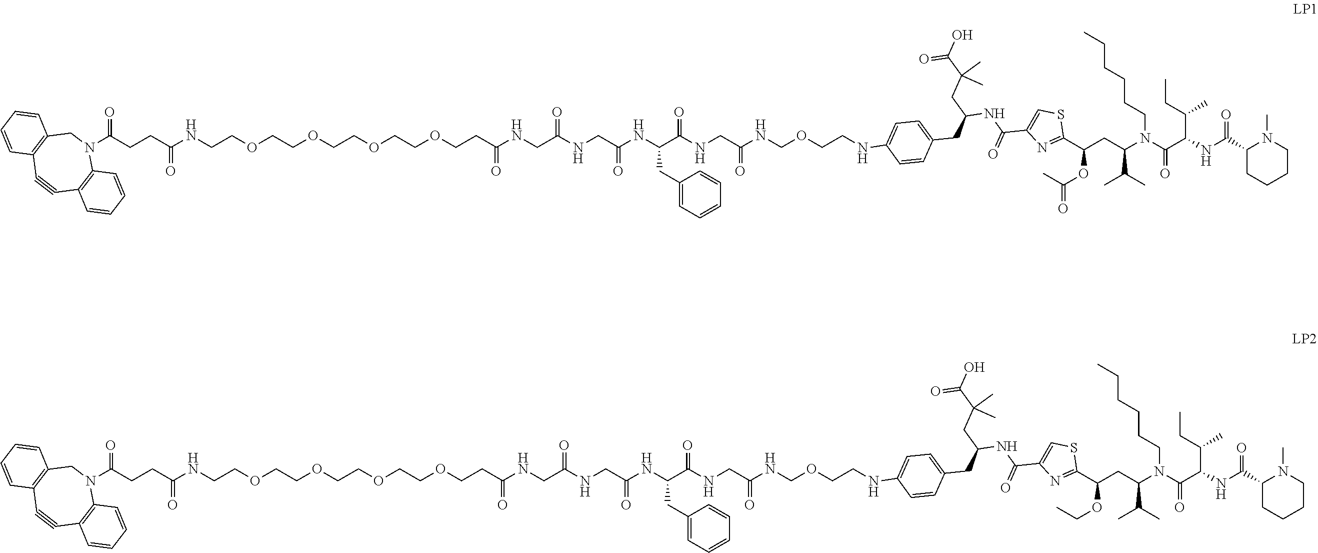 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCOCNC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C ; CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCOCNC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C]

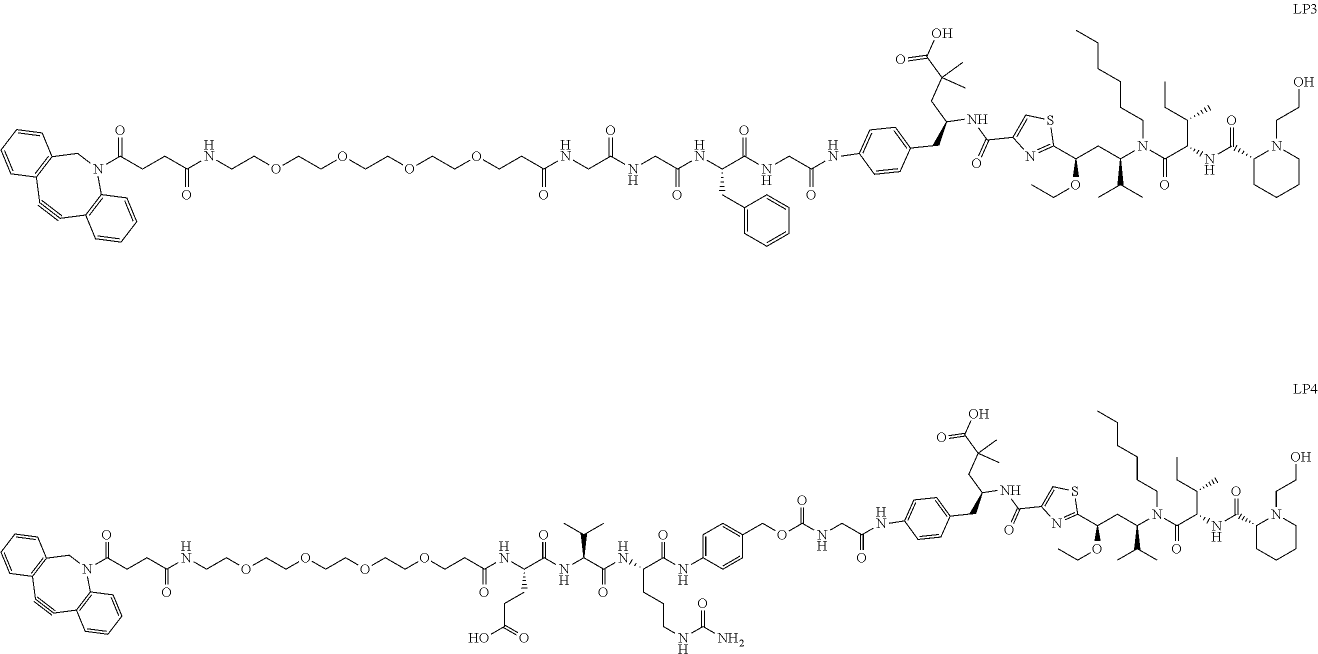 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C ; CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CNC(=O)OCc3ccc(NC(=O)[C@H](CCCNC(N)=O)NC(=O)[C@@H](NC(=O)[C@H](CCC(=O)O)NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C]

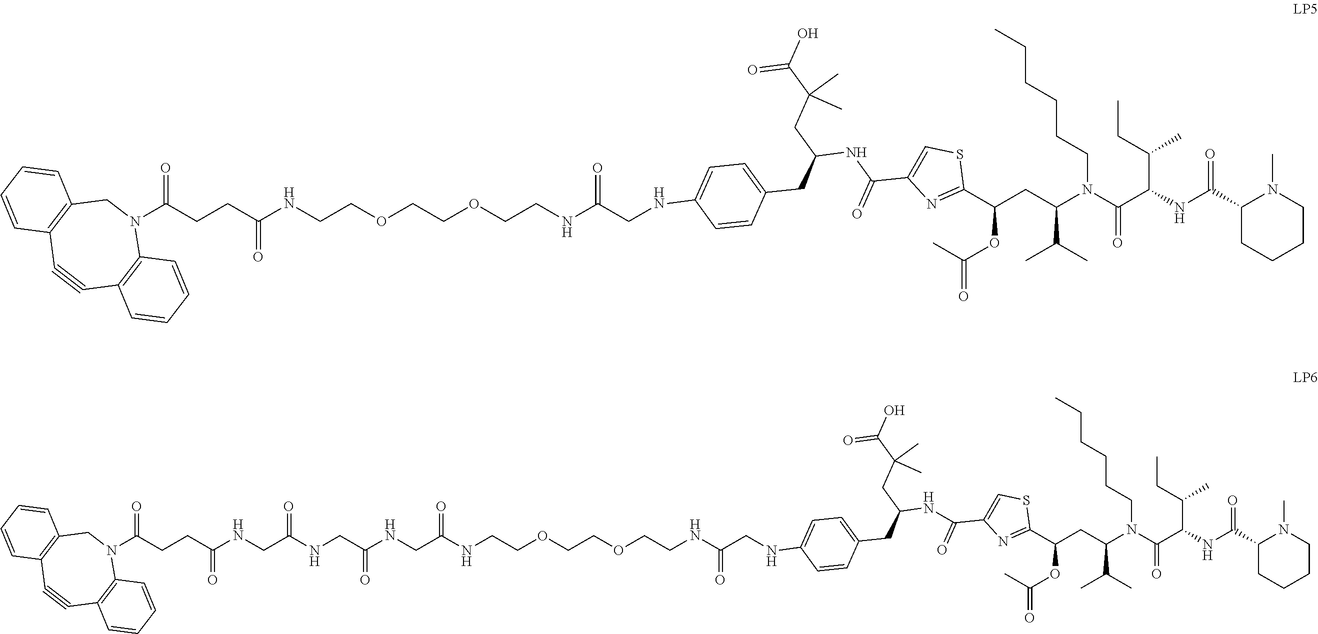 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCNC(=O)CCC(=O)N3Cc4ccccc4C#Cc4ccccc43)cc2)CC(C)(C)C(=O)O)cs1)C(C)C ; CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCNC(=O)CNC(=O)CNC(=O)CNC(=O)CCC(=O)N3Cc4ccccc4C#Cc4ccccc43)cc2)CC(C)(C)C(=O)O)cs1)C(C)C]

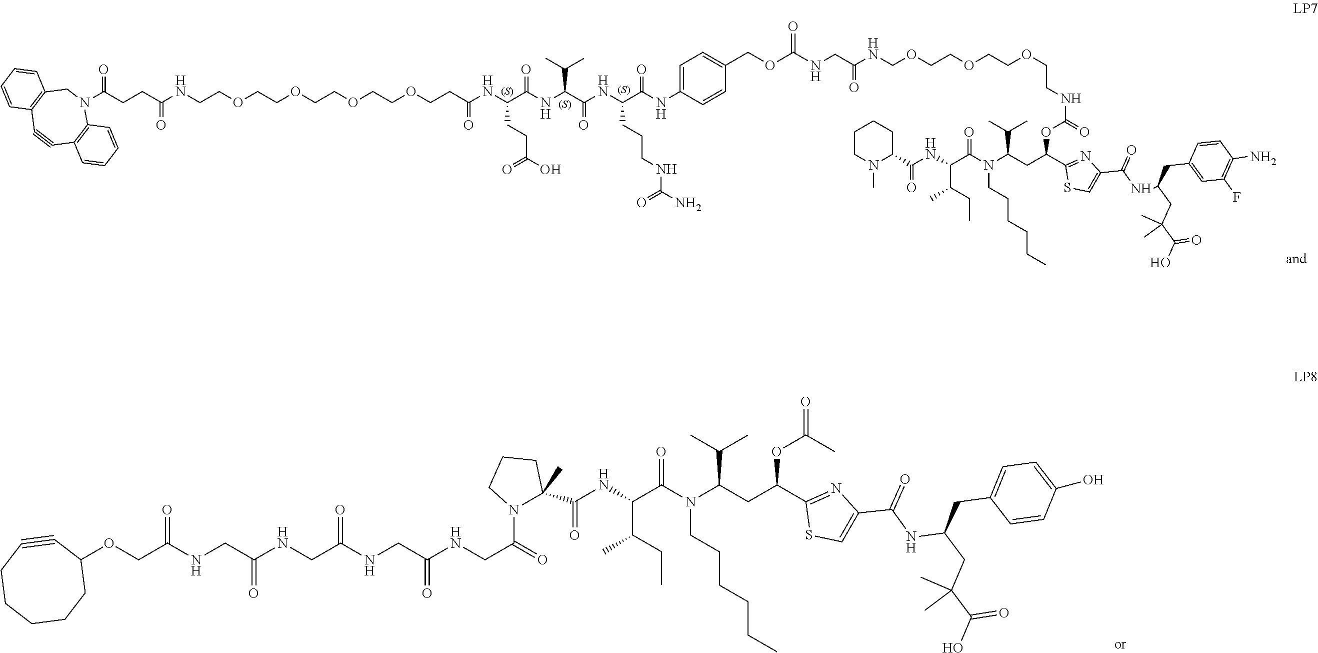

a pharmaceutically acceptable salt thereof. In certain embodiments within this paragraph, all diastereomers are contemplated. For example, in one embodiment, the stereochemistry within

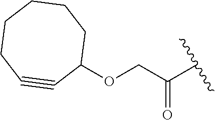 [SMILES: *C(=O)COC1C#CCCCCC1]

is undefined or racemic. By way of further example, in one embodiment, the stereochemistry within

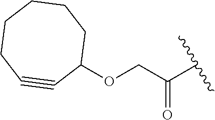 [SMILES: *C(=O)COC1C#CCCCCC1]

is (R)—. By way of further example, in one embodiment, the stereochemistry within

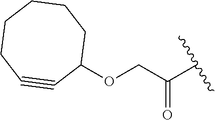 [SMILES: *C(=O)COC1C#CCCCCC1]

(S)—. By way of further example, in embodiment, the stereochemistry within

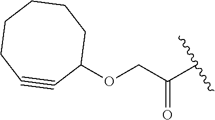 [SMILES: *C(=O)COC1C#CCCCCC1]

is (R)— in excess of (S)—. By way of further example, in one embodiment, the stereochemistry within

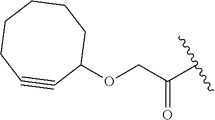 [SMILES: *C(=O)COC1C#CCCCCC1]

is (S)— in excess of (R)—.

In certain embodiments, provided herein are compounds (e.g., linker-payloads or linker-prodrug payloads) selected from the group consisting of

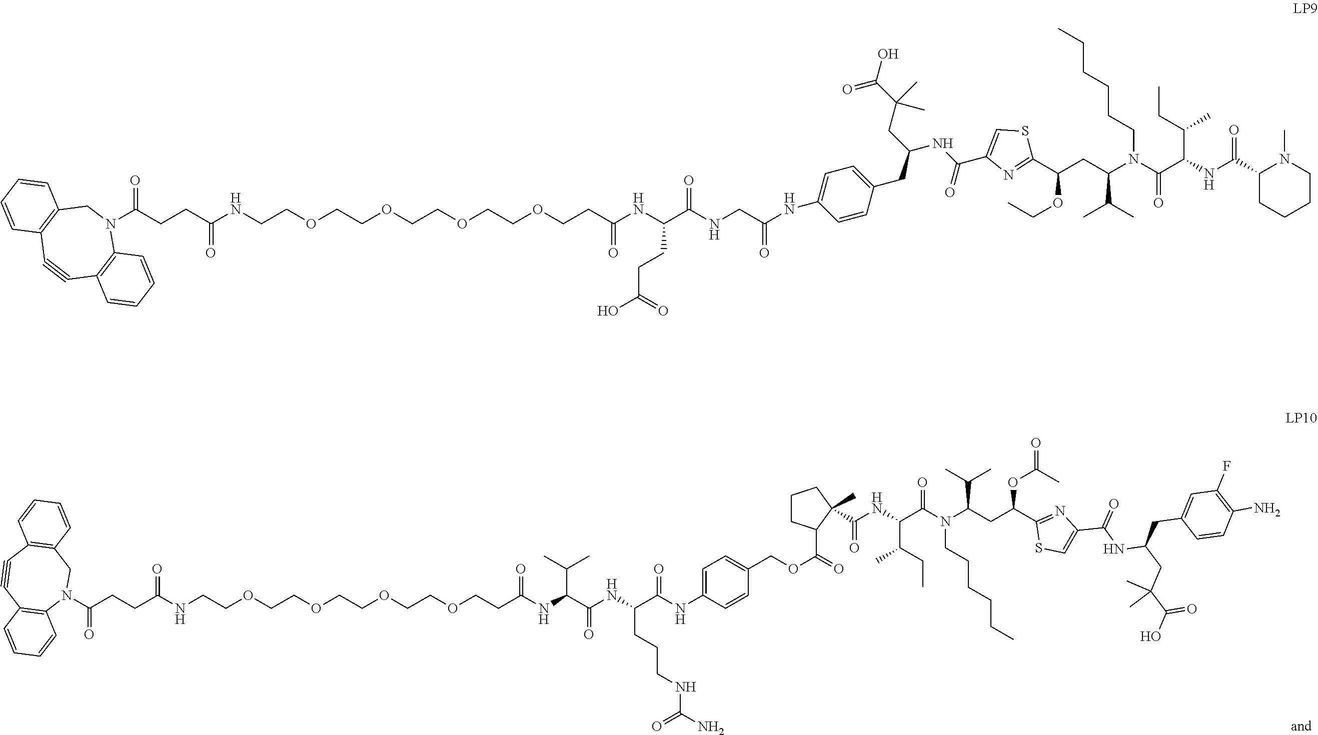 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CNC(=O)[C@H](CCC(=O)O)NC(=O)CCOCCOCCOCCOCCNC(=O)CCC(=O)N3Cc4ccccc4C#Cc4ccccc43)cc2)CC(C)(C)C(=O)O)cs1)C(C)C ; CCCCCCN(C(=O)[C@@H](NC(=O)[C@@]1(C)CCCC1C(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

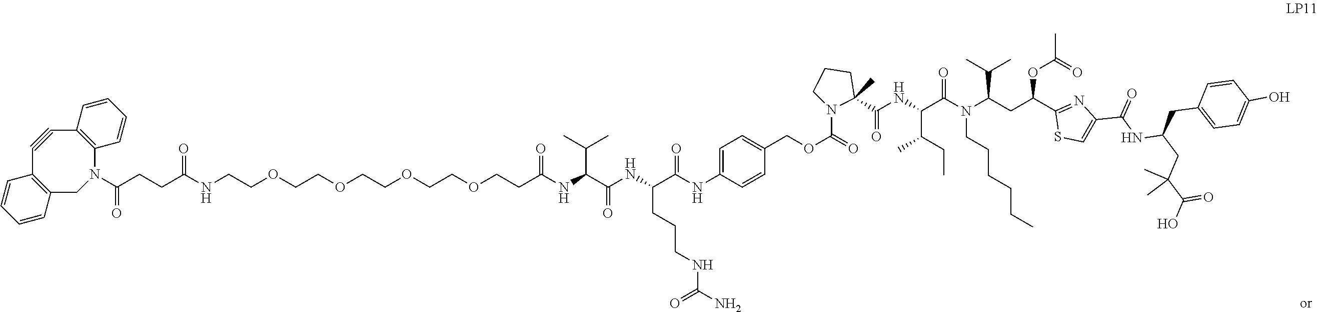 [SMILES: CCCCCCN(C(=O)[C@@H](NC(=O)[C@@]1(C)CCCN1C(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)[C@@H](C)CC)[C@H](C[C@@H](OC(C)=O)c1nc(C(=O)N[C@@H](Cc2ccc(O)cc2)CC(C)(C)C(=O)O)cs1)C(C)C]

a pharmaceutically acceptable salt thereof. In certain embodiments within this paragraph, all diastereomers are contemplated. For example, in one embodiment, the stereochemistry within

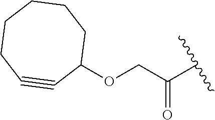 [SMILES: *C(=O)COC1C#CCCCCC1]

is undefined or racemic. By way of further example, in one embodiment, the stereochemistry within

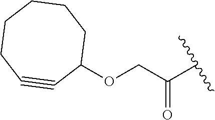 [SMILES: *C(=O)COC1C#CCCCCC1]

is (R)—. By way of further example, in one embodiment, the stereochemistry within

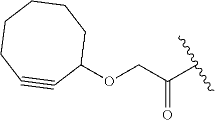 [SMILES: *C(=O)COC1C#CCCCCC1]

(S)—. By way of further example, in embodiment, the stereochemistry within

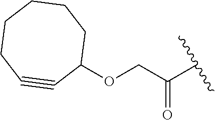 [SMILES: *C(=O)COC1C#CCCCCC1]

is (R)— in excess of (S)—. By way of further example, in one embodiment, the stereochemistry within

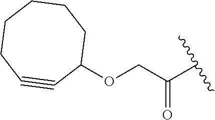 [SMILES: *C(=O)COC1C#CCCCCC1]

is (S)— in excess of (R)—.

The conjugates described herein can be synthesized by coupling the linker-payloads or linker-prodrug payloads described herein with a binding agent, for example, an antibody under standard conjugation conditions (see, e.g., Doronina et al. Nature Biotechnology 2003, 21, 778, which is incorporated herein by reference in its entirety). When the binding agent is an antibody, the antibody may be coupled to a linker-payload via one or more cysteine or lysine residues of the antibody. Linker-payloads can be coupled to cysteine residues, for example, by subjecting the antibody to a reducing agent, for example, dithiotheritol, to cleave the disulfide bonds of the antibody, purifying the reduced antibody, for example, by gel filtration, and subsequently treating the antibody with a linker-payload containing a suitable reactive moiety, for example, a maleimido group. Suitable solvents include, but are not limited to water, DMA, DMF, and DMSO. Linker-payloads or linker-prodrug payloads containing a reactive group, for example, an activated ester or acid halide group, can be coupled to lysine residues of the antibody. Suitable solvents include, but are not limited to, water, DMA, DMF, and DMSO. Conjugates can be purified using known protein techniques, including, for example, size exclusion chromatography, dialysis, and ultrafiltration/diafiltration.

Binding agents, for example antibodies, can also be conjugated via click chemistry reactions. In some embodiments of said click chemistry reactions, the linker-payload includes a reactive group, for example an alkyne, that is capable of undergoing a regioisomeric 1,3-cycloaddition reaction with an azide. Such suitable reactive groups are described above. The antibody includes one or more azide groups. Such antibodies include antibodies functionalized with, for example, azido-polyethylene glycol groups. In certain embodiments, such functionalized antibody is derived by treating an antibody having at least one glutamine residue, for example, heavy chain Gln295, with a primary amine compound in the presence of the enzyme transglutaminase (e.g., to generate a transglutaminase-modified antibody or antigen-binding fragment thereof). In certain embodiments, such functionalized or transglutaminase-modified antibody is derived by treating an antibody having at least one glutamine residue, for example, heavy chain Gln297, with a primary amine compound in the presence of the enzyme transglutaminase. Such antibodies include Asn297Gln (N297Q) mutants. In certain embodiments, such functionalized antibody is derived by treating an antibody having at least two glutamine residues, for example, heavy chain Gln295 and heavy chain Gln297, with a primary amine compound in the presence of the enzyme transglutaminase. Such antibodies include Asn297Gln (N297Q) mutants. In certain embodiments, the antibody has two heavy chains as described in this paragraph for a total of two or a total of four glutamine residues.

In certain embodiments, the antibody comprises two glutamine residues, one in each heavy chain. In particular embodiments, the antibody comprises a Q295 residue in each heavy chain. In further embodiments, the antibody comprises one, two, three, four, five, six, seven, eight, or more glutamine residues. These glutamine residues can be in heavy chains, light chains, or in both heavy chains and light chains. These glutamine residues can be wild-type residues, or engineered residues. The antibodies can be prepared according to standard techniques.

Those of skill will recognize that antibodies are often glycosylated at residue N297, near residue Q295 in a heavy chain sequence. Glycosylation at residue N297 can interfere with a transglutaminase at residue Q295 (see Dennler et al., supra). Accordingly, in particular embodiments, the antibody is not glycosylated. In certain embodiments, the antibody is deglycoslated or aglycosylated. In particular embodiments, an antibody heavy chain has an N297 mutation. Alternatively stated, the antibody is mutated to no longer have an asparagine residue at position 297. In particular embodiments, an antibody heavy chain has an N297Q mutation. Such an antibody can be prepared by site-directed mutagenesis to remove or disable a glycosylation sequence or by site-directed mutagenesis to insert a glutamine residue at a site apart from any interfering glycosylation site or any other interfering structure. Such an antibody also can be isolated from natural or artificial sources.

The antibody without interfering glycosylation is then reacted or treated with a primary amine compound. In certain embodiments, an aglycosylated antibody is reacted or treated with a primary amine compound to produce a glutaminyl-modified antibody or transglutaminase-modified antibody. In certain embodiments, a deglycosylated antibody is reacted or treated with a primary amine compound to produce a glutaminyl-modified antibody or transglutaminase-modified antibody.

The primary amine can be any primary amine that is capable of forming a covalent bond with a glutamine residue in the presence of a transglutaminase. Useful primary amines are described herein. The transglutaminase can be any transglutaminase deemed suitable by those of skill in the art. In certain embodiments, the transglutaminase is an enzyme that catalyzes the formation of an isopeptide bond between a free amine group on the primary amine compound and the acyl group on the side chain of a glutamine residue. Transglutaminase is also known as protein-glutamine-γ-glutamyltransferase. In particular embodiments, the transglutaminase is classified as EC 2.3.2.13. The transglutaminase can be from any source deemed suitable. In certain embodiments, the transglutaminase is microbial. Useful transglutaminases have been isolated from Streptomyces mobaraense, Streptomyces cinnamoneum, Streptomyces griseo-carneum, Streptomyces lavendulae, and Bacillus subtilis. Non-microbial transglutaminases, including mammalian transglutaminases, can also be used. In certain embodiments, the transglutaminase can be produced by any technique or obtained from any source deemed suitable by the practitioner of skill. In particular embodiments, the transglutaminase is obtained from a commercial source.

In particular embodiments, the primary amine compound comprises a reactive group capable of further reaction after transglutamination. In these embodiments, the glutaminyl-modified antibody or transglutaminase-modified antibody can be reacted or treated with a reactive payload or prodrug payload compound or a reactive linker-payload or linker-prodrug compound to form an antibody-payload conjugate or an antibody-linker-payload conjugate. In certain embodiments, the primary amine compound comprises an azide.

In certain embodiments, the glutaminyl-modified antibody or transglutaminase-modified antibody is reacted or treated with a reactive linker-payload to form an antibody-linker-payload conjugate. The reaction can proceed under conditions deemed suitable by those of skill in the art. In certain embodiments, the glutaminyl-modified antibody or transglutaminase-modified antibody is contacted with the reactive linker-payload or linker-prodrug payload compound under conditions suitable for forming a bond between the glutaminyl-modified antibody or transglutaminase-modified antibody and the linker-payload or linker-prodrug payload compound. Suitable reaction conditions are well known to those in the art. Exemplary reactions are provided in the Examples below.

### Pharmaceutical Compositions and Methods of Treatment

Provided herein are methods of treating and preventing diseases, conditions, or disorders comprising administering a therapeutically or prophylactically effective amount or one or more of the compounds disclosed herein, for example, one or more of the compounds of a formula provided herein. Diseases, disorders, and/or conditions include, but are not limited to, those associated with the antigens listed herein.

The compounds described herein can be administered alone or together with one or more additional therapeutic agents. The one or more additional therapeutic agents can be administered just prior to, concurrent with, or shortly after the administration of the compounds described herein. This disclosure also includes pharmaceutical compositions comprising any of the compounds described herein in combination with one or more additional therapeutic agents, and methods of treatment comprising administering such combinations to subjects in need thereof.

Suitable additional therapeutic agents include, but are not limited to, a second tubulysin, an autoimmune therapeutic agent, a hormone, a biologic, or a monoclonal antibody. Suitable therapeutic agents also include, but are not limited to any pharmaceutically acceptable salts, acids, or derivatives of a compound set forth herein.

In some embodiments of the methods described herein, multiple doses of a compound described herein (or a pharmaceutical composition comprising a combination of a compound described herein and any of the additional therapeutic agents mentioned herein) may be administered to a subject over a defined time course. The methods according to this embodiment of the disclosure comprise sequentially administering to a subject multiple doses of a compound described herein. As used herein, “sequentially administering” means that each dose of the compound is administered to the subject at a different point in time, for example, on different days separated by a predetermined interval (e.g., hours, days, weeks, or months). This disclosure includes methods which comprise sequentially administering to the patient a single initial dose of a compound described herein, followed by one or more secondary doses of the compound, and optionally followed by one or more tertiary doses of the compound.

The terms “initial dose,” “secondary doses,” and “tertiary doses,” refer to the temporal sequence of administration of the compounds described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all include the same amount the compound described herein, but generally can differ from one another in terms of frequency of administration. In certain embodiments, the amount of the compound included in the initial, secondary and/or tertiary doses varies from one another (e.g., adjusted up or down as appropriate) during the course of treatment. In certain embodiments, two or more (e.g., two, three, four, or five) doses are administered at the beginning of the treatment regimen as “loading doses” followed by subsequent doses that are administered on a less frequent basis (e.g., “maintenance doses”).

In certain exemplary embodiments of this disclosure, each secondary and/or tertiary dose is administered one to twenty-six (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose the compound which is administered to a patient prior to the administration of the very next dose in the sequence with no intervening doses.

The methods according to this embodiment of the disclosure may comprise administering to a patient any number of secondary and/or tertiary doses of the compound. For example, in certain embodiments, only a single secondary dose is administered to the patient. In other embodiments, two or more (e.g., two, three, four, five, six, seven, eight, or more) secondary doses are administered to the patient. Likewise, in certain embodiments, only a single tertiary dose is administered to the patient. In other embodiments, two or more (e.g., two, three, four, five, six, seven, eight, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient one to two weeks or one to two months after the immediately preceding dose. Similarly, in embodiments involving multiple tertiary doses, each tertiary dose may be administered at the same frequency as the other tertiary doses. For example, each tertiary dose may be administered to the patient two to twelve weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

This disclosure includes administration regimens in which two to six loading doses are administered to a patient at a first frequency (e.g., once a week, once every two weeks, once every three weeks, once a month, once every two months, etc.), followed by administration of two or more maintenance doses to the patient on a less frequent basis. For example, according to this embodiment of the disclosure, if the loading doses are administered at a frequency of once a month, then the maintenance doses may be administered to the patient once every six weeks, once every two months, once every three months, etc.

This disclosure includes pharmaceutical compositions of the compounds and/or conjugates described herein, for example, the compounds Formulae I, Ia, Iaa, II, III, IV, V, VI, and/or conjugates thereof, LP9, LP10, LP11, BA-1, BA-2, BA-3, BA-4, BA-5, and BA-6, for example, compositions comprising a compound described herein, a salt, stereoisomer, regioisomer, polymorph thereof, and a pharmaceutically acceptable carrier, diluent, and/or excipient. Examples of suitable carriers, diluents and excipients include, but are not limited to, buffers for maintenance of proper composition pH (e.g., citrate buffers, succinate buffers, acetate buffers, phosphate buffers, lactate buffers, oxalate buffers, and the like), carrier proteins (e.g., human serum albumin), saline, polyols (e.g., trehalose, sucrose, xylitol, sorbitol, and the like), surfactants (e.g., polysorbate 20, polysorbate 80, polyoxolate, and the like), antimicrobials, and antioxidants.

In some examples, set forth herein is a method of treating cancer comprising administering to a patient having said cancer a therapeutically effective amount of a compound of Formulae I, II, III, IV, V, and VI, and/or conjugates thereof, or a pharmaceutical composition thereof. In some embodiments, provided herein is a method of treating cancer comprising administering to a patient having said cancer a therapeutically effective amount of an antibody-tubulysin conjugate described herein, or a pharmaceutical composition thereof. In some embodiments, the binding agent, for example, the antibody, of the conjugates, for example, antibody-drug conjugates described herein interact with or bind to tumor antigens, including antigens specific for a type of tumor or antigens that are shared, overexpressed, or modified on a particular type of tumor. Examples include, but are not limited to, alpha-actinin-4 with lung cancer, ARTC1 with melanoma, BCR-ABL fusion protein with chronic myeloid leukemia, B-RAF, CLPP or Cdc27 with melanoma, CASP-8 with squamous cell carcinoma, and hsp70-2 with renal cell carcinoma as well as the following shared tumor-specific antigens, for example, BAGE-1, GAGE, GnTV, KK-LC-1, MAGE-A2, NA88-A, and TRP2-INT2. Further examples of tumor antigens include, but are not limited to, PSMA, PRLR, MUC16, HER2, EGFRvIII, anti-STEAP2, and MET.

The compounds disclosed herein can be used for treating primary and/or metastatic tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the compounds provided herein are used to treat one or more of the following cancers renal cell carcinoma, pancreatic carcinoma, head and neck cancer (e.g., head and neck squamous cell carcinoma [HNSCC]), prostate cancer, castrate-resistant prostrate cancer, malignant gliomas, osteosarcoma, colorectal cancer, gastric cancer (e.g., gastric cancer with MET amplification), mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer wherein “residual cancer” means the existence or persistence of one or more cancerous cells in a subject following treatment with an anti-cancer therapy, and Wilms' tumor. In some embodiments, the cancer is breast cancer. In some embodiments, the cancer is prostate cancer. In one embodiments, provided is a method for treating tumors that express an antigen selected from the group consisting of PRLR and STEAP2 including administering to the subject an effective treatment amount of a pharmaceutical composition comprising a compound having the following formula

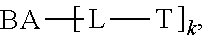

as described elsewhere herein.

In some examples, set forth herein is a method of preventing prostate cancer comprising administering to a patient having said disorder a prophylactically effective amount of a compound of Formulae I, Ia, Iaa, II, III, IV, V, VI, and/or conjugates thereof, LP9, LP10, LP11, BA-1, BA-2, BA-3, BA-4, BA-5, and BA-6, or a pharmaceutical composition thereof.

### Examples

Provided herein are novel tubulysins, protein conjugates thereof, and methods for treating diseases, disorders, and conditions including administering the tubulysins and conjugates.

Certain embodiments of this disclosure are illustrated by the following non-limiting examples. As used herein, the symbols and conventions used in these processes, schemes, and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the Examples, and throughout the specification.

| Abbreviation | Term or Phrase |
|---|---|
| ADC | Antibody-drug conjugate |
| Aglycosylated antibody | Antibody that does not have any glycan |
| API | Atmospheric pressure ionization |
| Aq. | Aqueous |
| Boc | tert-butoxycarbonyl |
| BupH | Thermo Scientific Prod# 28372, containing 100 mM sodium |
| | phosphate and 150 mM sodium chloride, potassium free, pH was |
| | adjusted from 7.2 to 7.6-7.8 MQ, unless otherwise noted. |
| CD | Cyclodextrin |
| COT | Cyclooctynol |
| CTRL | Antibody isotype control |
| Da | Dalton |
| DAD | Diode array detector |
| DAR | Drug to antibody ratio |
| DCM | Dichloromethane |
| DIBAC | 11,12-didehydro-5,6-dihydro-Dibenz[b,f]azocine |
| DIBAC-Suc | 11,12-didehydro-5,6-dihydro-Dibenz[b,f]azocine succinamic acid |
| DIBAC-Suc-PEG4- | {4-[(2S)-2-[(2S)-2-[1-(4-{2-azatricyclo[10.4.0.04,9]hexadeca- |
| VC-pAB-PNP | 1(12),4(9),5,7,13,15-hexaen-10-yn-2-yl}-4-oxobutanamido)- |
| | 3,6,9,12-tetraoxapentadecan-15-amido]-3-methylbutanamido]- |
| | 5-(carbamoylamino)pentanamido]phenyl}methyl 4-nitrophenyl |
| | carbonate |
| DIBACT | 3H-Benzo[c]-1,2,3-triazolo[4,5-e][1]benzazocine, 8,9-dihydro- |
| DIC | Diisopropylcarbodiimide |
| DIPEA | Diisopropylethylamine |
| DMF | N,N-dimethylformamide |
| DMSO | Dimethylsulfoxide |
| EC | Enzyme commission |
| ELSD | Evaporative light scattering detector |
| ESI | Electrospray ionization |
| Fmoc | N-(9-fluorenylmethyloxycarbonyl) |
| Fmoc-vcPAB-PNP | N-Fmoc-L-valine-L-citrulline-p-aminobenzyl alcohol p-nitrophenyl carbonate |
| g | Gram |
| HATU | 2-(7-Aza-1H-benzotriazole-1-yl)-1,1,3,3-tetramethyluronium hexafluorophosphate |
| HC | Heavy chain of immunoglobulin |
| HEK | Human embryonic kidney (cells) |
| HPLC | High performance liquid chromatography |
| hr, h, or hrs | Hours |
| LC | Light chain of immunoglobulin |
| LCh | Liquid chromatography |
| MALDI | Matrix-assisted laser desorption/ionization |
| MC | Maleimidocaproyl |
| mg | milligrams |
| min | minutes |
| mL | milliliters |
| mmh | myc-myc-hexahistidine tag |
| AL | microliters |
| mM | millimolar |
| UM | micromolar |
| MMAE | Monomethyl auristatin E |
| MS | Mass spectrometry |
| MsC1 | Methanesulfonyl chloride |
| MSD | Mass-selective detector |
| MTG | Microbial transglutaminase (MTG EC 2.3.2.13, Zedira, Darmstadt, Germany) |
| MW | Molecular weight |
| ncADC | Non-cytotoxic antibody drug conjugate |
| NHS | N-hydroxysuccinimide |
| nM | nanomolar |
| NMR | Nuclear magnetic resonance |
| PABC | Para-aminobenzyloxy(carbonyl) |
| PBS | 10 mM sodium phosphate buffer and 150 mM sodium chloride |
| PBSg | 10 mM phosphate, 150 mM sodium chloride, 5% glycerol |
| PEG | Polyethyleneglycol |
| PNP | p-nitrophenyl |
| MC-VC-PAB-PNP | Maleimidocaproyl-L-valine-L-citrulline-p-aminobenzyl alcohol p-nitrophenyl carbonate |
| ppm | Parts per million (chemical shift, δ) |
| RP | Reversed phase |
| rt | room temperature |
| SDS-PAGE | Sodium dodecylsulfate polyacrylamide gel electrophoresis |
| SEC | Size exclusion chromatography |
| Suc | Succinic acid |
| TCEP | Tris(2-carboxyethyl)phosphine hydrochloride |
| TEA | Triethylamine |
| TMS | tetramethylsilane |
| TFA | Trifluoroacetic acid |
| TG | Transglutaminase |
| THF | Tetrahydrofuran |
| TOF | Time-of-flight |
| TRSQ | Trastuzumab N297Q |
| UPLC | Ultra Performance Liquid Chromatography |
| UV | Ultraviolet |
| VA | Valine-alanine |
| VC | Valine-citrulline |
| VC-PABC | Valine-citrulline-para-aminobenzyloxy(carbonyl) |
| ZP3A | Azido-PEG₃-NH₂ or a residue thereof |

Reagents and solvents were obtained from commercial sources such as Sinopharm Chemical Reagent Co. (SCRC), Sigma-Aldrich, Alfa, or other vendors, unless explicitly stated otherwise. ¹H NMR and other NMR spectra can be recorded on a Bruker AVIII 400 or Bruker AVIII 500. The data were processed with Nuts software or MestReNova software, measuring proton shifts in parts per million (ppm) downfield from an internal standard, for example, tetramethylsilane (TMS).

HPLC-MS measurements were run on an Agilent 1200 HPLC/6100 SQ System using the following conditions: Method A for HPLC-MS measurements included, as the Mobile Phase: A: Water (0.01% trifluoroacetic acid (TFA)), B: acetonitrile (0.01% TFA); Gradient Phase: 5% of B increased to 95% of B within 15 min; Flow Rate: 1.0 mL/min; Column: SunFire C18, 4.6×50 mm, 3.5 μm; Column Temperature: 50° C. Detectors: Analog to Digital Converter (ADC) Evaporative Light-scattering Detector (ELSD or ADC ELSD), Diode array detector (DAD) (214 nm and 254 nm), electrospray ionization-atmospheric ionization (ES-API). Method B for HPLC-MS measurements included, as the Mobile Phase: A: Water (10 mM NH₄HCO₃), B: acetonitrile; Gradient Phase: 5% increased to 95% of B within 15 min; Flow Rate: 1.0 mL/min; Column:) (Bridge C18, 4.6×50 mm, 3.5 μm; Column Temperature: 50° C. Detectors: ADC ELSD, DAD (214 nm and 254 nm), mass selective detector (MSD) (ES-API).

LC-MS measurements were run on an Agilent 1200 HPLC/6100 SQ System using the following conditions: Method A for LC-MS measurements included, as the Instrument: WATERS 2767; column: Shimadzu Shim-Pack, PRC-ODS, 20×250 mm, 15 μm, two connected in series; Mobile Phase: A: Water (0.01% TFA), B: acetonitrile (0.01% TFA); Gradient Phase: 5% of B increased to 95% of B within 3 min; Flow Rate: 1.8-2.3 mL/min; Column: SunFire C18, 4.6×50 mm, 3.5 μm; Column Temperature: 50° C. Detectors: ADC ELSD, DAD (214 nm and 254 nm), MSD ES-API. Method B for LC-MS measurement included, as the Instrument: Gilson GX-281; column: Xbridge Prep C18 10 μm OBD, 19×250 mm; Mobile Phase: A: Water (10 mM NH₄HCO₃), B: Acetonitrile; Gradient Phase: 5% increased to 95% of B within 3 min; Flow Rate: 1.8-2.3 mL/min; Column: XBridge C18, 4.6×50 mm, 3.5 μm; Column Temperature: 50° C. Detectors: ADC ELSD, DAD (214 nm and 254 nm), MSD ES-API.

Preparative high-pressure liquid chromatography (Prep-HPLC) in an acidic or basic solvent system was on a Gilson GX-281 instrument. The acidic solvent system included a Waters SunFire 10 μm C18 column (100 Å, 250×19 mm), and solvent A for prep-HPLC was water/0.05% TFA and solvent B is acetonitrile (Method A). The elution conditions were a linear gradient increase of solvent B from 5% to 100% over a time period of 20 min at a flow rate of 30 mL/min. The basic solvent system included a Waters Xbridge 10 μm C18 column (100 Å, 250×19 mm), and solvent A for prep-HPLC was water/10 mM ammonium bicarbonate (NH₄HCO₃) and solvent B is acetonitrile (Method B). The elution conditions were a linear gradient increase of solvent B from 5% to 100% over a time period of 20 min at a flow rate of 30 mL/min.

Flash chromatography was performed on a Biotage instrument, with Agela Flash Column silica-CS cartridges; Reversed phase flash chromatography was performed on Biotage instrument, with Boston ODS or Agela C18 cartridges unless explicitly stated otherwise.

cLogP was calculated based on JChemFunctions.

Analytical chiral HPLC method—SFC conditions
a) Instrument: SFC Method Station (Thar, Waters)
b) Column: CHIRALPAK AD-H/AS-H/OJ-H/OD-H 4.6×100 mm, 5 μm (Daicel)
c) Column temperature: 40° C.
d) Mobile phase: CO₂/IPA (0.1% DEA)=55/45
e) Flow: 4.0 mL/min
f) Back Pressure: 120 Bar
g) Injection volume: 2

Preparative Chiral HPLC Method—SFC Conditions
a) Instrument: SFC-80 (Thar, Waters)
b) Column: CHIRALPAK AD-H/AS-H/OJ-H/OD-H 20×250 mm, 10 μm (Daicel)
c) Column temperature: 35° C.
d) Mobile phase: CO₂/IPA (0.2% Methanol Ammonia)=30/70
e) Flow rate: 80 g/min
f) Back pressure: 100 bar
g) Detection wavelength: 214 nm
h) Cycle time: 6.0 min
i) Sample solution: 1500 mg dissolved in 70 mL Methanol
j) Injection volume: 2 mL (loading: 42.86 mg/injection)

### Preparation Methods

### Intermediates: MEP

Intermediates MEPa-c were commercially available. CAS numbers and structures appear below.

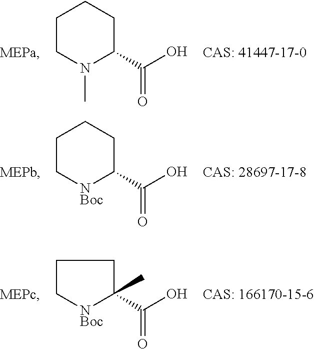

### Synthesis of ethyl 2-((1R,3R)-3-((2S,3S)-2-amino-N-hexyl-3-methylpentanamido)-1-ethoxy-4-methylpentyl)thiazole-4-carboxylate (1B)

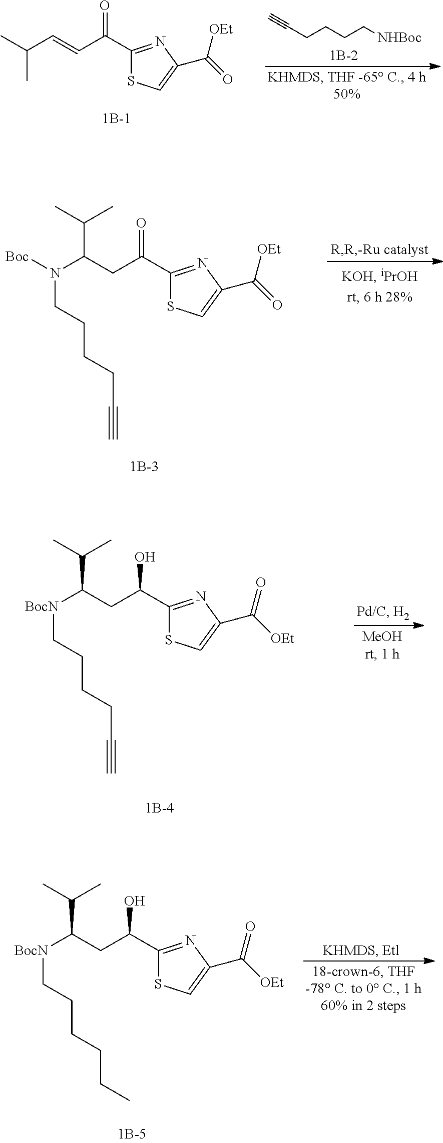 [SMILES: CCOC(=O)c1csc(C(=O)/C=C/C(C)C)n1 ; C#CCCCCNC(=O)OC(C)(C)C ; C#CCCCCN(C(=O)OC(C)(C)C)C(CC(=O)c1nc(C(=O)OCC)cs1)C(C)C ; C#CCCCCN(C(=O)OC(C)(C)C)[C@H](C[C@@H](O)c1nc(C(=O)OCC)cs1)C(C)C ; CCCCCCN(C(=O)OC(C)(C)C)[C@H](C[C@@H](O)c1nc(C(=O)OCC)cs1)C(C)C]

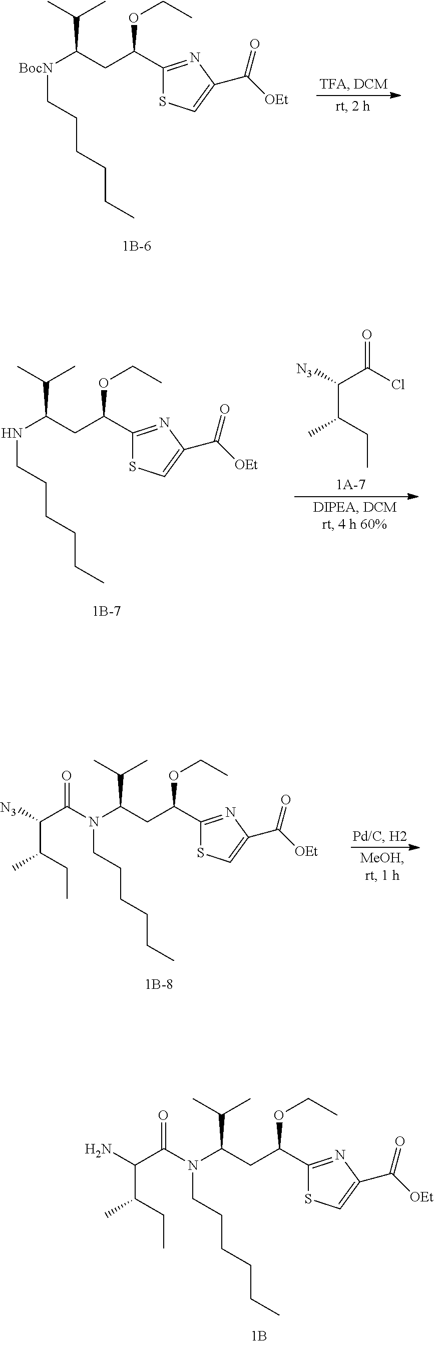

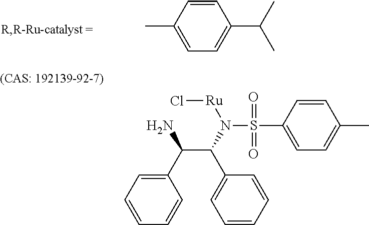

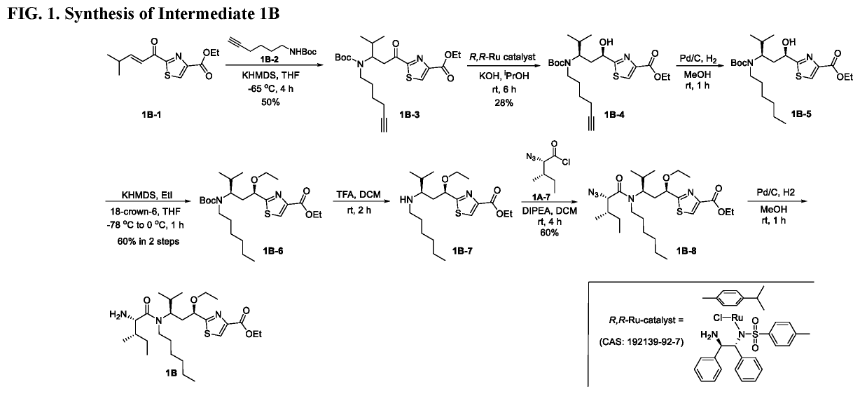

Intermediate 1B was synthesized as in FIG. 1.

Compound 1B-1 was synthesized according to WO 2008/138561 A1.

### Ethyl 2-(3-{[(tert-butoxy)carbonyl](hex-5-yn-1-yl)amino}-4-methylpentanoyl)-1,3-thiazole-4-carboxylate (1B-3)

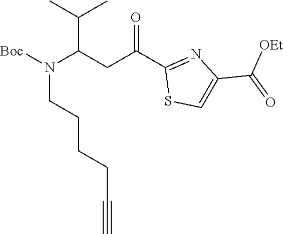 [SMILES: C#CCCCCN(C(=O)OC(C)(C)C)C(CC(=O)c1nc(C(=O)OCC)cs1)C(C)C]

To a −65° C. solution of compound 1B-2 (73 g, 0.37 mol) in dry THF (1.2 L) was subsequently added dropwise KHMDS (1 M in THF, 0.37 L, 0.37 mol) over thirty minutes followed by a solution of compound 1B-1 (62 g, 0.25 mol) in THF (0.20 L) over thirty minutes keeping the temperature below −60° C. The reaction mixture was stirred at −65° C. for four hours until 1B-1 was totally consumed, according to thin layer chromatography (TLC). The resulting mixture is quenched with sat. aq. ammonium chloride (0.30 L). The aqueous layer was extracted with ethyl acetate (0.5 L×3). All the organics were combined and washed with brine (0.5 L), dried over anhydrous sodium sulfate, filtered, and concentrated in vacuo. The residue was purified by silica gel column chromatography (10% ethyl acetate in petroleum ether) to give compound 1B-3 (55 g, 50% yield) as a yellow oil. ESI m/z: 351 (M-Boc+H)⁺. ¹H NMR (400 MHz, CDCl₃) δ 8.42 (s, 1H), 4.44 (q, J=7.2 Hz, 2H), 4.09 (br s, 1H), 3.70-3.42 (m, 2H), 3.30-2.99 (m, 2H), 2.25-2.15 (m, 2H), 2.12-1.90 (m, 2H), 1.70-1.55 (m, 2H), 1.55-1.43 (m, 5H), 1.42 (s, 9H), 1.00 (d, J=6.6 Hz, 3H), 0.93 (d, J=6.6 Hz, 3H) ppm.

### Ethyl 2-[(1R,3R)-3-{[(tert-butoxy)carbonyl](hex-5-yn-1-yl)amino}-1-hydroxy-4-methylpentyl]-1,3-thiazole-4-carboxylate (1B-4)

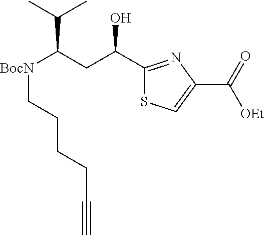 [SMILES: C#CCCCCN(C(=O)OC(C)(C)C)[C@H](C[C@@H](O)c1nc(C(=O)OCC)cs1)C(C)C]

To a solution of compound 1B-3 (54 g, 0.12 mol) in isopropanol (0.60 L) was added R,R-Ru-catalyst (CAS: 192139-92-7, 3.9 g, 6.0 mmol) and potassium hydroxide (0.73 g, 12 mmol). The reaction was stirred at room temperature for six hours until 1B-3 was totally consumed according to TLC. The reaction mixture was quenched with sat. aq. Ammonium chloride (0.3 L). The mixture was extracted with ethyl acetate (0.5 L×3) and the combined organic extracts were washed with brine (0.5 L), dried over anhydrous sodium sulfate, filtered, and concentrated in vacuo. The crude product was purified by silica gel column chromatography (10-20% ethyl acetate in petroleum ether) to give compound 1B-4 (15 g, 28% yield) as yellow oil. ESI m/z: 453 (M+H)⁺, 475 (M+Na)⁺.

### Ethyl 2-[(1R,3R)-3-{[(tert-butoxy)carbonyl](hexyl)amino}-1-hydroxy-4-methylpentyl]-1,3-thiazole-4-carboxylate (1B-5)

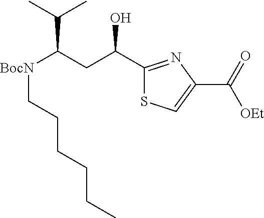 [SMILES: CCCCCCN(C(=O)OC(C)(C)C)[C@H](C[C@@H](O)c1nc(C(=O)OCC)cs1)C(C)C]

To a solution of compound 1B-4 (0.45 g, 1.0 mmol) in methanol (10 mL) was added 10% Palladium on carbon (50 mg, 11 wt %) under nitrogen. The suspension was degassed and purged with hydrogen three times, and was then stirred at room temperature under a hydrogen balloon for an hour. The reaction was a monitored by LCMS. The resulting suspension was filtered through Celite and the filtrate was concentrated in vacuo to give crude product 1B-5 (0.45 g, crude) as a white solid. Crude 1B-5 was used in the next step without further purification. ESI m/z: 457 (M+H)⁺, 479 (M+Na)⁺.

### Ethyl 2-[(1R,3R)-3-{[(tert-butoxy)carbonyl](hexyl)amino}-1-ethoxy-4-methylpentyl]-1,3-thiazole-4-carboxylate (1B-6)

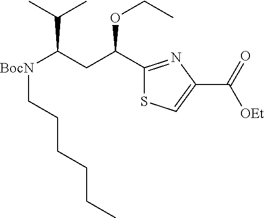 [SMILES: CCCCCCN(C(=O)OC(C)(C)C)[C@H](C[C@@H](OCC)c1nc(C(=O)OCC)cs1)C(C)C]

To a solution of compound 1B-5 (0.44 g, 1.0 mmol) and 18-crown-6 (0.53 g, 2.0 mmol) in THF (10 mL) was added a solution of KHMDS in THF (1.0 M, 2.0 mL, 2.0 mmol) dropwise over five minutes at −78° C. under nitrogen. The reaction mixture was stirred at −78° C. for thirty minutes before the addition of ethyliodide (0.78 g, 5.0 mmol). The mixture was slowly warmed to room temperature, stirred for an hour, and monitored by LCMS. The reaction mixture was cooled to −10° C., and the resulting mixture was quenched via water (20 mL) and extracted with ethyl acetate (20 mL×3). The combined organic solution was washed with brine (20 mL), dried over anhydrous sodium sulfate, filtered, and concentrated in vacuo. The crude product was purified by prep-HPLC (5-95% acetonitrile in aq. Ammonium bicarbonate (10 mM)) to give compound 1B-6 (0.29 g, 60% yield over two steps) as a white solid. ESI m/z: 485 (M+H), 507 (M+Na)⁺.

### Ethyl 2-[(1R,3R)-1-ethoxy-3-(hexylamino)-4-methylpentyl]-1,3-thiazole-4-carboxylate (1B-7)

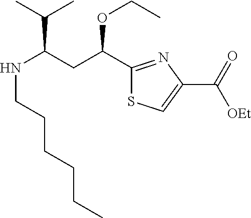 [SMILES: CCCCCCN[C@H](C[C@@H](OCC)c1nc(C(=O)OCC)cs1)C(C)C]

To a solution of compound 1B-6 (0.20 g, 0.41 mmol) in DCM (5.0 mL) was added TFA (1.0 mL) dropwise at room temperature. The mixture was stirred at room temperature for two hours until Boc was totally consumed as monitored by LCMS. The volatiles were removed in vacuo to provide crude product 1B-7 (0.12 g, crude) as a white solid. Crude 1B-7 was used in the next step without further purification. ESI m/z: 385 (M+H)⁺.

### Ethyl 2-[(1R,3R)-3-[(2S,3S)-2-azido-N-hexyl-3-methylpentanamido]-1-ethoxy-4-methylpentyl]-1,3-thiazole-4-carboxylate (1B-8)

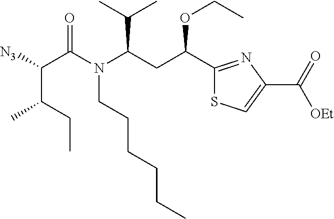 [SMILES: CCCCCCN(C(=O)[C@@H](N=[N+]=[N-])[C@@H](C)CC)[C@H](C[C@@H](OCC)c1nc(C(=O)OCC)cs1)C(C)C]

Compound 1B-8 was prepared as shown in FIG. 1 (0.12 g, 60% yield) as a white solid. ESI m/z: 520 (M+H)⁺, 542 (M+Na)⁺.

### Ethyl 2-[(1R,3R)-3-[(2S,3S)-2-amino-N-hexyl-3-methylpentanamido]-1-ethoxy-4-methylpentyl]-1,3-thiazole-4-carboxylate (1B)

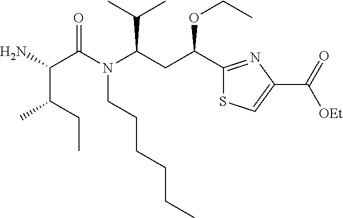 [SMILES: CCCCCCN(C(=O)[C@@H](N)[C@@H](C)CC)[C@H](C[C@@H](OCC)c1nc(C(=O)OCC)cs1)C(C)C]

To a solution of compound 1B-8 (0.10 g, 0.19 mmol) in methanol (10 mL) was added 10% Palladium on carbon (50 mg, 50 wt %) under nitrogen. The suspension was degassed and purged with hydrogen three times. The reaction was then stirred at room temperature under a hydrogen balloon for an hour, and monitored by LCMS. The resulting suspension was filtered through Celite and the filtrate was concentrated in vacuo to give intermediate 1B (0.16 g, 85% yield) as a white solid. Intermediate 1B was used in the next step without purification. ESI m/z: 498 (M+H)⁺.

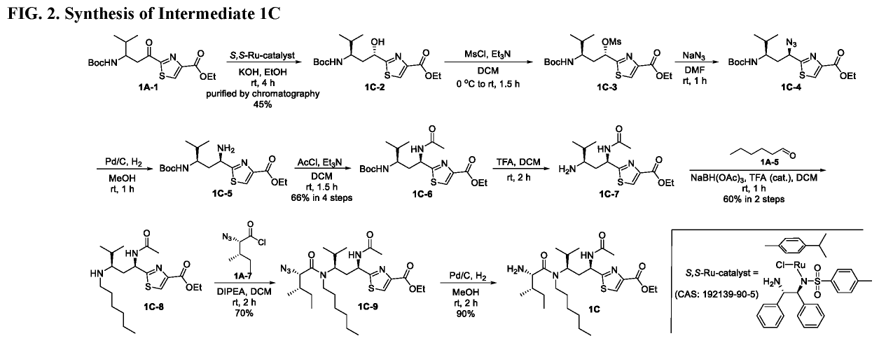

Intermediate 1C was synthesized as in FIG. 2.

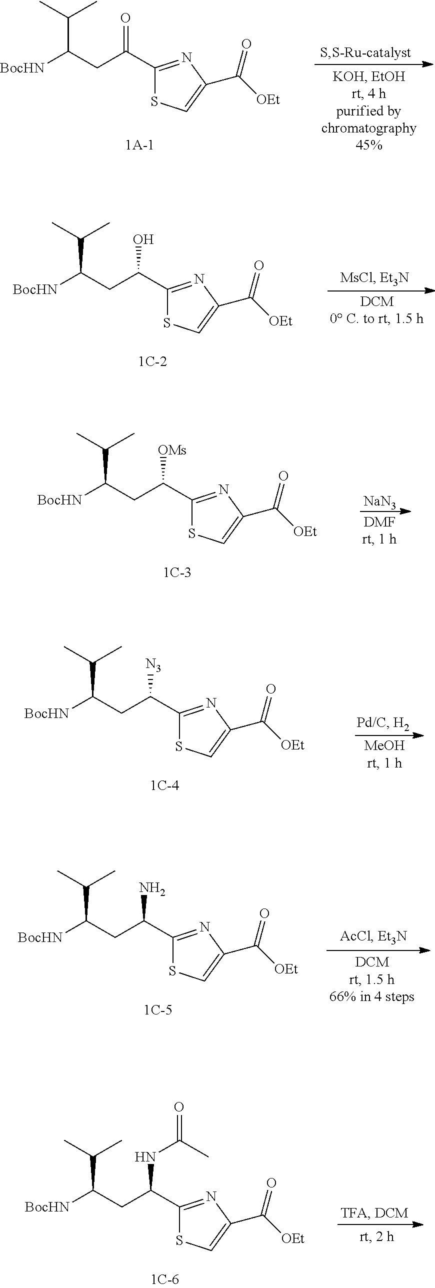

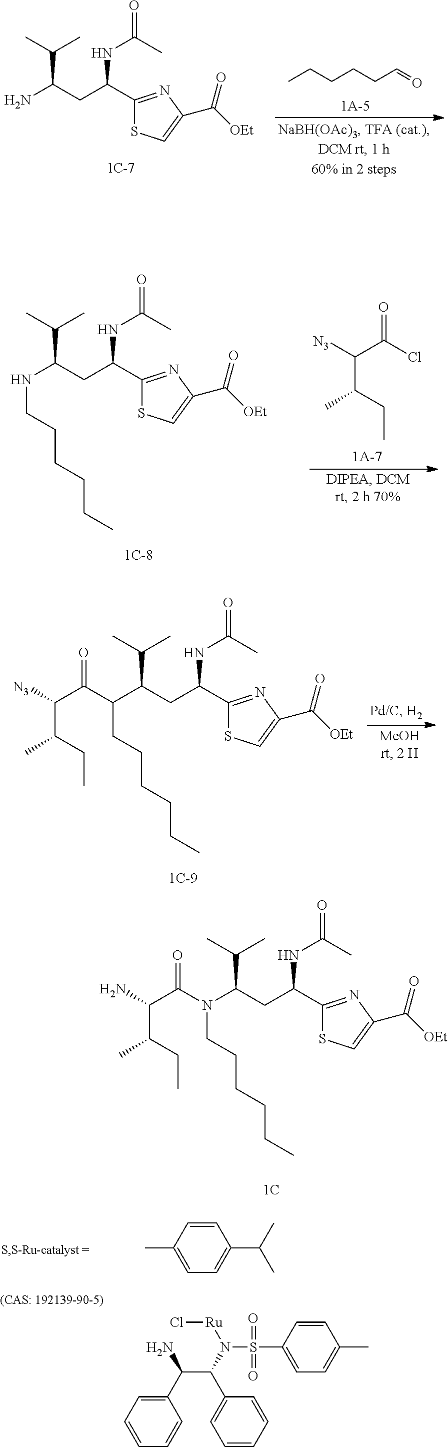

### Ethyl 2-[(1S,3R)-3-{[(tert-butoxy)carbonyl]amino}-1-hydroxy-4-methylpentyl]-1,3-thiazole-4-carboxylate (1C-2)

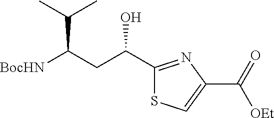 [SMILES: CCOC(=O)c1csc([C@@H](O)C[C@@H](NC(=O)OC(C)(C)C)C(C)C)n1]

Compound 1C-2 (1.7 g, 45% yield, 80e.e %.) was prepared as a colorless oil as shown in the above scheme. ESI m/z: 373 (M+H)⁺. TLC (silica gel): R_{f}=0.3 (33% ethyl acetate in petroleum ether; the R_{f} value for the other diastereoisomer is 0.4).

A small amount of the product was separated by chiral-HPLC (Column: R′R WHELK mm, 10 μm (Daicel), Mobile phase: CO₂/MeOH (0.2% methanol ammonia)=90/10) to give enantiopure product 1C-2 (>99.9% ee). Chiral HPLC: >99.9% using an AS, AD, OD, and OJ column. ¹H NMR (400 MHz, CDCl₃) δ 8.42 (s, 1H), 6.53 (d, J=9.3 Hz, 1H), 6.25 (d, J=4.7 Hz, 1H), 4.81 (d, J=4.8 Hz, 1H), 4.30-4.27 (m, 2H), 3.53 (s, 1H), 2.06-1.89 (m, 1H), 1.77-1.70 (m, 2H), 1.34 (s, 9H), 1.30 (t, J=7.2 Hz, 3H), 0.81 (d, J=3.4 Hz, 3H), 0.78 (d, J=3.4 Hz, 3H) ppm.

### Ethyl 2-[(1S,3R)-3-{[(tert-butoxy)carbonyl]amino}-1-(methanesulfonyloxy)-4-methylpentyl]-1,3-thiazole-4-carboxylate (1C-3)

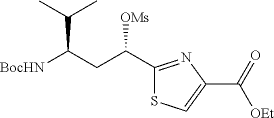 [SMILES: CCOC(=O)c1csc([C@H](C[C@@H](NC(=O)OC(C)(C)C)C(C)C)OS(C)(=O)=O)n1]

To a suspension of compound 1C-2 (1.4 g, 4.0 mmol, 80% ee) in DCM (50 mL) was subsequently added triethylamine (0.60 g, 6.0 mmol) and methanesulfonyl chloride (0.55 g, 4.8 mmol) dropwise at 0° C. After the reaction turned clear, the reaction mixture was stirred at 0° C. for an hour, then at room temperature for thirty minutes, and was monitored by TLC. The solution was successively washed with aq. Hydrochloride (1 N, 50 mL), water (50 mL), aq. Sodium carbonate (10%, 50 mL), and brine (50 mL). The resulting organic solution was dried over anhydrous sodium sulfate, filtered, and concentrated in vacuo to give crude compound 1C-3 (1.6 g, crude) as a yellow oil. Crude 1C-3 was used in the next step without further purification. ESI m/z: 451 (M+H)⁺.

### Ethyl 2-[(1R,3R)-1-azido-3-{[(tert-butoxy)carbonyl]amino}-4-methylpentyl]-1,3-thiazole-4-carboxylate (1C-4)

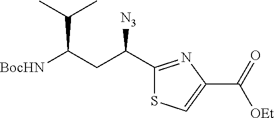 [SMILES: CCOC(=O)c1csc([C@@H](C[C@@H](NC(=O)OC(C)(C)C)C(C)C)N=[N+]=[N-])n1]

To a stirred mixture of compound 1C-3 (1.6 g, crude) in DMF (10 mL) was added sodium azide (1.2 g, 18 mmol) at room temperature. The reaction mixture was stirred at room temperature for an hour, and monitored by LCMS. The mixture was then diluted with water (50 mL) and extracted with ethyl acetate (50 mL×3). The combined organic solution was washed with water (50 mL) and brine (50 mL), dried over anhydrous sodium sulfate, filtered, and concentrated in vacuo to give crude compound 1C-4 (1.3 g, crude) as a yellow oil. ESI m/z: 398 (M+H)⁺.

### Ethyl 2-[(1R,3R)-1-amino-3-{[(tert-butoxy)carbonyl]amino}-4-methylpentyl]-1,3-thiazole-4-carboxylate (1C-5)

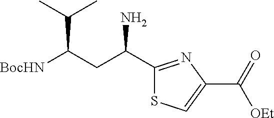 [SMILES: CCOC(=O)c1csc([C@H](N)C[C@@H](NC(=O)OC(C)(C)C)C(C)C)n1]

To a solution of compound 1C-4 (1.3 g, crude) in methanol (50 mL) was added 10% Palladium on carbon (0.12 g, 10 wt %) under nitrogen. The suspension was degassed and purged with hydrogen three times. The reaction is then stirred at room temperature under a hydrogen balloon for an hour, and monitored by LCMS. The resulting suspension was filtered through Celite and the filtrate was concentrated in vacuo to give crude compound 1C-5 (1.0 g, crude) as a yellow oil. Crude 1C-5 was used in the next step without further purification. ESI m/z: 371 (M+H)⁺.

### Ethyl 2-[(1R,3R)-3-{[(tert-butoxy)carbonyl]amino}-1-acetamido-4-methylpentyl]-1,3-thiazole-4-carboxylate (1C-6)

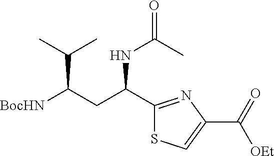 [SMILES: CCOC(=O)c1csc([C@@H](C[C@@H](NC(=O)OC(C)(C)C)C(C)C)NC(C)=O)n1]

To a stirred suspension of compound 1C-5 (1.0 g, crude) in DCM (50 mL) was subsequently added triethylamine (0.45 g, 4.5 mmol) and acetylchloride (0.28 g, 3.6 mmol) at 0° C. After the reaction turned clear, the reaction mixture was stirred at room temperature for 1.5 hours, and monitored by LCMS. The resulting solution was then washed with aq. Hydrochloride (1 N, 50 mL), water (50 mL), aq. Sodium carbonate (10%, 50 mL), brine (50 mL), dried over anhydrous sodium sulfate, filtered, and concentrated in vacuo. The residue was purified by silica gel column chromatography (15-20% ethyl acetate in petroleum ether) to give compound 1C-6 (1.0 g, 66% yield over four steps) as a yellow oil. ESI m/z: 413 (M+H)⁺.

### Ethyl 2-[(1R,3R)-3-amino-1-acetamido-4-methylpentyl]-1,3-thiazole-4-carboxylate (1C-7)

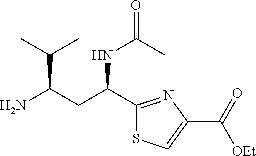 [SMILES: CCOC(=O)c1csc([C@@H](C[C@@H](N)C(C)C)NC(C)=O)n1]

To a solution of compound 1C-6 (1.3 g, 3.0 mmol) in DCM (20 mL) was added TFA (4 mL) at 0° C. The mixture was stirred at room temperature for an hour, and monitored by LCMS. The volatiles were removed in vacuo to give crude compound 1C-7 (1.0 g, crude) as a yellow solid. Crude 1C-7 was used in the next step without further purification. ESI m/z: 314 (M+H)⁺.

### Ethyl 2-[(1R,3R)-1-acetamido-3-(hexylamino)-4-methylpentyl]-1,3-thiazole-4-carboxylate (1C-8)

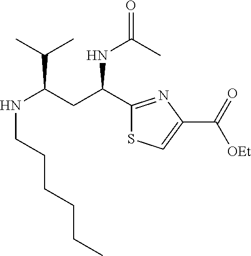 [SMILES: CCCCCCN[C@H](C[C@@H](NC(C)=O)c1nc(C(=O)OCC)cs1)C(C)C]

To a solution of crude compound 1C-7 (0.70 g, 2.2 mmol) in DCM (30 mL) under nitrogen was subsequently added hexanal (1A-5, 0.26 g, 2.6 mmol) dropwise over five minutes, then sodium triacetoxyborohydride (0.70 g, 3.3 mmol), and two drops of TFA. The reaction mixture was stirred at room temperature for an hour, and monitored by LCMS. The resulting mixture was washed with water (20 mL), aq. sodium carbonate (10%, 20 mL), brine (20 mL), dried over anhydrous sodium sulfate, filtered, and concentrated in vacuo. The residue was purified by chiral-HPLC (Column: IG 20*250 mm, 10 μm, Mobile phase: CO₂/methanol (0.2% methanol ammonia)=80/20) to give compound 1C-8 (0.52 g, 60% yield in 2 steps) as a colorless oil. ESI m/z: 398 (M+H)⁺. ¹H NMR (400 MHz, DMSO-d₆) δ 8.77 (d, J=7.8 Hz, 1H), 8.39 (s, 1H), 5.33-5.26 (m, 1H), 4.38-4.18 (m, 2H), 2.56-2.50 (m, 1H), 2.39-2.30 (m, 2H), 1.89 (s, 3H), 1.83-1.70 (m, 2H), 1.37-1.19 (m, 12H), 0.85-0.79 (m, 9H) ppm. >99.9% ee using IG columns.

### Ethyl 2-[(1R,3R)-3-[(2S,3S)-2-azido-N-hexyl-3-methylpentanamido]-1-acetamido-4-methylpentyl]-1,3-thiazole-4-carboxylate (1C-9)

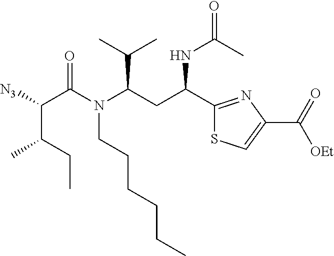 [SMILES: CCCCCCN(C(=O)[C@@H](N=[N+]=[N-])[C@@H](C)CC)[C@H](C[C@@H](NC(C)=O)c1nc(C(=O)OCC)cs1)C(C)C]

To a mixture of compound 1C-8 (0.20 g, 0.50 mmol) in DCM (5 mL) was subsequently added DIPEA (0.13 g, 1.0 mmol) and compound 1A-7 (0.18 g, 1.0 mmol). The mixture was stirred at room temperature for two hours and monitored by LCMS. The volatiles were removed in vacuo and the residue was purified by silica gel column chromatography (15-20% ethyl acetate in petroleum ether) to give compound 1C-9 (0.19 g, 70% yield) as a yellow oil. ESI m/z: 537 (M+H)⁺.

### Ethyl 2-[(1R,3R)-3-[(2S,3S)-2-amino-N-hexyl-3-methylpentanamido]-1-acetamido-4-methylpentyl]-1,3-thiazole-4-carboxylate (1C)

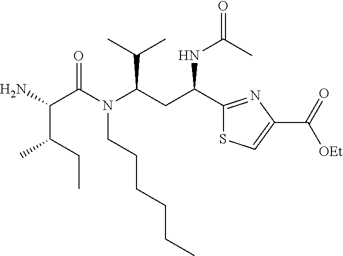 [SMILES: CCCCCCN(C(=O)[C@@H](N)[C@@H](C)CC)[C@H](C[C@@H](NC(C)=O)c1nc(C(=O)OCC)cs1)C(C)C]

To a solution of compound 1C-9 (0.19 g, 0.35 mmol) in methanol (10 mL) was added 10% Palladium on carbon (20 mg, 10 wt %) under nitrogen. The suspension was degassed and purged with hydrogen three times. The reaction was then stirred at room temperature under a hydrogen balloon for two hours, and monitored by LCMS. The resulting suspension was filtered through Celite and the filtrate was concentrated in vacuo. The residue was purified by silica gel column chromatography (50% ethyl acetate in petroleum ether) to give intermediate 1C (0.15 g, 90% yield) as a yellow oil. ESI m/z: 511 (M+H)⁺.

Compound C1-5 was prepared from intermediate 1B-4 following similar procedures as described in the preparation of Compound 1B as shown in the scheme below.

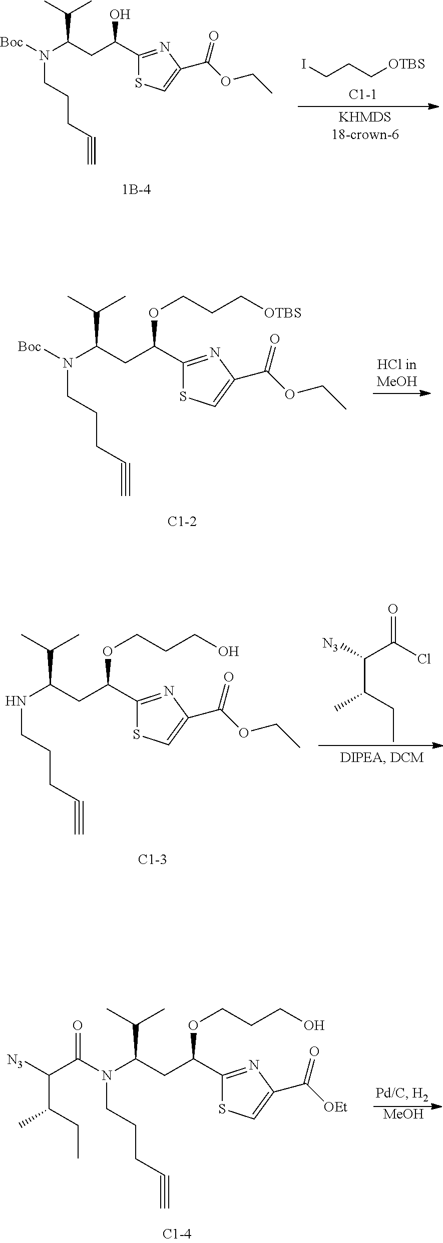

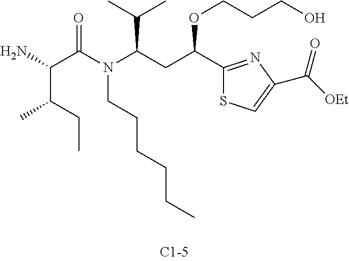 [SMILES: CCCCCCN(C(=O)[C@@H](N)[C@@H](C)CC)[C@H](C[C@@H](OCCCO)c1nc(C(=O)OCC)cs1)C(C)C]

### Intermediates: TUP

Intermediates TUP-3b-TUP-6b, TUPa, TUPb, TUPg, and TUPk were synthesized as described in International Patent Application No. PCT/US2021/038781, filed Jun. 23, 2021.

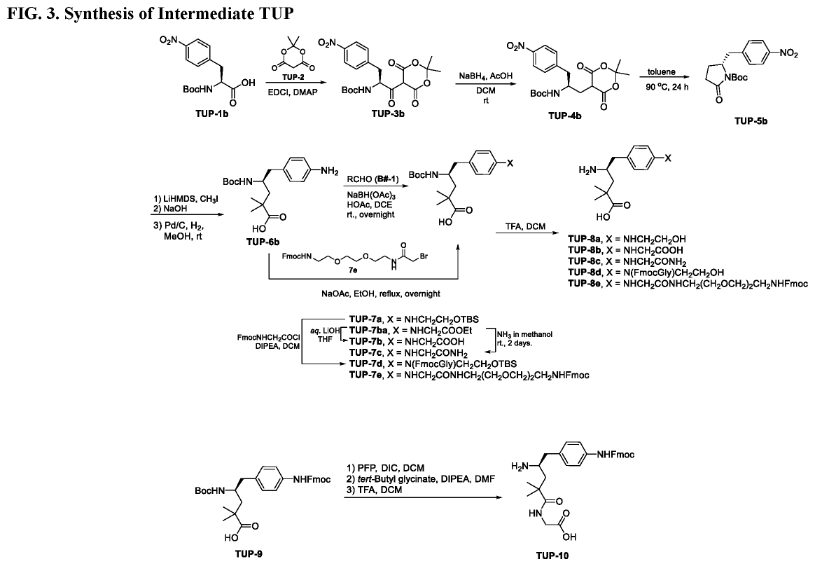

Synthesis of intermediates TUP-8a-TUP-8e and TUP-10 are described and below and in FIG. 3.

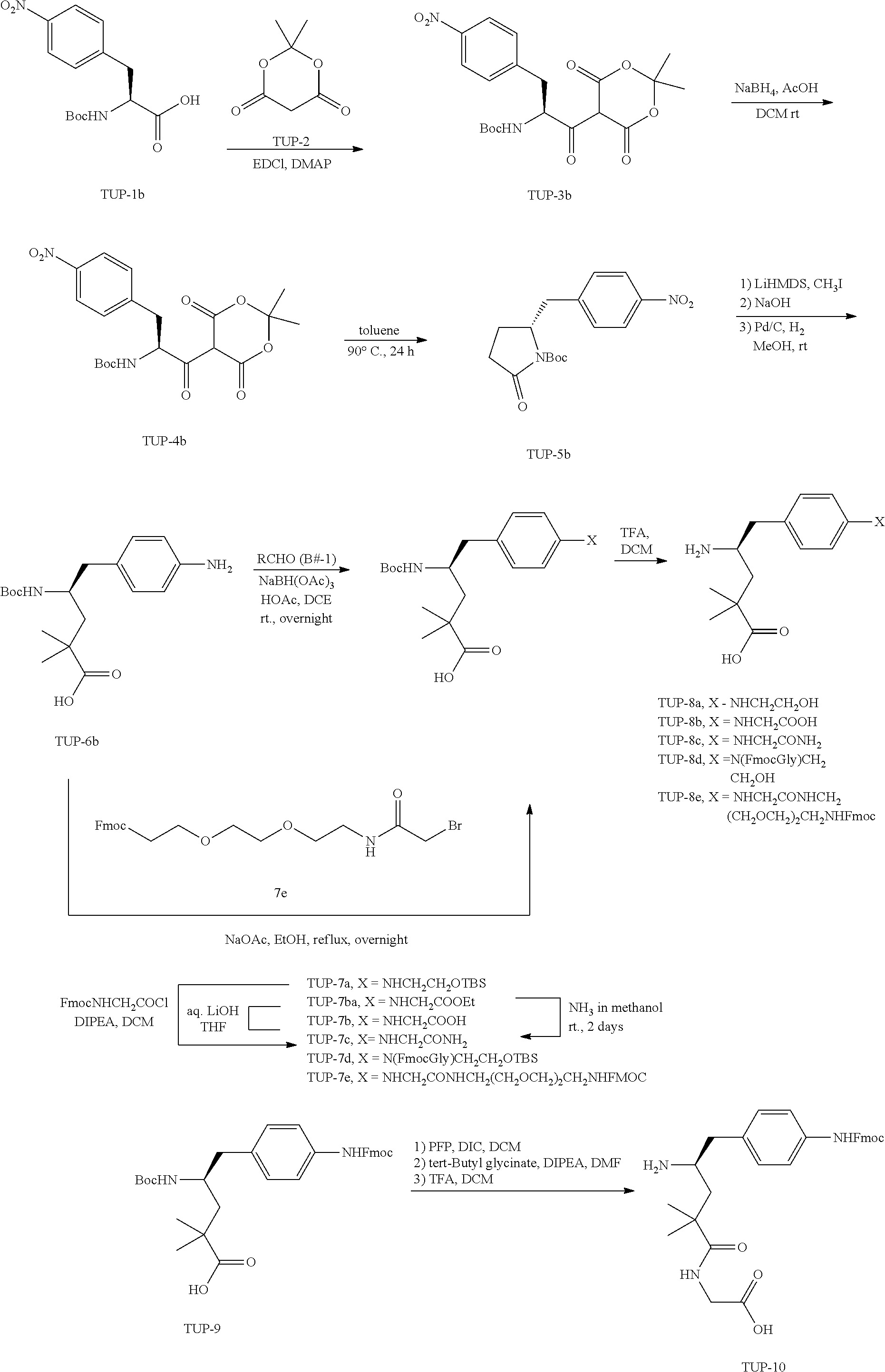

TABLE 1
Tubulysin Payloads Modified on Mep

| Pay- | | | | | |
|---|---|---|---|---|---|
| load | | | | | ESI |
| No. | Structures | cLogP | MF | MW | m/z |
| PA1 | 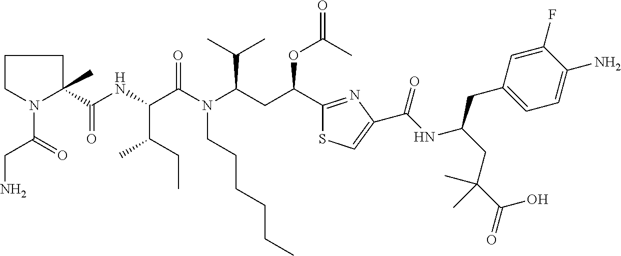 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 3.14 | C₄₅H₇₀- FN₇O₈S | 888.15 | 888.5 (M + H) |
| PA2 | 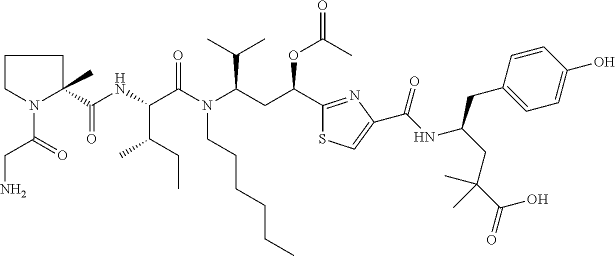 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(O)cc2)CC(C)(C)C(=O)O)cs1)C(C)C] | 3.52 | C₄₅H₇₀- N₆O₉S | 871.15 | 871.5 (M + H) |
| PA3 | 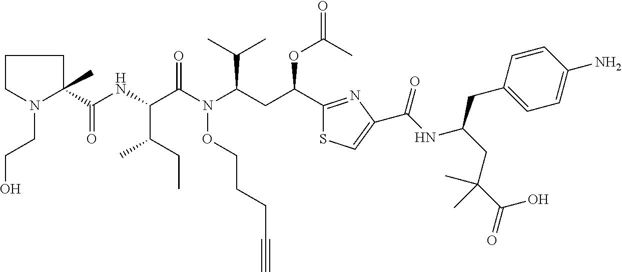 [SMILES: C#CCCCON(C(=O)[C@@H](NC(=O)[C@@]1(C)CCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 2.49 | C₄₄H₆₆- N₆O₉S | 855.11 | 855.4 (M + H) |
| PA4 | 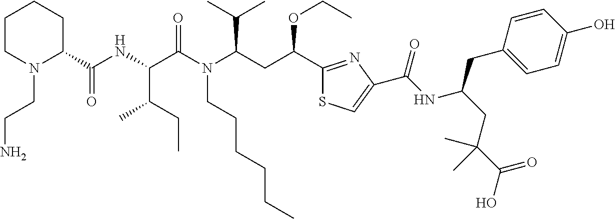 [SMILES: CCCCCCN(C(=O)[C@@H](NC(=O)[C@H]1CCCCN1CCN)[C@@H](C)CC)[C@H](C[C@@H](OCC)c1nc(C(=O)N[C@@H](Cc2ccc(O)cc2)CC(C)(C)C(=O)O)cs1)C(C)C] | 4.99 | C₄₅H₇₄- N₆O₇S | 843.18 | 843.3 (M + H) |
| PA5 | 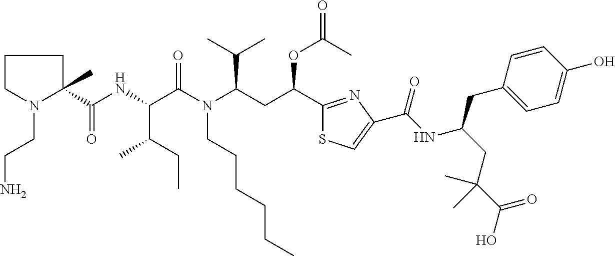 [SMILES: CCCCCCN(C(=O)[C@@H](NC(=O)[C@@]1(C)CCCN1CCN)[C@@H](C)CC)[C@H](C[C@@H](OC(C)=O)c1nc(C(=O)N[C@@H](Cc2ccc(O)cc2)CC(C)(C)C(=O)O)cs1)C(C)C] | 4.41 | C₄₅H₇₂- N₆O₈S | 857.17 | 857.3 (M + H) |
| PA6 | 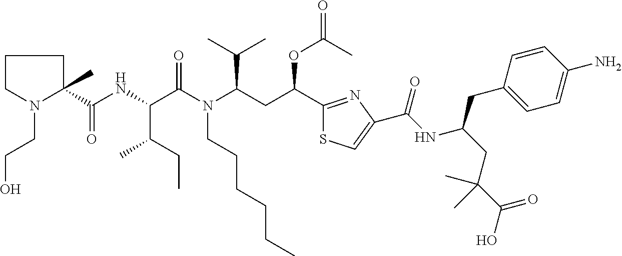 [SMILES: CCCCCCN(C(=O)[C@@H](NC(=O)[C@@]1(C)CCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 3.59 | C₄₅H₇₂- N₆O₈S | 857.17 | 857.3 (M + H) |
| PA7 | 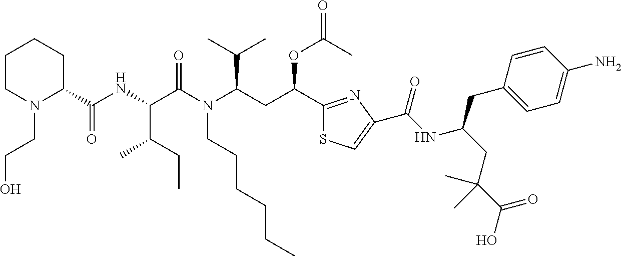 [SMILES: CCCCCCN(C(=O)[C@@H](NC(=O)[C@H]1CCCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 3.69 | C₄₅H₇₂- N₆O₈S | 857.17 | 857.3 (M + H) |
| PA8 | 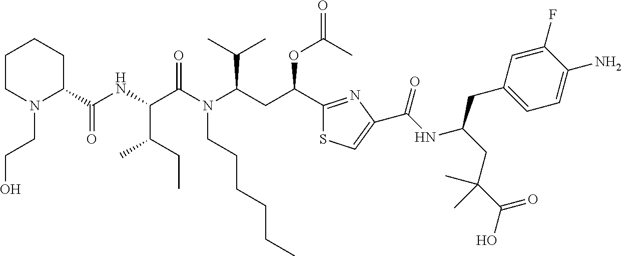 [SMILES: CCCCCCN(C(=O)[C@@H](NC(=O)[C@H]1CCCCN1CCO)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 3.83 | C₄₅H₇₁F- N₆O₈S | 875.16 | 875.4 (M + H) |
| PA9 | 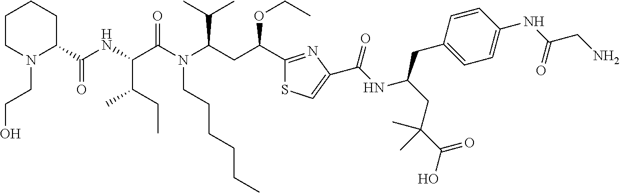 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] | 3.43 | C₄₇H₇₇- N₇O₈S | 900.23 | 450.9 (M/2 + H) |
| PA10 | 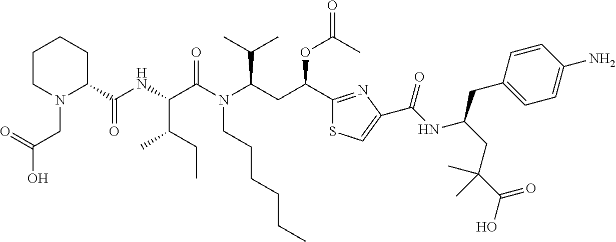 [SMILES: CCCCCCN(C(=O)[C@@H](NC(=O)[C@H]1CCCCN1CC(=O)O)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 3.80 | C₄₅H₇₀- N₆O₉S | 871.15 | 436.3 (M/2 + H) |
| PA11 | 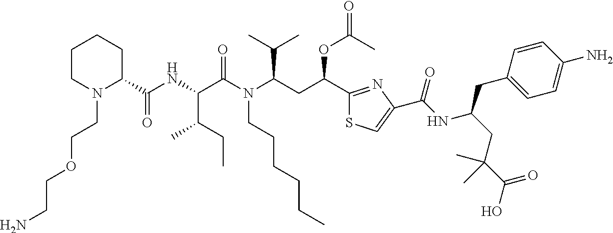 [SMILES: CCCCCCN(C(=O)[C@@H](NC(=O)[C@H]1CCCCN1CCOCCN)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 3.81 | C₄₇H₇₇- N₇O₈S | 900.23 | 451.0 (M/2 + H) |
| PA12 | 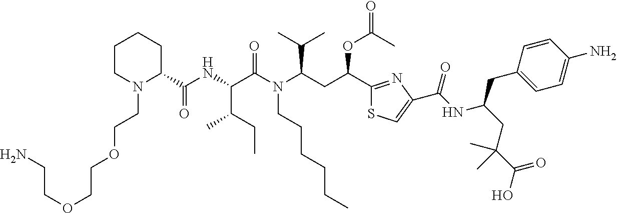 [SMILES: CCCCCCN(C(=O)[C@@H](NC(=O)[C@H]1CCCCN1CCOCCOCCN)[C@@H](C)CC)[C@H](C[C@@H](OC(C)=O)c1nc(C(=O)N[C@@H](Cc2ccc(N)cc2)CC(C)(C)C(=O)O)cs1)C(C)C] | 3.77 | C₄₉H₈₁- N₇O₉S | 944.29 | 472.8 (M/2 + H) |
| PA13 | 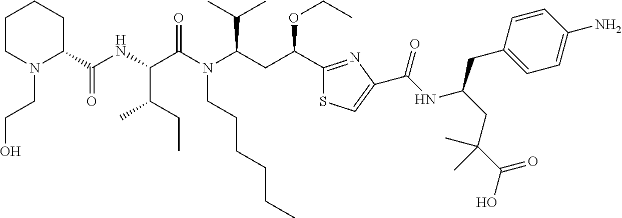 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(N)cc2)CC(C)(C)C(=O)O)cs1)C(C)C] | 4.02 | C₄₅H₇₄- N₆O₇S | 843.18 | 843.3 (M + H) |
| PA28 | 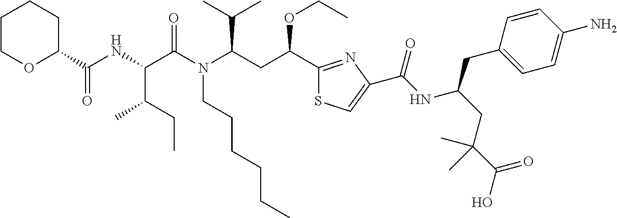 [SMILES: CCCCCCN(C(=O)[C@@H](NC(=O)[C@H]1CCCCO1)[C@@H](C)CC)[C@H](C[C@@H](OCC)c1nc(C(=O)N[C@@H](Cc2ccc(N)cc2)CC(C)(C)C(=O)O)cs1)C(C)C] | 6.76 | C₄₃H₆₉- N₅O₇S | 800.11 | 799.8 (M + H) |
| P15 | 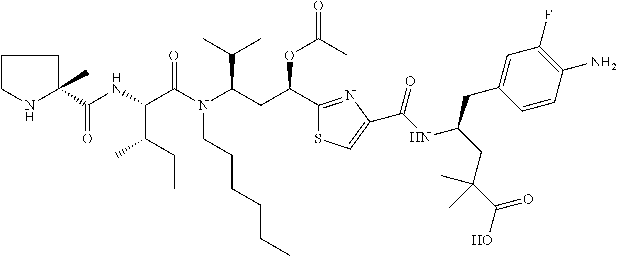 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | 4.25 | C₄₃H₆₇F- N₆O₇S | 831.10 | 416.4 (M/2 + H) |
| P22 | 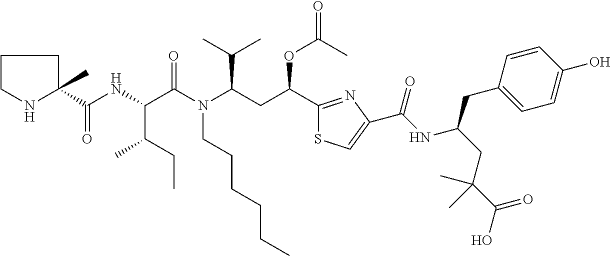 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] | 4.63 | C₄₃H₆₇- N₅O₈S | 814.10 | 814.5 (M + H) |

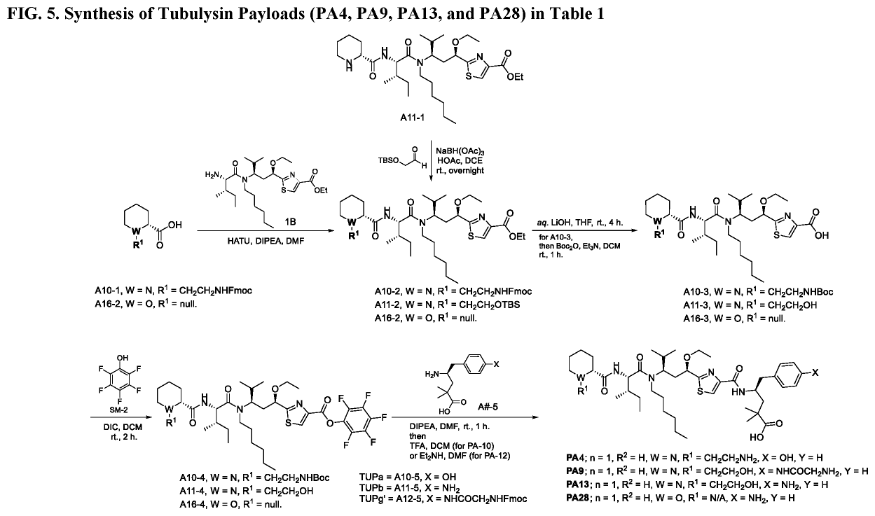

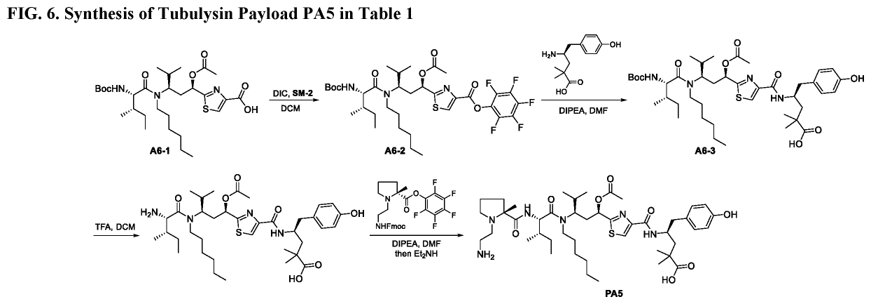

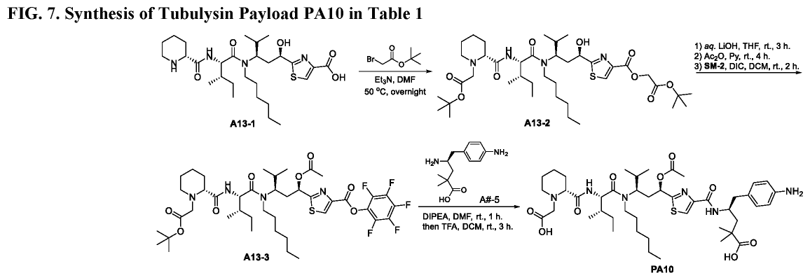

Synthesis of payload PA4 was consistent with the reaction scheme in FIG. 5.

### Synthesis of (S)-4-(2-((1R,3R)-3-((2S,3S)-2-((R)-1-(2-aminoethyl)piperidine-2-carboxamido)-N-hexyl-3-methylpentanamido)-1-ethoxy-4-methylpentyl)thiazole-4-carboxamido)-5-(4-hydroxyphenyl)-2,2-dimethylpentanoic acid (PA4)

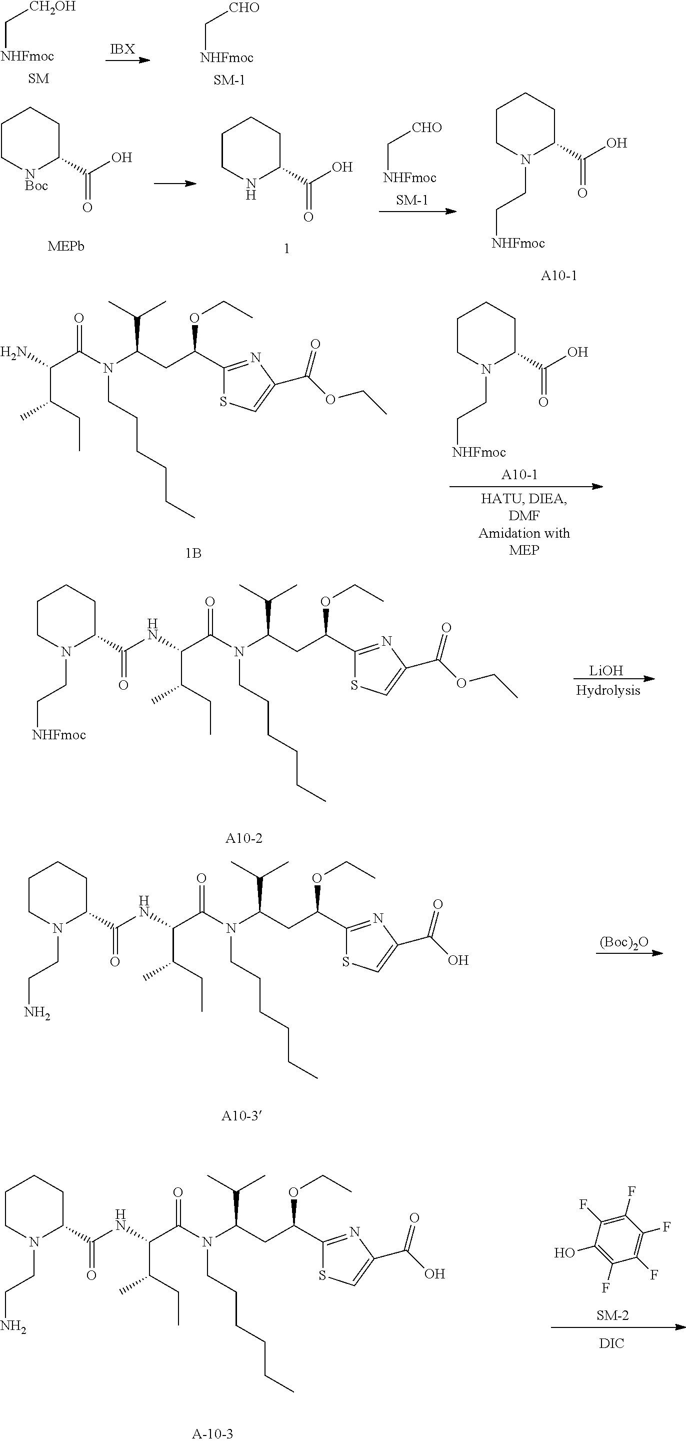

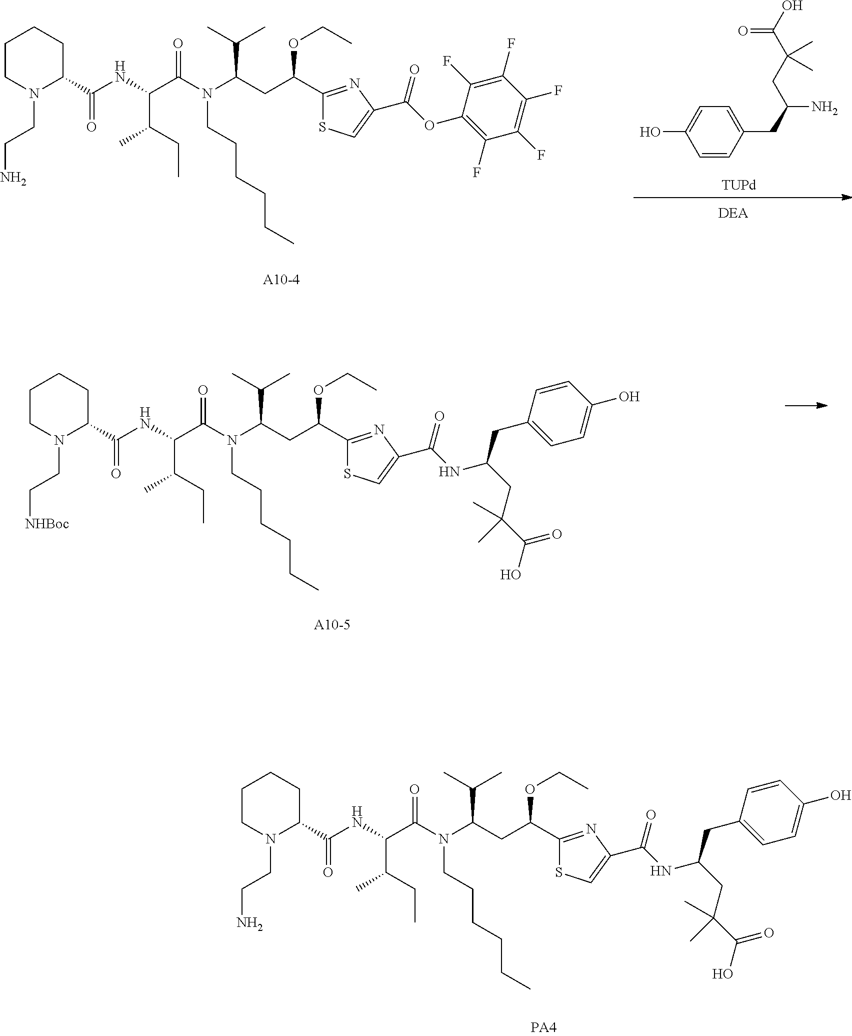

### Synthesis of (9H-fluoren-9-yl)methyl (2-oxoethyl)carbamate (SM-1)

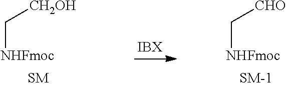

A solution of SM (283 mg, 1 mmol) and IBX (560 mg, 2 mmol) in EtOAc (20 mL) was stirred at 70° C. for 4 h. The mixture was filtered and the filtrate was concentrated to give (9H-fluoren-9-yl)methyl (2-oxoethyl)carbamate (SM-1) as a yellow oil (280 mg, 20% yield). LCMS [M+1]⁺=282.4.

### General Procedure I

### Boc Deprotection to Give (R)-piperidine-2-carboxylic acid 2

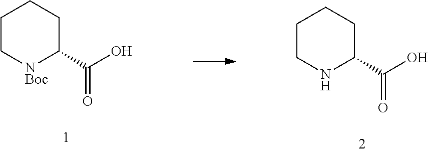 [SMILES: CC(C)(C)OC(=O)N1CCCC[C@@H]1C(=O)O ; O=C(O)[C@H]1CCCCN1]

A solution of (R)-1-(tert-butoxycarbonyl)piperidine-2-carboxylic acid (1) (229 mg, 1 mmol) in DCM (4 mL) and TFA (1 mL) was stirred at rt for 1 h. The solution was concentrated to give a (R)-piperidine-2-carboxylic acid (2) as a yellow oil (220 mg, 90% yield). LCMS [M+1]⁺=130.7.

### General Procedure II

### Amidation With MEP: Synthesis (R)-1-(2-((((9H-fluoren-9-yl)methoxy)carbonyl)amino)ethyl)piperidine-2-carboxylic acid A10-1

### Synthesis of (R)-1-(2-((((9H-fluoren-9-yl)methoxy)carbonyl)amino)ethyl)piperidine-2-carboxylic acid (A10-1)

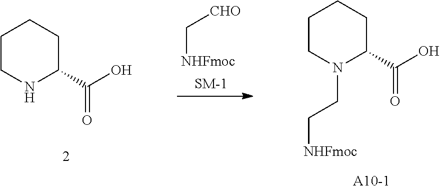

Na(OAc)₃BH₄ (246 mg, 1.16 mmol) was gently added to a stirred suspension of (R) -piperidine-2-carboxylic acid (2) (100 mg, 0.77 mmol) in CH₂Cl₂ (10 mL) at rt. Two drops of TFA was then added to quench the reaction and the quenched reaction was allowed to stir 1 h at rt. After 1 h, the solution was washed with 1 N HCl (10 mL). The layers were separated and the organic layer was washed with H₂O (10 mL). The layers were separated and further washed with 10% aq. Na₂CO₃ solution (10 mL). The layers were separated and the organic layer was washed with sat. NaHCO₃ (10 mL). The layers were separated and the organic layer was dried over Na₂SO₄ and filtered. The filtrate was concentrated and purified by prep-HPLC to afford (R)-1-(2-((((9H-fluoren-9-yl)methoxy)carbonyl)amino)ethyl)piperidine-2-carboxylic acid (3) (40 mg, 10% yield) as colorless oil. LCMS [M+1]⁺=395.3.

### General Procedure III

### Amidation With MEP: Synthesis of Ethyl 2-((1R,3R)-3-((2S,3S)-2-((R)-1-(2-((((9H-fluoren-9-yl)methoxy)carbonyl)amino)ethyl)piperidine-2-carboxamido)-N-hexyl-3-methylpentanamido)-1-ethoxy-4-methylpentyl)thiazole-4-carboxylate A10-2

### Synthesis of ethyl 2-((1R,3R)-3-((2S,3S)-2-((R)-1-(2-((((9H-fluoren-9-yl)methoxy)carbonyl)amino)ethyl)piperidine-2-carboxamido)-N-hexyl-3-methylpentanamido)-1-ethoxy-4-methylpentyl)thiazole-4-carboxylate (A10-2)

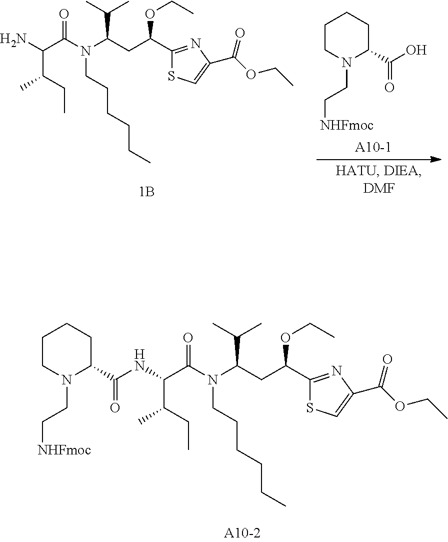

A mixture of ethyl 2-((1R,3R)-3-((2S,3S)-2-amino-N-hexyl-3-methylpentanamido)-1-ethoxy-4-methylpentyl)thiazole-4-carboxylate (1B) (50 mg, 0.1 mmol), (R)-1-(2-((((9H-fluoren-9-yl)methoxy)carbonyl)amino)ethyl)piperidine-2-carboxylic acid (A10-1) (47 mg, 0.12 mmol), HATU (57 mg, 0.15 mmol), and DIPEA (26 mg, 0.2 mmol) in DMF (2 mL) was stirred at rt for 2 h. Water (10 mL) was added to quench and the reaction was extracted with EtOAc (3×20 mL). The combined organic layers were concentrated to give ethyl 2-((1R,3R)-3-((2S,3S)-2-((R)-1-(2-((((9H-fluoren-9-yl)methoxy)carbonyl)amino)ethyl)piperidine-2-carboxamido)-N-hexyl-3-methylpentanamido)-1-ethoxy-4-methylpentyl)thiazole-4-carboxylate (A10-2) as a yellow oil (70 mg, 80% yield). A10-2 was used directly in the next step without purification. LCMS [M+1]⁺=874.5.

### General Procedure IV

### Hydrolysis: Synthesis of A10-3′

### Synthesis of 2-((1R,3R)-3-((2S,3S)-2-((R)-1-(2-aminoethyl)piperidine-2-carboxamido)-N-hexyl-3-methylpentanamido)-1-ethoxy-4-methylpentyl)thiazole-4-carboxylic acid (A10-3′)

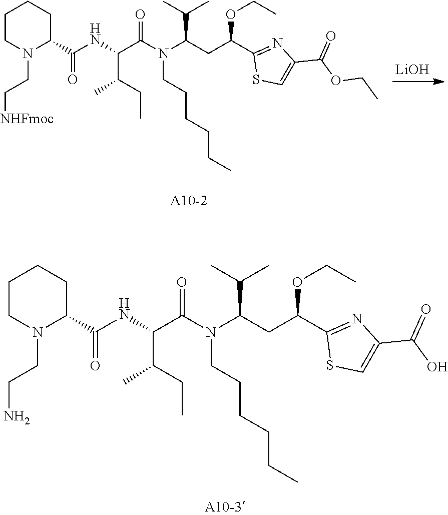

A mixture of ethyl 2-((1R,3R)-3-((2S,3S)-2-((R)-1-(2-((((9H-fluoren-9-yl)methoxy)carbonyl)amino)ethyl)piperidine-2-carboxamido)-N-hexyl-3-methylpentanamido)-1-ethoxy-4-methylpentyl)thiazole-4-carboxylate (A10-32) (70 mg, 0.08 mmol) and LiOH (32 mg, mmol) in THF (2 mL) and H₂O (1 mL) was stirred at rt for 2 h. Use of HCl adjusted the pH to <7. H₂O (10 mL) was added and the mixture was extracted with EtOAc (3×20 mL). The combined organic layers were concentrated and purified by prep-HPLC to afford 2-((1R,3R)-3-((2S,3S)-2-((R)-1-(2-aminoethyl)piperidine-2-carboxamido)-N-hexyl-3-methylpentanamido)-1-ethoxy-4-methylpentyl)thiazole-4-carboxylic acid (A10-3′) (15 mg, 30% yield) as a white solid. LCMS [M+1]⁺=624.3.

### General Procedure V

### N-Boc Protection: Synthesis of A10-3

### Synthesis of 2-((1R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylic acid (A10-3)

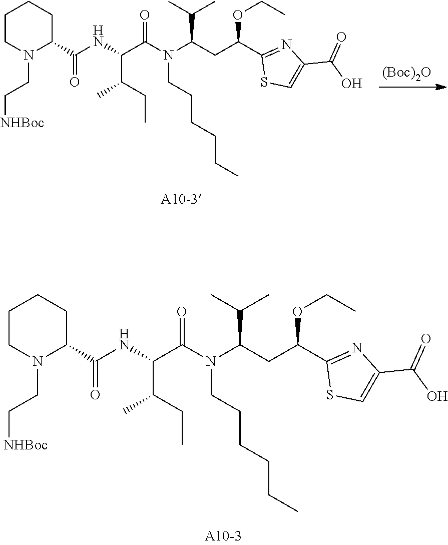 [SMILES: "(Boc)2O" ; CCCCCCN(C(=O)[C@@H](NC(=O)[C@H]1CCCCN1CCNC(=O)OC(C)(C)C)[C@@H](C)CC)[C@H](C[C@@H](OCC)c1nc(C(=O)O)cs1)C(C)C ; CCCCCCN(C(=O)[C@@H](NC(=O)[C@H]1CCCCN1CCNC(=O)OC(C)(C)C)[C@@H](C)CC)[C@H](C[C@@H](OCC)c1nc(C(=O)O)cs1)C(C)C]

A mixture of 2-((1R,3R)-342S,3S)-2-((R)-1-(2-aminoethyl)piperidine-2-carboxamido)-N-hexyl-3-methylpentanamido)-1-ethoxy-4-methylpentyl)thiazole-4-carboxylic acid (A10-3′) (15 mg, 0.024 mmol), (Boc)₂O (10 mg, 0.048 mmol), and TEA (5 mg, 0.048 mmol) in CH₂Cl₂ (5 mL) was stirred at rt for 2 h. The reaction mixture was concentrated and purified by prep-HPLC to afford 2-((1R,3R)-342S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylic acid (A10-3) (20 mg, 90% yield) as a white solid. LCMS [M+1]⁺=724.3.

### General Procedure VI

### Carboxylic Acid Group Activation: Synthesis of A10-4

### Synthesis of perfluorophenyl 241R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylate (A10-4)

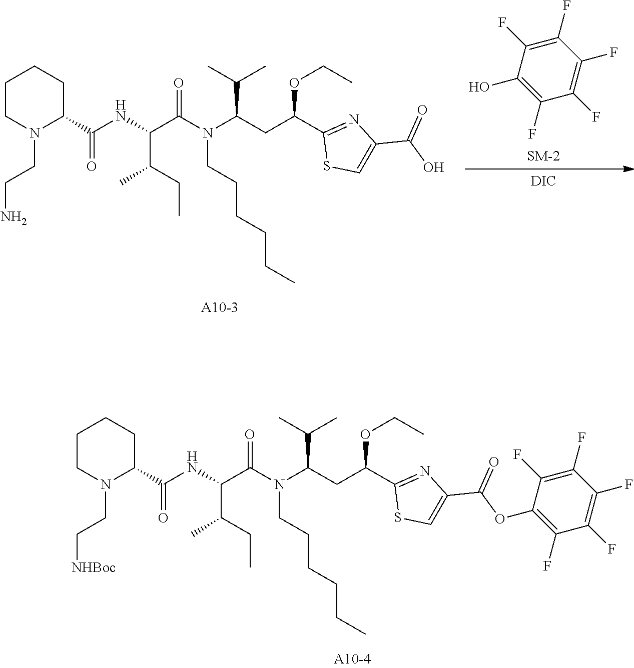

A solution of 2-((1R,3R)-34(2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylic acid (A10-3) (20 mg, 0.02 mmol) and 2,3,4,5,6-pentafluorophenol (SM-2) (6 mg, 0.03 mmol) in CH₂Cl₂ (4 mL) was added to diisopropylcarbodiimide (4 mg, 0.03 mmol). The mixture was stirred at rt for 1 h. The mixture was then concentrated to give perfluorophenyl 2-((1R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylate (A10-4) as a yellow oil (25 mg, 80% yield). A10-4 was used directly in the next step without purification. LCMS [M+1]⁺=890.3.

### General Procedure VII

### Amide Formation: Synthesis of A10-5

### Synthesis of (S)-4-(241R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxamido)-5-(4-hydroxyphenyl)-2,2-dimethylpentanoic acid (A10-5)

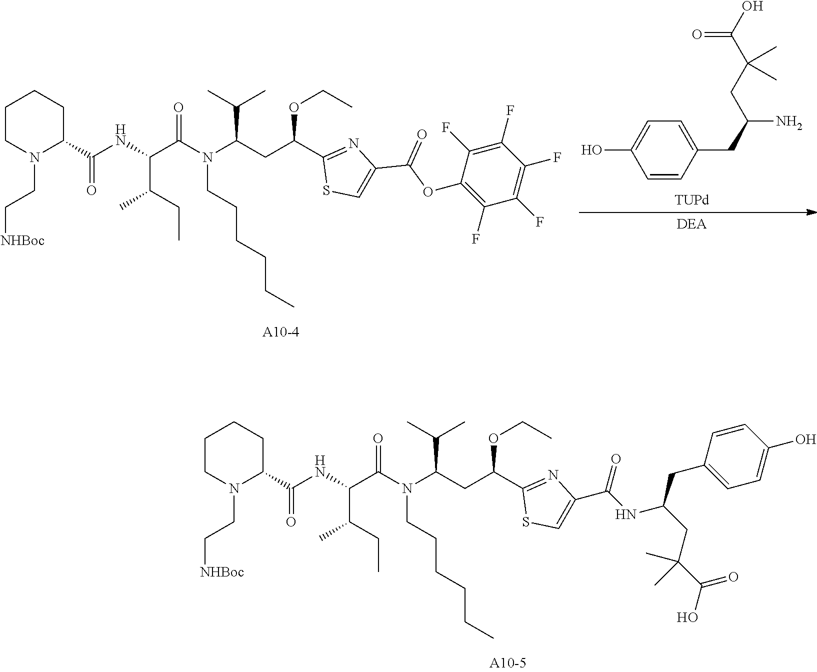

A solution of perfluorophenyl 2-((1R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylate (A10-4) (25 mg, 0.02 mmol) and (S)-4-amino-5-(4-hydroxyphenyl)-2,2-dimethylpentanoic acid (TUPd, 7 mg, 0.03 mmol) in CH₂Cl₂ (4 mL) was added to DIPEA (4 mg, 0.03 mmol). The mixture was stirred at rt for 1 h. The mixture was then concentrated to give (S)-4-(2-((1R,3R)-3-((2S,3S)-2-((R)-1-(2-((tert-butoxy carbonyl)amino)ethyl)piperidine-2-carboxamido)-N-hexyl-3-methylpentanamido)-1-ethoxy-4-methylpentyl)thiazole-4-carboxamido)-5-(4-hydroxyphenyl)-2,2-dimethylpentanoic acid (A10-5) as a yellow oil (30 mg, 80% yield). A10-5 was used directly in the next step without purification. LCMS [M+1]⁺=943.7.

### Synthesis of (S)-4-(2-((1R,3R)-342S,3S)-2-((R)-1-(2-aminoethyl)piperidine-2-carboxamido)-N-hexyl-3-methylpentanamido)-1-ethoxy-4-methylpentyl)thiazole-4-carboxamido)-5-(4-hydroxyphenyl)-2,2-dimethylpentanoic acid (PA4)

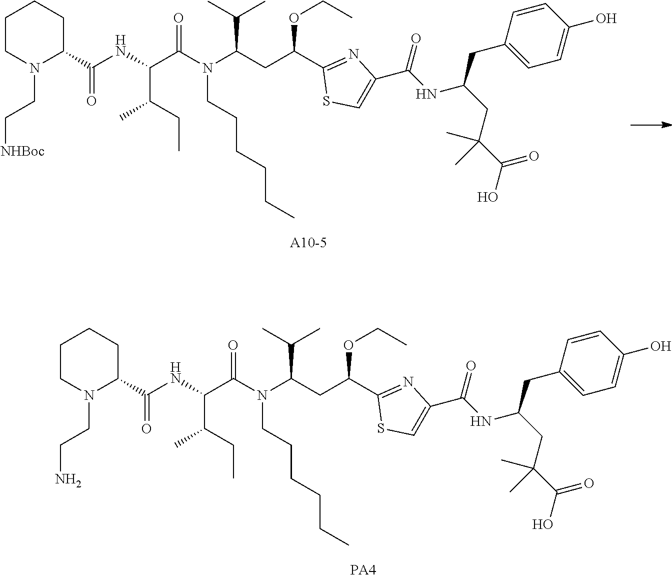 [SMILES: CCCCCCN(C(=O)[C@@H](NC(=O)[C@H]1CCCCN1CCNC(=O)OC(C)(C)C)[C@@H](C)CC)[C@H](C[C@@H](OCC)c1nc(C(=O)N[C@@H](Cc2ccc(O)cc2)CC(C)(C)C(=O)O)cs1)C(C)C ; CCCCCCN(C(=O)[C@@H](NC(=O)[C@H]1CCCCN1CCN)[C@@H](C)CC)[C@H](C[C@@H](OCC)c1nc(C(=O)N[C@@H](Cc2ccc(O)cc2)CC(C)(C)C(=O)O)cs1)C(C)C]

A solution of (S)-4-(2-((1R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxamido)-5-(4-hydroxyphenyl)-2,2-dimethylpentanoic acid (A10-5) (30 mg, 0.016 mmol) in CH₂Cl₂ (4 mL) and TFA (1 mL) was stirred at rt for 1 h. The mixture was then concentrated and the residue was purified by prep-HPLC to give (S)-4-(2-((1R,3R)-3-((2S,3S)-2-((R)-1-(2-aminoethyl)piperidine-2-carboxamido)-N-hexyl-3-methylpentanamido)-1-ethoxy-4-methylpentyl)thiazole-4-carboxamido)-5-(4-hydroxyphenyl)-2,2-dimethylpentanoic acid (PA4) as a white solid (8.8 mg, 67% yield). ¹H NMR (500 MHz, DMSO-d₆) δ 9.19 (br s, 1H), 8.45 (br s, 1H), 8.17-8.15 (m, 2H), 6.92 (d, J=8.2 Hz, 2H), 6.63 (d, J=8.2 Hz, 2H), 4.46 (s, 1H), 4.26 (d, J=10.7 Hz, 1H), 4.12 (s, 1H), 4.00 (s, 1H), 3.75-3.50 (m, 3H), 3.15-2.66 (m, 8H), 2.38-2.12 (m, 4H), 1.99-1.79 (m, 5H), 1.72-1.42 (m, 8H), 1.31-1.11 (m, 13H), 0.98-0.79 (m, 20H). LCMS [M+1]⁺=843.3.

Synthesis of payloads PA9, PA13, and PA28 was consistent with FIG. 5.

Payloads, PA9, PA13, and PA28 were prepared following similar procedures described for compound PA4 as consistent with the scheme below.

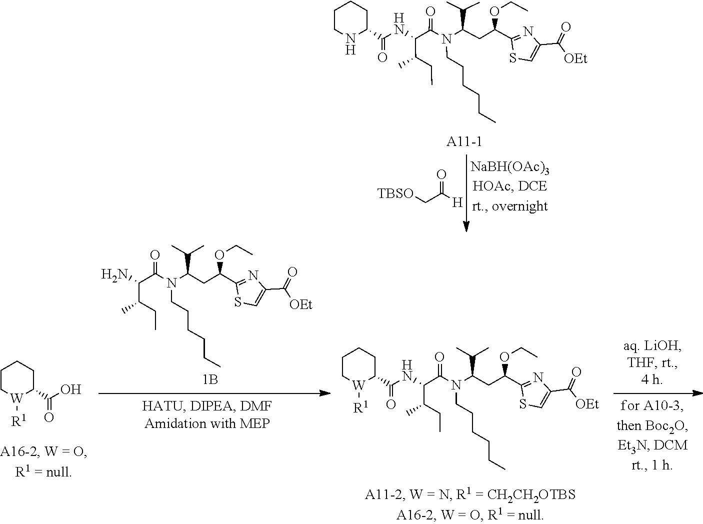

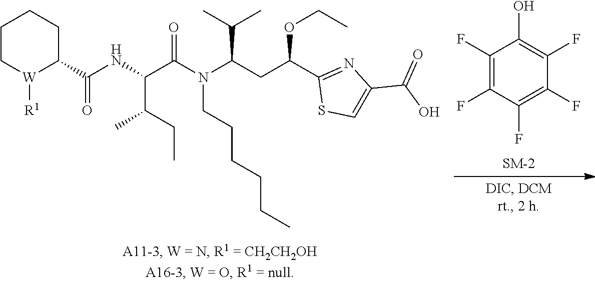

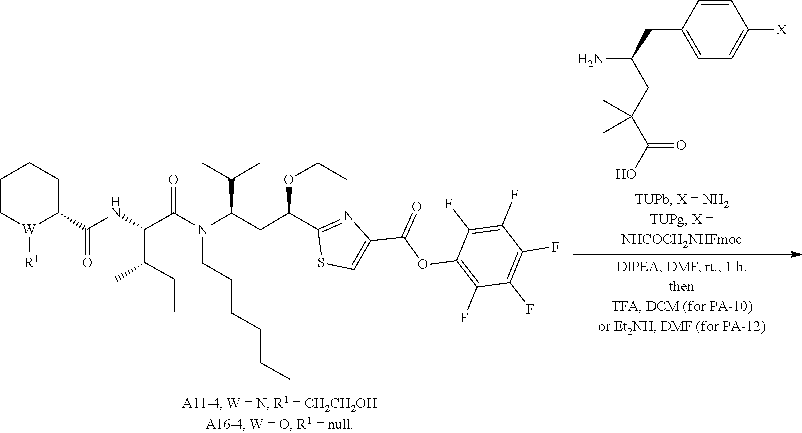

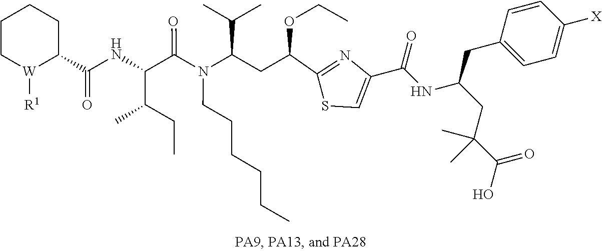

| Compound No. | n/R² | W | R¹ | X | Y |
|---|---|---|---|---|---|
| PA9 | 1/H | N | CH₂CH₂OH | NHCOCH₂NH₂ | H |
| PA13 | 1/H | N | CH₂CH₂OH | NH₂ | H |
| PA28 | 1/H | O | N/A | NH₂ | H |

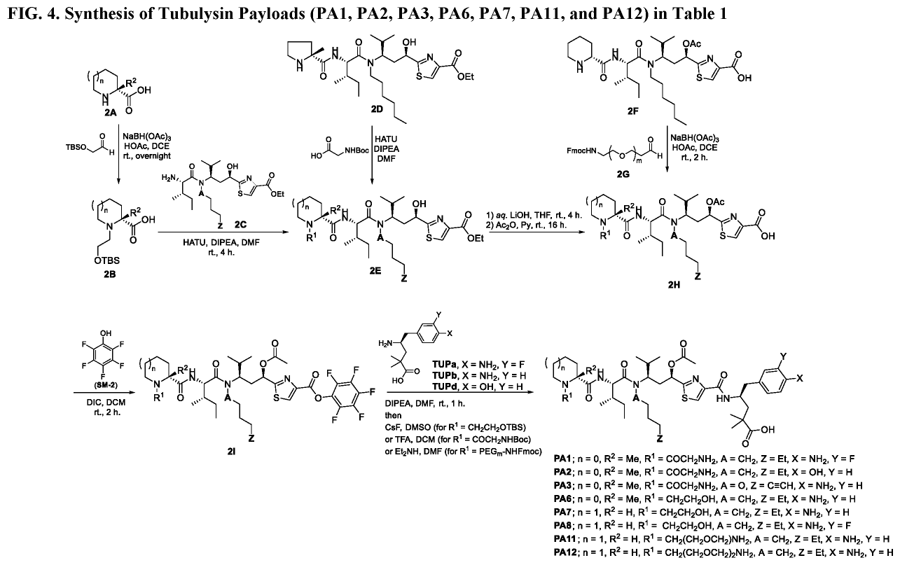

Synthesis of payloads PA1-PA3, PA6-PA8, PA11, and PA12 was consistent with FIG. 4.

Payloads, PA1-PA3, PA6-PA8, PA11, and PA12 were prepared following similar procedures described for compound PA4 consistent with the scheme below from intermediate 2H.

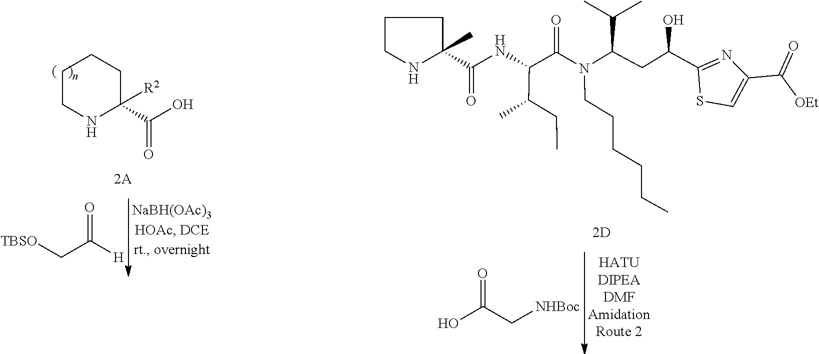

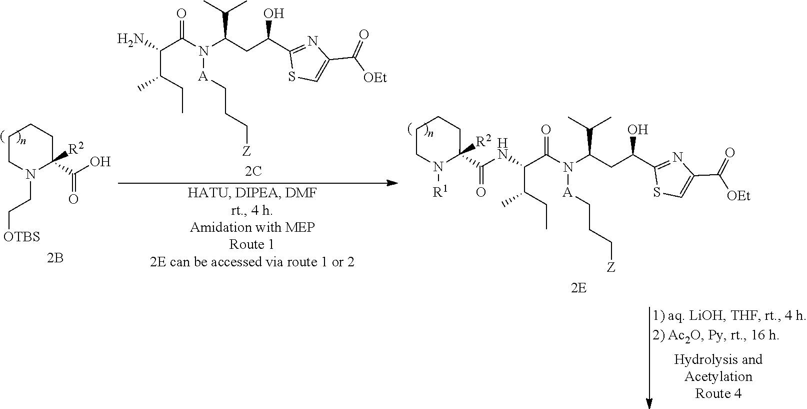

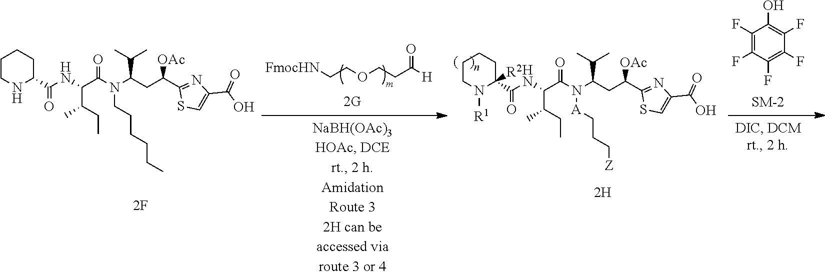

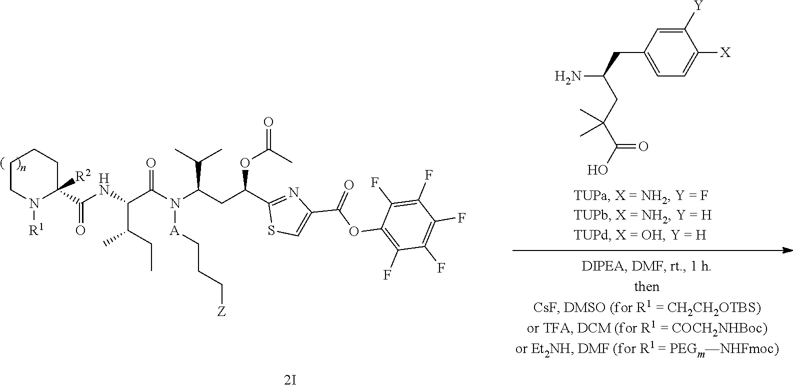

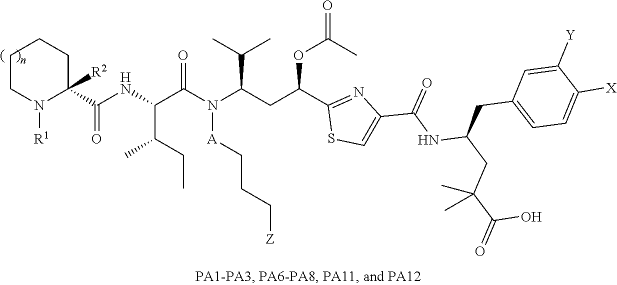

| Compound No. | n/R² | R¹ | A/Z | X | Y |
|---|---|---|---|---|---|
| PA1 | 0/Me | COCH₂NH₂ | CH₂/Et | NH₂ | F |
| PA2 | 0/Me | COCH₂NH₂ | CH₂/Et | OH | H |
| PA3 | 0/Me | CH₂CH₂OH | O/C≡CH | NH₂ | H |
| PA6 | 0/Me | CH₂CH₂OH | CH₂/Et | NH₂ | H |
| PA7 | 1/H | CH₂CH₂OH | CH₂/Et | NH₂ | H |
| PA8 | 1/H | CH₂CH₂OH | CH₂/Et | NH₂ | F |
| PA11 | 1/H | CH₂(CH₂OCH₂)NH₂ | CH₂/Et | NH₂ | H |
| PA12 | 1/H | CH₂(CH₂OCH₂)₂NH₂ | CH₂/Et | NH₂ | H |

Compounds of intermediate 2E were prepared via Routes 1 or 2. Route 1 utilizes General Procedure II, whilst Route 2 uses General Procedure III. Compounds of intermediate 2E was converted to compounds of intermediate 2H via Route 4 using General Procedure IV. Compounds of intermediate 2H was also accessed using Route 3 using General Procedure III.

### General Procedure VIII

### Acetylation of the Hydrolysis Product 2E′ to Give 2H

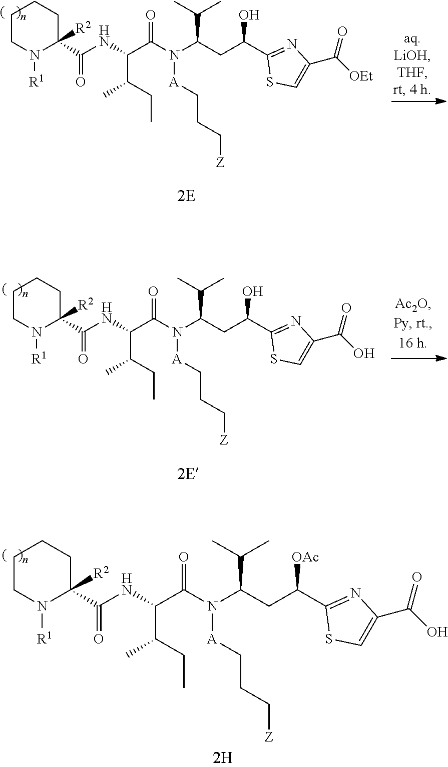

Compound 2H was prepared from compound 2E consistent with the scheme above.

TABLE 2
Tubulysin Payloads Modified on Tup

| Payload | | | | | |
|---|---|---|---|---|---|
| No. | Structures | cLogP | MF | MW | ESI m/z |
| PA14 | 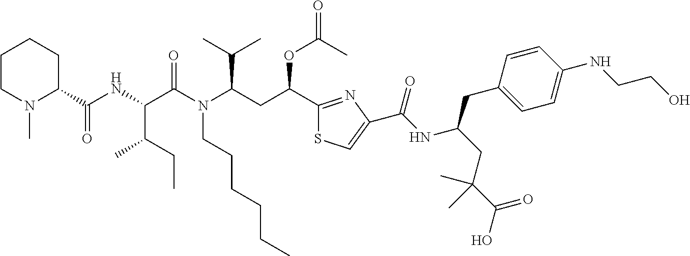 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCO)cc2)CC(C)(C)C(=O)O)cs1)C(C)C] | 3.94 | C₄₆H₇₄N₆O₈S | 871.19 | 871.5 (M + H) |
| PA15 | 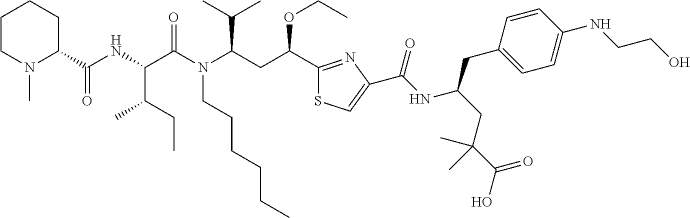 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCO)cc2)CC(C)(C)C(=O)O)cs1)C(C)C] | 4.50 | C₄₆H₇₆N₆O₇S | 857.21 | 857.5 (M + H) |
| PA16 | 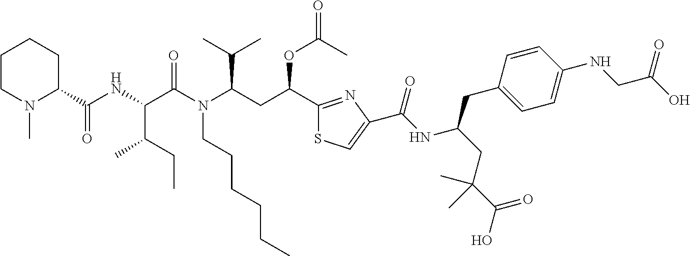 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C] | 2.66 | C₄₆H₇₂N₆O₉S | 885.18 | 885.5 (M + H) |
| PA17 | 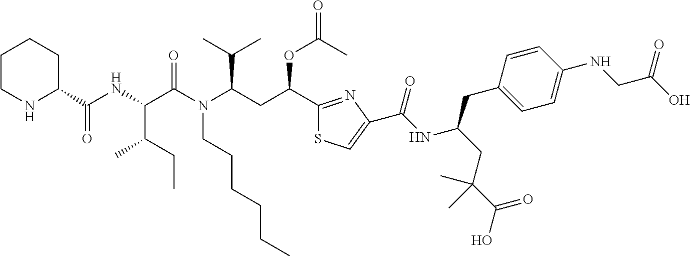 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C] | 2.48 | C₄₅H₇₀N₆O₉S | 871.15 | 871.2 (M + H) |
| PA18 | 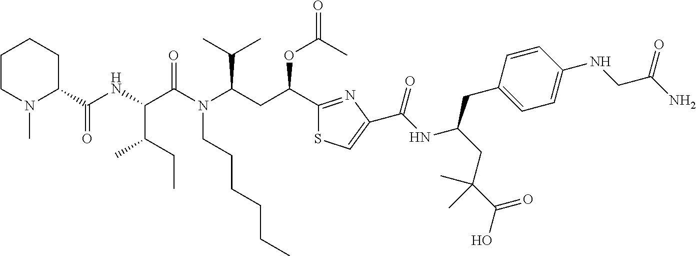 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(N)=O)cc2)CC(C)(C)C(=O)O)cs1)C(C)C] | 3.31 | C₄₆H₇₃N₇O₈S | 884.19 | 443.0 (M/2 + H) |
| PA19 | 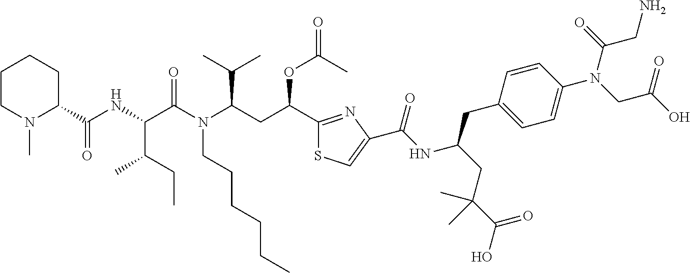 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N(CC(=O)O)C(=O)CN)cc2)CC(C)(C)C(=O)O)cs1)C(C)C] | 2.70 | C₄₈H₇₇N₇O₉S | 928.24 | 464.7 (M/2 + H) |
| PA20 | 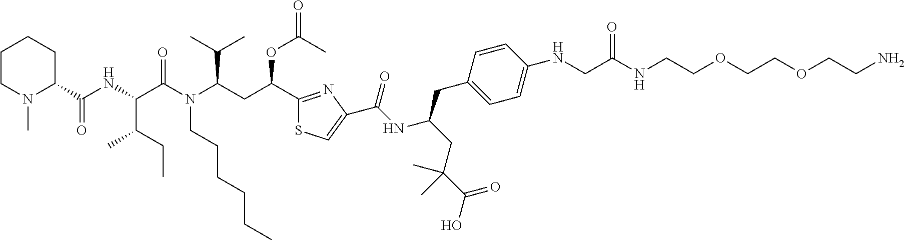 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCN)cc2)CC(C)(C)C(=O)O)cs1)C(C)C] | 2.95 | C₅₂H₈₆N₈O₁₀S | 1015.37 | 508.3 (M/2 + H) |
| PA29 | 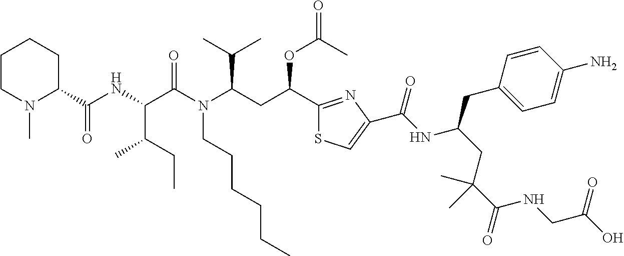 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)NCC(=O)O)cs1)C(C)C] | 3.15 | C₄₆H₇₃N₇O₈S | 884.19 | 442.9 (M/2 + H) |
| PA30 | 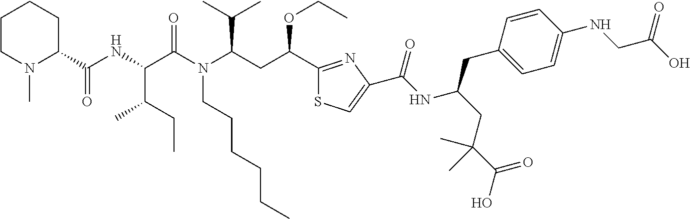 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCC(=O)O)cc2)CC(C)(C)C(=O)O)cs1)C(C)C] | 3.22 | C₄₆H₇₄N₆O₈S | 871.19 | 871.4 (M + H) |

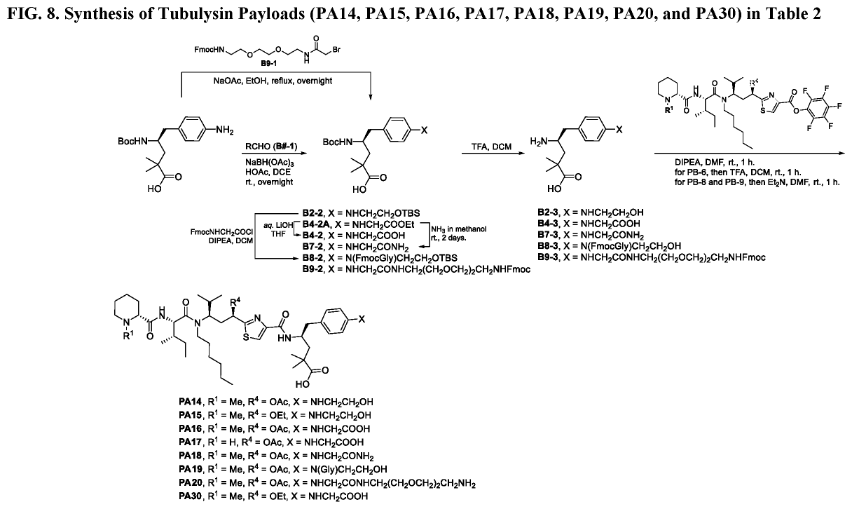

Synthesis of payloads PA14-PA20 and PA30 is consistent with FIG. 8.

Payloads PA14-PA20 and PA30 were prepared accordingly to General Procedure VII and were consistent with the Scheme below.

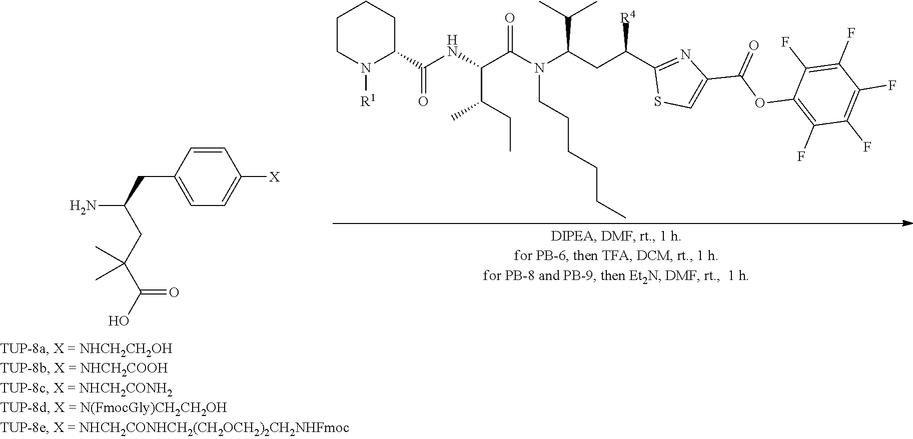

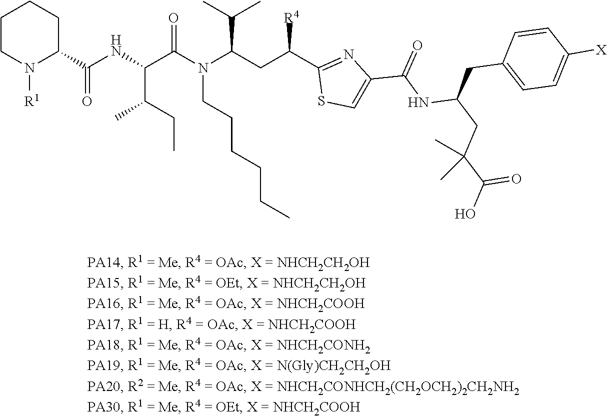

| Payload No. | R¹ | R⁴ | X |
|---|---|---|---|
| PA14 | Me | OAc | NHCH₂CH₂OH |
| PA15 | Me | OEt | NHCH₂CH₂OH |
| PA116 | Me | OAc | NHCH₂COOH |
| PA17 | H | OAc | NHCH₂COOH |
| PA18 | Me | OAc | NHCH₂CONH₂ |
| PA19 | Me | OAc | N(Gly)CH₂CH₂OH |
| PA20 | Me | OAc | NHCONHCH₂(CH₂OCH₂)₂CH₂NH₂ |
| PA30 | Me | OEt | NHCH₂COOH |

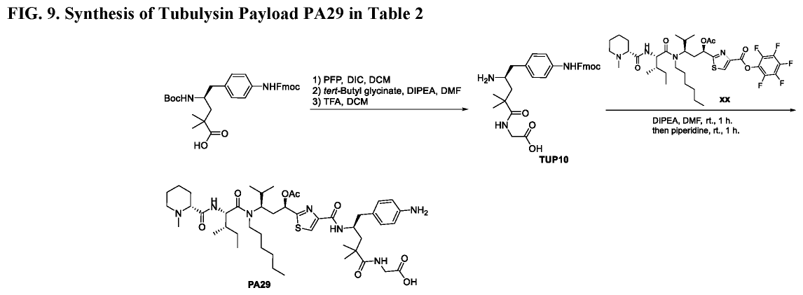

Synthesis of payload PA29 is consistent with FIG. 9.

Payload PA29 was prepared accordingly to General Procedure VII and was consistent with the scheme below.

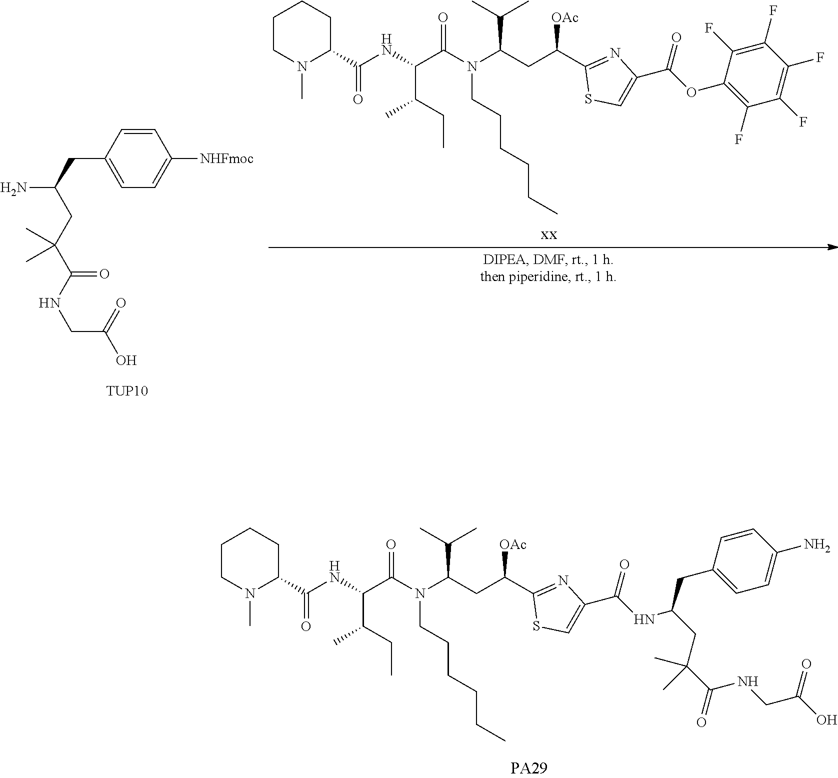

TABLE 3
Tubulysin Payloads Modified on Tuv

| Pay- | | | | | |
|---|---|---|---|---|---|
| load | | | | | ESI |
| No. | Structures | cLogP | MF | MW | m/z |
| PA21 | 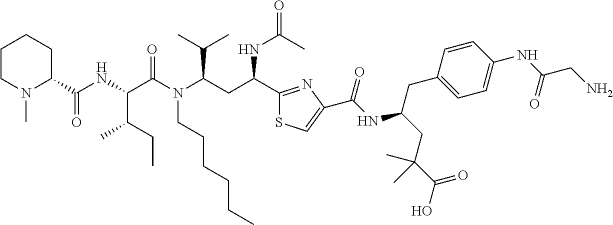 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(NC(=O)CN)cc2)CC(C)(C)C(=O)O)cs1)C(C)C] | 2.80 | C₄₆H₇₄- N₈O₇S | 883.21 | 883.3 (M + H) |
| PA22 | 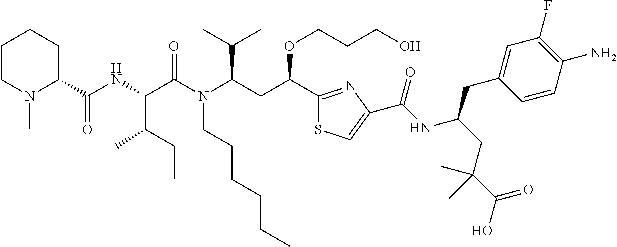 [SMILES: CCCCCCN(C(=O)[C@@H](NC(=O)[C@H]1CCCCN1C)[C@@H](C)CC)[C@H](C[C@@H](OCCCO)c1nc(C(=O)N[C@@H](Cc2ccc(N)c(F)c2)CC(C)(C)C(=O)O)cs1)C(C)C] | 4.06 | C₄₅H₇₃- FN₆O₇S | 861.17 | 861.5 (M + H) |
| PA23 | 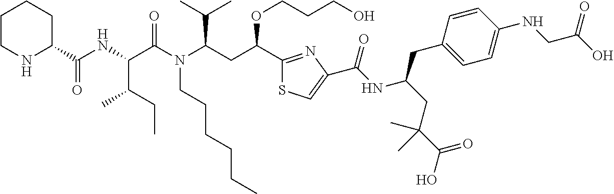 [SMILES: CCCCCCN(C(=O)[C@@H](NC(=O)[C@H]1CCCCN1)[C@@H](C)CC)[C@H](C[C@@H](OCCCO)c1nc(C(=O)N[C@@H](Cc2ccc(NCC(=O)O)cc2)CC(C)(C)C(=O)O)cs1)C(C)C] | 2.05 | C₄₆H₇₄- N₆O₉S | 887.19 | 887.4 (M + H) |
| PA24 | 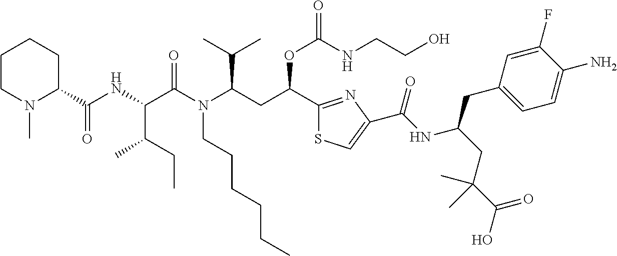 [SMILES: CCCCCCN(C(=O)[C@@H](NC(=O)[C@H]1CCCCN1C)[C@@H](C)CC)[C@H](C[C@@H](OC(=O)NCCO)c1nc(C(=O)N[C@@H](Cc2ccc(N)c(F)c2)CC(C)(C)C(=O)O)cs1)C(C)C] | 3.68 | C₄₅H₇₂- FN₇O₈S | 890.17 | 890.2 (M + H) |
| PA25 | 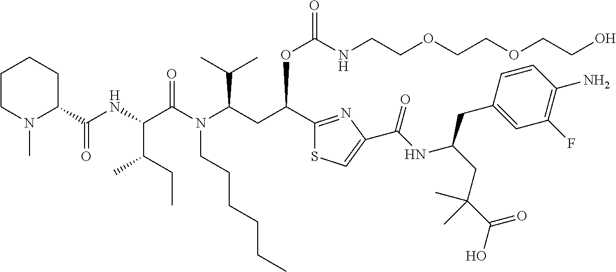 [SMILES: CCCCCCN(C(=O)[C@@H](NC(=O)[C@H]1CCCCN1C)[C@@H](C)CC)[C@H](C[C@@H](OC(=O)NCCOCCOCCO)c1nc(C(=O)N[C@@H](Cc2ccc(N)c(F)c2)CC(C)(C)C(=O)O)cs1)C(C)C] | 3.59 | C₄₉H₈₀F- N₇O₁₀S | 978.28 | 490.0 (M/2 + H) |
| PA26 | 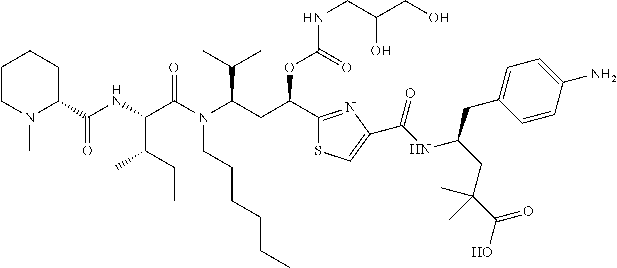 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(N)cc2)CC(C)(C)C(=O)O)cs1)C(C)C] | 2.91 | C₄₆H₇₅- N₇O₉S | 902.21 | 902.2 (M + H) |
| PA27 | 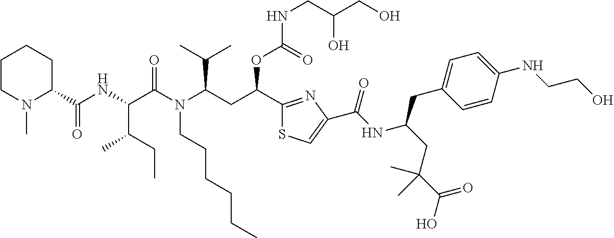 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(NCCO)cc2)CC(C)(C)C(=O)O)cs1)C(C)C] | 2.51 | C₄₈H₇₉- N₇O₁₀S | 946.26 | 946.2 (M + H) |

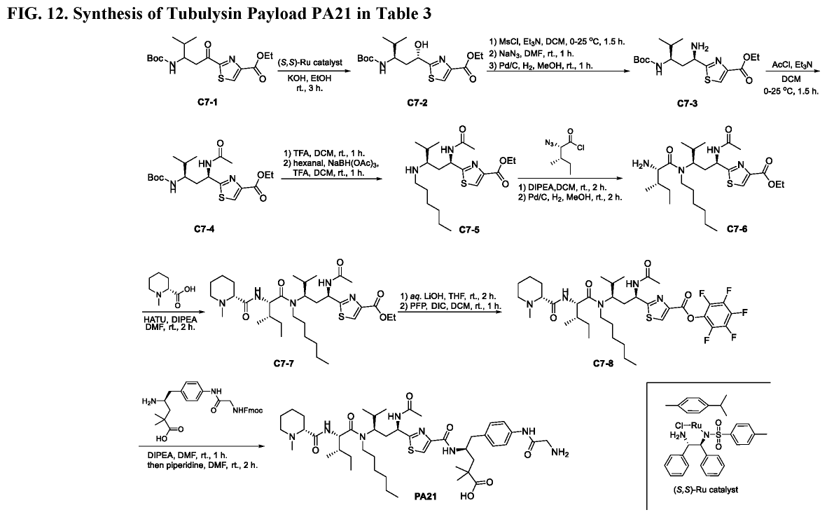

Synthesis of payload PA21 was consistent with FIG. 12.

Payload PA21 was prepared following similar procedures described for compound PA4 and was consistent with the scheme below.

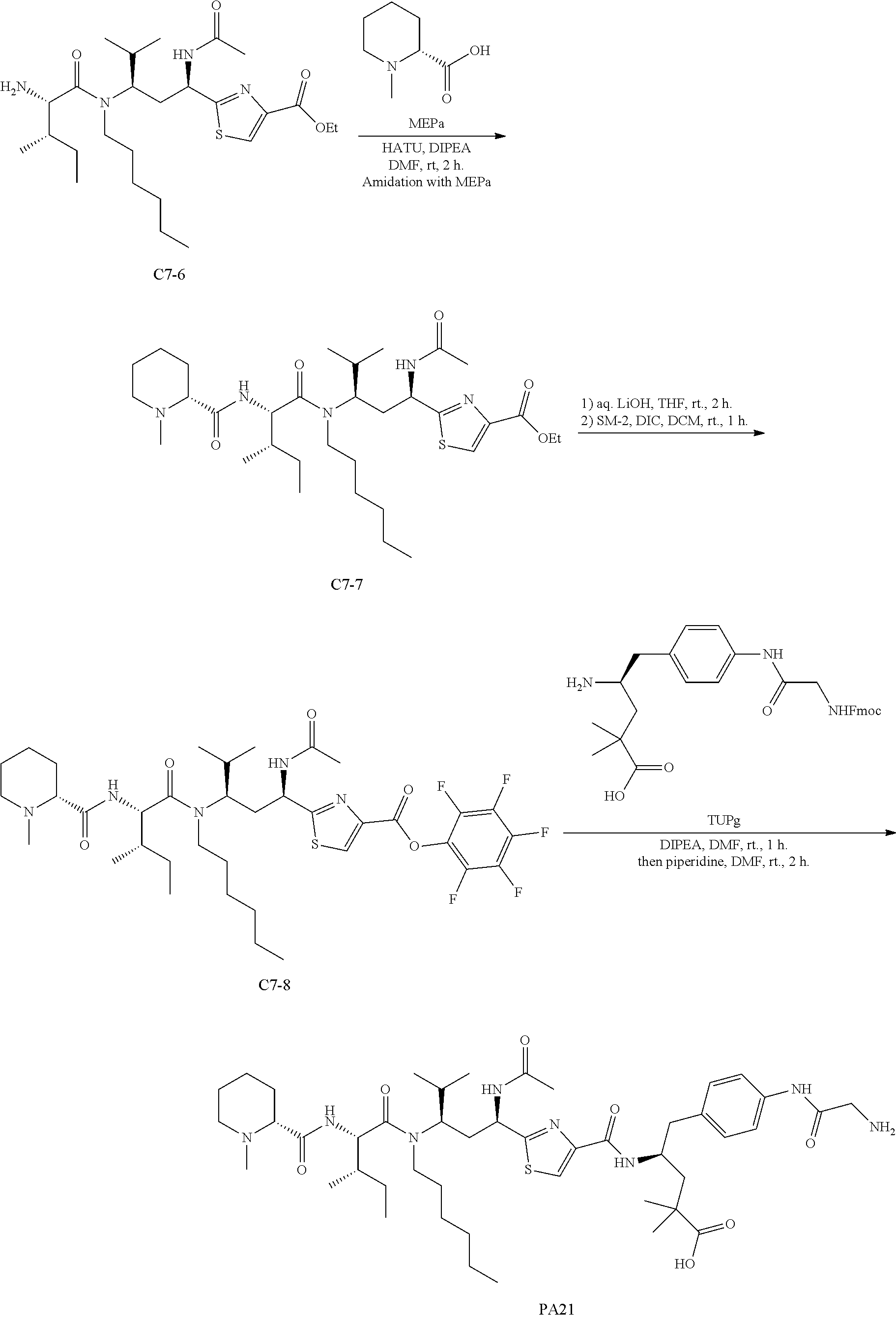

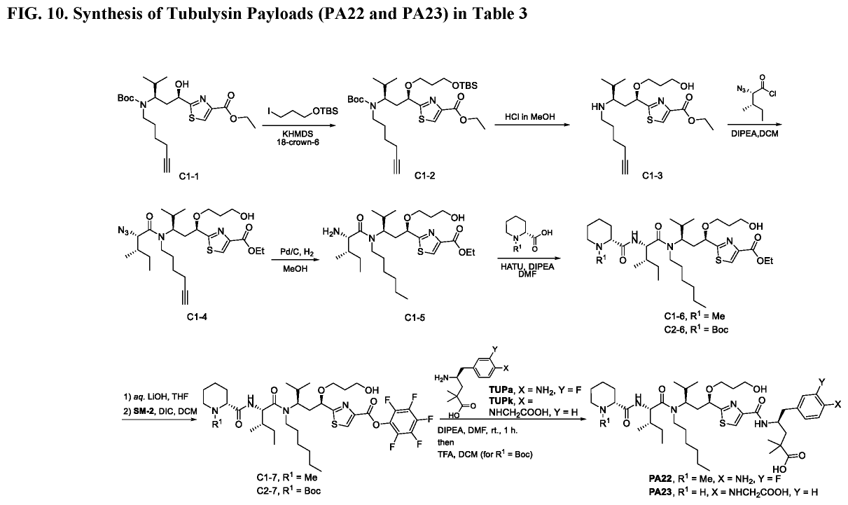

Synthesis of payloads PA22 and PA23 was consistent with FIG. 10.

Payloads PA22 and PA23 were prepared following similar procedures described for compound PA4 and were consistent with the scheme below.

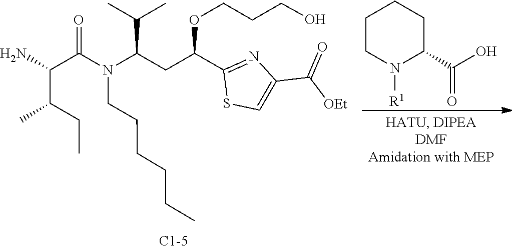

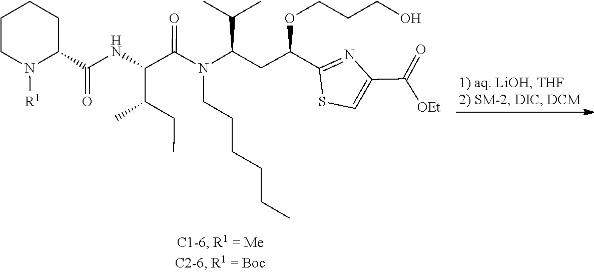

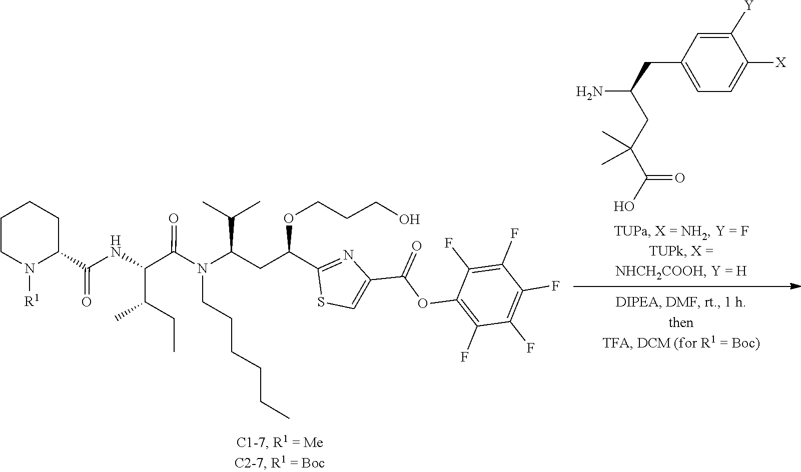

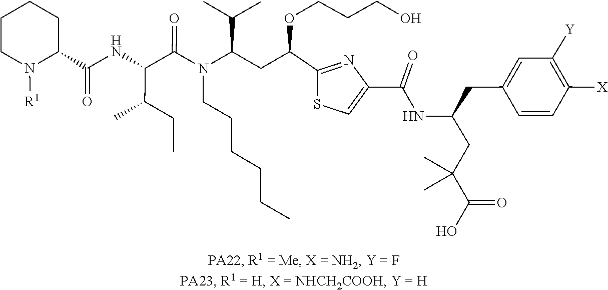

| Payload No. | R¹ | X | Y |
|---|---|---|---|
| PA22 | Me | NH₂ | F |
| PA23 | H | NHCH₂COOH | H |

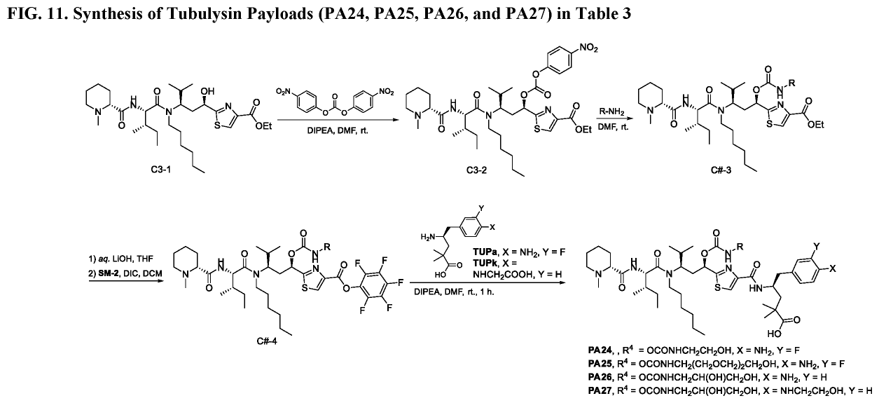

Synthesis of payloads PA24-PA27 were consistent with FIG. 11.

Payloads PA24-PA27 were prepared from intermediate C #-4 according to General Procedure VII and was consistent with the scheme below.

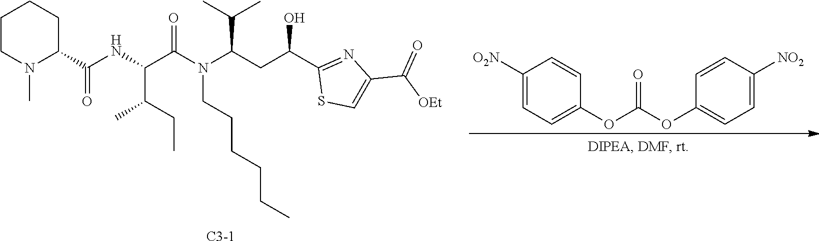 [SMILES: CCCCCCN(C(=O)[C@@H](NC(=O)[C@H]1CCCCN1C)[C@@H](C)CC)[C@H](C[C@@H](O)c1nc(C(=O)OCC)cs1)C(C)C ; O=C(Oc1ccc([N+](=O)[O-])cc1)Oc1ccc([N+](=O)[O-])cc1 ; "C3-1"]

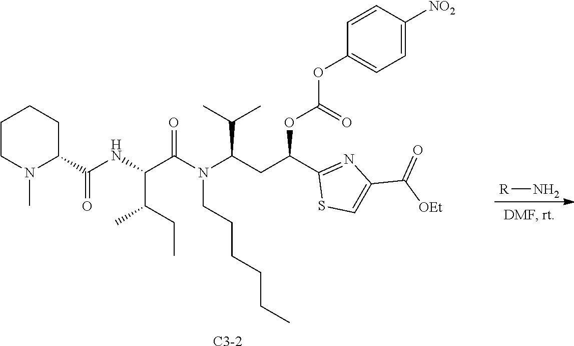

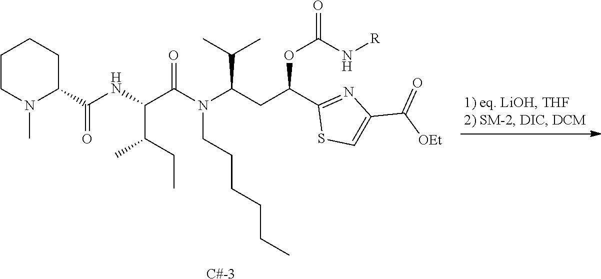

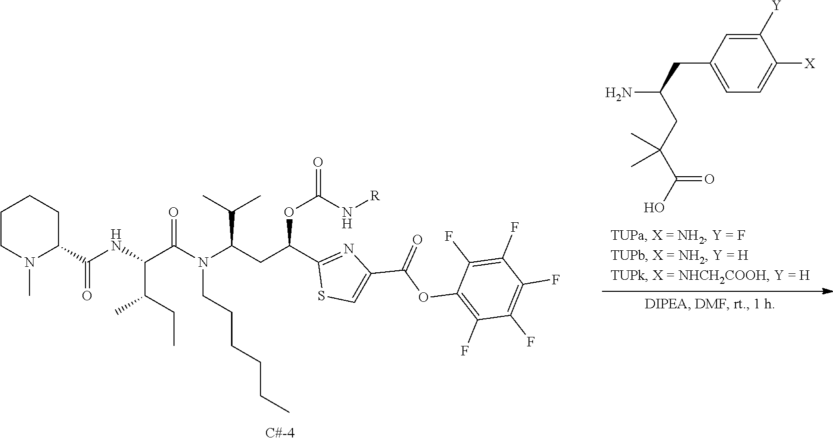

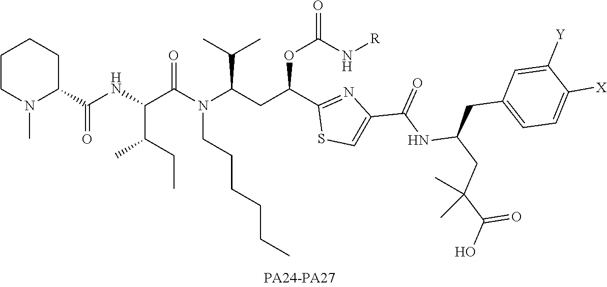

| Payload No. | R | X | Y |
|---|---|---|---|
| PA24 | OCONHCH₂CH₂OH | NH₂ | F |
| PA25 | OCONHCH₂(CH₂OCH₂)₂CH₂OH | NH₂ | F |
| PA26 | OCONHCH₂CH(OH)CH₂OH | NH₂ | H |
| PA27 | OCONHCH₂CH(OH)CH₂OH | NHCH₂CH₂OH | H |

### General Procedure IX

### Synthesis of Carbamates C #-3

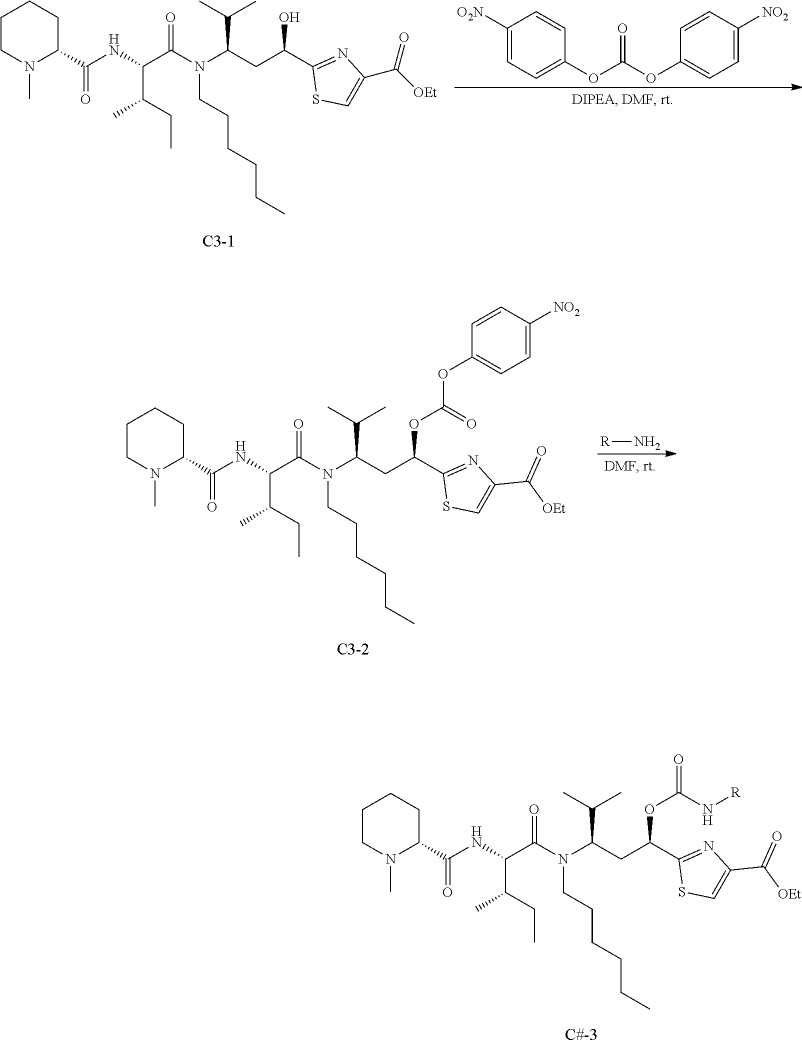

To a solution of compound C3-1 (1.0 equiv) in DMF (25 mM) was added DIPEA (3.0 equiv) and 4-nitrobenzoic anhydride (5.0 equiv). The mixture was stirred at room temperature for sixteen hours, and monitored by LCMS. The reaction solution was diluted with water and extracted with ethyl acetate (×3). The combined organic solution was dried over anhydrous sodium sulfate, filtered, and concentrated in vacuo. The residue was dissolved in DMF (50 mM). To the solution was added amine (RNH₂) (2.0 equiv) and DIPEA (2.0 equiv). The mixture was stirred at room temperature for an hour, and monitored by LCMS. The resulting mixture was purified directly by reverse phase flash chromatography (5-95% acetonitrile in water) to give compounds of C #-3 (over two steps from C3-1). Compounds of C #-3 were then treated under appropriate reaction conditions as described for compound PA4 consistent with the scheme below to obtain compounds PA24-PA27.

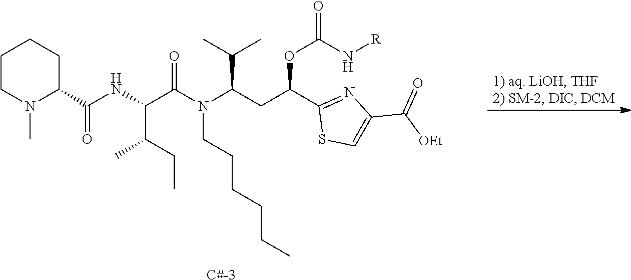

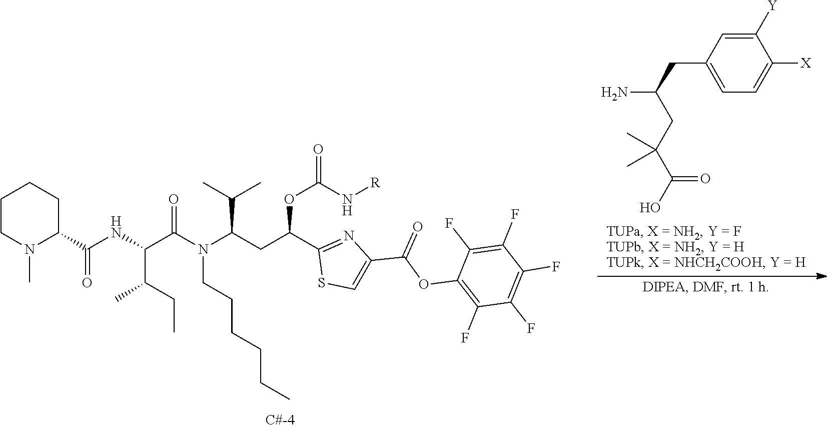

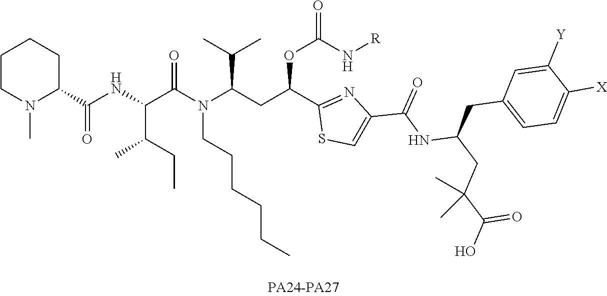

| Payload No. | R | X | Y |
|---|---|---|---|
| PA24 | OCONHCH₂CH₂OH | NH₂ | F |
| PA25 | OCONHCH₂(CH₂OCH₂)₂CH₂OH | NH₂ | F |
| PA26 | OCONHCH₂CH(OH)CH₂OH | NH₂ | H |
| PA27 | OCONHCH₂CH(OH)CH₂OH | NHCH₂CH₂OH | H |

Index of Reported Materials

| | | | |
|---|---|---|---|
| Code | Structures | CAS | References |

| | | | |
|---|---|---|---|
| 1-1a | 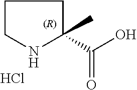 | 1508261-86-6 | Commercial (Accela) |
| 1-1b | 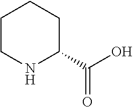 | 1723-00-8 | Commercial (Accela) |
| 4-1 | 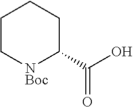 | 28697-17-8 | Commercial (Accela) |
| 7-8 | 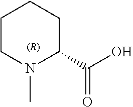 | 41447-17-0 | Commercial (Accela) |
| 9-1 | 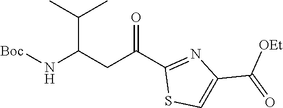 | 944559-47-1 | WO2020132658 |
| 7-1 | 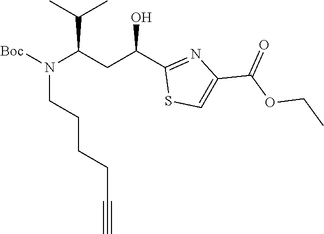 | 2757058-18-5 | WO2021262910 |
| 1-3a | 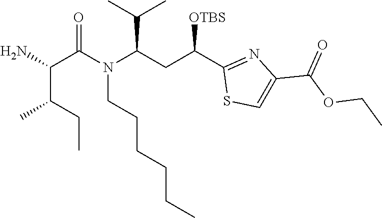 | 2757058-16-3 | WO2021262910 |
| 1-3b | 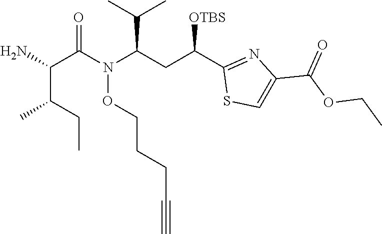 | 2447075-08-1 | WO2020132658 |
| 2-2 | 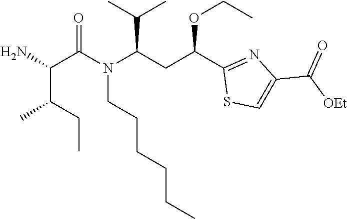 | 2757058-23-2 | WO2021262910 |
| 1-4 | 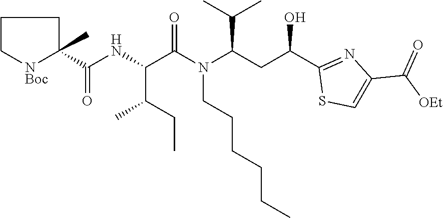 | 2757058-39-0 | WO2021262910 |
| 8-1 | 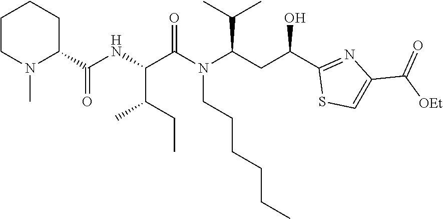 | 2757058-38-9 | WO2021262910 |
| 1-7 | 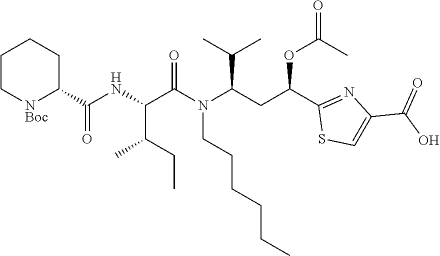 | 2757058-73-2 | WO2021262910 |
| 10-1 | 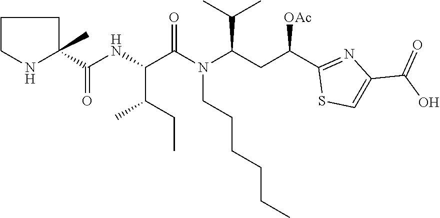 | 2757059-11-1 | WO2021262910 |
| 1-8a | 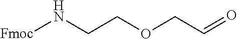 | 952661-22-2 | J. Org. Chem. 2007, 72, 7222- 7228 |
| 1-8b | 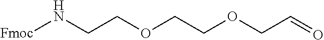 | 2113669-62-6 | Bioconj. Chem. 2017, 28, 2284- 2292 |
| 5-4a | 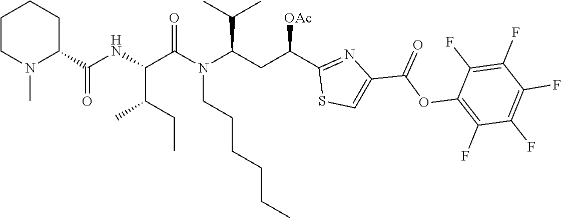 | 2447074-45-3 | WO2020132658 |
| 5-4b | 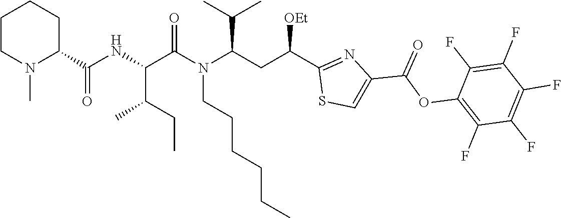 | 2447074-45-3 | WO2020132658 |
| TupA | 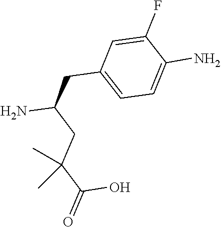 | | WO2020132658 |
| TupB | 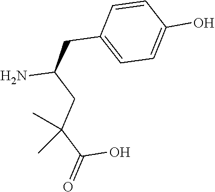 | 2356469-17-3 | WO2020132658 |
| TupC | 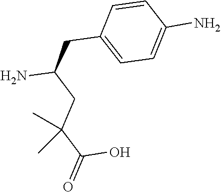 | 1924599-55-2 | US20160130299 |
| Boc-TupC | 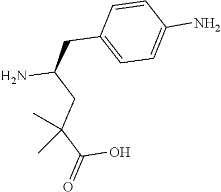 | 2447073-74-5 | WO2020132658 |
| Fmoc- Boc-TupC | 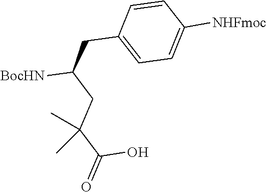 | 2447073-75-6 | WO2020132658 |
| Fmoc- TupD | 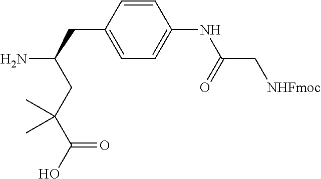 | 2447074-61-3 | WO2020132658 |
| Boc-TupD | 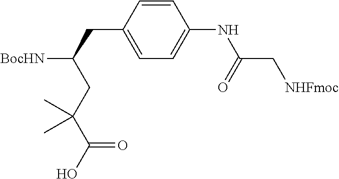 | 2447074-58-8 | WO2020132658 |
| Fmoc- vcPABC- PNP | 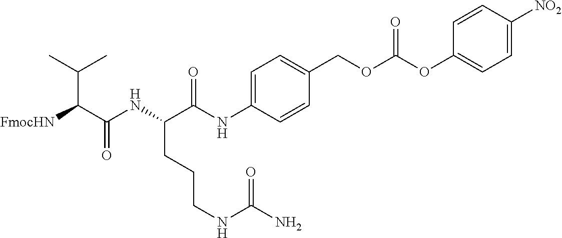 | 863971-53-3 | Commercial (MCE) |
| DIBAC- OSu | 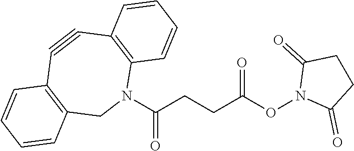 | 1353016-71-3 | Commercial (MCE) |
| DIBAC- PEG4- OSu | 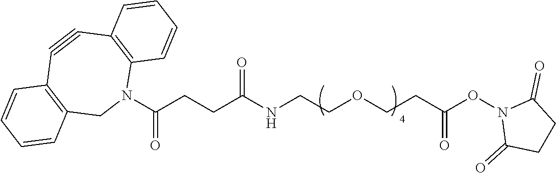 | 1427004-19-0 | Commercial (MCE) |
| COT- GGG-OH | 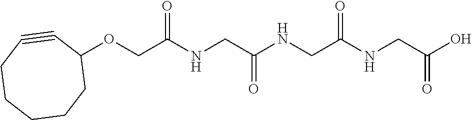 | 2504011-15-6 | WO2020146541 |
| 12-6 | 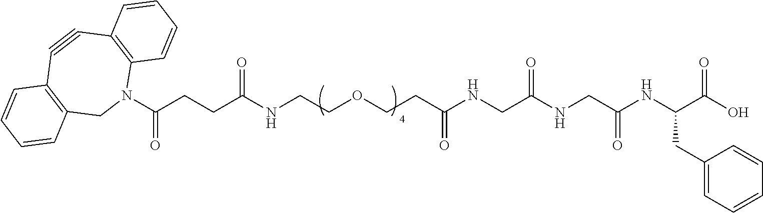 | 2361616-78-4 | WO2020146541 |
| 13-1 | 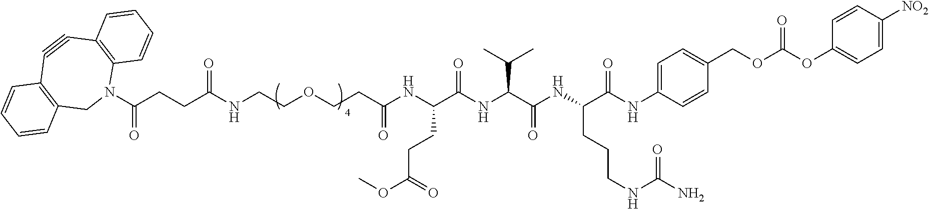 | 2454352-34-0 | WO2020146541 |
| 16-1 | 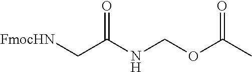 | 1599440-06-8 | Commercial (Accela) |

### Alternative General Procedures

### Alternative General Procedure I: Reductive-Amination of Alkyl-Modification on Mep

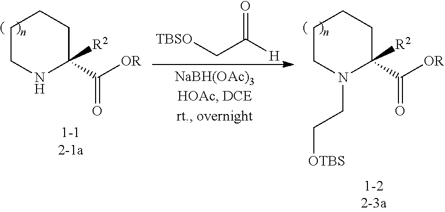

To a suspension of secondary-amine 1-1 or 2-la (1.0 equiv) and (tert-butyldimethylsilyloxy)acetaldehyde (1.0 equiv) in DCE (50 mM) was added acetic acid (0.1 equiv) and sodium triacetoxyborohydride (3.0 equiv) successively. The mixture was stirred at room temperature overnight, which was monitored by LCMS. The resulting mixture was filtered through a pad of celite and the filtrate was concentrated in vacuo. The residue was purified by reversed phase flash chromatography (0-50% acetonitrile in aq. TFA (0.01%)) to give compound 1-2 or 2-3a (29% yield) as a pale yellow solid which was sensitive to moisture.

### Alternative General Procedure II: Amidation of Tuv With Mep

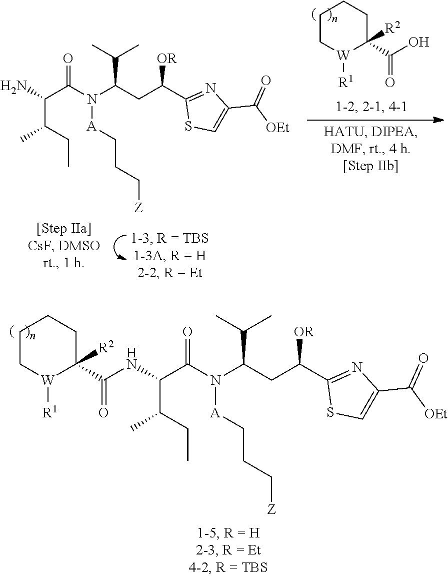

### Step IIa: Removal of TBS

A mixture of compound 1-3 (1.0 equiv) and CsF (2.0 equiv) in DMSO (0.5 M) was stirred at room temperature for an hour, which was monitored by LCMS. The resulting mixture was purified directly by reversed phase flash chromatography (0-60% acetonitrile in aq. TFA (0.01%)) to give de-TBS product as a white solid.

### Step IIb: Amidation Coupling

A solution of acid (modified Mep, 2.0 equiv) and HATU (2.0 equiv) in DMF (0.1 M) was stirred at room temperature for half an hour before the addition of the solution of the amine (modified Tuv) in DMF (1.0 equiv) obtained above and DIPEA (3.0 equiv). The resulting reaction mixture was stirred at room temperature for four hours, which was monitored by LCMS. The resulting mixture was purified directly by reversed phase flash chromatography (0-100% methanol in aq. ammonium bicarbonate (10 mM)) to give the amide as a white solid.

### Alternative General Procedure III: Hydrolysis of Ester to Acid

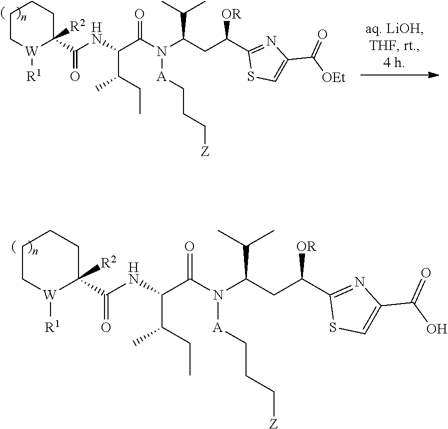

To a solution of the ester (1 equiv) in THF (20 mM) was added aq. lithium hydroxide (0.1 M, same volume as THF), and the reaction mixture was stirred at 25° C. for four hours, which was monitored by LCMS. The reaction mixture was quenched with cold aq. hydrochloride (1.0 M) until pH<7, and was then extracted with ethyl acetate. The combined organic solution was washed with brine, dried over anhydrous sodium sulfate, filtered, and concentrated in vacuo to give hydrolysis product 1-9A or 2-4A, which was then dissolved in pyridine (10 mg/mL). To the solution was added acetic anhydride (5.0 equiv) and the reaction mixture was stirred at 25° C. for four hours, which was monitored by LCMS. The resulting mixture was concentrated in vacuo, and the residue was purified by reversed phase flash chromatography (0-20% acetonitrile in aq. ammonium bicarbonate (0.8 g/L)) to give an acid as a white solid.

### Alternative General Procedure IV: Acetylation on Tuv

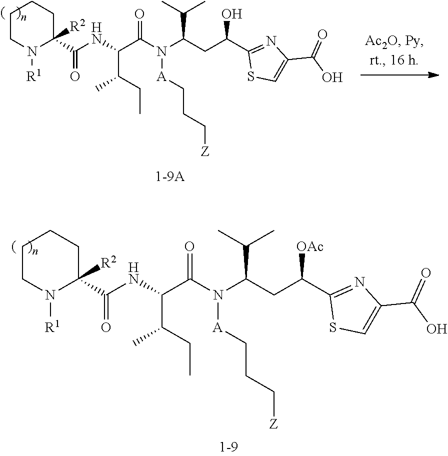

The hydrolysis product 1-9A obtained above was dissolved in pyridine (10 mg/mL). To the solution was added acetic anhydride (5.0 equiv) and the reaction mixture was stirred at 25° C. for four hours, which was monitored by LCMS. The resulting mixture was concentrated in vacuo, and the residue was purified by reversed phase flash chromatography (0-20% acetonitrile in aq. ammonium bicarbonate (0.8 g/L)) to give compound 1-9 as a white solid.

### Alternative General Procedure IVb: Reductive-amination of 1-7 to Synthesize 1-9

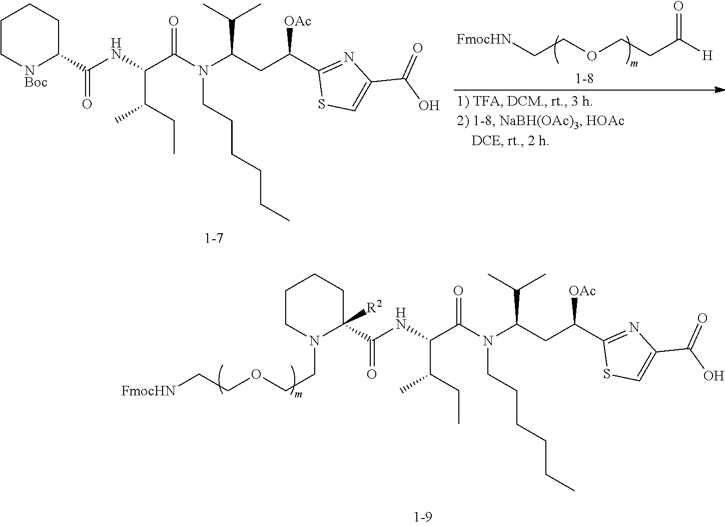

To a solution of compound 1-7 (1.0 equiv, synthesized as described in WO 2021262910) in DCM (0.25 M) was added TFA (1/3 volume of DCM), and the reaction mixture was stirred at 25° C. for 3 hrs until Boc was totally removed according to LCMS. The volatiles were removed in vacuo and the residue was purified by prep-HPLC (0-30% acetonitrile in aq. ammonium bicarbonate (0.8 g/L)) to give a de-Boc product as a white solid, which was then dissolved in DCE (10 mM). To the solution was added aldehyde 1-8 (1.0 equiv) and triacetoxyborohydride (3.0 equiv) and the reaction mixture was stirred at room temperature for an hour, which was monitored by LCMS. The mixture was quenched and washed with sat. aq. sodium bicarbonate and brine. The organic layer was dried over anhydrous sodium sulfate, filtered, and concentrated in vacuo. The crude product was purified by flash chromatography (silica gel, 0-100% ethyl acetate in petroleum ether) to give compound 1-9e or 1-9f (63-84% yield) as a white solid.

### Alternative General Procedure V: Synthesis of PFP Active Ester

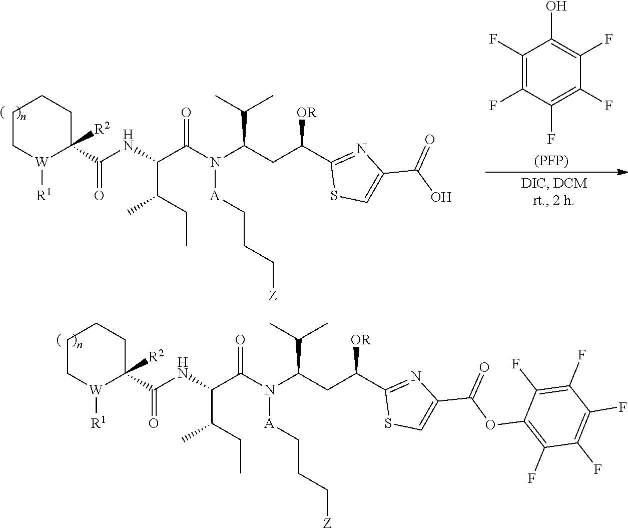

To a solution of the acid (1.0 equiv) in DCM (5 mL) was added pentafluorophenol (PFP) (2.0 equiv) and N,N-diisopropylcarbodiimide (DIC) (2.0 equiv) and the reaction mixture was stirred at 25° C. for two hours, which was monitored by LCMS. The resulting mixture was concentrated in vacuo to give crude PFP active ester as an oil, which was used in the next step without further purification.

### Alternative General Procedure VI: Amidation of Tup With PFP Ester

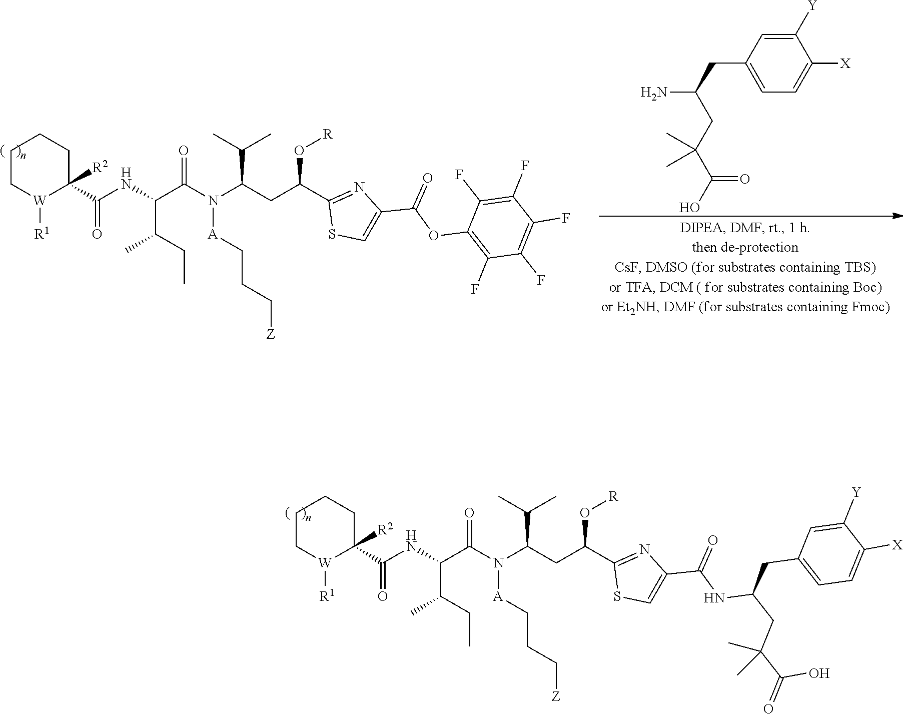

To a solution of intermediate PFP ester obtained above (1.0 equiv) in DMF (25-30 mM) was added Tup (1.0-2.0 equiv) and DIPEA (3.0 equiv) and the reaction mixture was stirred at 25° C. for four hours until starting materials were totally consumed, as monitored by LCMS. The resulting mixture was concentrated in vacuo to remove most of the DIPEA and the residue was directly purified by reversed phase flash chromatography (5-30% acetonitrile in aq. ammonium bicarbonate (0.8 g/L)) to give protected payloads as off-white-solids.

For Intermediates with TBS: To a solution of the TBS-protected payload provided above in DMSO (15 mM) was added cesium fluoride (3.0 equiv.), and the reaction mixture was stirred at 25° C. for two hours, which was monitored by LCMS. The resulting mixture was directly purified by prep-HPLC (5-95% acetonitrile in aq. ammonium bicarbonate (10 mM)) to give amides as white solids.

For Intermediates with Boc: To a solution of the Boc-protected payload provided above in DCM (5 mM) was added TFA (1/3 volume of DCM), and the mixture was stirred at 25° C. for four hours until Boc was totally removed, as monitored by LCMS. The resulting mixture was concentrated in vacuo and the residue was purified by prep-HPLC (5-95% acetonitrile in aq. formic acid (0.1%)) to give amides as white solids.

For Intermediates with Fmoc: To a solution of the Fmoc-protected payload provided above in DMF (10 mM) was added diethylamine (3.0 equiv), and the mixture was stirred at room temperature for two to sixteen hours, which was monitored by LCMS. The resulting mixture was directly purified by reversed phase flash chromatography (0-100% acetonitrile in aq. ammonium bicarbonate (0.05%)) to give amides as white solids.

### Alternative General Procedure VII: Reductive-amination on Tup to Provide Alkyl-Tup

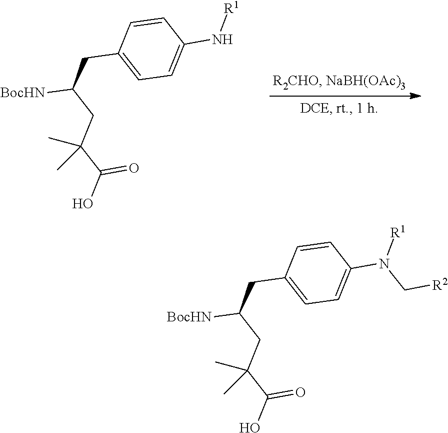

To a suspension of intermediate Tup (1.0 equiv) and corresponding aldehyde (1.2 equiv) in DCE (10 mL) was added sodium triacetoxyborohydride (1.5 equiv), and the reaction mixture was stirred at room temperature for an hour, which was monitored by LCMS. The resulting mixture was successively washed with aq. HCl (1 N), water, aq. sodium carbonate (10%), and brine, dried over anhydrous sodium sulfate, filtered, and concentrated in vacuo. The crude product was purified by reversed phase flash chromatography (0-100% acetonitrile in aq. TFA (0.01%)) to give alkyl Tup-aniline as a white solid.

### Alternative General Procedure VIII: Synthesis of Carbamates on Tuv

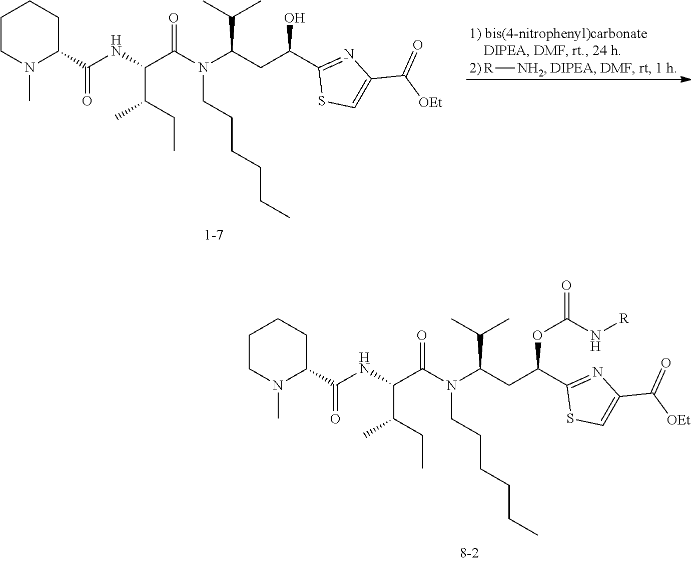

To a solution of alcohol 8-1 (1.0 equiv) in DMF (25 mM) was added DIPEA (5.0 equiv) and bis(4-nitrophenyl)carbonate (5.0 equiv), and the mixture was stirred at room temperature for twenty-four hours. To the resulting mixture was added a corresponding amine (1.0 equiv) and DIPEA (3.0 equiv), and the reaction mixture was stirred at room temperature for an hour and monitored by LCMS. The resulting mixture was directly purified by reversed phase flash chromatography (0-100% acetonitrile in aq. TFA (0.01%)) to give carbamate 8-2 as a white solid.

Scheme 1. Synthetic Route for Tubulysins Modified on Mep (PA-3, 4, 5, 7, 8, 9, 14,
and 15, aka PA1, PA2, PA3, PA6, PA7, PA8, PA11, and PA12, respectively)

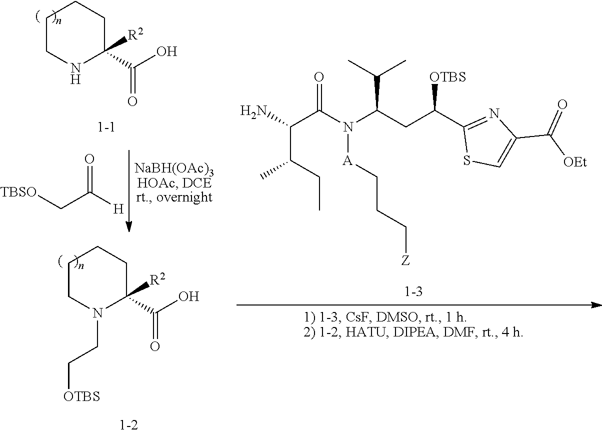

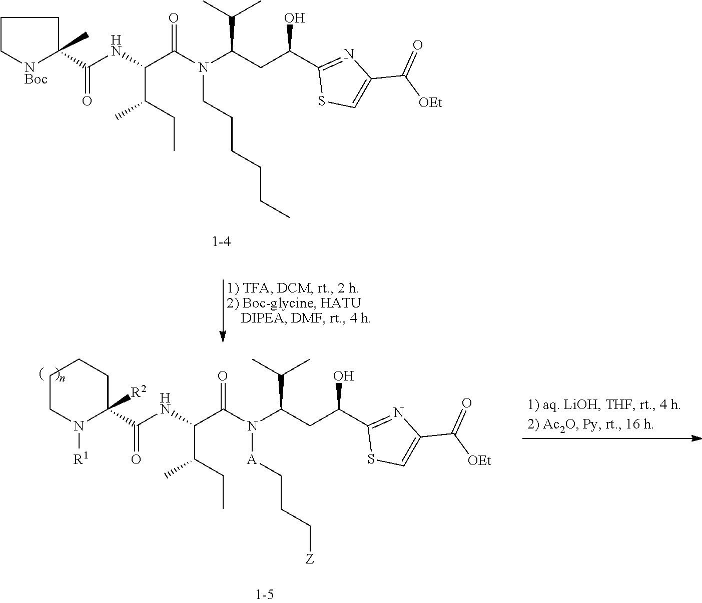

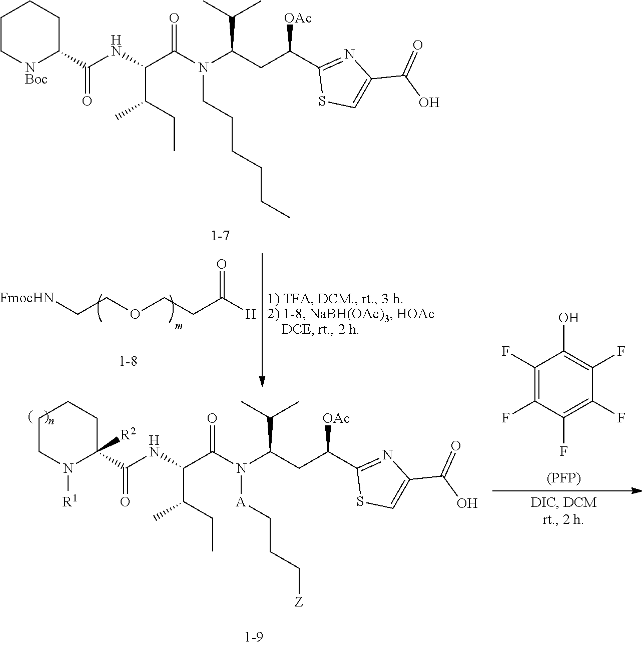

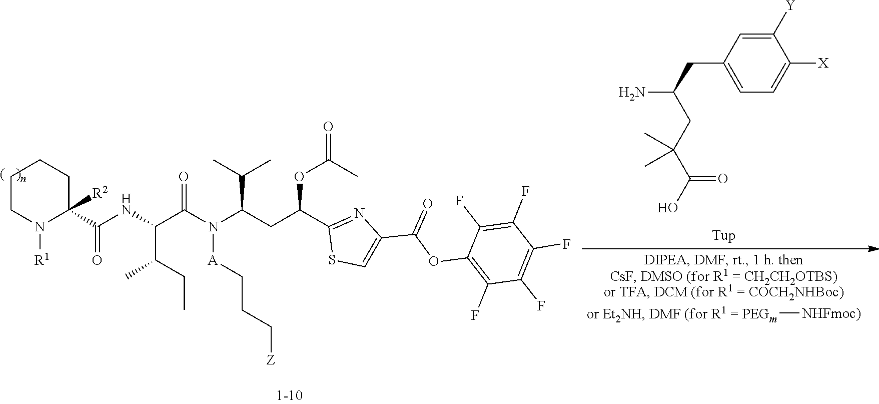

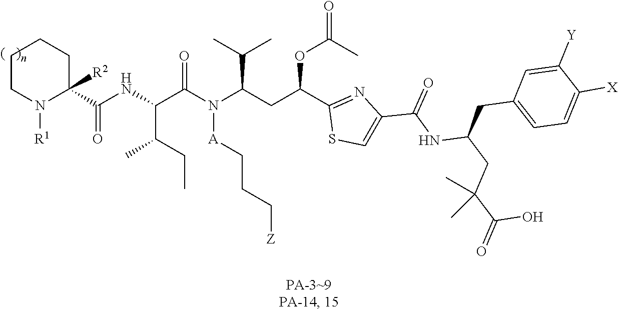

| | N/R² | R¹ | A/Z | X | Y |
|---|---|---|---|---|---|
| PA-3 aka PA1 | 0/Me | COCH₂NH₂ | CH₂/Et | NH₂ | F |
| PA-4 aka PA2 | 0/Me | COCH₂NH₂ | CH₂/Et | OH | H |
| PA-5 aka PA3 | 0/Me | CH₂CH₂OH | O/C≡CH | NH₂ | H |
| PA-7 aka PA6 | 0/Me | CH₂CH₂OH | CH₂/Et | NH₂ | H |
| PA-8 aka PA7 | 1/H | CH₂CH₂OH | CH₂/Et | NH₂ | H |
| PA-9 aka PA8 | 1/H | CH₂CH₂OH | CH₂/Et | NH₂ | F |
| PA-14 aka PA11 | 1/H | CH₂(CH₂OCH₂)NH₂ | CH₂/Et | NH₂ | H |
| PA-15 aka PA12 | 1/H | CH₂(CH₂OCH₂)₂NH₂ | CH₂/Et | NH₂ | H |

### Synthesis of Intermediate 1-2

### (2R)-1-{2-[(tert-butyldimethylsilyl)oxy]ethyl}-2-methylpyrrolidine-2-carboxylic acid (1-2a)

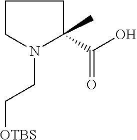 [SMILES: CC(C)(C)[Si](C)(C)OCCN1CCC[C@]1(C)C(=O)O]

Following Alternative General Procedure I from compound 1-la (1.0 g, 6.1 mmol), compound 1-2a (0.50 g, 29% yield) was obtained as a pale yellow solid. ESI m/z 288.3 (M+H)⁺. ¹H NMR (400 MHz, DMSO-d₆) δ 3.78-3.61 (m, 2H), 3.54-3.29 (m, 1H), 3.08-3.01 (m, 1H), 2.97-2.83 (m, 2H), 2.21-2.13 (m, 1H), 1.91-1.83 (m, 1H), 1.80-1.66 (m, 2H), 1.96-1.80 (m, 2H), 0.95-0.78 (m, 9H), 0.07 (s, 5H), 0.04 (s, 1H) ppm.

### (2R)-1-{2-[(tert-butyldimethylsilyl)oxy]ethyl}piperidine-2-carboxylic acid (1-2b)

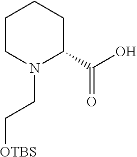 [SMILES: CC(C)(C)[Si](C)(C)OCCN1CCCC[C@@H]1C(=O)O]

Following Alternative General Procedure I from 1-1b (0.56 g, HCl salt, 4.4 mmol), compound 1-2b (0.16 g, 13% yield) was obtained as a white solid. ESI m/z 288.3 (M+H)⁺. ¹H NMR (400 MHz, DMSO-d₆) δ 3.76-3.72 (m, 2H), 3.21-3.16 (m, 1H), 3.15-3.10 (m, 1H), 2.90-2.81 (m, 1H), 2.73-2.66 (m, 1H), 2.63-2.53 (m, 1H), 1.84-1.74 (m, 1H), 1.72-1.61 (m, 1H), 1.58-1.45 (m, 3H), 1.41-1.30 (m, 1H), 0.89-0.83 (m, 9H), 0.06-0.02 (m, 6H) ppm.

### Synthesis of Intermediate 1-5

### Ethyl 2-[(1R,3R)-3-[(2S,3S)-2-{[(2R)-1-(2-{[(tert-butoxy)carbonyl]amino}acetyl)-2-methylpyrrolidin-2-yl]formamido}-N-hexyl-3-methylpentanamido]-1-hydroxy-4-methylpentyl]-1,3-thiazole-4-carboxylate (1-5a)

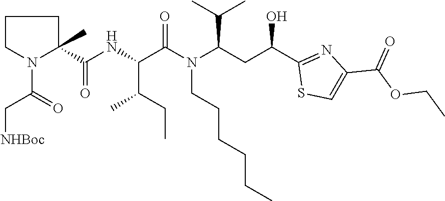 [SMILES: CCCCCCN(C(=O)[C@@H](NC(=O)[C@@]1(C)CCCN1C(=O)CNC(=O)OC(C)(C)C)[C@@H](C)CC)[C@H](C[C@@H](O)c1nc(C(=O)OCC)cs1)C(C)C]

To a solution of compound 1-4 (52 mg, 76 synthesized as described in WO 2021262910) in DCM (3 mL) was added TFA (1 mL), and the mixture was stirred at 25° C. for two hours until Boc was totally removed, as monitored by LCMS. The resulting mixture was concentrated in vacuo and the crude de-Boc product was dissolved in DMF (3 mL). To the solution was added HATU (29 mg, 76 μmol) and DIPEA (28 mg, 76 μmol), and the mixture was stirred at ° C. for ten minutes before the addition of Boc-glycine (40 mg, 69 μmol). The reaction mixture was then stirred at 25° C. for four hours, which was monitored by LCMS. The resulting mixture was purified by reversed phase flash chromatography (0-30% acetonitrile in aq. ammonium bicarbonate (0.8 g/L)) to give compound 1-5a (45 mg, 80% yield) as a white solid. ESI: 738.5 (M+H)⁺.

### Ethyl 2-[(1R,3R)-3-[(2S,3S)-2-{[(2R)-1-{2-[(tert-butyldimethylsilyl)oxy]ethyl}-2-methylpyrrolidin-2-yl]formamido}-3-methyl-N-(pent-4-yn-1-yloxy)pentanamido]-1-hydroxy-4-methylpentyl]-1,3-thiazole-4-carboxylate (1-5b)

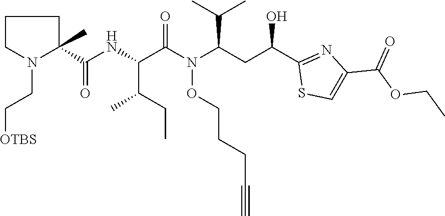 [SMILES: C#CCCCON(C(=O)[C@@H](NC(=O)[C@@]1(C)CCCN1CCO[Si](C)(C)C(C)(C)C)[C@@H](C)CC)[C@H](C[C@@H](O)c1nc(C(=O)OCC)cs1)C(C)C]

Following Alternative General Procedure II from 1-2a (0.15 g, 0.53 mmol) and 1-3b (0.18 g, 0.30 mmol), compounds 1-5b (0.10 g, 45% yield from 1-3b) and the de-TBS byproduct (54 mg, 32% yield) were obtained separately as white solids. Compound 1-5b: ESI m/z 737.2 (M+H)⁺. ¹H NMR (400 MHz, DMSO-d₆) δ 8.43 (s, 1H), 7.77 (d, J=10.0 Hz, 1H), 6.41 (d, J=6.4 Hz, 1H), 4.73-4.68 (m, 1H), 4.64-4.59 (m, 1H), 4.28 (q, J=7.2 Hz, 2H), 4.19-4.11 (m, 2H), 4.05-3.96 (m, 1H), 3.76-3.63 (m, 2H), 3.21-3.16 (m, 1H), 2.84 (t, J=2.0 Hz, 1H), 2.68-2.58 (m, 1H), 2.45-2.32 (m, 5H), 2.00-1.93 (m, 1H), 1.86-1.74 (m, 5H), 1.71-1.58 (m, 2H), 1.55-1.43 (m, 2H), 1.30 (t, J=7.2 Hz, 3H), 1.08 (s, 3H), 1.10-1.02 (m, 1H), 0.91-0.81 (m, 21H), 0.06 (s, 6H) ppm.

### Ethyl 2-[(1R,3R)-3-[(2S,3S)-2-{[(2R)-1-{2-[(tert-butyldimethylsilyl)oxy]ethyl}-2-methylpyrrolidin-2-yl]formamido}-N-hexyl-3-methylpentanamido]-1-hydroxy-4-methylpentyl]-1,3-thiazole-4-carboxylate (1-5c)

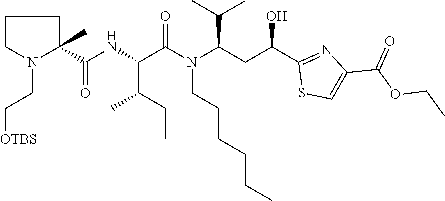 [SMILES: CCCCCCN(C(=O)[C@@H](NC(=O)[C@@]1(C)CCCN1CCO[Si](C)(C)C(C)(C)C)[C@@H](C)CC)[C@H](C[C@@H](O)c1nc(C(=O)OCC)cs1)C(C)C]

Following Alternative General Procedure II from 1-2a (64 mg, 0.23 mmol) and 1-3a (42 mg, 72 μmol), compound 1-5c (28 mg, 53% yield from 1-3a) was obtained as a white solid. ESI m/z 739.6 (M+H)⁺.

Ethyl 2-[(1R,3R)-3-[(2S,3S)-2-{[(2R)-1-{2-[(tert-butyldimethylsilyl)oxy]ethyl}piperidin-2-yl]formamido}-N-hexyl-3-methylpentanamido]-1-hydroxy-4-methylpentyl]-1,3-thiazole-4-carboxylate (1-5d)

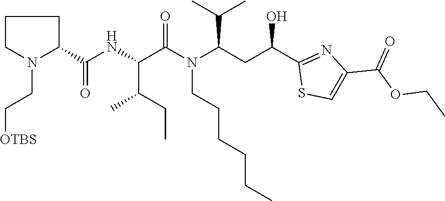 [SMILES: CCCCCCN(C(=O)[C@@H](NC(=O)[C@H]1CCCN1CCO[Si](C)(C)C(C)(C)C)[C@@H](C)CC)[C@H](C[C@@H](O)c1nc(C(=O)OCC)cs1)C(C)C]

Following Alternative General Procedure II from 1-2b (20 mg, 70 μmol) and 1-3a (38 mg, 65 μmol), compound 1-5d (30 mg, 62% yield from 1-3a) was obtained as a white solid. ESI m/z 739.6 (M+H)⁺.

### Synthesis of Intermediate 1-9

### 2-[(1R,3R)-1-(acetyloxy)-3-[(2S,3S)-2-{[(2R)-1-(2-{[(tert-butoxy)carbonyl]amino}acetyl)-2-methylpyrrolidin-2-yl]formamido}-N-hexyl-3-methylpentanamido]-4-methylpentyl]-1,3-thiazole-4-carboxylic acid (1-9a)

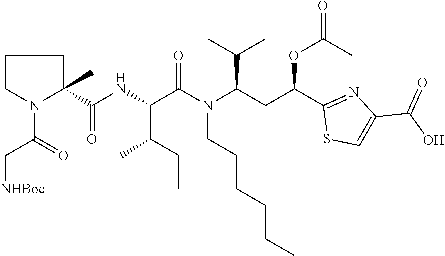 [SMILES: CCCCCCN(C(=O)[C@@H](NC(=O)[C@@]1(C)CCCN1C(=O)CNC(=O)OC(C)(C)C)[C@@H](C)CC)[C@H](C[C@@H](OC(C)=O)c1nc(C(=O)O)cs1)C(C)C]

Following Alternative General Procedures III and IVa from 1-5a (45 mg, 61 μmol), compound 1-9a (40 mg, 87% yield) was obtained as a white solid. ESI m/z 752.5 (M+H)⁺.

### 2-[(1R,3R)-1-(acetyloxy)-3-[(2S,3S)-2-{[(2R)-1-{2-[(tert-butyldimethylsilyl)oxy]ethyl}-2-methylpyrrolidin-2-yl]formamido}-3-methyl-N-(pent-4-yn-1-yloxy)pentanamido]-4-methylpentyl]-1,3-thiazole-4-carboxylic acid (1-9b)

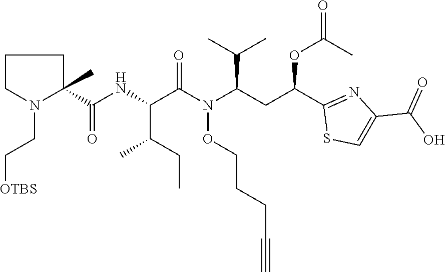 [SMILES: C#CCCCON(C(=O)[C@@H](NC(=O)[C@@]1(C)CCCN1CCO[Si](C)(C)C(C)(C)C)[C@@H](C)CC)[C@H](C[C@@H](OC(C)=O)c1nc(C(=O)O)cs1)C(C)C]

Following Alternative General Procedures III and IVa from 1-5b (0.10 g, 0.14 mmol), compound 1-9b (98 mg, 72% yield) was obtained as a white solid. ESI m/z 751.2 (M+H)⁺. ¹H NMR (400 MHz, DMSO-d₆) δ 13.10 (s, 1H), 8.44 (s, 1H), 7.78 (d, J=10.0 Hz, 1H), 5.75 (d, J=Hz, 1H), 4.75-4.70 (m, 1H), 4.19-4.13 (m, 1H), 4.07-3.98 (m, 2H), 3.76-3.63 (m, 2H), 3.19 (t, J=7.2 Hz, 1H), 2.86 (t, J=2.4 Hz, 1H), 2.68-2.59 (m, 1H), 2.45-2.29 (m, 6H), 2.10 (s, 3H), 2.03-1.97 (m, 1H), 1.84-1.69 (m, 5H), 1.62-1.54 (m, 2H), 1.50-1.43 (m, 1H), 1.09 (s, 3H), 1.06-0.99 (m, 1H), 0.95 (d, J=6.4 Hz, 3H), 0.90-0.83 (m, 18H), 0.06 (s, 6H) ppm.

### 2-[(1R,3R)-1-(acetyloxy)-3-[(2S,3S)-2-{[(2R)-1-{2-[(tert-butyldimethylsilyl)oxy]ethyl}-2-methylpyrrolidin-2-yl]formamido}-N-hexyl-3-methylpentanamido]-4-methylpentyl]-1,3-thiazole-4-carboxylic acid (1-9c)

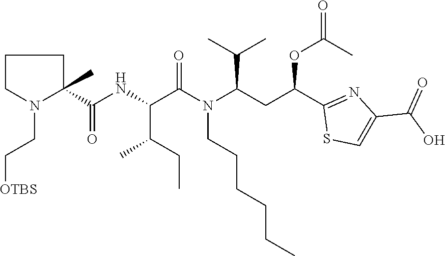 [SMILES: CCCCCCN(C(=O)[C@@H](NC(=O)[C@@]1(C)CCCN1CCO[Si](C)(C)C(C)(C)C)[C@@H](C)CC)[C@H](C[C@@H](OC(C)=O)c1nc(C(=O)O)cs1)C(C)C]

Following Alternative General Procedures III and IVa from 1-5c (78 mg, 0.10 mmol), compound 1-9c (50 mg, 62% yield) was obtained as a white solid. ESI m/z 753.5 (M+H)⁺.

### 2-[(1R,3R)-1-(acetyloxy)-3-[(2S,3S)-2-{[(2R)-1-{2-[(tert-butyldimethylsilyl)oxy]ethyl}piperidin-2-yl]formamido}-N-hexyl-3-methylpentanamido]-4-methylpentyl]-1,3-thiazole-4-carboxylic acid (1-9d)

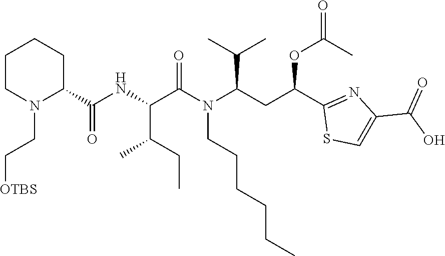 [SMILES: CCCCCCN(C(=O)[C@@H](NC(=O)[C@H]1CCCCN1CCO[Si](C)(C)C(C)(C)C)[C@@H](C)CC)[C@H](C[C@@H](OC(C)=O)c1nc(C(=O)O)cs1)C(C)C]

Following Alternative General Procedures III and IVa from 1-5d (30 mg, 41 μmol), compound 1-9d (19 mg, 64% yield) was obtained as a white solid. ESI m/z 753.5 (M+H)⁺.

### 2-[(1R,3R)-1-(acetyloxy)-3-[(2S,3S)-2-{[(2R)-1-{2-[2-({[(9H-fluoren-9-yl)methoxy]carbonyl}amino)ethoxy]ethyl}piperidin-2-yl]formamido}-N-hexyl-3-methylpentanamido]-4-methylpentyl]-1,3-thiazole-4-carboxylic acid (1-9e)

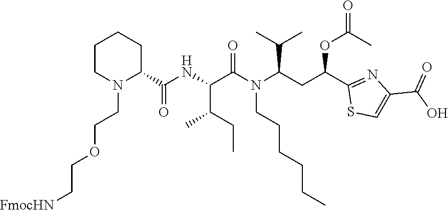

Following Alternative General Procedure IVb starting from compound 1-7 (71 mg, mmol) with 1-8a (m=1, 33 mg, 0.10 mmol, synthesized as described in J. Org. Chem. 2007, 72, 7222-7228), compound 1-9e (76 mg, 84% yield) was obtained as a white solid. ESI m/z 904.3 (M+H)⁺.

### 2-[(1R,3R)-1-(acetyloxy)-3-[(2S,3S)-2-{[(2R)-1-(2-{2-[2-({[(9H-fluoren-9-yl)methoxy]carbonyl}amino)ethoxy]ethoxy}ethyl)piperidin-2-yl]formamido}-N-hexyl-3-methylpentanamido]-4-methylpentyl]-1,3-thiazole-4-carboxylic acid (1-9f)

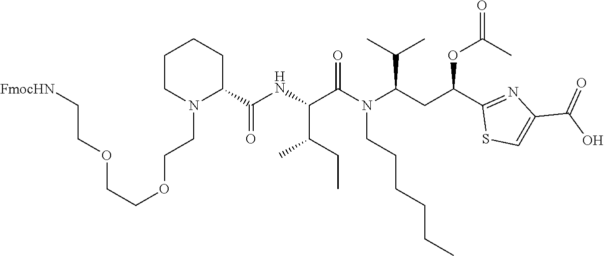

Following Alternative General Procedure IVb starting from compound 1-7 (29 mg, 42 μmol) with 1-8b (m=2, 16 mg, 42 μmol, synthesized as described in Bioconjugate Chemistry 2017, 28, 2284-2292), compound 1-9f (25 mg, 63% yield) was obtained as a white solid. ESI m/z 948.5 (M+H)⁺.

### Synthesis of Intermediate 1-10

2,3,4,5,6-Pentafluorophenyl 2-[(1R,3R)-1-(acetyloxy)-3-[(2S,3S)-2-{[(2R)-1-(2-{[(tert-butoxy)carbonyl]amino}acetyl)-2-methylpyrrolidin-2-yl]formamido}-N-hexyl-3-methylpentanamido]-4-methylpentyl]-1,3-thiazole-4-carboxylate (1-10a)

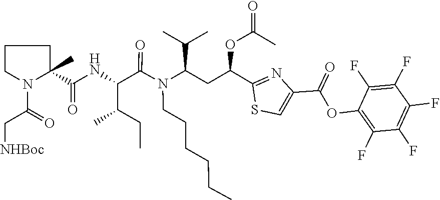 [SMILES: CCCCCCN(C(=O)[C@@H](NC(=O)[C@@]1(C)CCCN1C(=O)CNC(=O)OC(C)(C)C)[C@@H](C)CC)[C@H](C[C@@H](OC(C)=O)c1nc(C(=O)Oc2c(F)c(F)c(F)c(F)c2F)cs1)C(C)C]

Following Alternative General Procedure V starting from intermediate 1-9a (20 mg, 27 μmol), crude compound 1-10a (24 mg) was obtained as light yellow oil, which was used in the next step without purification. ESI m/z 918.4 (M+H)⁺.

### 2,3,4,5,6-Pentafluorophenyl 2-[(1R,3R)-1-(acetyloxy)-3-[(2S,3S)-2-{[(2R)-1-{2-[(tert-butyldimethylsilyl)oxy]ethyl}-2-methylpyrrolidin-2-yl]formamido}-3-methyl-N-(pent-4-yn-1-yloxy)pentanamido]-4-methylpentyl]-1,3-thiazole-4-carboxylate (1-10b)

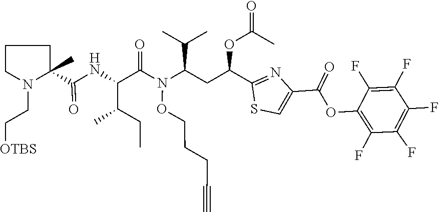 [SMILES: C#CCCCON(C(=O)[C@@H](NC(=O)[C@@]1(C)CCCN1CCO[Si](C)(C)C(C)(C)C)[C@@H](C)CC)[C@H](C[C@@H](OC(C)=O)c1nc(C(=O)Oc2c(F)c(F)c(F)c(F)c2F)cs1)C(C)C]

Following Alternative General Procedure V starting from intermediate 1-9b (60 mg, 80 μmol), crude compound 1-10b (100 mg) was obtained as light yellow oil, which was used in the next step without purification. ESI m/z 917.5 (M+H)⁺.

### 2,3,4,5,6-Pentafluorophenyl 2-[(1R,3R)-1-(acetyloxy)-3-[(2S,3S)-2-{[(2R)-1-{2-[(tert-butyldimethylsilyl)oxy]ethyl}-2-methylpyrrolidin-2-yl]formamido}-N-hexyl-3-methylpentanamido]-4-methylpentyl]-1,3-thiazole-4-carboxylate (1-10c)

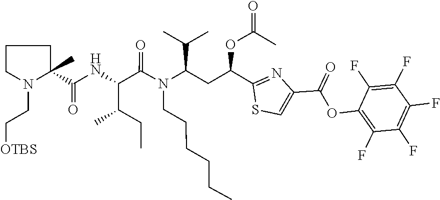 [SMILES: CCCCCCN(C(=O)[C@@H](NC(=O)[C@@]1(C)CCCN1CCO[Si](C)(C)C(C)(C)C)[C@@H](C)CC)[C@H](C[C@@H](OC(C)=O)c1nc(C(=O)Oc2c(F)c(F)c(F)c(F)c2F)cs1)C(C)C]

Following Alternative General Procedure V starting from intermediate 1-9c (38 mg, 51 μmol), crude compound 1-10c (46 mg) was obtained as light yellow oil, which was used in the next step without purification. ESI m/z 919.4 (M+H)⁺.

### 2,3,4,5,6-Pentafluorophenyl 2-[(1R,3R)-1-(acetyloxy)-3-[(2S,3S)-2-{[(2R)-1-{2-[(tert-butyldimethylsilyl)oxy]ethyl}piperidin-2-yl]formamido}-N-hexyl-3-methylpentanamido]-4-methylpentyl]-1,3-thiazole-4-carboxylate (1-10d)

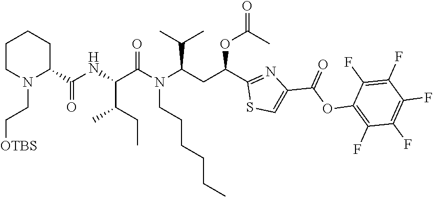 [SMILES: CCCCCCN(C(=O)[C@@H](NC(=O)[C@H]1CCCCN1CCO[Si](C)(C)C(C)(C)C)[C@@H](C)CC)[C@H](C[C@@H](OC(C)=O)c1nc(C(=O)Oc2c(F)c(F)c(F)c(F)c2F)cs1)C(C)C]

Following Alternative General Procedure V starting from intermediate 1-9d (19 mg, 25 μmol), crude compound 1-10d (23 mg) was obtained as a yellow oil, which was used in the next step without purification. ESI m/z 919.5 (M+H)⁺.

### 2,3,4,5,6-Pentafluorophenyl 2-[(1R,3R)-1-(acetyloxy)-3-[(2S,3S)-2-{[(2R)-1-{2-12-({[(9H-fluoren-9-yl)methoxy]carbonyl}amino)ethoxy]ethyl}piperidin-2-yl]formamido}-N-hexyl-3-methylpentanamido]-4-methylpentyl]-1,3-thiazole-4-carboxylate (1-10e)

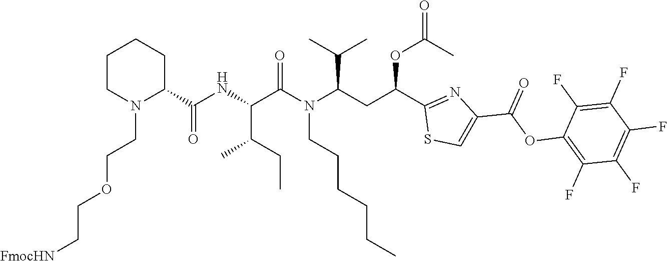

Following Alternative General Procedure V starting from intermediate 1-9e (76 mg, 84 μmol), crude compound 1-10e (90 mg) was obtained as a colorless oil, which was used in the next step without purification. ESI m/z 1070.5 (M+H)⁺.

### 2,3,4,5,6-Pentafluorophenyl 2-[(1R,3R)-1-(acetyloxy)-3-[(2S,3S)-2-{[(2R)-1-(2-{2-12-({[(9H-fluoren-9-yl)methoxy]carbonyl}amino)ethoxy]ethoxy}ethyl)piperidin-2-yl]formamido}-N-hexyl-3-methylpentanamido]-4-methylpentyl]-1,3-thiazole-4-carboxylate (1-10f)

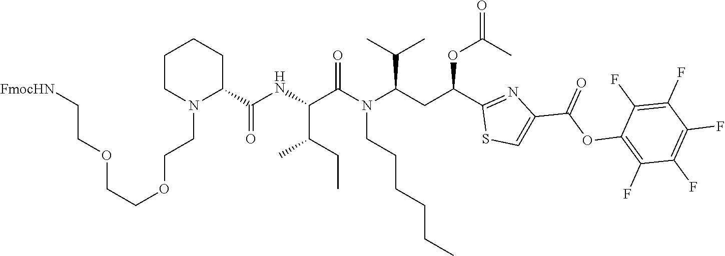

Following Alternative General Procedure V starting from intermediate 1-9f (25 mg, 26 μmol), crude compound 1-10f (29 mg) was obtained as a colorless oil, which was used in the next step without purification. ESI m/z 1114.5 (M+H)⁺.

### Synthesis of Payloads PA-3, PA-4, PA-5, PA-7, PA-8, PA-9, PA-14, and PA-15 PA-3 aka PA1, PA2, PA3, PA6, PA7, PA8, PA11, and PA12, Respectively

### (4S)-4-({2-[(1R,3R)-1-(acetyloxy)-3-[(2S,3S)-2-{[(2R)-1-(2-aminoacetyl)-2-methylpyrrolidin-2-yl]formamido}-N-hexyl-3-methylpentanamido]-4-methylpentyl]-1,3-thiazol-4-yl}formamido)-5-(4-amino-3-fluorophenyl)-2,2-dimethylpentanoic acid (PA-3 aka PA1)

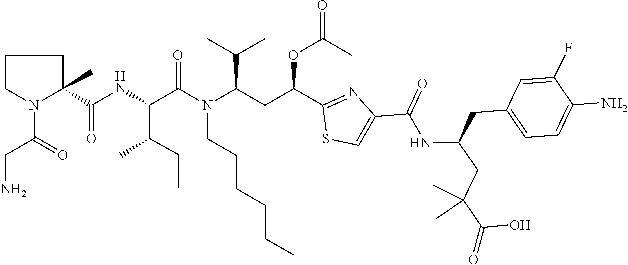 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following Alternative General Procedure VI (and then Boc removal) from 1-10a (24 mg, 27 μmol) with TupA (11 mg, 41 synthesized as described in WO 2020132658), payload PA-3 aka PA1 (3.8 mg, 16% yield from 1-9a) was obtained as a white solid. ESI m/z 888.5 (M+H)⁺. ¹H NMR (400 MHz, DMSO-d₆) δ 8.16 (s, 1H), 7.59 (d, J=8.8 Hz, 1H), 7.48 (d, J=9.6 Hz, 1H), 6.74 (d, J=12.8 Hz, 1H), 6.68-6.59 (m, 2H), 5.64 (d, J=13.2 Hz, 1H), 4.93 (s, 2H), 4.50 (t, J=9.6 Hz, 1H), 4.25-4.16 (m, 1H), 3.86-3.81 (m, 1H), 3.74-3.66 (m, 1H), 3.61-3.56 (m, 3H), 2.98-2.89 (m, 2H), 2.61 (d, J=6.0 Hz, 2H), 2.34-2.32 (m, 1H), 2.28-2.20 (m, 2H), 2.14 (s, 3H), 2.00-1.92 (m, 2H), 1.86-1.76 (m, 3H), 1.75-1.70 (m, 2H), 1.69-1.64 (m, 2H), 1.50 (s, 3H), 1.46-1.40 (m, 1H), 1.27-1.21 (m, 7H), 1.16-1.09 (m, 1H), 1.06 (s, 6H), 1.50 (d, J=6.4 Hz, 3H), 0.87-(m, 10H), 0.69-0.65 (d, J=5.2 Hz, 3H) ppm. ¹⁹F NMR (376 MHz, DMSO-d₆) δ −135.45 ppm.

### PA-4 aka PA2

### (4S)-4-({2-[(1R,3R)-1-(acetyloxy)-3-[(2S,3S)-2-{[(2R)-1-(2-aminoacetyl)-2-methylpyrrolidin-2-yl]formamido}-N-hexyl-3-methylpentanamido]-4-methylpentyl]-1,3-thiazol-4-yl}formamido)-5-(4-hydroxyphenyl)-2,2-dimethylpentanoic acid (PA-4 aka PA2)

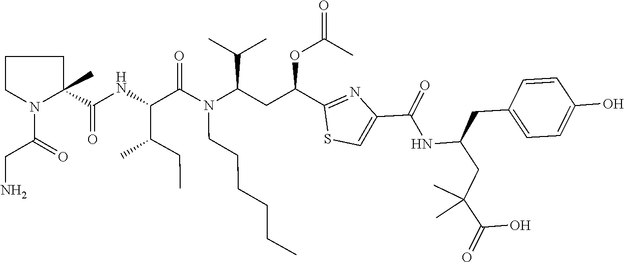 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(O)cc2)CC(C)(C)C(=O)O)cs1)C(C)C]

Following Alternative General Procedure VI (and then Boc removal) from 1-10a (24 mg, 27 μmol) with TupB (11 mg, 41 μmol, synthesized as described in WO 2020132658), payload PA-4 aka PA2 (6.7 mg, 28% yield from 1-9a) was obtained as a white solid. ESI m/z 871.7 (M+H)⁺. ¹H NMR (400 MHz, DMSO-d₆) δ 8.37 (s, 1H), 8.17 (s, 1H), 7.60-7.48 (m, 1H), 6.94 (d, J=7.6 Hz, 2H), 6.63 (d, J=8.0 Hz, 2H), 5.64 (d, J=11.6 Hz, 1H), 4.45 (t, J=9.6 Hz, 1H), 4.25 (s, 1H), 3.90-3.80 (m, 2H), 3.61-3.57 (m, 1H), 3.28-3.26 (m, 3H), 2.92 (t, J=9.6 Hz, 2H), 2.70-2.60 (m, 2H), 2.35-2.20 (m, 2H), 2.14 (s, 3H), 2.00-1.88 (m, 3H), 1.85-1.75 (m, 2H), 1.73-1.65 (m, 5H), 1.47 (s, 3H), 1.35-1.20 (m, 7H), 1.05 (s, 6H), 0.96 (d, J=6.0 Hz, 3H), 0.88-0.82 (m, 4H), (m, 7H), 0.67 (s, 3H) ppm.

### PA-5 aka PA3

### (4S)-4-({2-[(1R,3R)-1-(acetyloxy)-3-[(2S,3S)-2-{[(2R)-1-(2-hydroxyethyl)-2-methylpyrrolidin-2-yl]formamido}-3-methyl-N-(pent-4-yn-1-yloxy)pentanamido]-4-methylpentyl]-1,3-thiazol-4-yl}formamido)-5-(4-aminophenyl)-2,2-dimethylpentanoic acid (PA-5 aka PA3)

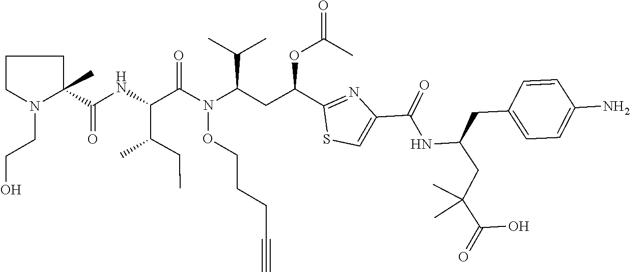 [SMILES: C#CCCCON(C(=O)[C@@H](NC(=O)[C@@]1(C)CCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following Alternative General Procedure VI (and then TBS removal) from 1-10b (100 mg, crude, 80 μmol calculated from 1-9b) with TupC (56 mg, 0.16 mmol, synthesized as described in U.S. 20160130299), payload PA-5 aka PA3 (30 mg, 37% yield from 1-9b) was obtained as a white solid. ESI m/z 855.4 (M+H)⁺. ¹H NMR (400 MHz, DMSO-d₆) δ 12.12 (s, 1H), 8.16 (s, 1H), 8.15 (d, J=10.0 Hz, 1H), 7.60 (d, J=9.6 Hz, 1H), 6.82 (d, J=8.0 Hz, 2H), 6.43 (d, J=8.0 Hz, 2H), 5.83 (d, J=10.8 Hz, 1H), 4.90-4.80 (m, 2H), 4.71 (t, J=8.8 Hz, 1H), 4.28-4.25 (m, 1H), 4.19-4.15 (m, 1H), 4.08-4.04 (m, 2H), 3.54-3.44 (m, 2H), 3.21 (d, J=6.8 Hz, 1H), 2.85 (t, J=2.0 Hz, 1H), 2.67-2.58 (m, 2H), 2.56-2.54 (m, 1H), 2.45-2.30 (m, 6H), 2.14 (s, 3H), 2.03-1.94 (m, 1H), 1.91-1.77 (m, 5H), 1.70-1.55 (m, 4H), 1.50-1.42 (m, 1H), 1.14-1.03 (m, 10H), 0.96 (d, J=6.4 Hz, 3H), 0.88 (d, J=6.4 Hz, 3H), 0.86-0.82 (m, 6H) ppm.

### PA-7 aka PA6

### (4S)-4-({2-[(1R,3R)-1-(acetyloxy)-3-[(2S,3S)—N-hexyl-2-{[(2R)-1-(2-hydroxyethyl)-2-methylpyrrolidin-2-yl]formamido}-3-methylpentanamido]-4-methylpentyl]-1,3-thiazol-4-yl}formamido)-5-(4-aminophenyl)-2,2-dimethylpentanoic acid (PA-7 aka PA6)

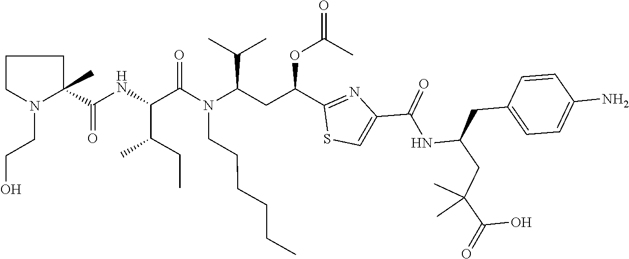 [SMILES: CCCCCCN(C(=O)[C@@H](NC(=O)[C@@]1(C)CCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following Alternative General Procedure VI (and then TBS removal) from 1-10c (46 mg, crude, 50 μmol calculated from 1-9c) with TupC (35 mg, 0.10 mmol), payload PA-7 aka PA6 (12 mg, 28% yield from 1-9c) was obtained as a white solid. ESI m/z 857.3 (M+H)⁺. ¹H NMR (400 MHz, DMSO-d₆) δ 8.22-8.14 (m, 2H), 6.80 (d, J=8.2 Hz, 2H), 6.44 (d, J=8.2 Hz, 2H), (m, 1H), 4.93-4.75 (m, 3H), 4.42 (t, J=9.7 Hz, 1H), 4.17 (dd, J=18.1, 6.7 Hz, 1H), 3.85-3.73 (m, 1H), 3.55-3.42 (m, 4H), 3.24-3.14 (m, 2H), 3.01-2.86 (m, 2H), 2.69-2.55 (m, 2H), 2.37-2.20 (m, 4H), 2.13 (s, 3H), 1.93-1.72 (m, 5H), 1.67-1.43 (m, 6H), 1.36-1.21 (m, 6H), 1.09 (s, 3H), 1.06-0.90 (m, 9H), 0.90-0.76 (m, 9H), 0.70-0.59 (m, 3H) ppm.

### PA-8 aka PA7

### (4S)-4-({2-[(1R,3R)-1-(acetyloxy)-3-[(2S,3S)—N-hexyl-2-{[(2R)-1-(2-hydroxyethyl)piperidin-2-yl]formamido}-3-methylpentanamido]-4-methylpentyl]-1,3-thiazol-4-yl}formamido)-5-(4-aminophenyl)-2,2-dimethylpentanoic acid (PA-8 aka PA7)

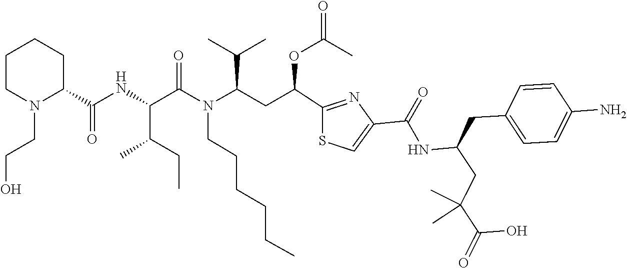 [SMILES: CCCCCCN(C(=O)[C@@H](NC(=O)[C@H]1CCCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following Alternative General Procedure VI (and then TBS removal) from 1-10d (23 mg, crude, 25 μmol calculated from 1-9d) with TupC (18 mg, 50 μmol), payload PA-8 aka PA7 (6.6 mg, 31% yield from 1-9d) was obtained as a white solid. ESI m/z 857.3 (M+H)⁺. ¹H NMR (500 MHz, DMSO-d₆) δ 8.16 (s, 1H), 7.74-7.66 (m, 1H), 7.55-7.46 (m, 1H), 6.80 (d, J=8.3 Hz, 2H), 6.44 (d, J=8.3 Hz, 2H), 5.68-5.62 (m, 1H), 4.92-4.78 (m, 2H), 4.50 (t, J=9.4 Hz, 1H), 4.25-4.16 (m, 1H), 3.74-3.65 (m, 1H), 3.56-3.45 (m, 1H), 3.11-2.95 (m, 3H), 2.83-2.74 (m, 1H), 2.65-2.59 (m, 1H), 2.57-2.54 (m, 1H), 2.39-2.34 (m, 1H), 2.31-2.16 (m, 3H), 2.14 (s, 3H), 2.07-1.96 (m, 2H), 1.91-1.79 (m, 3H), 1.79-1.41 (m, 8H), 1.39-1.19 (m, 10H), 1.15-1.03 (m, 6H), 1.00-0.90 (m, 3H), 0.89-0.79 (m, 9H), 0.72-0.66 (m, 3H) ppm.

### PA-9 aka PA8

### (4S)-4-({2-[(1R,3R)-1-(acetyloxy)-3-[(2S,3S)—N-hexyl-2-{[(2R)-1-(2-hydroxyethyl)piperidin-2-yl]formamido}-3-methylpentanamido]-4-methylpentyl]-1,3-thiazol-4-yl}formamido)-5-(4-amino-3-fluorophenyl)-2,2-dimethylpentanoic acid (PA-9 aka PA8)

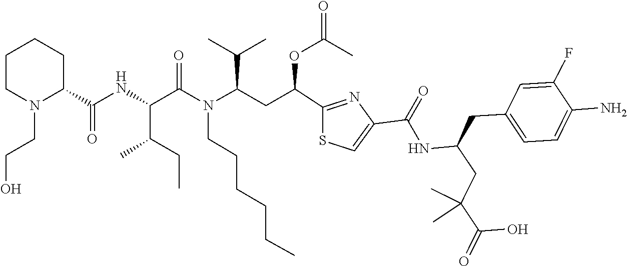 [SMILES: CCCCCCN(C(=O)[C@@H](NC(=O)[C@H]1CCCCN1CCO)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following Alternative General Procedure VI (and then TBS removal) from 1-10d (46 mg, crude, 50 μmol calculated from 1-9d) with TupA (18 mg, 50 μmol), payload PA-9 aka PA8 (9.5 mg, 22% yield from 1-9d) was obtained as a white solid. ESI m/z 875.4 (M+H)⁺. ¹H NMR (400 MHz, DMSO-d₆) δ 8.17 (s, 1H), 7.69 (d, J=9.5 Hz, 1H), 6.75 (dd, J=12.6, 1.2 Hz, 1H), 6.69-6.60 (m, 2H), 5.68-5.62 (m, 1H), 4.91 (s, 2H), 4.50 (t, J=9.4 Hz, 1H), 4.23-4.15 (m, 1H), 3.77-3.67 (m, 1H), 3.55-3.46 (m, 2H), 3.07-2.93 (m, 3H), 2.80-2.75 (m, 1H), 2.68-2.56 (m, 3H), 2.34-2.17 (m, 3H), 2.14 (s, 3H), 2.07-1.96 (m, 2H), 1.92-1.45 (m, 10H), 1.42-1.17 (m, 10H), 1.16-1.09 (m, 1H), 1.05 (d, J=4.2 Hz, 6H), 0.95 (d, J=6.4 Hz, 3H), 0.88-0.77 (m, 9H), 0.69 (d, J=6.2 Hz, 3H) ppm.

### PA-14 aka PAH

### (4S)-4-({2-[(1R,3R)-1-(acetyloxy)-3-[(2S,3S)-2-{[(2R)-1-[2-(2-aminoethoxy)ethyl]piperidin-2-yl]formamido}-N-hexyl-3-methylpentanamido]-4-methylpentyl]-1,3-thiazol-4-yl}formamido)-5-(4-aminophenyl)-2,2-dimethylpentanoic acid (PA-14 aka PA11)

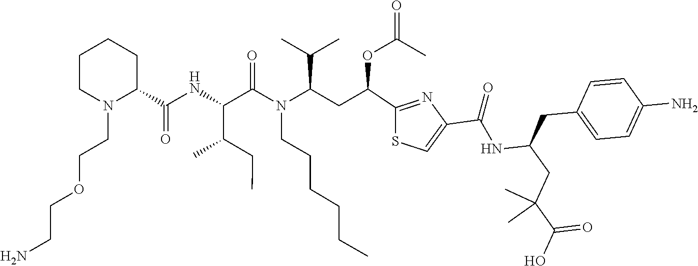 [SMILES: CCCCCCN(C(=O)[C@@H](NC(=O)[C@H]1CCCCN1CCOCCN)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following Alternative General Procedure VI (and then Fmoc removal) from 1-10e (90 mg, crude, 84 μmol calculated from 1-9e) with TupC (20 mg, 84 μmol), payload PA-14 aka PAH (44 mg, 57% yield from 1-9e) was obtained as a white solid. ESI m/z 901.5 (M+H)⁺. ¹H NMR (400 MHz, DMSO-d₆) δ 12.06 (s, 1H), 9.05 (d, J=7.2 Hz, 1H), 8.18 (s, 1H), 7.98-7.64 (m, 4H), 6.97 (d, J=7.9 Hz, 2H), 6.69 (d, J=6.3 Hz, 2H), 5.68-5.58 (m, 1H), 4.49 (t, J=9.0 Hz, 1H), 4.26-4.18 (m, 1H), 4.01-3.92 (m, 1H), 3.77-3.59 (m, 5H), 3.16-3.01 (m, 6H), 2.74-2.58 (m, 3H), 2.36-2.22 (m, 2H), 2.19-2.06 (m, 4H), 2.03-1.64 (m, 10H), 1.62-1.52 (m, 1H), 1.51-1.21 (m, 10H), 1.20-1.14 (m, 1H), 1.13-1.02 (m, 6H), 0.97 (d, J=6.4 Hz, 3H), 0.92-0.78 (m, 9H), 0.71 (d, J=5.6 Hz, 3H) ppm.

### PA-15 aka PA12

### (4S)-4-({2-[(1R,3R)-1-(acetyloxy)-3-[(2S,3S)-2-{[(2R)-1-{2-[2-(2-aminoethoxy)ethoxy]ethyl}piperidin-2-yl]formamido}-N-hexyl-3-methylpentanamido]-4-methylpentyl]-1,3-thiazol-4-yl}formamido)-5-(4-aminophenyl)-2,2-dimethylpentanoic acid (PA-15 aka PA12)

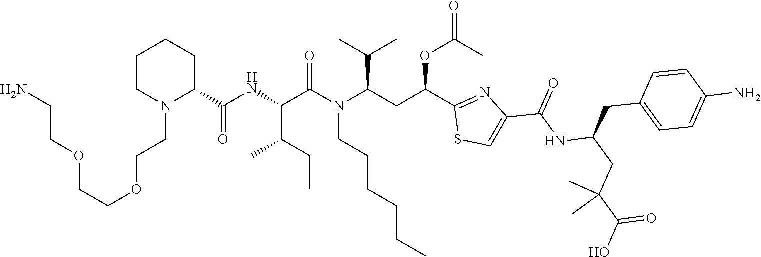 [SMILES: CCCCCCN(C(=O)[C@@H](NC(=O)[C@H]1CCCCN1CCOCCOCCN)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following Alternative General Procedure VI (and then Fmoc removal) from 1-10f (29 mg, crude, 26 μmol calculated from 1-90 with TupC (7.0 mg, 26 μmol), payload PA-15 aka PA12 (11 mg, 44% yield from 1-90 was obtained as a white solid. ESI m/z 945.3 (M+H)⁺. ¹H NMR (400 MHz, DMSO-d₆) δ 9.08 (d, J=5.6 Hz, 1H), 8.17 (s, 1H), 7.93-7.63 (m, 4H), 6.97 (br s, 2H), 6.68 (br s, 2H), 5.67-5.59 (m, 1H), 4.50 (t, J=9.3 Hz, 1H), 4.25-4.18 (m, 1H), 3.97-3.90 (m, 1H), 3.73-3.55 (m, 9H), 3.14-2.94 (m, 6H), 2.75-2.56 (m, 3H), 2.34-2.24 (m, 2H), 2.17-2.05 (m, 4H), 2.00-1.65 (m, 10H), 1.48-1.44 (m, 1H), 1.42-1.23 (m, 10H), 1.20-1.14 (m, 1H), 1.14-1.01 (m, 6H), 0.97 (d, J=6.5 Hz, 3H), 0.92-0.75 (m, 9H), 0.71 (d, J=5.6 Hz, 3H) ppm.

Scheme 2. Synthetic Route for Tubulysins Modified on Mep (PA-10, 11, 12, and
16 aka PA4, PA13, and PA9, respectively)

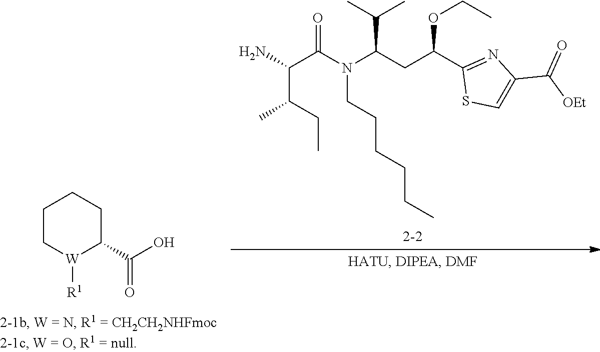

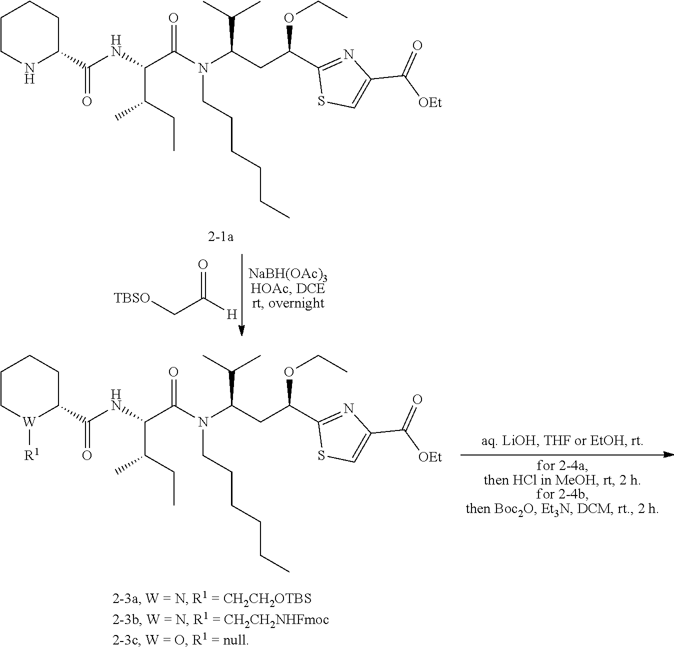

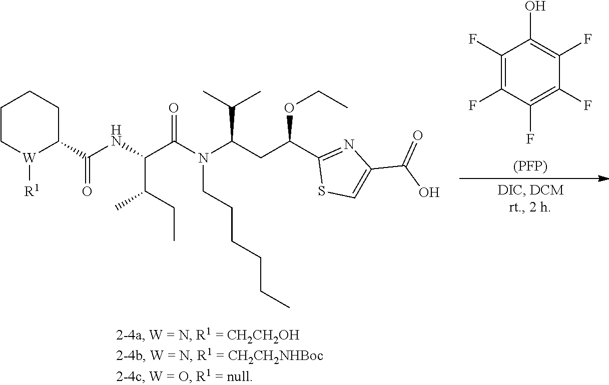

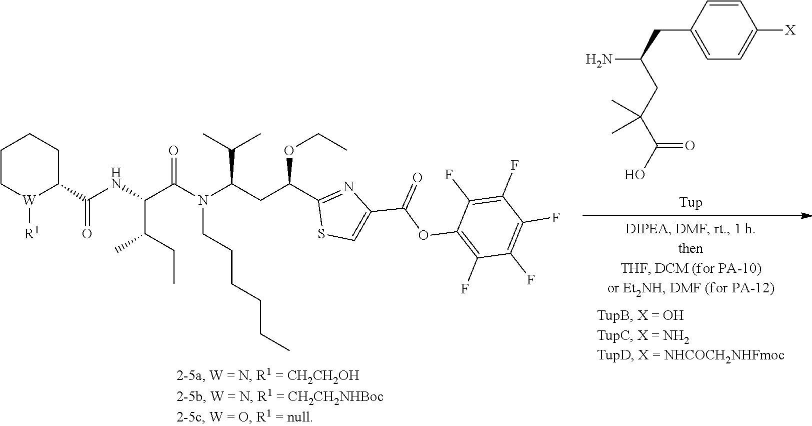

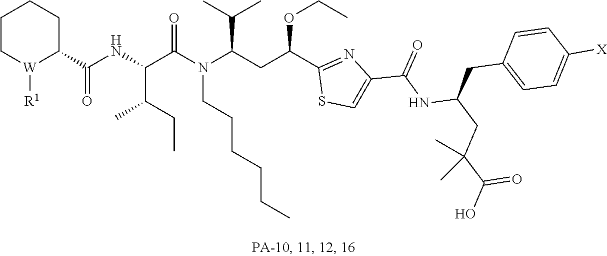

| | N/R² | W | R¹ | X | Y |
|---|---|---|---|---|---|
| PA-10 aka PA4 | 1/H | N | CH₂CH₂NH₂ | OH | H |
| PA-11 aka PA13 | 1/H | N | CH₂CH₂OH | NH₂ | H |
| PA-12 aka PA9 | 1/H | N | CH₂CH₂OH | NHCOCH₂NH₂ | H |

### Synthesis of Intermediate 2-3

### Rac-ethyl 24(1R,3R)-3-((2S,3S)-2-((R)-1-(2-((tert-butyldimethylsilyl)oxy)ethyl)piperidine-2-carboxamido)-N-hexyl-3-methylpentanamido)-1-ethoxy-4-methylpentyl)thiazole-4-carboxylate (2-3a)

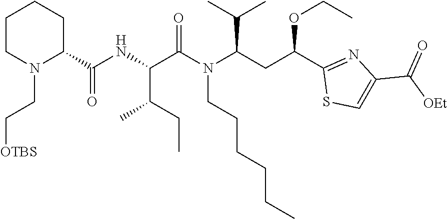 [SMILES: CCCCCCN(C(=O)[C@@H](NC(=O)[C@H]1CCCCN1CCO[Si](C)(C)C(C)(C)C)[C@@H](C)CC)[C@H](C[C@@H](OCC)c1nc(C(=O)OCC)cs1)C(C)C]

Following Alternative General Procedure I from compound 2-la (48 mg, 0.08 mmol), compound 2-3a (56 mg, 90% yield) was obtained as a white solid. ESI m/z 766.5 (M+H)⁺.

### Ethyl 24(1R,3R)-3-((2S,3S)-2-((R)-1-(2-((((9H-fluoren-9-yl)methoxy)carbonyl)amino)ethyl) piperidine-2-carboxamido)-N-hexyl-3-methylpentanamido)-1-ethoxy-4-methylpentyl) thiazole-4-carboxylate (2-3b)

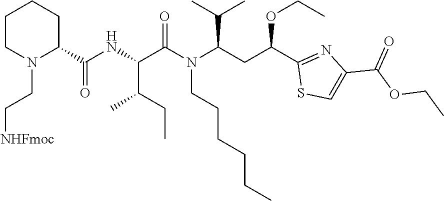

Following Alternative General Procedure IIb from compound 2-1b (50 mg, 0.10 mmol), compound 2-3b (70 mg, 80% yield) was obtained as a yellow oil. ESI m/z 873.5 (M+H)⁺.

### Ethyl 24(1R,3R)-1-ethoxy-3-((2S,3S)—N-hexyl-3-methyl-2-((R)-tetrahydro-2H-pyran-2-carboxamido)pentanamido)-4-methylpentyl)thiazole-4-carboxylate (2-3c)

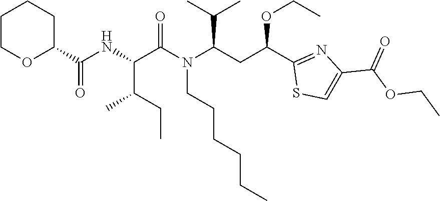 [SMILES: CCCCCCN(C(=O)[C@@H](NC(=O)[C@H]1CCCCO1)[C@@H](C)CC)[C@H](C[C@@H](OCC)c1nc(C(=O)OCC)cs1)C(C)C]

Following Alternative General Procedure IIb from compound 2-1c (47 mg, 0.12 mmol), compound 2-3c (0.10 g, 82% yield) was obtained as a white solid. ESI m/z 609.38 (M+H)⁺.

### Synthesis of Intermediate 2-4 Rac-2-((1R,3R)-1-ethoxy-3-((2S,3S)—N-hexyl-2-((R)-1-(2-hydroxyethyl)piperidine-2-carboxamido)-3-methylpentanamido)-4-methylpentyl)thiazole-4-carboxylic acid (2-4a)

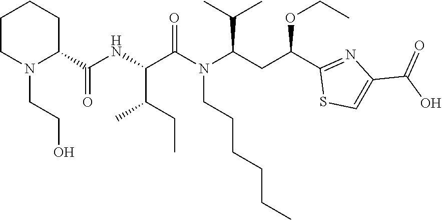 [SMILES: CCCCCCN(C(=O)[C@@H](NC(=O)[C@H]1CCCCN1CCO)[C@@H](C)CC)[C@H](C[C@@H](OCC)c1nc(C(=O)O)cs1)C(C)C]

Following Alternative General Procedure III from 2-3a (56 mg, 73 μmol), the hydrolysis product (50 mg, ESI m/z 739.5 (M+H)⁺) was obtained as a white solid, which was dissolved in a solution of HCl in methanol (4 N, 1 mL). The mixture was stirred at room temperature for two hours and monitored by LCMS. The resulting mixture was concentrated in vacuo and the residue was purified by reversed phase flash chromatography (0-100% acetonitrile in aq. ammonium bicarbonate (0.01%)) to give compound 2-4b (40 mg, 88% yield) as a yellow oil. ESI m/z: 625.5 (M+H)⁺.

### Rac-2-((1R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylic acid (2-4b)

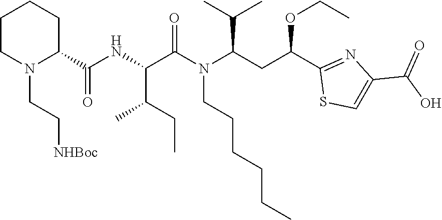 [SMILES: CCCCCCN(C(=O)[C@@H](NC(=O)[C@H]1CCCCN1CCNC(=O)OC(C)(C)C)[C@@H](C)CC)[C@H](C[C@@H](OCC)c1nc(C(=O)O)cs1)C(C)C]

Following Alternative General Procedure III from 2-3b (70 mg, 80 μmol), the hydrolysis product (Fmoc was also removed) (15 mg, ESI m/z 624.3 (M+H)⁺) was obtained as a white solid, which was dissolved in DCM (5 mL). To the DCM solution was added Boc₂O (10 mg, 48 μmol) and triethylamine (5.0 mg, 50 μmol). The mixture was stirred at room temperature for two hours and monitored by LCMS. The resulting mixture was then concentrated in vacuo and the residue was purified by prep-HPLC (5-95% acetonitrile in aq. ammonium bicarbonate (10 mM)) to give 2-4b (20 mg, 34% yield) as a white solid. ESI m/z 724.3 (M+H)⁺. 2-((1R,3R)-1-ethoxy-3-((2S,3S)—N-hexyl-3-methyl-2-((R)-tetrahydro-2H-pyran-2-carboxamido)pentanamido)-4-methylpentyl)thiazole-4-carboxylic acid (A16-3)

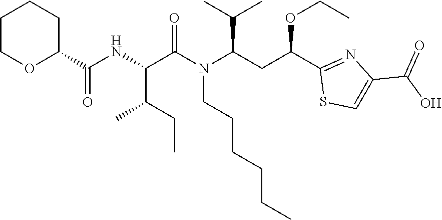 [SMILES: CCCCCCN(C(=O)[C@@H](NC(=O)[C@H]1CCCCO1)[C@@H](C)CC)[C@H](C[C@@H](OCC)c1nc(C(=O)O)cs1)C(C)C]

To a solution of 2-3c (0.10 g, 0.16 mmol) in ethanol (3 mL) was added aq. LiOH (50%, 1 mL). The reaction mixture was then stirred at room temperature for twelve hours. Then the pH was adjusted to 7.0 with aq. HCl (1 N) and the mixture was concentrated in vacuo. The crude product was purified by reversed phase flash chromatography (0-100% acetonitrile in aq. TFA (0.01%)) to give compound 2-4c (90 mg, 94% yield) as a white solid. ESI m/z 582.8 (M+H)⁺.

### Synthesis of Intermediate 2-5

### Rac-perfluorophenyl-2-((1R,3R)-1-ethoxy-3-((2S,3S)—N-hexyl-2-((R)-1-(2-hydroxyethyl)piperidine-2-carboxamido)-3-methylpentanamido)-4-methylpentyl)thiazole-4-carboxylate (2-5a)

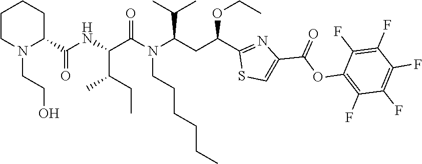 [SMILES: CCCCCCN(C(=O)[C@@H](NC(=O)[C@H]1CCCCN1CCO)[C@@H](C)CC)[C@H](C[C@@H](OCC)c1nc(C(=O)Oc2c(F)c(F)c(F)c(F)c2F)cs1)C(C)C]

Following Alternative General Procedure V from 2-4a (30 mg, 48 μmol), crude compound 2-5a (40 mg) was obtained as a white solid without purification. ESI m/z 791.3 (M+H)⁺.

### Perfluorophenyl 24(1R,3R)-3-((2S,3S)-2-((R)-1-(2-((tert-butoxycarbonyl)amino)ethyl)piperidine-2-carboxamido)-N-hexyl-3-methylpentanamido)-1-ethoxy-4-methylpentyl)thiazole-4-carboxylate (2-5b)

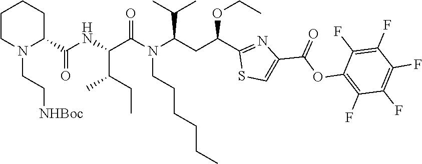 [SMILES: CCCCCCN(C(=O)[C@@H](NC(=O)[C@H]1CCCCN1CCNC(=O)OC(C)(C)C)[C@@H](C)CC)[C@H](C[C@@H](OCC)c1nc(C(=O)Oc2c(F)c(F)c(F)c(F)c2F)cs1)C(C)C]

Following Alternative General Procedure V from 2-4b (20 mg, 28 μmol), compound 2-5b (25 mg, 80% purity, 80% yield) was obtained as a yellow oil. ESI m/z 890.3 (M+H)⁺.

### 2-((1R,3R)-1-ethoxy-3-((2S,3S)—N-hexyl-3-methyl-2-((R)-tetrahydro-2H-pyran-2-carboxamido)pentanamido)-4-methylpentyl)thiazole-4-carboxylic acid (2-5c)

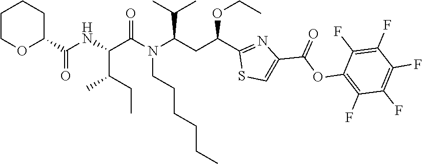 [SMILES: CCCCCCN(C(=O)[C@@H](NC(=O)[C@H]1CCCCO1)[C@@H](C)CC)[C@H](C[C@@H](OCC)c1nc(C(=O)Oc2c(F)c(F)c(F)c(F)c2F)cs1)C(C)C]

Following Alternative General Procedure V from 2-4c (15 mg, 26 μmol), compound 2-(7.7 mg, 40% yield) was obtained as a white solid. ESI m/z 748.8 (M+H)⁺.

### Synthesis of Payloads PA-10, PA-11, PA-12 (aka PA4, PA13, and PA9, Respectively)

### PA-10 aka PA4

### (S)-4-(2-((1R,3R)-3-((2S,3S)-2-((R)-1-(2-aminoethyl)piperidine-2-carboxamido)-N-hexyl-3-methylpentanamido)-1-ethoxy-4-methylpentyl)thiazole-4-carboxamido)-5-(4-hydroxyphenyl)-2,2-dimethylpentanoic acid (PA-10 aka PA4)

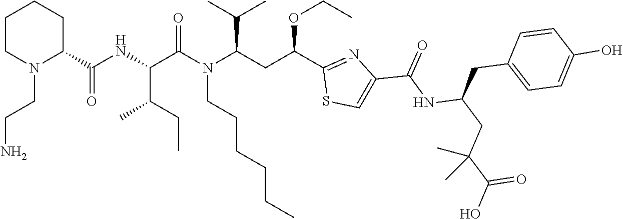 [SMILES: CCCCCCN(C(=O)[C@@H](NC(=O)[C@H]1CCCCN1CCN)[C@@H](C)CC)[C@H](C[C@@H](OCC)c1nc(C(=O)N[C@@H](Cc2ccc(O)cc2)CC(C)(C)C(=O)O)cs1)C(C)C]

Following Alternative General Procedure VI (and then Fmoc removal) from 2-5b (30 mg, 16 μmol) with TupB, payload PA-10 aka PA4 (8.8 mg, 67% yield) was obtained as a white solid. ESI m/z 942.6 (M+H)⁺. ¹H NMR (500 MHz, DMSO-d₆) δ 9.19 (br s, 1H), 8.45 (br s, 1H), 8.17-8.15 (m, 2H), 6.92 (d, J=8.2 Hz, 2H), 6.63 (d, J=8.2 Hz, 2H), 4.46 (s, 1H), 4.26 (d, J=Hz, 1H), 4.12 (s, 1H), 4.00 (s, 1H), 3.75-3.50 (m, 3H), 3.15-2.66 (m, 8H), 2.38-2.12 (m, 4H), 1.99-1.79 (m, 5H), 1.72-1.42 (m, 8H), 1.31-1.11 (m, 13H), 0.98-0.79 (m, 20H) ppm.

### PA-11 aka PA13

### (S)-5-(4-aminophenyl)-4-(2-((1R,3R)-1-ethoxy-3-((2S,3S)—N-hexyl-2-((R)-1-(2-hydroxyethyl)piperidine-2-carboxamido)-3-methylpentanamido)-4-methylpentyl) thiazole-4-carboxamido)-2,2-dimethylpentanoic acid (PA-11)

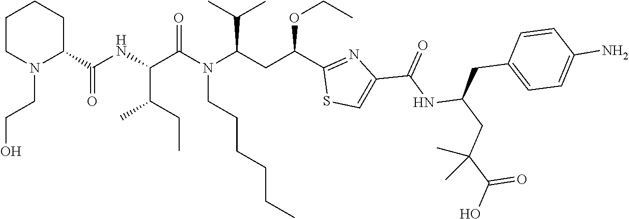 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(N)cc2)CC(C)(C)C(=O)O)cs1)C(C)C]

Following Alternative General Procedure VI from compound 2-5a (40 mg, 0.045 mmol) with TupC, payload PA-11 aka PA13 (6 mg, 15% yield) was obtained as a white solid. ESI m/z 842.5 (M+H)⁺. ¹H NMR (500 MHz, DMSO-d₆) δ 8.13 (s, 1H), 7.78 (s, 1H), 7.37 (s, 1H), 6.79 (d, J=8.2 Hz, 2H), 6.44 (d, J=8.2 Hz, 2H), 4.86 (s, 2H), 4.57-4.50 (m, 1H), 4.31 (br d, 1H), 4.19 (s, 1H), 3.80-3.71 (m, 1H), 3.52-3.40 (m, 3H), 3.12-2.89 (m, 3H), 2.81 (s, 1H), 2.70-2.54 (m, 4H), 2.33 (s, 1H), 2.19-2.10 (m, 2H), 2.05-1.90 (m, 4H), 1.79-1.65 (m, 4H), 1.53-1.51 (m, 4H), 1.35-1.25 (m, 8H), 1.20-1.15 (m, 4H), 1.05 (s, 3H), 1.03 (s, 3H), 0.98-0.77 (m, 13H), 0.70 (s, 3H) ppm.

### PA-12 aka PA9

### (S)-5-(4-(2-aminoacetamido)phenyl)-4-(2-((1R,3R)-1-ethoxy-3-((2S,3S)—N-hexyl-2-((R)-1-(2-hydroxyethyl)piperidine-2-carboxamido)-3-methylpentanamido)-4-methylpentyl) thiazole-4-carboxamido)-2,2-dimethylpentanoic acid (PA-12 aka PA9)

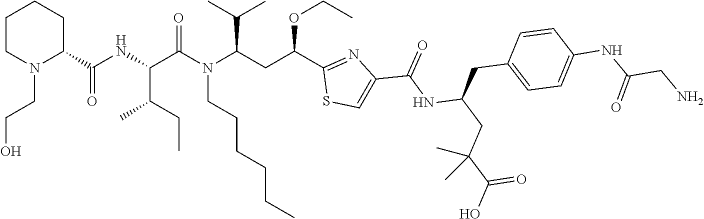 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C]

Following Alternative General Procedure VI from compound 2-5a (32 mg, 0.045 mmol) with Fmoc-TupD, payload PA-12 aka PA9 (10 mg, 30% yield) was obtained as a white solid. ESI m/z 899.5 (M+H)⁺. ¹H NMR (400 MHz, DMSO-d₆) δ 8.14 (s, 1H), 7.76 (d, J=9.9 Hz, 1H), 7.53-7.48 (m, 2H), 7.08 (d, J=7.7 Hz, 2H), 4.52 (t, J=9.5 Hz, 1H), 4.31-4.21 (m, 2H), 3.78-3.66 (m, 1H), 3.56-3.39 (m, 5H), 3.10-2.90 (m, 3H), 2.85-2.63 (m, 4H), 2.60-2.51 (m, 1H), 2.22-2.16 (m, 1H), 2.08-2.00 (m, 1H), 1.96-1.90 (m, 2H), 1.85-1.65 (m, 5H), 1.60-1.48 (m, 5H), 1.40-1.27 (m, 7H), 1.20-1.15 (m, 4H), 1.05-1.04 (m, 7H), 0.92-0.82 (m, 14H), 0.70 (s, 3H) ppm.

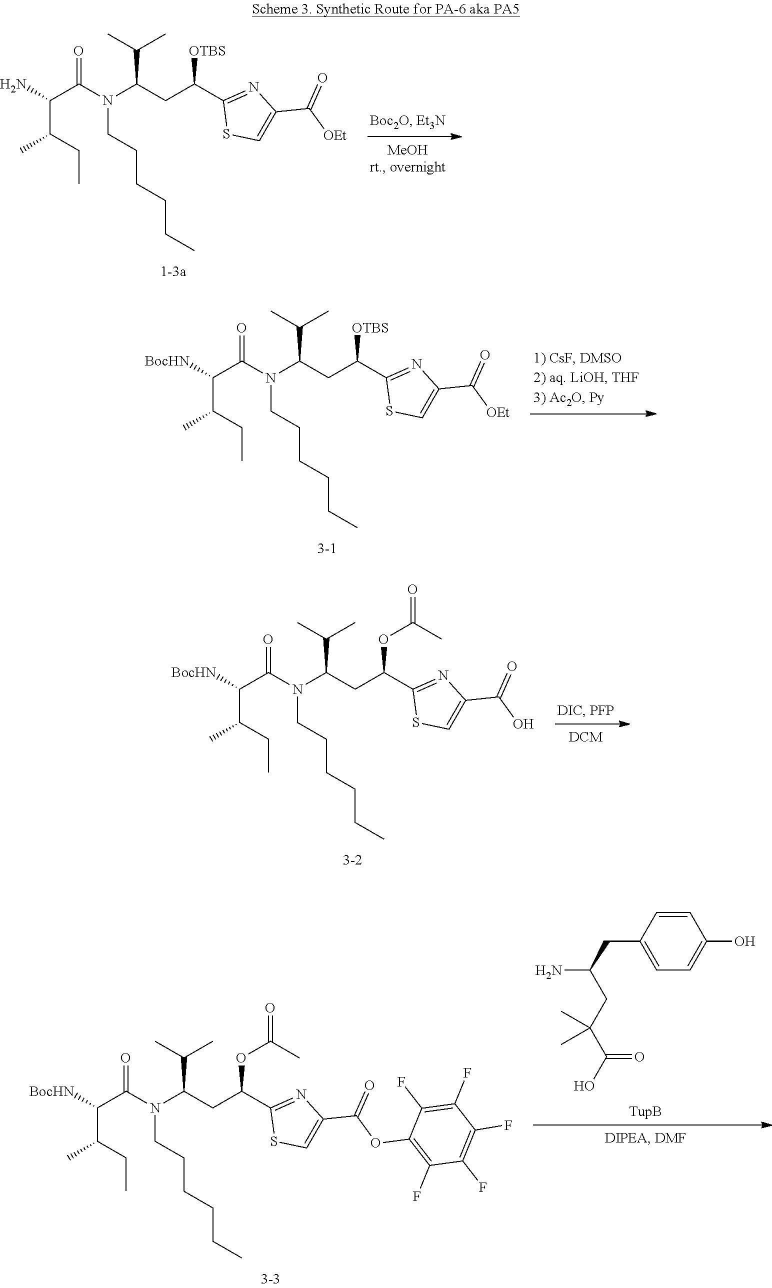 [SMILES: CCCCCCN(C(=O)[C@@H](N)[C@@H](C)CC)[C@H](C[C@@H](O[Si](C)(C)C(C)(C)C)c1nc(C(=O)OCC)cs1)C(C)C ; CCCCCCN(C(=O)[C@@H](NC(=O)OC(C)(C)C)[C@@H](C)CC)[C@H](C[C@@H](O[Si](C)(C)C(C)(C)C)c1nc(C(=O)OCC)cs1)C(C)C ; CCCCCCN(C(=O)[C@@H](NC(=O)OC(C)(C)C)[C@@H](C)CC)[C@H](C[C@@H](OC(C)=O)c1nc(C(=O)O)cs1)C(C)C ; CCCCCCN(C(=O)[C@@H](NC(=O)OC(C)(C)C)[C@@H](C)CC)[C@H](C[C@@H](OC(C)=O)c1nc(C(=O)Oc2c(F)c(F)c(F)c(F)c2F)cs1)C(C)C ; CC(C)(C[C@@H](N)Cc1ccc(O)cc1)C(=O)O]

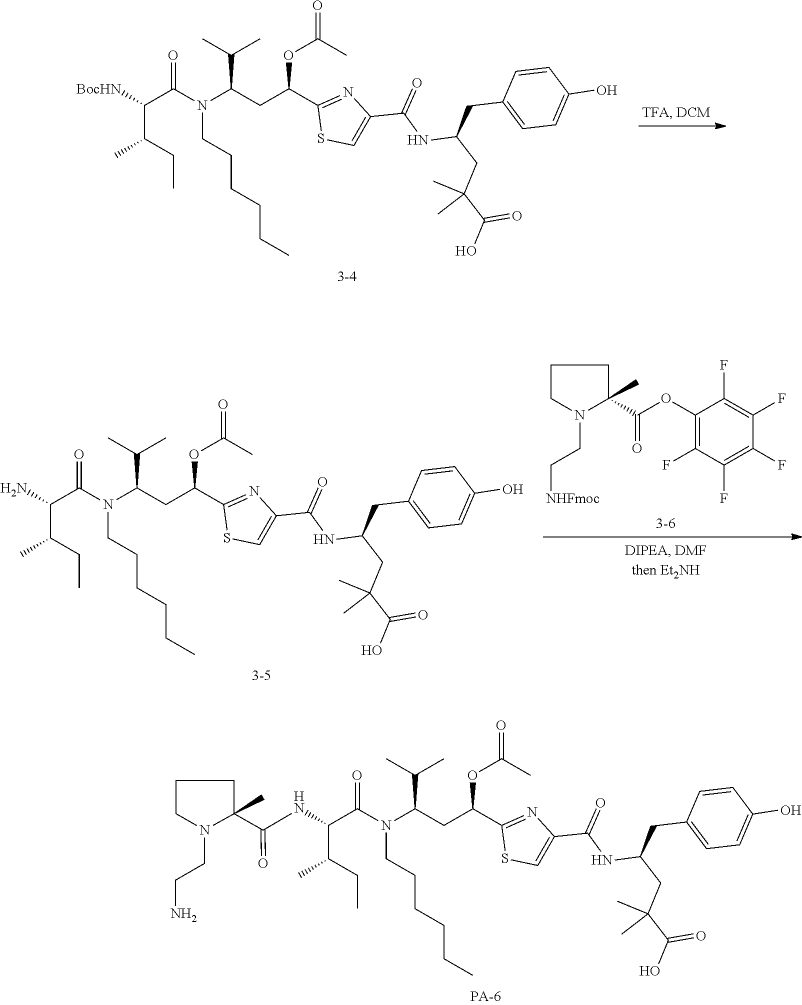

### PA-6 aka PA5

### Rac-ethyl 2-((5R,7R,10S)-10-((S)-sec-butyl)-8-hexyl-7-isopropyl-2,2,3,3,14,14-hexamethyl-9,12-dioxo-4,13-dioxa-8,11-diaza-3-silapentadecan-5-yl)thiazole-4-carboxylate (3-1)

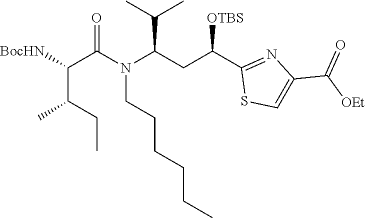 [SMILES: CCCCCCN(C(=O)[C@@H](NC(=O)OC(C)(C)C)[C@@H](C)CC)[C@H](C[C@@H](O[Si](C)(C)C(C)(C)C)c1nc(C(=O)OCC)cs1)C(C)C]

To a solution of 1-3a (90 mg, 0.15 mmol) in methanol (5 mL) was added Boc₂O (68 mg, 0.31 mmol) and triethylamine (31 mg, 0.31 mmol), and the reaction mixture was stirred at room temperature overnight. The mixture was then concentrated under reduced pressure, and the residue was purified by silica gel column chromatography (5-95% ethyl acetate in petroleumether) to give compound 3-1 (90 mg, 85% yield) as a white solid. ESI m/z 684.2 (M+H)⁺.

### (S)-4-(2-((6RS,9SR,11SR)-6-((RS)-sec-butyl)-8-hexyl-9-isopropyl-2,2-dimethyl-4,7,13-trioxo-3,12-dioxa-5,8-diazatetradecan-11-yl)thiazole-4-carboxamido)-5-(4-hydroxyphenyl)-2,2-dimethylpentanoic acid (3-4)

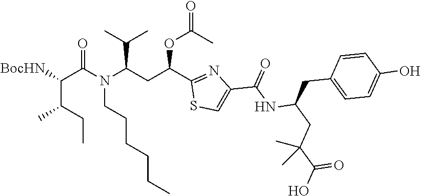 [SMILES: CCCCCCN(C(=O)[C@@H](NC(=O)OC(C)(C)C)[C@@H](C)CC)[C@H](C[C@@H](OC(C)=O)c1nc(C(=O)N[C@@H](Cc2ccc(O)cc2)CC(C)(C)C(=O)O)cs1)C(C)C]

Following Alternative General Procedures III, Iva, V, and VI successively starting from 3-1 (90 mg, 0.13 mmol) using TupB in then last step, compound 3-4 (55 mg, 50% yield) was obtained as a white solid. ESI m/z 703.4 (M-Boc+H)⁺, 803.4 (M+H)⁺.

### (S)-4-(2-((1RS,3RS)-1-acetoxy-3-((2SR,3SR)-2-amino-N-hexyl-3-methylpentanamido)-4-methylpentyl)thiazole-4-carboxamido)-5-(4-hydroxyphenyl)-2,2-dimethylpentanoic acid (3-5)

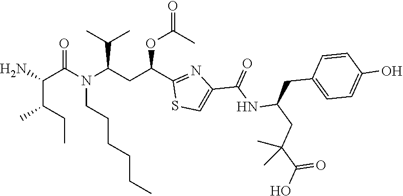 [SMILES: CCCCCCN(C(=O)[C@@H](N)[C@@H](C)CC)[C@H](C[C@@H](OC(C)=O)c1nc(C(=O)N[C@@H](Cc2ccc(O)cc2)CC(C)(C)C(=O)O)cs1)C(C)C]

To a solution of compound 3-4 (55 mg, 68 μmol) in DCM (0.6 mL) was added TFA (0.2 mL) and the reaction mixture was stirred at room temperature for three hours, which was monitored by LCMS. The resulting mixture was concentrated in vacuo. The residue was purified by prep-HPLC (5-30% acetonitrile in aq. ammonium bicarbonate (0.8 g/L)) to give compound 3-(28 mg, 60% yield) as a white solid. ESI m/z 703.3 (M+H)⁺.

### Perfluorophenyl (R)-1-(2-((((9H-fluoren-9-yl)methoxy)carbonyl)amino)ethyl)-2-methylpyrrolidine-2-carboxylate (3-6)

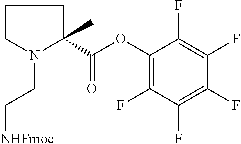

To a solution of N-Fmoc-aminoethanol (0.40 g, 1.4 mmol) in ethyl acetate (60 mL) was added IBX (1.2 g), and the reaction mixture was stirred at 80° C. for three hours, which was monitored by LCMS. After cooling to room temperature, the resulting mixture was filtered and the filter cake was washed with ethyl acetate (3×4 mL). The combined filtrate was concentrated in vacuo to give the alderhyde (0.40 g). Following Alternative General Procedure I except using the aldehyde obtained above (92 mg) instead of (tert-butyldimethylsilyloxy)acetaldehyde, compound 3-6-acid (69 mg, ESI m/z 395.2 (M+H)⁺) was obtained as a white solid. To a solution of compound 3-6-acid (20 mg, 50 μmol) in DCM (4 mL) was added PFP (19 mg, 0.10 mmol) and DIC (13 mg, 0.10 mmol), and the reaction mixture was stirred at room temperature for two hours, which was monitored by LCMS. The resulting solution was concentrated in vacuo to give compound 3-6 (crude) as a colorless oil. ESI m/z 561.2 (M+H)⁺.

### (S)-4-(2-((1RS,3RS)-1-acetoxy-3-((2SR,3SR)-2-((R)-1-(2-aminoethyl)-2-methylpyrrolidine-2-carboxamido)-N-hexyl-3-methylpentanamido)-4-methylpentyl)thiazole-4-carboxamido)-5-(4-hydroxyphenyl)-2,2-dimethylpentanoic acid (PA-6 aka PA5)

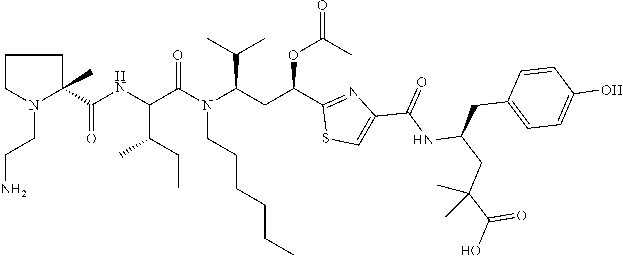 [SMILES: CCCCCCN(C(=O)C(NC(=O)[C@@]1(C)CCCN1CCN)[C@@H](C)CC)[C@H](C[C@@H](OC(C)=O)c1nc(C(=O)N[C@@H](Cc2ccc(O)cc2)CC(C)(C)C(=O)O)cs1)C(C)C]

To a mixture of compound 3-5 (28 mg, 40 μmol) in DMF (4 mL) was added compound 3-6 (23 mg, 40 μmol) and DIPEA (16 mg, 0.12 mmol), and the mixture was stirred at room temperature for an hour. The resulting mixture was purified by reversed phase flash chromatography (0-100% acetonitrile in TFA (0.01%)) to give compound Fmoc-PA-6 (24 mg) as a white solid, which was dissolved in DMF (5 mL). To the solution was added diethylamine (1 mL), and the reaction mixture was stirred at room temperature for an hour, which was monitored by LCMS. The resulting mixture was purified by prep-HPLC (5-95% acetonitrile in aq. TFA (0.01%)) to give payload PA-6 aka PA5 (15 mg, 75% yield) as a white solid. ESI m/z 857.2 (M+H)⁺. ¹H NMR (400 MHz, DMSO-d₆) δ 8.23-8.18 (m, 1H), 7.86-7.77 (m, 1H), 6.94 (d, J=8.3 Hz, 2H), 6.62 (d, J=8.1 Hz, 2H), 5.71-5.60 (m, 1H), 4.42 (t, J=9.6 Hz, 1H), 4.11-4.00 (m, 1H), 3.75-3.62 (m, 1H), 3.18-3.14 (m, 2H), 3.03-2.95 (m, 2H), 2.78-2.72 (m, 1H), 2.69-2.57 (m, 2H), 2.35-2.26 (m, 3H), 2.12 (s, 3H), 2.03-1.97 (m, 1H), 1.91-1.80 (m, 3H), 1.60-1.43 (m, 6H), 1.34-1.20 (m, 10H), 1.12-1.03 (m, 4H), 1.01-0.89 (m, 9H), 0.89-0.76 (m, 12H), 0.74-0.61 (m, 3H) ppm.

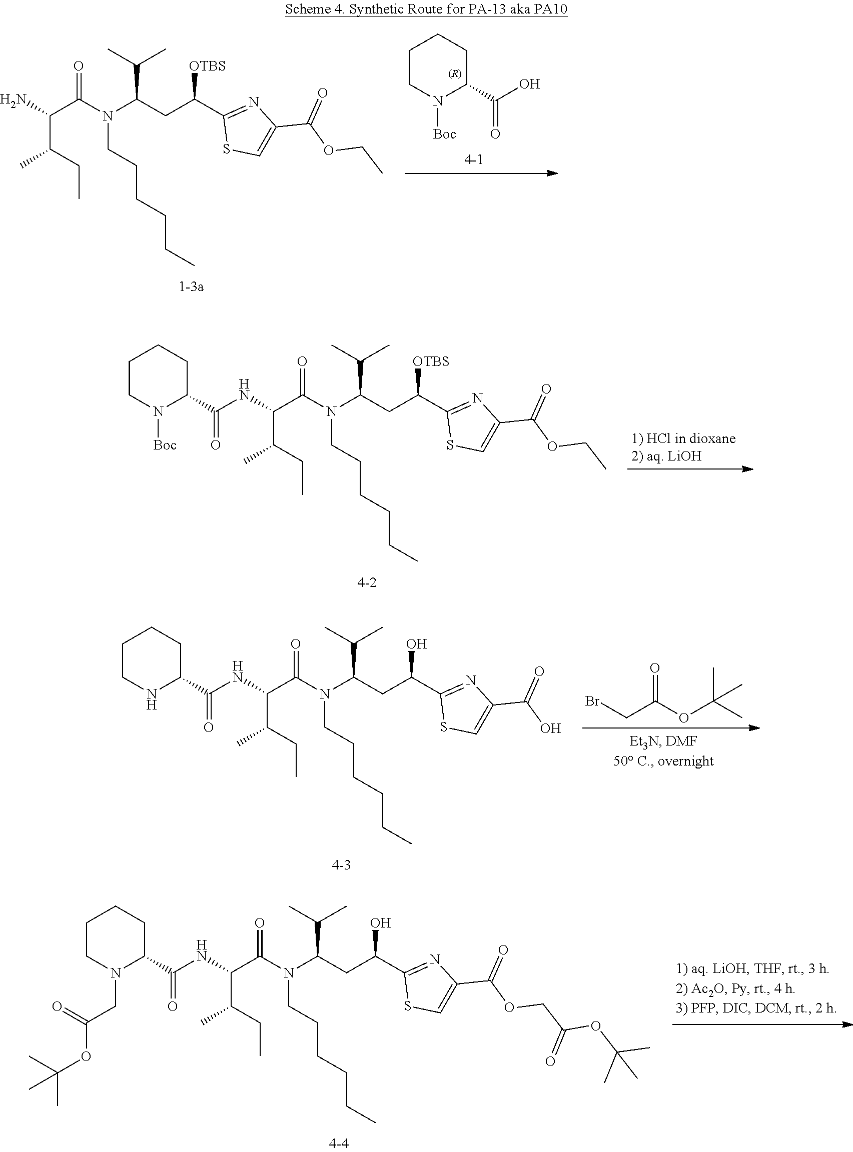

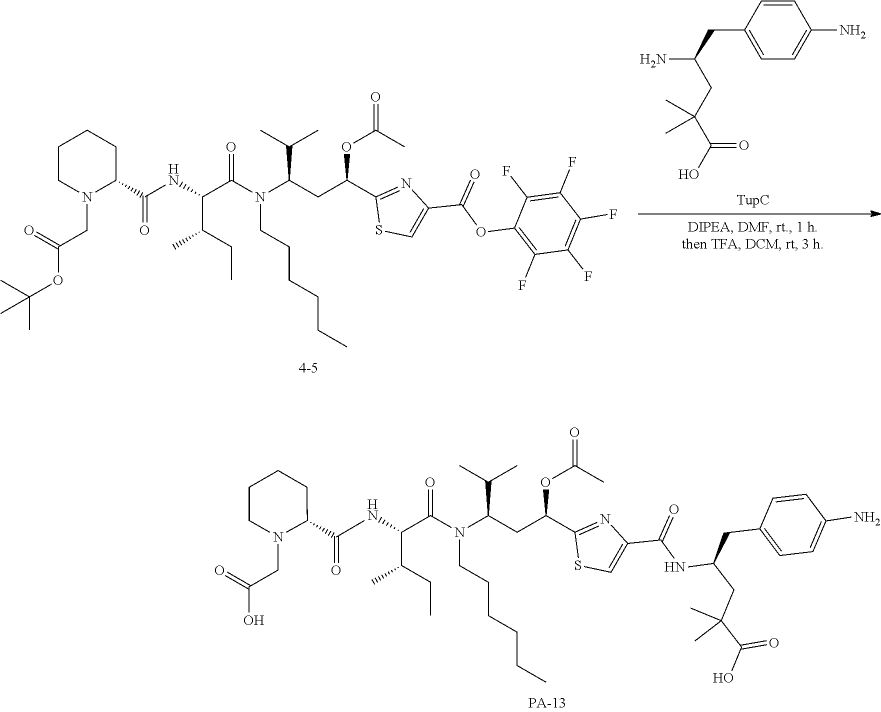 [SMILES: CCCCCCN(C(=O)[C@@H](NC(=O)[C@H]1CCCCN1CC(=O)OC(C)(C)C)[C@@H](C)CC)[C@H](C[C@@H](OC(C)=O)c1nc(C(=O)Oc2c(F)c(F)c(F)c(F)c2F)cs1)C(C)C ; CC(C)(C[C@@H](N)Cc1ccc(N)cc1)C(=O)O ; CCCCCCN(C(=O)[C@@H](NC(=O)[C@H]1CCCCN1CC(=O)O)[C@@H](C)CC)[C@H](C[C@@H](OC(C)=O)c1nc(C(=O)N[C@@H](Cc2ccc(N)cc2)CC(C)(C)C(=O)O)cs1)C(C)C]

### PA-13 aka PA10

### Ethyl 24(3S,6R,8R)-1-((R)-1-(tert-butoxycarbonyl)piperidin-2-yl)-3-((S)-sec-butyl)-5-hexyl-6-isopropyl-10,10,11,11-tetramethyl-1,4-dioxo-9-oxa-2,5-diaza-10-siladodecan-8-yl)thiazole-4-carboxylate (4-2)

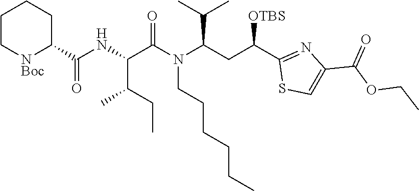 [SMILES: CCCCCCN(C(=O)[C@@H](NC(=O)[C@H]1CCCCN1C(=O)OC(C)(C)C)[C@@H](C)CC)[C@H](C[C@@H](O[Si](C)(C)C(C)(C)C)c1nc(C(=O)OCC)cs1)C(C)C]

Following Alternative General Procedure IIb starting from compound 1-3a (0.22 g, mmol) with acid 4-1, compound 4-2 (0.22 g, 73% yield) was obtained as a white solid. ESI m/z 795.5 (M+H)⁺.

### 2-((1R,3R)-3-((2S,3S)—N-hexyl-3-methyl-2-((R)-piperidine-2-carboxamido)pentanamido)-1-hydroxy-4-methylpentyl)thiazole-4-carboxylic acid (4-3)

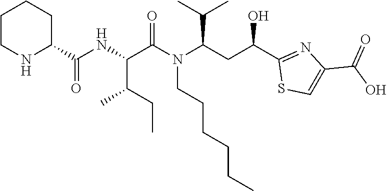 [SMILES: CCCCCCN(C(=O)[C@@H](NC(=O)[C@H]1CCCCN1)[C@@H](C)CC)[C@H](C[C@@H](O)c1nc(C(=O)O)cs1)C(C)C]

A solution of compound 4-2 (0.22 g, 0.28 mmol) in a solution of HCl in dioxane (4 N, mL) was stirred at room temperature for three hours, which was monitored by LCMS. The resulting mixture was concentrated in vacuo and the residue was dissolved in THF (3 mL). To the solution was added aq. lithium hydroxide (1 M, 0.52 mL). The resulting mixture was stirred at room temperature for three hours. The mixture was concentrated and separated by reversed phase flash chromatography (10-40% acetonitrile in aq. ammonium bicarbonate (10 mM)) to give compound 4-3 (0.13 g, 85% yield) as a white solid. ESI m/z 553.3 (M+H)⁺.

### 2-(tert-butoxy)-2-oxoethyl 2-((1R,3R)-3-((2S,3S)-2-((R)-1-(2-(tert-butoxy)-2-oxoethyl)piperidine-2-carboxamido)-N-hexyl-3-methylpentanamido)-1-hydroxy-4-methylpentyl)thiazole-4-carboxylate (4-4)

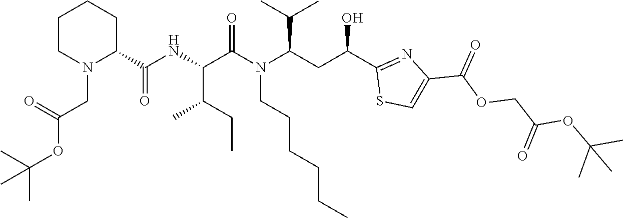 [SMILES: CCCCCCN(C(=O)[C@@H](NC(=O)[C@H]1CCCCN1CC(=O)OC(C)(C)C)[C@@H](C)CC)[C@H](C[C@@H](O)c1nc(C(=O)OCC(=O)OC(C)(C)C)cs1)C(C)C]

To a solution of compound 4-3 (0.11 g, 0.20 mmol) in DMF (5 mL) were added DIPEA (78 mg, 0.60 mmol) and tert-butyl 2-bromoacetate (0.12 g, 0.60 mmol), and the mixture was stirred at 50° C. overnight. The resulting mixture was directly purified by reversed phase flash chromatography (0-70% acetonitrile in aq. ammonium bicarbonate (10 mM)) to give compound 4-4 (0.15 g, 97% yield) as a white solid. ESI m/z 781.5 (M+H)⁺.

### Perfluorophenyl 2-((1R,3R)-1-acetoxy-3-((2S,3S)-2-((R)-1-(2-(tert-butoxy)-2-oxoethyl)piperidine-2-carboxamido)-N-hexyl-3-methylpentanamido)-4-methylpentyl)thiazole-4-carboxylate (4-5)

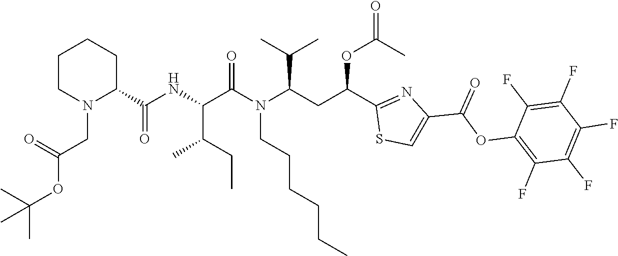 [SMILES: CCCCCCN(C(=O)[C@@H](NC(=O)[C@H]1CCCCN1CC(=O)OC(C)(C)C)[C@@H](C)CC)[C@H](C[C@@H](OC(C)=O)c1nc(C(=O)Oc2c(F)c(F)c(F)c(F)c2F)cs1)C(C)C]

Successively following Alternative General Procedures III, Iva, and V starting from compound 4-4 (0.15 g, 0.19 mmol), compound 4-5 (80 mg, 59% yield) was obtained as a white solid. ESI m/z 875.4 (M+H)⁺.

### (S)-4-(2-((1R,3R)-1-acetoxy-3-((2S,3S)-2-((R)-1-(carboxymethyl)piperidine-2-carboxamido)-N-hexyl-3-methylpentanamido)-4-methylpentyl)thiazole-4-carboxamido)-5-(4-aminophenyl)-2,2-dimethylpentanoic acid (PA-13 aka PA10)

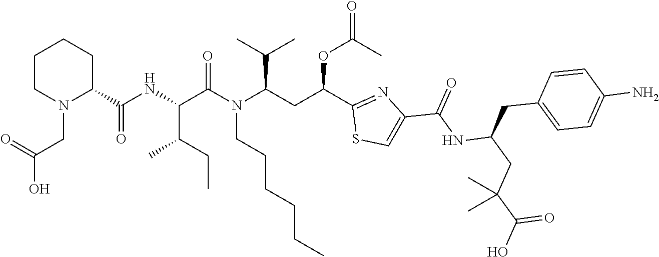 [SMILES: CCCCCCN(C(=O)[C@@H](NC(=O)[C@H]1CCCCN1CC(=O)O)[C@@H](C)CC)[C@H](C[C@@H](OC(C)=O)c1nc(C(=O)N[C@@H](Cc2ccc(N)cc2)CC(C)(C)C(=O)O)cs1)C(C)C]

Following Alternative General Procedure VI starting from compound 4-5 (80 mg, 92 μmol) with TupC, tert-butyl ester of PA-13 (45 mg) was obtained as a white solid, which was dissolved in DCM (3 mL). To the resulting solution was added TFA (1 mL) dropwise and the reaction mixture was stirred at room temperature for three hours, which was monitored by LCMS. The volatiles were removed in vacuo and the residue was purified by prep-HPLC (5-95% acetonitrile in aq. ammonium bicarbonate (10 mM)) to give PA-13 aka PA10 (11 mg, 14% yield) as a white solid. ESI m/z 871.3 (M+H)⁺. ¹H NMR (400 MHz, DMSO-d₆) δ 8.16 (s, 1H), 7.91 (d, J=8.4 Hz, 1H), 7.50 (d, J=9.2 Hz, 1H), 6.80 (d, J=4.4 Hz, 2H), 6.44 (d, J=8.4 Hz, 2H), 5.68-5.63 (m, 1H), 4.47 (t, J=9.2 Hz, 1H), 4.23-4.17 (m, 1H), 3.80-3.70 (m, 2H), 3.20-3.06 (m, 4H), 3.02-2.86 (m, 4H), 2.64-2.58 (m, 1H), 2.57-2.52 (m, 1H), 2.35-2.20 (m, 3H), 2.14 (s, 3H), 1.87-1.78 (m, 3H), 1.69-1.63 (m, 2H), 1.60-1.50 (m, 3H), 1.48-1.41 (m, 2H), 1.32-1.24 (m, 6H), 1.07-1.02 (m, 7H), 0.96 (d, J=6.4 Hz, 3H), 0.89-0.76 (m, 11H), 0.70-0.65 (m, 3H) ppm.

Scheme 5. Synthetic Route for Tubulysins Modified on Tup (PB-2, 3, 4, 5, 6, 7, 8,
and 9 aka PA14, PA15, PA16, PA30, PA17, PA18, PA19, and PA20, respectively)

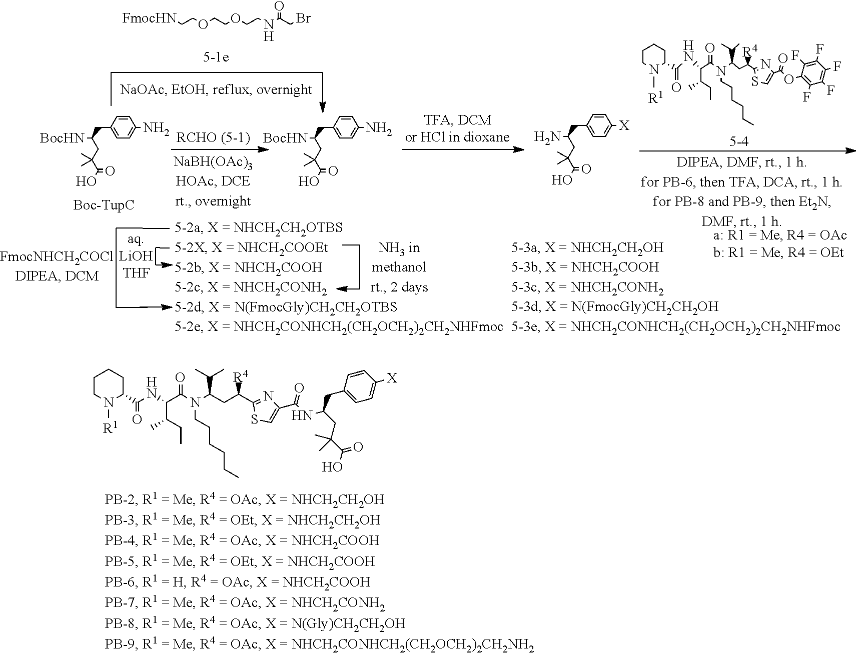

| | R¹ | R⁴ | X |
|---|---|---|---|
| PB-2 aka PA14 | Me | OAc | NHCH₂CH₂OH |
| PB-3 aka PA15 | Me | OEt | NHCH₂CH₂OH |
| PB-4 aka PA16 | Me | OAc | NHCH₂COOH |
| PB-5 aka PA30 | Me | OEt | NHCH₂COOH |
| PB-6 aka PA17 | H | OAc | NHCH₂COOH |
| PB-7 aka PA18 | Me | OAc | NHCH₂CONH₂ |
| PB-8 aka PA19 | Me | OAc | N(Gly)CH₂CH₂OH |
| PB-9 aka PA20 | Me | OAc | NHCH₂CONHCH₂(CH₂OCH₂)₂CH₂NH₂ |

### Synthesis of Intermediate 5-2

### Rac-(R)-4-((tert-butoxycarbonyl)amino)-5-(4-((2-((tert-butyldimethylsilyl)oxy)ethyl)amino)phenyl)-2,2-dimethylpentanoic acid (5-2a)

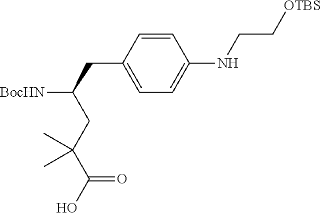 [SMILES: CC(C)(C)OC(=O)N[C@@H](Cc1ccc(NCCO[Si](C)(C)C(C)(C)C)cc1)CC(C)(C)C(=O)O]

Following Alternative General Procedure VII starting from Boc-TupC (0.34 g, 1.0 mmol) with O-TBS-acetaldehyde (0.21 g, 1.2 mmol), compound 5-2a (0.10 g, 20% yield) was obtained as a white solid. ESI m/z 495.3 (M+H)⁺.

### Rac-(R)-4-((tert-butoxycarbonyl)amino)-5-(4-((2-ethoxy-2-oxoethyl)amino)phenyl)-2,2-dimethylpentanoic acid (5-2X)

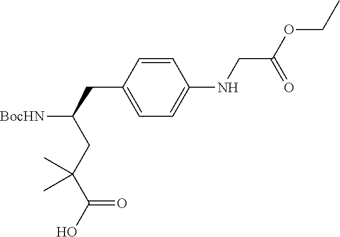 [SMILES: CCOC(=O)CNc1ccc(C[C@@H](CC(C)(C)C(=O)O)NC(=O)OC(C)(C)C)cc1]

Following Alternative General Procedure VII starting from Boc-TupC (0.34 g, 1.0 mmol) with ethyl 2-oxoacetate (0.12 g, 1.2 mmol), compound 5-2X (0.35 g, 90% yield) was obtained as a white solid. ESI m/z 367.2 (M-t-Bu+H)⁺.

### Rac-(R)-4-((tert-butoxycarbonyl)amino)-5-(4-((carboxymethyl)amino)phenyl)-2,2-dimethylpentanoic acid (5-2b)

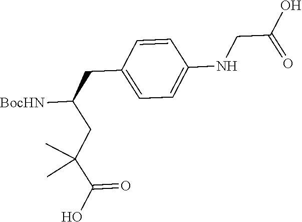 [SMILES: CC(C)(C)OC(=O)N[C@@H](Cc1ccc(NCC(=O)O)cc1)CC(C)(C)C(=O)O]

To a solution of compound 5-2X (0.38 g, 0.90 mmol) in THF (5 mL) was added aq. lithium hydroxide (2 mL, 0.92 mmol), and the mixture was stirred at room temperature for two hours, which was monitored by LCMS. The resulting mixture was acidified by aq. HCl (2 N) to pH<7 and diluted with water (10 mL). The mixture was extracted with ethyl acetate (3×20 mL). The combined organic solution was concentrated and the crude product was purified by reversed phase flash chromatography (0-100% acetonitrile in aq. TFA (0.01%)) to give compound 5-2b (0.31 g, 90% yield) as a white solid. ESI m/z 339.2 (M-t-Bu+H)⁺.

### Rac-(R)-5-(4-((2-amino-2-oxoethyl)amino)phenyl)-4-((tert-butoxycarbonyl)amino)-2,2-dimethylpentanoic acid (5-2c)

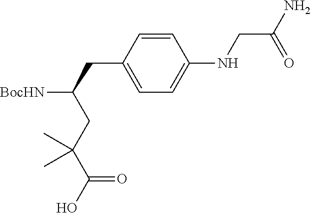 [SMILES: CC(C)(C)OC(=O)N[C@@H](Cc1ccc(NCC(N)=O)cc1)CC(C)(C)C(=O)O]

A solution of compound 5-2X (0.14 g, 0.34 mmol) in a solution of ammonia in methanol (7 M, 5 mL) was stirred at room temperature for two days. The volatiles were then removed in vacuo to give compound 5-2c (0.13 g, 99% yield), which was used in the next step without further purification. ESI m/z 416.3 (M+Na)⁺, 293.2 (M-Boc+H)⁺.

### Rac-(R)-5-(4-(2-((((9H-fluoren-9-yl)methoxy)carbonyl)amino)-N-(2-((tert-butyldimethylsilyl)oxy)ethyl)acetamido)phenyl)-4-((tert-butoxycarbonyl)amino)-2,2-dimethylpentanoic acid (5-2d)

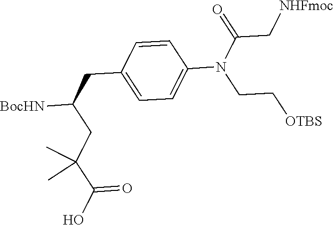

To a solution of N-Fmoc-glycine (0.45 g, 1.5 mmol) in dry DCM (10 mL) was added oxalyl chloride (0.29 g, 2.3 mmol) and DMF (1 drop), and the reaction mixture was stirred at room temperature for an hour, which was monitored by LCMS. The volatiles were then removed in vacuo and the chloride was dissolved in DMF (4 mL). To the resulting solution was added compound 5-2a (0.25 g, 0.51 mmol) and DIPEA (0.26 g, 2.0 mmol), and the reaction mixture was stirred at room temperature for an hour, which was monitored by LCMS. The resulting mixture was directly purified by reversed phase flash chromatography (0-100% acetonitrile in aq. TFA (0.01%)) to give compound 5-2d (0.11 g, 29% yield) as a white solid. ESI m/z 718.1 (M-t-Bu+H)⁺.

### Rac-(R)-5-(4-((1-(9H-fluoren-9-yl)-3,14-dioxo-2,7,10-trioxa-4,13-diazapentadecan-15-yl)amino)phenyl)-4-((tert-butoxycarbonyl)amino)-2,2-dimethylpentanoic acid (5-2e)

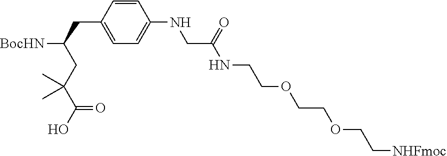

To a mixture of N-Fmoc-PEG₂-amine (CAS: 444727-01-9, 0.69 g, 1.9 mmol) in DCM was added 2-bromoacetyl bromide (0.38 g, 1.9 mmol) and DIPEA (0.72 g, 5.6 mmol), and the reaction mixture was stirred at room temperature for an hour. The resulting mixture was separated by reversed phase flash chromatography (0-100% acetonitrile in aq. TFA (0.01%)) to give bromide (0.36 g, ESI m/z 491.2 (M+H)⁺) as a white solid, which was dissolved in ethanol (25 mL). To the resulting solution was added Boc-TupC (0.31 g, 0.91 mmol) and sodium acetate (0.12 g, 1.5 mmol), and the mixture was stirred at 80° C. overnight. After cooling to room temperature, the resulting mixture was separated by reversed phase flash chromatography (0-100% acetonitrile in aq. TFA (0.01%)) to give compound 5-2e (0.23 g, 43% yield) as a white solid. ESI m/z 747.4 (M+H)⁺.

### Synthesis of Intermediate 5-3

### Rac-(R)-4-amino-5-(4-((2-hydroxyethyl)amino)phenyl)-2,2-dimethylpentanoic acid (5-3a)

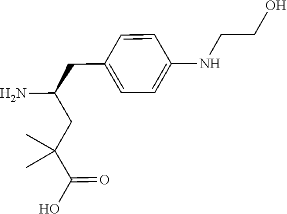 [SMILES: CC(C)(C[C@@H](N)Cc1ccc(NCCO)cc1)C(=O)O]

A solution of compound 5-2a (0.10 g, 0.20 mmol) in a solution of HCl in dioxane (4 N, 5 mL) was stirred at room temperature for twelve hours. The volatiles were removed in vacuo to give compound 5-3a (50 mg, 90% yield) as a white solid, which was used in the next step without further purification. ESI m/z 281.3 (M+H)⁺.

### Rac-(R)-4-amino-5-(4-((carboxymethyl)amino)phenyl)-2,2-dimethylpentanoic acid (5-3b)

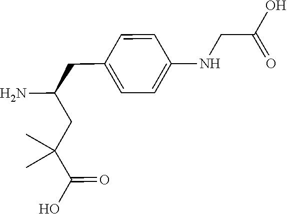 [SMILES: CC(C)(C[C@@H](N)Cc1ccc(NCC(=O)O)cc1)C(=O)O]

To a mixture of compound 5-2b (0.31 g, 0.80 mmol) in DCM (5 mL) was added TFA (1 mL), and the reaction mixture was stirred at room temperature for two hours, which was monitored by LCMS. The volatiles were removed in vacuo and the residue was purified by reversed phase flash chromatography (0-100% acetonitrile in aq. TFA (0.01%)) to give compound (0.20 g, 90% yield) as a white solid. ESI m/z 295.2 (M+H)⁺.

### Rac-(R)-4-amino-5-(4-((2-amino-2-oxoethyl)amino)phenyl)-2,2-dimethylpentanoic acid (5-3c)

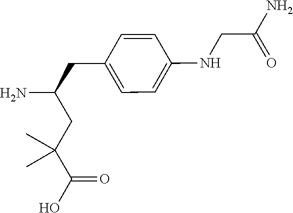 [SMILES: CC(C)(C[C@@H](N)Cc1ccc(NCC(N)=O)cc1)C(=O)O]

To a solution of compound 5-2c (0.13 g, 0.33 mmol) in DCM (5 mL) was added TFA (1 mL), and the reaction mixture was stirred at room temperature for three hours, which was monitored by LCMS. The volatiles were removed in vacuo. The residue was purified by prep-HPLC (0-30% acetonitrile in aq. TFA (0.1%)) to give compound 5-3c (98 mg, 99% yield) as a white solid. ESI m/z 294.3 (M+H)⁺.

### Rac-(R)-5-(4-(2-((((9H-fluoren-9-yl)methoxy)carbonyl)amino)-N-(2-hydroxyethyl)acetamido)phenyl)-4-amino-2,2-dimethylpentanoic acid (5-3d)

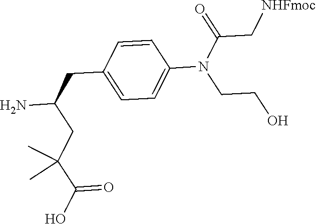

To a solution of compound 5-2d (0.11 g, 0.15 mmol) in DCM (0.6 mL) was added TFA (0.2 mL), and the reaction mixture was stirred at room temperature for three hours, which was monitored by LCMS. The volatiles were removed in vacuo and the residue was purified by prep-HPLC (0-30% acetonitrile in aq. ammonium bicarbonate (10 mM)) to give compound 5-3d (71 mg, 86% yield) as a white solid. ESI m/z 560.1 (M+H)⁺.

### Rac-(R)-5-(4-((1-(9H-fluoren-9-yl)-3,14-dioxo-2,7,10-trioxa-4,13-diazapentadecan-15-yl)amino)phenyl)-4-amino-2,2-dimethylpentanoic acid (5-3e)

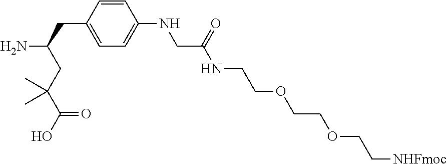

To a solution of compound 5-2e (0.23 g, 0.31 mmol) in DCM (10 mL) was added TFA (1 mL), and the reaction mixture was stirred at room temperature for three hours, which was monitored by LCMS. The volatiles were removed in vacuo and the residue was purified by prep-HPLC (0-30% acetonitrile in aq. TFA (0.01%)) to give compound 5-3e (0.20 g, 96% yield) as a white solid. ESI m/z 647.3 (M+H)⁺.

### Synthesis of Payloads Modified on Tup (PB-2, 3, 4, 5, 6, 7, 8, and 9 aka PA14, PA15, PA16, PA30, PA17, PA18, PA19, and PA20, respectively)

### PB-2 aka PA14

### Rac-(R)-4-(2-((1S,3S)-1-acetoxy-3-((2R,3R)—N-hexyl-3-methyl-2-((S)-1-methylpiperidine-2-carboxamido)pentanamido)-4-methylpentyl)thiazole-4-carboxamido)-acid (PB-2 aka PA14)

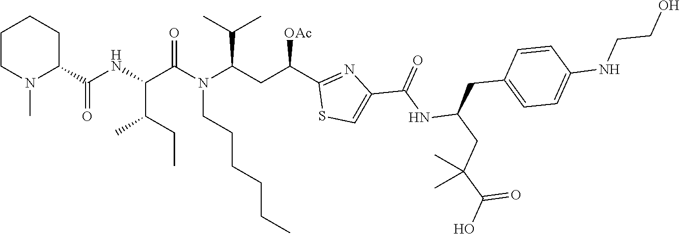 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCO)cc2)CC(C)(C)C(=O)O)cs1)C(C)C]

Following Alternative General Procedure VI starting from PFP ester 5-4a (40 mg, 51 μmol) with aniline 5-3a (14 mg, 50 μmol), payload PB-2 aka PA14 (12 mg, 25% yield) was obtained as a white solid. ESI m/z 871.5 (M+H)⁺. ¹H NMR (500 MHz, DMSO-d₆) δ 8.16 (s, 1H), 7.65 (d, J=9.4 Hz, 1H), 7.56 (br s, 1H), 6.87 (d, J=8.4 Hz, 2H), 6.46 (d, J=8.4 Hz, 2H), 5.65 (d, J=13.1 Hz, 1H), 5.28 (s, 1H), 4.64 (s, 1H), 4.48 (t, J=9.2 Hz, 1H), 4.22-4.17 (m, 1H), 3.72-3.65 (m, 1H), 3.50 (s, 3H), 3.08-2.95 (m, 3H), 2.85-2.82 (m, 1H), 2.66-2.55 (m, 3H), 2.31-2.23 (m, 2H), 2.13 (s, 3H), 2.07 (s, 3H), 1.98-1.78 (m, 5H), 1.68-1.52 (m, 6H), 1.41-1.23 (m, 9H), 1.05-1.04 (m, 6H), 0.96-0.94 (m, 3H), 0.92-0.73 (m, 9H), 0.69 (d, J=6.1 Hz, 3H) ppm.

### PB-3 aka PA15

### Rac-(R)-4-(2-((1S,3S)-1-ethoxy-3-((2R,3R)—N-hexyl-3-methyl-2-((S)-1-methylpiperidine-2-carboxamido)pentanamido)-4-methylpentyl)thiazole-4-carboxamido)-acid (PB-3 aka PA15)

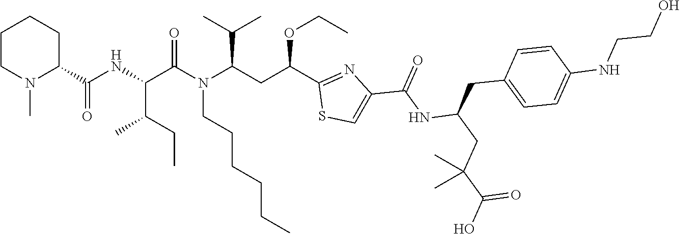 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCO)cc2)CC(C)(C)C(=O)O)cs1)C(C)C]

Following Alternative General Procedure VI starting from PFP ester 5-4b (30 mg, 39 μmol) with aniline 5-3a (12 mg, 43 μmol), payload PB-3 aka PA15 (5 mg, 15% yield) was obtained as a white solid. ESI m/z 857.3 (M+H)⁺. ¹H NMR (500 MHz, DMSO-d₆) δ 8.15 (s, 1H), 7.72 (s, 1H), 6.86 (d, J=8.3 Hz, 2H), 6.47 (d, J=8.4 Hz, 2H), 5.28 (s, 1H), 4.64 (s, 1H), 4.51 (t, J=9.5 Hz, 1H), 4.30 (d, J=12.0 Hz, 1H), 4.18 (s, 1H), 3.73 (d, J=12.3 Hz, 1H), 3.55-3.51 (m, 3H), 3.03-2.93 (m, 4H), 2.88-2.83 (m, 1H), 2.69-2.51 (m, 2H), 2.09 (s, 3H), 2.02-1.55 (m, 11H), 1.54-1.34 (m, 4H), 1.30 (s, 6H), 1.21-1.07 (m, 5H), 1.04 (s, 3H), 1.02 (s, 3H), 0.91-0.80 (m, 13H), (s, 3H) ppm.

### PB-4 aka PA16

### Rac-(R)-4-(2-((1S,3S)-1-acetoxy-3-((2R,3R)—N-hexyl-3-methyl-2-((S)-1-methylpiperidine-2-carboxamido)pentanamido)-4-methylpentyl)thiazole-4-carboxamido)-acid (PB-4 aka PA16)

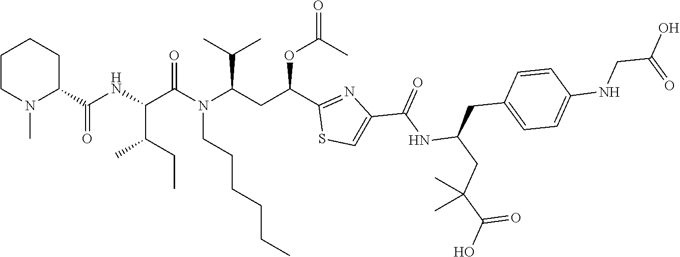 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C]

Following Alternative General Procedure VI starting from PFP ester 5-4a (30 mg, 39 μmol) with aniline 5-3b (12 mg, 41 μmol), payload PB-4 aka PA16 (5 mg, 14% yield) was obtained as a white solid. ESI m/z 885.2 (M+H)⁺. ¹H NMR (500 MHz, DMSO-d₆) δ 8.16 (s, 1H), 7.67 (d, J=9.2 Hz, 1H), 7.56 (d, J=8.7 Hz, 1H), 6.88 (d, J=8.4 Hz, 2H), 6.43 (d, J=8.4 Hz, 2H), 5.65 (d, J=13.2 Hz, 1H), 4.49 (t, J=9.2 Hz, 1H), 4.21 (s, 1H), 3.63 (s, 2H), 3.03-2.93 (m, 2H), 2.84-2.80 (m, 1H), 2.68-2.57 (m, 3H), 2.33-2.26 (m, 2H), 2.13 (s, 3H), 2.08 (s, 3H), 1.97-1.81 (m, 4H), 1.69-1.46 (m, 7H), 1.41-1.24 (m, 8H), 1.21-1.11 (m, 3H), 1.06 (s, 3H), 1.04 (s, 3H), (d, J=6.4 Hz, 3H), 0.89-0.78 (m, 9H), 0.69 (d, J=6.1 Hz, 3H) ppm.

### PB-5 aka PA30

### Rac-(R)-5-(4-((carboxymethyl)amino)phenyl)-4-(2-((1S,3S)-1-ethoxy-3-((2R,3R)—N-hexyl-3-methyl-2-((S)-1-methylpiperidine-2-carboxamido)pentanamido)-4-methylpentyl)thiazole-4-carboxamido)-2,2-dimethylpentanoic acid (PB-5 aka PA30)

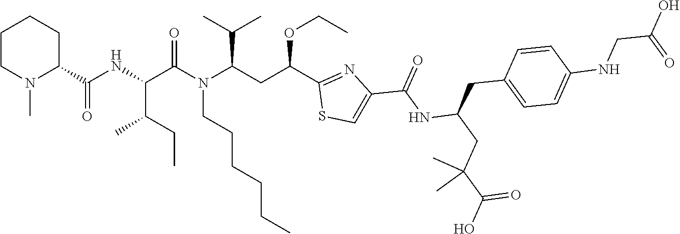 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCC(=O)O)cc2)CC(C)(C)C(=O)O)cs1)C(C)C]

Following Alternative General Procedure VI starting from PFP ester 5-4b (65 mg, 85 μmol) with aniline 5-3b (25 mg, 85 μmol), payload PB-5 aka PA30 (18 mg, 21% yield) was obtained as a white solid. ESI m/z 871.4 (M+H)⁺. ¹H NMR (400 MHz, DMSO-d₆) δ 8.16 (s, 1H), 7.42 (s, 2H), 6.87 (d, J=8.3 Hz, 2H), 6.44 (d, J=8.5 Hz, 2H), 4.56-4.47 (m, 2H), 4.34-4.26 (m, 2H), 4.25-4.15 (m, 2H), 3.72 (s, 4H), 3.01-2.92 (m, 3H), 2.69-2.65 (m, 2H), 2.35-2.30 (m, 2H), 2.24-2.15 (m, 3H), 1.99-1.89 (m, 4H), 1.70-1.55 (m, 6H), 1.52-1.41 (m, 4H), 1.35-1.27 (m, 6H), 1.17 (t, J=6.8 Hz, 4H), 1.04 (d, J=7.8 Hz, 6H), 0.93-0.79 (m, 12H), 0.70 (s, 3H) ppm.

### PB-6 aka PA17

### Rac-(R)-4-(2-((1S,3S)-1-acetoxy-3-((2R,3R)—N-hexyl-3-methyl-2-((S)-piperidine-2-carboxamido)pentanamido)-4-methylpentyl)thiazole-4-carboxamido)-5-(4-((carboxymethyl)amino)phenyl)-2,2-dimethylpentanoic acid (PB-6 aka PA17)

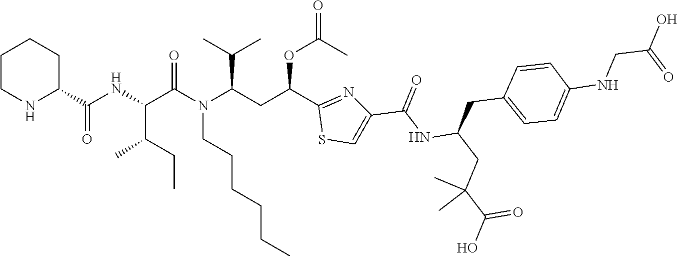 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C]

Following Alternative General Procedure V starting from acid 1-7 (70 mg, 0.10 mmol), corresponding crude PFP ester (0.12 g, ESI m/z 883.4 (M+Na)⁺) was obtained as a yellow oil. Following Alternative General Procedure VI (and then Boc removal) using the PFP ester with aniline 5-3b (81 mg, 0.20 mmol), payload PB-6 aka PA17 (6 mg, 7% yield from 1-7) was obtained as a white solid. ESI m/z 871.3 (M+H)⁺. ¹H NMR (400 MHz, DMSO-d₆) δ 8.36 (d, J=9.6 Hz, 1H), 8.19 (s, 1H), 7.19 (m, 1H), 6.91 (d, J=8.4 Hz, 2H), 6.45 (d, J=8.4 Hz, 2H), 5.81-5.77 (m, 1H), 4.44-4.26 (m, 3H), 3.21-3.12 (m, 2H), 2.88-2.70 (m, 4H), 2.64-2.60 (m, 2H), 2.30-2.24 (m, 2H), 2.20 (s, 3H), 2.11-2.05 (m, 1H), 1.94-1.84 (m, 3H), 1.76-1.55 (m, 7H), 1.47-1.36 (m, 4H), 1.27-1.21 (m, 2H), 1.18-1.07 (m, 2H), 1.09-1.01 (m, 11H), 0.99-0.95 (m, 1H), 0.87-0.85 (m, 4H), (m, 6H), 0.74 (d, J=6.8 Hz, 3H) ppm.

### PB-7 aka PA18

### Rac-(R)-4-(2-((1S,3S)-1-acetoxy-3-((2R,3R)—N-hexyl-3-methyl-2-((S)-1-methylpiperidine-2-carboxamido)pentanamido)-4-methylpentyl)thiazole-4-carboxamido)-acid (PB-7 aka PA18)

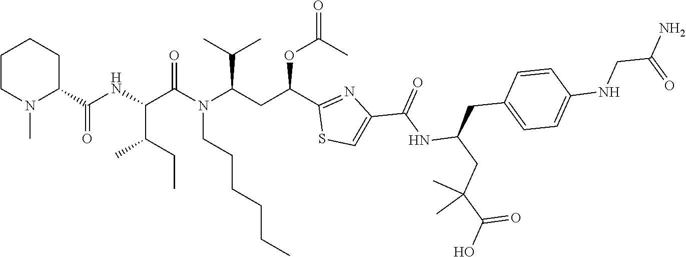 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(N)=O)cc2)CC(C)(C)C(=O)O)cs1)C(C)C]

Following Alternative General Procedure VI starting from PFP ester 5-4a (0.13 g, 0.16 mmol) with aniline 5-3c (98 mg, 0.32 mmol), payload PB-7 aka PA18 (3.6 mg, 2% yield) was obtained as a white solid. ESI m/z 442.9 (M/2+H)⁺. ¹H NMR (400 MHz, DMSO-d₆) δ 9.74-9.68 (m, 1H), 9.13 (d, J=9.8 Hz, 1H), 8.19 (s, 1H), 7.33-7.29 (m, 1H), 7.24-7.19 (m, 1H), 7.10-7.07 (m, 1H), 6.90 (d, J=8.4 Hz, 2H), 6.70-6.65 (m, 1H), 6.44 (d, J=8.3 Hz, 2H), 5.67-5.62 (m, 1H), (m, 1H), 4.54-4.50 (m, 1H), 4.24-4.19 (m, 1H), 3.79-3.75 (m, 1H), 3.10-3.05 (m, 2H), 2.68-2.61 (m, 4H), 2.14 (s, 3H), 2.03-1.95 (m, 5H), 1.83-1.74 (m, 4H), 1.49-1.40 (m, 4H), 1.28-1.22 (m, 13H), 1.06 (s, 3H), 1.05 (s, 3H), 0.97 (d, J=6.5 Hz, 3H), 0.87-0.79 (m, 10H), 0.74-0.67 (m, 3H) ppm.

### PB-8 aka PA19

### Rac-(R)-4-(2-((1S,3S)-1-acetoxy-3-((2R,3R)—N-hexyl-3-methyl-2-((S)-1-methylpiperidine-2-carboxamido)pentanamido)-4-methylpentyl)thiazole-4-carboxamido)-acid (PB-8 aka PA19)

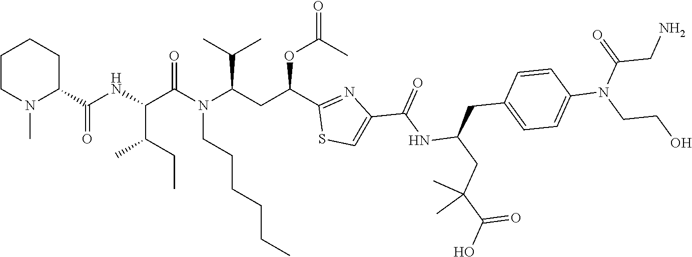 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N(CCO)C(=O)CN)cc2)CC(C)(C)C(=O)O)cs1)C(C)C]

Following Alternative General Procedure VI (and then Fmoc removal) starting from PFP ester 5-4a (44 mg, 57 μmol) with aniline 5-3d (32 mg, 57 μmol), payload PB-8 aka PA19 (20 mg, 37% yield) was obtained as a white solid. ESI m/z 1151.5 (M+H)⁺. ¹H NMR (400 MHz, DMSO-d₆) δ 12.11 (s, 1H), 9.79-9.69 (m, 1H), 9.11 (d, J=8.5 Hz, 1H), 8.14 (s, 1H), 8.05 (s, 1H), 7.96-7.91 (m, 2H), 7.88-7.82 (m, 1H), 7.29 (d, J=8.2 Hz, 2H), 7.26 (d, J=8.2 Hz, 2H), 5.64 (d, J=12.3 Hz, 1H), 4.52 (t, J=9.5 Hz, 2H), 4.34-4.19 (m, 2H), 4.16-4.05 (m, 1H), 3.83-3.65 (m, 4H), 3.28-3.22 (m, 2H), 3.17-3.03 (m, 3H), 2.88-2.77 (m, 2H), 2.68-2.60 (m, 3H), 2.35-2.26 (m, 2H), 2.16-2.05 (m, 4H), 2.03-1.90 (m, 3H), 1.82-1.53 (m, 8H), 1.42-1.28 (m, 7H), 1.17-1.12 (m, 1H), 1.10-1.01 (m, 6H), 0.97 (d, J=6.4 Hz, 3H), 0.90-0.78 (m, 9H), 0.71 (d, J=5.6 Hz, 3H) ppm.

### PB-9 aka PA20

### (RS)-4-(2-((1R,3R)-1-acetoxy-3-((2S,3S)—N-hexyl-3-methyl-2-((R)-1-methylpiperidine-2-carboxamido)pentanamido)-4-methylpentyl)thiazole-4-carboxamido)-dimethylpentanoic acid (PB-9 aka PA20)

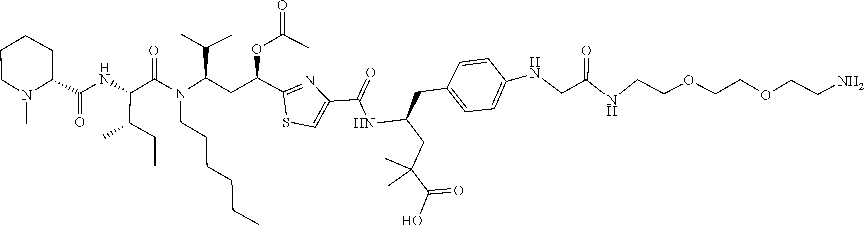 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCN)cc2)CC(C)(C)C(=O)O)cs1)C(C)C]

Following Alternative General Procedure VI (and then Fmoc removal) starting from PFP ester 5-4a (0.14 g, 0.18 mmol) with aniline 5-3e (0.12 g, 0.18 mmol), payload PB-9 aka PA20 (53 mg, 29% yield) was obtained as a white solid. ESI m/z 508.3 (M/2+H)⁺. 1H NMR (500 MHz, DMSO-d₆) δ 8.15 (d, J=10.3 Hz, 1H), 7.73 (s, 1H), 7.62 (s, 1H), 7.52 (d, J=8.9 Hz, 1H), 6.81 (d, J=8.3 Hz, 2H), 6.44 (d, J=8.3 Hz, 2H), 5.61 (d, J=12.6 Hz, 1H), 4.49 (t, J=9.3 Hz, 1H), 4.12 (s, 1H), 3.68 (s, 1H), 3.61-3.56 (m, 1H), 3.00 (s, 1H), 2.84 (d, J=11.2 Hz, 1H), 2.68-2.59 (m, 1H), 2.41-2.30 (m, 2H), 2.10-2.08 (m, 6H), 1.98-1.84 (m, 5H), 1.75-1.47 (m, 7H), 1.47-1.40 (m, 3H), 1.40-1.21 (m, 7H), 1.23-1.06 (m, 3H), 1.03 (s, 6H), 0.97 (d, J=6.4 Hz, 3H), 0.85-0.82 (m, 9H), 0.68 (d, J=5.8 Hz, 3H) ppm.

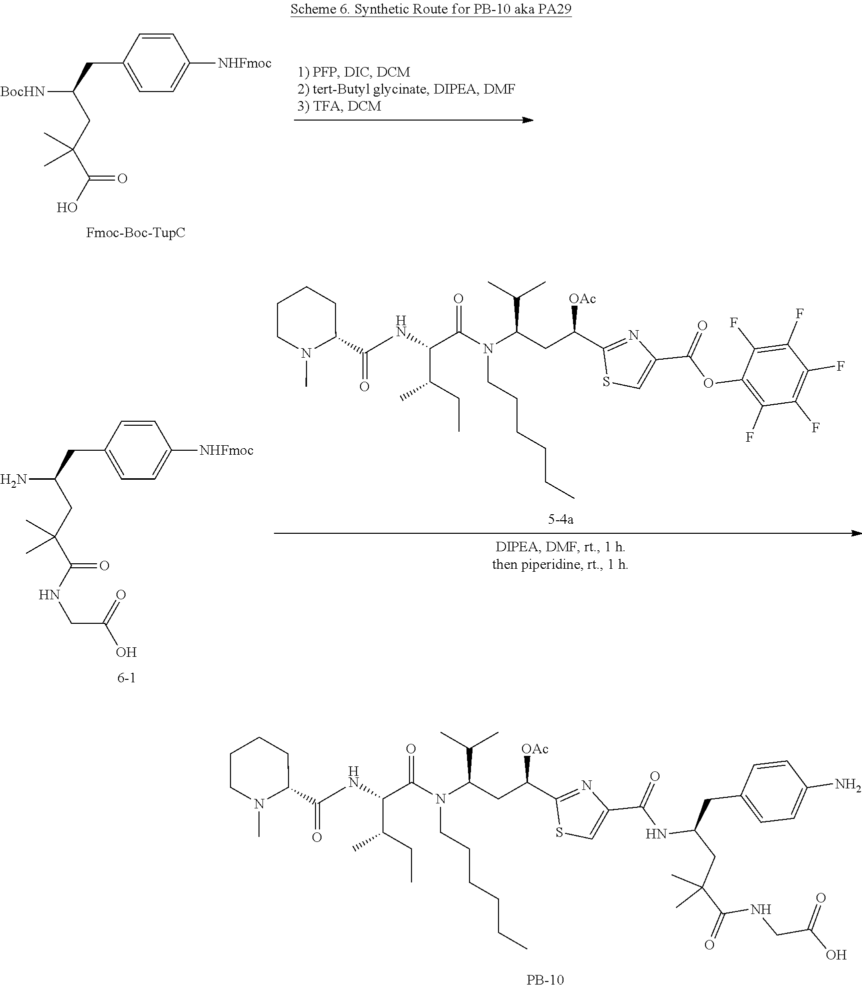

### PB-10 aka PA29

### Rac-(R)-(5-(4-(0(9H-fluoren-9-yl)methoxy)carbonyl)amino)phenyl)-4-amino-2,2-dimethylpentanoyl)glycine (6-1)

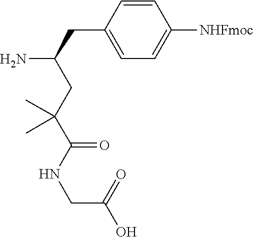

To a solution of compound Fmoc-Boc-TupC (0.20 g, 0.35 mmol) in DCM (5 mL) was added PFP (92 mg, 0.50 mmol) and DIC (63 mg, 0.50 mmol) and the reaction mixture was stirred at room temperature for an hour, which was monitored by LCMS. The resulting mixture was concentrated in vacuo and the residue (0.20 g, ESI m/z 747.3 (M+Na)⁺) was dissolved in DMF (4 mL). To the resulting solution was added tert-butyl glycinate (52 mg, 0.40 mmol) and DIPEA (52 mg, 0.40 mmol) and the reaction mixture was stirred at room temperature for an hour, which was monitored by LCMS. The resulting mixture was directly separated by reversed phase flash chromatography (0-100% acetonitrile in aq. ammonium bicarbonate (10 mM)) to give a white solid (0.14 g, ESI m/z 572.4 (M-Boc+H)⁺), which was dissolved in DCM (4 mL). To the solution was added TFA (2 mL) and the reaction mixture was stirred at room temperature for an hour, which was monitored by LCMS. The volatiles were removed in vacuo and the residue was purified by reversed phase flash chromatography (0-100% acetonitrile in aq. ammonium bicarbonate (10 mM)) to give compound 6-1 (90 mg, 50% yield) as a white solid. ESI m/z 516.3 (M+H)⁺.

### Rac-((R)-4-(2-((1S,3S)-1-acetoxy-3-((2R,3R)—N-hexyl-3-methyl-2-((S)-1-methylpiperidine-2-carboxamido)pentanamido)-4-methylpentyl)thiazole-4-carboxamido)-5-(4-aminophenyl)-2,2-dimethylpentanoyl)glycine (PB-10 aka PA29)

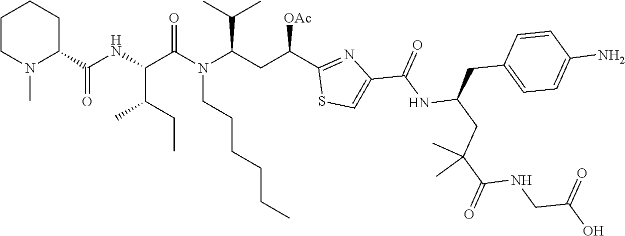 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)NCC(=O)O)cs1)C(C)C]

Following Alternative General Procedure VI (and then Fmoc removal) starting from PFP ester 5-4a (30 mg, 38 μmol) with aniline 6-1 (20 mg, 38 μmol), payload PB-10 aka PA29 (3.4 mg, 11% yield) was obtained as a white solid. ESI m/z 884.3 (M+H)⁺.

Scheme 7. Synthetic Route for Tubulysin-ether Analogues (PC-1 and PC-2 aka PA22 and PA23, respectively)

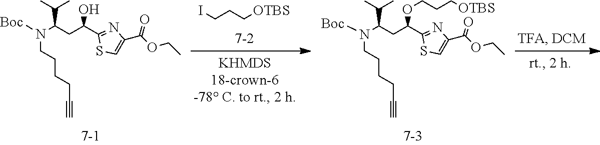 [SMILES: C#CCCCCN(C(=O)OC(C)(C)C)[C@H](C[C@@H](O)c1nc(C(=O)OCC)cs1)C(C)C ; CC(C)(C)[Si](C)(C)OCCCI ; C#CCCCCN(C(=O)OC(C)(C)C)[C@H](C[C@@H](OCCCO[Si](C)(C)C(C)(C)C)c1nc(C(=O)OCC)cs1)C(C)C]

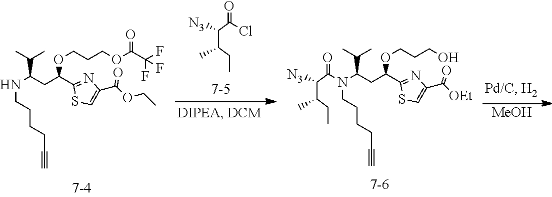 [SMILES: "MeOH" ; C#CCCCCN[C@H](C[C@@H](OCCCOC(=O)C(F)(F)F)c1nc(C(=O)OCC)cs1)C(C)C ; CC[C@H](C)[C@H](N=[N+]=[N-])C(=O)Cl ; C#CCCCCN(C(=O)[C@@H](N=[N+]=[N-])[C@@H](C)CC)[C@H](C[C@@H](OCCCO)c1nc(C(=O)OCC)cs1)C(C)C]

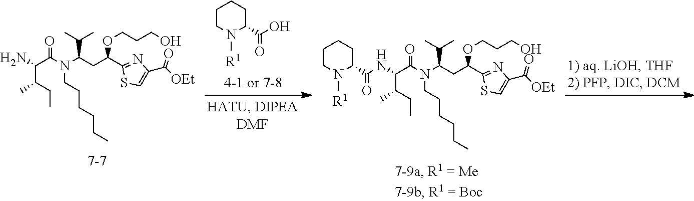

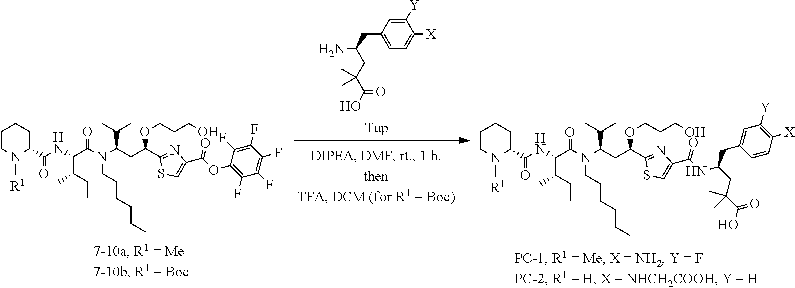

| | R¹ | X | Y |
|---|---|---|---|
| PC-1 aka PA22 | Me | NH₂ | F |
| PC-2 aka PA23 | H | NHCH₂COOH | H |

### Ethyl 2-((9R,11R)-12-(hex-5-yn-1-yl)-11-isopropyl-2,2,3,3,15,15-hexamethyl-13-oxo-4,8,14-trioxa-12-aza-3-silahexadecan-9-yl)thiazole-4-carboxylate (7-3)

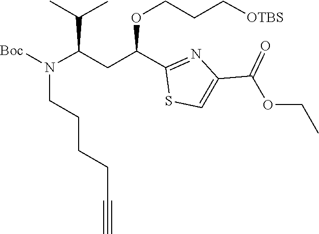 [SMILES: C#CCCCCN(C(=O)OC(C)(C)C)[C@H](C[C@@H](OCCCO[Si](C)(C)C(C)(C)C)c1nc(C(=O)OCC)cs1)C(C)C]

To a mixture of compound 7-1 (0.45 g, 1.0 mmol) and 18-crown-6 (0.79 g, 3.0 mmol) in anhydrous THF (20 mL) was added KHMDS (1 N in THF, 3 mL) at −78° C., and the resulting mixture was stirred at this temperature for an hour. To the cooled, stirred solution was added 0-TBS-3-iodopropanol (7-2) (1.2 g, 4.0 mmol) and the reaction mixture was allowed to warm to room temperature and was stirred at room temperature for two hours. The reaction mixture was then quenched with water (10 mL) and extracted with ethyl acetate (3×20 mL). The combined organic solution was concentrated in vacuo and the residue was purified by reversed phase flash chromatography (0-100% acetonitrile in aq. TFA (0.01%)) to give compound 7-3 (0.15 g, 24% yield) as a yellow oil. ESI m/z 625.4 (M+H)⁺.

### Ethyl 2-((1R,3R)-3-(hex-5-yn-1-ylamino)-4-methyl-1-(3-(2,2,2-trifluoroacetoxy)propoxy)pentyl)thiazole-4-carboxylate (7-4)

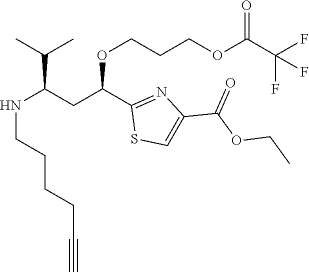 [SMILES: C#CCCCCN[C@H](C[C@@H](OCCCOC(=O)C(F)(F)F)c1nc(C(=O)OCC)cs1)C(C)C]

To a mixture of compound 7-3 (0.15 g, 0.24 mmol) in DCM (5 mL) was added TFA (1 mL), and the reaction mixture was stirred at room temperature for two hours. The volatiles were removed in vacuo to give compound 7-4 (0.10 g, 90% yield) as a yellow oil, which was used directly without further purification. ESI m/z 507.2 (M+H)⁺.

### Rac-ethyl 2-((1R,3R)-3-((2S,3S)-2-azido-N-(hex-5-yn-1-yl)-3-methylpentanamido)-1-(3-hydroxypropoxy)-4-methylpentyl)thiazole-4-carboxylate (7-6)

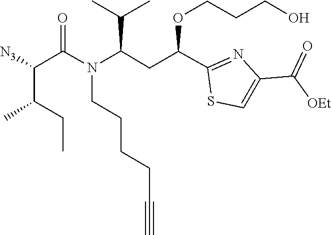 [SMILES: C#CCCCCN(C(=O)[C@@H](N=[N+]=[N-])[C@@H](C)CC)[C@H](C[C@@H](OCCCO)c1nc(C(=O)OCC)cs1)C(C)C]

To a mixture of compound 7-4 (0.10 g, 0.20 mmol) in DCM (5 mL) was added compound 7-5 (70 mg, 0.40 mmol) and DIPEA (50 mg, 0.40 mmol) and the reaction mixture was stirred at room temperature for two hours, which was monitored by LCMS. The resulting mixture was concentrated in vacuo and the residue was purified by silica gel flash chromatography (10-20% ethyl acetate in petroleumether) to give compound 7-6 (50 mg, 50% yield) as a yellow oil. ESI m/z 550.3 (M+H)⁺.

### Rac-ethyl 2-((1R,3R)-3-((2S,3S)-2-amino-N-hexyl-3-methylpentanamido)-1-(3-hydroxypropoxy)-4-methylpentyl)thiazole-4-carboxylate (7-7)

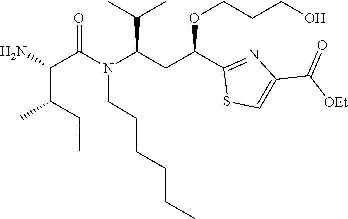 [SMILES: CCCCCCN(C(=O)[C@@H](N)[C@@H](C)CC)[C@H](C[C@@H](OCCCO)c1nc(C(=O)OCC)cs1)C(C)C]

To a solution of compound 7-6 (55 mg, 0.10 mmol) in methanol (10 mL) was added 10% palladium-carbon (10 mg) under nitrogen atmosphere and the reaction mixture was stirred at room temperature under a hydrogen balloon for two hours, which was monitored by LCMS. The resulting suspension was filtered through Celite and the filtrate was concentrated in vacuo to give compound 7-7 (50 mg, 90% yield) as a yellow oil. ESI m/z 528.4 (M+H)⁺.

### Ethyl 2-((1RS,3RS)-3-((2SR,3SR)—N-hexyl-3-methyl-2-((R)-1-methylpiperidine-2-carboxamido)pentanamido)-1-(3-hydroxypropoxy)-4-methylpentyl)thiazole-4-carboxylate (7-9a)

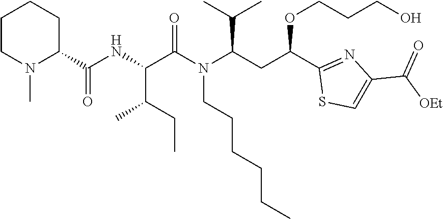 [SMILES: CCCCCCN(C(=O)[C@@H](NC(=O)[C@H]1CCCCN1C)[C@@H](C)CC)[C@H](C[C@@H](OCCCO)c1nc(C(=O)OCC)cs1)C(C)C]

Following Alternative General Procedure IIb starting from compound 7-7 (50 mg, 95 μmol) with compound 7-8 (17 mg, 0.12 mmol), compound 7-9a (60 mg, 90% yield) was obtained as a yellow oil without purification. ESI m/z 653.4 (M+H)⁺.

### Ethyl 24(1RS,3RS)-3-((2SR,3SR)-2-((R)-1-(tert-butoxycarbonyl)piperidine-2-carboxamido)-N-hexyl-3-methylpentanamido)-1-(3-hydroxypropoxy)-4-methylpentyl)thiazole-4-carboxylate (7-9b)

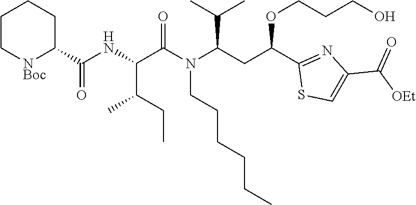 [SMILES: CCCCCCN(C(=O)[C@@H](NC(=O)[C@H]1CCCCN1C(=O)OC(C)(C)C)[C@@H](C)CC)[C@H](C[C@@H](OCCCO)c1nc(C(=O)OCC)cs1)C(C)C]

Following Alternative General Procedure IIb starting from compound 7-7 (50 mg, 95 μmol) with compound 4-1 (27 mg, 0.12 mmol), compound 7-9b (60 mg, 80% yield) was obtained as a yellow oil without purification. ESI m/z 739.2 (M+H)⁺.

### PC-1 aka PA22

### Rac-(R)-5-(4-amino-3-fluorophenyl)-4-(2-((1S,3S)-3-((2R,3R)—N-hexyl-3-methyl-2-((S)-1-methylpiperidine-2-carboxamido)pentanamido)-1-(3-hydroxypropoxy)-4-methylpentyl)thiazole-4-carboxamido)-2,2-dimethylpentanoic acid (PC-1 aka PA22)

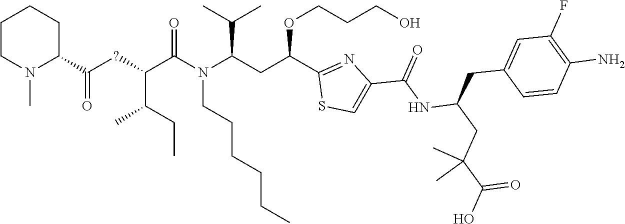

Successively following Alternative General Procedures III, V, and VI starting from 7-9a and treating with TupA in Alternative Procedure VI, payload PC-1 aka PA22 (10 mg, 13% yield) was obtained as a white solid. ESI m/z 861.3 (M+H)⁺. ¹H NMR (500 MHz, DMSO-d₆) δ 8.15 (s, 1H), 7.73 (s, 1H), 7.44 (s, 1H), 6.73 (d, J=13.0 Hz, 1H), 6.64-6.60 (m, 2H), 4.91 (s, 2H) 4.52 (t, J=9.4 Hz, 1H), 4.30 (d, J=12.2 Hz, 1H), 4.20 (s, 1H), 3.82-3.70 (m, 1H), 3.58-3.51 (m, 6H), 3.00-2.93 (m, 1H), 2.86-2.82 (m, 1H), 2.63-2.55 (m, 2H), 2.09 (s, 3H), 1.98-1.85 (m, 4H), 1.85-1.77 (m, 2H), 1.72-1.69 (m, 3H), 1.66-1.48 (m, 5H), 1.45-1.36 (m, 2H), 1.28-1.24 (m, 7H), 1.19-1.11 (m, 2H), 1.06 (s, 3H), 1.05 (s, 3H), 0.91-0.80 (m, 13H), 0.70 (s, 3H) ppm.

### PC-2 aka PA23

### Rac-(R)-5-(4-((carboxymethyl)amino)phenyl)-4-(2-((1S,3S)-3-((2R,3R)—N-hexyl-3-methyl-2-((S)-piperidine-2-carboxamido)pentanamido)-1-(3-hydroxypropoxy)-4-methylpentyl)thiazole-4-carboxamido)-2,2-dimethylpentanoic acid (PC-2 aka PA23)

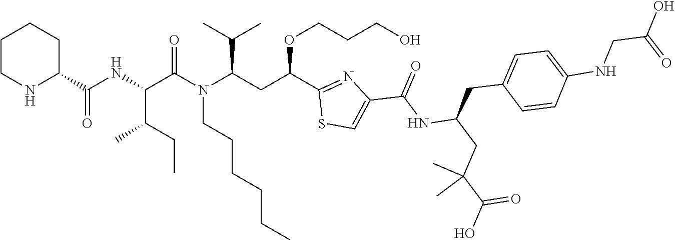 [SMILES: CCCCCCN(C(=O)[C@@H](NC(=O)[C@H]1CCCCN1)[C@@H](C)CC)[C@H](C[C@@H](OCCCO)c1nc(C(=O)N[C@@H](Cc2ccc(NCC(=O)O)cc2)CC(C)(C)C(=O)O)cs1)C(C)C]

Successively following Alternative General Procedures III, V, and VI (then Boc removal) starting from 7-9b and treating with 5-3b in Alternative General Procedure VI, payload PC-1 aka PA23 (7 mg, 5.5% yield) was obtained as a white solid. ESI m/z 887.2 (M+H)⁺. ¹H NMR (500 MHz, DMSO-d₆) δ 8.44 (d, J=12.7 Hz, 1H), 8.17 (s, 1H), 7.18 (s, 1H), 7.13 (s, 1H), 6.88 (d, J=8.1 Hz, 2H), 6.43 (d, J=8.2 Hz, 2H), 4.55-4.25 (m, 5H), 3.72-3.70 (m, 2H), 3.65-3.47 (m, 5H), 3.18-3.11 (m, 2H), 2.82-2.70 (m, 2H), 2.61-2.50 (m, 3H), 2.33 (s, 1H), 2.12 (s, 2H), 2.04-1.86 (m, 3H), 1.86-1.66 (m, 6H), 1.60-1.50 (m, 3H), 1.43-1.38 (m, 4H), 1.19-1.12 (m, 2H), 1.08-1.03 (m, 8H), 0.99 (d, J=6.2 Hz, 3H), 0.93-0.68 (m, 13H) ppm.

Scheme 8. Synthetic Route for Tubulysin-carbamate Analogues (PC-3, 4, 5, and
6, aka PA24, PA25, PA26, and PA27, respectively)

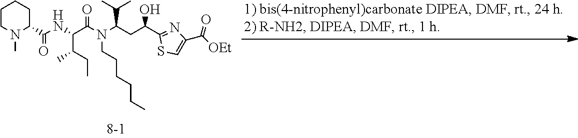 [SMILES: CCCCCCN(C(=O)[C@@H](NC(=O)[C@H]1CCCCN1C)[C@@H](C)CC)[C@H](C[C@@H](O)c1nc(C(=O)OCC)cs1)C(C)C]

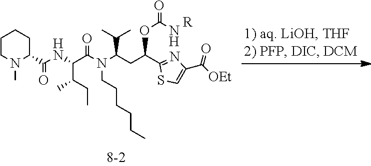

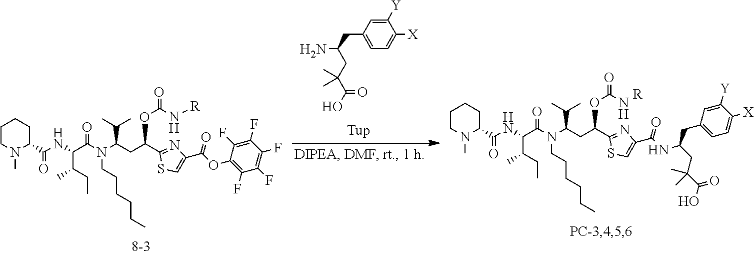

| | R | X | Y |
|---|---|---|---|
| PC-3 aka PA24 | CH₂CH₂OH | NH₂ | F |
| PC-4 aka PA25 | CH₂(CH₂OCH₂)₂CH₂OH | NH₂ | F |
| PC-5 aka PA26 | CH₂CH(OH)CH₂OH | NH₂ | H |
| PC-6 aka PA27 | CH₂CH(OH)CH₂OH | NHCH₂CH₂OH | H |

### Synthesis of Intermediate 8-2

### Ethyl 2-((3RS,6SR,8SR)-3-((RS)-sec-butyl)-5-hexyl-13-hydroxy-6-isopropyl-1-((R)-1-methylpiperidin-2-yl)-1,4,10-trioxo-9-oxa-2,5,11-triazatridecan-8-yl)thiazole-4-carboxylate (8-2a)

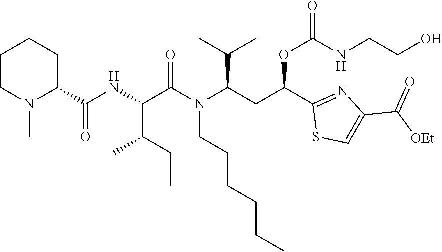 [SMILES: CCCCCCN(C(=O)[C@@H](NC(=O)[C@H]1CCCCN1C)[C@@H](C)CC)[C@H](C[C@@H](OC(=O)NCCO)c1nc(C(=O)OCC)cs1)C(C)C]

Following Alternative General Procedure VIII starting from 8-1 (80 mg, 0.13 mmol) with ethanolamine, compound 8-2a (20 mg, 22% yield) was obtained as a white solid. ESI m/z 682.3 (M+H)⁺.

Ethyl 2-((3RS,6SR,8SR)-3-((RS)-sec-butyl)-5-hexyl-19-hydroxy-6-isopropyl-1-((R)-1-methylpiperidin-2-yl)-1,4,10-trioxo-9,14,17-trioxa-2,5,11-triazanonadecan-8-yl)thiazole-4-carboxylate (8-2b)

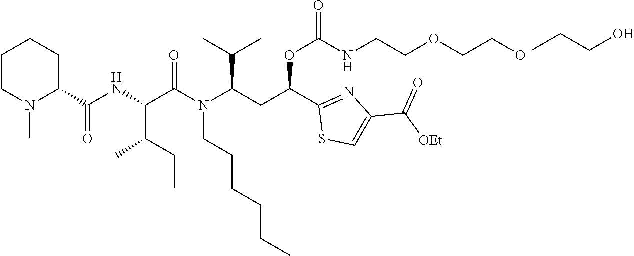

Following Alternative General Procedure VIII starting from 8-1 with 2-(2-(2-aminoethoxy)ethoxy)ethanol (CAS: 6338-55-2), compound 8-2b (91 mg, 35% yield) was obtained as a white solid. ESI m/z 770.2 (M+H)⁺.

### Ethyl 2-((3S,6R,8R)-3-((S)-sec-butyl)-5-hexyl-13,14-dihydroxy-6-isopropyl-1-((R)-1-methylpiperidin-2-yl)-1,4,10-trioxo-9-oxa-2,5,11-triazatetradecan-8-yl)thiazole-4-carboxylate (8-2c)

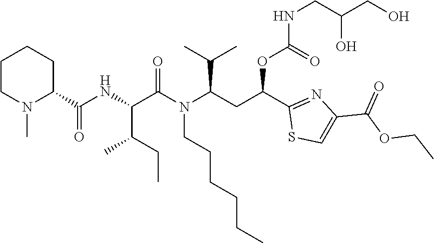 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)OCC)cs1)C(C)C]

Following Alternative General Procedure VIII starting from 8-1 with 3-aminopropane-1,2-diol (CAS: 616-30-8), compound 8-2b (0.16 g, 45% yield) was obtained as a white solid. ESI m/z 712.2 (M+H)⁺. ¹H NMR (400 MHz, DMSO-d₆) δ 8.47 (s, 1H), 7.60 (d, J=9.2 Hz, 1H), 7.41-7.36 (m, 1H), 5.55-5.50 (m, 1H), 4.62 (d, J=4.8 Hz, 1H), 4.50-4.46 (m, 1H), 4.40 (t, J=5.6 Hz, 1H), 4.32-4.28 (m, 2H), 3.72-3.64 (m, 1H), 3.51-3.45 (m, 1H), 3.14-3.03 (m, 1H), 3.00-2.88 (m, 2H), 2.85-2.79 (m, 1H), 2.46-2.43 (m, 1H), 2.15-2.10 (m, 1H), 2.06 (s, 3H), 1.96-1.87 (m, 3H), 1.64-1.57 (m, 3H), 1.55-1.49 (m, 2H), 1.47-1.43 (m, 1H), 1.34-1.27 (m, 11H), 1.15-1.07 (m, 2H), (m, 16H), 0.72-0.66 (m, 3H) ppm.

### Synthesis of Payloads (PC-3, 4, 5, and 6, aka PA24, PA25, PA26, and PA27, Respectively)

### PC-3 aka PA24

### (S)-5-(4-amino-3-fluorophenyl)-4-(2-((3RS,6SR,8SR)-3-((RS)-sec-butyl)-5-hexyl-13-hydroxy-6-isopropyl-1-((R)-1-methylpiperidin-2-yl)-1,4,10-trioxo-9-oxa-2,5,11-triazatridecan-8-yl)thiazole-4-carboxamido)-2,2-dimethylpentanoic acid (PC-3 aka PA24)

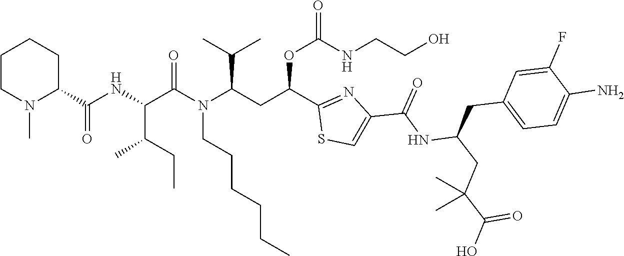 [SMILES: CCCCCCN(C(=O)[C@@H](NC(=O)[C@H]1CCCCN1C)[C@@H](C)CC)[C@H](C[C@@H](OC(=O)NCCO)c1nc(C(=O)N[C@@H](Cc2ccc(N)c(F)c2)CC(C)(C)C(=O)O)cs1)C(C)C]

Successively following Alternative General Procedures III, V, and VI starting from 8-2a (20 mg, 29 μmol) and treating with TupA in Alternative General Procedure VI, compound PC-3 aka PA24 (5.4 mg, 21% yield) was obtained as a white solid. ESI m/z 890.2 (M+H)⁺. ¹H NMR (400 MHz, DMSO-d₆) δ 8.13 (s, 1H), 7.62-7.37 (m, 3H), 6.75 (d, J=12.2 Hz, 1H), 6.68-6.60 (m, 2H), 5.59-5.51 (m, 1H), 4.92 (s, 2H), 4.62 (t, J=6.0 Hz, 1H), 4.48 (t, J=9.4 Hz, 1H), 4.26-4.19 (m, 1H), 3.76-3.65 (m, 1H), 3.58-3.48 (m, 1H), 3.23-3.12 (m, 2H), 3.07-2.96 (m, 3H), 2.60 (d, J=6.5 Hz, 2H), 2.21-2.05 (m, 4H), 2.05-1.95 (m, 2H), 1.94-1.73 (m, 5H), 1.71-1.52 (m, 5H), 1.51-1.38 (m, 3H), 1.33-1.19 (m, 9H), 1.07 (s, 3H), 1.06 (s, 3H), 0.95 (d, J=6.4 Hz, 3H), 0.89-0.77 (m, 9H), 0.74-0.65 (m, 3H) ppm.

### PC-4 aka PA25

### (S)-5-(4-amino-3-fluorophenyl)-4-(2-((3RS,6SR,8SR)-3-((RS)-sec-butyl)-5-hexyl-19-hydroxy-6-isopropyl-1-((R)-1-methylpiperidin-2-yl)-1,4,10-trioxo-9,14,17-trioxa-2,5,11-triazanonadecan-8-yl)thiazole-4-carboxamido)-2,2-dimethylpentanoic acid (PC-4 aka PA25)

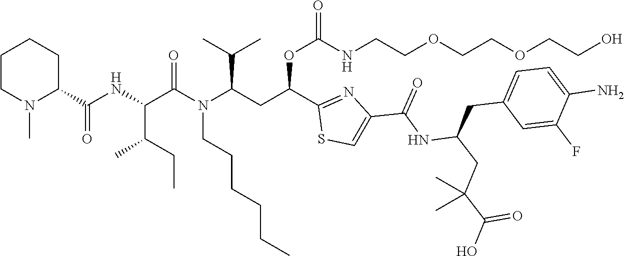 [SMILES: CCCCCCN(C(=O)[C@@H](NC(=O)[C@H]1CCCCN1C)[C@@H](C)CC)[C@H](C[C@@H](OC(=O)NCCOCCOCCO)c1nc(C(=O)N[C@@H](Cc2ccc(N)c(F)c2)CC(C)(C)C(=O)O)cs1)C(C)C]

Successively following Alternative General Procedures III, V, and VI starting from 8-2b (91 mg, 0.12 mmol) and treating with TupA in Alternative General Procedure VI, compound PC-4 aka PA25 (25 mg, 21% yield) was obtained as a white solid. ESI m/z 987.6 (M+H)⁺. ¹H NMR (400 MHz, DMSO-d₆) δ 9.72 (s, 1H), 9.12 (d, J=9.2 Hz, 1H), 8.16 (s, 1H), 7.62 (t, J=5.5 Hz, 1H), 6.77 (d, J=12.8 Hz, 1H), 6.71-6.60 (m, 2H), 5.61-5.51 (m, 1H), 4.57-4.46 (m, 2H), 4.30-4.18 (m, 2H), 3.45-3.26 (m, 12H), 3.18-2.96 (m, 5H), 2.71-2.58 (m, 5H), 2.23-2.05 (m, 3H), 2.00-1.73 (m, 6H), 1.71-1.52 (m, 4H), 1.48-1.15 (m, 9H), 1.15-1.02 (m, 6H), 0.95 (d, J=6.3 Hz, 3H), (m, 9H), 0.72 (s, 3H) ppm.

### PC-5 aka PA26

### (4S)-5-(4-aminophenyl)-4-(2-((3S,6R,8R)-3-((S)-sec-butyl)-5-hexyl-13,14-dihydroxy-6-isopropyl-1-((R)-1-methylpiperidin-2-yl)-1,4,10-trioxo-9-oxa-2,5,11-triazatetradecan-8-yl)thiazole-4-carboxamido)-2,2-dimethylpentanoic acid (PC-5 aka PA26)

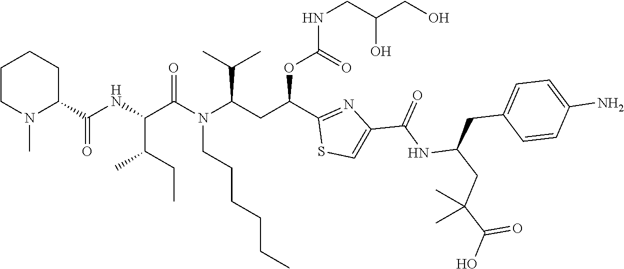 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(N)cc2)CC(C)(C)C(=O)O)cs1)C(C)C]

Successively following Alternative General Procedures III, V, and VI starting from 8-2c (0.11 g, 0.15 mmol) and treating with TupC in Alternative General Procedure VI, compound PC-5 aka PA26 (17 mg, 25% yield) was obtained as a white solid. ESI m/z 902.2 (M+H)⁺. ¹H NMR (400 MHz, DMSO-d₆) δ 8.13 (s, 1H), 7.76-7.60 (m, 1H), 7.44-7.40 (m, 1H), 6.80 (d, J=8.4 Hz, 2H), 6.45 (d, J=8.4 Hz, 2H), 5.58-5.50 (m, 1H), 4.92-4.60 (m, 3H), 4.52-4.40 (m, 2H), 4.19 (br s, 1H), 3.78-3.67 (m, 1H), 3.53-3.47 (m, 1H), 3.16-3.06 (m, 1H), 3.05-2.81 (m, 4H), 2.70-2.52 (m, 3H), 2.20-2.10 (m, 2H), 2.08 (s, 3H), 2.00-1.77 (m, 5H), 1.66-1.58 (m, 3H), 1.57-1.35 (m, 5H), 1.32-1.25 (m, 6H), 1.19-1.07 (m, 2H), 1.06-1.02 (m, 6H), 0.95 (d, J=6.4 Hz, 3H), 0.90-0.77 (m, 0.70 (s, 3H) ppm.

### PC-6 aka PA27

### (4S)-4-(2-03S,6R,8R)-3-((S)-sec-butyl)-5-hexyl-13,14-dihydroxy-6-isopropyl-1-((R)-1-methylpiperidin-2-yl)-1,4,10-trioxo-9-oxa-2,5,11-triazatetradecan-8-yl)thiazole-4-carboxamido)-5-(4-((2-hydroxyethyl)amino)phenyl)-2,2-dimethylpentanoic acid (PC-6 aka PA27)

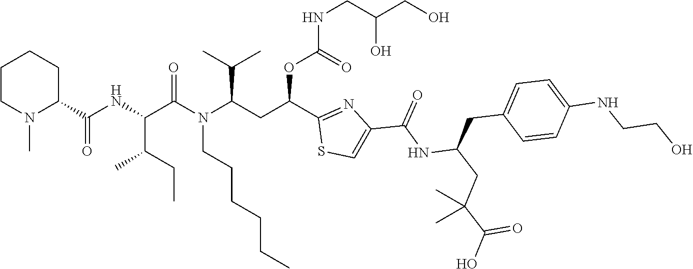 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(NCCO)cc2)CC(C)(C)C(=O)O)cs1)C(C)C]

Successively following Alternative General Procedures III, V, and VI starting from 8-2c (0.11 g, 0.15 mmol) and treating with 5-3a in Alternative General Procedure VI, compound PC-5 aka PA27 (19 mg, 26% yield) was obtained as a white solid. ESI m/z 946.5 (M+H)⁺. ¹H NMR (400 MHz, DMSO-d₆) δ 8.14 (s, 1H), 7.75-7.62 (m, 1H), 7.44-7.39 (m, 1H), 6.87 (d, J=8.4 Hz, 2H), 6.47 (d, J=8.0 Hz, 2H), 5.59-5.52 (m, 1H), 5.33-5.24 (m, 1H), 4.72-4.60 (m, 2H), 4.48 (t, J=9.6 Hz, 2H), 4.25-4.17 (m, 1H), 3.75-3.65 (m, 1H), 3.55-3.48 (m, 3H), 3.15-3.00 (m, 4H), 2.98-2.89 (m, 2H), 2.88-2.81 (m, 1H), 2.68-2.55 (m, 3H), 2.20-2.05 (m, 5H), 2.00-1.80 (m, 5H), 1.67-1.57 (m, 4H), 1.56-1.49 (m, 2H), 1.43-1.35 (m, 2H), 1.32-1.25 (m, 6H), 1.23-1.08 (m, 3H), 1.07-1.01 (m, 7H), 0.95 (d, J=6.4 Hz, 3H), 0.90-0.79 (m, 10H), 0.70 (s, 3H) ppm.

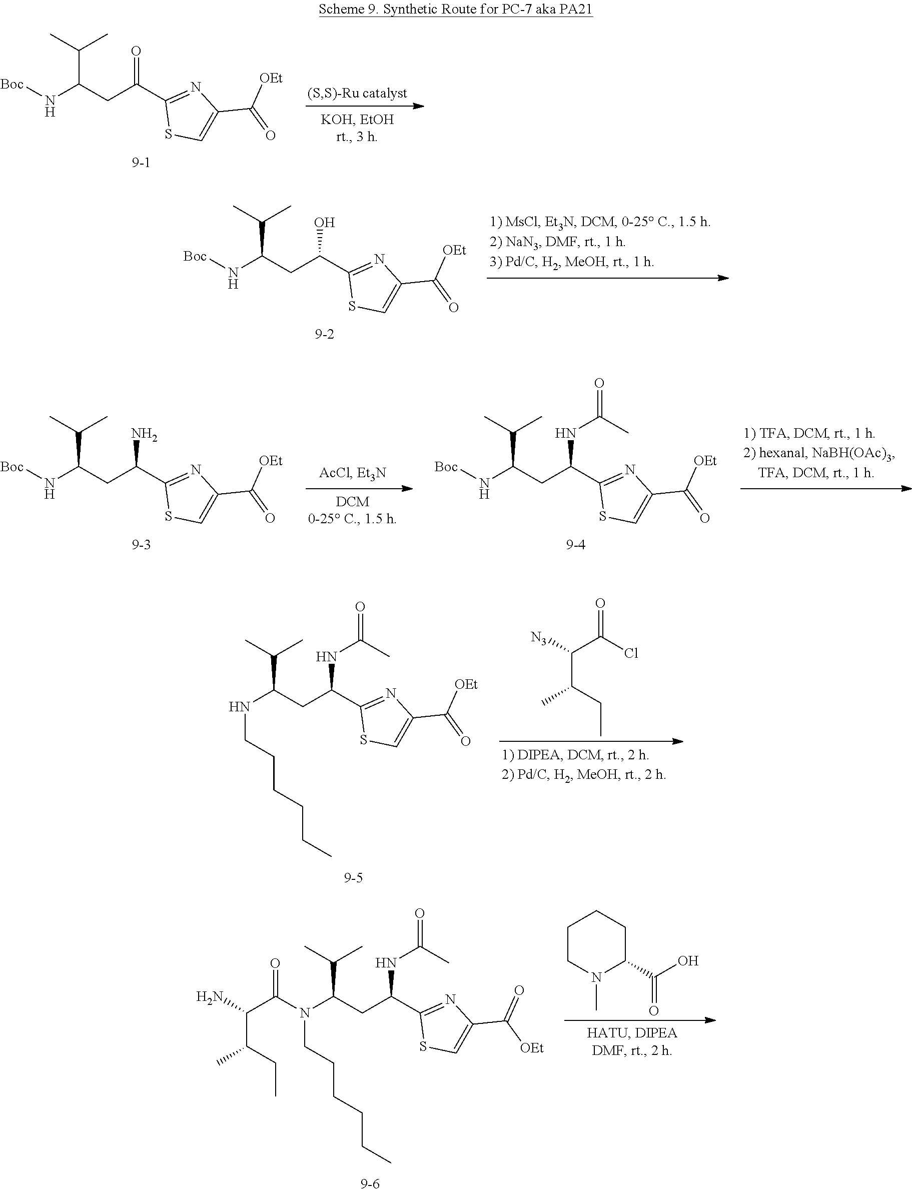

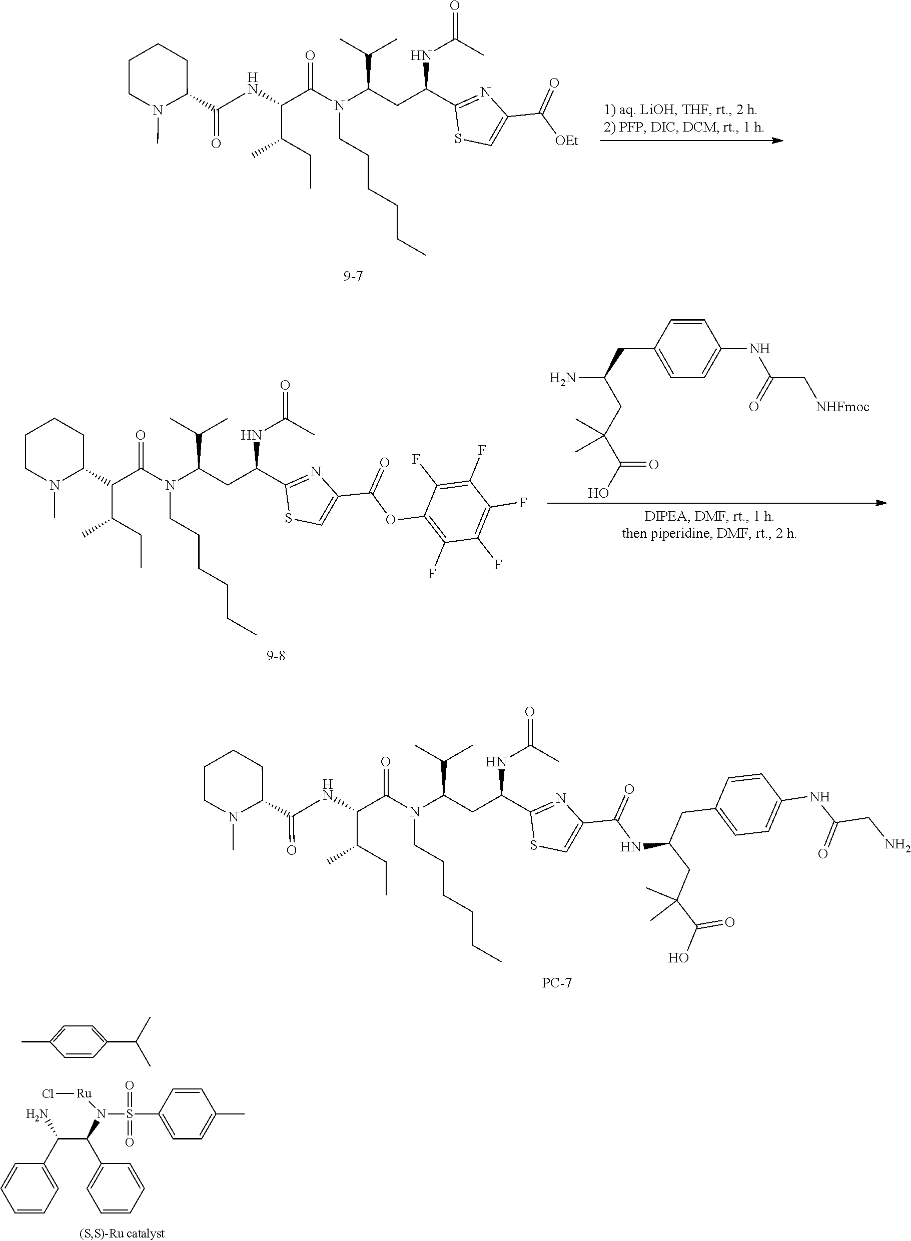

### PC-7 aka PA21

### Ethyl 24(1S,3R)-3-((tert-butoxycarbonyl)amino)-1-hydroxy-4-methylpentyl)thiazole-4-carboxylate (9-2)

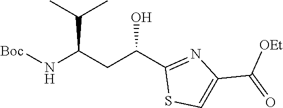 [SMILES: CCOC(=O)c1csc([C@@H](O)C[C@@H](NC(=O)OC(C)(C)C)C(C)C)n1]

To a solution of compound 9-1 (CAS: 944559-47-1, 3.7 g, 10 mmol) in ethanol was added potassium hydroxide (56 mg, 1.0 mmol) and (S,S)-Ru-catalyst (0.32 g, 0.50 mmol) and the reaction mixture was stirred at room temperature for three hours, which was monitored by LCMS. The resulting mixture was quenched with sat. aq. ammonium chloride (10 mL) and extracted with ethyl acetate (3×20 mL). The combined organic solution was washed with brine (20 mL), dried over anhydrous sodium sulfate, filtered, and concentrated in vacuo. The crude product was purified by silica gel column chromatography (15-20% ethyl acetate in petroleumether) to give compound 9-2 (1.7 g, 50% yield) as a colorless oil. ESI m/z 373.1 (M+H)⁺.

### Ethyl 2-((1R,3R)-1-amino-3-((tert-butoxycarbonyl)amino)-4-methylpentyl)thiazole-4-carboxylate (9-3)

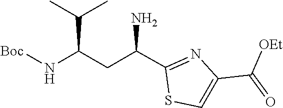 [SMILES: CCOC(=O)c1csc([C@H](N)C[C@@H](NC(=O)OC(C)(C)C)C(C)C)n1]

To a stirred suspension of compound 9-2 (1.4 g, 3.8 mmol) in DCM (50 mL) was successively added triethylamine (0.60 g, 5.9 mmol) and MsCl (0.55 g, 4.8 mmol) at 0° C. After the reaction mixture turned clear, the reaction mixture was stirred at 0° C. for an hour and then at room temperature for thirty minutes, which was monitored by LCMS. The resulting solution was successively washed with aq. HCl (1 N, 50 mL), water (50 mL), aq. sodium carbonate (10%, 50 mL), and brine (50 mL). The organic solution was dried over anhydrous sodium sulfate, filtered, and concentrated in vacuo to give a yellow oil (1.6 g, ESI m/z 451.0 (M+H)⁺, which was dissolved in DMF (10 mL). To the solution was added sodium azide (1.2 g, 18 mmol) and the mixture was stirred at room temperature for an hour. The resulting mixture was diluted with ethyl acetate (50 mL) and successively washed with water (10 mL) and brine (10 mL), dried over anhydrous sodium sulfate, filtered, and concentrated in vacuo to give a yellow oil (1.3 g, ESI m/z 398.1 (M+H)⁺), which was dissolved in methanol (50 mL). To the resulting solution was added palladium on carbon (10% Pd, 120 mg), and the reaction mixture was stirred at room temperature under a hydrogen atmosphere for an hour, which was monitored by LCMS. The resulting mixture was filtered through Celite and the filtrate was concentrated in vacuo to give compound 9-3 (1.0 g, 74% yield from 9-2) as a yellow oil. ESI m/z 372.1 (M+H)⁺.

### Ethyl 2-((1R,3R)-1-acetamido-3-((tert-butoxycarbonyl)amino)-4-methylpentyl)thiazole-4-carboxylate (9-4)

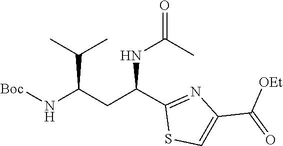 [SMILES: CCOC(=O)c1csc([C@@H](C[C@@H](NC(=O)OC(C)(C)C)C(C)C)NC(C)=O)n1]

To a stirred solution of compound 9-3 (1.1 g, 3.0 mmol) in DCM (50 mL) was added triethylamine (0.45 g, 4.5 mmol) and acetochloride (0.28 g, 3.6 mmol) at 0° C. After the reaction mixture turned clear, the reaction mixture was stirred at 0° C. for an hour and then at room temperature for thirty minutes. The resulting solution was successively washed with aq. HCl (1 N, mL), aq. sodium carbonate (10%, 50 mL), and brine (50 mL). The organic solution was dried over anhydrous sodium sulfate, filtered, and concentrated in vacuo. The crude product was purified by silica gel column chromatography (15-20% ethyl acetate in petroleumether) to give compound 9-4 (1.0 g, 90% yield) as a colorless oil. ESI m/z 414.3 (M+H)⁺.

Ethyl 2-((1R,3R)-1-acetamido-3-(hexylamino)-4-methylpentyl)thiazole-4-carboxylate (9-5)

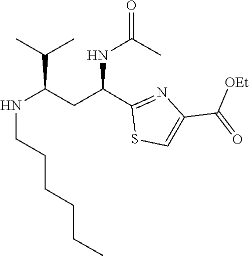 [SMILES: CCCCCCN[C@H](C[C@@H](NC(C)=O)c1nc(C(=O)OCC)cs1)C(C)C]

To a solution of compound 9-4 (1.3 g, 3.1 mmol) in DCM (20 mL) was added TFA (4 mL), and the reaction mixture was stirred at room temperature for an hour until Boc was totally removed according to LCMS. The volatiles were removed in vacuo to give a yellow solid (1.0 g, 314.2 (M+H)⁺), 0.7 g of which was suspended in DCM (30 mL). To the solution was added hexanal (0.26 g, 2.6 mmol), triacetoxyborohydride (0.70 g, 3.3 mmol), and TFA (two drops), and the mixture was stirred at room temperature for an hour, which was monitored by LCMS. The resulting mixture was successively washed with aq. HCl (1 N, 10 mL), water (10 mL), aq. sodium carbonate (10%, 10 mL), and brine (10 mL), dried over anhydrous sodium sulfate, filtered, and concentrated in vacuo. The residue was purified by chiral HPLC to give compound 9-5 (0.52 g, 54% yield) as a colorless oil. ESI m/z 398.3 (M+H)⁺.

### Rac-ethyl 2-((1R,3R)-1-acetamido-3-((2S,3S)-2-amino-N-hexyl-3-methylpentanamido)-4-methylpentyl)thiazole-4-carboxylate (9-6)

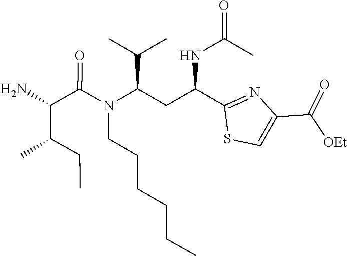 [SMILES: CCCCCCN(C(=O)[C@@H](N)[C@@H](C)CC)[C@H](C[C@@H](NC(C)=O)c1nc(C(=O)OCC)cs1)C(C)C]

Following similar procedures as 7-6 and 7-7 except starting from 9-5 (0.20 g, 0.50 mmol) instead of compound 7-4, compound azido-9-6 (0.15 g, ESI m/z 511.2 (M+H)⁺) was obtained as a yellow oil, which was dissolved in methanol (10 mL). To the resulting solution was added palladium-carbon (10%, 20 mg) under a nitrogen atmosphere and the reaction mixture was then stirred under a hydrogen atmosphere at room temperature for two hours until azide was totally reduced to amine according to LCMS. The resulting suspension was filtered through Celite and the filtrate was concentrated in vacuo. The crude product was purified by silica gel flash chromatography (50% ethyl acetate in petroleumether) to give compound 9-6 (0.14 g, 57% yield) as a yellow oil. ESI m/z 511.2 (M+H)⁺.

### Ethyl 2-((1RS,3RS)-1-acetamido-3-((2SR,3SR)—N-hexyl-3-methyl-2-((R)-1-methylpiperidine-2-carboxamido)pentanamido)-4-methylpentyl)thiazole-4-carboxylate (9-7)

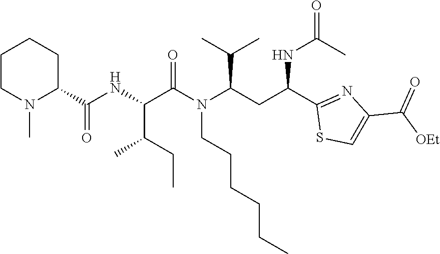 [SMILES: CCCCCCN(C(=O)[C@@H](NC(=O)[C@H]1CCCCN1C)[C@@H](C)CC)[C@H](C[C@@H](NC(C)=O)c1nc(C(=O)OCC)cs1)C(C)C]

Following Alternative General Procedure IIb starting from 9-6 (25 mg, 49 μmol) and treating with compound 7-8, compound 9-7 (27 mg, 87% yield) was obtained as a white solid. ESI m/z 636.5 (M+H)⁺.

### (S)-4-(2-((1R,3R)-1-acetamido-3-((2S,3S)—N-hexyl-3-methyl-2-((R)-1-methylpiperidine-2-carboxamido)pentanamido)-4-methylpentyl)thiazole-4-carboxamido)-acid (PC-7)

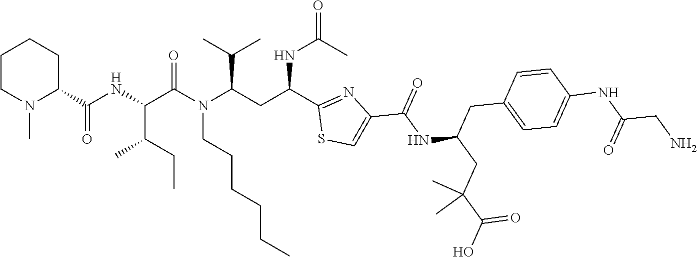 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(NC(=O)CN)cc2)CC(C)(C)C(=O)O)cs1)C(C)C]

Successively following Alternative General Procedures III and V starting from 9-7 (27 mg, 43 μmol), compound 9-8 (25 mg, ESI m/z 774.4 (M+H)⁺) was obtained as a yellow oil. Then following Alternative General Procedure VI (followed by Fmoc removal) from 9-8 (25 mg) and treating with Fmoc-TupD, payload PC-7 aka PA21 (13 mg, 34% yield from 9-7) was obtained as a white solid. ESI m/z 883.3 (M+H)⁺. ¹H NMR (400 MHz, DMSO-d₆) δ 8.69-8.62 (m, 1H), 8.06 (s, 1H), 7.53 (d, J=8.3 Hz, 2H), 7.10 (d, J=8.4 Hz, 2H), 4.87-4.78 (m, 1H), 4.45 (dd, J=14.3, 4.9 Hz, 1H), 4.27-4.16 (m, 1H), 3.74-3.56 (m, 2H), 3.22 (s, 4H), 3.02-2.63 (m, 7H), 2.11-2.02 (m, 4H), 1.98-1.76 (m, 8H), 1.65-1.31 (m, 9H), 1.25 (s, 3H), 1.22 (s, 3H), 1.19-1.08 (m, 2H), 1.07-0.90 (m, 10H), 0.89-0.76 (m, 9H), 0.74-0.64 (m, 3H) ppm.

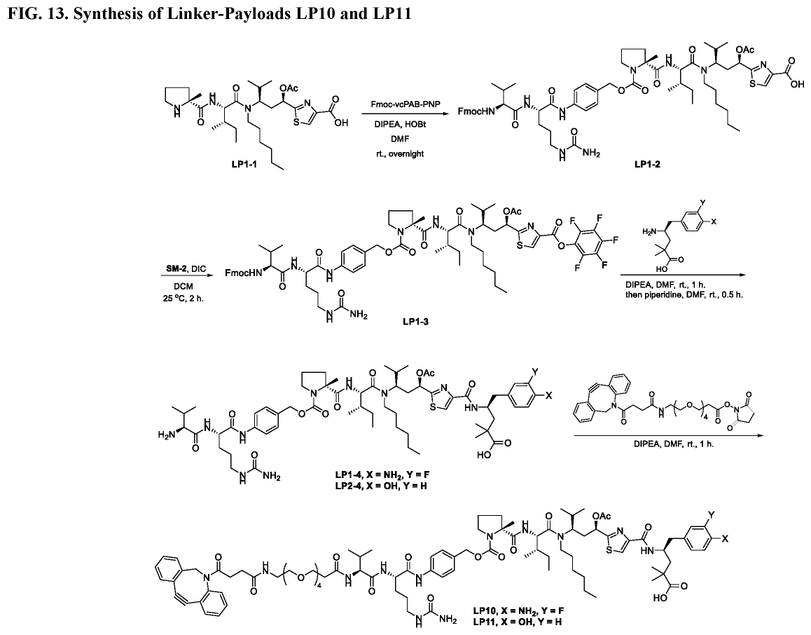

Synthesis of vcPAB-Linker-payloads LP1-4 and LP2-4 was consistent with FIG. 13.

Synthesis of LP1-4 and LP2-4 was consistent with the scheme below.

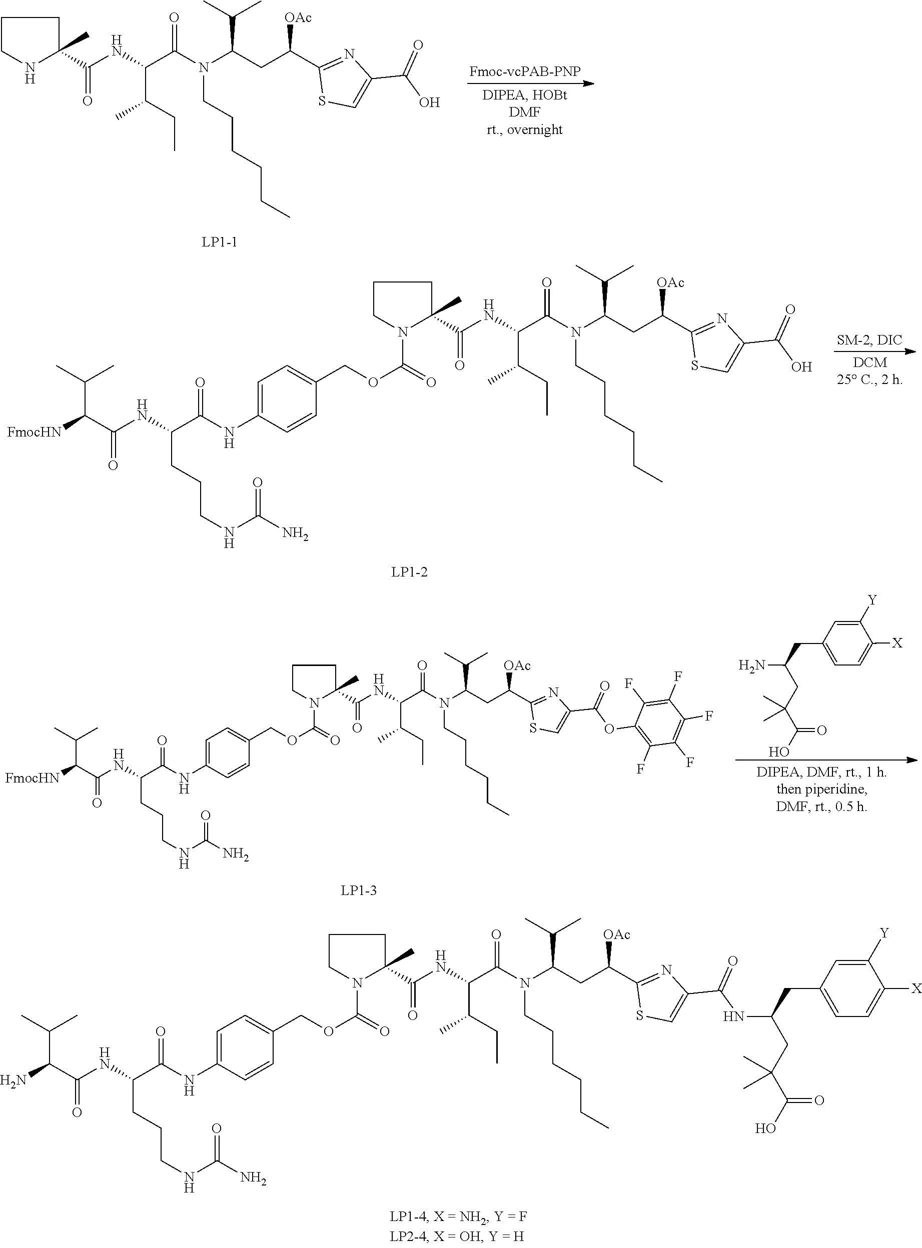

### General Procedure X

### Synthesis of LP1-4 and LP2-4

To a solution of acid LP1-2 (1.0 equiv) in DCM (30 mM) was added pentafluorophenol (SM-2) (2.5 equiv) and N,N′-diisopropylcarbodiimide (DIC) (2.5 equiv). The reaction mixture was stirred at room temperature for two hours, and monitored by LCMS. The resulting mixture was concentrated in vacuo to give the corresponding pentafluorophenol ester LP1-3, which is added into a mixture of

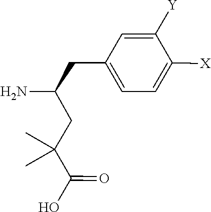

(1.0 equiv) and DIPEA (3.0 equiv) in DMF (15 mM). The reaction mixture was stirred at room temperature overnight, and monitored by LCMS. The resulting mixture was purified directly by reversed phase flash chromatography (0-100% acetonitrile in water) and the intermediate was dissolved in DMF (40 mM). To the solution was added piperidine (3.0 equiv) and the mixture was stirred at room temperature for two hours until Fmoc was totally removed according to LCMS. The resulting mixture was purified directly by reverse phase flash chromatography (0-100% acetonitrile in aq. TFA (0.01%)) to give compound LP1-4 and LP2-4 (over 3 steps from acid LP1-1).

### General Procedure XI

Amidation From Amines With —OSu Esters was consistent with the scheme below.

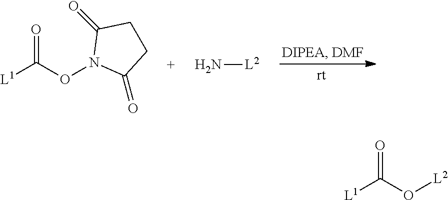

To a solution of amine (L²-NH₂) (1.0 equiv) in DMF (10 mM) is added -OSu ester (L¹-COOSu) (1.2-1.3 equiv) and DIPEA (2.5-3.0 equiv). The reaction solution was stirred at room temperature for two hours, and monitored by LCMS. The resulting solution was purified directly by reverse phase flash chromatography (0-100% acetonitrile in aq. ammonium bicarbonate (10 mM)) to give amide (L¹-CONH-L²).

### General Procedure XII

Synthesis of Carbamates From Amines with vcPAB-PNP Esters was consistent with the scheme below.

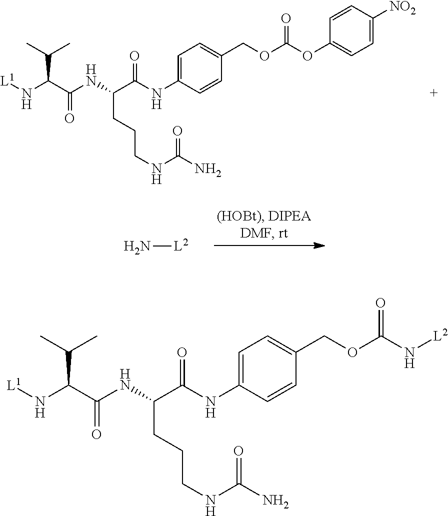

To a solution of amine (L²-NH₂) (1.0 equiv) in DMF (16 mM) is added L¹-vcPABC-PNP (1.0 equiv), HOBt (1.0 equiv or without HOBt), and DIPEA (3.0 equiv). The mixture was stirred at room temperature for one to four hours, and monitored by LCMS. The reaction mixture was purified directly by reverse phase flash chromatography (0-100% acetonitrile in aq. ammonium bicarbonate (10 mM)) to give the desired carbamates.

Synthesis of LP4-4 using General Procedure VII was consistent with the scheme below.

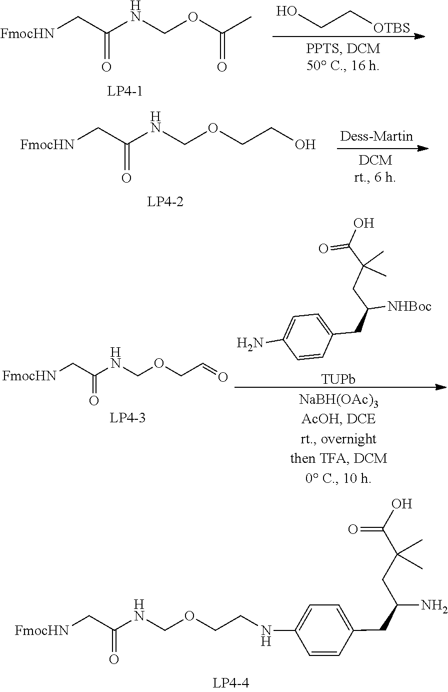

Synthesis of LP4-5 and LP5-5 followed from LP4-4 using General Procedure VII and was consistent with the scheme below.

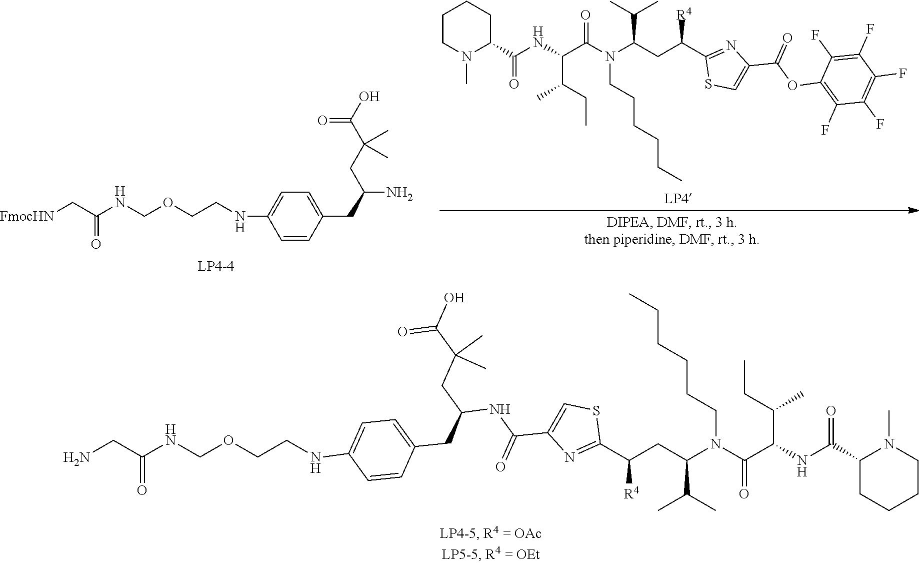

TABLE 4
Structures of Linker-payloads.

| | | Linker |
|---|---|---|
| | | Payload |
| LP# | Structures | name |
| LP1 | 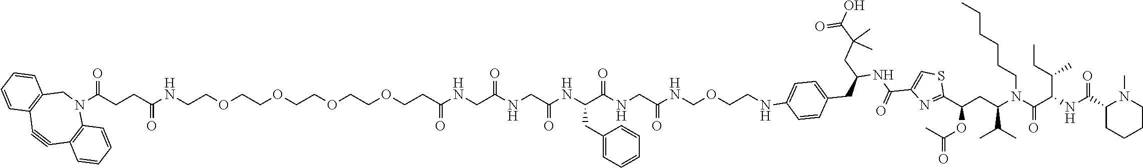 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCOCNC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C] | DIBAC- SUC- PEG4- GGFG- NHCH2- PA14 |
| LP2 | 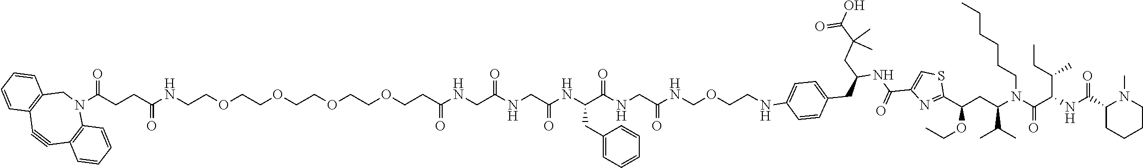 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCOCNC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C] | DIBAC- SUC- PEG4- GGFG- NH- CH2- PA15 |
| LP3 | 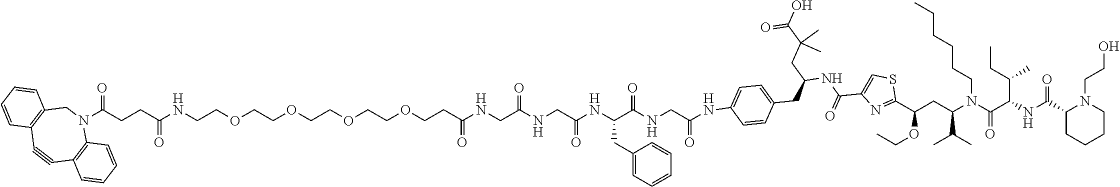 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CNC(=O)[C@H](Cc3ccccc3)NC(=O)CNC(=O)CNC(=O)CCOCCOCCOCCOCCNC(=O)CCC(=O)N3Cc4ccccc4C#Cc4ccccc43)cc2)CC(C)(C)C(=O)O)cs1)C(C)C] | DIBAC- SUC- PEG4- GGFG- PA13 |
| LP4 | 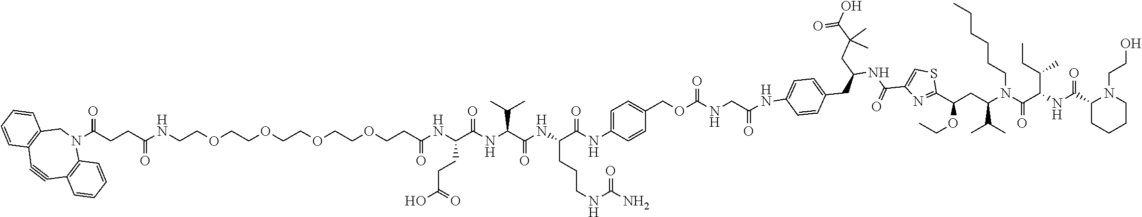 [SMILES: CCCCCCN(C(=O)[C@@H](NC(=O)[C@H]1CCCCN1CCO)[C@@H](C)CC)[C@H](C[C@@H](OCC)c1nc(C(=O)N[C@@H](Cc2ccc(NC(=O)CNC(=O)OCc3ccc(NC(=O)[C@H](CCCNC(N)=O)NC(=O)[C@@H](NC(=O)[C@H](CCC(=O)O)NC(=O)CCOCCOCCOCCOCCNC(=O)CCC(=O)N4Cc5ccccc5C#Cc5ccccc54)C(C)C)cc3)cc2)CC(C)(C)C(=O)O)cs1)C(C)C] | DIBAC- SUC- PEG4- EVC- PAB-G- PA13 |
| LP5 | 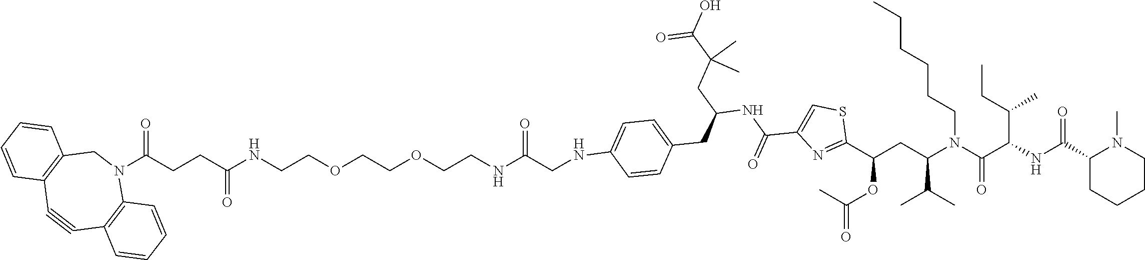 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCNC(=O)CCC(=O)N3Cc4ccccc4C#Cc4ccccc43)cc2)CC(C)(C)C(=O)O)cs1)C(C)C] | DIBAC- SUC- PEG2- PA16 |
| LP6 | 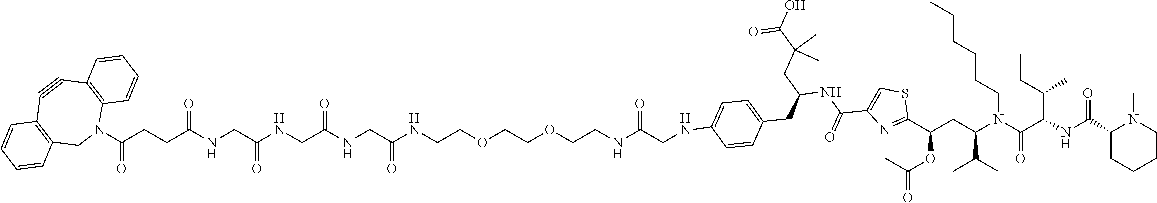 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCNC(=O)CNC(=O)CNC(=O)CNC(=O)CCC(=O)N3Cc4ccccc4C#Cc4ccccc43)cc2)CC(C)(C)C(=O)O)cs1)C(C)C] | DIBAC- SUC- GGG- PEG2- PA16 |
| LP7 | 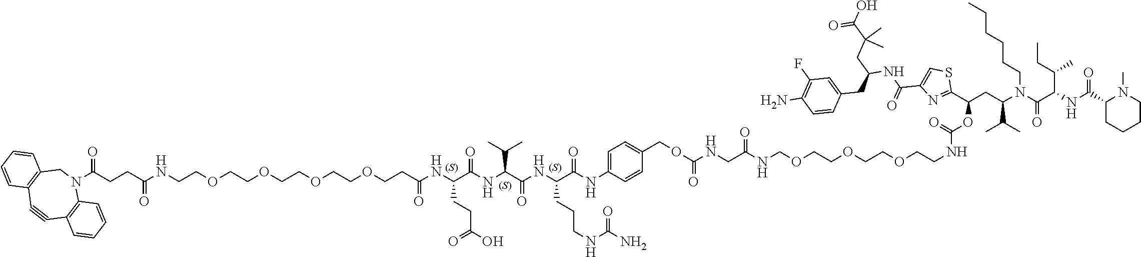 | DIBAC- SUC- PEG4- EvcPAB- G- NHCH2- PA25 |
| LP8 | 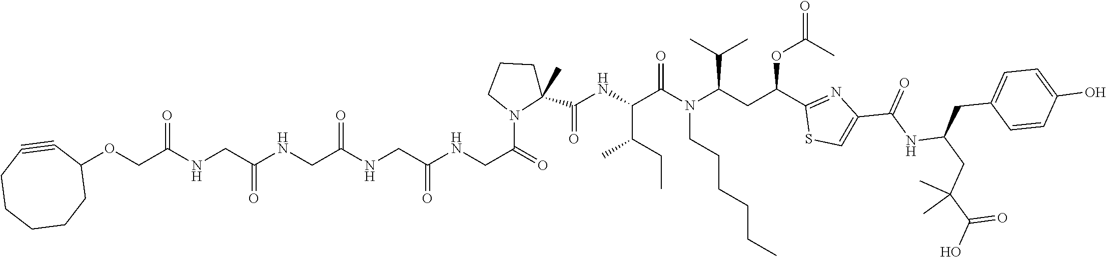 [SMILES: CCCCCCN(C(=O)[C@@H](NC(=O)[C@@]1(C)CCCN1C(=O)CNC(=O)CNC(=O)CNC(=O)CNC(=O)COC1C#CCCCCC1)[C@@H](C)CC)[C@H](C[C@@H](OC(C)=O)c1nc(C(=O)N[C@@H](Cc2ccc(O)cc2)CC(C)(C)C(=O)O)cs1)C(C)C] | COT- GGGG- P22 |
| LP9 | 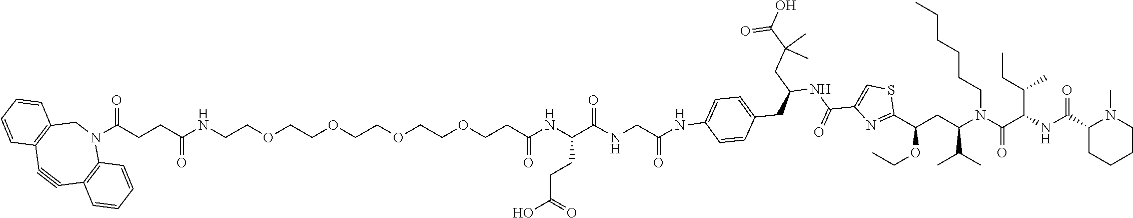 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CNC(=O)[C@H](CCC(=O)O)NC(=O)CCOCCOCCOCCOCCNC(=O)CCC(=O)N3Cc4ccccc4C#Cc4ccccc43)cc2)CC(C)(C)C(=O)O)cs1)C(C)C] | DIBAC- PEG4-E- P31 |
| LP10 | 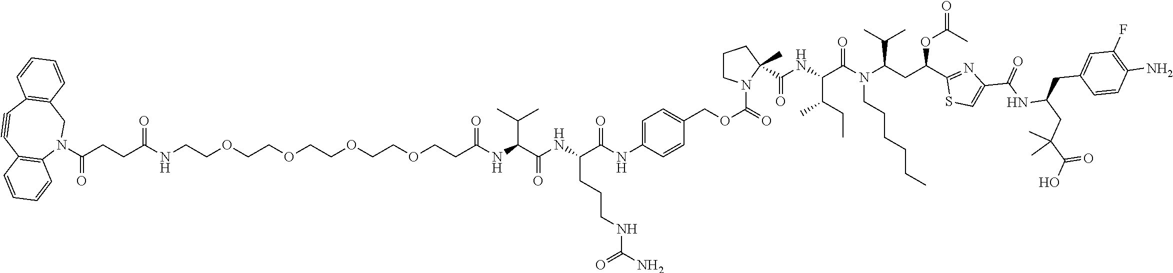 [SMILES: CCCCCCN(C(=O)[C@@H](NC(=O)[C@@]1(C)CCCN1C(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] | DIBAC- PEG4- vcPAB- P15 |
| LP11 | 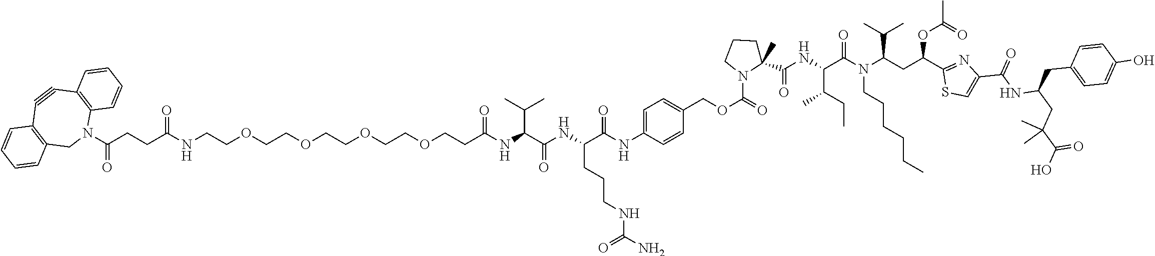 [SMILES: CCCCCCN(C(=O)[C@@H](NC(=O)[C@@]1(C)CCCN1C(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1)[C@@H](C)CC)[C@H](C[C@@H](OC(C)=O)c1nc(C(=O)N[C@@H](Cc2ccc(O)cc2)CC(C)(C)C(=O)O)cs1)C(C)C] | DIBAC- PEG4- vcPAB- P22 |

### (2S)-2-[(2S)-2-[(2S)-5-(tert-Butoxy)-2-{[(9H-fluoren-9-ylmethoxy)carbonyl]amino}-5-oxopentanamido]-3-methylbutanamido]-5-(carbamoylamino)pentanoic acid (L1-1a)

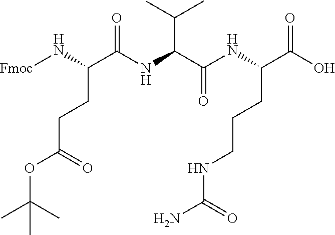

Following General Procedure XI using H-Val-Cit-OH (0.73 g, 2.1 mmol) with Fmoc-Glu(O^{t}Bu)-OSu (1.2 g, 2.3 mmol) provides Fmoc-Glu(O^{t}Bu)-Val-Cit-OH (L1-1a). ESI m/z: 682 (M+H)⁺.

### tert-Butyl (4S)-4-{[(1S)-1-{[(1S)-4-(carbamoylamino)-1-[(4-{[(4-nitrophenoxycarbonyl)oxy]methyl}phenyl)carbamoyl]butyl]carbamoyl}-2-methylpropyl]carbamoyl}-4-{[(9H-fluoren-9-ylmethoxy)carbonyl]amino}butanoate (L1-1c)

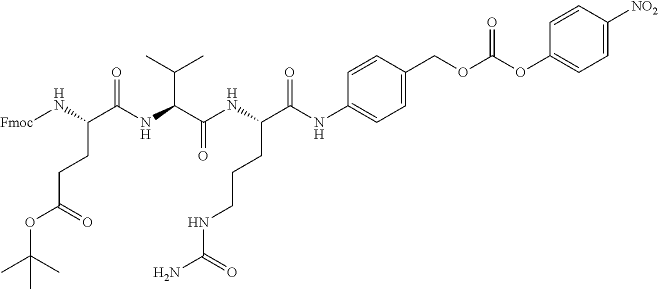

To a solution of Fmoc-Glu(O^{t}Bu)-OH (0.56 g, 1.3 mmol) in DMF (5 mL) was added HATU (0.50 g, 1.3 mmol) and DIPEA (0.34 g, 2.6 mmol). The reaction mixture was stirred at room temperature for ten minutes before the addition of vcPAB (0.50 g, 1.3 mmol). The mixture was stirred at room temperature for an hour and monitored by LCMS. The resulting mixture was purified by reverse phase flash chromatography (0-100% acetonitrile in aq. ammonium bicarbonate (10 mM)) to give Fmoc-Glu-Val-Cit-PABC (ESI m/z: 787 (M+H)⁺). Fmoc-Glu-Val-Cit-PABC was dissolved in DMF (5 mL). To the solution was added bis(4-nitrophenyl)carbonate (0.52 g, 1.7 mmol), DMAP (0.16 g, 1.3 mmol), and DIPEA (0.84 g, 6.5 mmol). The reaction mixture was stirred at room temperature for an hour, and monitored by LCMS. The resulting mixture was purified by reverse phase flash chromatography (0-100% acetonitrile in water) to give compound L1-1c. ESI m/z: 952 (M+H)⁺.

### (4S)-4-Amino-5-{4-[(2S)-5-(carbamoylamino)-2-[(2S)-2-[(2S)-4-carboxy-2-{[(9H-fluoren-9-ylmethoxy)carbonyl]amino}butanamido]-3-methylbutanamido]pentanamido]phenyl}-2,2-dimethylpentanoic acid (L1-2a)

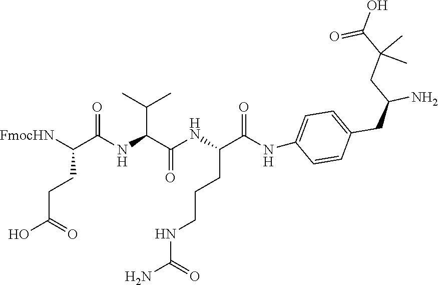

To a solution of Fmoc-Glu(O^{t}Bu)-Val-Cit-OH (L1-1a) (0.60 g, 0.88 mmol) in methanol (15 mL) was added EEDQ (0.23 g, 0.93 mmol) and TUP-6b (0.61 g, 1.8 mmol). The reaction mixture was stirred at 50° C. for four hours and monitored by LCMS. The resulting mixture was filtered and the filtrate was concentrated in vacuo. The residue (0.80 g) was dissolved in DCM (9 mL). To the solution was added TFA (3 mL), and the mixture was stirred at room temperature for two hours until both Boc and ^{t}Bu were totally removed according to LCMS. The resulting mixture was concentrated in vacuo and the residue was purified by reversed phase flash chromatography (0-40% acetonitrile in aq. ammonium bicarbonate (10 mM)) to give L1-2a. ESI m/z: 844 (M+H)⁺.

### (4S)-4-Amino-5-(4-{[({4-[(2S)-5-(carbamoylamino)-2-[(2S)-2-{[(9H-fluoren-9-ylmethoxy)carbonyl]amino}-3-methylbutanamido]pentanamido]phenyl}methoxy)carbonyl]amino}phenyl)-2,2-dimethylpentanoic acid (L1-2b)

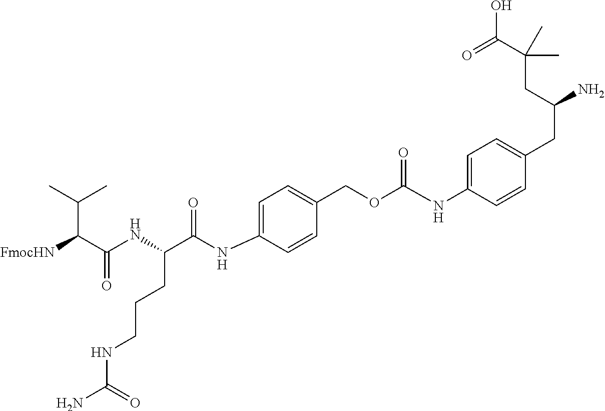

Following General Procedure XII using Fmoc-vcPABC-PNP (L1-1b) (50 mg, 65 μmol) and amine TUP-6b (20 mg, 59 μmol) with HOBt, Boc-L1-2b (31 mg, ESI m/z 964 (M+H)⁺) was obtained. Boc-L1-2b was dissolved in DCM (4 mL). To the solution was added TFA (0.5 mL) and the reaction mixture was stirred at room temperature for half an hour until Boc was totally removed according to LCMS. The volatiles were removed in vacuo to give compound L1-2b. ESI m/z 433 (M/2+H)⁺.

### (4S)-4-Amino-5-(4-{[({4-[(2S)-5-(carbamoylamino)-2-[(2S)-2-[(2S)-4-carboxy-2-{[(9H-fluoren-9-ylmethoxy)carbonyl]amino}butanamido]-3-methylbutanamido]pentanamido]phenyl}methoxy)carbonyl]amino}phenyl)-2,2-dimethylpentanoic acid (L1-2c)

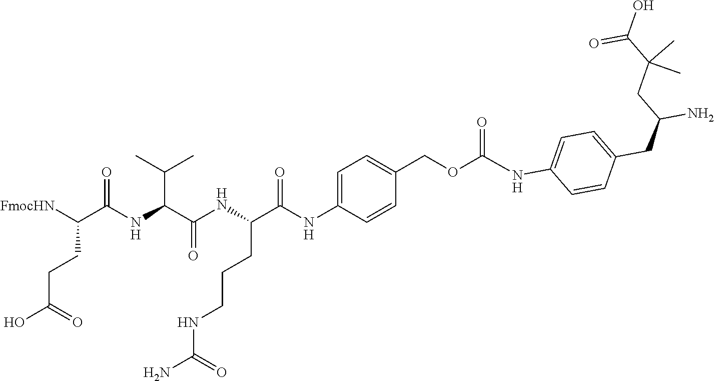

Following General Procedure XII using Fmoc-Glu(O'Bu)-Val-Cit-PABC-PNP (L1-1c) (0.10 g, 0.11 mmol) and amine TUP-6b with HOBt, Boc-L1-2c (ESI m/z: 1151 (M+H)⁺) was obtained. Boc-L1-2c was dissolved in DCM (5 mL). To the solution was added TFA (1 mL) and the reaction mixture was stirred at room temperature for an hour, and monitored by LCMS. The resulting mixture was concentrated in vacuo and the residue was purified by reverse phase flash chromatography (0-100% acetonitrile in aq. TFA (0.01%)) to give L1-2c. ESI m/z: 994 (M+H)⁺.

Synthesis of LP1 and LP2 was consistent with FIG. 13.

Synthesis of intermediate LP4-7 was consistent with the scheme below.

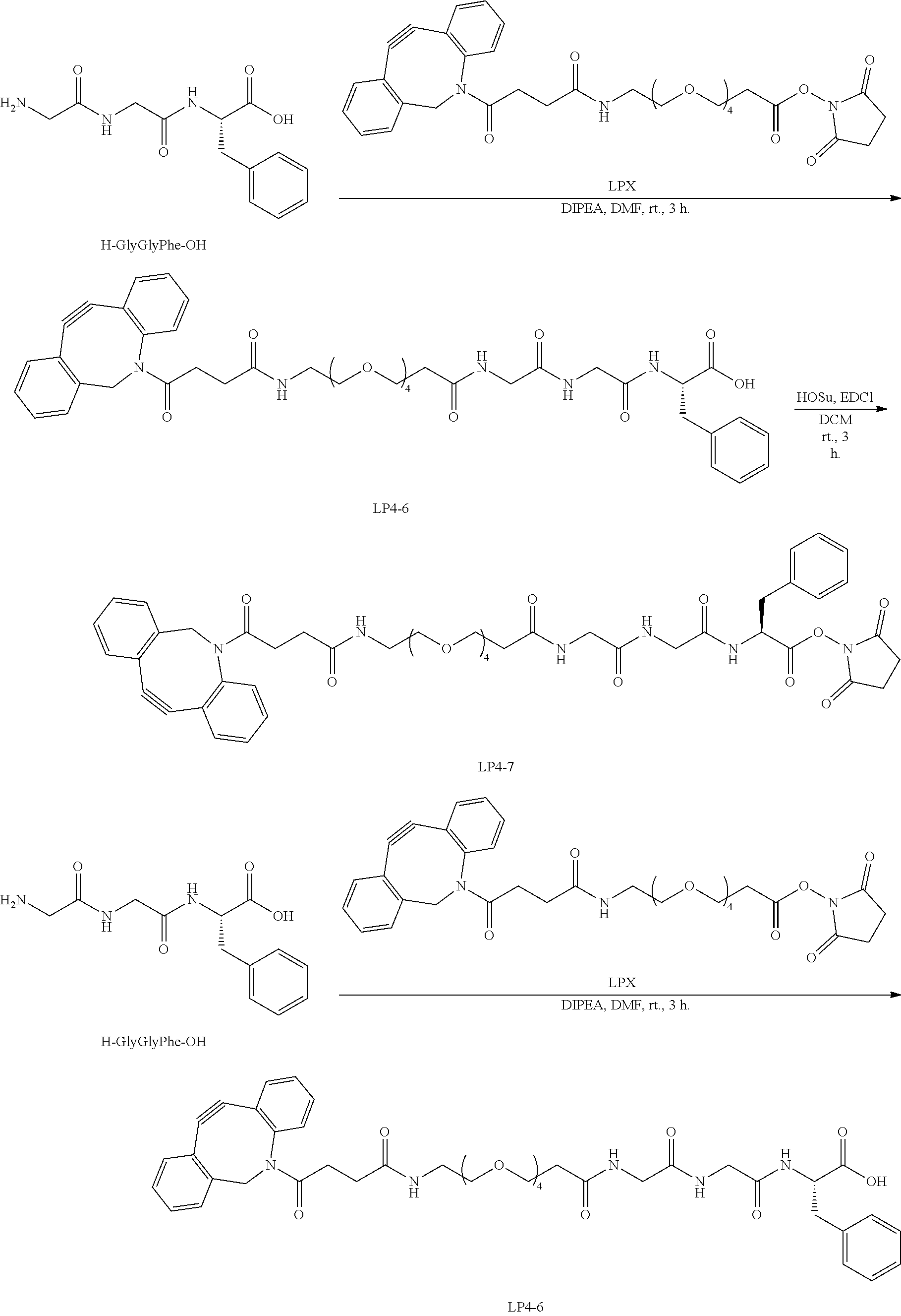

Following General Procedure XI from amine H-GlyGlyPhe-OH with OSu ester (LPX), LP4-6 was obtained.

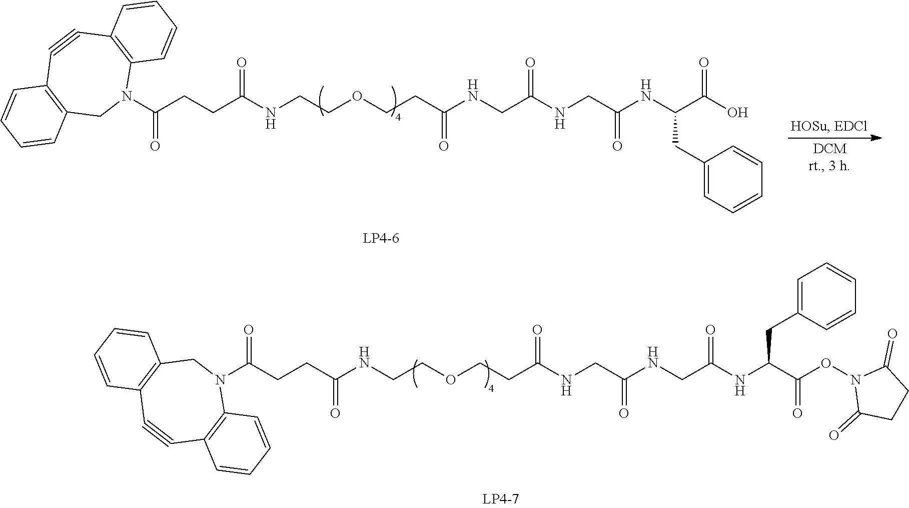

Following General Procedure XI from amine LP4-6 with OSu ester (LPX), LP4-7 was obtained.

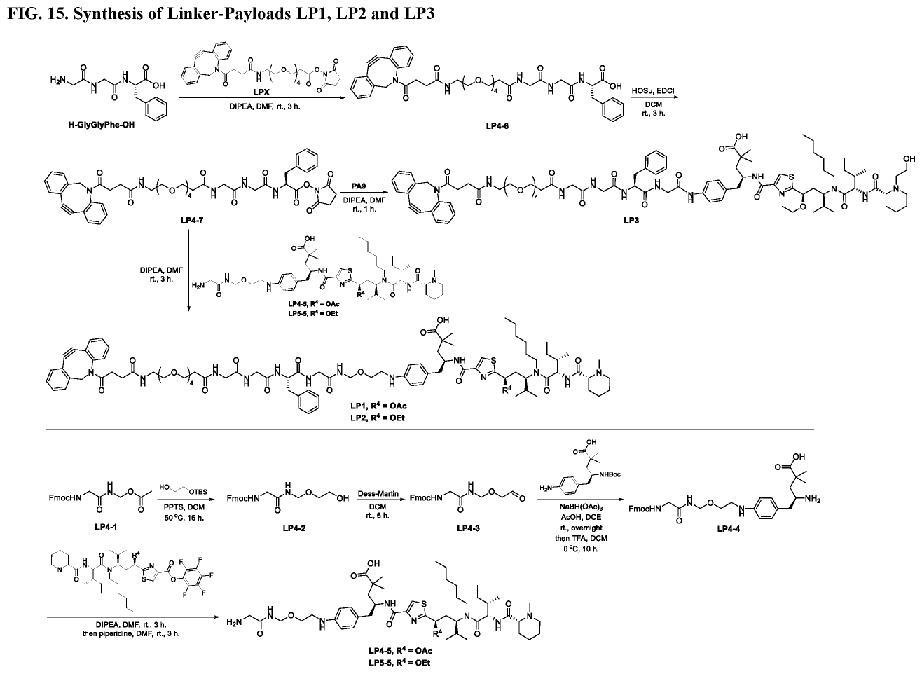

Synthesis of LP1-LP3 was consistent with FIG. 15.

Synthesis of LP1 and LP2 was consistent with the scheme below.

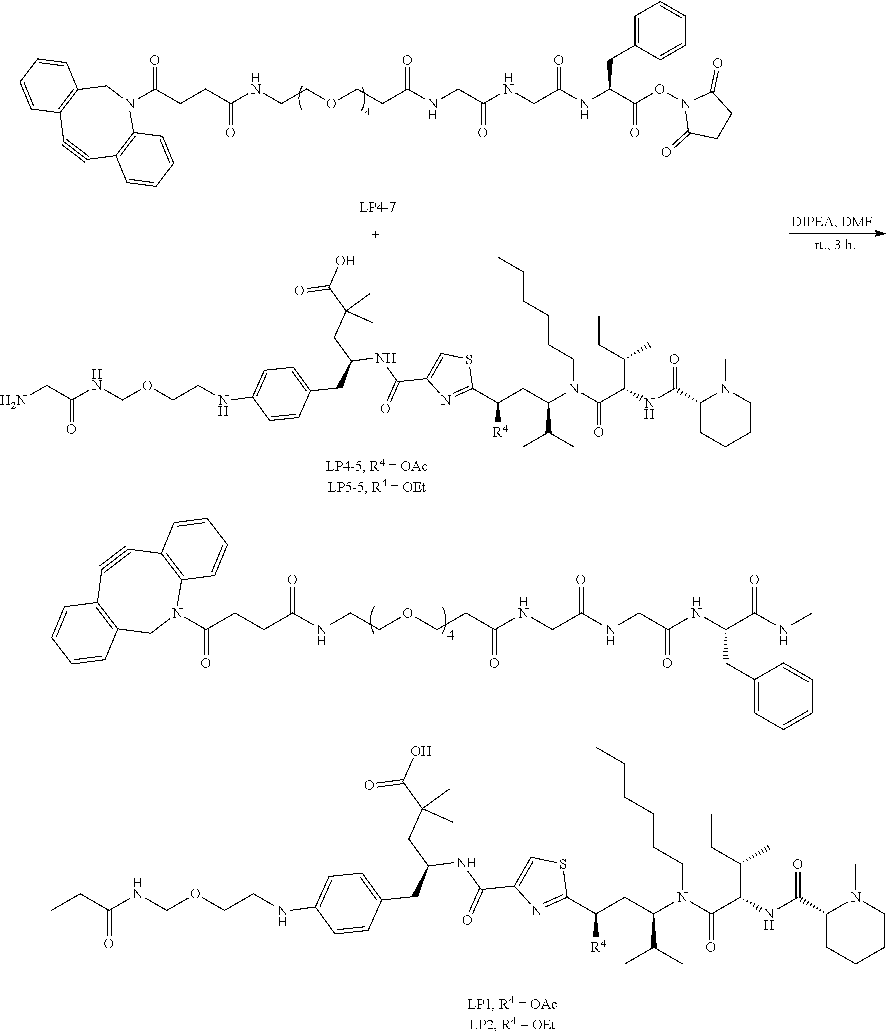

Following General Procedure XI from amine (LP4-5 or LP5-5) with -OSu ester (LP4-7), linker-payloads LP1 and LP2 were obtained.

Synthesis of LP3 was consistent with the scheme below.

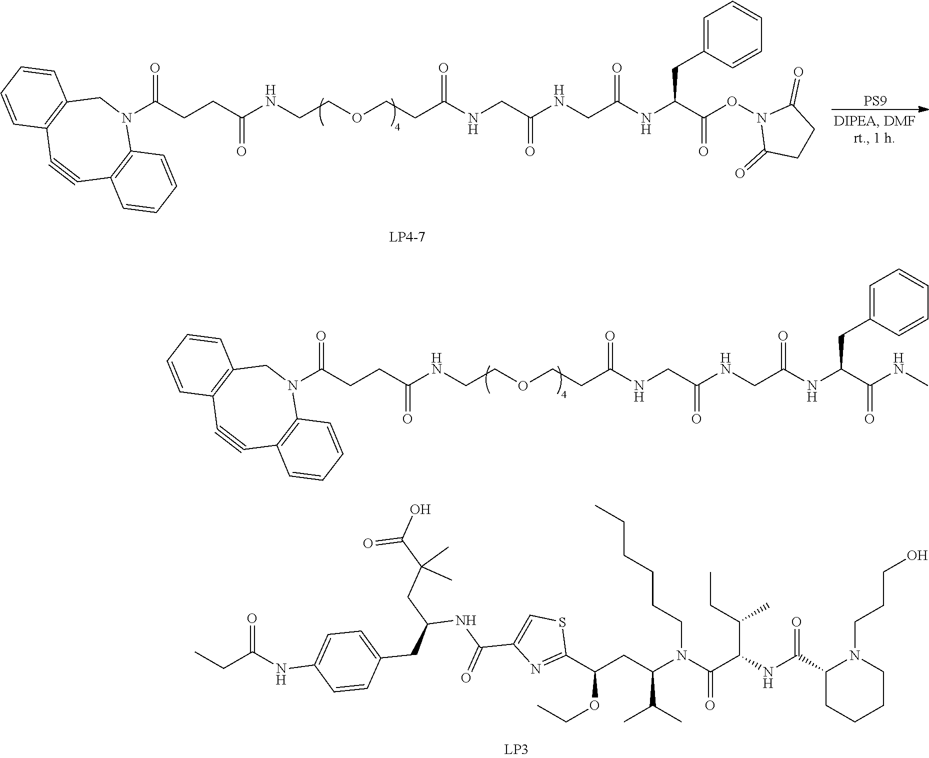

Following General Procedure XI from amine PA9 with -OSu ester (LP4-7), linker-payload LP3 was obtained.

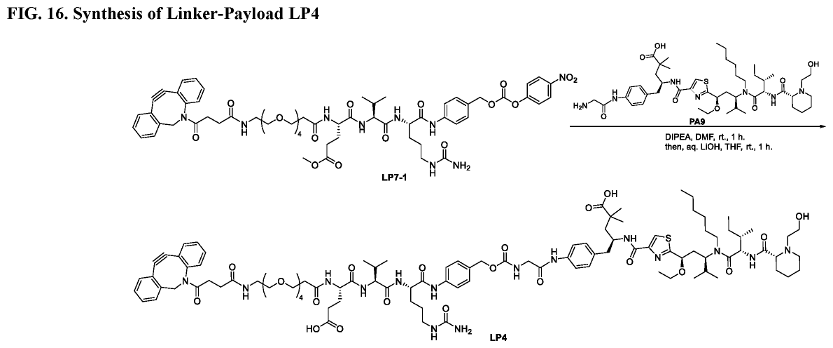

Synthesis of LP4 was consistent with FIG. 16.

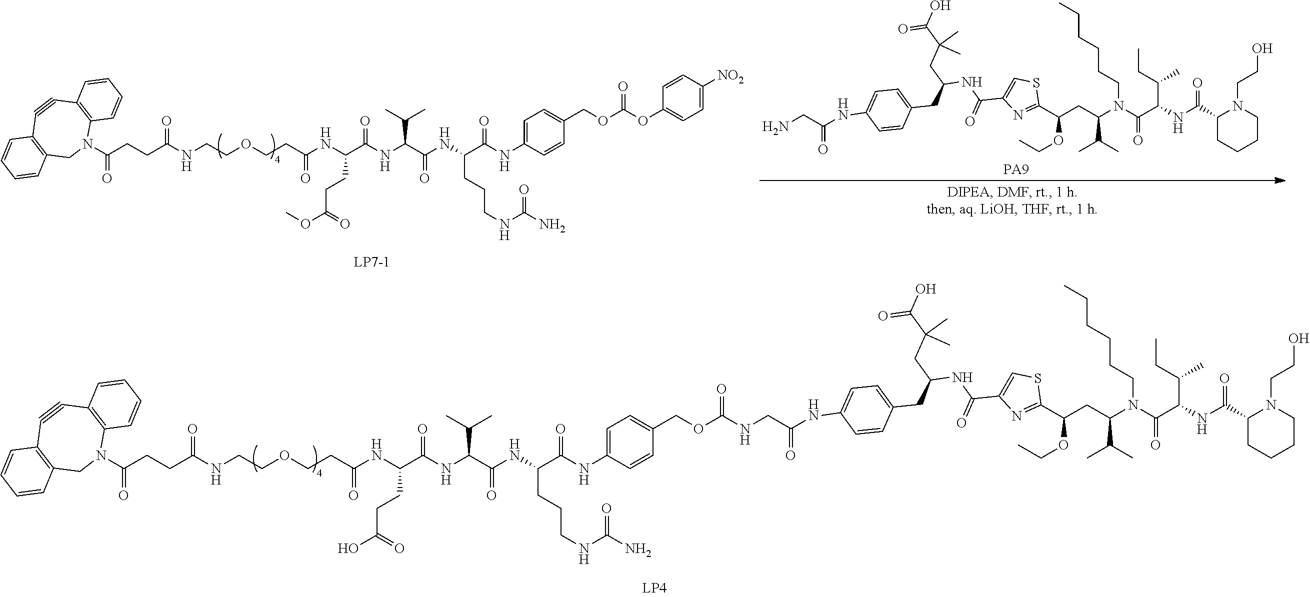

Linker-payload LP4 was prepared from intermediate LP7-1 and amine (PA9) according to General Procedure XII and was consistent with the scheme above.

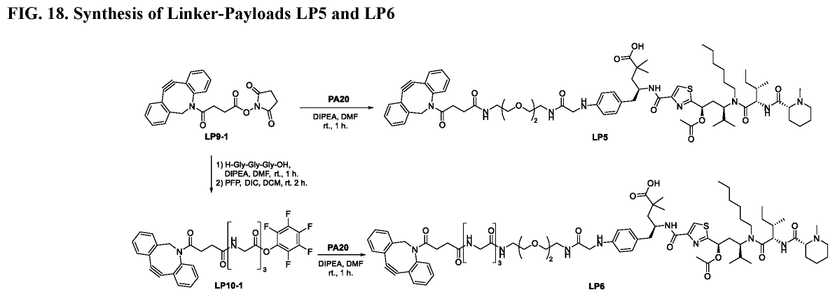

Synthesis of LP5 and LP6 was consistent with FIG. 18.

Synthesis of LP5 was consistent with the scheme below.

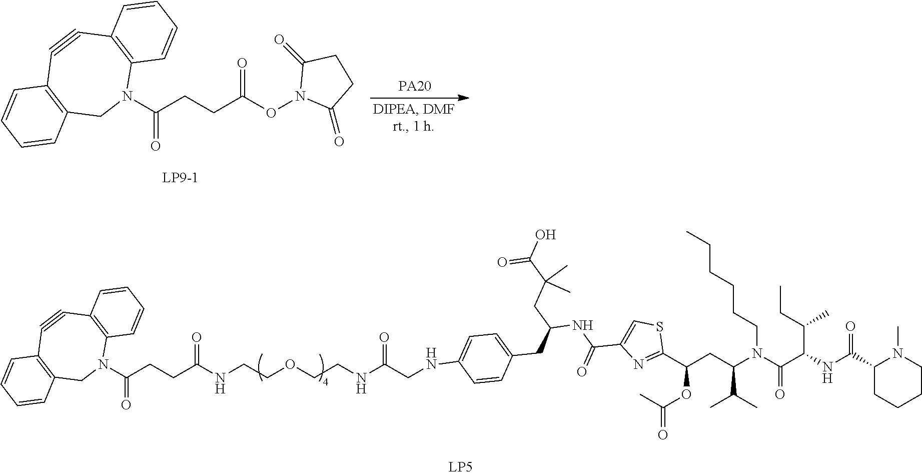

Following General Procedure XI from amine (PA20) with -OSu ester (LP9-1), linker-payload LP5 was obtained.

### Synthesis of LP6

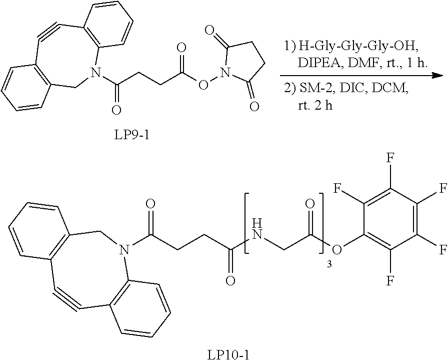

In two steps, using General Procedure XI, amine (H-Gly-Gly-Gly-OH) with -OSu ester (LP9-1) furnished an acid intermediate which was activated using General Procedure VI to give an ester (LP10-1).

Synthesis of LP6 was consistent with the scheme below.

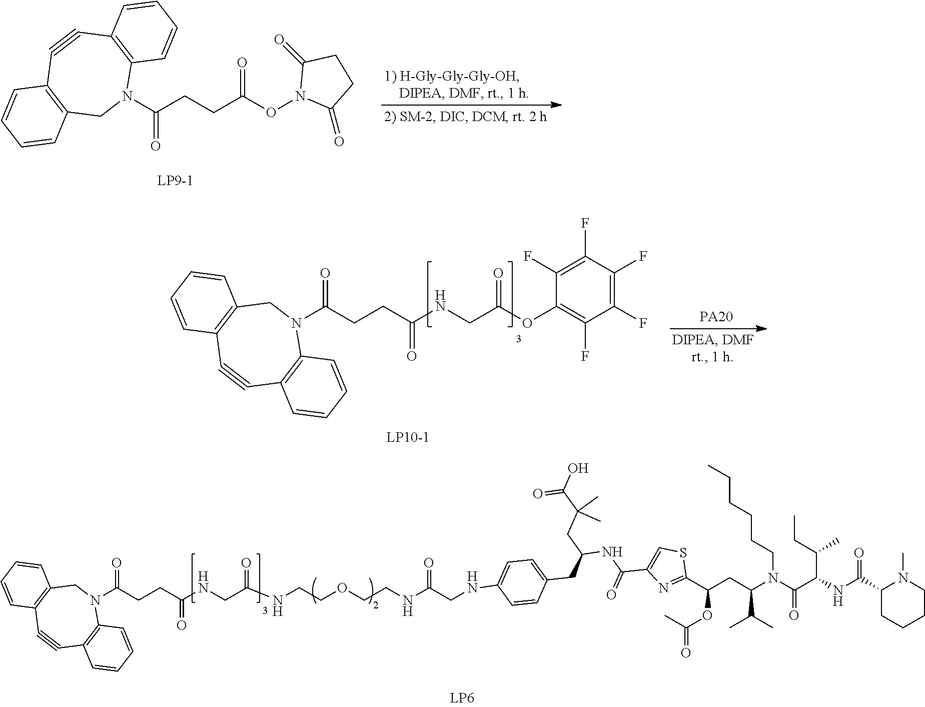

Following General Procedure XI from treating amine PA20 with ester (LP10-1), linker-payload LP6 was obtained.

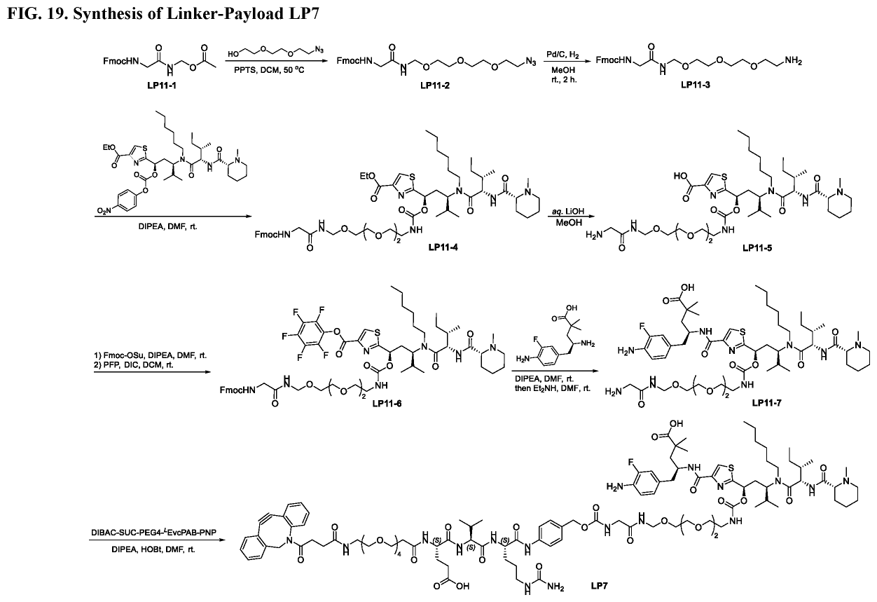

Synthesis of LP7 was consistent with FIG. 19.

Synthesis of LP11-4 was consistent with the scheme below.

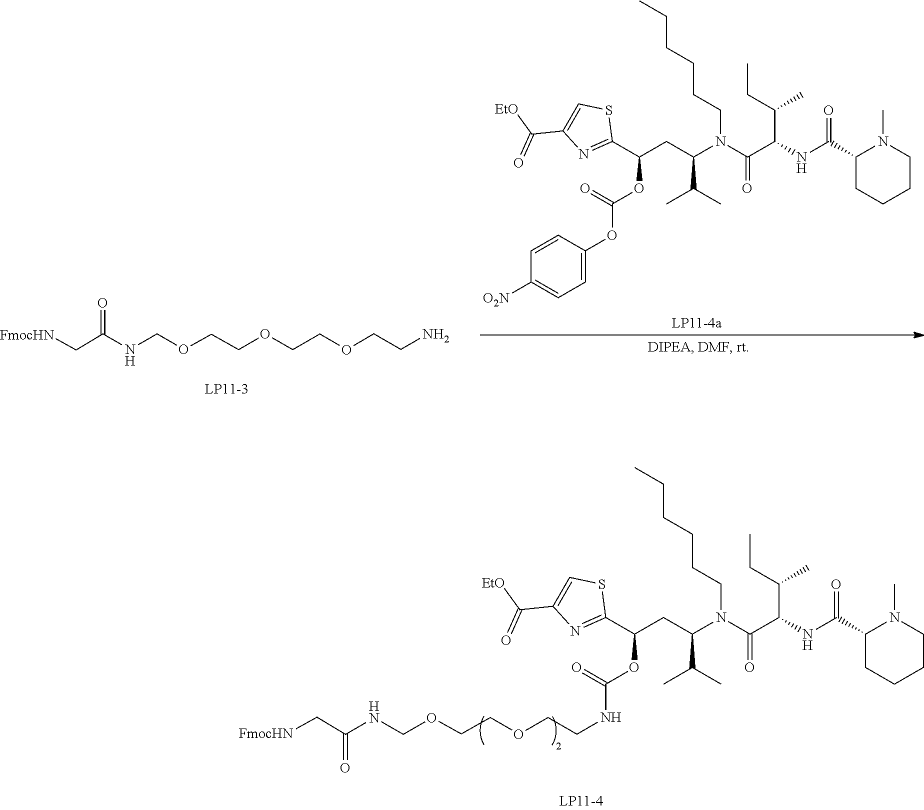

LP11-4 was prepared from intermediate LP11-4a and amine (LP11-3) according to General Procedure XII as shown in the Scheme above.

Synthesis of LP11-5 was consistent with the scheme below.

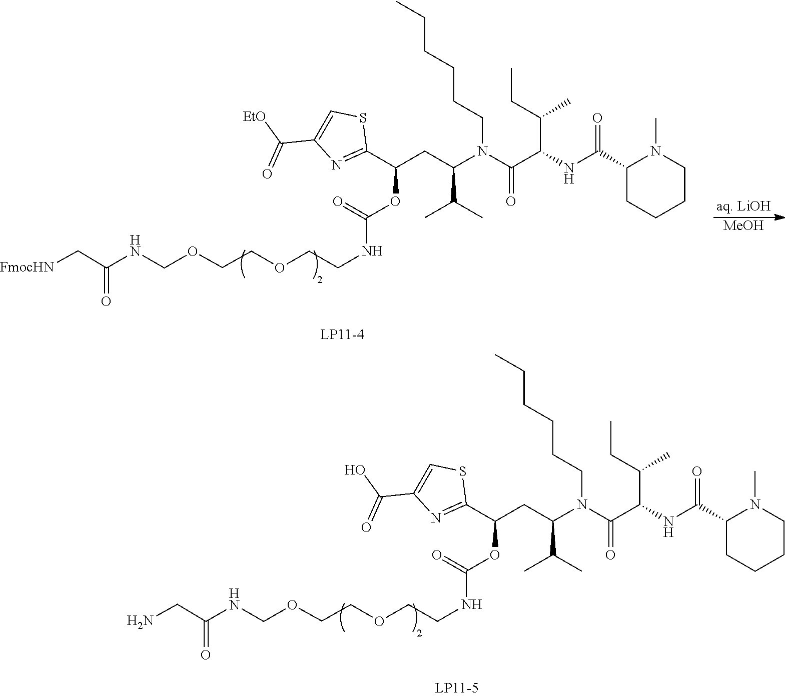

LP11-5 was prepared from intermediate LP11-4 according to General Procedure IV as shown in the scheme above.

Synthesis of LP11-6 was consistent with the scheme below.

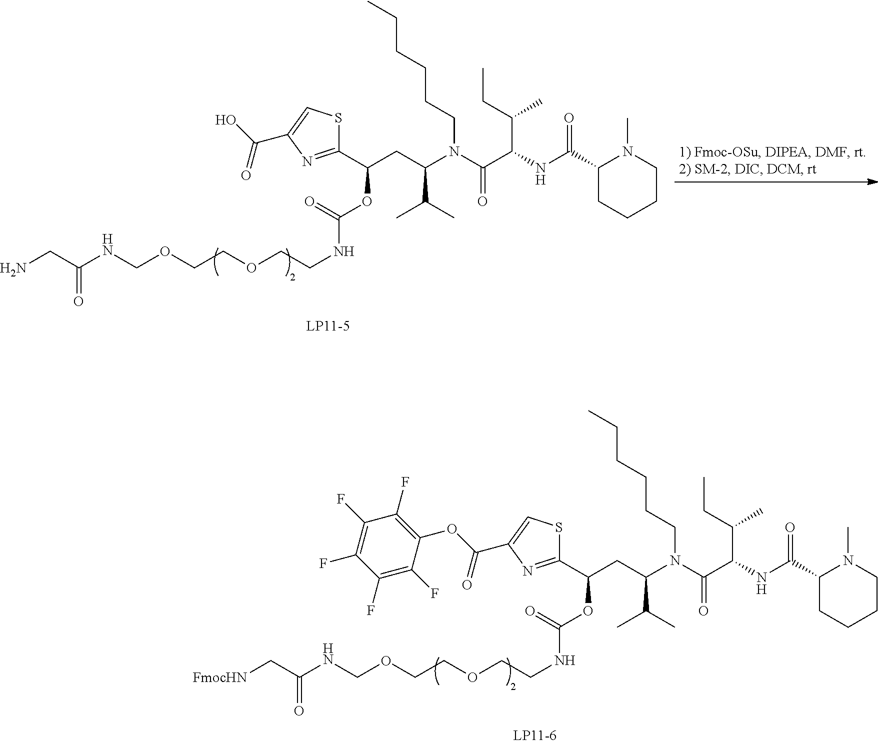

In two steps using General Procedure XI, amine LP11-5 with -OSu ester, furnished an acid intermediate which was activated using General Procedure VI to give an ester (LP11-6) as shown in the scheme above.

Synthesis of LP11-7 was consistent with the scheme below.

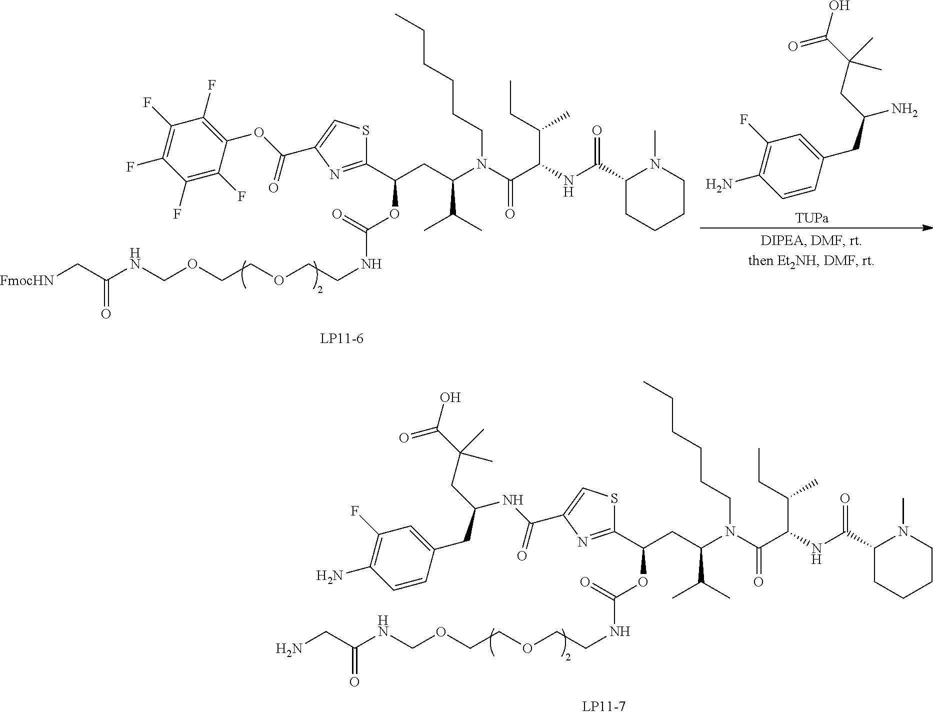

Synthesis of LP7 was consistent with the scheme below

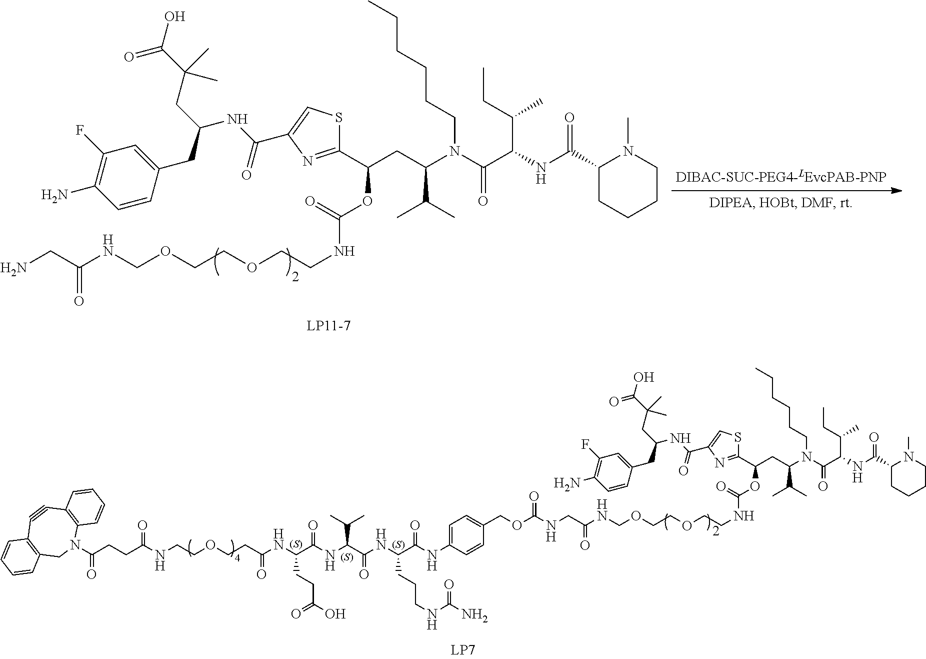

Following General Procedure XII from treating amine LP11-7 with PNP ester (DIBAC-SUC-PEG4-LEvcPABC-PNP), linker-payload LP7 was obtained as shown in the scheme above.

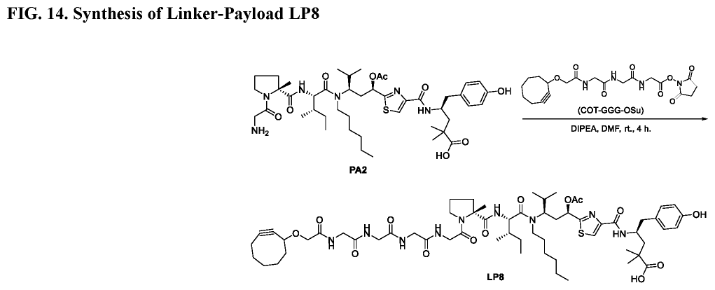

Synthesis of LP8 was consistent with FIG. 14.

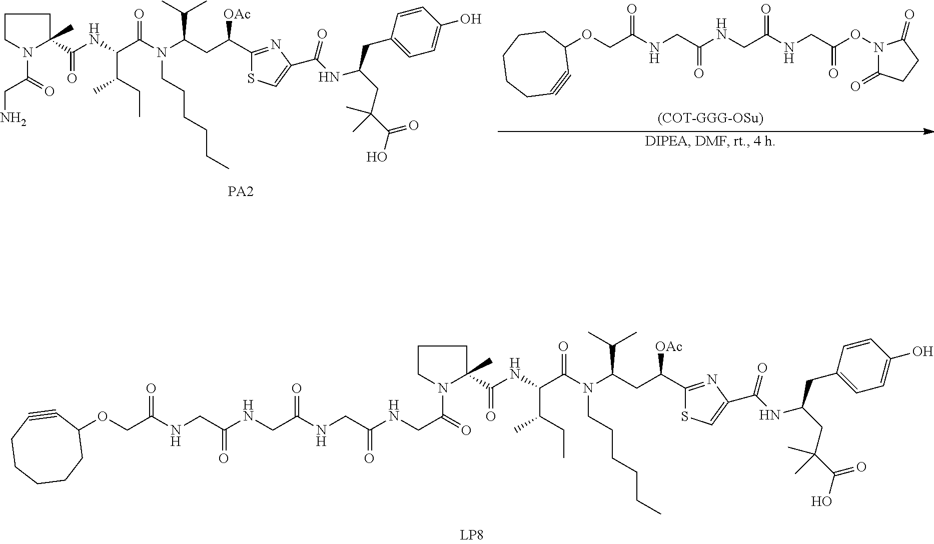 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(O)cc2)CC(C)(C)C(=O)O)cs1)C(C)C ; O=C(CNC(=O)COC1C#CCCCCC1)NCC(=O)NCC(=O)ON1C(=O)CCC1=O ; CCCCCCN(C(=O)[C@@H](NC(=O)[C@@]1(C)CCCN1C(=O)CNC(=O)CNC(=O)CNC(=O)CNC(=O)COC1C#CCCCCC1)[C@@H](C)CC)[C@H](C[C@@H](OC(C)=O)c1nc(C(=O)N[C@@H](Cc2ccc(O)cc2)CC(C)(C)C(=O)O)cs1)C(C)C]

Following General Procedure XI from amine PA2 with -OSu ester (COT-GGG-OSu), linker-payload LP8 was obtained as shown in the scheme above.

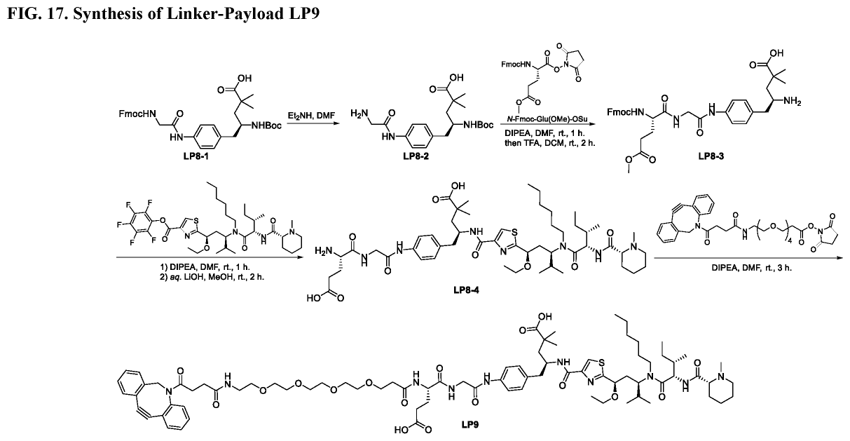

Synthesis of LP9 was consistent with FIG. 17.

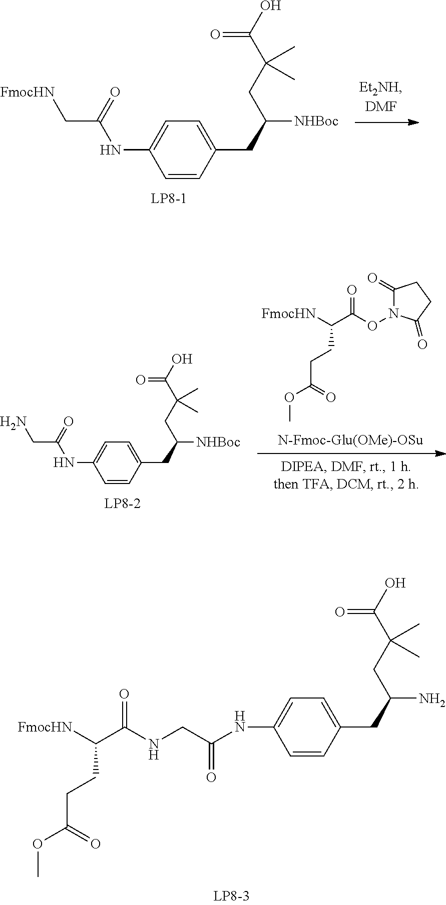

Linker-payload LP4 was prepared from intermediate LP7-1 and amine (PA9) according to General Procedure XI as shown in the scheme above.

Synthesis of intermediate LP8-4 was consistent with the scheme below.

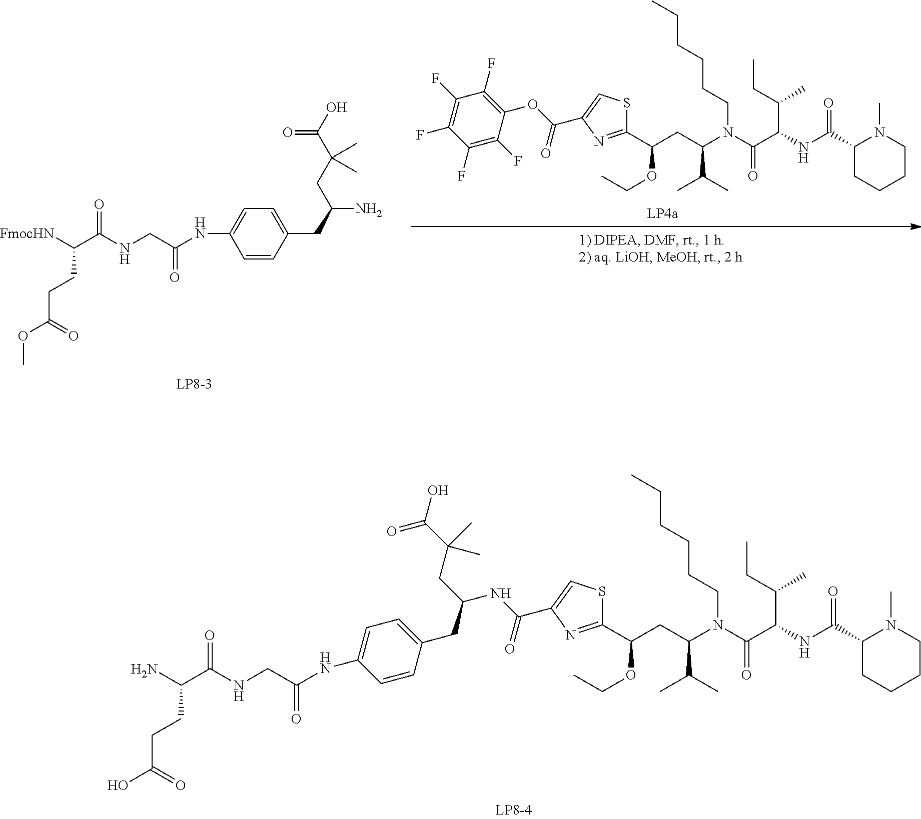

LP8-4 was prepared from intermediate amine (LP8-3) and ester (LP4a) according to General Procedure VII as shown in the scheme above.

Synthesis of Linker-payload LP9 was consistent with the scheme below.

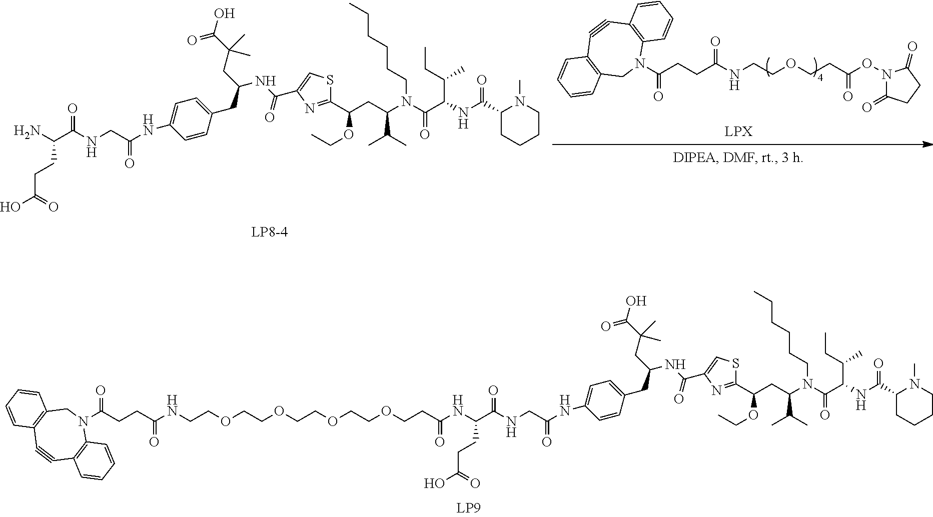

Following General Procedure XI from amine LP8-4 with -OSu ester (LPX), linker-payload LP9 was obtained as shown in the scheme above.

Synthesis of LP10 and LP11 was consistent with FIG. 13.

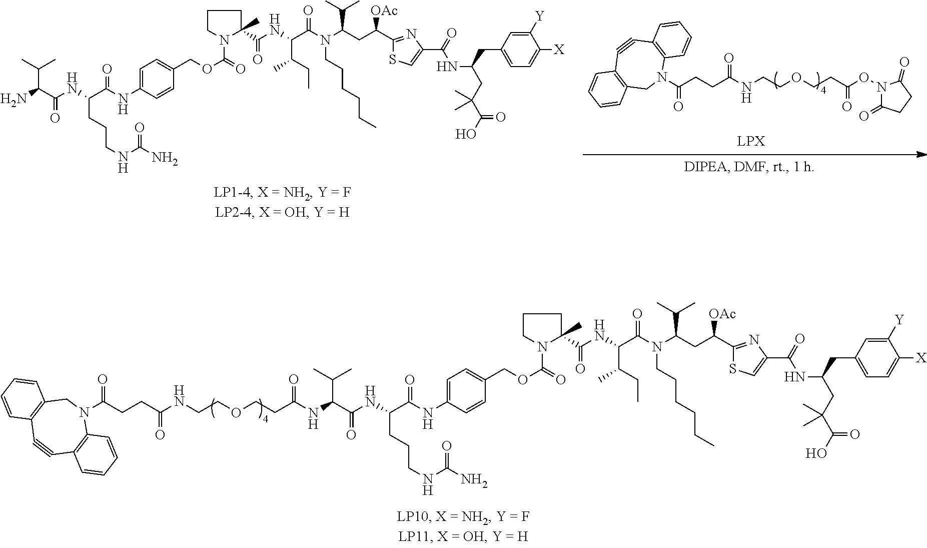

Following General Procedure XI from amine LP1-4 and LP2-4 with -OSu ester (LPX), linker-payloads LP10 and LP11 were obtained as shown in the scheme above.

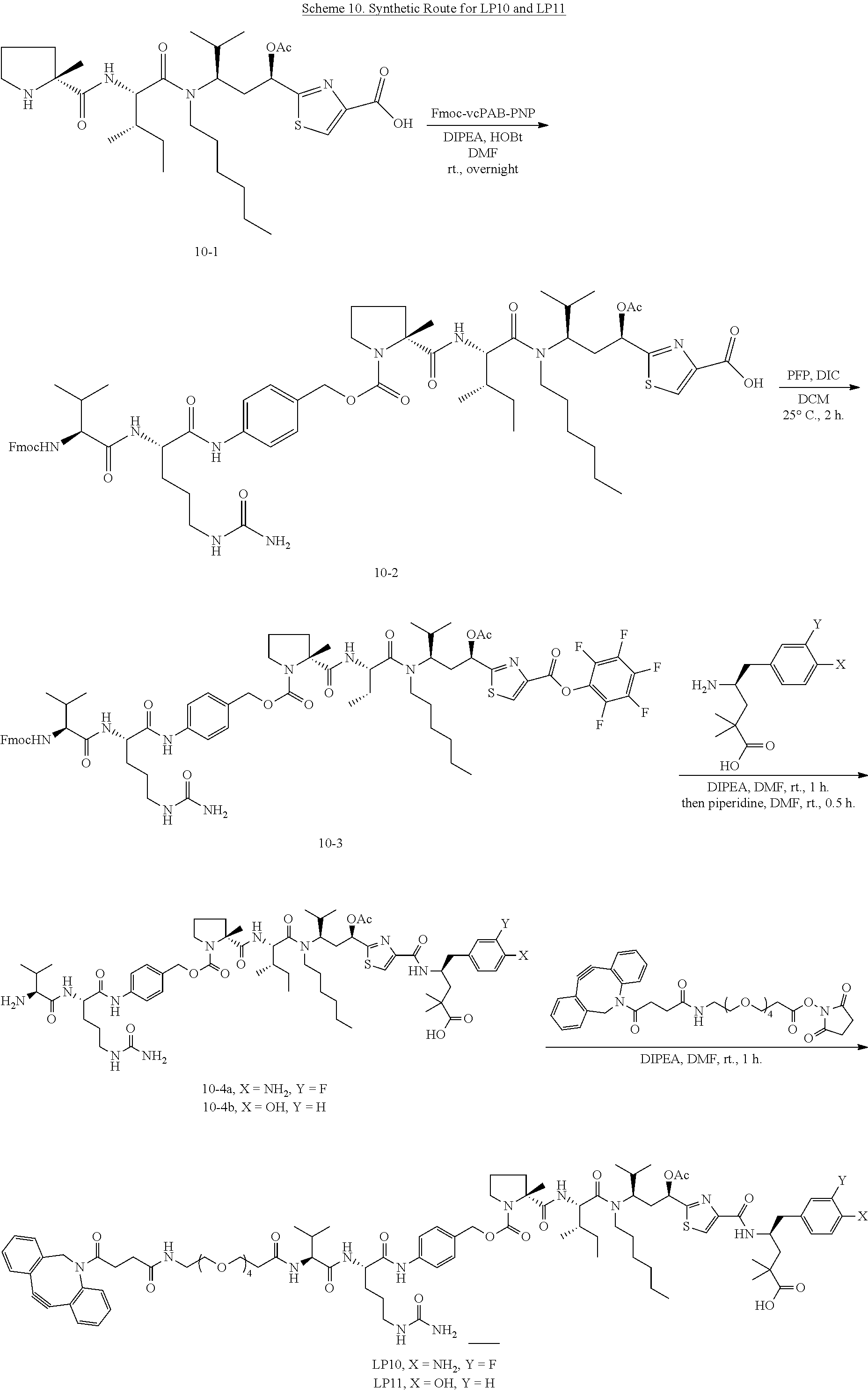

### LP10

### 2-((1R,3R)-3-((2S,3S)-2-((R)-1-(((4-((S)-2-((S)-2-((((9H-fluoren-9-yl)methoxy)carbonyl)amino)-3-methylbutanamido)-5-ureidopentanamido)benzyl)oxy)carbonyl)-2-methylpyrrolidine-2-carboxamido)-N-hexyl-3-methylpentanamido)-1-acetoxy-4-methylpentyl)thiazole-4-carboxylic acid (10-2)

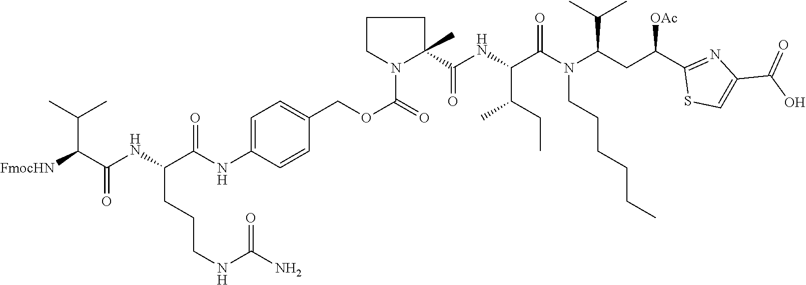

To a mixture of compound 10-1 (0.20 g, 0.34 mmol) in DMF (10 mL) was successively added Fmoc-vcPAB-PNP (1.0 g, 1.3 mmol), HOBt (92 mg, 0.68 mmol), and DIPEA (0.18 g, 1.4 mmol), and the reaction mixture was stirred at room temperature for eighteen hours, which was monitored by LCMS. The reaction mixture was directly purified by reversed phase flash chromatography (30% acetonitrile in aq. TFA (0.01%)) to give compound 10-2 (0.12 g, 29% yield) as a pale yellow oil. ESI m/z 611.8 (M/2+H)⁺.

### (S)-4-(2-((1R,3R)-1-acetoxy-3-((2S,3S)-2-((R)-1-(((4-((S)-2-((S)-2-amino-3-methylbutanamido)-5-ureidopentanamido)benzyl)oxy)carbonyl)-2-methylpyrrolidine-2-carboxamido)-N-hexyl-3-methylpentanamido)-4-methylpentyl)thiazole-4-carboxamido)-5-(4-amino-3-fluorophenyl)-2,2-dimethylpentanoic acid (10-4a)

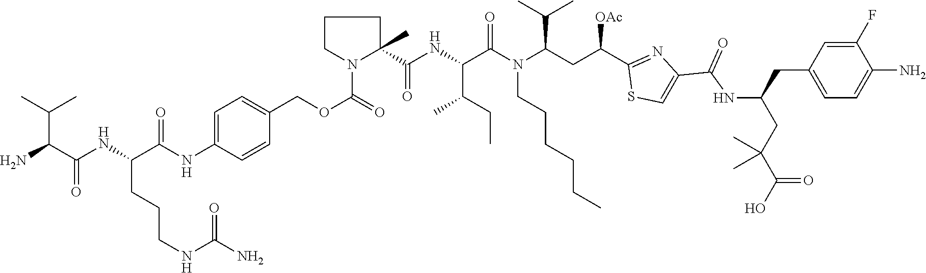 [SMILES: CCCCCCN(C(=O)[C@@H](NC(=O)[C@@]1(C)CCCN1C(=O)OCc1ccc(NC(=O)[C@H](CCCNC(N)=O)NC(=O)[C@@H](N)C(C)C)cc1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C]

Following Alternative General Procedure V starting from compound 10-2 (60 mg, 49 μmol), compound 10-3 (68 mg, crude) was obtained as a light yellow oil. ESI m/z 695.3 (M/2+H)⁺. Following Alternative General Procedure VI (then Fmoc removal) using crude compound 10-3 obtained above and treating with TupA, compound 10-4a (12 mg, 20% yield from 10-2) was obtained as a white solid. ESI m/z 619.0 (M/2+H)⁺.

### (4S)-4-({2-[(1R,3R)-1-(acetyloxy)-3-[(2S,3S)-2-{[(2R)-1-[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]-2-methylpyrrolidin-2-yl]formamido}-N-hexyl-3-methylpentanamido]-4-methylpentyl]-1,3-thiazol-4-yl}formamido)-5-(4-amino-3-fluorophenyl)-2,2-dimethylpentanoic acid (LP10)

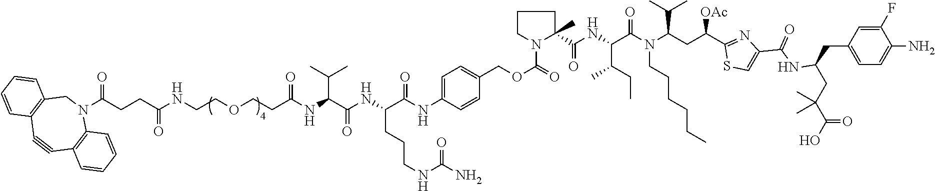

To a solution of DIBAC-PEG4-OSu (10 mg, 15 μmol) in DMF (5 mL) was added DIPEA (4.0 mg, 29 μmol) and compound 10-4a (12 mg, 9.7 μmol) and the reaction mixture was stirred at room temperature for an hour, which was monitored by LCMS. The resulting mixture was directly separated by prep-HPLC (5-90% acetonitrile in aq. TFA (0.01% TFA)) to give LP10 (2.9 mg, 17% yield) as a white solid. ESI m/z 886.0 (M/2+H)⁺. ¹H NMR (500 MHz, DMSO-d₆) δ 10.01 (s, 1H), 8.16 (s, 1H), 7.90-7.88 (m, 1H), 7.78 (t, J=5.2 Hz, 1H), 6.69-7.67 (m, 1H), 7.63-7.56 (m, 4H), 7.52-7.44 (m, 3H), 7.40-7.20 (m, 5H), 6.78-6.72 (m, 1H), 6.68-6.59 (m, 2H), 6.05-5.96 (m, 1H), 5.69-5.61 (m, 1H), 5.45-5.41 (m, 2H), 5.04-4.99 (m, 1H), 4.96-4.90 (m, 3H), 4.49-4.43 (m, 1H), 4.40-4.35 (m, 1H), 4.25-4.19 (m, 2H), 3.65-3.52 (m, 6H), 3.50-3.42 (m, 15H), 3.33-3.28 (m, 3H), 3.11-3.05 (m, 2H), 3.00-2.93 (m, 2H), 2.62-2.54 (m, 3H), 2.47-2.43 (m, 1H), 2.38-2.32 (m, 1H), 2.27-2.20 (m, 2H), 2.15-2.11 (m, 3H), 2.04-1.94 (m, 3H), 1.86-1.82 (m, 1H), 1.79-1.66 (m, 6H), 1.64-1.54 (m, 4H), 1.50-1.40 (m, 5H), 1.35-1.20 (m, 9H), 1.06 (s, 6H), 0.96-0.90 (m, 3H), 0.87-0.74 (m, 17H), 0.70-0.64 (m, 2H), 0.57-0.50 (m, 1H) ppm. ¹⁹F NMR (376 MHz, DMSO-d₆) −135.46 ppm.

### LP11

### (S)-4-(2-((1R,3R)-1-acetoxy-3-((2S,3S)-2-((R)-1-(((4-((S)-2-((S)-2-amino-3-methylbutanamido)-5-ureidopentanamido)benzyl)oxy)carbonyl)-2-methylpyrrolidine-2-carboxamido)-N-hexyl-3-methylpentanamido)-4-methylpentyl)thiazole-4-carboxamido)-5-(4-hydroxyphenyl)-2,2-dimethylpentanoic acid (10-4b)

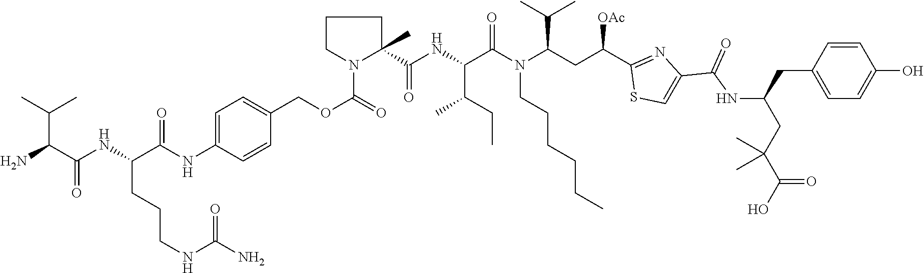 [SMILES: CCCCCCN(C(=O)[C@@H](NC(=O)[C@@]1(C)CCCN1C(=O)OCc1ccc(NC(=O)[C@H](CCCNC(N)=O)NC(=O)[C@@H](N)C(C)C)cc1)[C@@H](C)CC)[C@H](C[C@@H](OC(C)=O)c1nc(C(=O)N[C@@H](Cc2ccc(O)cc2)CC(C)(C)C(=O)O)cs1)C(C)C]

Following Alternative General Procedure V starting from compound 10-2 (60 mg, 49 μmol), compound 10-3 (68 mg, crude) was obtained as a light yellow oil. ESI m/z 695.0 (M/2+H)⁺.

Following Alternative General Procedure VI (then Fmoc removal) using crude compound 10-3 obtained above and treating with TupB, compound 10-4b (12 mg, 20% yield from was obtained as a white solid. ESI m/z 610.3 (M/2+H)⁺.

### (4S)-4-({2-[(1R,3R)-1-(acetyloxy)-3-[(2S,3S)-2-{[(2R)-1-[({4-[(2S)-2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]-2-methylpyrrolidin-2-yl]formamido}-N-hexyl-3-methylpentanamido]-4-methylpentyl]-1,3-thiazol-4-yl}formamido)-5-(4-hydroxyphenyl)-2,2-dimethylpentanoic acid (LP11)

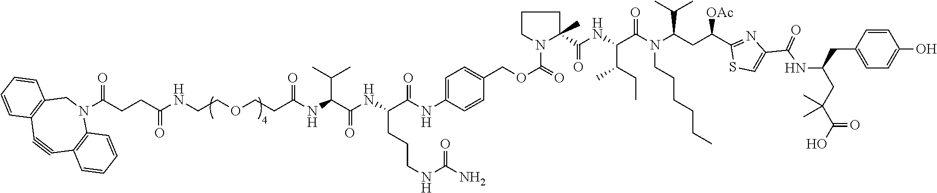

Following a similar procedure as LP10 except using 10-4b (12 mg, 9.8 μmol) instead of 10-4a, linker-payload LP11 (2.3 mg, 13% yield) was obtained as a white solid. ESI m/z 877.5 (M/2+H)⁺. ¹H NMR (400 MHz, DMSO-d₆) δ 10.05 (s, 1H), 9.22 (s, 1H), 8.18 (s, 1H), 7.93-7.91 (m, 1H), 7.80 (t, J=5.6 Hz, 1H), 7.70-7.68 (m, 1H), 7.63-7.59 (m, 4H), 7.52-7.45 (m, 3H), 7.38-7.29 (m, 4H), 7.22-7.20 (m, 1H), 6.95-6.93 (m, 2H), 6.64-6.62 (m, 2H), 5.66-5.63 (m, 1H), 5.45-5.43 (m, 2H), 5.04-4.94 (m, 2H), 4.48-4.35 (m, 3H), 4.23-4.18 (m, 2H), 3.63-3.59 (m, 2H), 3.57-3.51 (m, 3H), 3.46-3.44 (m, 16H), 3.30-3.25 (m, 3H), 3.13-3.07 (m, 2H), 3.02-2.91 (m, 3H), 2.71-2.67 (m, 1H), 2.60-2.54 (m, 2H), 2.41-2.37 (m, 2H), 2.27-2.22 (m, 2H), 2.14-2.06 (m, 4H), 2.03-1.92 (m, 4H), 1.79-1.69 (m, 4H), 1.68-1.61 (m, 4H), 1.54-1.45 (m, 5H), 1.23-1.17 (m, 8H), 1.03 (s, 6H), 0.96-0.94 (m, 2H), 0.89-0.75 (m, 19H), 0.66-0.61 (m, 2H), 0.52-0.49 (m, 1H) ppm.

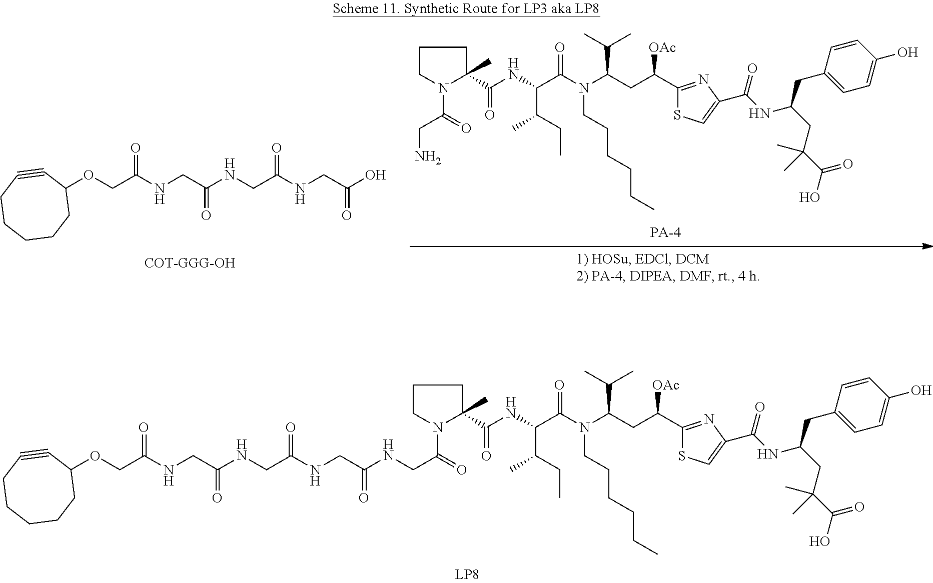 [SMILES: O=C(O)CNC(=O)CNC(=O)CNC(=O)COC1C#CCCCCC1 ; CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(O)cc2)CC(C)(C)C(=O)O)cs1)C(C)C ; CCCCCCN(C(=O)[C@@H](NC(=O)[C@@]1(C)CCCN1C(=O)CNC(=O)CNC(=O)CNC(=O)CNC(=O)COC1C#CCCCCC1)[C@@H](C)CC)[C@H](C[C@@H](OC(C)=O)c1nc(C(=O)N[C@@H](Cc2ccc(O)cc2)CC(C)(C)C(=O)O)cs1)C(C)C]

### LP8

### (4S)-4-({2-[(1R,3R)-1-(acetyloxy)-3-[(2S,3S)-2-{[(2R)-1-{2-[2-(2-{2-12-(cyclooct-2-yn-1-yloxy)acetamido]acetamido}acetamido)acetamido]acetyl}-2-methylpyrrolidin-2-yl]formamido}-N-hexyl-3-methylpentanamido]-4-methylpentyl]-1,3-thiazol-4-yl}formamido)-5-(4-hydroxyphenyl)-2,2-dimethylpentanoic acid (LP8)

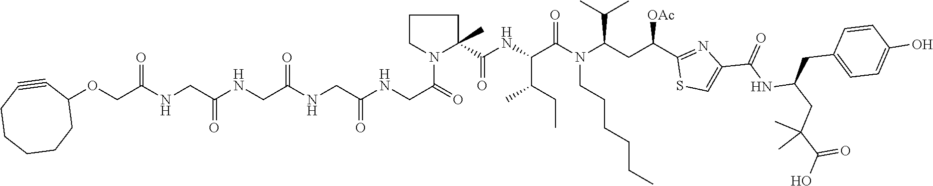 [SMILES: CCCCCCN(C(=O)[C@@H](NC(=O)[C@@]1(C)CCCN1C(=O)CNC(=O)CNC(=O)CNC(=O)CNC(=O)COC1C#CCCCCC1)[C@@H](C)CC)[C@H](C[C@@H](OC(C)=O)c1nc(C(=O)N[C@@H](Cc2ccc(O)cc2)CC(C)(C)C(=O)O)cs1)C(C)C]

To a solution of COT-GGG-OH (50 mg, 0.28 mmol) in DMF (3 mL) was added HOSu (33 mg, 0.28 mmol) and EDCI (54 mg, 0.28 mmol) and the reaction mixture was then stirred at room temperature for four hours, which was monitored by LCMS. The resulting mixture was quenched with water (20 mL) and the mixture was extracted with DCM (3×20 mL). The combined organic solution was washed with brine (20 mL), dried over anhydrous sodium sulfate, filtered, and concentrated in vacuo to give COT-GGG-OSu (50 mg, crude, ESI m/z 451.3 (M+H)⁺) as a white solid, which was used directly without further purification. To a solution of payload PA-4 aka PA2 (6.0 mg, 6.9 μmol) in DMF (3 mL) was added COT-GGG-OSu (16 mg) obtained above and DIPEA (4.0 mg, 31 μmol) and the reaction mixture was stirred at room temperature for four hours, which was monitored by LCMS. The resulting mixture was directly separated by prep-HPLC (5-95% acetonitrile in aq. ammonium bicarbonate (10 mM)) to give linker-payload LP8 (1.0 mg, 12% yield) as a white solid. ESI m/z 604.0 (M/2+H)⁺. ¹H NMR (400 MHz, DMSO-d₆) δ 9.87 (s, 1H), 9.21 (s, 1H), 8.21-8.17 (m, 1H), 8.16-8.14 (m, 2H), 7.83-7.81 (m, 2H), 6.93 (d, J=8.4 Hz, 2H), 6.62 (d, J=8.4 Hz, 2H), 5.62 (d, J=3.6 Hz, 1H), 4.44 (t, J=8.0 Hz, 1H), 4.34-4.30 (m, 1H), 3.96-3.90 (m, 2H), 3.83-3.82 (m, 1H), 3.79-3.73 (m, 7H), 2.69-2.65 (m, 3H), 2.35-2.31 (m, 4H), 2.14 (br s, 4H), 2.10-2.03 (m, 6H), 1.95-1.90 (m, 3H), 1.81-1.78 (m, 2H), 1.70-1.64 (m, 3H), 1.60-1.55 (m, 3H), 1.50-1.40 (m, 4H), 1.30-1.23 (m, 8H), 1.92-1.89 (m, 1H), 1.09-1.03 (m, 7H), 0.95 (d, J=4.0 Hz, 3H), 0.88-0.83 (m, 4H), 0.79-0.73 (m, 7H), 0.69-0.64 (m, 3H) ppm.

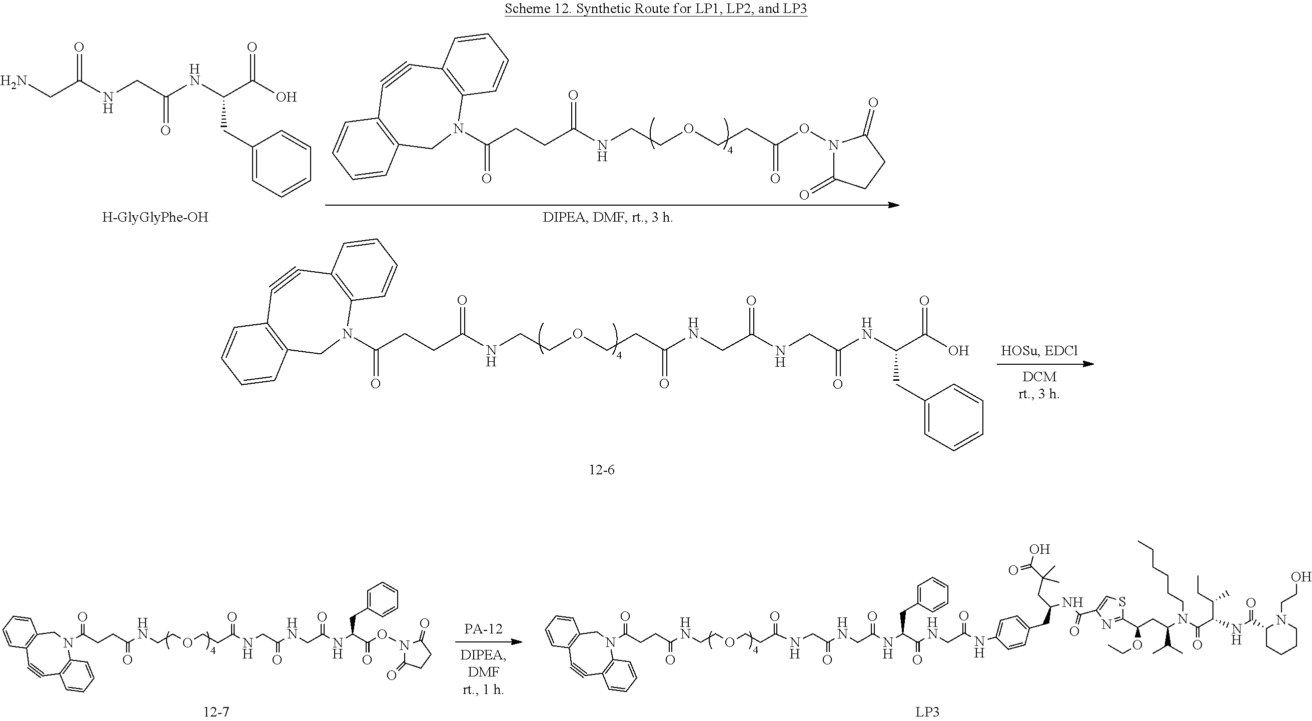

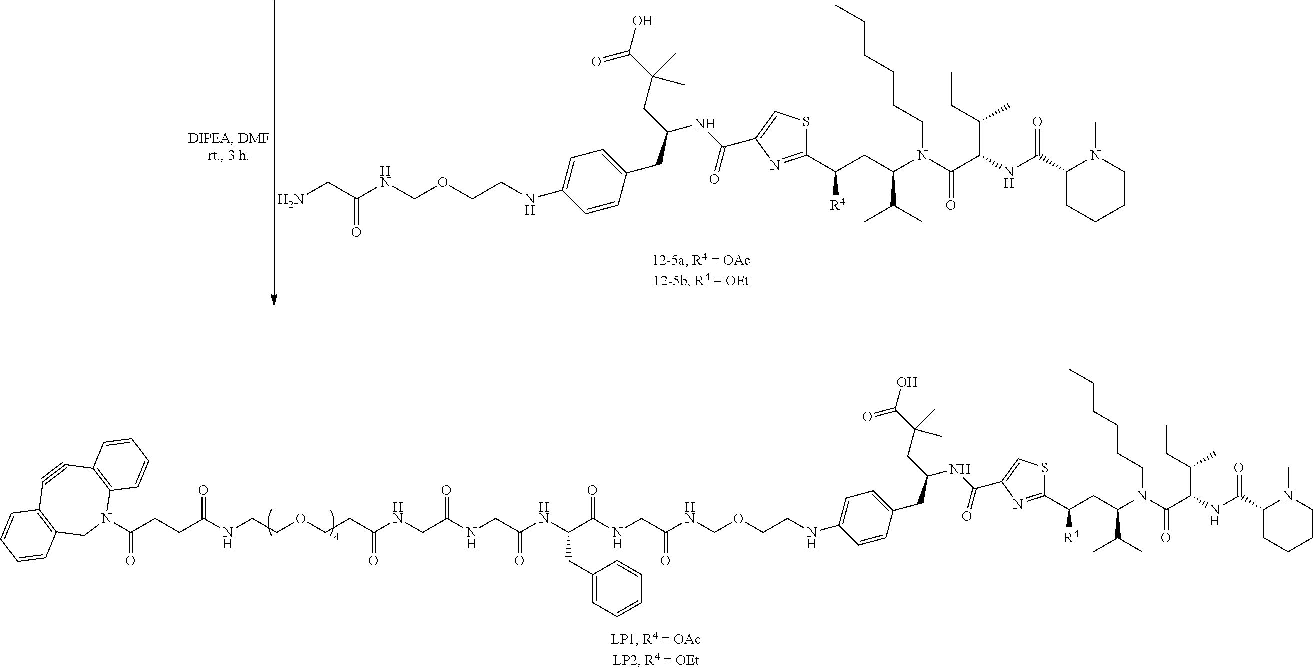

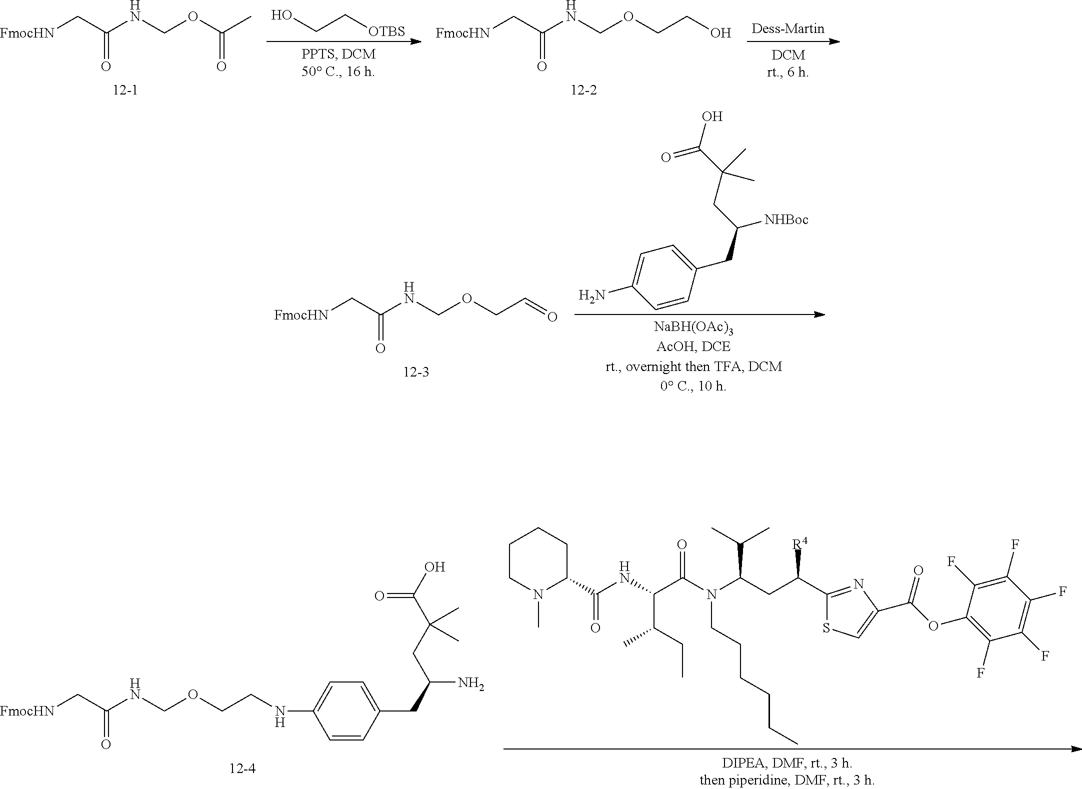

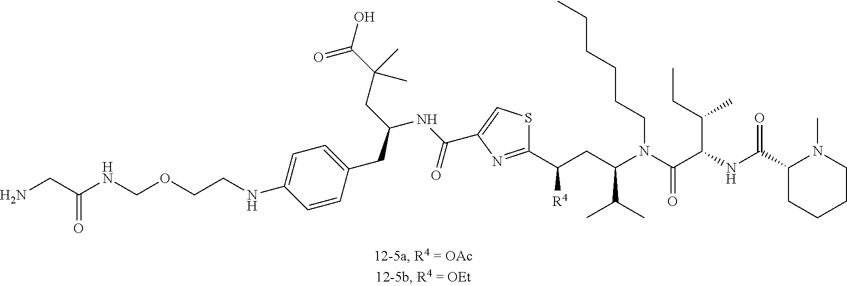

### (9H-fluoren-9-yl)methyl (2-(((2-hydroxyethoxy)methyl)amino)-2-oxoethyl)carbamate (12-2)

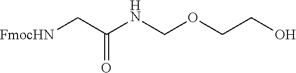

To a solution of commercial compound 12-1 (CAS: 1599440-06-8, 5.5 g, 15 mmol) in DCM (3 mL) was added 2-[[tert-butyl(dimethyl)silyl]oxy]ethan-1-ol (2.7 g, 30 mmol) and pyridinium p-toluenesulfonate (PPTS, 0.76 g, 3.0 mmol) and the reaction mixture was stirred at ° C. under nitrogen atmosphere for sixteen hours, which was monitored by LCMS. The volatiles were removed in vacuo and the residue was purified by reversed phase flash chromatography (0-100% acetonitrile in aq. TFA (0.01%)) to give compound 12-2 (1.5 g, 27% yield) as a white solid. ESI m/z 393.2 (M+H)⁺. ¹H NMR (400 MHz, DMSO-d₆) δ 8.55 (t, J=5.6 Hz, 1H), 7.89 (d, J=7.6 Hz, 2H), 7.85 (d, J=7.2 Hz, 2H), 7.44-7.39 (m, 2H), 7.38-7.32 (m, 2H), 6.82-6.76 (m, 1H), 6.28 (s, 2H), 4.58-4.53 (m, 2H), 3.54 (d, J=6.0 Hz, 2H), 3.48-3.45 (m, 2H), 3.41-3.38 (m, 3H), 3.12 (br s, 1H) ppm.

### (9H-fluoren-9-yl)methyl (2-oxo-2-(((2-oxoethoxy)methyl)amino)ethyl)carbamate (12-3)

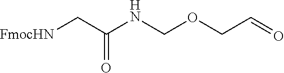

To a solution of compound 12-2 (0.30 g, 0.81 mmol) in DCM (10 mL) was added Dess-Martin Periodinane (DMP) (0.69 g, 1.6 mmol) and the reaction mixture was stirred at room temperature under nitrogen atmosphere for six hours, which was monitored by LCMS. The resulting mixture was diluted with sat. aq. sodium thiosulfate (30 mL) and the mixture was extracted with DCM (3×30 mL). The combined organic solution was washed with brine (30 mL), dried over anhydrous sodium sulfate, filtered, and concentrated in vacuo to give aldehyde 12-3 (0.20 g, 67% yield) as a white solid. ESI m/z 391.2 (M+H)⁺.

### Rac-(R)-5-(4-((1-(9H-fluoren-9-yl)-3,6-dioxo-2,9-dioxa-4,7-diazaundecan-11-yl)amino)phenyl)-4-amino-2,2-dimethylpentanoic acid (12-4)

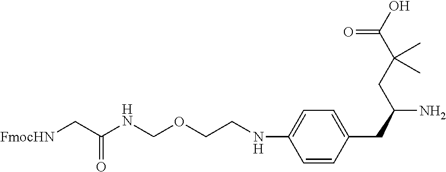

To a solution of compound 12-3 (0.91 g, 2.5 mmol) in DCE (15 mL) was added Boc-TupC (1.0 g, 3.0 mmol) and acetic acid (0.1 mL, 5 drops). The mixture was stirred at room temperature for ten minutes before the addition of sodium triacetoxyborohydride (1.1 g, 5.0 mmol). The reaction mixture was stirred at room temperature for sixteen hours, which was monitored by LCMS. The resulting mixture was concentrated in vacuo and the residue was purified by reversed phase flash chromatography (0-70% acetonitrile in aq. ammonium bicarbonate (10 mM)) to give compound Boc-12-4 (0.31 g, ESI m/z 711.2 (M+Na)⁺) as a light yellow solid, which was dissolved in DCM (2 mL). To the resulting solution was added a solution of TFA in DCM (VTFA:VDcm=1:5, 10 mL) dropwise at 0° C. over two minutes. The resulting mixture was stirred at 0° C. for ten hours until Boc was totally removed according to LCMS. The volatiles were removed in vacuo to give 12-4 (0.12 g, 8.2% yield from 12-3) as a white solid. ESI m/z 589.2 (M+H)⁺.

### Rac-(R)-4-(2-((1S,3S)-1-acetoxy-3-((2R,3R)—N-hexyl-3-methyl-2-((S)-1-methylpiperidine-2-carboxamido)pentanamido)-4-methylpentyl)thiazole-4-carboxamido)-acid (12-4)

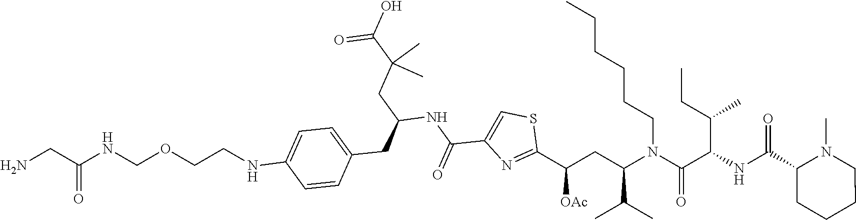 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCOCNC(=O)CN)cc2)CC(C)(C)C(=O)O)cs1)C(C)C]

Following Alternative General Procedure VI (then Fmoc removal) starting from compound 5-4a (0.16 g, 0.20 mmol) and treating with compound 12-4 (0.12 g, 0.20 mmol), compound 12-5a (75 mg, 40% yield) was obtained as a white solid. ESI m/z 957.3 (M+H)⁺. ¹H NMR (400 MHz, DMSO-d₆) δ 8.30 (t, J=6.8 Hz, 1H), 8.17 (s, 1H), 7.70 (d, J=9.2 Hz, 1H), 7.69-7.60 (m, 1H), 6.87 (d, J=8.4 Hz, 2H), 6.47 (d, J=8.4 Hz, 2H), 5.67-5.63 (m, 1H), 5.39-5.32 (m, 1H), 4.59 (d, J=6.4 Hz, 2H), 4.48 (t, J=8.8 Hz, 1H), 4.23-4.17 (m, 1H), 3.73-3.68 (m, 2H), 3.51 (d, J=6.0 Hz, 3H), 3.14-3.08 (m, 4H), 2.88-2.80 (m, 2H), 2.68-2.63 (m, 1H), 2.57-2.55 (m, 1H), 2.34-2.23 (m, 2H), 2.14 (s, 3H), 2.06 (s, 3H), 1.97-1.92 (m, 1H), 1.91-1.80 (m, 4H), 1.67-1.58 (m, 4H), 1.56-1.50 (m, 3H), 1.43-1.36 (m, 2H), 1.33-1.26 (m, 6H), 1.06-1.02 (m, 7H), 0.95 (d, J=6.4 Hz, 3H), 0.88-0.80 (m, 11H), 0.70-0.66 (m, 3H) ppm.

### Rac-(R)-5-(4-((24(2-aminoacetamido)methoxy)ethyl)amino)phenyl)-4-(2-((1S,3S)-1-ethoxy-3-((2R,3R)—N-hexyl-3-methyl-2-((S)-1-methylpiperidine-2-carboxamido)pentanamido)-4-methylpentyl)thiazole-4-carboxamido)-2,2-dimethylpentanoic acid (12-5b)

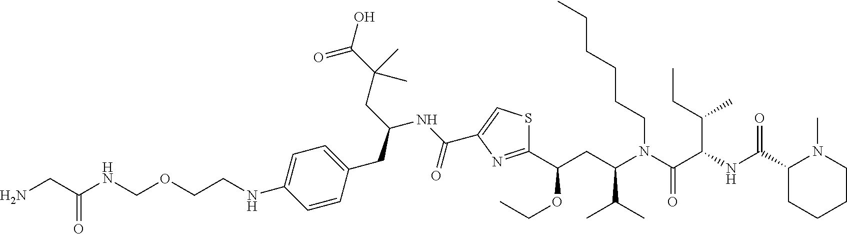 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCOCNC(=O)CN)cc2)CC(C)(C)C(=O)O)cs1)C(C)C]

Following Alternative General Procedure VI (then Fmoc removal) starting from 5-4b (76 mg, 0.10 mmol) treated with compound 12-4 (60 mg, 0.10 mmol), compound 12-5b (60 mg, 64% yield) was obtained as a white solid. ESI m/z 944.6 (M+H)⁺.

### 2,5-dioxopyrrolidin-1-yl (2S)-2-(2-{2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]acetamido}acetamido)-3-phenylpropanoate (12-7)

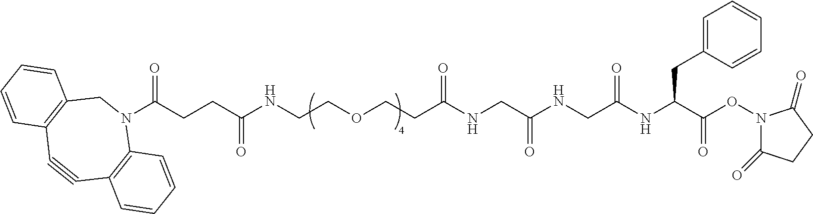

To a solution of compound 12-6 (41 mg, 50 μmol) in DCM (3 mL) was added HOSu (12 mg, 0.10 mmol) and EDCI (20 mg, 0.10 mmol) and the mixture was stirred at room temperature for three hours, which was monitored by LCMS. The resulting mixture was quenched with water (30 mL) and extracted with DCM (3×30 mL). The combined organic solution was washed with brine (30 mL), dried over anhydrous sodium sulfate, filtered, and concentrated in vacuo to give crude compound 12-7 (46 mg, crude) as a white solid, which was used in the next step without further purification. ESI m/z 911.4 (M+H)⁺.

### LP1

### (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-(4-{[2-({2-[(2S)-2-(2-{2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]acetamido}acetamido)-3-phenylpropanamido]acetamido}methoxy)ethyl]amino}phenyl)-2,2-dimethylpentanoic acid (LP1)

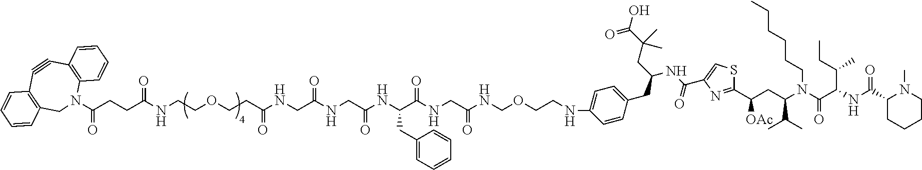

To a solution of compound 12-5a (40 mg, 42 μmol) in DMF (3 mL) was added DIPEA (17 mg, 0.13 mmol) and compound 12-7 (46 mg, 50 μmol, crude) obtained above, and the reaction mixture was stirred at room temperature for three hours, which was monitored by LCMS. The resulting mixture was directly purified by prep-HPLC (10-95% acetonitrile in aq. ammonium bicarbonate (10 mM)) to give linker-payload LP1 (10 mg, 14% yield) as a white solid. ESI m/z 876.2 (M/2+H)⁺. ¹H NMR (400 MHz, DMSO-d₆) δ 8.54 (t, J=6.8 Hz, 1H), 8.32 (t, J=5.2 Hz, 1H), 8.21-8.19 (m, 1H), 8.18-8.16 (m, 1H), 8.14 (d, J=8.0 Hz, 1H), 8.03 (t, J=5.6 Hz, 1H), 7.77 (t, J=5.6 Hz, 2H), 7.67-7.65 (m, 1H), 7.62-7.59 (m, 1H), 7.50-7.46 (m, 2H), 7.39-7.35 (m, 2H), 7.27-7.16 (m, 5H), 6.91-6.85 (m, 2H), 6.52-6.45 (m, 2H), 5.65 (d, J=11.6 Hz, 1H), 5.35 (br s, 1H), 5.06-5.00 (m, 1H), 4.60-4.55 (m, 2H), 4.52-4.46 (m, 2H), 4.25-4.19 (m, 1H), 3.79-3.75 (m, 2H), 3.74-3.67 (m, 9H), 3.66-3.65 (m, 1H), 3.63-3.57 (m, 8H), 3.48-3.45 (m, 13H), 3.31-3.27 (m, 2H), 3.14-3.03 (m, 4H), 2.85-2.79 (m, 1H), 2.65-2.61 (m, 1H), 2.59-2.55 (m, 2H), 2.39 (t, J=6.8 Hz, 2H), 2.27-2.20 (m, 2H), 2.14 (s, 3H), 2.08 (br s, 3H), 2.01-1.93 (m, 2H), 1.89-1.83 (m, 2H), 1.79-1.72 (m, 2H), 1.65-1.59 (m, 3H), 1.56-1.50 (m, 2H), 1.32-1.23 (m, 8H), 1.07-1.02 (m, 7H), (t, J=6.4 Hz, 3H), 0.89-0.79 (m, 12H), 0.70-0.65 (m, 3H) ppm.

### LP2

### (4S)-5-(4-{[2-({2-[(2S)-2-(2-{2-11-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]acetamido}acetamido)-3-phenylpropanamido]acetamido}methoxy)ethyl]amino}phenyl)-4-({2-[(1R,3R)-1-ethoxy-3-1(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic acid (LP2)

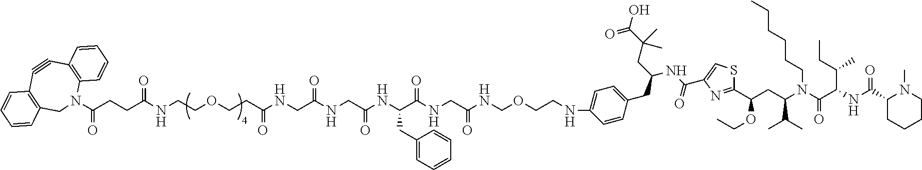

Following a similar procedure as LP4 except starting from 12-5b (40 mg, 42 μmol) instead of 12-5a, linker-payload LP2 (10 mg, 14% yield) was obtained as a white solid. ESI m/z 870.2 (M/2+H)⁺. ¹H NMR (400 MHz, DMSO-d₆) δ 8.56 (t, J=6.8 Hz, 1H), 8.34 (t, J=5.6 Hz, 1H), 8.20 (t, J=4.8 Hz, 1H), 8.18-8.14 (m, 2H), 8.06-8.01 (m, 1H), 7.82-7.78 (m, 1H), 7.68 (d, J=8.0 Hz, 1H), 7.62 (d, J=6.8 Hz, 1H), 7.51-7.45 (m, 3H), 7.40-7.29 (m, 4H), 7.26-7.17 (m, 4H), 6.90-6.84 (m, 2H), 6.51-6.45 (m, 2H), 5.38 (br s, 1H), 5.05-5.00 (m, 1H), 4.59-4.55 (m, 2H), 4.53-4.47 (m, 2H), 4.32-4.27 (m, 1H), 4.22-4.16 (br s, 1H), 3.79-3.67 (m, 8H), 3.63-3.56 (m, 7H), 3.52-3.49 (m, 4H), 3.47-3.44 (m, 13H), 3.31-3.27 (m, 3H), 3.14-3.03 (m, 5H), 2.83-2.76 (m, 1H), 2.63-2.55 (m, 4H), 2.38 (t, J=6.4 Hz, 2H), 2.27-2.19 (m, 1H), 2.08 (br s, 3H), 2.03-1.90 (m, 5H), 1.79-1.72 (m, 2H), 1.67-1.58 (m, 4H), 1.32-1.28 (m, 5H), 1.23 (br s, 1H), 1.20-1.12 (m, 4H), 1.05-1.00 (m, 7H), 0.92-0.79 (m, 16H), 0.70-0.65 (m, 3H) ppm.

### LP3

### (4S)-5-(4-{2-[(2S)-2-(2-{2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]acetamido}acetamido)-3-phenylpropanamido]acetamido}phenyl)-4-({2-[(1R,3R)-1-ethoxy-3-[(2S,3S)—N-hexyl-2-{[(2R)-1-(2-hydroxyethyl)piperidin-2-yl]formamido}-3-methylpentanamido]-4-methylpentyl]-1,3-thiazol-4-yl}formamido)-2,2-dimethylpentanoic acid (LP3)

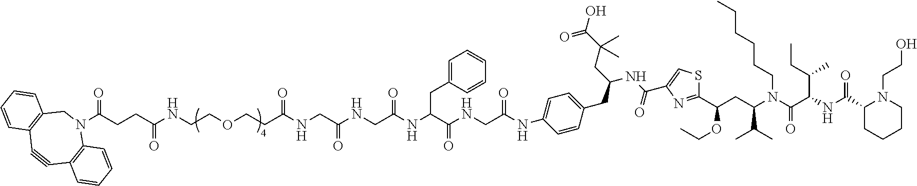

To a solution of payload PA-12 aka PA9 (15 mg, 17 μmol) in DMF (2 mL) was added crude compound 12-7 (18 mg, obtained above) and DIPEA (4.0 mg, 31 μmol) and the reaction mixture was stirred at room temperature for an hour, which was monitored by LCMS. The volatiles were removed in vacuo and the residue was purified by reversed phase flash chromatography (0-100% acetonitrile in aq. ammonium bicarbonate (10 mM)) to give linker-payload LP3 (2.5 mg, 10% yield) as a white solid. ESI m/z 848.6 (M/2+H)⁺. ¹H NMR (400 MHz, DMSO-d₆) δ 9.78 (s, 1H), 8.40 (s, 1H), 8.21-8.17 (m, 3H), 8.05 (s, 1H), 7.80-7.76 (m, 2H), 7.68 (d, J=7.8 Hz, 1H), 7.62 (d, J=7.4 Hz, 1H), 7.57-7.43 (m, 5H), 7.40-7.35 (m, 4H), 7.28-7.14 (m, 4H), 7.10 (d, J=8.4 Hz, 1H), 5.03 (d, J=14.1 Hz, 1H), 4.62-4.45 (m, 3H), 4.31-4.23 (m, 2H), 3.86-3.85 (m, 2H), 3.84-3.76 (m, 3H), 3.69 (d, J=5.9 Hz, 2H), 3.62-3.58 (m, 6H), 3.52-3.41 (m, 18H), 3.09-3.06 (m, 4H), 2.94-2.74 (m, 3H), 2.67 (s, 1H), 2.56-2.50 (m, 3H), 2.08-1.84 (m, 9H), 1.81-1.59 (m, 4H), 1.58-1.50 (m, 3H), 1.32 (s, 6H), 1.24 (br s, 2H), 1.18-1.14 (m, 4H), 1.04 (s, 3H), 1.02 (s, 3H), 0.88-0.86 (m, 14H), 0.69 (br s, 3H) ppm.

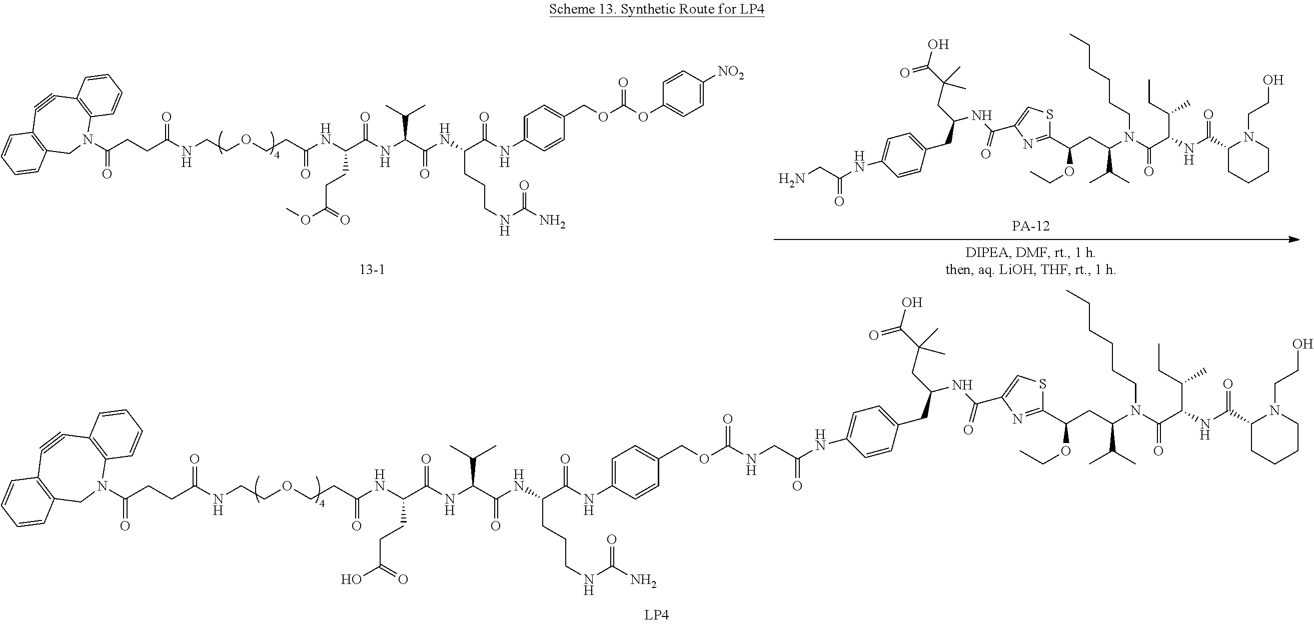

### LP4

### (4S)-5-[4-(2-{[({4-[(2S)-2-[(2S)-2-[(2S)-2-11-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-carboxybutanamido]-3-methylbutanamido]-5-(carbamoylamino)pentanamido]phenyl}methoxy)carbonyl]amino}acetamido)phenyl]-4-({2-[(1R,3R)-1-ethoxy-3-[(2S,3S)—N-hexyl-2-{[(2R)-1-(2-hydroxyethyl)piperidin-2-yl]formamido}-3-methylpentanamido]-4-methylpentyl]-1,3-thiazol-4-yl}formamido)-2,2-dimethylpentanoic acid (LP4)

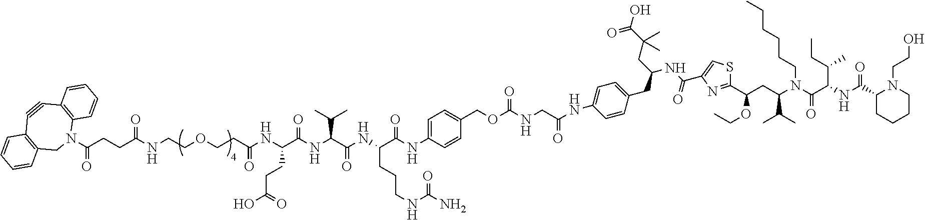

To a solution of payload PA-12 aka PA9 (10 mg, 11 μmol) in DMF (2 mL) was added compound 13-1 (14 mg, 11 μmol) and DIPEA (3.0 mg, 23 μmol) and the reaction mixture was stirred at room temperature for an hour, which was monitored by LCMS. The volatiles were removed in vacuo and the residue was separated by reversed phase flash chromatography (0-100% acetonitrile in water) to give a white solid (11 mg, ESI m/z 661.8 (M/3+H)⁺), which was dissolved in THF (2 mL). To the resulting solution was added aq. lithium hydroxide (0.01 M, 1 mL) and the mixture was stirred at room temperature for an hour. The resulting mixture was concentrated in vacuo and the residual mixture was purified by reversed phase flash chromatography (0-100% acetonitrile in water) to give linker-payload LP4 (1 mg, 5% yield from PA-12 aka PA9) as a white solid. ESI m/z 657.3 (M/3+H)⁺. ¹H NMR (500 MHz, DMSO-d₆) δ 10.04 (s, 1H), 9.88 (s, 1H), 8.19 (s, 1H), 8.13 (s, 1H), 8.09-8.08 (m, 1H), 7.76 (s, 3H), 7.68 (d, J=7.8 Hz, 1H), 7.64-7.54 (m, 3H), 7.53-7.41 (m, 6H), 7.33-7.30 (m, 4H), 7.09 (d, J=8.0 Hz, 2H), 6.01 (s, 1H), 5.44 (s, 2H), 5.08-4.93 (m, 3H), 4.53 (s, 2H), 4.32-4.20 (m, 5H), 3.75 (s, 2H), 3.65-3.56 (m, 2H), 3.47-3.40 (m, 10H), 3.33-3.30 (m, 15H), 3.09-2.91 (m, 3H), 2.64 (s, 1H), 2.38-2.36 (m, 4H), 2.22-2.20 (m, 6H), 2.00-1.90 (m, 7H), 1.80-1.60 (m, 5H), 1.58-1.40 (m, 7H), 1.30 (s, 6H), 1.24 (s, 3H), 1.21-1.13 (m, 4H), 1.06 (s, 3H), 1.04 (s, 3H), 0.87-0.82 (m, 20H), 0.69 (br s, 3H) ppm.

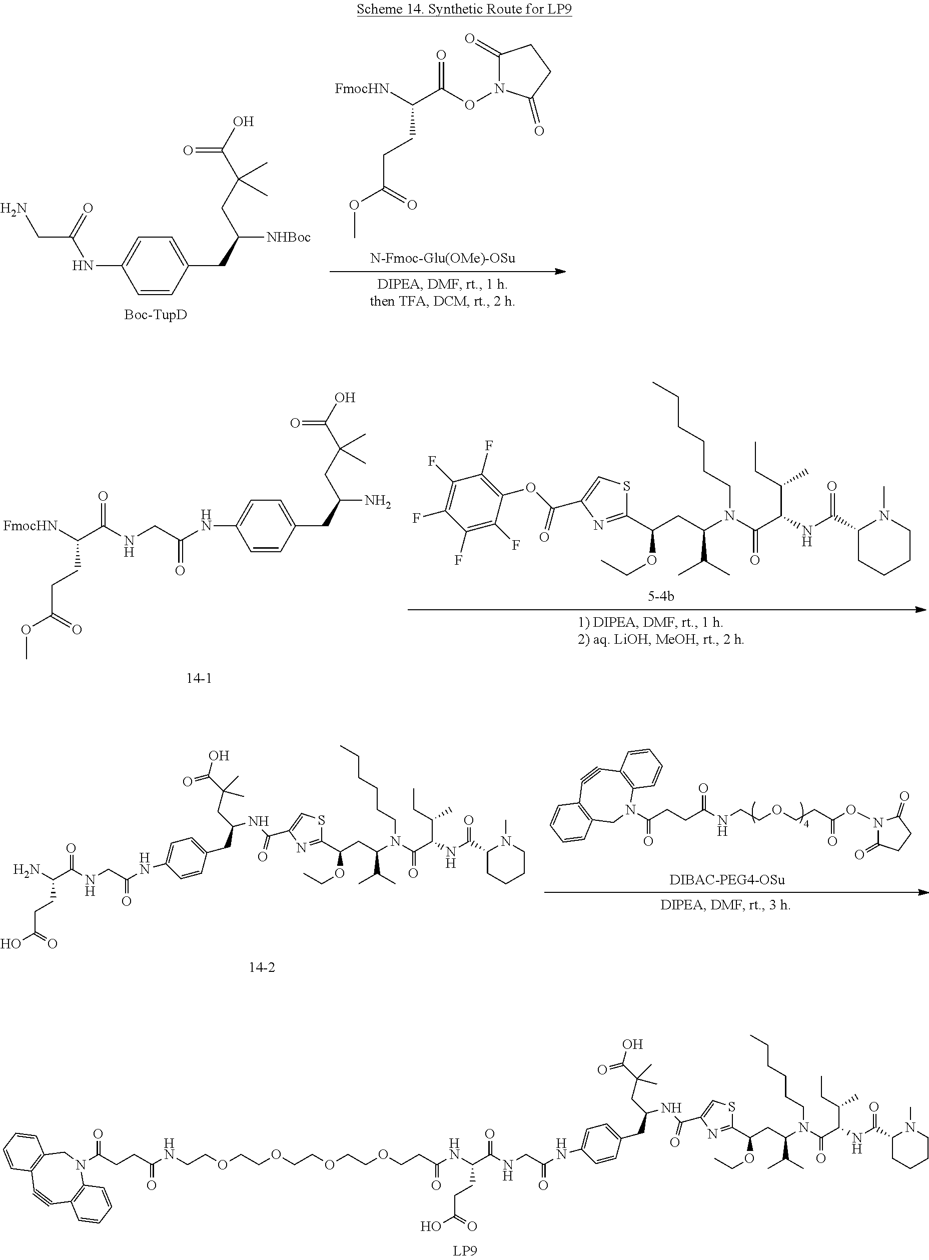

### LP9

### (S)-5-(4-(2-((RS)-2-((((9H-fluoren-9-yl)methoxy)carbonyl)amino)-5-methoxy-5-oxopentanamido)acetamido)phenyl)-4-amino-2,2-dimethylpentanoic acid (14-1)

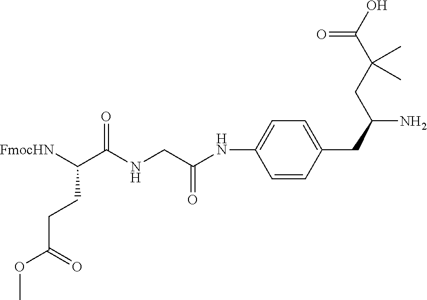

To a mixture of compound Boc-TupD (0.25 g, 0.64 mmol) in DMF (4 mL) was added N-Fmoc-Glu(OMe)-OSu (0.31 g, 0.64 mmol) and DIPEA (0.25 g, 1.9 mmol) and the reaction mixture was stirred at room temperature for an hour, which was monitored by LCMS. The resulting mixture was directly separated by reversed phase flash chromatography (0-100% acetonitrile in aq. ammonium bicarbonate (10 mM)) to give compound Boc-14-1 (0.10 g, 21% yield) as a white solid. ESI m/z 659.3 (M-Boc+H)⁺. To a solution of compound Boc-14-1 (0.16 g, 0.21 mmol) in DCM (5 mL) was added TFA (1 mL) and the reaction mixture was stirred at room temperature for three hours until Boc was totally removed according to LCMS. The volatiles were removed in vacuo and the residue was purified by reversed phase flash chromatography (0-30% acetonitrile in aq. TFA (0.1%)) to give 14-1 (0.10 g, 72% yield) as a white solid. ESI m/z 659.3 (M+H)⁺.

### (S)-5-(4-(2-((RS)-2-amino-4-carboxybutanamido)acetamido)phenyl)-4-(2-((1R,3R)-1-ethoxy-3-((2S,3S)—N-hexyl-3-methyl-2-((R)-1-methylpiperidine-2-carboxamido)pentanamido)-4-methylpentyl)thiazole-4-carboxamido)-2,2-dimethylpentanoic acid (14-2)

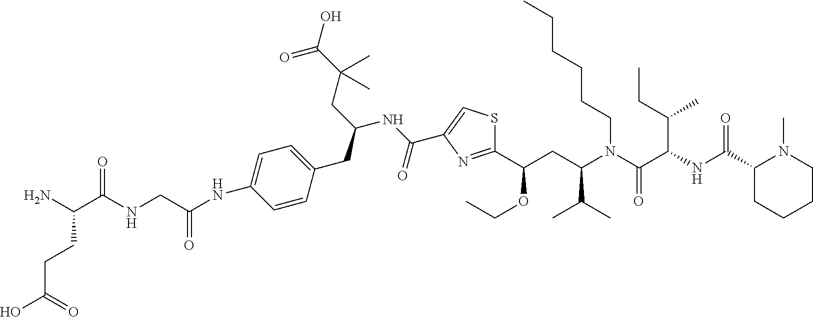 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CNC(=O)[C@@H](N)CCC(=O)O)cc2)CC(C)(C)C(=O)O)cs1)C(C)C]

Following Alternative General Procedure VI starting from 5-4b (0.12 g, 0.15 mmol) and treating with 14-1 (0.10 g, 0.15 mmol), the intermediate containing Fmoc and the methyl ester (0.12 g, ESI m/z 618.5 (M/2+H)⁺) was obtained as a white solid, which was then dissolved in methanol (5 mL). To the resulting solution was added aq. lithium hydroxide (0.1 M, 5 mL) and the reaction mixture was stirred at room temperature for two hours, which was monitored by LCMS. The resulting mixture was concentrated in vacuo and the residual mixture was purified by reversed phase flash chromatography (0-100% acetonitrile in aq. TFA (0.1%)) to give compound 14-2 (20 mg, 13% yield from 5-4b) as a white solid. ESI m/z 500.3 (M/2+H)⁺, 999.2 (M+H)⁺.

### (4S)-5-(4-{2-[(2S)-2-[1-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-carboxybutanamido]acetamido}phenyl)-4-({2-[(1R,3R)-1-ethoxy-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic acid (LP9)

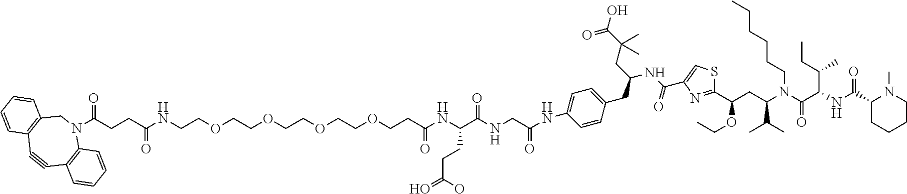 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CNC(=O)[C@H](CCC(=O)O)NC(=O)CCOCCOCCOCCOCCNC(=O)CCC(=O)N3Cc4ccccc4C#Cc4ccccc43)cc2)CC(C)(C)C(=O)O)cs1)C(C)C]

To a mixture of compound 14-2 (20 mg, 20 μmol) in DMF (5 mL) was added DIBAC-PEG4-OSu (13 mg, 20 μmol) and DIPEA (8.0 mg, 62 μmol) and the reaction mixture was stirred at room temperature for an hour, which was monitored by LCMS. The resulting mixture was directly separated by prep-HPLC (5-95% acetonitrile in aq. ammonium bicarbonate (10 mM)) to give linker-payload LP9 (2.0 mg, 7% yield) as a white solid. ESI m/z 767.3 (M/2+H)⁺. ¹H NMR (400 MHz, DMSO-d₆) δ 9.88 (s, 1H), 8.20-8.10 (m, 4H), 7.81-7.75 (m, 2H), 7.68 (dd, J=7.5, 1.5 Hz, 1H), 7.62 (dd, J=6.8, 1.2 Hz, 1H), 7.51-7.45 (m, 4H), 7.39-7.33 (m, 2H), 7.33-7.27 (m, 2H), 7.08 (d, J=8.5 Hz, 2H), 5.03 (d, J=13.8 Hz, 2H), 4.54-4.47 (m, 2H), 4.33-4.15 (m, 6H), 3.84-3.80 (m, 2H), 3.62-3.56 (m, 3H), 3.52-3.43 (m, 12H), 3.09-3.06 (m, 2H), 2.69-2.65 (m, 2H), 2.35-2.31 (m, 3H), 2.24-2.18 (m, 4H), 2.14-2.08 (m, 4H), 2.03-1.90 (m, 8H), 1.80-1.69 (m, 6H), 1.68-1.58 (m, 5H), 1.30 (s, 6H), 1.20-1.13 (m, 5H), 1.05 (s, 3H), 1.03 (s, 3H), 0.92-0.65 (m, 17H) ppm.

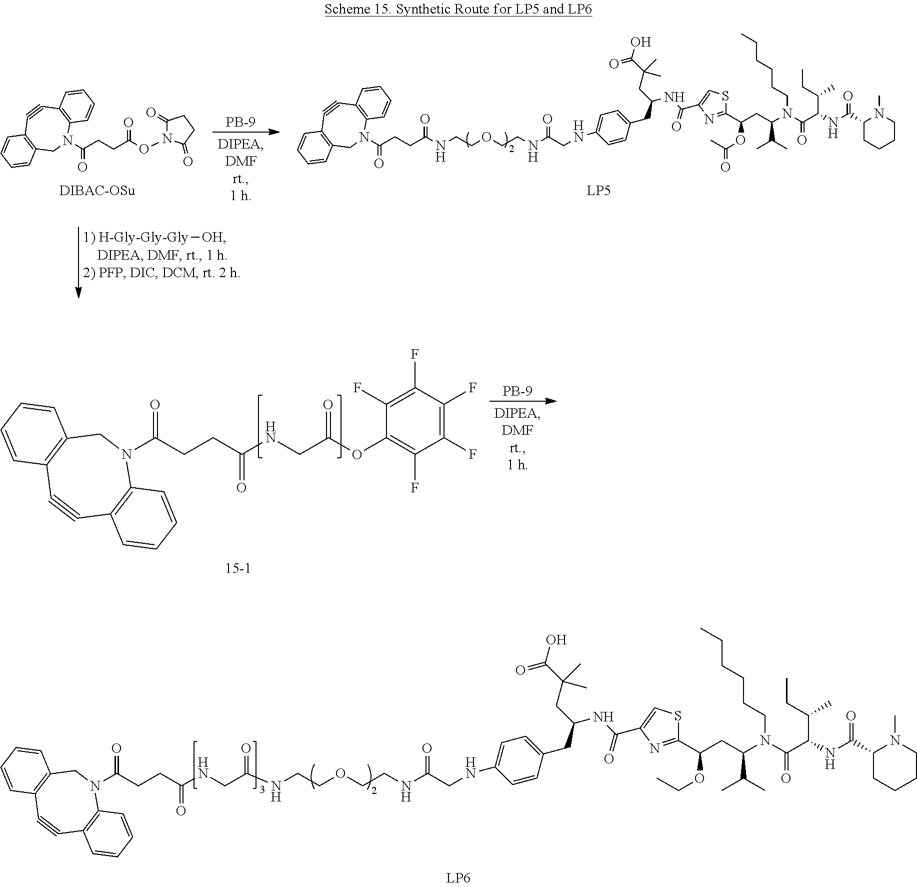

### LP5

### (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-14-({[(2-{2-[2-(4-{2-azatricyclo[10.4.0.0^{4,9}]hexadeca-1(12),4(9),5,7,13,15-hexaen-10-yn-2-yl}-4-oxobutanamido)ethoxy]ethoxy}ethyl)carbamoyl]methyl}amino)phenyl]-2,2-dimethylpentanoic acid (LP5)

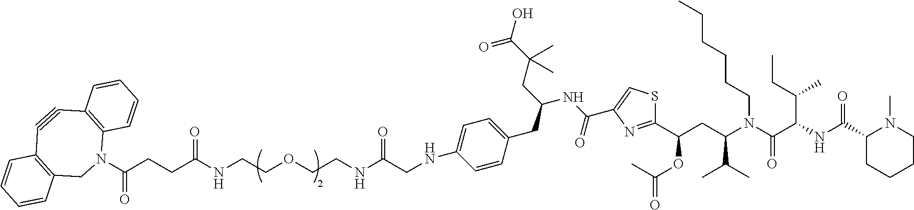

To a mixture of payload PB-9 aka PA20 (15 mg, 15 μmol) in DMF (2 mL) was added DIBAC-OSu (6.0 mg, 15 μmol) and DIPEA (6.0 mg, 45 μmol) and the reaction mixture was stirred at room temperature for an hour, which was monitored by LCMS. The resulting mixture was directly purified by reversed phase flash chromatography (0-100% acetonitrile in aq. ammonium bicarbonate (10 mM)) to give linker-payload LP5 (4.0 mg, 21% yield) as a white solid. ESI m/z 651.8 (M/2+H)⁺. ¹H NMR (400 MHz, DMSO-d₆) δ 8.16 (s, 1H), 7.83 (t, J=5.6 Hz, 1H), 7.76 (t, J=5.6 Hz, 1H), 7.69-7.66 (m, 1H), 7.64-7.58 (m, 2H), 7.52-7.44 (m, 3H), 7.42-7.27 (m, 4H), 6.89 (d, J=8.3 Hz, 2H), 6.42 (d, J=8.3 Hz, 2H), 5.77 (t, J=5.7 Hz, 1H), 5.65 (d, J=13.0 Hz, 1H), (d, J=14.0 Hz, 2H), 4.55-4.40 (m, 1H), 4.26-4.15 (m, 2H), 3.62-3.33 (m, 9H), 3.30-3.25 (m, 3H), 3.23-3.18 (m, 2H), 3.11-3.04 (m, 2H), 2.70-2.60 (m, 2H), 2.30-2.18 (m, 3H), 2.17-1.95 (m, 8H), 1.92-1.71 (m, 6H), 1.70-1.41 (m, 8H), 1.38-1.09 (m, 10H), 1.05 (s, 3H), 1.03 (s, 3H), 1.00-0.65 (m, 15H) ppm.

### LP6

### 2,3,4,5,6-pentafluorophenyl 2-{2-[2-(4-{2-azatricyclo[10.4.0.0^{4,9}]hexadeca-1(12),4(9),5,7,13,15-hexaen-10-yn-2-yl}-4-oxobutanamido)acetamido]acetamido}acetate (15-1)

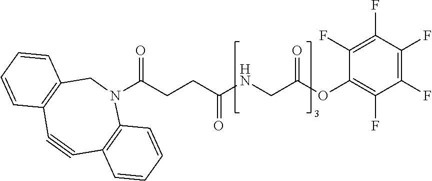

To a mixture of H-Gly-Gly-Gly-OH (43 mg, 0.22 mmol) in DMF (8 mL) was added DIBAC-OSu (90 mg, 0.22 mmol) and DIPEA (87 mg, 0.67 mmol) and the mixture was stirred at room temperature for an hour, which was monitored by LCMS. The resulting mixture was directly separated by reversed phase flash chromatography (0-100% acetonitrile in aq. ammonium bicarbonate (10 mM)) to give a white solid (60 mg, ESI m/z 477.3 (M+H)⁺), half of which was dissolved in DCM (3 mL). To the resulting solution was added pentafluorophenol (24 mg, 0.13 mmol) and DIC (16 mg, 0.13 mmol) and the reaction mixture was stirred at room temperature for two hours, which was monitored by LCMS. The resulting mixture was concentrated in vacuo to crude 15-1 (29 mg, crude), which was used in the next step without further purification. ESI m/z 643.2 (M+H)⁺.

### (4S)-4-({2-[(1R,3R)-1-(acetyloxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-5-[4-({[(2-{2-[2-(2-{2-[2-(4-{2-azatricyclo[10.4.0.0^{4,9}]hexadeca-1(12),4(9),5,7,13,15-hexaen-10-yn-2-yl}-4-oxobutanamido)acetamido]acetamido}acetamido)ethoxy]ethoxy}ethyl)carbamoyl]methyl}a mino)phenyl]-2,2-dimethylpentanoic acid (LP6)

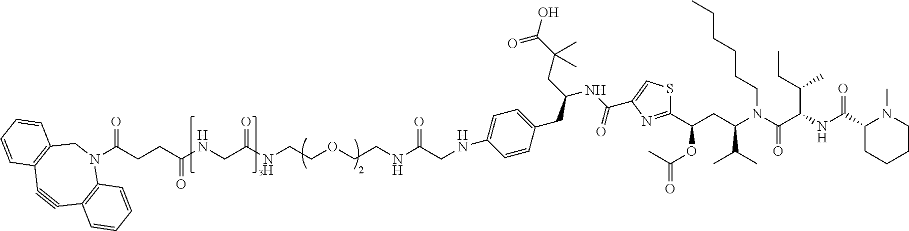

To a mixture of payload PB-9 aka PA20 (15 mg, 15 μmol) in DMF (5 mL) was added crude compound 15-1 (10 mg) obtained above and DIPEA (5.8 mg, 45 μmol) successively, and the reaction mixture was stirred at room temperature for an hour, which was monitored by LCMS. The resulting mixture was directly separated by prep-HPLC (5-95% acetonitrile in aq. ammonium bicarbonate (10 mM)) to give linker-payload LP6 (9.6 mg, 44% yield) as a white solid. ESI m/z 737.3 (M/2+H)⁺. ¹H NMR (400 MHz, DMSO-d₆) δ 8.17-8.00 (m, 4H), 7.89-7.80 (m, 2H), 7.68 (d, J=7.0 Hz, 2H), 7.61 (d, J=7.0 Hz, 2H), 7.54-7.44 (m, 3H), 7.40-7.27 (m, 3H), 6.89 (d, J=7.3 Hz, 2H), 6.43 (d, J=7.1 Hz, 2H), 5.77 (t, J=5.7 Hz, 1H), 5.65 (d, J=11.1 Hz, 1H), 5.02 (d, J=14.5 Hz, 2H), 4.55-4.40 (m, 1H), 4.28-4.11 (m, 2H), 3.73-3.30 (m, 16H), 3.25-3.15 (m, 5H), 2.70-2.59 (m, 3H), 2.34-2.02 (m, 11H), 1.93-1.40 (m, 14H), 1.38-1.14 (m, 10H), 1.05 (s, 3H), 1.04 (s, 3H), 1.00-0.65 (m, 15H) ppm.

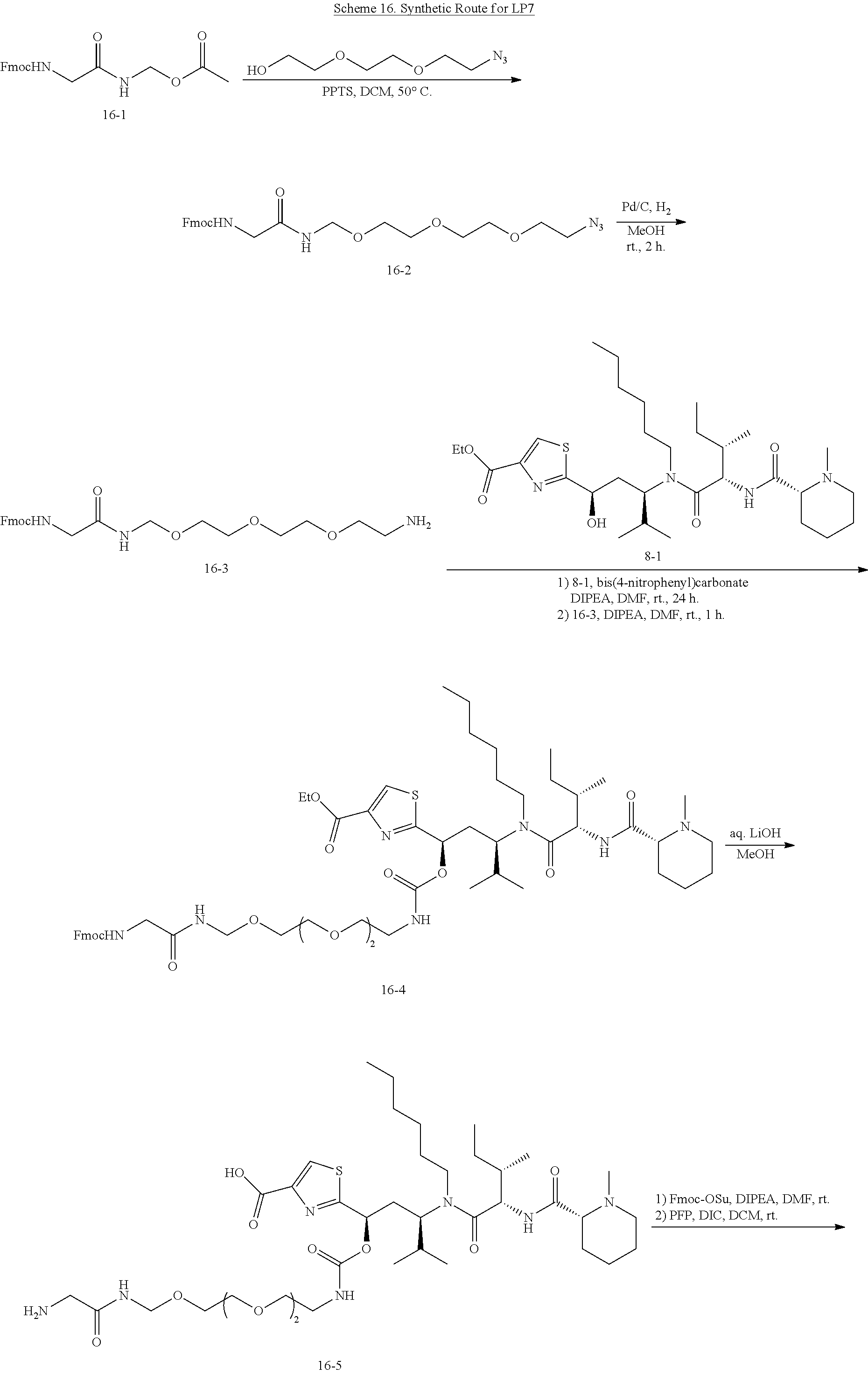

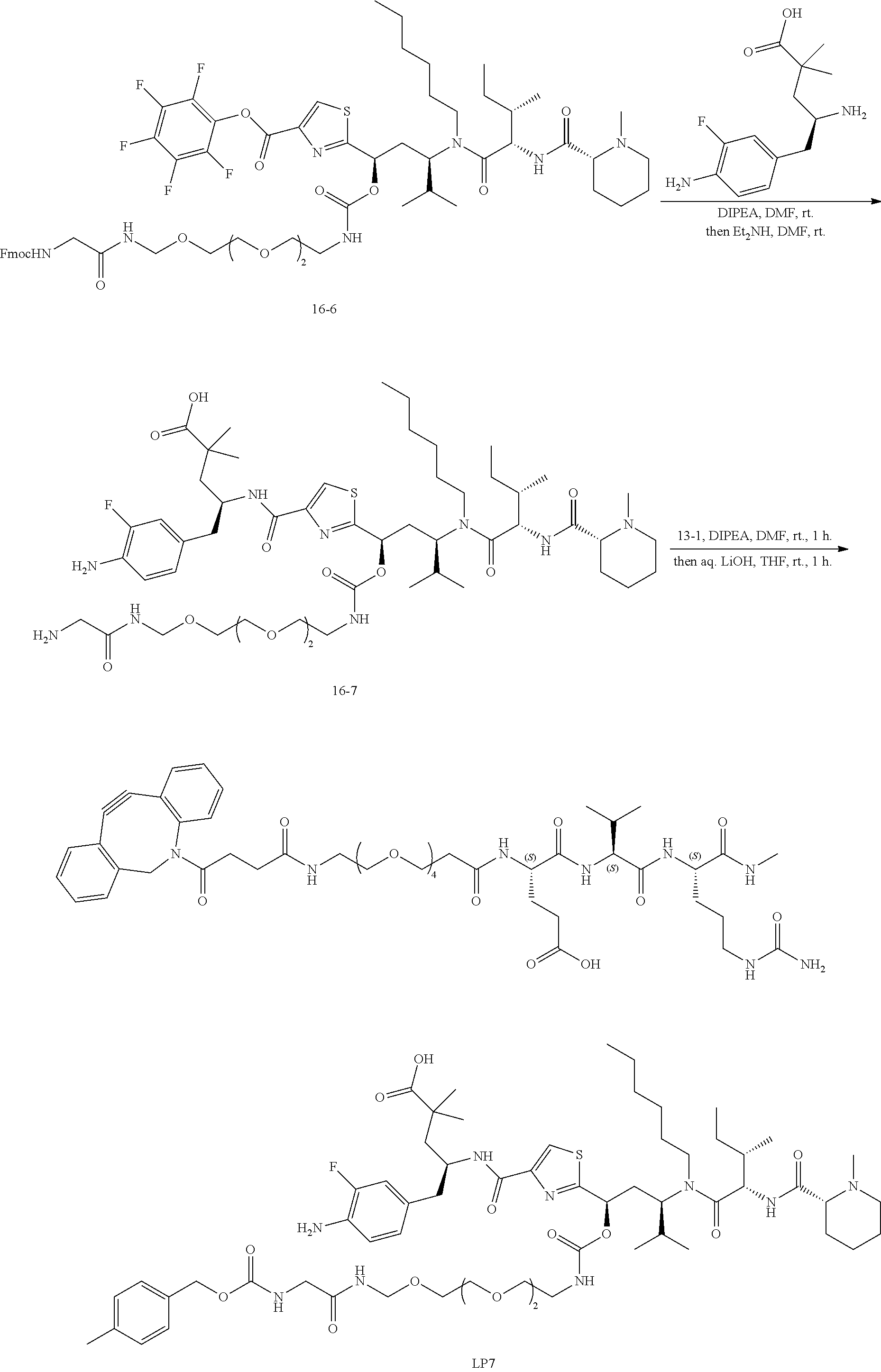

### LP7

### (9H-fluoren-9-yl)methyl (13-azido-2-oxo-5,8,11-trioxa-3-azatridecyl)carbamate (16-2)

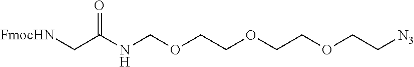

A 100 mL sealed tube was charged with azido-PEG3-OH (CAS: 86520-52-7, 0.20 g, 1.3 mmol), compound 16-1 (0.42 g, 1.1 mmol), PPTS (29 mg, 0.11 mmol), and DCM (20 mL) and the tube was then sealed. The reaction mixture was stirred at 50° C. in the sealed tube overnight. After cooling to room temperature, the resulting solution was poured into MTBE (100 mL) and a white solid precipitated. The suspension was filtered. The white filter cake was collected and dried in air to give compound 16-2 (0.45 g, 81% yield) as a white solid. ESI m/z 484.1 (M+H)⁺.

### (9H-fluoren-9-yl)methyl (13-amino-2-oxo-5,8,11-trioxa-3-azatridecyl)carbamate (16-3)

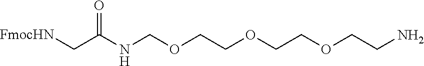

To a solution of compound 16-2 (0.48 g, 0.99 mmol) in methanol (10 mL) was added palladium-carbon (10% Pd, 50 mg) under nitrogen and the reaction mixture was stirred under a hydrogen atmosphere at room temperature for two hours, which was monitored by LCMS. The resulting suspension was filtered through Celite and the filtrate was concentrated in vacuo. The crude product was purified by silica gel flash chromatography (50% ethyl acetate in petroleum ether) to give 16-3 (0.13 g, 30% yield) as a yellow oil. ESI m/z 458.3 (M+H)⁺.

### Ethyl 2-017R,19R)-1-((((9H-fluoren-9-yl)methoxy)carbonyl)amino)-19-isopropyl-20-(((R)-1-methylpiperidine-2-carbonyl)-L-isoleucyl)-2,15-dioxo-5,8,11,16-tetraoxa-3,14,20-triazahexacosan-17-yl)thiazole-4-carboxylate (16-4)

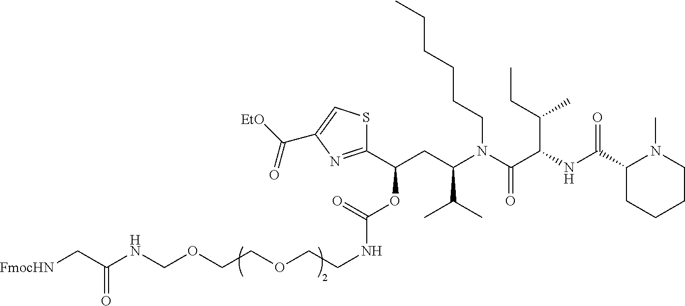

Following Alternative General Procedure VIII starting from compound 8-1 (0.28 g, mmol) and treating with amine 16-3, compound 16-4 (78 mg, 16% yield) was obtained as a white solid. ESI m/z 1078.6 (M+H)⁺.

### 2-((17R,19R)-1-amino-19-isopropyl-20-(((R)-1-methylpiperidine-2-carbonyl)-L-isoleucyl)-2,15-dioxo-5,8,11,16-tetraoxa-3,14,20-triazahexacosan-17-yl)thiazole-4-carboxylic acid (16-5)

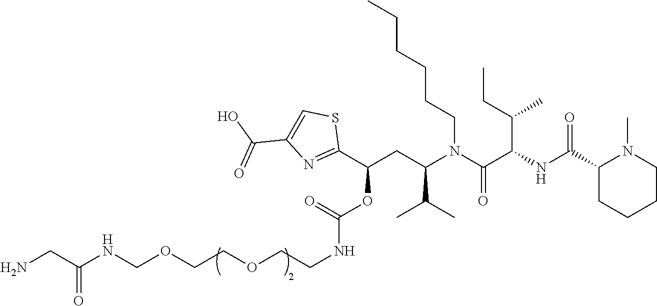

Following Alternative General Procedure III starting from compound 16-4 (78 mg, 72 μmol), compound 16-5 (Fmoc also removed, 38 mg, 63% yield) was obtained as a white solid. ESI m/z 414.9 (M/2+H)⁺.

### Perfluorophenyl 2-(((17R,19R)-1-((((9H-fluoren-9-yl)methoxy)carbonyl)amino)-19-isopropyl-20-(((R)-1-methylpiperidine-2-carbonyl)-L-isoleucyl)-2,15-dioxo-5,8,11,16-tetraoxa-3,14,20-triazahexacosan-17-yl)thiazole-4-carboxylate (16-6)

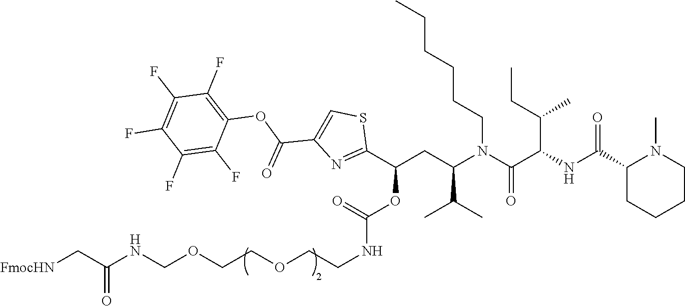

To a solution of compound 16-5 (38 mg, 46 μmol) in DMF (5 mL) was added Fmoc-OSu (CAS: 82911-69-1, 16 mg, 46 μmol) and DIPEA (18 mg, 0.14 mmol) and the reaction mixture was stirred at room temperature for an hour until compound 16-5 was totally protected by Fmoc according to LCMS. The resulting mixture was directly separated by reversed phase flash chromatography (0-100% acetonitrile in aq. ammonium bicarbonate (10 mM)) to give a white solid (40 mg, ESI m/z 1050.6 (M+H)⁺), which was used in the next step without further purification. Following Alternative General Procedure V using the Fmoc-protected 16-5 obtained above, compound 16-6 (24 mg, 43% yield from 16-5) was obtained as a white solid. ESI m/z 1216.5 (M+H)⁺.

### (S)-4-(2-((17R,19R)-1-amino-19-isopropyl-20-(((R)-1-methylpiperidine-2-carbonyl)-L-isoleucyl)-2,15-dioxo-5,8,11,16-tetraoxa-3,14,20-triazahexacosan-17-yl)thiazole-4-carboxamido)-5-(4-amino-3-fluorophenyl)-2,2-dimethylpentanoic acid (16-7)

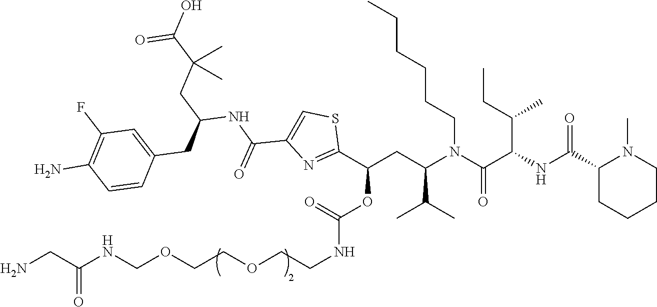

Following Alternative General Procedure VI (then Fmoc removal) starting from 16-6 (24 mg, 20 μmol) and treating with TupA, compound 16-7 (12 mg, 55% yield from 16-6) was obtained as a white solid. ESI m/z 1065.4 (M+H)⁺, 532.8 (M/2+H)⁺.

### (4S)-5-(4-amino-3-fluorophenyl)-4-({2-[(1R,3R)-1-({12-(2-{2-[(2-{[({4-[(2S)-2-1(2S)-2-[(2S)-2-11-(4-{2-azatricyclo[10.4.0.0^{4,9}]hexadeca-1(12),4(9),5,7,13,15-hexaen-10-yn-2-yl}-4-oxobutanamido)-3,6,9,12-tetraoxapentadecan-15-amido]-4-carboxybutanamido]-3-methylbutanamido]-5-(carbamoylamino)pentanamido]phenyl}methoxy)carbonyl]amino}acetamido)methoxy]etho xy}ethoxy)ethyl]carbamoyl}oxy)-3-[(2S,3S)—N-hexyl-3-methyl-2-{[(2R)-1-methylpiperidin-2-yl]formamido}pentanamido]-4-methylpentyl]-1,3-thiazol-4-yl}formamido)-2,2-dimethylpentanoic acid (LP7)

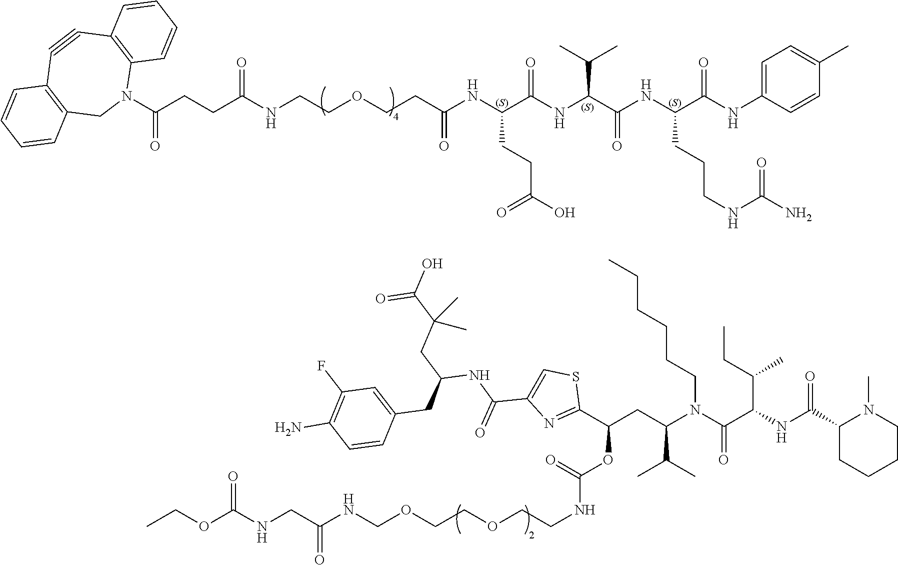

Following a similar procedure as LP7 except starting from 16-7 (13 mg, 11 μmol) instead of PA-12 aka PA9, linker-payload LP7 (11 mg, 47% yield) was obtained as a white solid. ESI m/z 711.8 (M/3+H)⁺. ¹H NMR (400 MHz, DMSO-d₆) δ 10.04 (s, 1H), 8.69-8.62 (m, 2H), 8.19 (d, J=7.4 Hz, 1H), 8.13 (s, 1H), 8.09 (d, J=8.4 Hz, 1H), 7.80-7.72 (m, 3H), 7.70-7.65 (m, 2H), 7.64-7.57 (m, 4H), 7.53-7.42 (m, 5H), 7.39-7.34 (m, 2H), 7.33-7.26 (m, 3H), 6.79-6.71 (m, 2H), 6.67-6.62 (m, 2H), 6.03-5.96 (m, 2H), 5.61-5.54 (m, 2H), 5.44 (s, 2H), 5.03 (d, J=13.9 Hz, 2H), 4.99-4.90 (m, 4H), 4.54 (d, J=6.6 Hz, 2H), 4.51-4.44 (m, 2H), 4.41-4.30 (m, 4H), 4.27-4.15 (m, 4H), 3.64-3.50 (m, 14H), 3.16-2.91 (m, 9H), 2.64-2.56 (m, 3H), 2.44-2.30 (m, 5H), 2.27-2.18 (m, 4H), 2.17-2.04 (m, 5H), 2.03-1.93 (m, 4H), 1.92-1.74 (m, 7H), 1.74-1.54 (m, 9H), 1.48-1.33 (m, 6H), 1.31-1.21 (m, 6H), 1.07 (s, 3H), 1.06 (s, 3H), 0.94 (d, J=6.2 Hz, 3H), 0.89-0.64 (m, 18H) ppm.

### ADC Conjugation

### General Procedure for Conjugation

This example demonstrates a method for conjugation of a maleimide-spacer-payload to inter-chain cysteines of an antibody or antigen-binding fragment via the formation of a thioether bond.

Conjugation through antibody cysteines were performed in two steps using methods similar to those for making Adcetris®-like ADCs (see, Mol. Pharm. 2015, 12(6), 1863-71). A monoclonal antibody (mAb) (10 mg/mL in 50 mM HEPES, 150 mM NaCl) at pH 7-8 was reduced with 1 mM dithiothreitol (6 molar equiv to antibody) or TCEP (2.5 molar equivalents to antibody) at 37° C. for 30 min. After gel filtration (G-25, pH 6.3, sodium acetate), a linker-payload at 1-10 mg/mL in DMSO was added to the reduced antibody, and the reaction was allowed to stir for 3-14 h at rt. The resulting mixture was purified by SEC to generate pure ADC.

### General Procedure for Site-Specific Conjugation

This example demonstrates a method for site-specific conjugation of a cyclooctyne-linker-payload to an antibody or antigen-binding fragment thereof.

In this example, the site-specific conjugates were produced in two steps. The first step was microbial transglutaminase (MTG) based enzymatic attachment of a small molecule, such as an azido-PEG3-amine, to the antibody having a N297Q mutation (hereinafter “MTG-based” conjugation). The second step used the attachment of a cyclooctyne-spacer-payload to the azido-functionalized antibody via a [2+3] cycloaddition, for example, the 1,3-dipolar cycloaddition between an azide and a cyclooctyne (aka copper-free click chemistry). See, Baskin, J. M.; Prescher, J. A.; Laughlin, S. T.; Agard, N. J.; Chang, P. V.; Miller, I. A.; Lo, A.; Codelli, J. A.; Bertozzi, C. R. PNAS 2007, 104 (43), 16793-7. This process provided site-specific and stoichiometric conjugates in about 50-80% isolated yield.

### Step 1: Preparation of an Azido-Functionalized Antibody

Aglycosylated human antibody IgG (IgG1, IgG4, etc.) or a human IgG1 isotype with a N297Q mutation, in PBS (pH 6.5-8.0) was mixed with ≥200 molar equivalents of azido-PEG3-amine (ZP3A, MW=218.26 g/mol). The resulting solution was mixed with MTG (EC 2.3.2.13 from Zedira, Darmstadt, Germany, or ACTIVA TI which contains Maltodextrin from Ajinomoto, Japan) (25 U/mL; 5U MTG per mg of antibody) resulting in a final concentration of 0.5-5 mg/mL antibody, and the solution was then incubated at 37° C. for 4-24 h while gently shaking. The reaction was monitored by ESI-MS. Upon reaction completion, the excess amine and MTG were removed by SEC or protein A column chromatography, to generate the azido-functionalized antibody. The product was characterized by SDS-PAGE.

In certain experiments, the N297Q antibody (24 mg) in 7 mL potassium-free PBS buffer (pH 7.3) was incubated with >200 molar equivalents of the azido-PEG3-amine ZP3A (MW=218.26) in the presence of MTG (0.350 mL, 35 U, mTGase, Zedira, Darmstadt, Germany). The reaction was incubated at 37° C. overnight while gently mixing. Excess azido-PEG₃-amine and mTGase were removed by size exclusion chromatography (SEC, Superdex 200 PG, GE Healthcare).

Step 2: Preparation of site-specific conjugates by a [2+3] click reaction between the azido-functionalized transglutaminase-modified antibodies (IgG1, IgG4, etc.) and cyclooctyne containing linker-payloads (LPs). In general, an azido-functionalized aglycosylated antibody-LP conjugate was prepared by incubating the azido-functionalized transglutaminase-modified antibody (1 mg) in 1 mL of an aqueous medium (e.g., PBS, PBS containing 5% glycerol, HBS) with ≥6 molar equivalents of an LP dissolved in a suitable organic solvent (e.g., DMSO, DMF or DMA; reaction mixture contains 10-20% organic solvent, v/v) at 24° C. to 32° C. for over three hours. The progress of the reaction was monitored by ESI-MS. Absence of azido-functionalized or transglutaminase-modified antibody (mAb-PEG₃-N₃) indicated completion of the conjugation. The excess linker-payload (LP) and organic solvent were removed by SEC (Waters, Superdex 200 Increase, 1.0×30 cm, GE Healthcare, flow rate 0.8 mg/mL, PBS, pH 7.2) eluting with PBS, or via protein A column chromatography via elution with acidic buffer followed by neutralization with Tris (pH 8.0). The purified conjugate was analyzed by SEC, SDS-PAGE, and ESI-MS.

In certain examples, the azido-functionalized antibody (1 mg) in 0.800 mL PBSg (PBS, 5% glycerol, pH 7.4) was treated with six equivalents of DIBAC-Suc-PEG₄-VC-PABC-payload (10 mg/mL in DMSO) for six hours at rt and the excess linker payload (LP) was removed by size exclusion chromatography (SEC, Superdex 200 HR, GE Healthcare). The final product was concentrated by ultra-centrifugation and characterized by UV, SEC, SDS-PAGE, and/or ESI-MS.

Alternative General Procedure for ADC Conjugations

A site-specific antibody conjugate with linker-payload (LP) was prepared by incubating an anti-HER2 antibody or antigen binding fragment thereof (1-3 mg/mL) in an aqueous medium (e.g., BupH) with ten molar equivalents of an LP dissolved in a suitable organic solvent, for example, DMSO. The reaction mixture contained 5-15% organic solvent, v/v and was incubated at 25° C. for 16 h (DIBAC) and 72 h (COT). The linker-payload was mixed with buffer first and then added into the antibody solution with slow stirring. The progress of the reaction was monitored by SDS-PAGE and PR-HPLC. If the conjugation was incomplete on Day 1, then another LP equivalent was added to the mixture and the absence of the antibody or antigen binding fragment thereof indicated the completion of the conjugation. The excess amount of the LP and organic solvent were removed via desalting the column with BupH (pH 7.4). The final product was characterized by SDS-PAGE, RP-HPLC, and LC-MS.

| | | Rₜ for | | |
|---|---|---|---|---|
| | | Light | | ΔRₜ |
| ADC | DAR | Chain | Rₜ for Heavy Chain | (HCmax-LC) |
| ADC1 | 3.84 | 9.49 | 11.640, 12.677 | 3.19 |
| ADC2 | 3.94 | 9.48 | 11.490; 12.733 | 3.25 |
| ADC3 | 3.84 | 9.49 | 11.817; 12.747; 13.370 | 3.88 |
| ADC4 | 3.96 | 9.49 | 11.660; 13.060 | 3.57 |

Alternative RP-HPLC for ADC Analysis

The intact mass for an ADC sample by RP-HPLC was performed to determine whether the LPs had been fully conjugated and was also used to calculate the average DAR. Each sample was treated with Dithiothreitol (DTT, 0.5M) and then incubated at 37° C. for 30 min prior to the RP-HPLC analysis. The RP-HPLC was performed using a Thermo UltiMate™ 3000 instrument, on a)(Bridge Protein BEH C4 column (300 Å, 2.5 μm, 4.6×100 mm; Cat No. 186009137), and the column oven was heated to 65° C. Each testing sample (10-20 pg, 10 μL) was loaded and run at the flow rate of 1 mL/min using different gradients of Mobile Phase A (100% ddH₂O with 0.1% TFA) and Mobile Phase B (80% ACN, 20% IPA with 0.1% TFA) and monitored at λ 280 nm using a Thermo DAD-3000 RS Rapid Separation Diode Array Detector.

### Preparation of ADCs 1-37

Step 1: In this step, the antibody was site-specifically functionalized at glutamine residues with an azido-alkyl amine. Specifically, anti-Her2 human IgG antibody containing an N297Q mutation (TRSQ) or isotype control antibody containing the same mutation (CTRL) was mixed with excess, for example, 20-100 molar equivalents of the appropriate azido-alkyl amine. The resulting solution was mixed with transglutaminase (1U mTG per mg of antibody, Millipore-Sigma) resulting in a final concentration of the antibody at 1-20 mg/mL. The reaction mixture was incubated at 25-37° C. for four to twenty-four hours while gently shaking. Reaction progress was monitored by ESI-MS. Upon completion, excess amine and mTG were removed by size exclusion chromatography (SEC) or protein A column chromatography. The conjugate was characterized by UV-Vis, SEC, and ESI-MS.

Step 2: In this step, the antibody produced in Step 1 was conjugated with a linker payload via a cycloaddition reaction. Specifically, the azido-functionalized antibody from Step 1 was incubated (1-20 mg/mL) in PBS (pH7.4) with 10-20 molar equivalents of a linker-payload dissolved in an organic solvent (e.g., DMSO or DMA (10 mg/mL)) to obtain a reaction mixture that is approximately 5-15% organic solvent (v/v) at 25-37° C. for one to forty-eight hours while gently shaking. The reaction was monitored by ESI-MS. Upon completion, the excess amount of linker-payload and protein aggregates were removed by size exclusion chromatography (SEC). The purified conjugate was concentrated, sterile filtered, and characterized by UV-Vis, SEC, and ESI-MS. Monomeric mAb purity was >99% by SEC.

### General Procedure for Characterization of Antibodies and ADCs

The purified conjugates were analyzed by SEC, ESI-MS, and SDS-PAGE.

### Characterization of ADC by SEC

Analytical SEC experiments used a Waters 1515 instrument, on a Superdex™ 200 Increase (1.0×30 cm) column at flow rate of 0.80 mL/min using PBS pH 7.2, and monitored at λ=280 nm using a Waters 2998 PDA. An analytic sample was composed of 200 μL PBS (pH 7.4) with 30-100 μL of test sample. Preparative SEC purifications can be performed using an AKTA Avant instrument from GE Healthcare on Superdex 200 PG (2.6×60 cm) column at a flow rate 2 mL/min eluting with PBS pH 7.2, and monitored at X, =280 nm. The SEC results typically indicated retention times for monomeric mAb and conjugates thereof with minimal aggregation or degradation.

### Characterization of ADC by LC-ESI-MS

Measurement of intact mass for the ADC samples by LC-ESI-MS was performed to determine drug-payload distribution profiles and to calculate the average drug:antibody ratio (DAR). Each testing sample (20-50 ng, 5 μL) was loaded onto an Acquity UPLC Protein BEH C₄ column (10K psi, 300 Å, 1.7 μm, 75 μm×100 mm; Cat No. 186003810). After desalting for 3 min, the protein was eluted and mass spectra was acquired by a Waters Synapt G2-Si mass spectrometer. Most site-specific ADCs have near 4DAR.

### Characterization of ADC by SDS-PAGE

SDS-PAGE was used to analyze the integrity and purity of the ADCs. In one method, SDS-PAGE conditions included non-reduced and reduced samples (2-4 μg) along with BenchMark Pre-Stained Protein Ladder (Invitrogen, cat #10748-010; L #1671922.) loaded per lane in (1.0 mm×10 well) Novex 4-20% Tris-Glycine Gel and were ran at 180 V, 300 mA, for 80 min. An analytical sample was prepared using Novex Tris-Glycine SDS buffer (2×) (Invitrogen, cat #LC2676) and the reduced sample was prepared with SDS sample buffer (2×) containing 10% 2-mercaptoethanol.

### In Vitro Plasma Stability

To determine the plasma stability of representative ADCs containing the tubulysin payloads or prodrug payloads, ADCs were incubated in vitro with plasma from different species, and the DAR was evaluated after incubation at physiological temperature (37° C.) for three days.

For the assay, each ADC sample in PBS buffer was added to fresh pooled male mouse, cynomologus monkey, rat, or human plasma, separately, at a final concentration of 50 μg/mL in a 96-well plate, and subsequently incubated at 37° C. for seventy-two hours. After incubation, each sample (100 μL final volume) was individually frozen at −80° C. until analysis.

Affinity capture of the ADCs from the plasma samples was carried out on a KingFisher 96 magnetic particle processor (Thermo Electron). First, biotinylated extracellular domain of human PRLR expressed with a myc-myc hexahistidine tag (hPRLR ecto-MMH 100 μg/mL) was immobilized on streptavidin paramagnetic beads (In vitrogen, Cat #60210). Each plasma sample containing tubulysin ADCs (100 μL) was mixed at 600 rpm with 100 μL of the beads (the commercial beads come in volume) at room temperature for two hours in a 96-well plate. The beads were then washed three times with 600 μL of HBS-EP (GE healthcare, Cat #BR100188), once with 600 μL of H₂O, and then once with 600 μL of 10% acetonitrile in water. Following the washes, tubulysin ADCs were eluted by incubating the beads with 70 μL of 1% formic acid in 30% acetonitrile/70% water for fifteen minutes at room temperature. Each eluate sample was then transferred into a v-bottom 96-well plate and was then reduced with 5 mM TCEP (Thermo Fisher, Cat #77720) at room temperature for twenty minutes.

The reduced tubulysin ADC samples (10 μL/sample) were injected onto a 1.7 μm BEH300 C4 column (Waters Corporation, Cat #186005589) coupled to a Waters Synapt G2-Si Mass Spectrometer. The flow rate was 0.1 mL/min (mobile phase A: 0.1% formic acid in water; mobile phase B: 0.1% formic acid in acetonitrile). The LC gradient started with 20% B and was increased to 35% B in sixteen minutes, and then reached 95% B in one minute.

The acquired spectra was deconvoluted using MaxEnt1 software (Waters Corporation) with the following parameters: Mass range: 20-30 kDa for the light chain, and 40-60 kDa for the heavy chain; m/z range: 700 Da-3000 Da; Resolution: 1.0 Da/channel; Width at half height: 1.0 Da; Minimum intensity ratios: 33%; Iteration max: 25.

Significant loss of linker-payloads was typically not observed from the tested ADCs after 72-hour incubation with human, mouse, rat, and cynomolgus monkey plasma. However, the acetyl groups of the tubulysin payloads or prodrug payloads were hydrolyzed to a hydroxyl group (−43 Da) with significant loss of toxicity. Therefore, the hydrolyzed species observed in the LCMS was considered as loss of drug. Drug:antibody ratio (DAR) can be calculated based on the relative abundance of different species of heavy chains.

$$\begin{matrix}Drug / antibody Ratio ( DAR ) = \\ 2 \times \end{matrix} \frac{\begin{matrix}2 \times Intensity ( heavy chain with 2 drugs ) + 1 \times Intensity \\ ( heavy chain with 1 drug )\end{matrix}}{\vert Sum Intensity ( Heavy chain with 2 , 1 and 0 drugs )}$$

Alternative Procedures for the Plasma Stability Test

Stock solutions were prepared at 10 mM in DMSO for the test compound. Aliquots of the stock solutions were diluted to 0.02 mM in 0.05 M sodium phosphate buffer containing 0.5% BSA as the dosing solution. Then 10 μL of the dosing solutions were dosed into 90 μL of pre-warmed plasma (37° C.) in singlet (n=1) in 96-well assay plates to reach a final test concentration of 2 μM. The plates were kept in a 37° C. water bath for the duration of the experiment. At each time point (0, 0.25, 1, 8, 24 h), 400 μL of acetonitrile (containing internal standard) was added into corresponding wells of the assay plates. After the final time point was quenched, the assay plates were shaken on a vibrator (IKA, MTS 2/4) for 10 min (600 rpm/min) and then centrifuged at 5594 g for 15 min (Thermo Multifuge×3R). Aliquots of the supernatant were taken and analyzed by LC-MS/MS. The peak area response ratio to internal standard (PARR) of the compounds at 0.25, 1, 8, and 24 h was compared to the PARR at time zero to determine the percent of test compound remaining at each time point. Half-lives (ti/2) were calculated using Excel software, fitting to a single-phase exponential decay equation.

### Testing of Tubulysin Payloads in Cell-Based Killing Assays

To test the ability of the disclosed tubulysin payloads or prodrug payloads to kill human cell lines, an in vitro cytotoxicity assay was performed. In vitro cytotoxicity of the disclosed payloads, as well as reference compounds, were evaluated using the CellTiter-Glo Assay Kit (Promega, Cat #G7573), in which the quantity of ATP present was used to determine the number of viable cells in culture. For the assay, HCT cells were seeded at 1000 cells/well on Nunclon white 96-well plates in complete growth medium (RPMI, 10% FBS, 100 units/mL Penicillin, 100 pg/mL streptomycin, 53 μg/mL glutatmine) and grown overnight at 37° C. in 5% CO₂. For cell viability curves, 1:3 serially diluted payloads were added to the cells at final concentrations ranging from 100 nM to 15 pM, including a no treatment control group, and were then incubated for five days. After the 5-day incubation, cells were incubated at room temperature with 100 μL of CellTiter-Glo reagents for ten minutes. Relative luminescence units (RLU) were determined on a Victor plate reader (PerkinElmer). The IC₅₀ values were determined from a four-parameter logistic equation over a 10-point response curve (GraphPad Prism). All IC₅₀ values were expressed in molar (M) concentration. The percent cell killing (% kill) at the maximum concentration tested was estimated from the following formula (100−% viable cells). Averages±standard deviation (SD) are included where replicate experiments were performed.

### Testing of Tubulysin Payloads in a Panel of SK-BR-3 Cell Lines

Anti-proliferation assays were performed using a SK-BR-3 human breast adenocarcinoma (pleural effusion) cell line. The cells were grown in McCoy's 5a Medium supplemented with 10% FBS, penicillin/streptomycin and L-glutamine. Cells were seeded 1000/well in 96-well plate in 80 μL complete growth media one day prior to adding ADCs and incubated at 37° C. in 5% CO₂ overnight.

The ADCs were 1:3 serially diluted ten points in assay media (Opti-MEM+0.1% BSA). The concentrations of the testing ADCs cover the range of 1 nM to ˜1000 nM and also started from different concentrations based on the cell killing potency in order to see EC₅₀ covers, leaving the last well (10^{th}) as blank (no ADC or compound). ADCs were first 1:3 serially diluted ten points in DMSO starting from 5.0 μM (the starting concentration of each ADC was different according to the EC₅₀s), leaving the last well as blank (containing only DMSO). 10 μL DMSO-diluted compound was transferred to 990 μL assay media (Opti-MEM+0.1% BSA) in a 96-well deep well dilution plate. 20 μL assay media-diluted ADC was added to cells. Cells were incubated at 37° C. in 5% CO₂ for six days (144 hrs). Plates were developed by adding 100 μL CTG reagent/well to the cells, CellTiter-Glo® from Promega Cat. No G7573), shaken at room temperature for 10 min, sealed with white adhesive bottom seal, and luminescence was read with Envision. Cell kill %=[1−(T144ₛₐₘₚₗₑ−T144_{blank})/(T144_{DMSO}−T144_{blank})]×100%, wherein T144 is the data at 144 h.

Alternative Cell-based Assay Procedures

HCT15

The cell line used in the anti-proliferation assays was HCT15, a human colon, colorectal carcinoma cell line. The HCT15 cells were grown in RPMI medium 1640+10% FBS. To run the assay, the cells (40 μL, 400 cells) were added to each well in a 384-well plate and incubated for twenty-four hours at 37° C. with 5% CO₂. One hour before adding compounds, freshly dilute Verapamil to 0.2 mg/ml in ddH₂O. Add 1 μL/well diluted Verapamil to Verapamil treatment wells and 1 μL/well ddH₂O to the non-Verapamil treatment wells. Return plates to the incubator at 37° C. with 5% CO₂. Then the cells were treated with test compounds at various concentrations using HPD300, and then normalize the DMSO to 0.5%. The control wells contained cells and the medium but lack the test compounds. The plates were incubated for 120 hours at 37° C. with 5% CO₂. CTG reagent was then added to the wells (25 μL). After the plates were shaken for 10 min and then incubated for 10 min at room temperature, paste the clear bottom with white back seal and record luminescence with Envision. The inhibition % was calculated according to the following equation: inhibition %=[1−(assay-blank)/(control-blank)]×100.

SK-BR-3

The cell line used in the anti-proliferation assays was SK-BR-3, a human breast, adenocarcinoma (pleural effusion) cell line. The cells were grown in McCoy's 5a Medium+10% FBS. To run the assay for payloads, the cells (40 μL, 700 cells) were added to each well in a 384-well plate and incubated for 24 hours at 37° C. with 5% CO₂. Then the cells were treated with test compounds at various concentrations using HPD300, and then normalize the DMSO to 0.5%. The control wells contain cells and the medium but lack the test compounds. The plates were incubated for 120 h at 37° C. with 5% CO₂. To run the assay for ADCs and some key payloads, the cells (80 μL, 1000 cells) were added to each well in a 96-well plate and incubated for twenty-four hours at 37° C. with 5% CO₂. Next, the ADCs or payloads were diluted with Opti-Mem with 0.1% BSA. The cells were treated with test compounds or ADCs (20 μL) at various concentrations in appropriate cell culture medium (total volume, 0.1 mL). The final concentration of DMSO was adjusted to 0.2% and the Opti-Mem with 0.1% BSA was adjusted to 5%. The control wells contained cells and the medium but lacked the test compounds. The plates were incubated for 144 h at 37° C. with 5% CO₂. CTG reagent was then added to the wells (25 μL/384-well or 100 μL/96-well). After the plates were shaken for 10 min and then incubated for 10 min at room temperature, paste the clear bottom with white back seal and record luminescence with Envision. The inhibition % was calculated according to the following equation: inhibition %=[1-(assay-blank)/(control-blank)]×100.

Table 5 provides the drug:antibody ratios (DARs) for ADC Nos. 1-4, along with the EC₅₀ results from the SKBR assays for the same conjugates.

TABLE 5
ADC Conjugation and SKBR Cell Kill Assay

| | | | ADC | | | |
|---|---|---|---|---|---|---|
| | | | | | | SKBR3 |
| Payload | Linker-payload | | | | | EC₅₀ |
| No. | No. | Name | Name | No. | DAR | (nM) |
| PA13 | LP3 | DIBAC-SUC-PEG4- | TRSQ-ZP3A- | 1 | 3.84 | 0.859 |
| | | GGFG-PA13 | LP3 | | | |
| PA13 | LP4 | DIBAC-SUC-PEG4- | TRSQ-ZP3A- | 2 | 3.96 | 2.515 |
| | | EVC-PAB-G-PA13 | LP4 | | | |
| PA14 | LP1 | DIBAC-SUC-PEG4- | TRSQ-ZP3A- | 3 | 3.84 | 0.155 |
| | | GGFG-NHCH2-PA14 | LP1 | | | |
| PA15 | LP2 | DIBAC-SUC-PEG4- | TRSQ-ZP3A- | 4 | 3.94 | 0.614 |
| | | GGFG-NHCH2-PA15 | LP2 | | | |

TABLE 6
In Vitro Activity and ADME of Tubulysin
Payloads Modified on Mep.

| | | | | Human | |
|---|---|---|---|---|---|
| | | HCT-15 | | plasma | |
| | | with | | stability | |
| Payload | HCT-15 | verapamil | SKBR3 | T1/2 | |
| No. | (nM) | (nM) | (nM) | (h) | PK |
| PA1 | >100 | >100 | | | |
| PA2 | >100 | | | | |
| PA3 | 7.698 | 0.741 | 1.460 | | Y |
| PA4 | 85.020 | 6.189 | >1 | | |
| PA5 | >100 | 15.886 | 18.324 | | |
| PA6 | 3.271 | 0.620 | 0.514 | | Y |
| PA7 | 0.122 | 0.037 | 0.038 | 65.94 | |
| PA8 | 0.072 | 0.029 | 0.017 | | |
| PA9 | 9.888 | 1.085 | 5.380 | | |
| PA10 | 31.720 | 2.947 | 5.327 | | |
| PA11 | 149.623 | 5.299 | 15.141 | | |
| PA12 | 72.068 | 4.730 | >100 | | |
| PA13 | 1.307 | 0.284 | 0.288 | 312.77 | Y |
| PA28 | 154.104 | | >100 | | |
| P15 | 1.658 | 0.190 | 0.068 | 15.07 | Y |
| P22 | 0.265 | 0.078 | 0.087 | | |

TABLE 7
In Vitro Activity and ADME of Tubulysin Payloads
(Modified on Tup).

| | | | | Human | |
|---|---|---|---|---|---|
| | | HCT-15 | | plasma | |
| | | with | | stability | |
| Payload | HCT-15 | verapamil | SKBR3 | T1/2 | |
| No. | (nM) | (nM) | (nM) | (h) | PK |
| PA14 | 0.618 | 0.147 | 0.158 | 71.03 | Y |
| PA15 | 1.699 | 0.393 | 0.314 | 103.06 | Y |
| PA16 | 0.062 | 0.033 | 0.019 | 326.73 | Y |
| PA17 | 1.390 | 0.090 | 0.131 | | |
| PA18 | 0.300 | 0.131 | 0.067 | | |
| PA19 | 10.764 | 1.800 | 1.845 | | |
| PA20 | | | | | |
| PA29 | 97.104 | 9.703 | 1.473 | | |
| PA30 | | | | | |
| P28 | 0.172 | 0.107 | 0.039 | | Y |

TABLE 8
In Vitro Activity and ADME of Tubulysin Payloads

| | | | | Human | |
|---|---|---|---|---|---|
| | | HCT-15 | | plasma | |
| | | with | | stability | |
| Payload | HCT-15 | verapamil | SKBR3 | T1/2 | |
| No. | (nM) | (nM) | (nM) | (h) | PK |
| PA21 | 13.752 | 1.727 | 0.703 | | |
| PA22 | 0.532 | Not reach | 0.044 | 63.51 | Y |
| | | bottom | | | |
| PA23 | >1000 | >1000 | 9.350 | | |
| PA24 | 0.944 | 0.255 | 0.135 | 43.41 | Y |
| PA25 | 0.477 | 0.158 | 1.598 | | |
| PA26 | 14.625 | 2.226 | 2.816 | | |
| PA27 | >100 | 33.559 | 63.896 | | |

TABLE 9
Properties of Linker-payloads

| | Linker | | | | | HPLC | |
|---|---|---|---|---|---|---|---|
| | Payload | | | | Purity | Rt | |
| LP# | Name | cLogP | MF | MW | (%) | (min) | ESI m/z |
| LP1 | DIBAC-SUC- | 3.46 | C₉₂H₁₂₉N₁₃O₁₉S | 1753.18 | >95 | 8.57 | 876.5 |
| | PEG4-GGFG- | | | | | | (M/2 + H) |
| | NHCH2-PA14 | | | | | | |
| LP2 | DIBAC-SUC- | 4.02 | C₉₂H₁₃₁N₁₃O₁₈S | 1739.19 | 99 | 8.95 | 870.2 |
| | PEG4-GGFG- | | | | | | (M/2 + H) |
| | NHCH2-PA15 | | | | | | |
| LP3 | DIBAC-SUC- | 4.03 | C₉₀H₁₂₆N₁₂O₁₈S | 1696.12 | 97 | 8.65 | 848.6 |
| | PEG4-GGFG- | | | | | | (M/2 + H) |
| | PA13 | | | | | | |
| LP4 | DIBAC-SUC- | 4.20 | C₁₀₁H₁₄₅N₁₅O₂₃S | 1969.41 | >95 | 7.48 | 985.2 |
| | PEG4-EVC- | | | | | | (M/2 + H) |
| | PAB-G-PA13 | | | | | | |
| LP5 | DIBAC-SUC- | 5.46 | C₇₁H₉₉N₉O₁₂S | 1302.68 | 98 | 7.54 | 651.8 |
| | PEG2-PA16 | | | | | | (M/2 + H) |
| LP6 | DIBAC-SUC- | 2.14 | C₇₇H₁₀₈N₁₂O₁₅S | 1473.84 | 96 | 8.11 | 737.3 |
| | GGG-PEG2- | | | | | | (M/2 + H) |
| | PA16 | | | | | | |
| LP7 | DIBAC-SUC- | 3.29 | C₁₀₆H₁₅₄FN₁₇O₂₆S | 2133.55 | >99 | 7.05 | 711.8 |
| | PEG4- | | | | | | (M/3 + H) |
| | EvcPAB-G- | | | | | | |
| | NHCH₂-PA25 | | | | | | |
| LP8 | COT-GGGG- | 4.54 | C₆₁H₉₁N₉O₁₄S | 1206.51 | 98 | 7.73 | 604.0 |
| | P22 | | | | | | (M/2 + H) |
| LP9 | DIBAC- | 4.53 | C₈₁H₁₁₆N₁₀O₁₇S | 1533.93 | >99 | 7.45 | 767.3 |
| | PEG4-E-P31 | | | | | | (M/2 + H) |
| LP10 | DIBAC- | 8.45 | C₉₂H₁₂₈FN₁₃O₁₉S | 1771.17 | 96 | 8.64 | 886.0 |
| | PEG4-vcPAB- | | | | | | (M/2 + H) |
| | P15 | | | | | | |
| LP11 | DIBAC- | 8.95 | C₉₂H₁₂₈N₁₂O₂₀S | 1754.16 | 95 | 8.46 | 877.5 |
| | PEG4-vcPAB- | | | | | | (M/2 + H) |
| | P22 | | | | | | |

What is claimed is:

1. A compound having the following formula 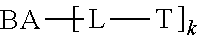
wherein
BA is a binding agent;
L is a linker covalently bound to BA and to T;
T is 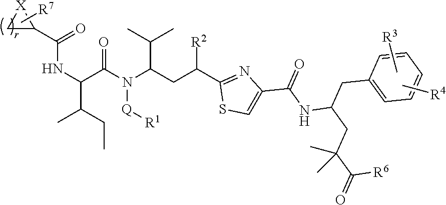
covalently bound to L, wherein
X is —O— or —NR⁵;
R⁵ is hydrogen, —CH₃, —(CH₂)₂—OH, —(CH₂)₂—NH₂, —CH₂—C(O)—OH,
—(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue; or
R⁵ is a covalent bond to L; or
R⁵ is —(CH₂)₂—OH or —CH₂—C(O)—OH with a covalent bond to L from the terminal oxygen in —(CH₂)₂—OH or —CH₂—C(O)—OH; or
R⁵ is —(CH₂)₂—NH₂, —(CH₂)₂—O—(CH₂)₂—NH₂, —(CH₂CH₂—O)₂—(CH₂)₂—NH₂,
—C(O)—CH₂—NH₂, a first N-terminal amino acid residue, or a first amino acid residue with a covalent bond to L from the nitrogen in any one of —(CH₂)₂—NH₂,
—(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, —O—C(O)—NH—(CH₂CH₂O)₃—CH₂NH—C(O)CH₂NH₂, or —O—C(O)—NH—(CH₂CH₂O)₂—(CH₂)₂OH; or
R² is —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH with a covalent bond to L from a terminal oxygen in any one of —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH; or
R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or
R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
R⁴ is hydrogen or —F;
R⁷ when present is —CH₃;
R⁶ is —OH, —NHCH₂C(O)OH, or —NH—C(O)OH; or
R⁶ is —OH, —NHCH₂C(O)OH, or —NH—C(O)OH with a covalent bond to L from the terminal oxygen in any one of —OH, —NHCH₂C(O)OH, or —NH—C(O)OH;
Q is —CH₂— or —O—;
R¹ is —C₁-C₈ alkyl, —C₁-C₈ alkenyl, or —C₁-C₈ alkynyl;
r is three or four; and
k is an integer from one to thirty;
wherein T is not a compound selected from the table below

| ID. | Structure |
|---|---|
| P2 | 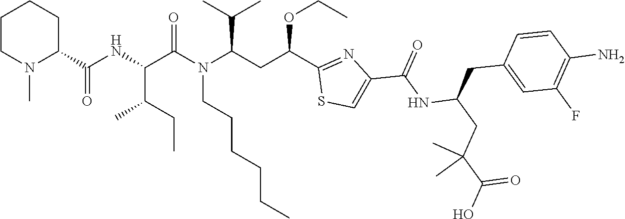 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P5 | 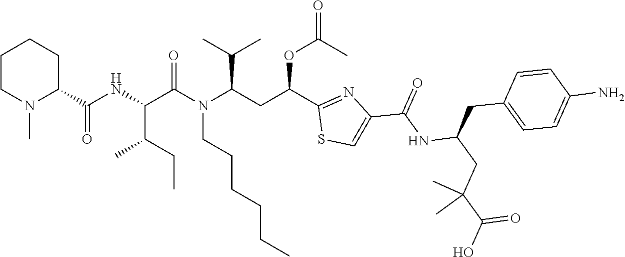 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 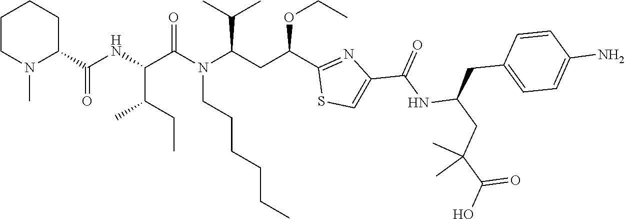 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 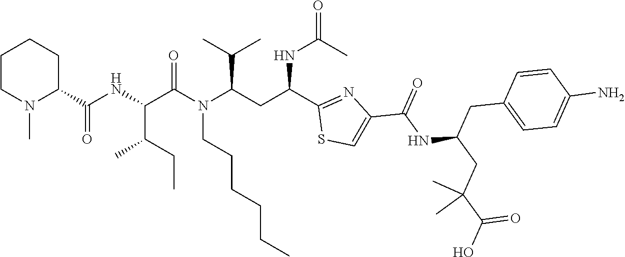 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P10 | 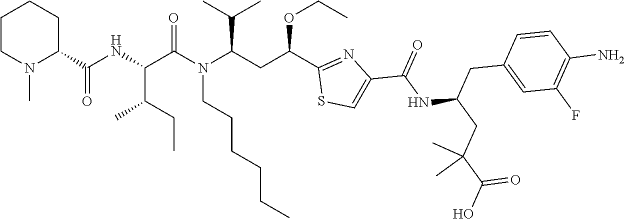 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 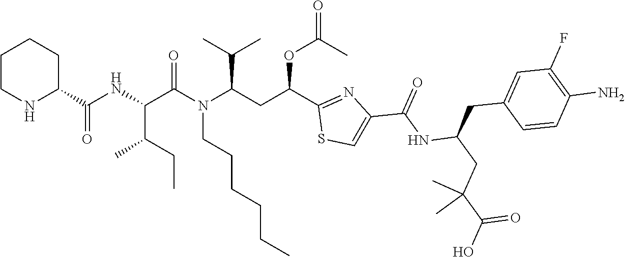 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 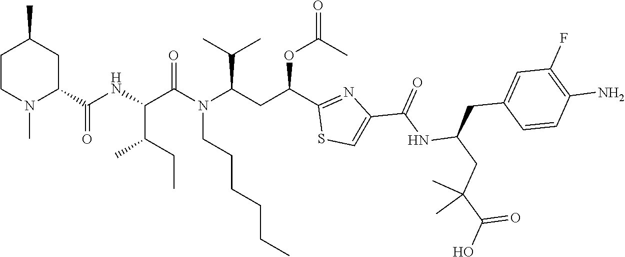 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 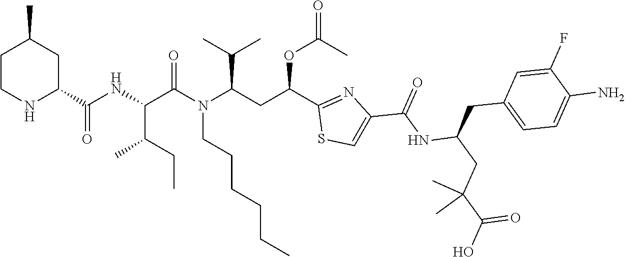 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 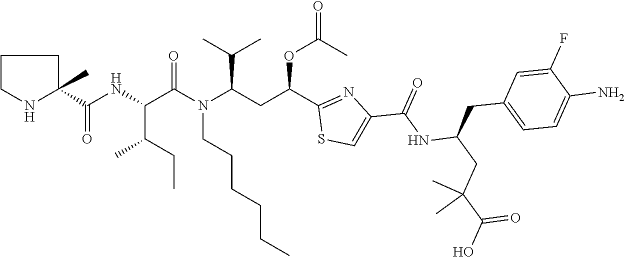 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 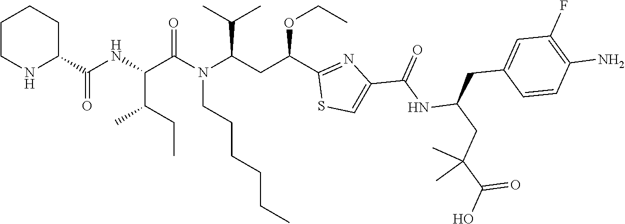 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 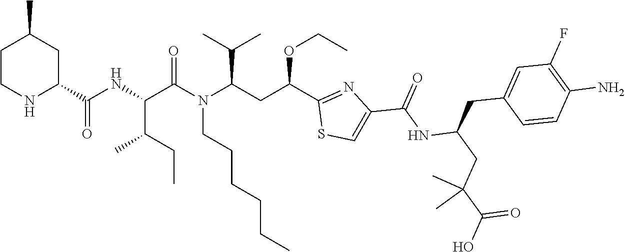 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 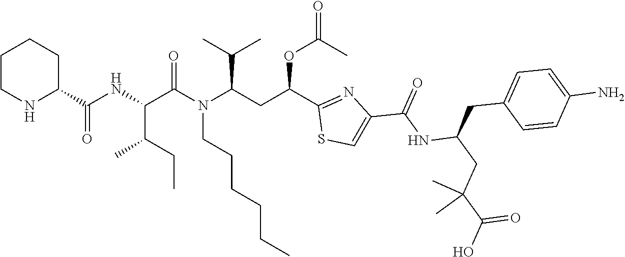 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 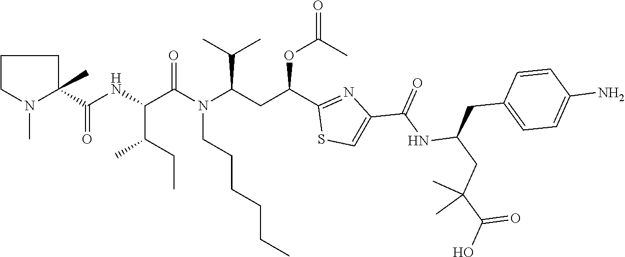 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 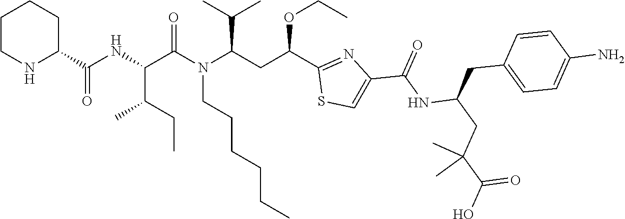 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 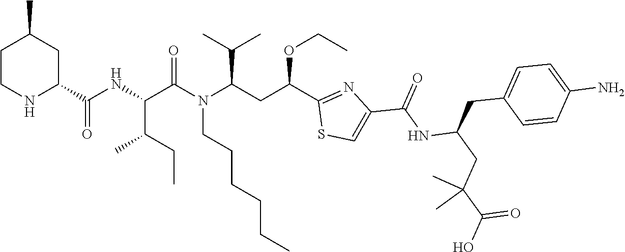 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 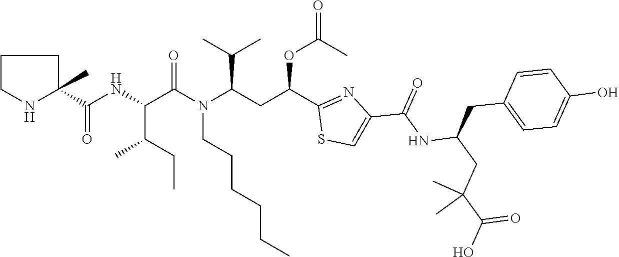 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 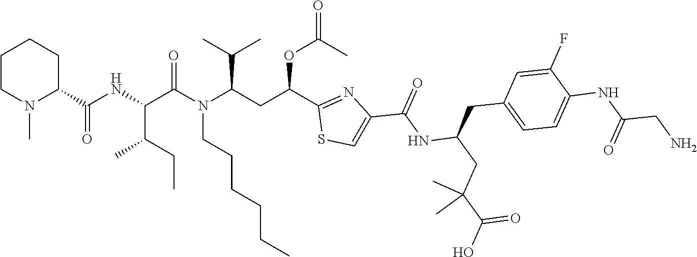 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 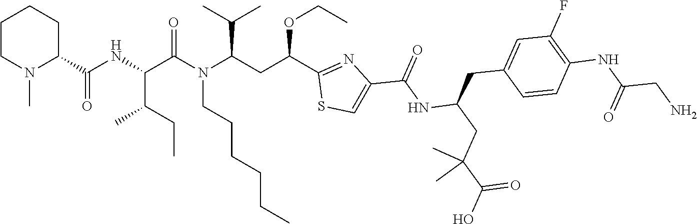 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 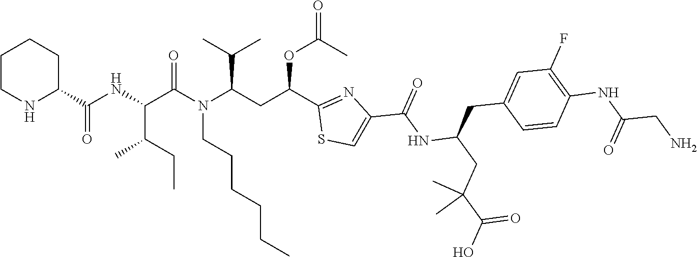 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 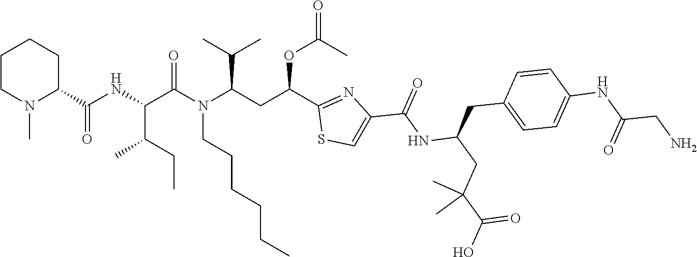 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 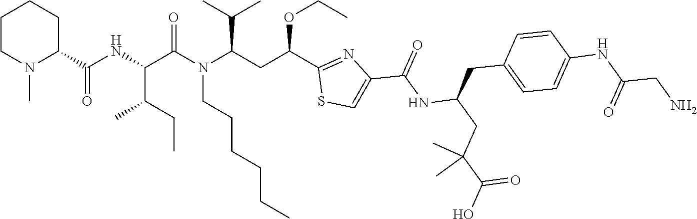 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 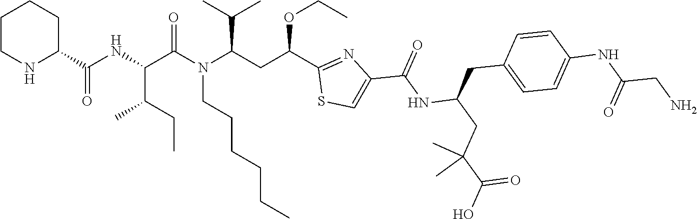 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 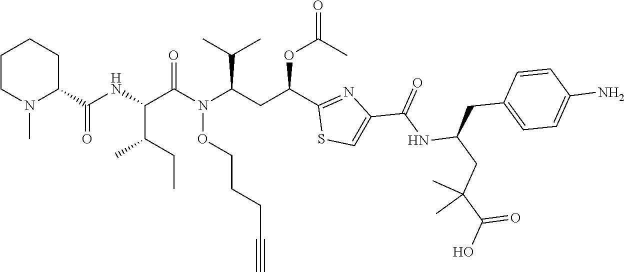 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 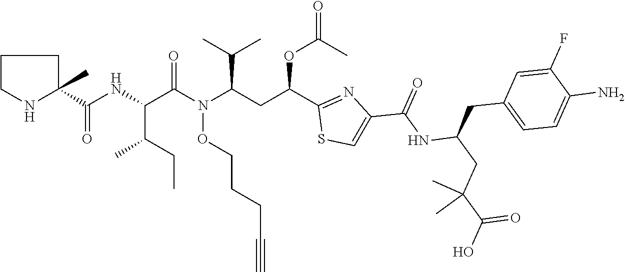 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 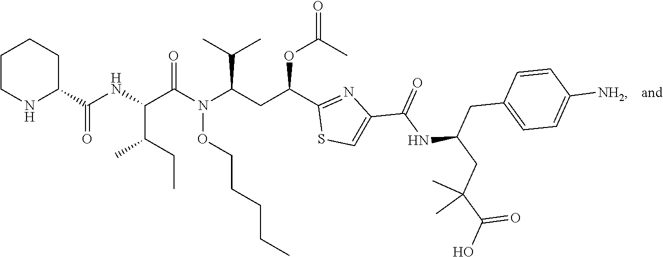 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 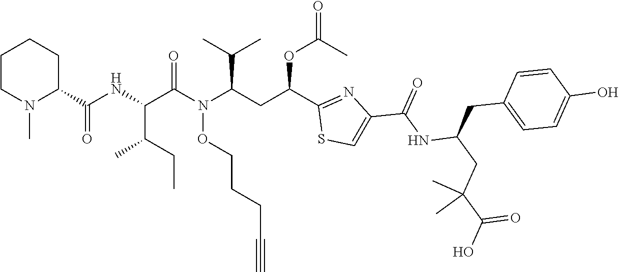 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 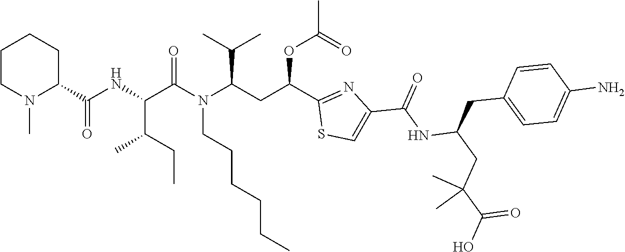 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 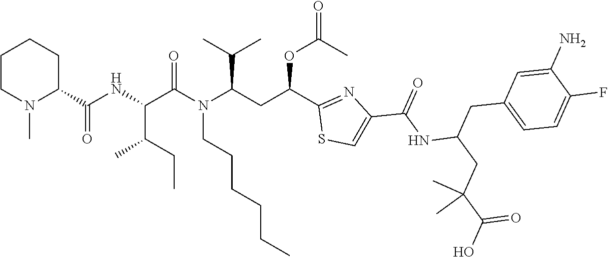 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 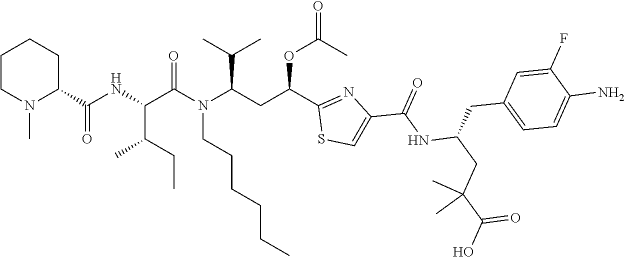 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 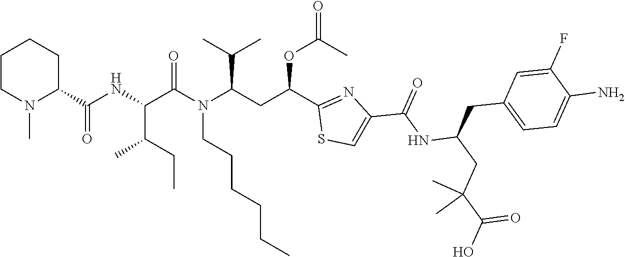 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 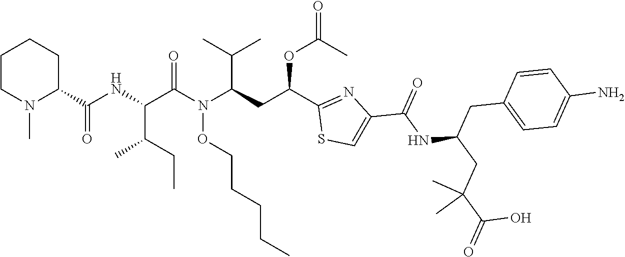 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 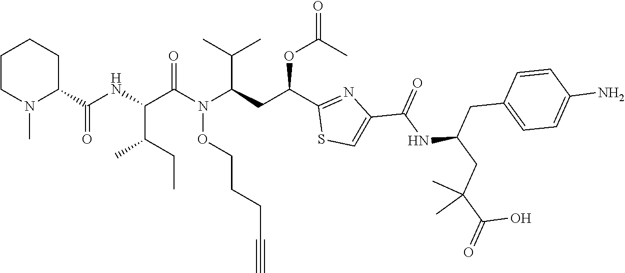 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 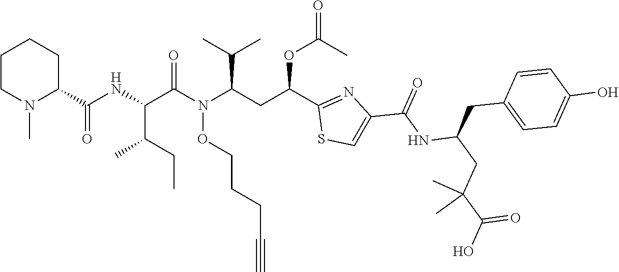 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 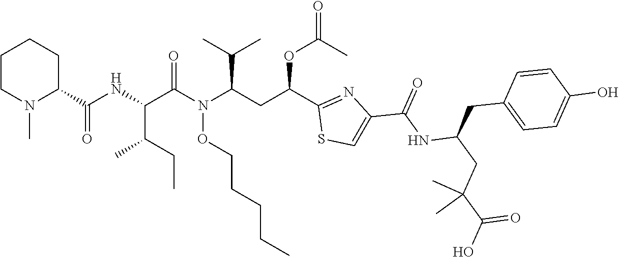 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 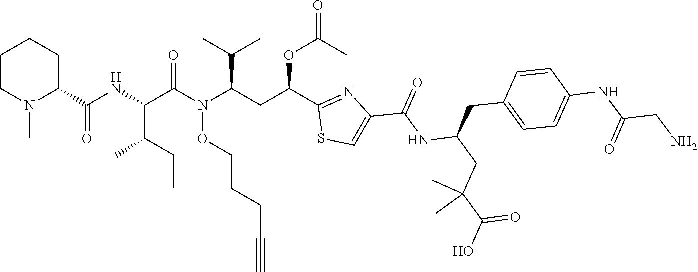 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

2. A compound having the following formula 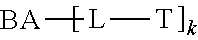
or a pharmaceutically acceptable salt thereof, wherein
BA is a binding agent;
L is a linker covalently bound to BA and to T;
T is 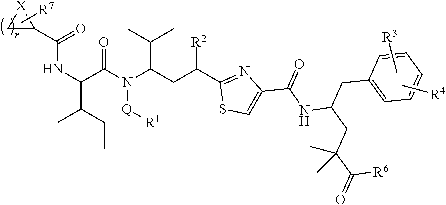
wherein
X is —O— or —NR⁵;
R⁵ is hydrogen, —CH₃—, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, or —C(O)—CH₂—NH₂;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, or —O—C(O)—NH—(CH₂CH₂O)₂—(CH₂)₂OH;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, or —NH—CH₂—C(O)—NH—(CH₂CH₂O)₂—(CH₂)₂NH;
R⁴ is hydrogen or —F;
R⁷ when present is —CH₃;
R⁶ is —OH or —NH—C(O)OH;
Q is —CH₂— or —O—;
R¹ is —C₁-C₈ alkyl, —C₁-C₈ alkenyl, or —C₁-C₈ alkynyl;
r is three or four; and
k is an integer from one to thirty;
wherein T is not a compound selected from the Table below

| ID. | Structure |
|---|---|
| P1 | 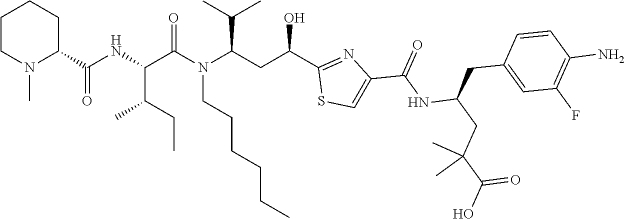 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P2 | 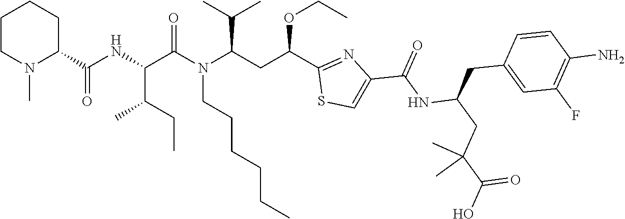 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P3 | 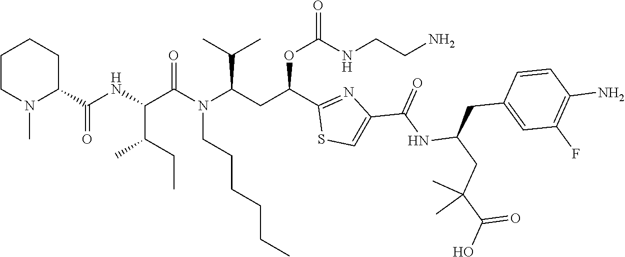 [SMILES: CCCCCCN(C(=O)[C@@H](NC(=O)[C@H]1CCCCN1C)[C@@H](C)CC)[C@H](C[C@@H](OC(=O)NCCN)c1nc(C(=O)N[C@@H](Cc2ccc(N)c(F)c2)CC(C)(C)C(=O)O)cs1)C(C)C] |
| P4 | 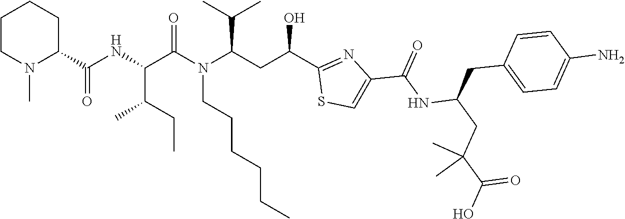 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)cc2)CC(C)(C)C(=O)O)cs1)C(C)C] |
| P5 | 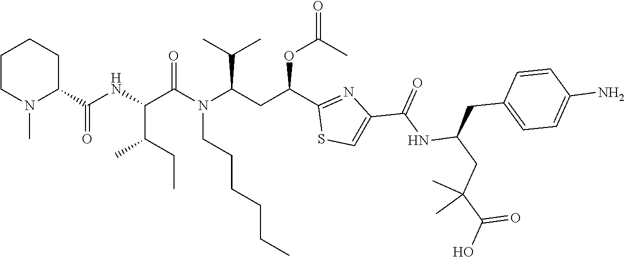 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 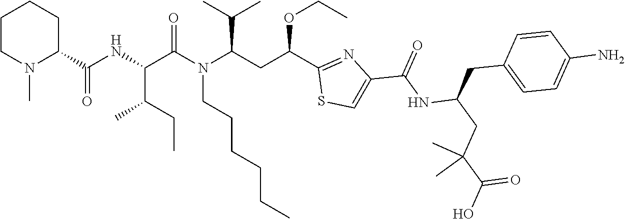 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 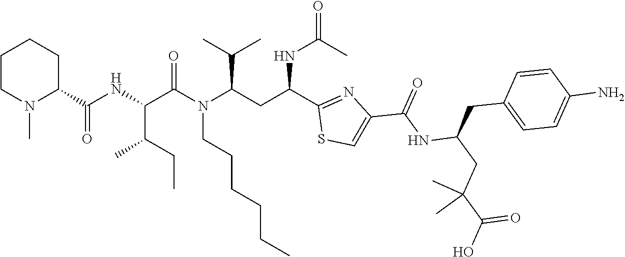 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P8 | 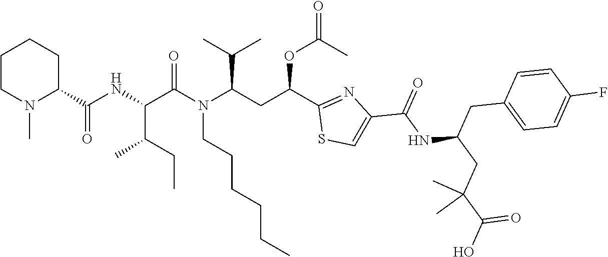 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(F)cc2)CC(C)(C)C(=O)O)cs1)C(C)C] |
| P9 | 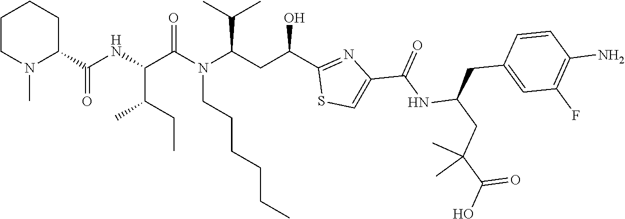 [SMILES: CCCCCCN(C(=O)[C@@H](NC(=O)[C@H]1CCCCN1C)[C@@H](C)CC)[C@H](C[C@@H](O)c1nc(C(=O)N[C@@H](Cc2ccc(N)c(F)c2)CC(C)(C)C(=O)O)cs1)C(C)C] |
| P10 | 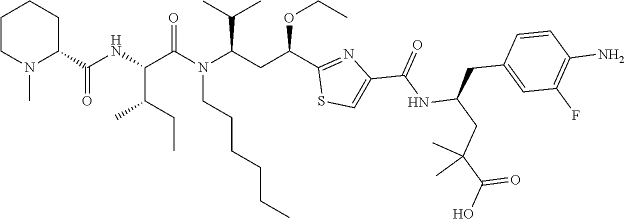 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 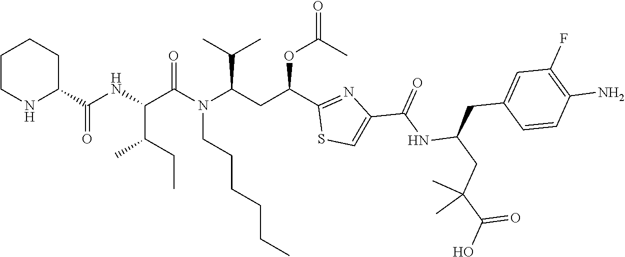 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 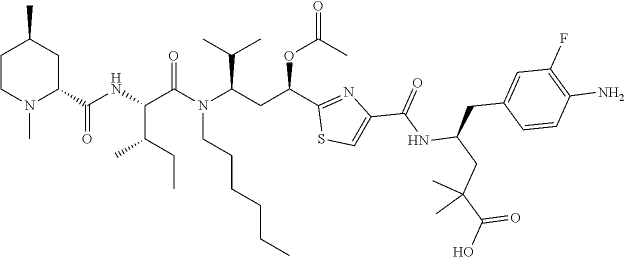 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 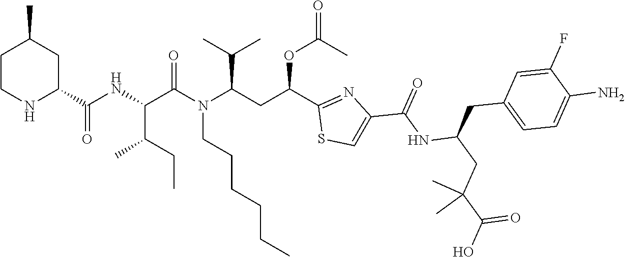 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 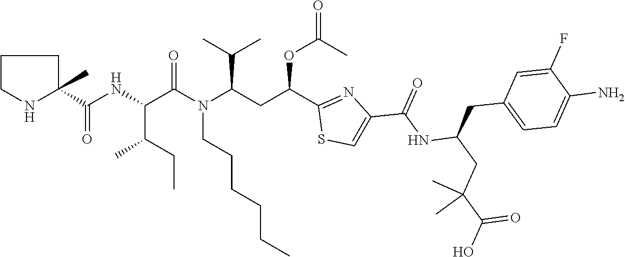 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 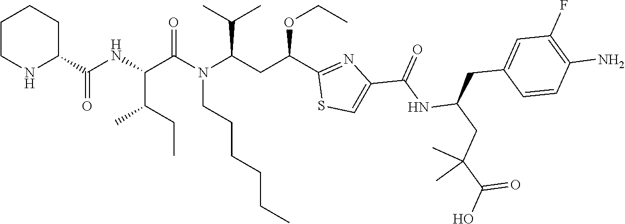 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 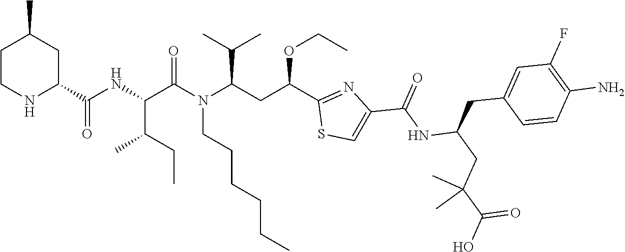 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 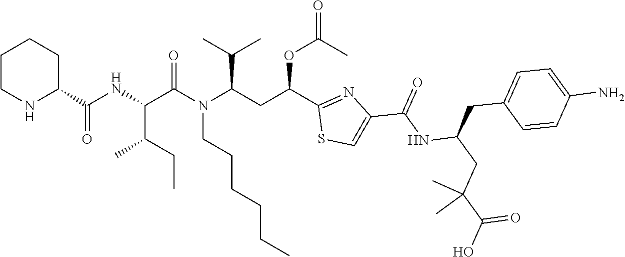 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 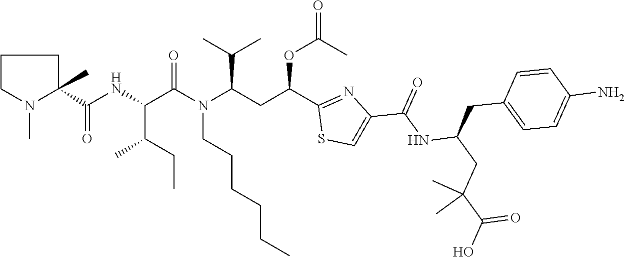 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 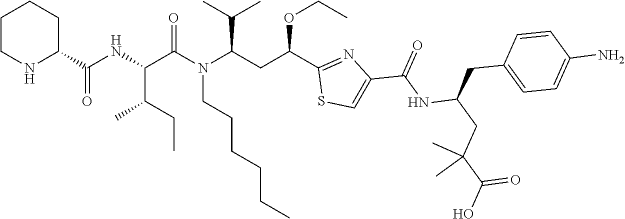 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 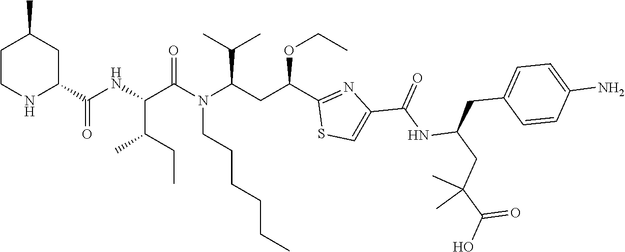 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 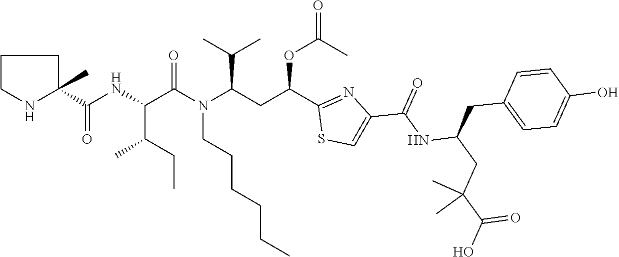 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 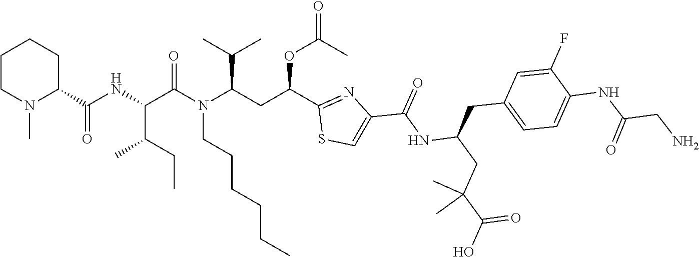 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 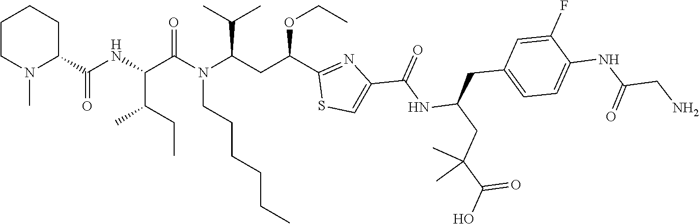 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 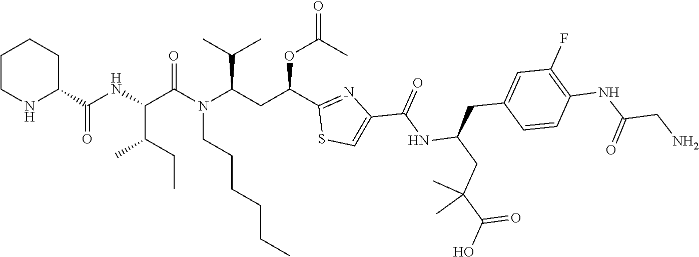 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 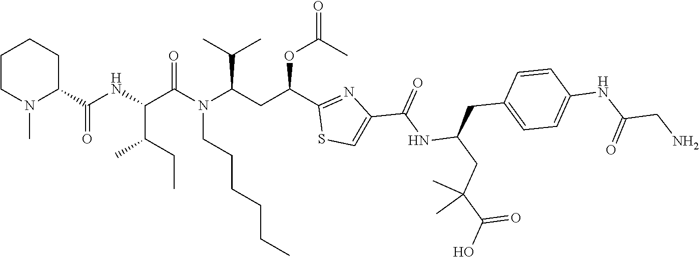 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 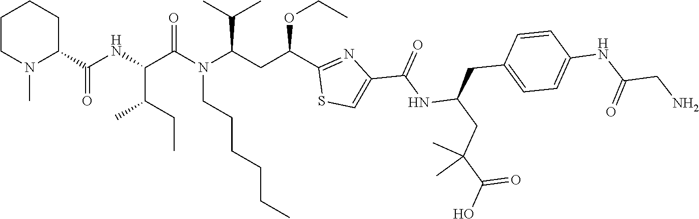 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 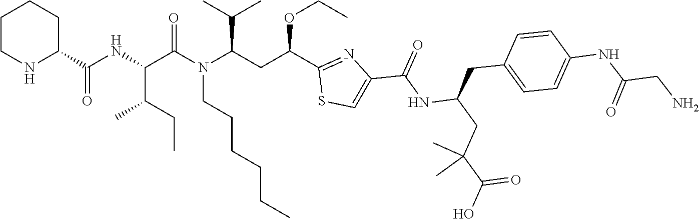 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 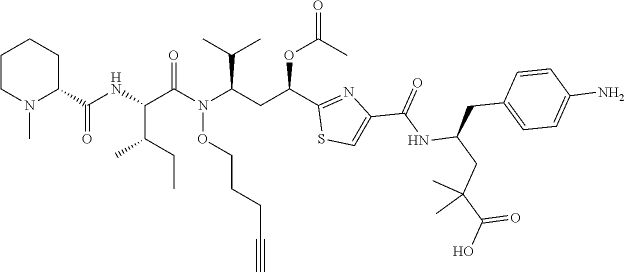 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 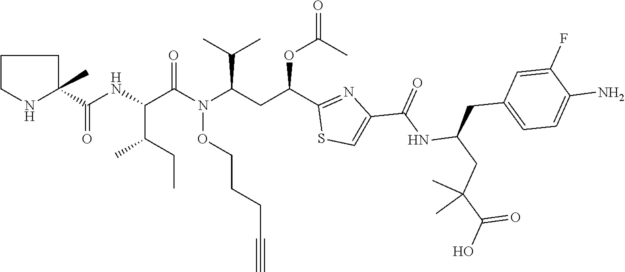 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 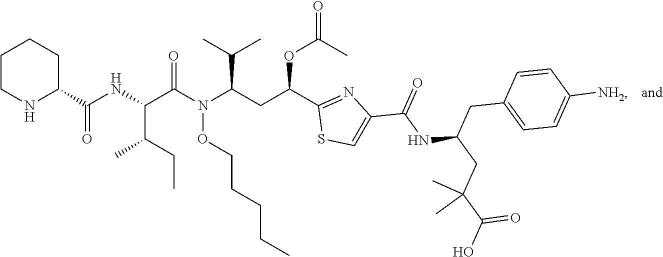 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 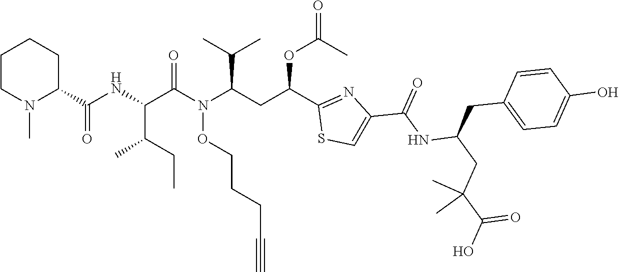 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 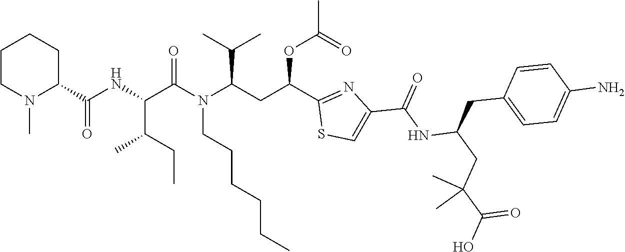 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 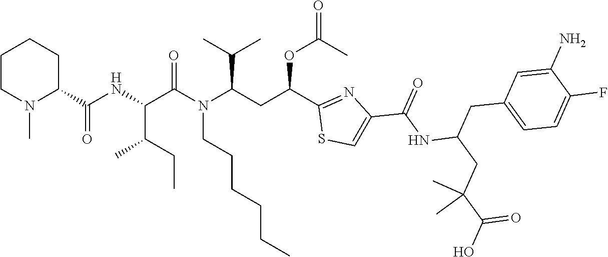 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 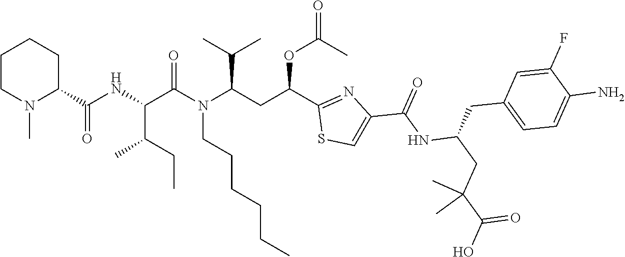 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 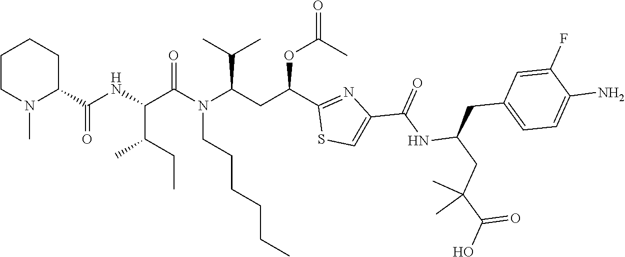 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 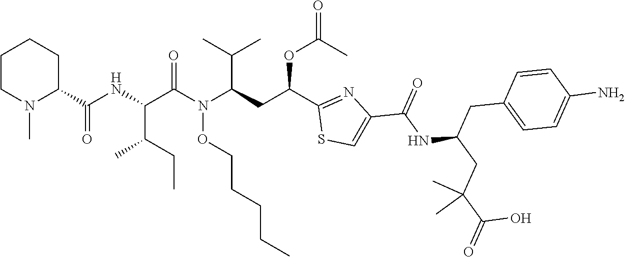 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 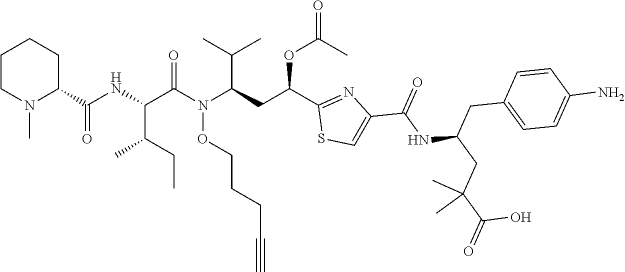 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 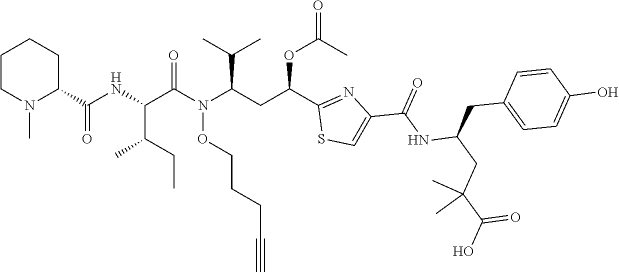 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 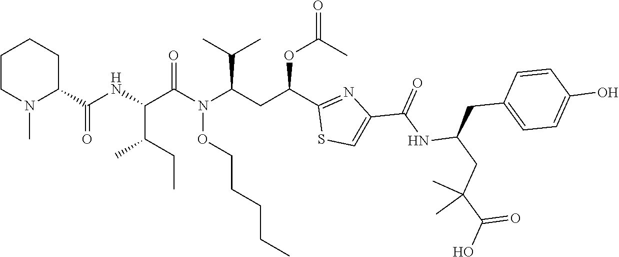 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| D-5a | 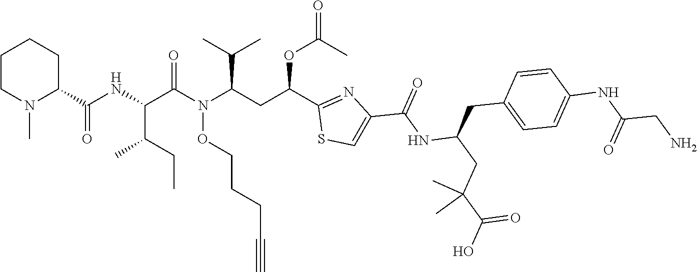 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

or a pharmaceutically acceptable salt thereof, covalently bound to L.

3. The compound of claim 1, having a Formula A, B, C, D, or E 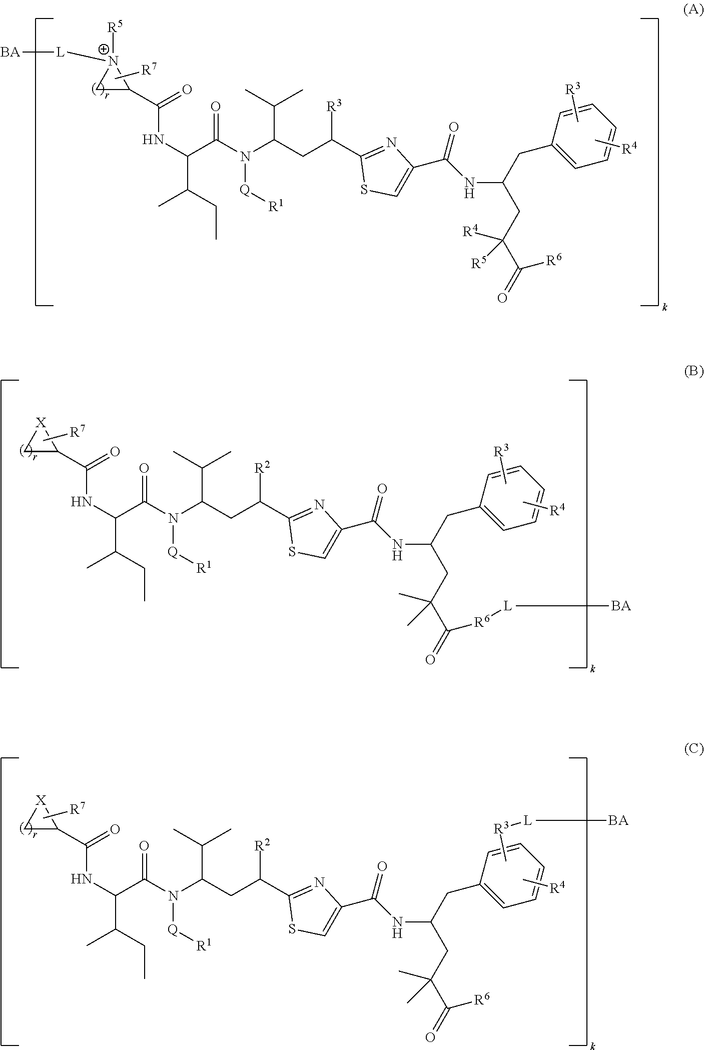 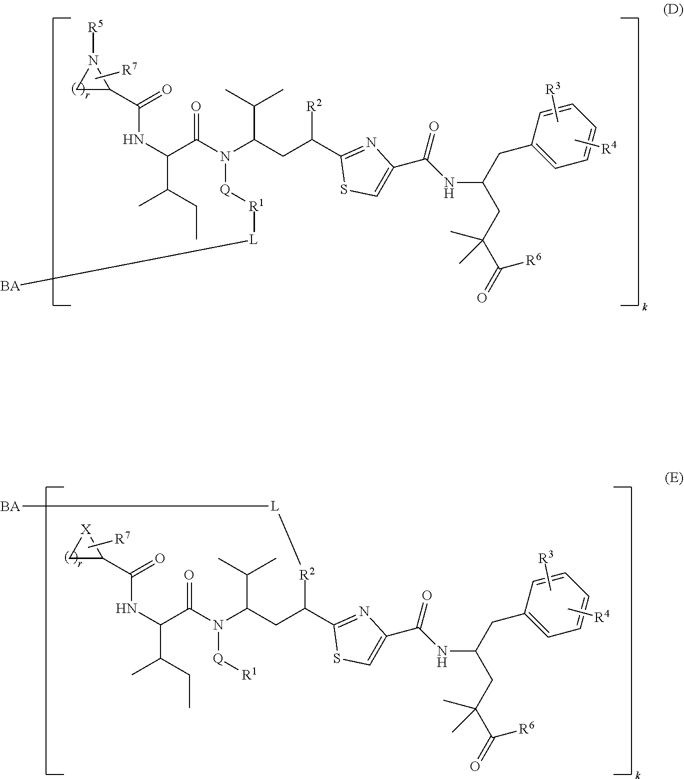
wherein L is a linker.

4. The compound of claim 3, wherein the compound is of the Formula A′, B′, C′, D′, or E′ 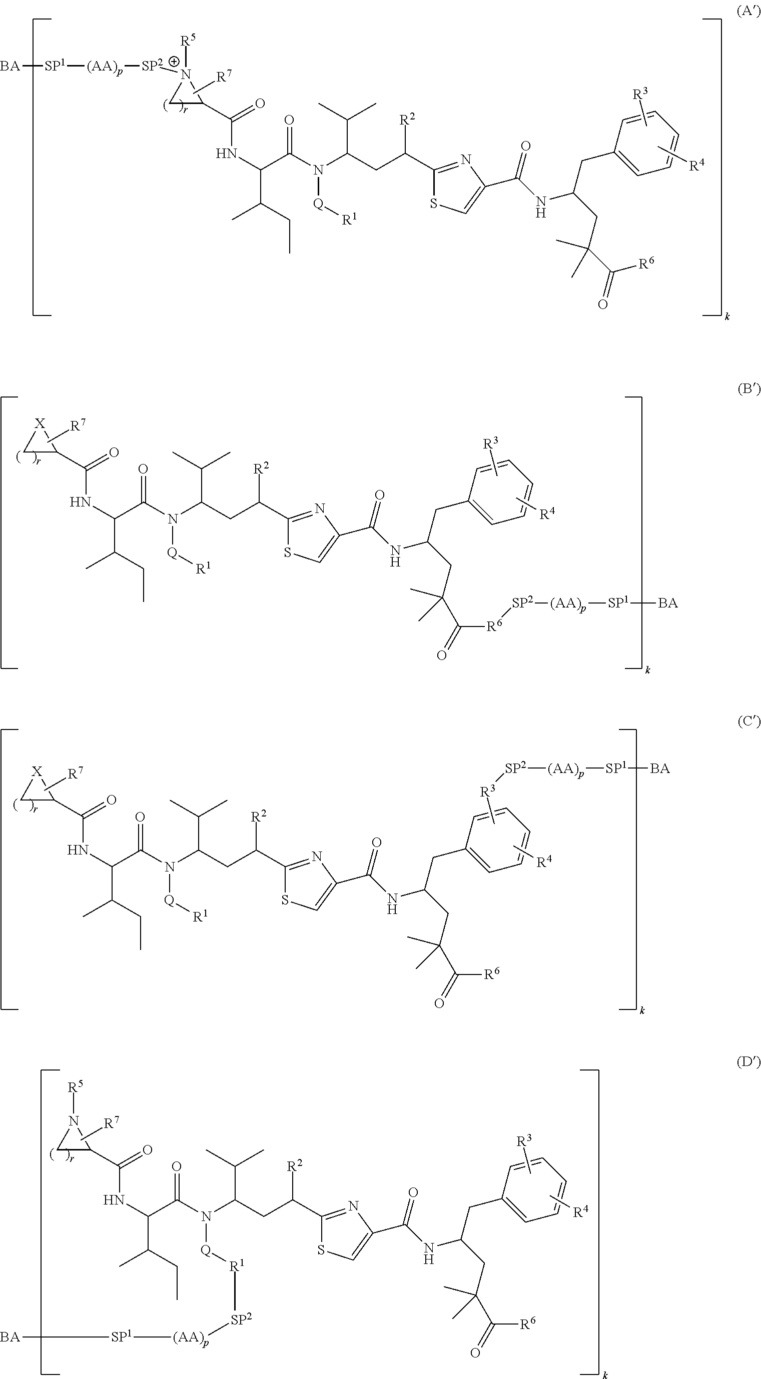 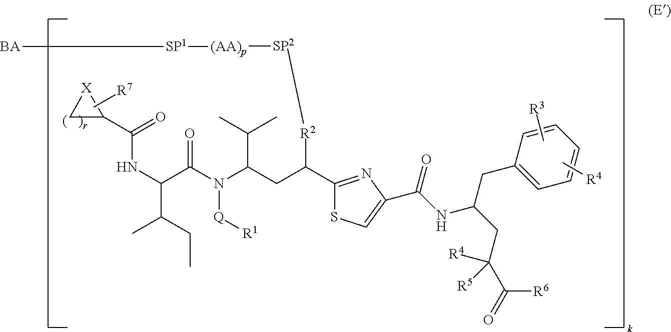
wherein SP¹ and SP², when present, are spacer groups;
each AA, when present, is a second amino acid residue; and
p is an integer from zero to ten.

5. The compound of claim 4, wherein
the —SP²— spacer, when present, is 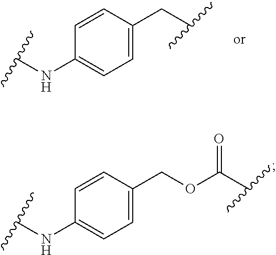 [SMILES: *Cc1ccc(N*)cc1 ; *Nc1ccc(COC(*)=O)cc1]
the second -(AA)ₚ- is 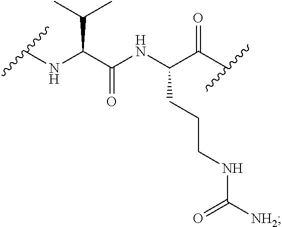 [SMILES: *N[C@H](C(=O)N[C@@H](CCCNC(N)=O)C(*)=O)C(C)C]
the —SP¹— spacer is 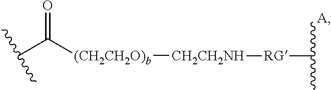
wherein RG′ is a reactive group residue following reaction of a reactive group RG with a binding agent; 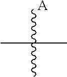
is a bond, direct or indirect, to the binding agent; and
b is an integer from one to four.

6. The compound of claim 5, wherein the binding agent is an antibody modified with a primary amine compound according to the Formula H₂N-LL-X, wherein LL is a divalent linker selected from the group consisting of a divalent polyethylene glycol (PEG) group;
—(CH₂)ₙ—;
—(CH₂CH₂O)ₙ—(CH₂)ₚ—;
—(CH₂)ₙ—N(H)C(O)—(CH₂)ₘ—;
—(CH₂CH₂O)ₙ—N(H)C(O)—(CH₂CH₂O)ₘ—(CH₂)ₚ—;
—(CH₂)ₙ—C(O)N(H)—(CH₂)ₘ—;
—(CH₂CH₂O)ₙ—C(O)N(H)—(CH₂CH₂O)ₘ—(CH₂)ₚ—;
—(CH₂)ₙ—N(H)C(O)—(CH₂CH₂O)ₘ—(CH₂)ₚ—;
—(CH₂CH₂O)ₙ—N(H)C(O)—(CH₂)ₘ—;
—(CH₂)ₙ—C(O)N(H)—(CH₂CH₂O)ₘ—(CH₂)ₚ—; and
—(CH₂CH₂O)ₙ—C(O)N(H)—(CH₂)ₘ—,
wherein
n is an integer selected from one to twelve;
m is an integer selected from zero to twelve;
p is an integer selected from zero to two; and
X is selected from the group consisting of —SH, —N₃, —C≡CH, —C(O)H, tetrazole,

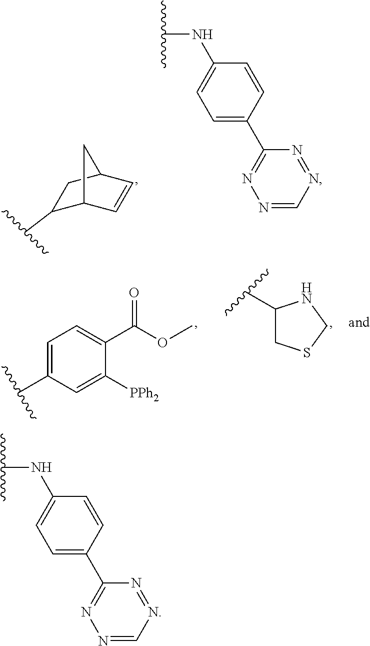

7. The compound of claim 6, wherein the binding agent is an antibody modified with a primary amine according to the following formula

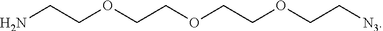 [SMILES: [N-]=[N+]=NCCOCCOCCOCCN]

8. The compound of claim 4, wherein Q is —O—.

9. The compound of claim 4, wherein
Q is —CH₂—;
X is —NR⁵,
R⁵ is —CH₃ or —(CH₂)₂—OH;
R¹ is —C₅ alkyl;
R⁶ is —OH;
R⁷ is —CH₃; and
r is four.

10. The compound of claim 9, according to the structure of C′, or a pharmaceutically acceptable salt thereof.

11. The compound of claim 10, wherein R³ is —NH—(CH₂)₂O—, —NH—CH₂—C(O)—NH—, —NH—C(O)—CH₂NH—, or —NH—CH₂—C(O)—NH—(CH₂CH₂O)₂—(CH₂)₂NH—; and R⁴ is hydrogen.

12. The compound of claim 10, wherein R³ is —NH—(CH₂)₂OH.

13. The compound of claim 10, wherein R³ is —NH—(CH₂)₂O—.

14. The compound of claim 4, wherein
Q is —CH₂—;
X is —NR⁵;
R¹ is —C₅ alkyl;
R⁶ is —OH;
R⁷ when present is —CH₃; and
r is four.

15. The compound of claim 14, according to the structure of A′, or a pharmaceutically acceptable salt thereof.

16. The compound of claim 15, wherein R⁵ is —C(O)—CH₂—NH₂.

17. The compound of claim 4, wherein
Q is —CH₂—;
X is —NR⁵,
R⁵ is —CH₃;
R¹ is —C₅ alkyl;
R⁶ is —OH;
R⁷ when present is —CH₃; and
r is four.

18. The compound of claim 17, according to the structure of E′, or a pharmaceutically acceptable salt thereof.

19. The compound of claim 18, wherein R² is —O—C(O)—NH—(CH₂CH₂O)₂—(CH₂)₂O—.

20. The compound of claim 4, wherein
Q is —CH₂—;
X is —NR⁵
R⁵ is —CH₃ or —(CH₂)₂—OH;
R¹ is —C₅ alkyl;
R⁶ is —OH;
R⁷ when present is —CH₃; and
r is four.

21. The compound of claim 20, according to the structure of C′, or a pharmaceutically acceptable salt thereof.

22. The compound of claim 21, wherein R³ is —NH—CH₂—C(O)—NH— and R⁴ is hydrogen.

23. The compound of claim 4, wherein
Q is —CH₂—;
X is —NR⁵
R⁵ is —CH₃ or —(CH₂)₂—OH;
R¹ is —C₅ alkyl;
R⁶ is —OH;
R⁷ when present is —CH₃; and
r is four.

24. The compound of claim 23, according to the structure of C′, or a pharmaceutically acceptable salt thereof.

25. The compound of claim 24, wherein R³ is —NH—C(O)—CH₂NH— and R⁴ is hydrogen.

26. The compound of claim 24, wherein R³ is —NH—CH₂—C(O)—NH—(CH₂CH₂O)₂—(CH₂)₂NH— and R⁴ is hydrogen.

27. The compound of claim 4, selected from the group consisting of 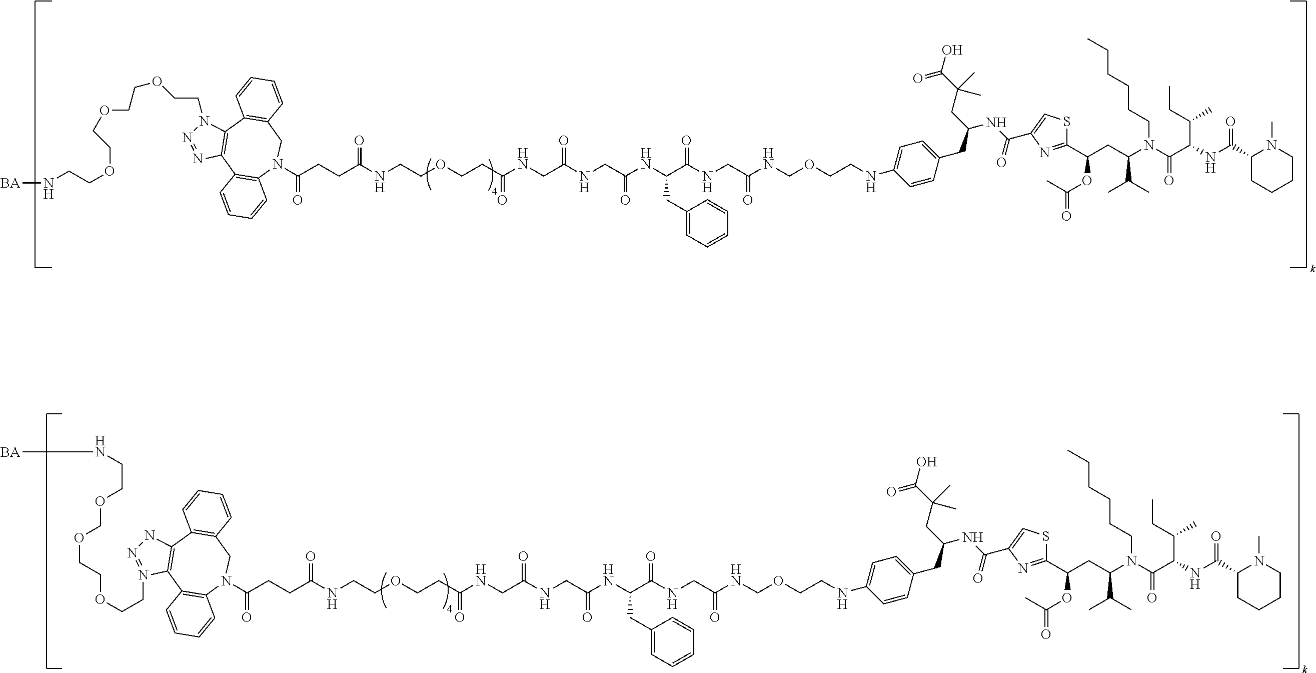 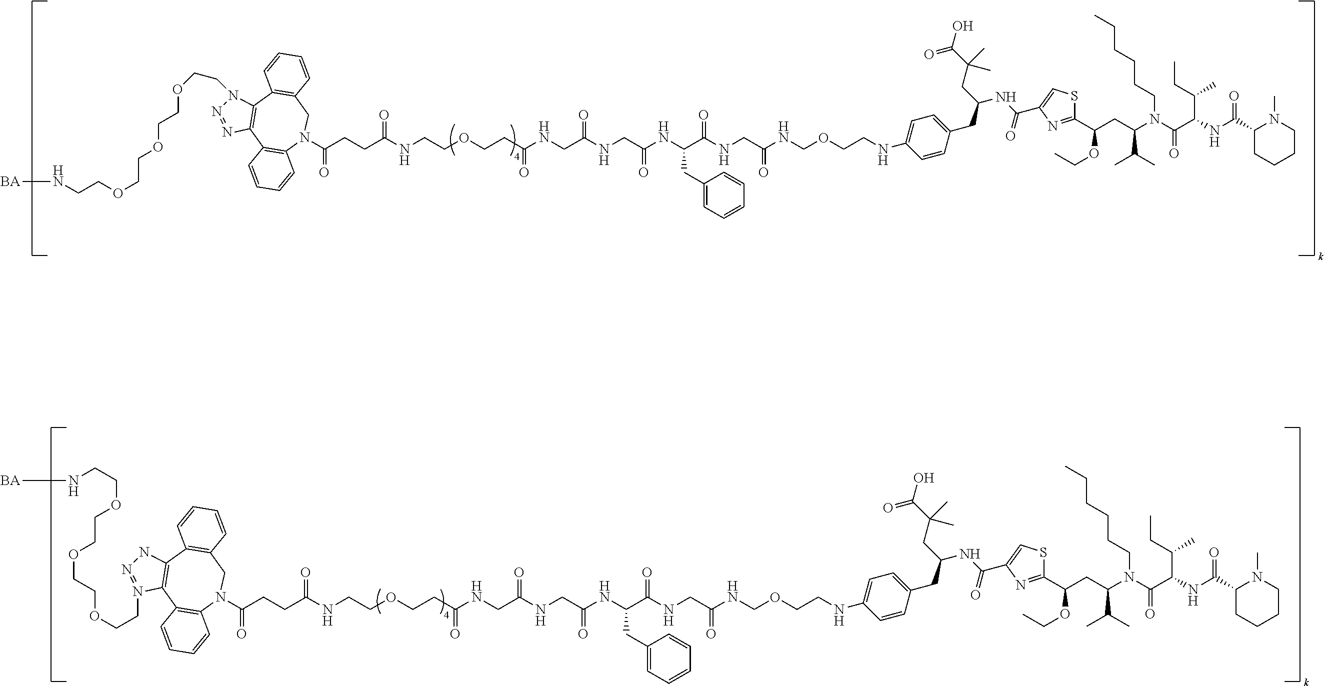 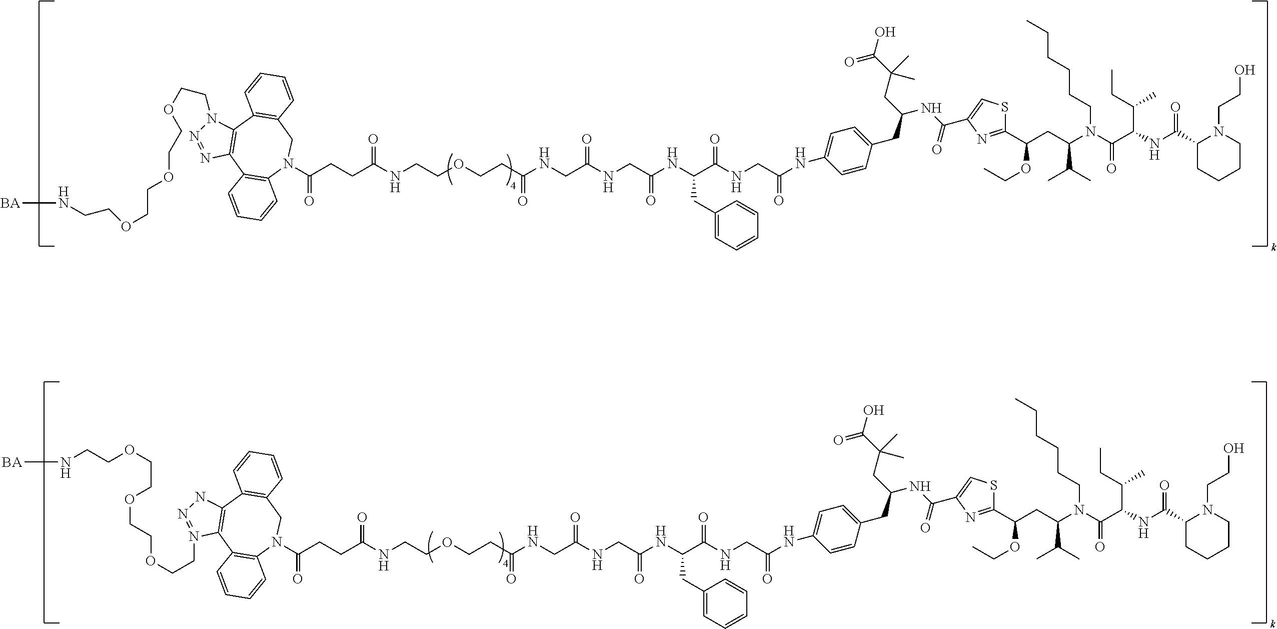 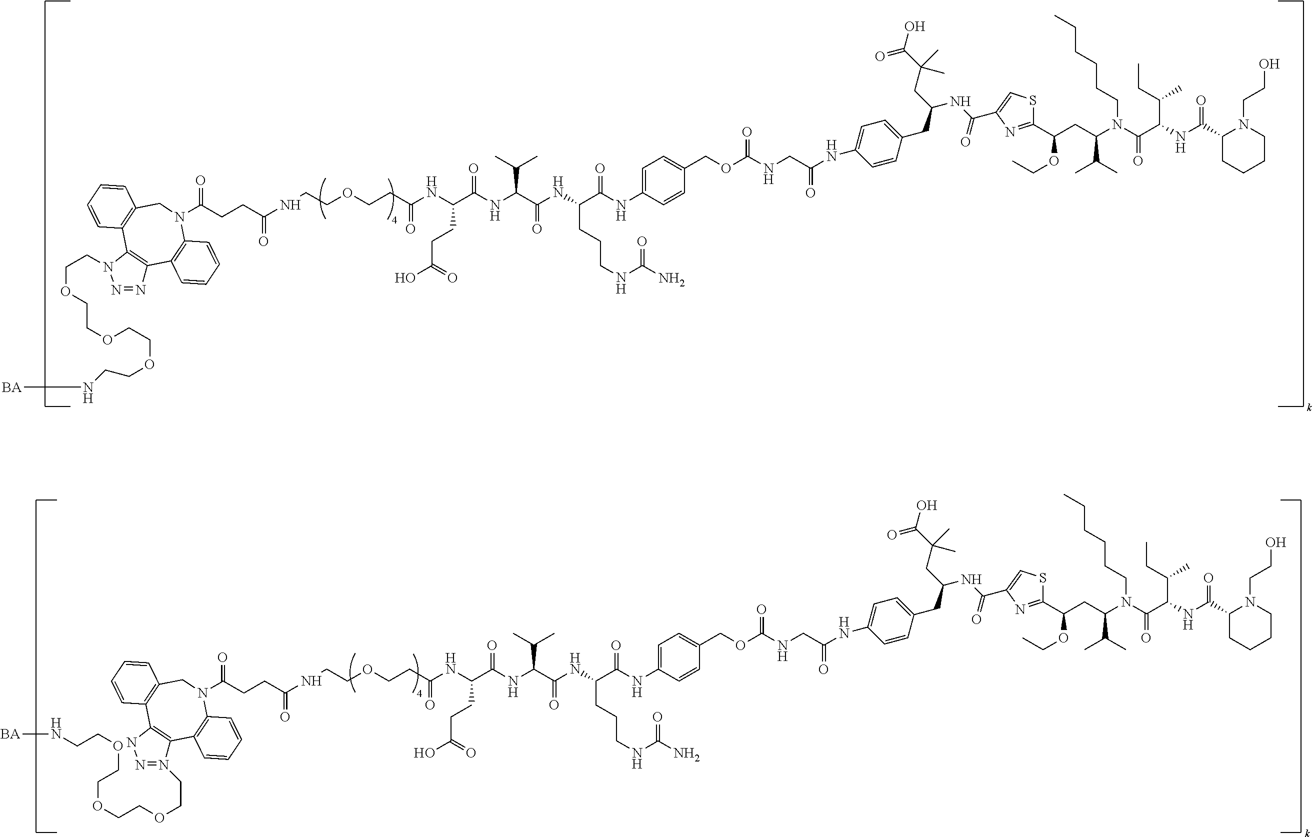 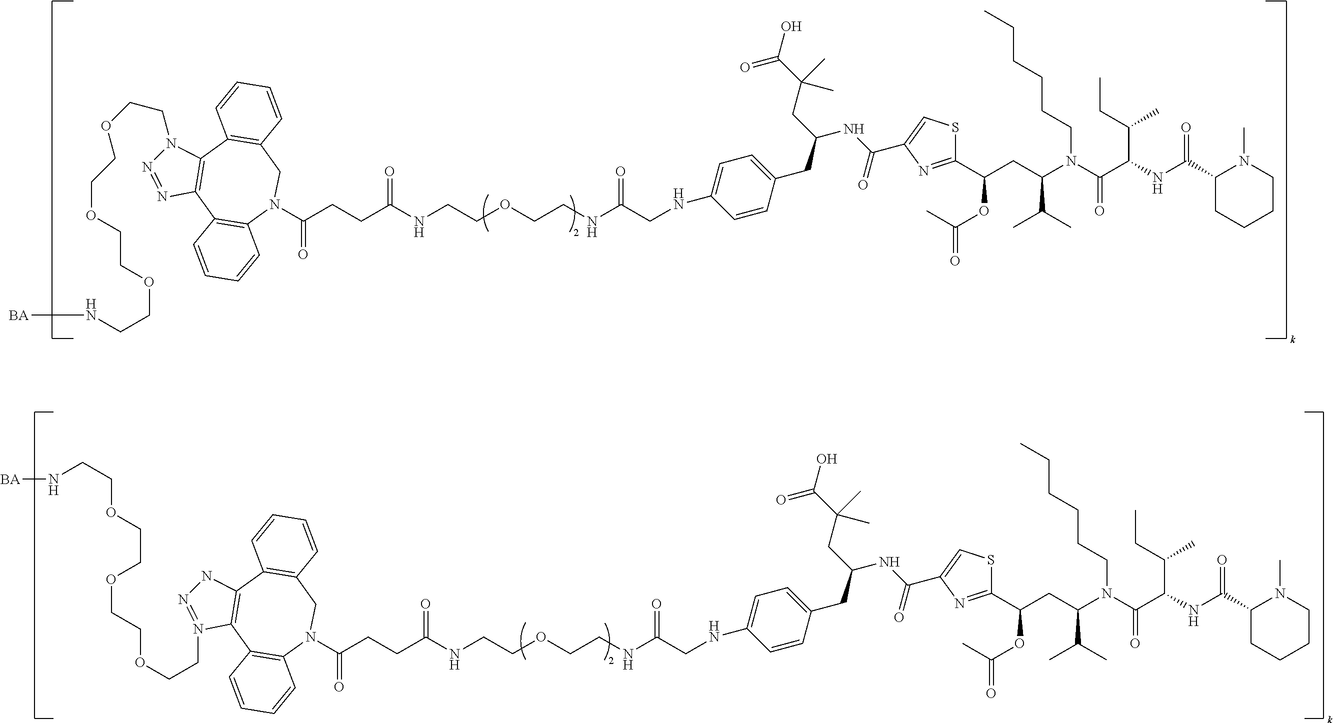 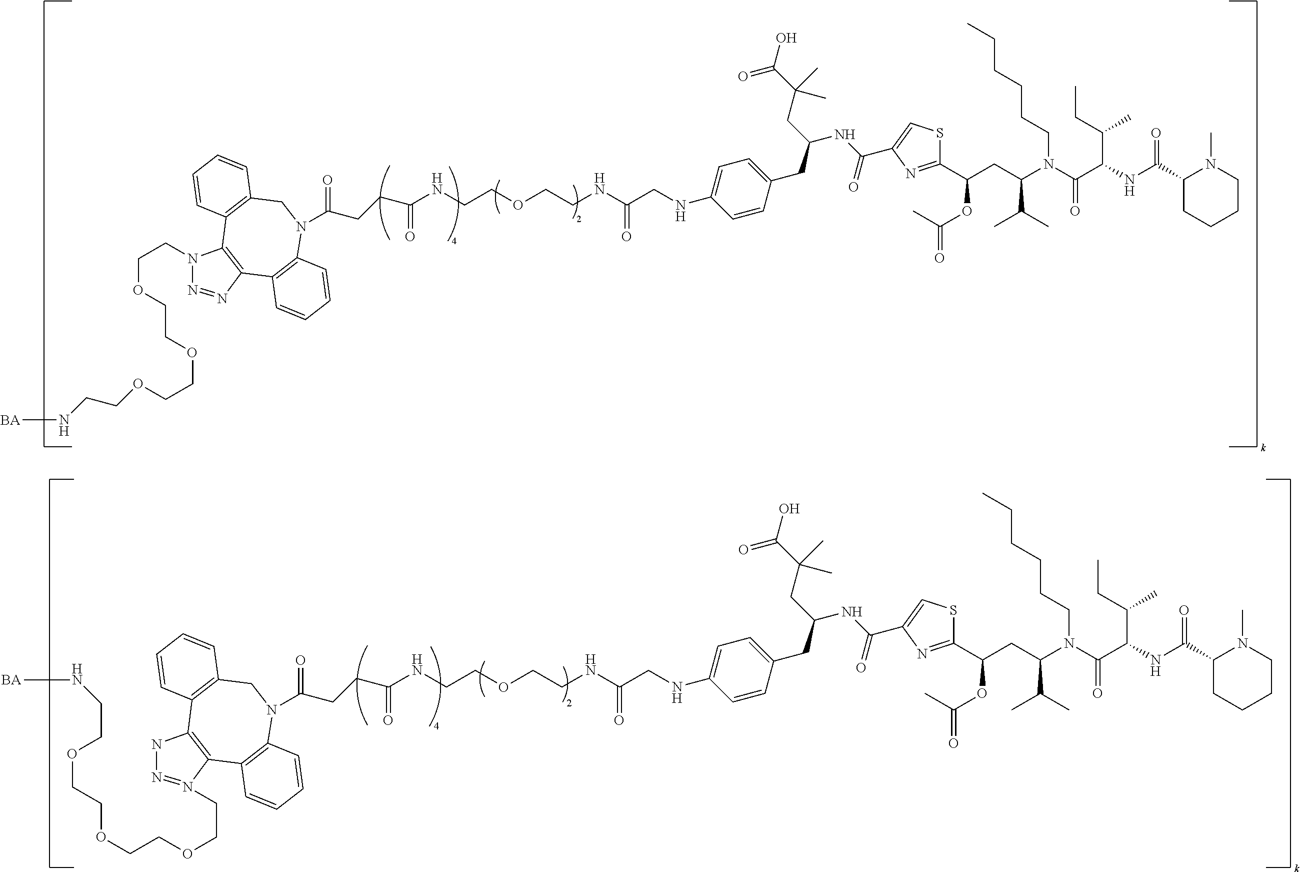 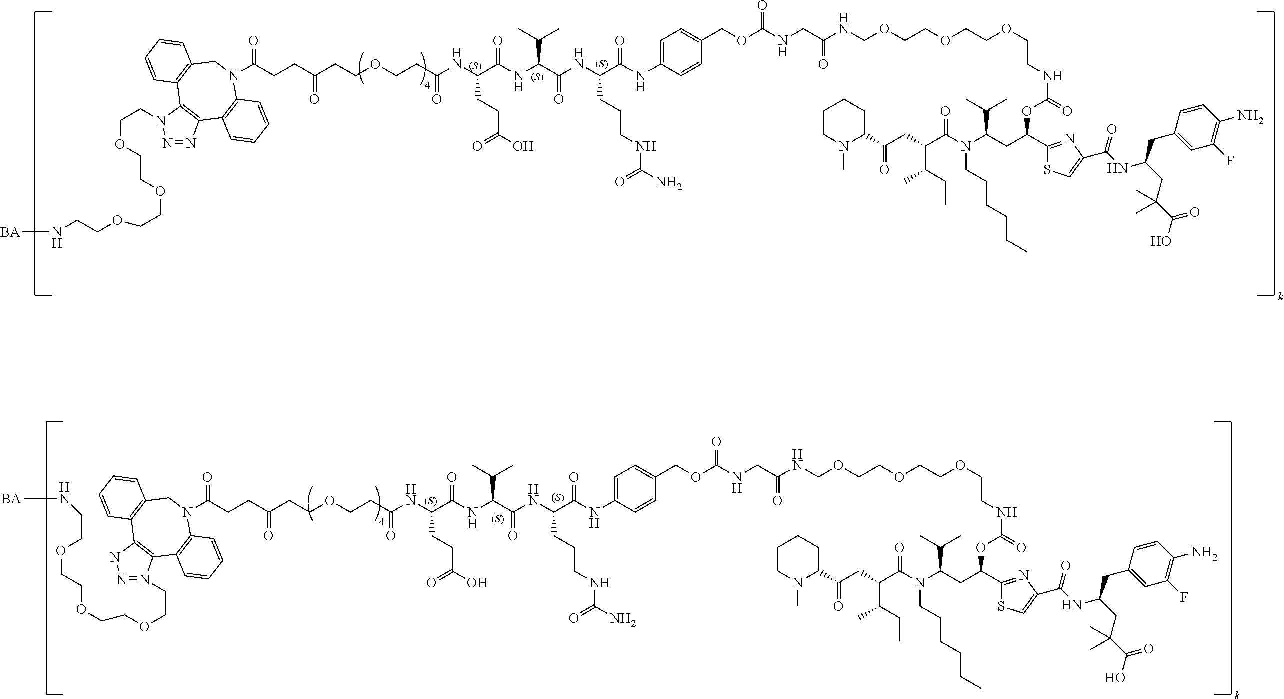 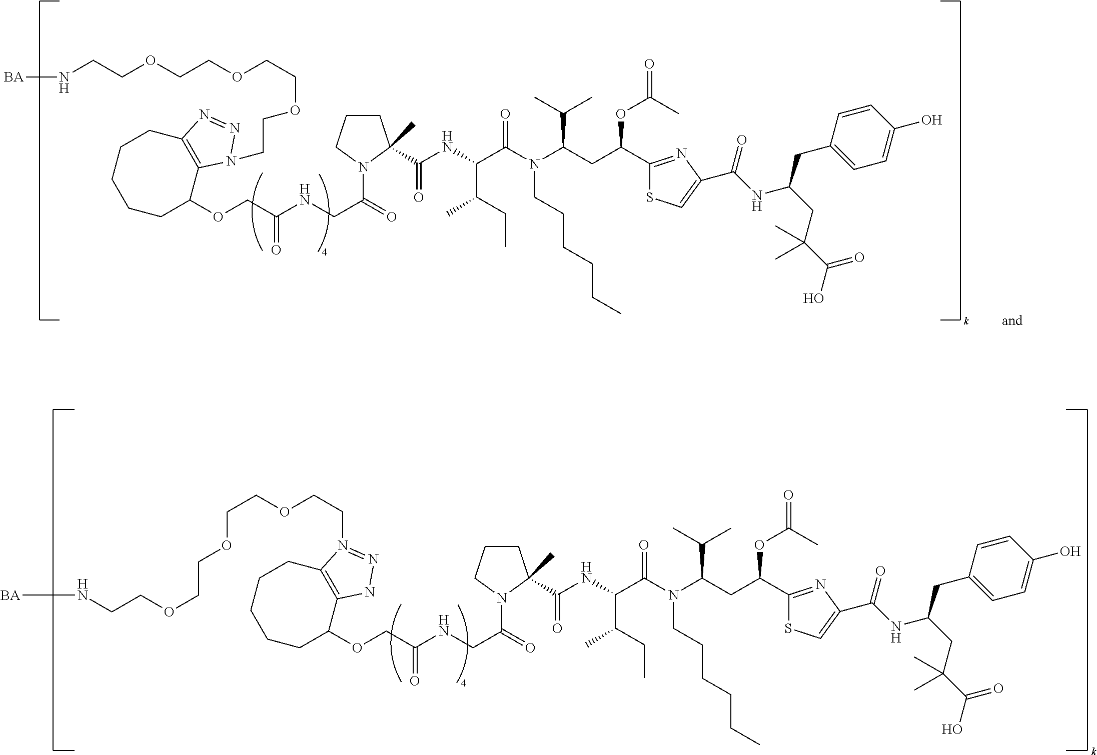
or a pharmaceutically acceptable salt thereof,
wherein BA is a binding agent; and k is one, two, three, or four.

28. The compound of claim 27, wherein BA is an antibody or antigen-binding fragment thereof.

29. The compound of claim 28, wherein BA is a transglutaminase-modified antibody or antigen-binding fragment thereof comprising at least one glutamine residue used for conjugation.

30. The compound of claim 28, wherein BA is a transglutaminase-modified antibody or antigen-binding fragment thereof comprising at least two glutamine residues used for conjugation.

31. The compound of claim 28, wherein BA is a transglutaminase-modified antibody or antigen-binding fragment thereof comprising at least four glutamine residues used for conjugation.

32. The compound of claim 30, wherein BA is a transglutaminase-modified antibody or antigen-binding fragment thereof wherein conjugation is at two Q295 residues; and k is two.

33. The compound of claim 30, wherein BA is a transglutaminase-modified antibody or antigen-binding fragment thereof wherein conjugation is at two Q295 residues and two N297Q residues; and k is four.

34. The compound of claim 1, wherein the compound is an antibody-drug conjugate comprising an antibody or antigen-binding fragment thereof conjugated to a compound selected from the group consisting of

| ID No./ | |
|---|---|
| Com- | |
| pound | |
| No. | Structure |
| PA2′ | 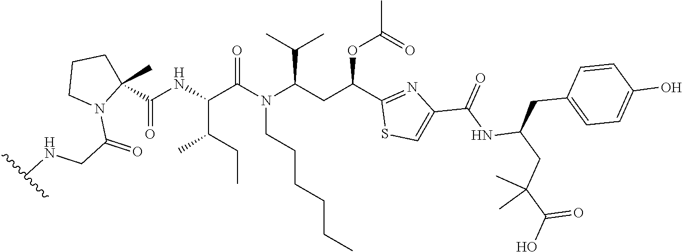 [SMILES: *NCC(=O)N1CCC[C@]1(C)C(=O)N[C@H](C(=O)N(CCCCCC)[C@H](C[C@@H](OC(C)=O)c1nc(C(=O)N[C@@H](Cc2ccc(O)cc2)CC(C)(C)C(=O)O)cs1)C(C)C)[C@@H](C)CC] |
| PA13′ | 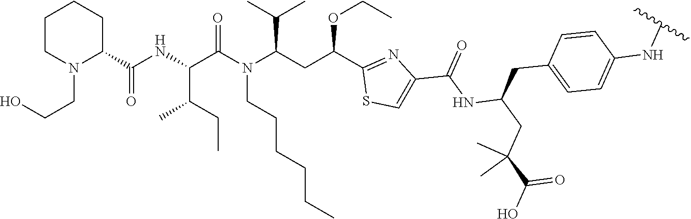 |
| PA14′ | 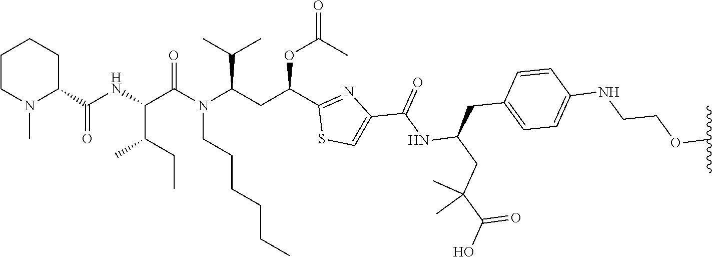 [SMILES: *OCCNc1ccc(C[C@@H](CC(C)(C)C(=O)O)NC(=O)c2csc([C@@H](C[C@H](C(C)C)N(CCCCCC)C(=O)[C@@H](NC(=O)[C@H]3CCCCN3C)[C@@H](C)CC)OC(C)=O)n2)cc1] |
| PA15′ | 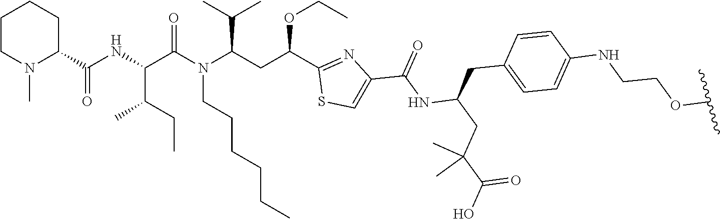 [SMILES: *OCCNc1ccc(C[C@@H](CC(C)(C)C(=O)O)NC(=O)c2csc([C@@H](C[C@H](C(C)C)N(CCCCCC)C(=O)[C@@H](NC(=O)[C@H]3CCCCN3C)[C@@H](C)CC)OCC)n2)cc1] |
| PA20′ | 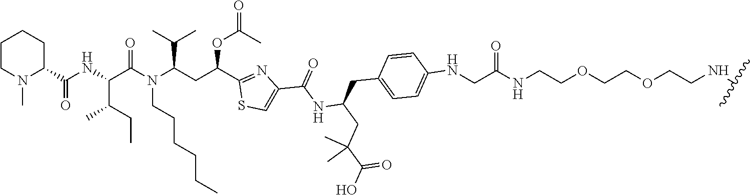 [SMILES: *NCCOCCOCCNC(=O)CNc1ccc(C[C@@H](CC(C)(C)C(=O)O)NC(=O)c2csc([C@@H](C[C@H](C(C)C)N(CCCCCC)C(=O)[C@@H](NC(=O)[C@H]3CCCCN3C)[C@@H](C)CC)OC(C)=O)n2)cc1] |
| PA25′ | 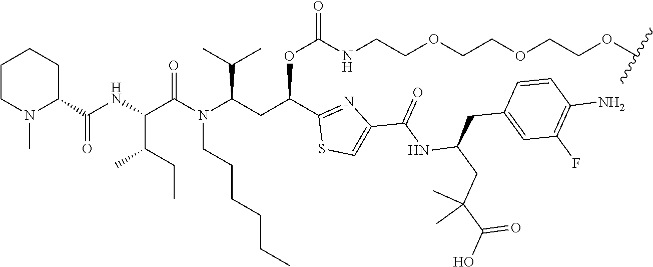 [SMILES: *OCCOCCOCCNC(=O)O[C@H](C[C@H](C(C)C)N(CCCCCC)C(=O)[C@@H](NC(=O)[C@H]1CCCCN1C)[C@@H](C)CC)c1nc(C(=O)N[C@@H](Cc2ccc(N)c(F)c2)CC(C)(C)C(=O)O)cs1] |

35. The compound of claim 27, wherein BA or the antibody or antigen-binding fragment thereof is selected from the group consisting of anti-MUC16, anti-PSMA, anti-EGFRvIII, anti-HER2, and anti-MET.

36. The compound of claim 27, wherein BA or the antibody or antigen-binding fragment thereof is anti-PRLR or anti-STEAP2.

37. The compound of claim 27, wherein BA or the antibody or antigen-binding fragment thereof binds to an antigen selected from the group consisting of lipoproteins; alpha1-antitrypsin; a cytotoxic T-lymphocyte associated antigen (CTLA), such as CTLA-4 or CTLA4; vascular endothelial growth factor (VEGF); receptors for hormones or growth factors; protein A or D; fibroblast growth factor receptor 2 (FGFR2), EpCAM or Epcam, GD3, FLT3, PSCA, MUC1 or Muc1, MUC16 or Muc16, STEAP, STEAP2 or Steap-2, CEA, TENB2, EphA receptors, EphB receptors, folate receptor, FOLRI, mesothelin, cripto, alphavbeta6, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152; erythropoietin; osteoinductive factors; immunotoxins; a bone morphogenetic protein (BMP); T-cell receptors; surface membrane proteins; integrins, such as CD11a, CD11b, CD11c, CD18, an ICAM, VLA-4, and VCAM; a tumor associated antigen such as AFP, ALK, B7H₄, BAGE proteins, β-catenin, brc-abl, BRCA1, BORIS, CA9 (carbonic anhydrase IX), caspase-8, CD123, CDK4, CLEC12A, c-kit, cMET, c-MET, MET, cyclin-B1, CYP1B1, EGFRvIII, endoglin, EphA2, ErbB2/Her2, ErbB3/Her3, ErbB4/Her4, ETV6-AML, Fra-1, FOLR1, GAGE proteins such as GAGE-1 and GAGE-2, GD2, GloboH, glypican-3, GM3, gp100, Her2 or HER2, HLA/B-raf, HLA/EBNA1, HLA/k-ras, HLA/MAGE-A3, hTERT, IGF1R, LGR5, LMP2, MAGE proteins such as MAGE-1, -2, -3, -4, -6, and -12, MART-1, ML-IAP, CA-125, MUM1, NA17, NGEP, NY-BR1, NY-BR62, NY-BR85, NY-ESO1, OX40, p15, p53, PAP, PAX3, PAX5, PCTA-1, PDGFR-α, PDGFR-β, PDGF-A, PDGF-B, PDGF-C, PDGF-D, PLAC1, PRLR, PRAME, PSGR, PSMA (FOLH1), RAGE proteins, Ras, RGS5, Rho, SART-1, SART-3, Steap-1, STn, survivin, TAG-72, TGF-β, TMPRSS2, Tn, TNFRSF17, TRP-1, TRP-2, tyrosinase, uroplakin-3, fragments of any of the above-listed polypeptides; cell-surface expressed antigens; molecules such as class A scavenger receptors including scavenger receptor A (SR-A), and other membrane proteins such as B7 family-related member including V-set and Ig domain-containing 4 (VSIG4), Colony stimulating factor 1 receptor (CSF1R), asialoglycoprotein receptor (ASGPR), and Amyloid beta precursor-like protein 2 (APLP-2); BCMA; SLAMF7; GPNMB; and UPK3A.

38. A compound having the structure of Formula (I) 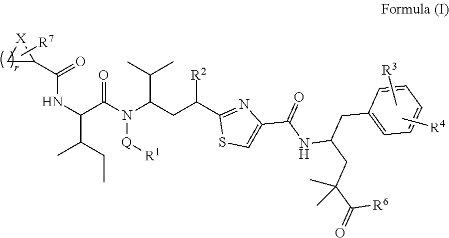
or a pharmaceutically acceptable salt thereof, wherein
X is —O— or —NR⁵,
R⁵ is a hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, or —C(O)—CH₂—NH₂;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, —O—C(O)—NH—(CH₂CH₂O)₃—CH₂NH—C(O)CH₂NH₂, or —O—C(O)—NH—(CH₂CH₂O)₂—(CH₂)₂OH;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
R⁴ is hydrogen or —F
R⁷ when present is —CH₃;
R⁶ is —OH, —NHCH₂C(O)OH, or —NH—C(O)OH;
Q is —CH₂— or —O—;
R¹ is —C₅ alkyl or —C₅ alkynyl;
r is three or four; and
wherein T is not a compound selected from the table below

| ID. | Structure |
|---|---|
| P2 | 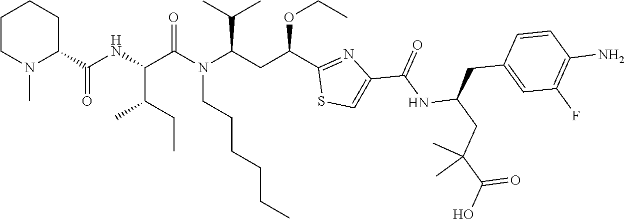 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P5 | 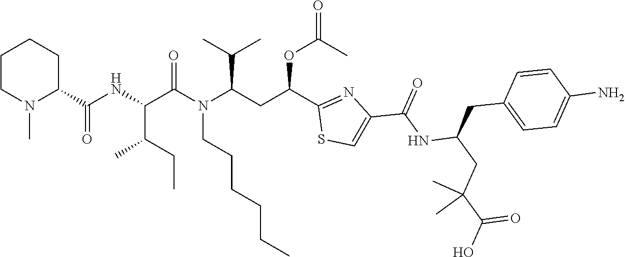 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 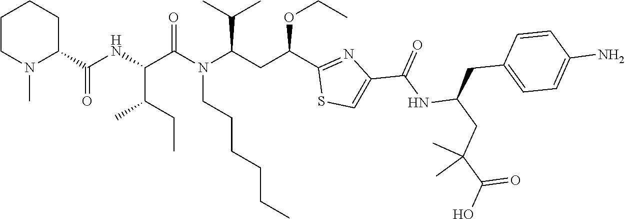 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 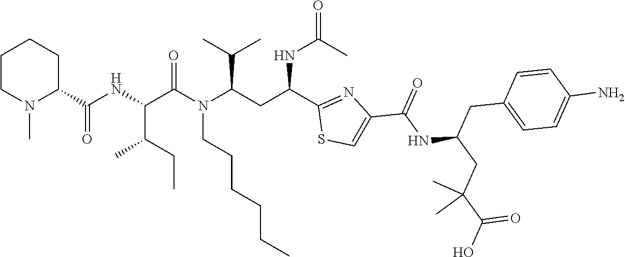 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P10 | 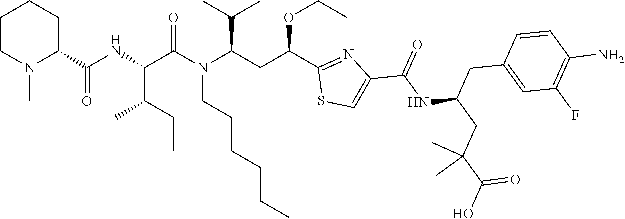 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 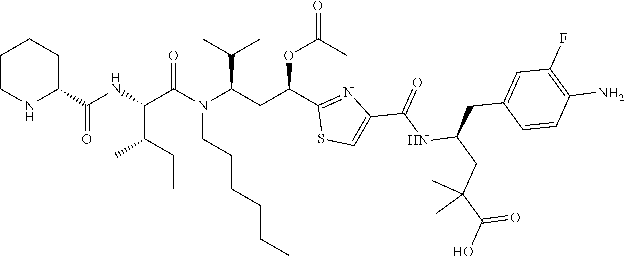 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 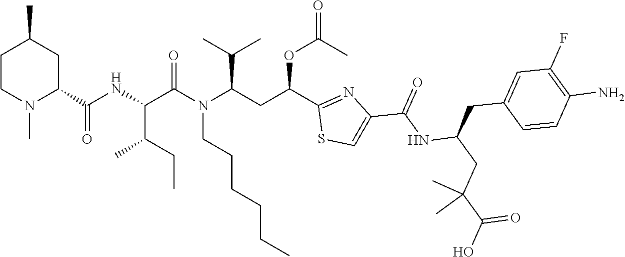 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 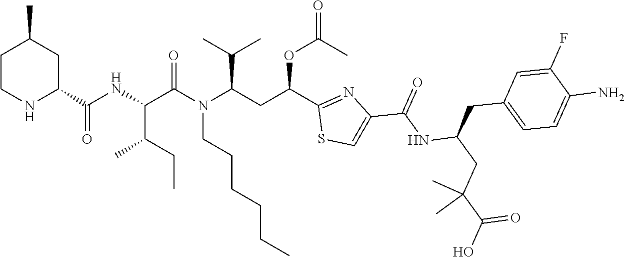 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 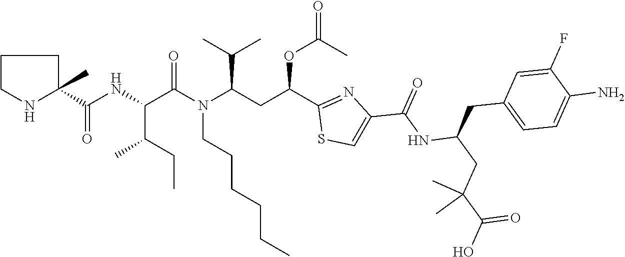 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 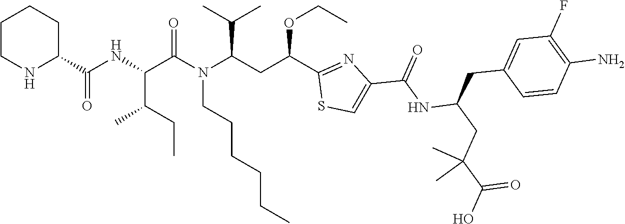 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 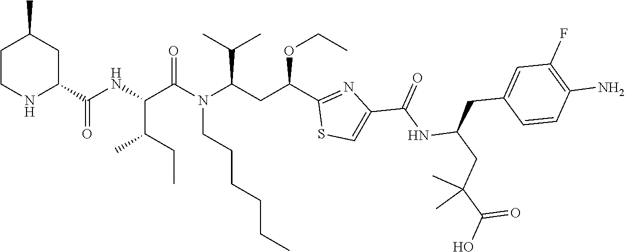 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 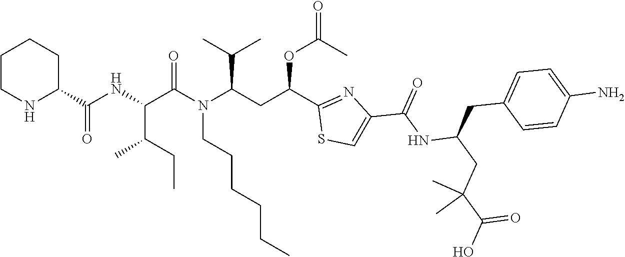 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 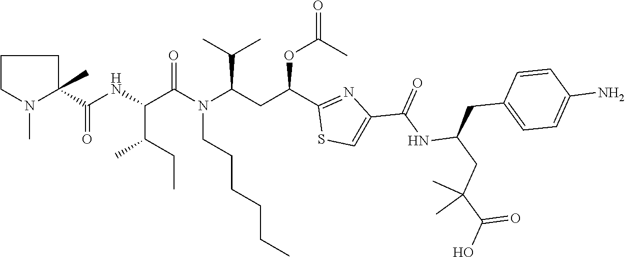 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 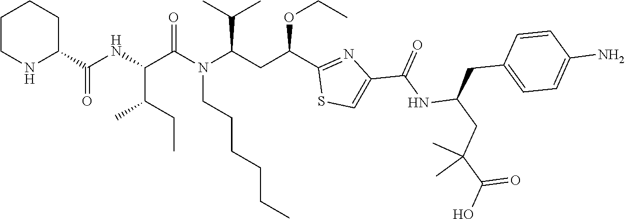 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 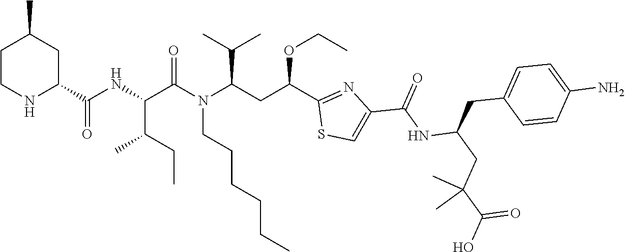 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 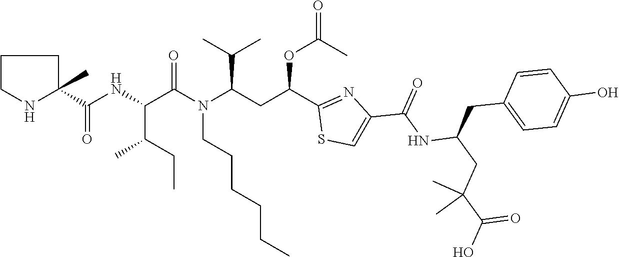 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 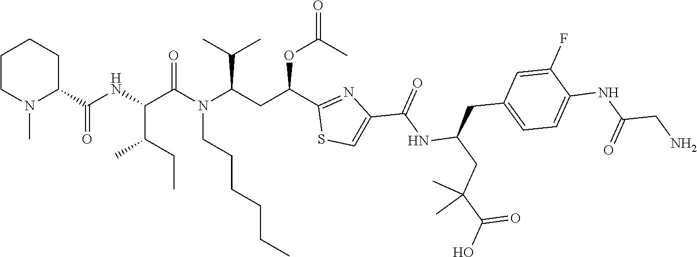 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 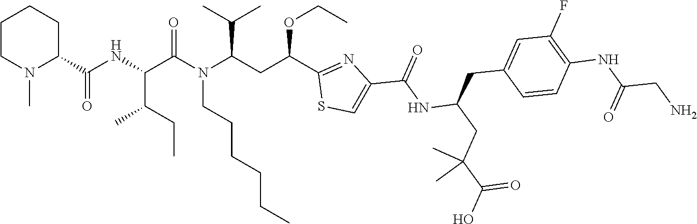 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 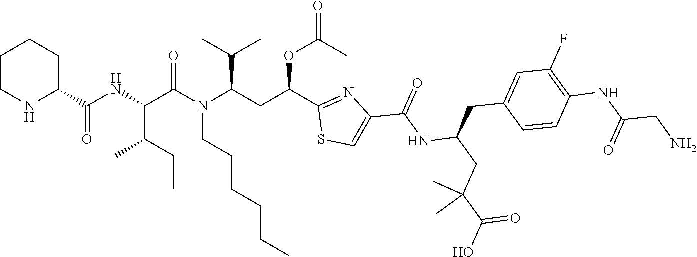 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 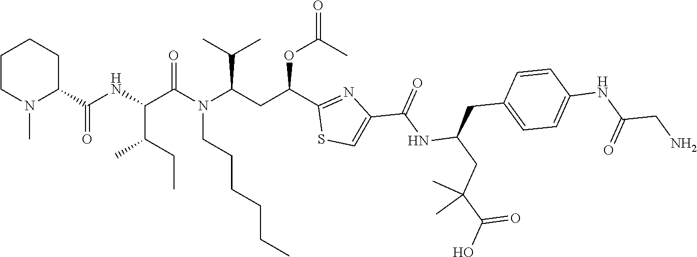 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 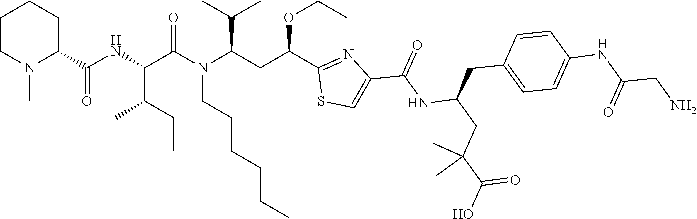 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 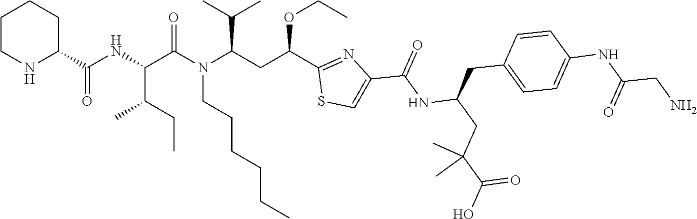 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 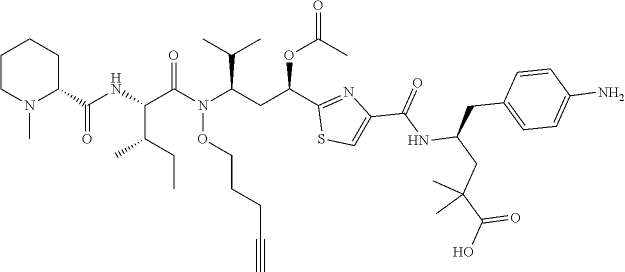 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 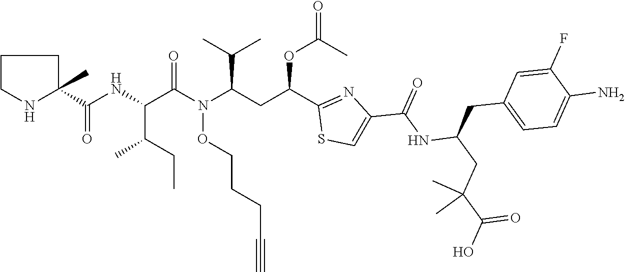 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 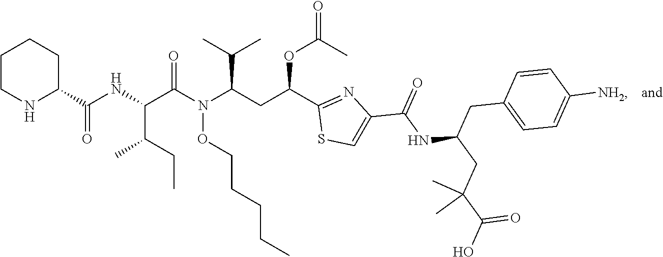 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 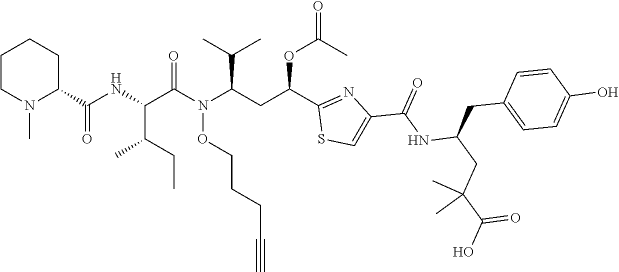 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 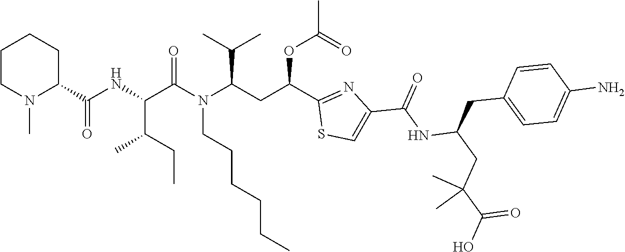 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 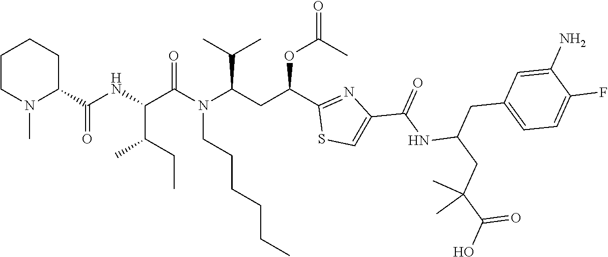 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 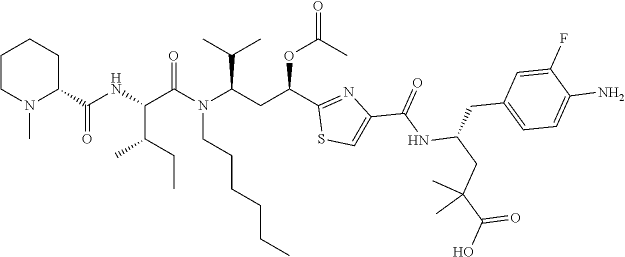 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 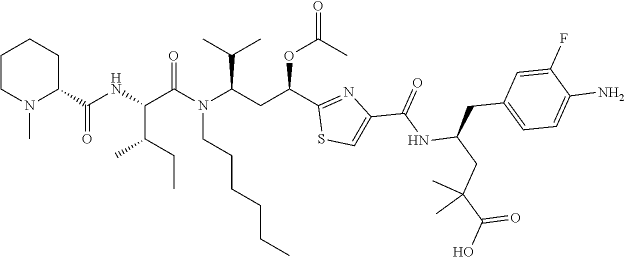 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 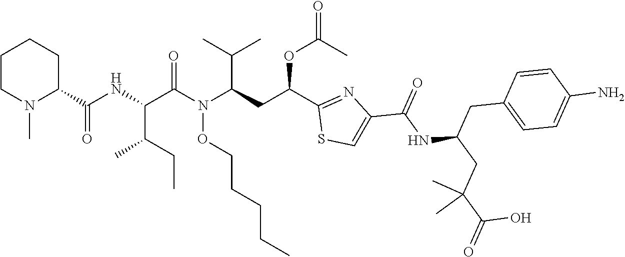 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 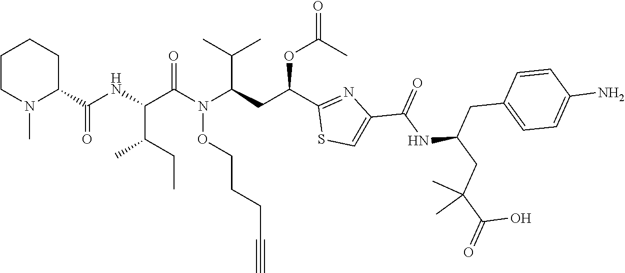 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 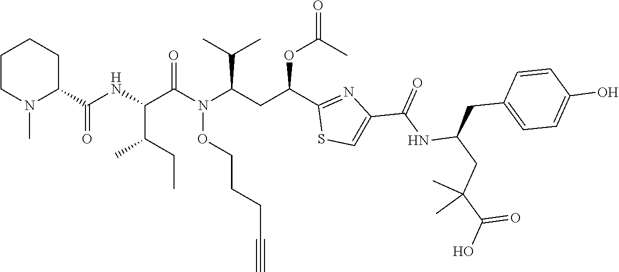 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 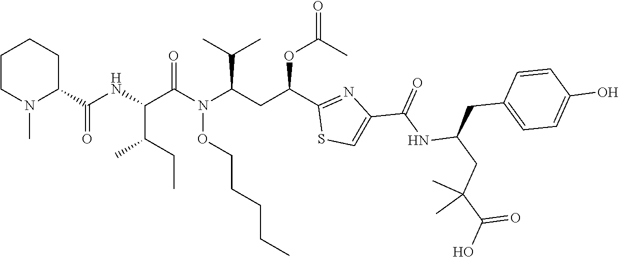 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] and |
| D-5a | 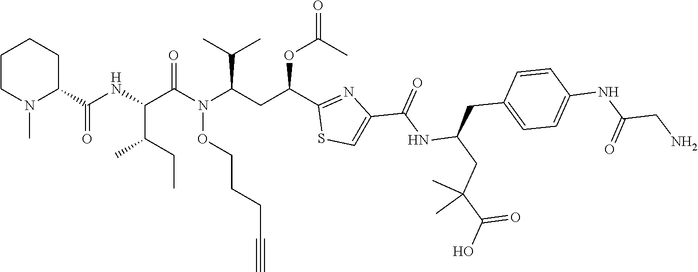 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

39. The compound of claim 38, selected from the group consisting of

| ID No./ | |
|---|---|
| Com- | |
| pound | |
| No. | Structure |
| PA1 | 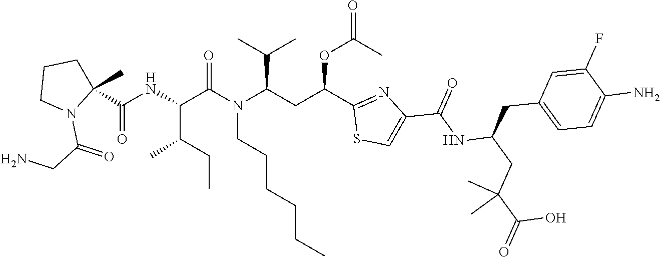 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| PA2 | 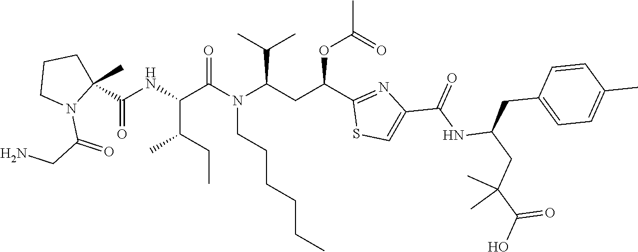 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(C)cc2)CC(C)(C)C(=O)O)cs1)C(C)C] |
| PA3 | 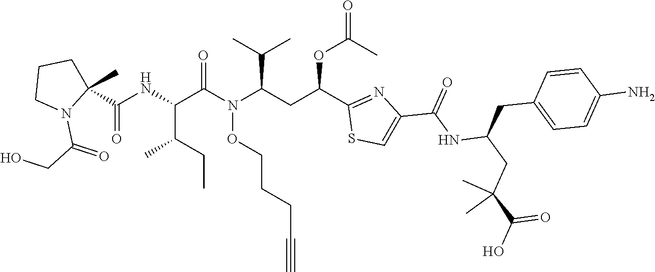 |
| PA4 | 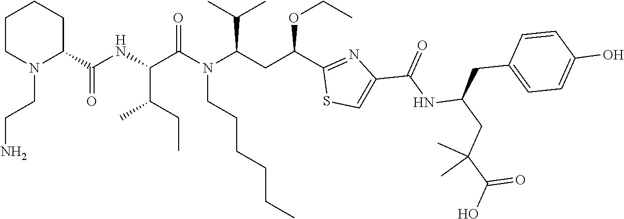 [SMILES: CCCCCCN(C(=O)[C@@H](NC(=O)[C@H]1CCCCN1CCN)[C@@H](C)CC)[C@H](C[C@@H](OCC)c1nc(C(=O)N[C@@H](Cc2ccc(O)cc2)CC(C)(C)C(=O)O)cs1)C(C)C] |
| PA5 | 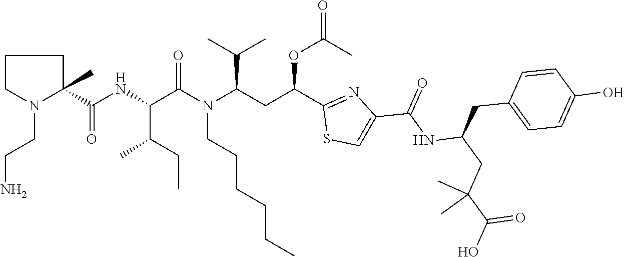 [SMILES: CCCCCCN(C(=O)[C@@H](NC(=O)[C@@]1(C)CCCN1CCN)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| PA6 | 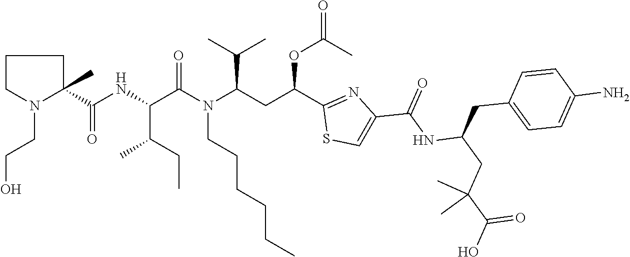 [SMILES: CCCCCCN(C(=O)[C@@H](NC(=O)[C@@]1(C)CCCN1CCO)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| PA7 | 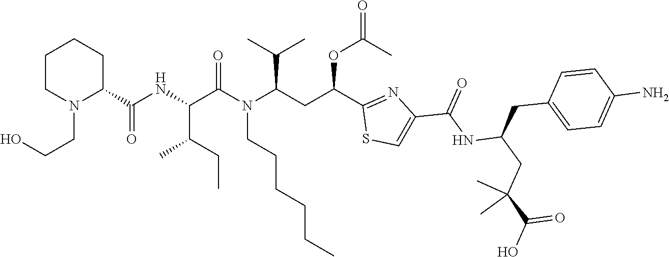 |
| PA8 | 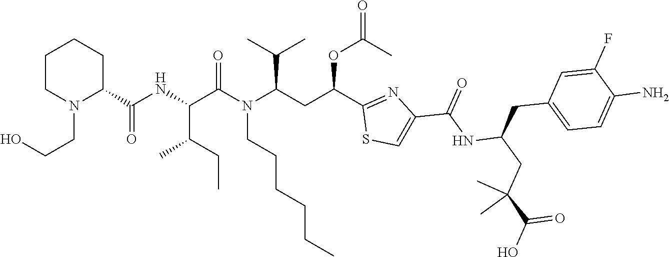 |
| PA9 | 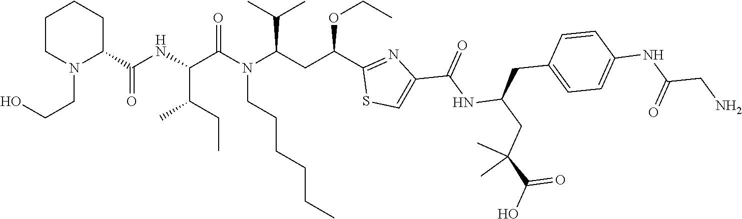 |
| PA10 | 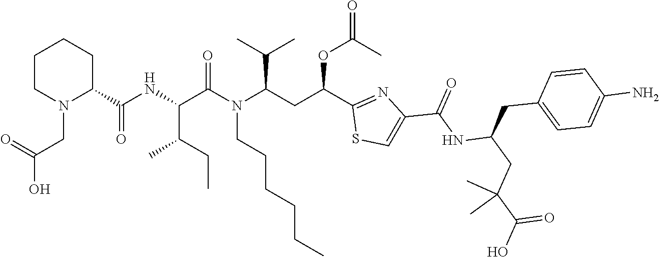 [SMILES: CCCCCCN(C(=O)[C@@H](NC(=O)[C@H]1CCCCN1CC(=O)O)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| PA11 | 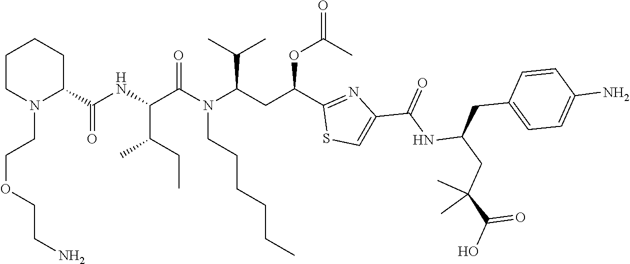 |
| PA12 | 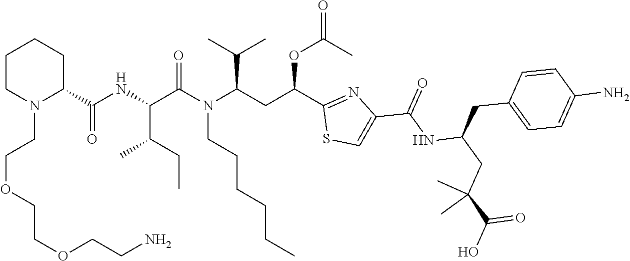 |
| PA13 | 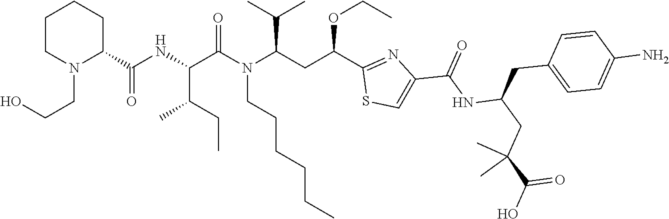 |
| PA14 | 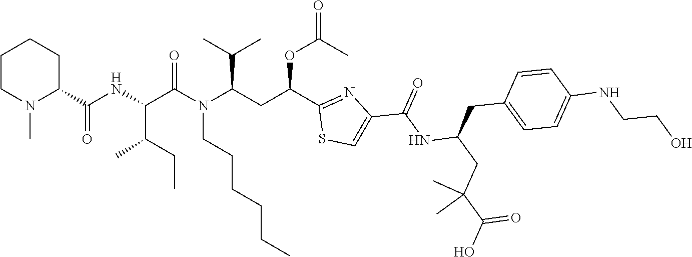 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCCO)cc2)CC(C)(C)C(=O)O)cs1)C(C)C] |
| PA15 | 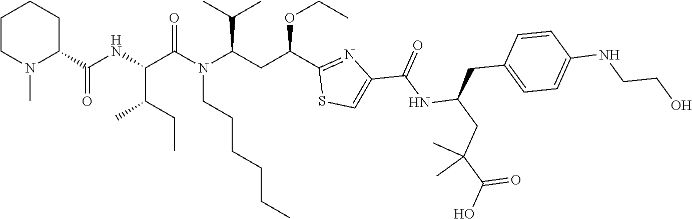 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCO)cc2)CC(C)(C)C(=O)O)cs1)C(C)C] |
| PA16 | 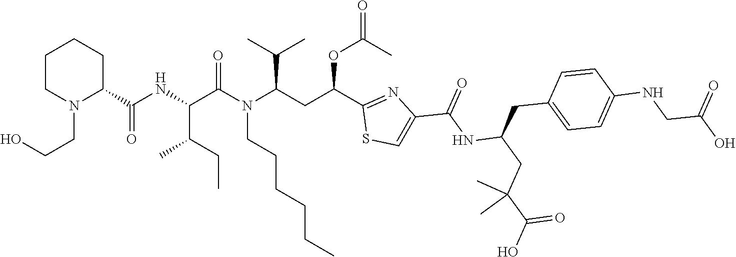 [SMILES: CCCCCCN(C(=O)[C@@H](NC(=O)[C@H]1CCCCN1CCO)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C] |
| PA17 | 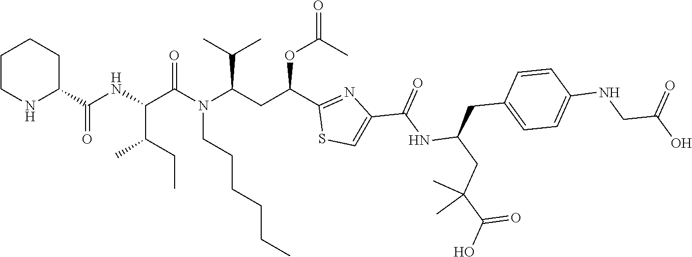 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C] |
| PA18 | 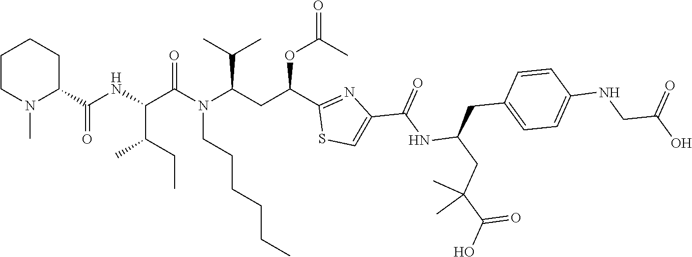 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C] |
| PA19 | 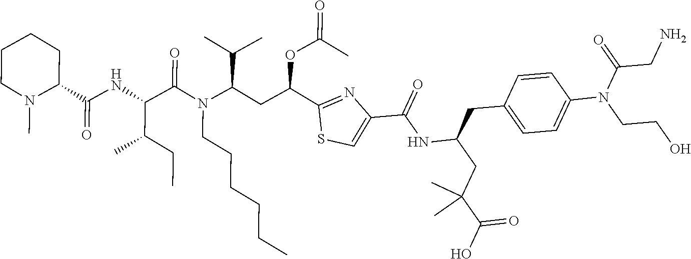 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N(CCO)C(=O)CN)cc2)CC(C)(C)C(=O)O)cs1)C(C)C] |
| PA20 | 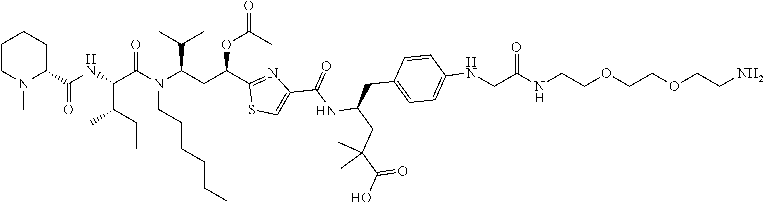 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NCC(=O)NCCOCCOCCN)cc2)CC(C)(C)C(=O)O)cs1)C(C)C] |
| PA21 | 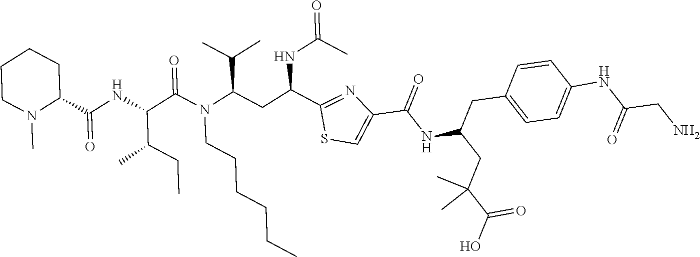 |
| PA22 | 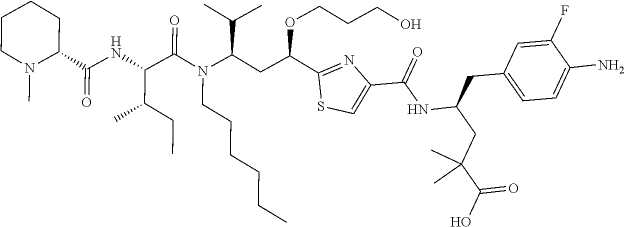 [SMILES: CCCCCCN(C(=O)[C@@H](NC(=O)[C@H]1CCCCN1C)[C@@H](C)CC)[C@H](C[C@@H](OCCCO)c1nc(C(=O)N[C@@H](Cc2ccc(N)c(F)c2)CC(C)(C)C(=O)O)cs1)C(C)C] |
| PA23 | 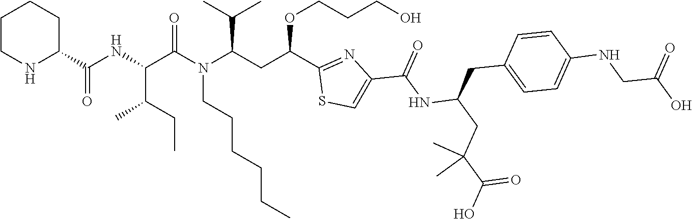 [SMILES: CCCCCCN(C(=O)[C@@H](NC(=O)[C@H]1CCCCN1)[C@@H](C)CC)[C@H](C[C@@H](OCCCO)c1nc(C(=O)N[C@@H](Cc2ccc(NCC(=O)O)cc2)CC(C)(C)C(=O)O)cs1)C(C)C] |
| PA24 | 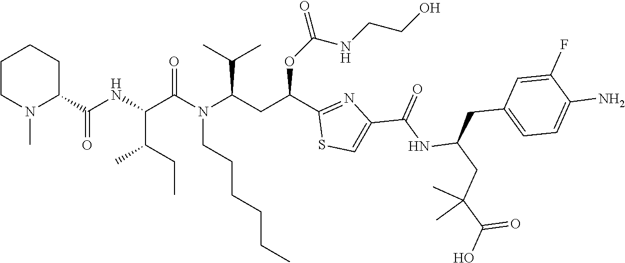 [SMILES: CCCCCCN(C(=O)[C@@H](NC(=O)[C@H]1CCCCN1C)[C@@H](C)CC)[C@H](C[C@@H](OC(=O)NCCO)c1nc(C(=O)N[C@@H](Cc2ccc(N)c(F)c2)CC(C)(C)C(=O)O)cs1)C(C)C] |
| PA25 | 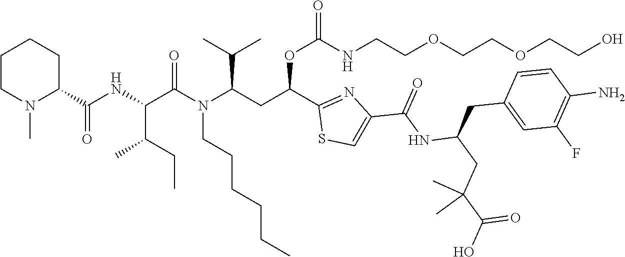 [SMILES: CCCCCCN(C(=O)[C@@H](NC(=O)[C@H]1CCCCN1C)[C@@H](C)CC)[C@H](C[C@@H](OC(=O)NCCOCCOCCO)c1nc(C(=O)N[C@@H](Cc2ccc(N)c(F)c2)CC(C)(C)C(=O)O)cs1)C(C)C] |
| PA26 | 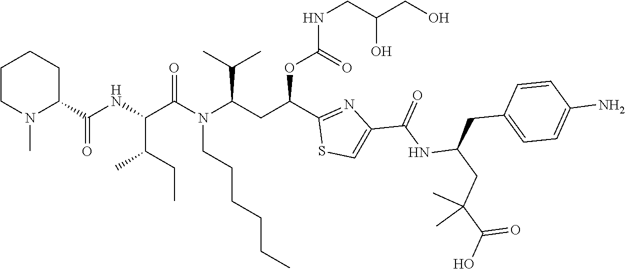 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(N)cc2)CC(C)(C)C(=O)O)cs1)C(C)C] |
| PA27 | 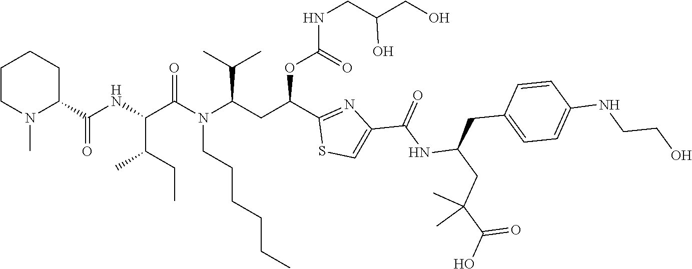 [SMILES: CCCCCCN(C(=O)[C@@H](NC(=O)[C@H]1CCCCN1C)[C@@H](C)CC)[C@H](C[C@@H](OC(=O)NCC(O)CO)c1nc(C(=O)N[C@@H](Cc2ccc(NCCO)cc2)CC(C)(C)C(=O)O)cs1)C(C)C] |
| PA28 | 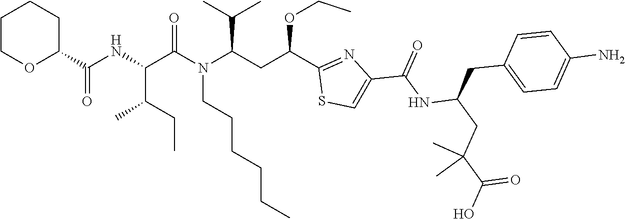 [SMILES: CCCCCCN(C(=O)[C@@H](NC(=O)[C@H]1CCCCO1)[C@@H](C)CC)[C@H](C[C@@H](OCC)c1nc(C(=O)N[C@@H](Cc2ccc(N)cc2)CC(C)(C)C(=O)O)cs1)C(C)C] |
| PA29 | 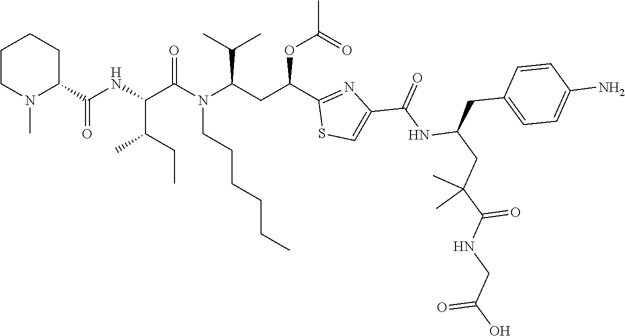 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)NCC(=O)O)cs1)C(C)C] |
| PA30 | 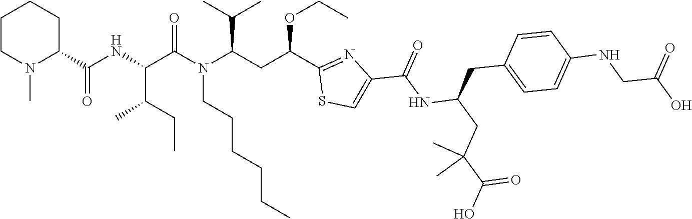 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCC(=O)O)cc2)CC(C)(C)C(=O)O)cs1)C(C)C] |

40. The compound of claim 38, wherein
Q is —CH₂— or —O—;
X is —O— or —NR⁵,
R⁵ is hydrogen, —CH₃, —(CH₂)₂—OH, —(CH₂)₂—NH₂, —CH₂—C(O)—OH, —(CH₂)₂—O—(CH₂)₂—NH₂, or —C(O)—CH₂—NH₂;
R¹ is —C₅ alkyl or —C₅ alkynyl;
R⁶ is —OH or —NH—C(O)OH;
R⁷ when present is —CH₃; and
r is three or four.

41. The compound of claim 40, according to the structure of Formula (II) 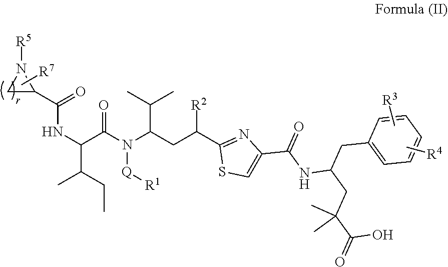
or a pharmaceutically acceptable salt thereof.

42. The compound of claim 41, wherein R⁵ is —(CH₂)₂—OH or —(CH₂)₂—NH₂.

43. The compound of claim 42, selected from the group consisting of 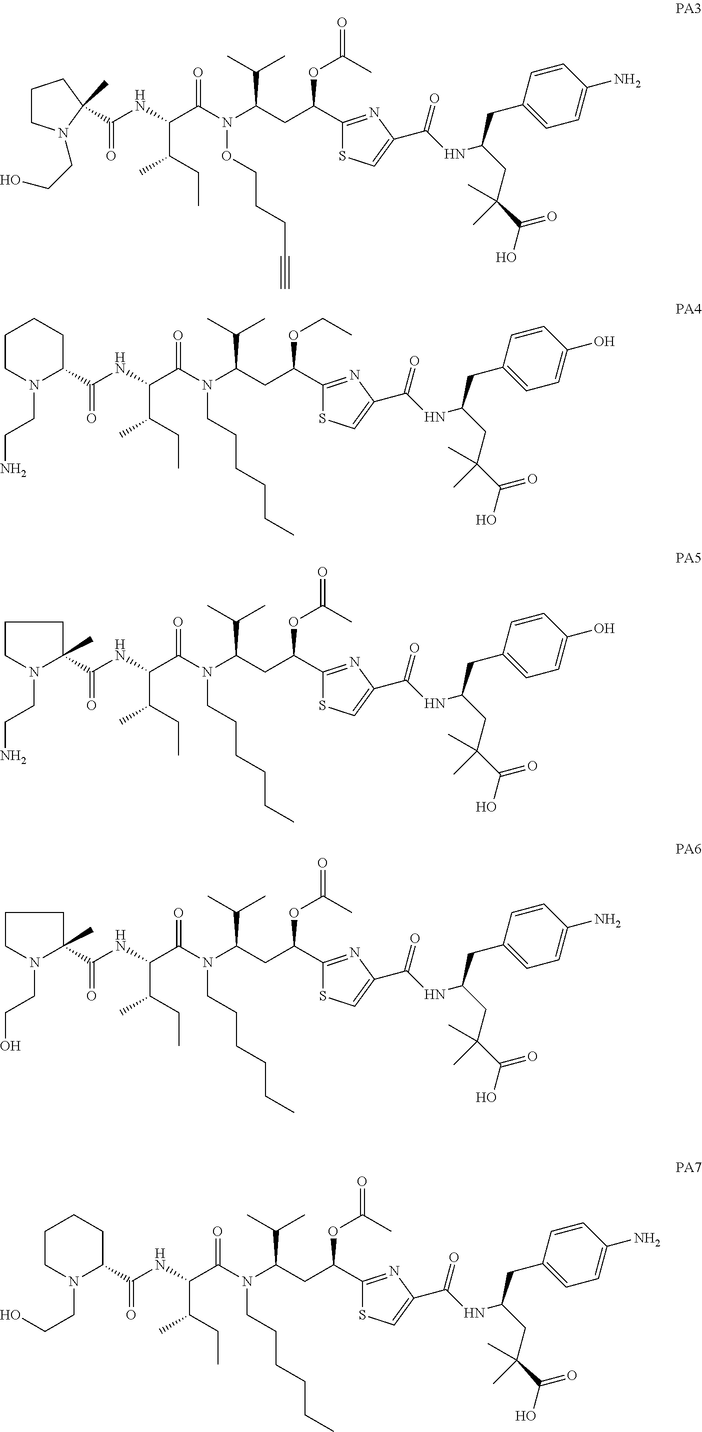 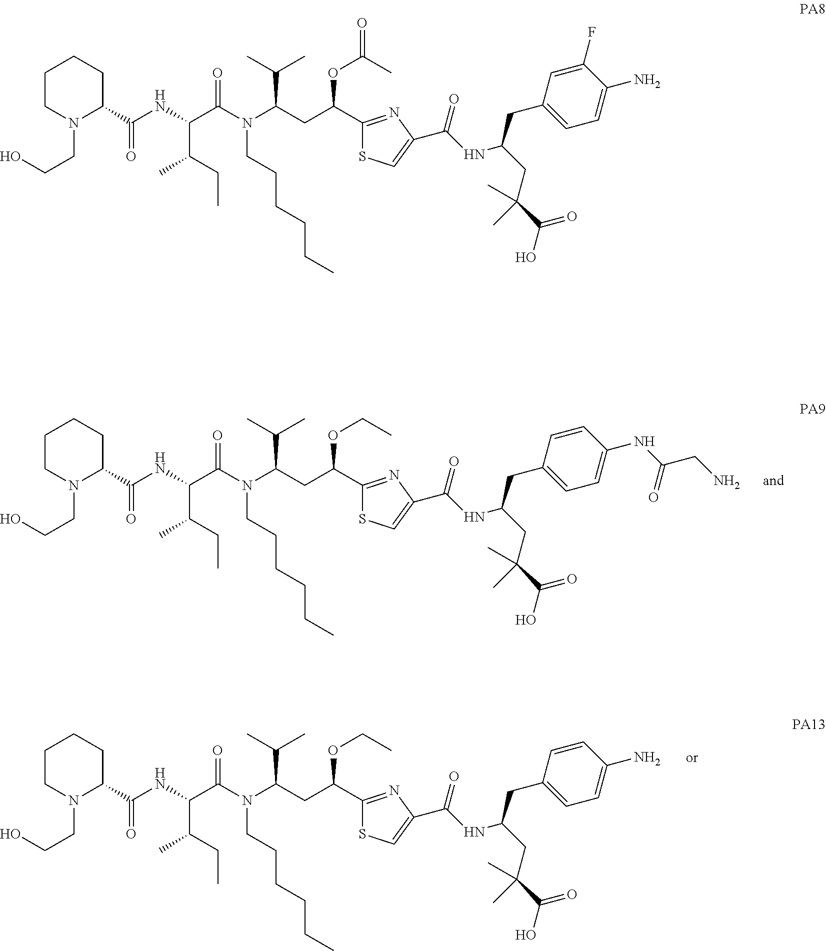
a pharmaceutically acceptable salt thereof.

44. The compound of claim 41, wherein R⁵ is —CH₂—C(O)—OH or —C(O)—CH₂—NH₂.

45. The compound of claim 43, selected from the group consisting of 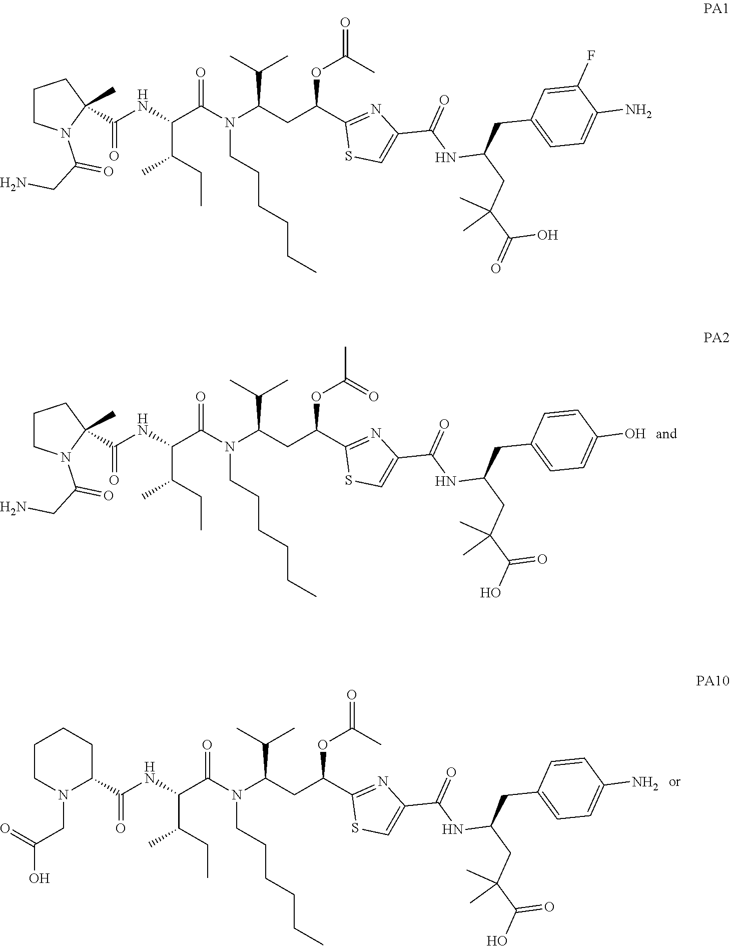 [SMILES: CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C ; CCCCCCN(C(=O)[C@@H](NC(=O)[C@@]1(C)CCCN1C(=O)CN)[C@@H](C)CC)[C@H](C[C@@H](OC(C)=O)c1nc(C(=O)N[C@@H](Cc2ccc(O)cc2)CC(C)(C)C(=O)O)cs1)C(C)C ; CCCCCCN(C(=O)[C@@H](NC(=O)[C@H]1CCCCN1CC(=O)O)[C@@H](C)CC)[C@H](C[C@@H](OC(C)=O)c1nc(C(=O)N[C@@H](Cc2ccc(N)cc2)CC(C)(C)C(=O)O)cs1)C(C)C]
a pharmaceutically acceptable salt thereof.

46. The compound of claim 40, according to the structure of Formula (III) 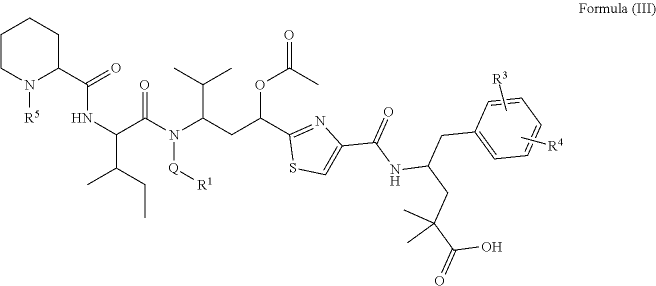
or a pharmaceutically acceptable salt thereof.

47. The compound of claim 46, wherein R⁵ is —(CH₂)₂—O—(CH₂)₂—NH₂ or —(CH₂CH₂—O)₂—(CH₂)₂—NH₂.

48. The compound of claim 47, selected from the group consisting of 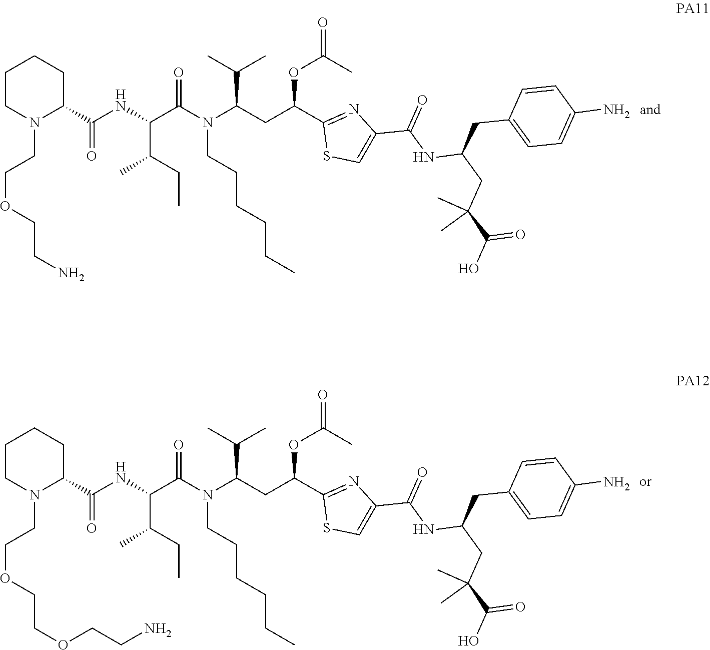
a pharmaceutically acceptable salt thereof.

49. The compound of claim 40, according to the structure of Formula (IV) 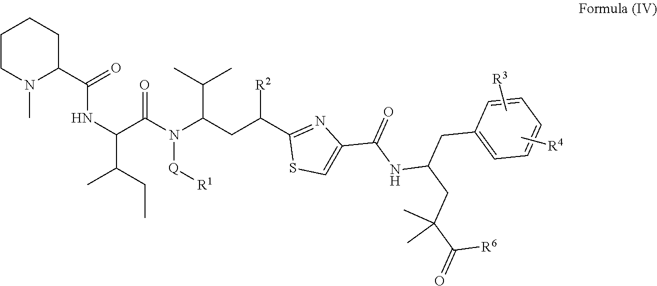
or a pharmaceutically acceptable salt thereof.

50. The compound of claim 49, wherein R² is —O—C(O)CH₃, —O—C(O)—NH—(CH₂)₂—OH, —O—C(O)—NH—CH₂—CH(OH)—CH₂OH, —O—C(O)—NH—(CH₂CH₂O)₂—(CH₂)₂OH, —NH—CH₂—C(O)—NH—(CH₂CH₂O)₂—(CH₂)₂NH₂, and R⁶ is —OH.

51. The compound of claim 50, selected from the group consisting of 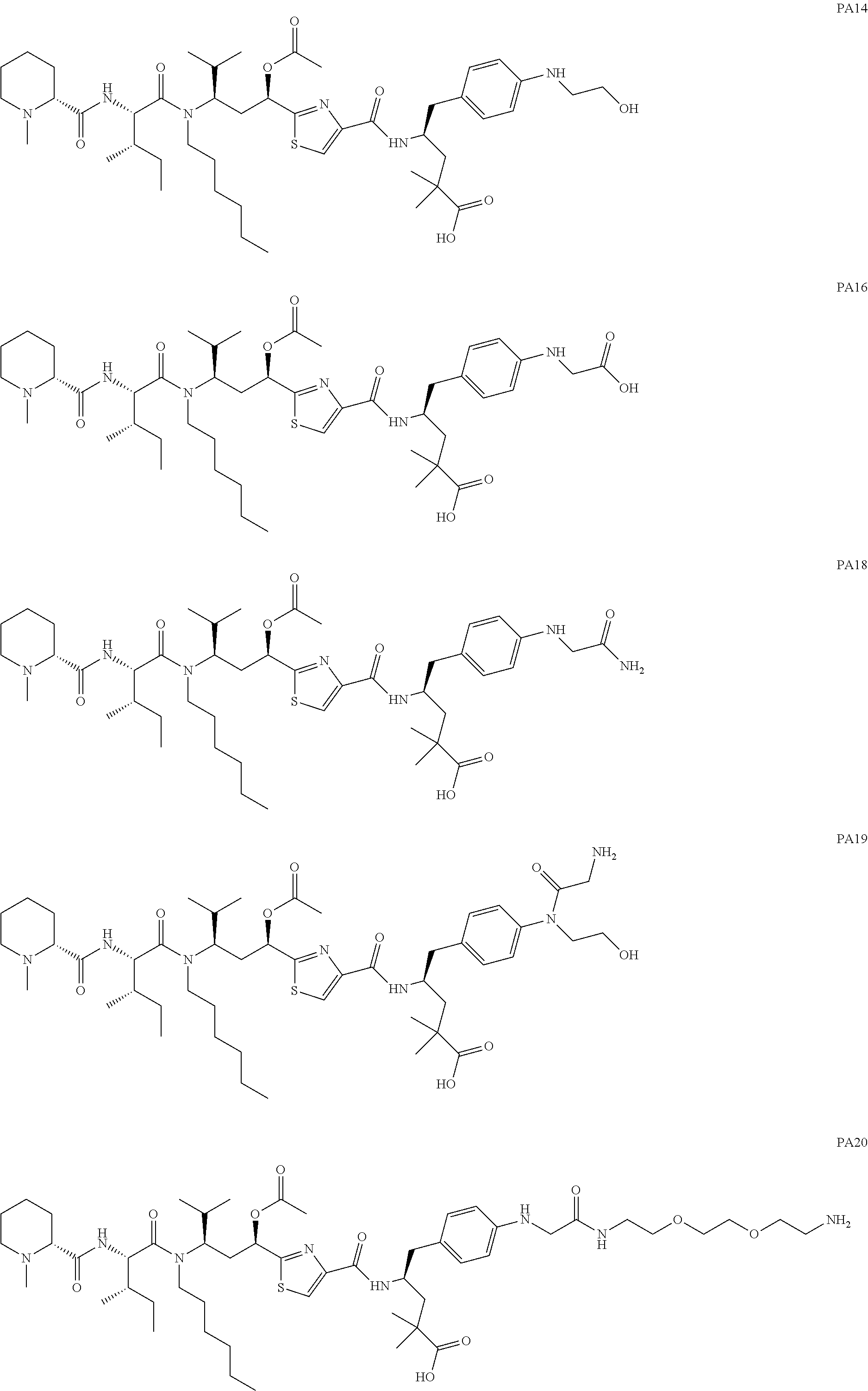 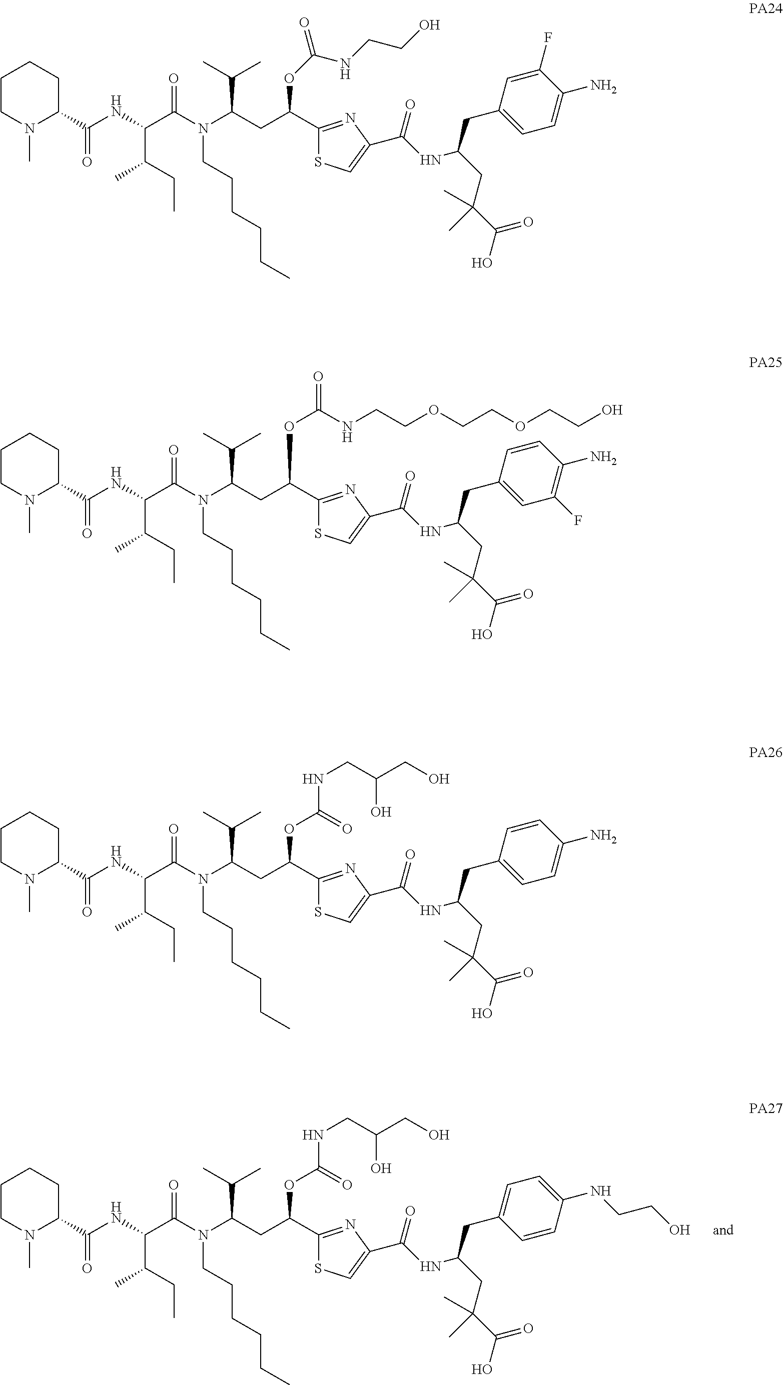 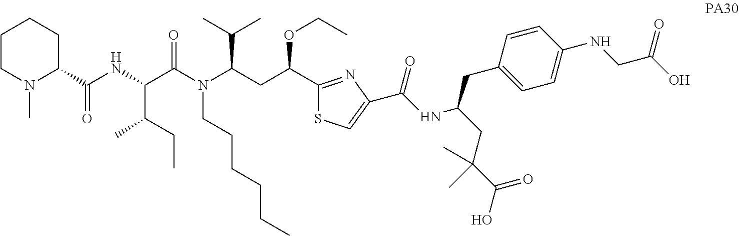 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCC(=O)O)cc2)CC(C)(C)C(=O)O)cs1)C(C)C]
or
a pharmaceutically acceptable salt thereof.

52. The compound of claim 49, wherein R² is —O—C(O)CH₃ and R⁶ is —NH—C(O)OH or —NHCH₂C(O)OH.

53. The compound of claim 52, having the following structure: 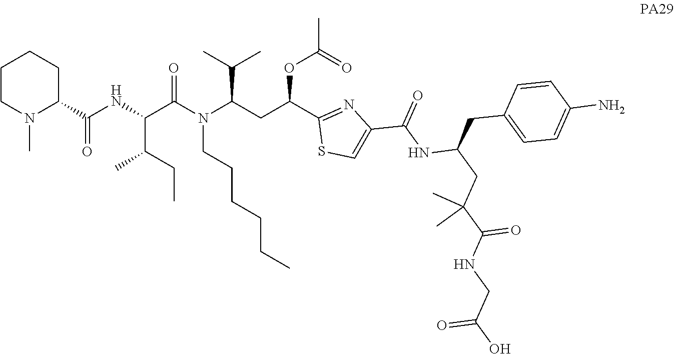 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)NCC(=O)O)cs1)C(C)C]
or
a pharmaceutically acceptable salt thereof.

54. The compound of claim 49, wherein R² is —O—CH₂CH₃ or —O—(CH₂)₃—OH, and R⁶ is —OH.

55. The compound of claim 54, selected from the group consisting of 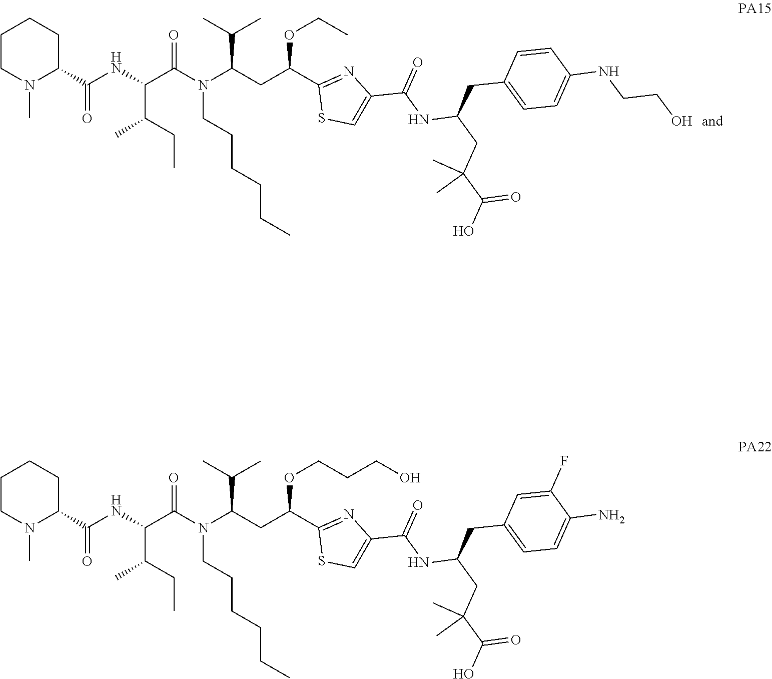 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NCCO)cc2)CC(C)(C)C(=O)O)cs1)C(C)C ; CCCCCCN(C(=O)[C@@H](NC(=O)[C@H]1CCCCN1C)[C@@H](C)CC)[C@H](C[C@@H](OCCCO)c1nc(C(=O)N[C@@H](Cc2ccc(N)c(F)c2)CC(C)(C)C(=O)O)cs1)C(C)C]
or
a pharmaceutically acceptable salt thereof.

56. The compound of claim 49, wherein R² is —N—C(O)CH₃ and R⁶ is —OH.

57. The compound of claim 56, having the following structure 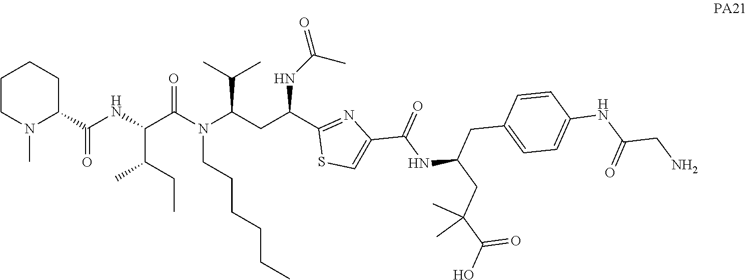 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(NC(=O)CN)cc2)CC(C)(C)C(=O)O)cs1)C(C)C]
or
a pharmaceutically acceptable salt thereof.

58. The compound of claim 40, according to the structure of Formula (V) 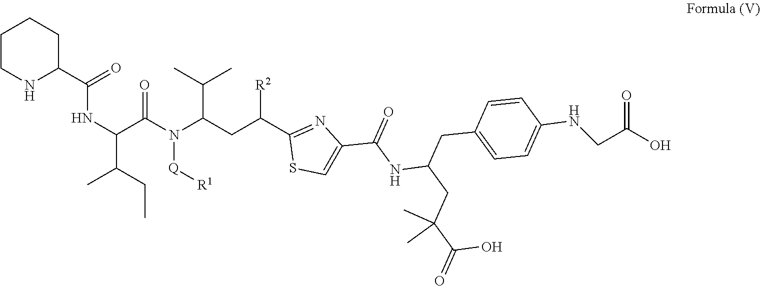
or a pharmaceutically acceptable salt thereof.

59. The compound of claim 58, wherein R² is —O—C(O)CH₃ or —O—(CH₂)₃—OH.

60. The compound of claim 59, selected from the group consisting of 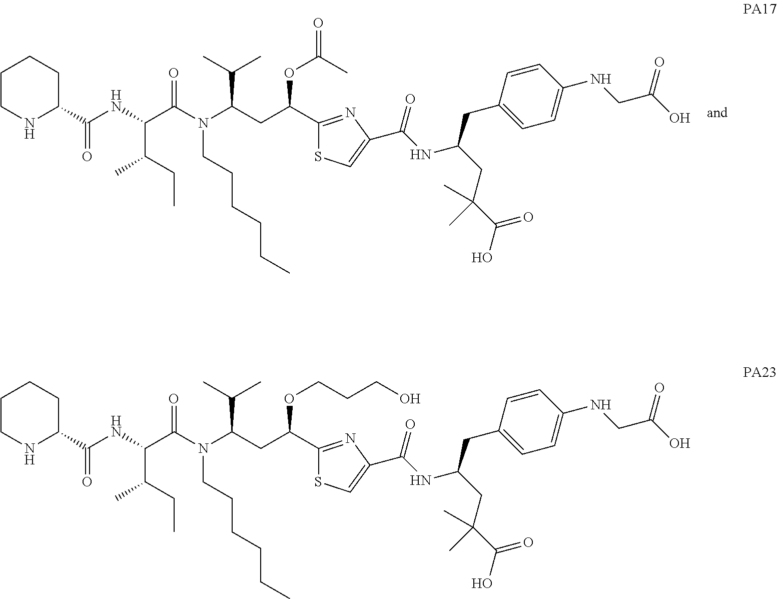 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NCC(=O)O)cc2)CC(C)(C)C(=O)O)cs1)C(C)C ; CCCCCCN(C(=O)[C@@H](NC(=O)[C@H]1CCCCN1)[C@@H](C)CC)[C@H](C[C@@H](OCCCO)c1nc(C(=O)N[C@@H](Cc2ccc(NCC(=O)O)cc2)CC(C)(C)C(=O)O)cs1)C(C)C]
or
a pharmaceutically acceptable salt thereof.

61. The compound of claim 40, according to the structure of Formula (VI) 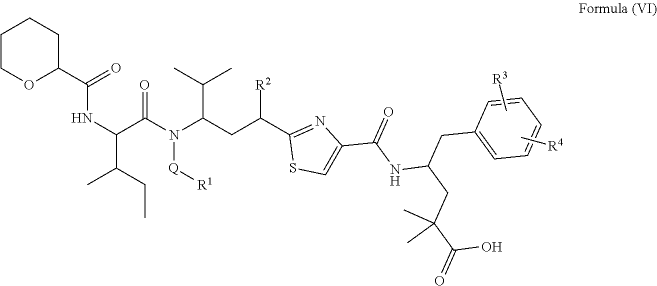
or a pharmaceutically acceptable salt thereof.

62. The compound of claim 61, wherein R² is —O—C(O)CH₃.

63. The compound of claim 62, having the following structure 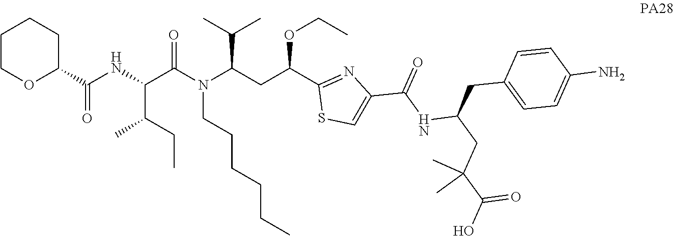 [SMILES: CCCCCCN(C(=O)[C@@H](NC(=O)[C@H]1CCCCO1)[C@@H](C)CC)[C@H](C[C@@H](OCC)c1nc(C(=O)N[C@@H](Cc2ccc(N)cc2)CC(C)(C)C(=O)O)cs1)C(C)C]
or
a pharmaceutically acceptable salt thereof.

64. A pharmaceutical composition comprising the compound of claim 1 and a pharmaceutically acceptable excipient, carrier, or diluent.

65. A pharmaceutical composition comprising the compound of claim 38 and a pharmaceutically acceptable excipient, carrier, or diluent.

66. A method for treating cancer in a subject comprising administering to the subject an effective treatment amount of a pharmaceutical composition of claim 64.

67. A method for treating cancer in a subject comprising administering to the subject an effective treatment amount of a pharmaceutical composition of claim 65.

68. A method for treating cancer in a subject comprising administering to the subject an effective treatment amount of a pharmaceutical composition of claim 64, wherein the cancer is selected from the group consisting of renal cell carcinoma, pancreatic carcinoma, head and neck cancer, prostate cancer, castrate-resistant prostrate cancer, malignant gliomas, osteosarcoma, colorectal cancer, gastric cancer, mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer, and Wilms' tumor.

69. A method for treating cancer in a subject comprising administering to the subject an effective treatment amount of a pharmaceutical composition of claim 65, wherein the cancer is selected from the group consisting of renal cell carcinoma, pancreatic carcinoma, head and neck cancer, prostate cancer, castrate-resistant prostrate cancer, malignant gliomas, osteosarcoma, colorectal cancer, gastric cancer, mesothelioma, malignant mesothelioma, multiple myeloma, ovarian cancer, lung cancer, small cell lung cancer, non-small cell lung cancer, synovial sarcoma, thyroid cancer, breast cancer, PRLR positive (PRLR+) breast cancer, melanoma, acute myelogenous leukemia, adult T-cell leukemia, astrocytomas, bladder cancer, cervical cancer, cholangiocarcinoma, endometrial cancer, esophageal cancer, glioblastomata, Kaposi's sarcoma, kidney cancer, leiomyosarcomas, liver cancer, lymphomas, MFH/fibrosarcoma, nasopharyngeal cancer, rhabdomyosarcoma, colon cancer, stomach cancer, uterine cancer, residual cancer, and Wilms' tumor.

70. A method for treating tumors that express an antigen selected from the group consisting of PRLR and STEAP2 comprising administering to the subject an effective treatment amount of a pharmaceutical composition of claim 1.

71. A linker-payload having the formula
L-T
or a pharmaceutically acceptable salt thereof, wherein
L is a linker covalently bound to T;
T is 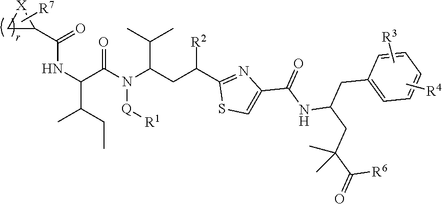
covalently bound to L, wherein
X is —O— or —NR⁵;
R⁵ is hydrogen, —CH₃, —(CH₂)₂—OH, —(CH₂)₂—NH₂, —CH₂—C(O)—OH, —(CH₂)₂—O—(CH₂)₂—NH₂, —(CH₂CH₂—O)₂—(CH₂)₂—NH₂, —C(O)—CH₂—NH₂, a first N-terminal amino acid residue, a first amino acid residue; or
R⁵ is a covalent bond to L; or
R⁵ is —(CH₂)₂—OH or —CH₂—C(O)—OH with a covalent bond to L from the terminal oxygen in —(CH₂)₂—OH or —CH₂—C(O)—OH; or
R⁵ is —(CH₂)₂—NH₂, —(CH₂)₂—O—(CH₂)₂—NH₂, —(CH₂CH₂—O)₂—(CH₂)₂—NH₂, —C(O)—CH₂—NH₂, a first N-terminal amino acid residue, or a first amino acid residue with a covalent bond to L from the nitrogen in any one of —(CH₂)₂—NH₂,
—(CH₂)₂—O—(CH₂)₂—NH₂, —(CH₂CH₂—O)₂—(CH₂)₂—NH₂, or —C(O)—CH₂—NH₂, the first N-terminal amino acid residue, or the first amino acid residue;
R² is —O—C(O)—NH—CH₂—CH(OH)—CH₂OH, —NH—C(O)CH₃, —O—CH₂CH₃, —O—(CH₂)₃—OH, —O—C(O)CH₃, —O—C(O)—NH—(CH₂)₂—OH, —O—C(O)—NH—(CH₂CH₂O)₃—CH₂NH—C(O)CH₂NH₂, or —O—C(O)—NH—(CH₂CH₂O)₂—(CH₂)₂OH; or
R² is —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH with a covalent bond to L from a terminal oxygen in any one of —O—C(O)—NH—CH₂—CH(OH)—CH₂OH, —O—(CH₂)₃—OH, —O—C(O)—NH—(CH₂)₂—OH, or —O—C(O)—NH—(CH₂CH₂O)₂—(CH₂)₂OH; or
R² is —O—C(O)—NH—(CH₂CH₂O)₃—CH₂NH—C(O)CH₂NH₂ with a covalent bond to L from the terminal nitrogen in —O—C(O)—NH—(CH₂CH₂O)₃—CH₂NH—C(O)CH₂NH₂;
R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂; or
R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or
R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂;
R⁴ is hydrogen or —F;
R⁷ when present is —CH₃;
R⁶ is —OH, —NHCH₂C(O)OH, or —NH—C(O)OH; or
R⁶ is —OH, —NHCH₂C(O)OH, or —NH—C(O)OH with a covalent bond to L from the terminal oxygen in any one of —OH, —NHCH₂C(O)OH, or —NH—C(O)OH;
Q is —CH₂— or —O—;
R¹ is —C₅ alkyl, —C₆ alkyl or —C₅ alkynyl;
r is three or four; and
k is an integer from one to thirty;
wherein T is not a compound selected from the table below

| ID. | Structure |
|---|---|
| P2 | 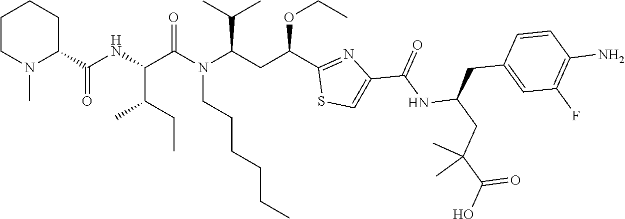 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P5 | 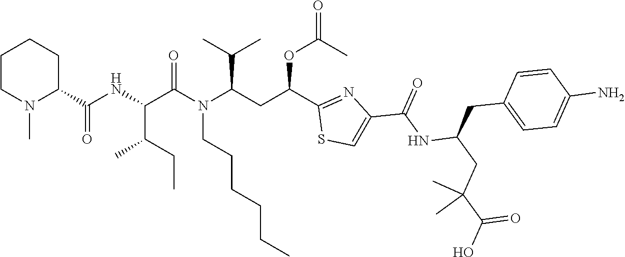 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P6 | 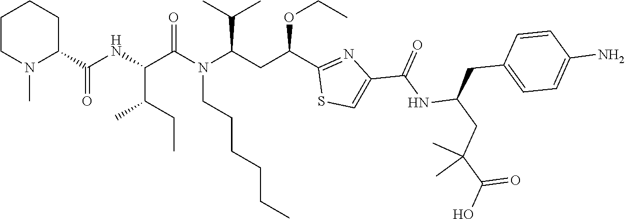 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)cc2)CC(C)(C)C(=O)O)cs1)C(C)C] |
| P7 | 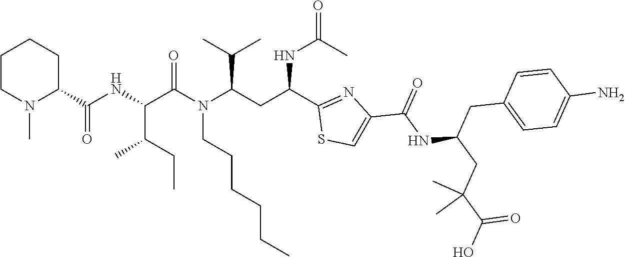 [SMILES: CCCCCCN(C(=O)[C@@H](NC(=O)[C@H]1CCCCN1C)[C@@H](C)CC)[C@H](C[C@@H](NC(C)=O)c1nc(C(=O)N[C@@H](Cc2ccc(N)cc2)CC(C)(C)C(=O)O)cs1)C(C)C] |
| P10 | 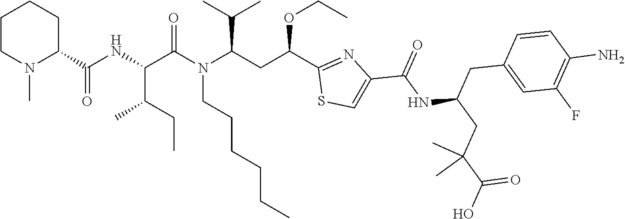 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P12 | 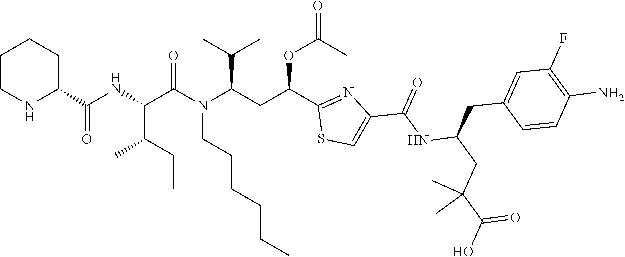 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P13 | 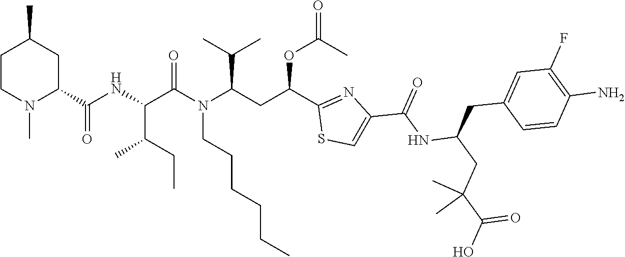 [SMILES: CCCCCCN(C(=O)[C@@H](NC(=O)[C@H]1C[C@H](C)CCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P14 | 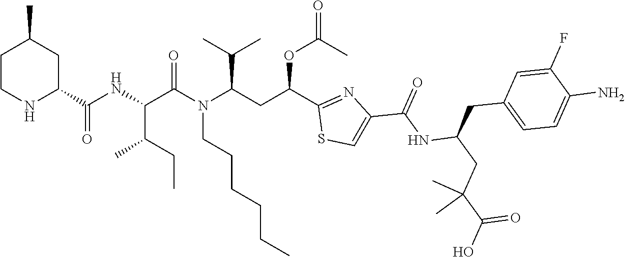 [SMILES: CCCCCCN(C(=O)[C@@H](NC(=O)[C@H]1C[C@H](C)CCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P15 | 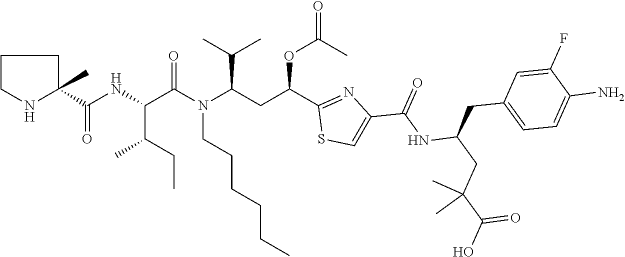 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P16 | 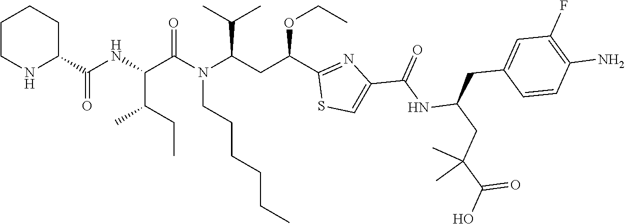 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P17 | 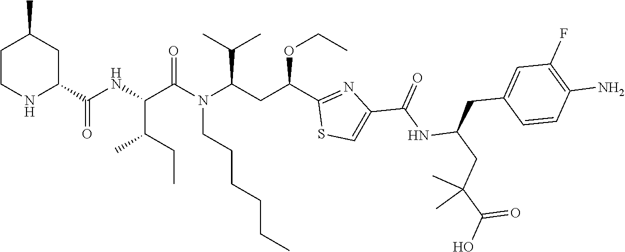 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)c(F)c2)CC(C)(C)C(=O)O)cs1)C(C)C] |
| P18 | 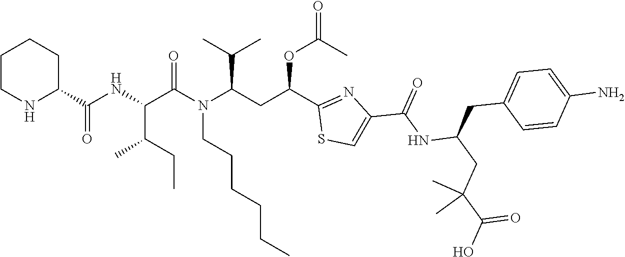 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P19 | 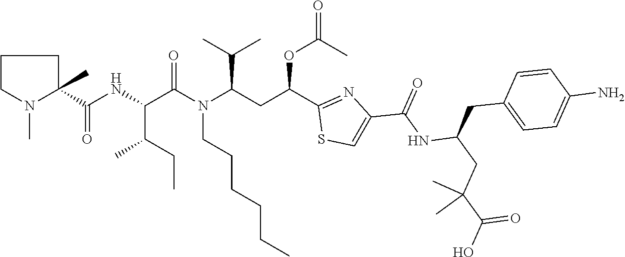 [SMILES: CCCCCCN(C(=O)[C@@H](NC(=O)[C@@]1(C)CCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P20 | 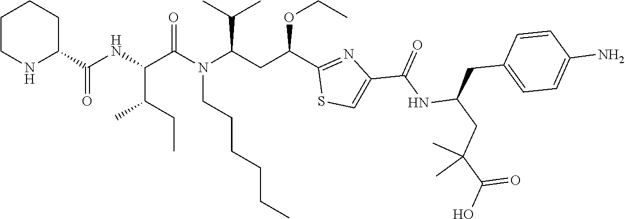 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P21 | 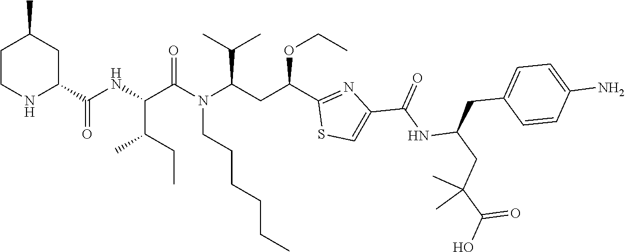 [SMILES: CCCCCCN(C(=O)[C@@H](NC(=O)[C@H]1C[C@H](C)CCN1)[C@@H](C)CC)[C@H](C[C@@H](OCC)c1nc(C(=O)N[C@@H](Cc2ccc(N)cc2)CC(C)(C)C(=O)O)cs1)C(C)C] |
| P22 | 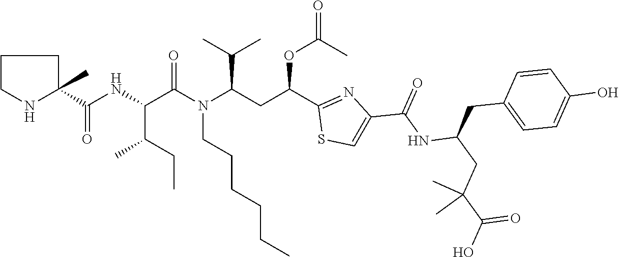 [SMILES: CCCCCCN(C(=O)[C@@H](NC(=O)[C@@]1(C)CCCN1)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| P25 | 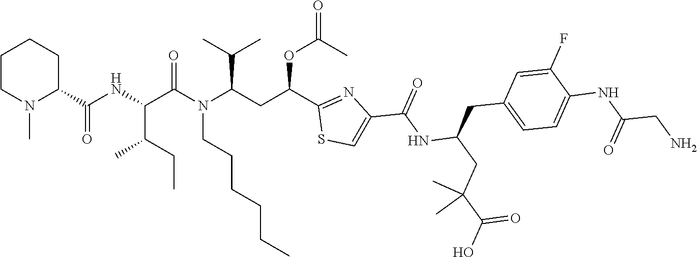 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P26 | 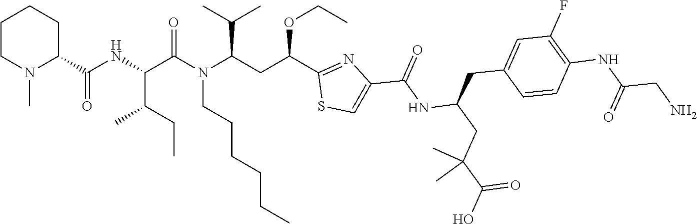 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)c(F)c2)CC(C)(C)C(=O)O)cs1)C(C)C] |
| P27 | 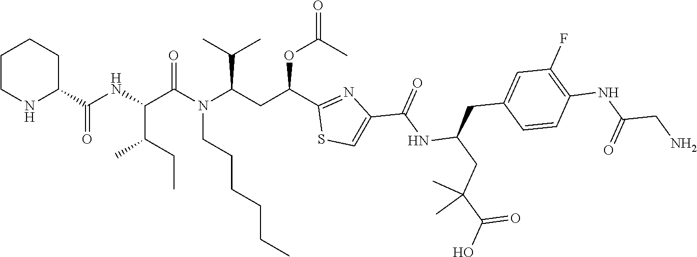 [SMILES: CCCCCCN(C(=O)[C@@H](NC(=O)[C@H]1CCCCN1)[C@@H](C)CC)[C@H](C[C@@H](OC(C)=O)c1nc(C(=O)N[C@@H](Cc2ccc(NC(=O)CN)c(F)c2)CC(C)(C)C(=O)O)cs1)C(C)C] |
| P28 | 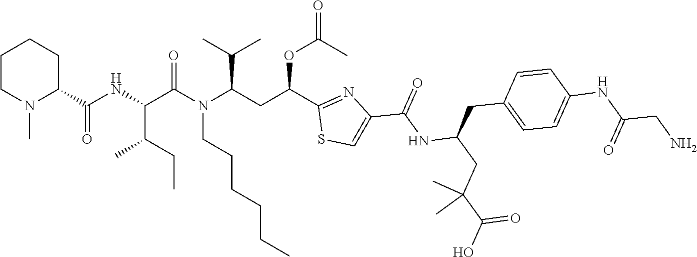 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |
| P31 | 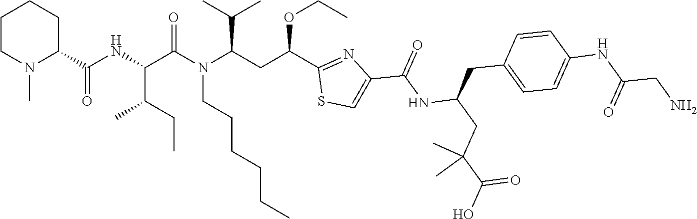 [SMILES: CCCCCCN(C(=O)[C@@H](NC(=O)[C@H]1CCCCN1C)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P32 | 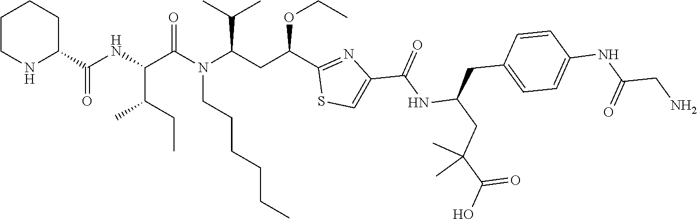 [SMILES: CCCCCCN(C(=O)[C@@H](NC(=O)[C@H]1CCCCN1)[C@@H](C)CC)[C@H](C[C@@H](OCC)c1nc(C(=O)N[C@@H](Cc2ccc(NC(=O)CN)cc2)CC(C)(C)C(=O)O)cs1)C(C)C] |
| P34 | 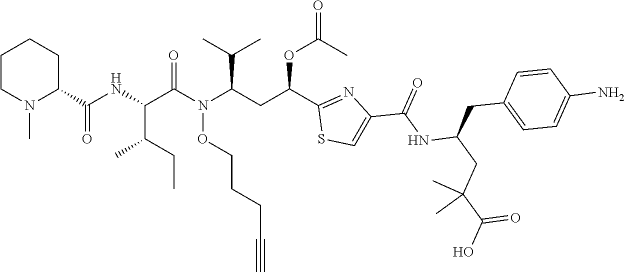 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P35 | 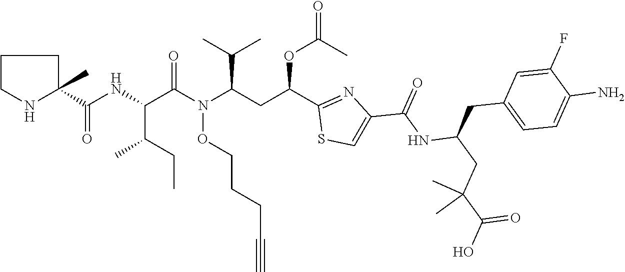 [SMILES: C#CCCCON(C(=O)[C@@H](NC(=O)[C@@]1(C)CCCN1)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| P36 | 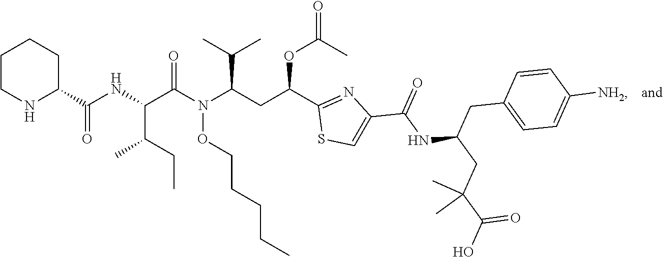 [SMILES: CCCCCON(C(=O)[C@@H](NC(=O)[C@H]1CCCCN1)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| P51 | 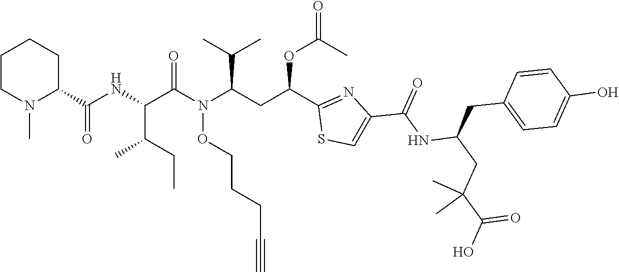 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| IVq | 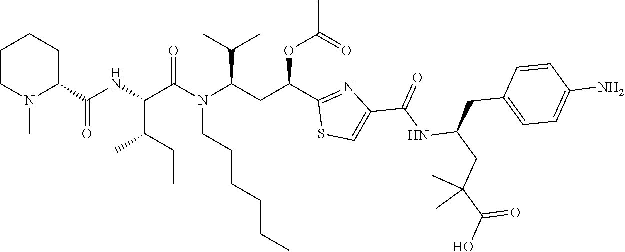 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IVu | 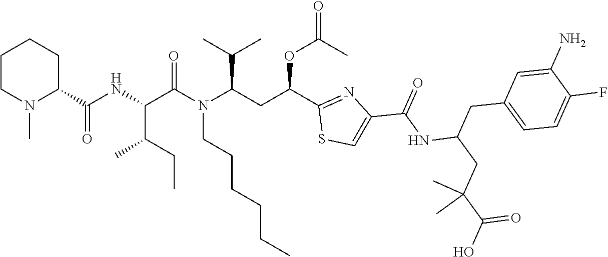 [SMILES: CCCCCCN(C(=O)[C@@H](NC(=O)[C@H]1CCCCN1C)[C@@H](C)CC)[C@H](C[C@@H](OC(C)=O)c1nc(C(=O)NC(Cc2ccc(F)c(N)c2)CC(C)(C)C(=O)O)cs1)C(C)C] |
| IVvA | 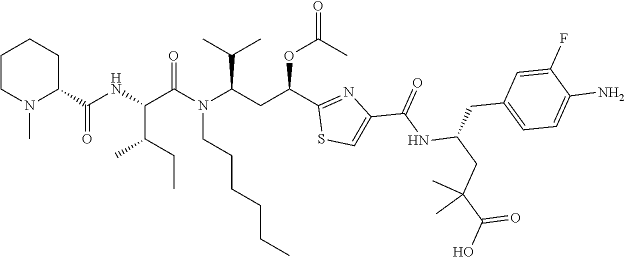 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@H](Cc2ccc(N)c(F)c2)CC(C)(C)C(=O)O)cs1)C(C)C] |
| IVvB | 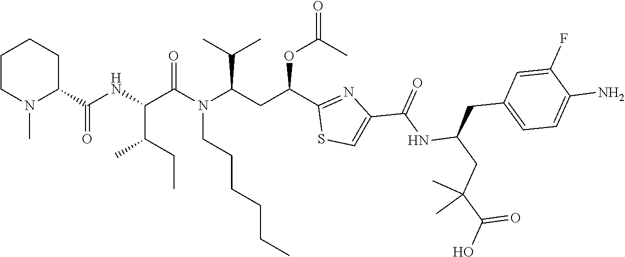 [SMILES: CCCCCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)c(F)c2)CC(C)(C)C(=O)O)cs1)C(C)C] |
| Vb | 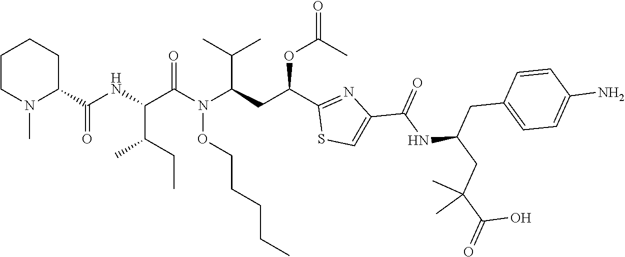 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| Ve | 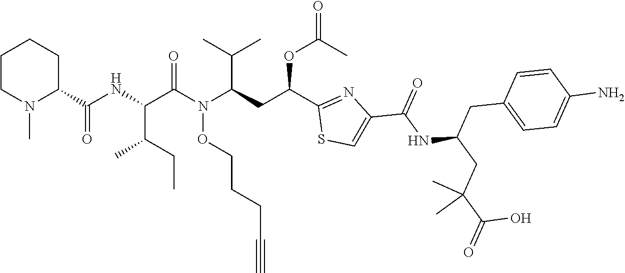 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)CC(C)(C)C(=O)O)cs1)C(C)C] |
| IX | 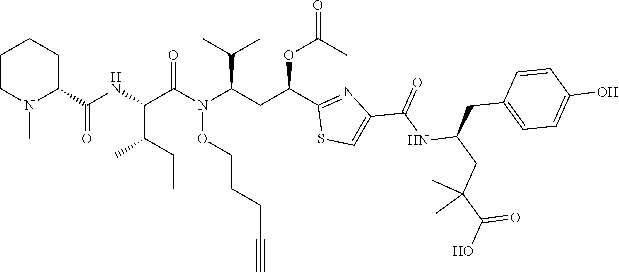 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] |
| X | 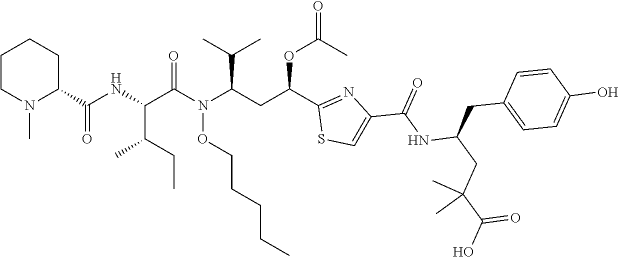 [SMILES: CCCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)CC(C)(C)C(=O)O)cs1)C(C)C] and |
| D-5a | 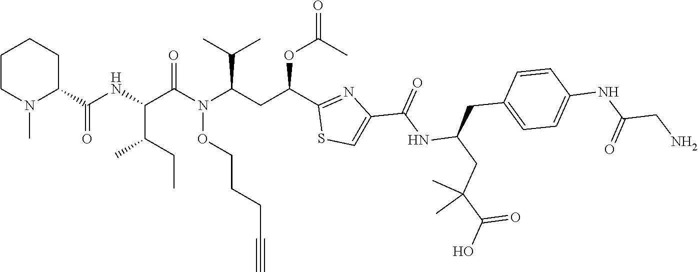 [SMILES: C#CCCCON(C(=O)[C@@H](NC(=O)[C@H]1CCCCN1C)[C@@H](C)CC)[C@H](C[C@@H](OC(C)=O)c1nc(C(=O)N[C@@H](Cc2ccc(NC(=O)CN)cc2)CC(C)(C)C(=O)O)cs1)C(C)C] |

72. The linker-payload of claim 71, having a Formula LPa, LPb, LPc, LPd, or LPe 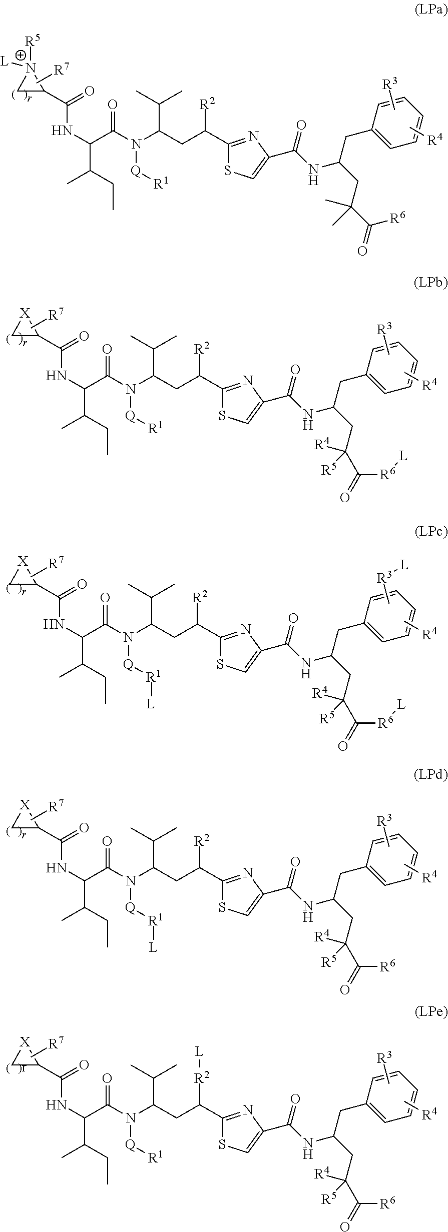
wherein L is a linker.

73. The linker-payload of claim 72, wherein R³ is —OH, —NH₂, —NH—(CH₂)₂OH, —NH—CH₂—C(O)—NH₂, —NH—CH₂—C(O)—OH, —NH—C(O)—CH₂NH₂, —NH—[(CH₂)₂OH]—C(O)—NH₂, or —NH—CH₂—(CH₂O)₂—(CH₂)₂—NH₂; or
R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or
R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂.

74. The linker-payload of claim 73, having the Formula LPa′, LPb′, LPc′, LPd′, or LPe′ 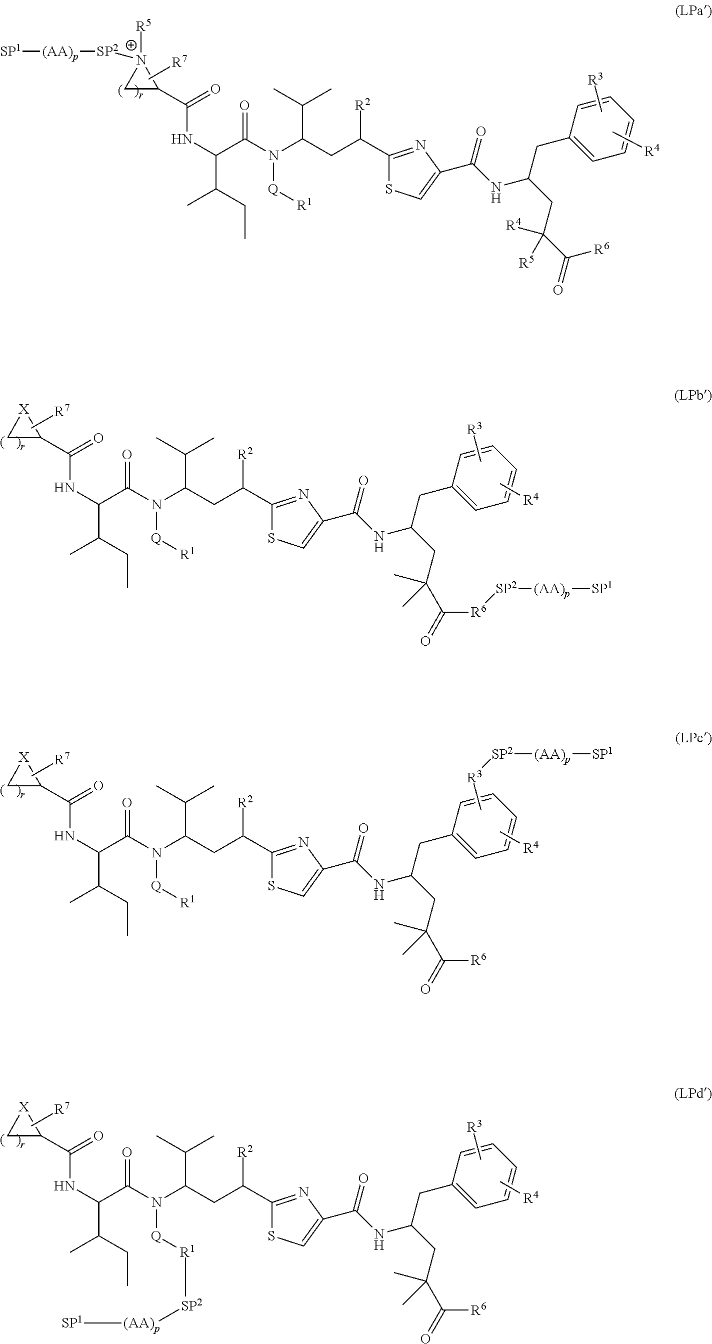 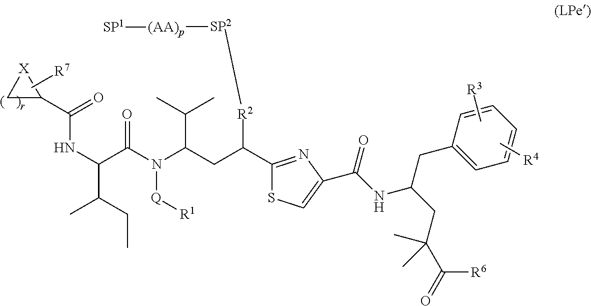
wherein SP¹ and SP², when present, are spacer groups;
each AA, when present, is a second amino acid residue; and
p is an integer from zero to ten.

75. The linker-payload of claim 74, wherein
the —SP²— spacer, when present, is 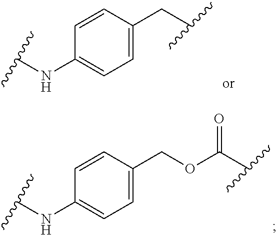 [SMILES: *Cc1ccc(N*)cc1 ; *Nc1ccc(COC(*)=O)cc1]
the second -(AA)ₚ- is 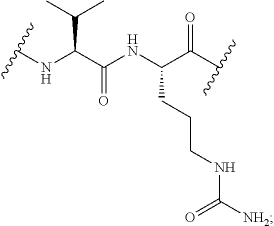 [SMILES: *N[C@H](C(=O)N[C@@H](CCCNC(N)=O)C(*)=O)C(C)C]
the —SP¹— spacer is 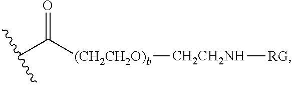
wherein RG is a reactive group; and
b is an integer from one to four.

76. The linker-payload of claim 74, wherein Q is —O—.

77. The linker-payload of claim 74, wherein
Q is —CH₂—;
X is —NR⁵,
R⁵ is —CH₃ or —(CH₂)₂—OH;
R¹ is —C₅ alkyl;
R⁶ is —OH;
R⁷ when present is —CH₃; and
r is three or four.

78. The linker-payload of claim 77, according to the structure of LPc′, or a pharmaceutically acceptable salt thereof.

79. The linker payload of claim 77, wherein R³ is —NH—(CH₂)₂OH, —NH—CH₂—C(O)—NH₂, —NH—C(O)—CH₂NH₂, —NH—CH₂—C(O)—NH—(CH₂CH₂O)₂—(CH₂)₂NH₂; or
R³ is —OH, —NH—(CH₂)₂OH, —NH—CH₂—C(O)—OH, or —N(CH₂CH₂OH)(C(O)CH₂NH₂) with a covalent bond to L from a terminal oxygen in any one of —OH, —NH—(CH₂)₂OH, —NH—CH₂—C(O)—OH, or —N(CH₂CH₂OH)(C(O)CH₂NH₂); or
R³ is —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂ with a covalent bond to L from a terminal nitrogen in any one of —NH₂, —NH—CH₂—C(O)—NH₂, —NH—C(O)—CH₂NH₂, —NH—[(CH₂)₂OH]—C(O)—NH₂, —NH—CH₂—(CH₂O)₂—(CH₂)₂—NH₂, —N(CH₂CH₂OH)(C(O)CH₂NH₂), or —NH—CH₂—C(O)—NH—(CH₂CH₂O)₂—(CH₂)₂NH₂; and
R⁴ is hydrogen or —F.

80. The linker-payload of claim 74, wherein
Q is —CH₂—;
X is —R⁵;
R⁵ is —CH₃;
R¹ is —C₅ alkyl;
R⁶ is —OH;
R⁷ when present is —CH₃; and
r is four.

81. The linker-payload of claim 80, according to the structure of LPe′, or a pharmaceutically acceptable salt thereof.

82. The linker payload of claim 81, wherein R² is —O—C(O)—NH—(CH₂CH₂O)₂—(CH₂)₂O—.

83. The linker-payload of claim 74, wherein Q is —O—.

84. The linker-payload of claim 74, wherein
Q is —CH₂—;
X is —NR⁵;
R¹ is —C₅ alkyl;
R⁶ is —OH;
R⁷ when present is —CH₃; and
r is three.

85. The linker-payload of claim 77, according to the structure of LPa′, or a pharmaceutically acceptable salt thereof.

86. The linker payload of claim 77, wherein R⁵ is —C(O)—CH₂—NH—.

87. The linker-payload of claim 74, wherein the linker-payload is selected from the group consisting of 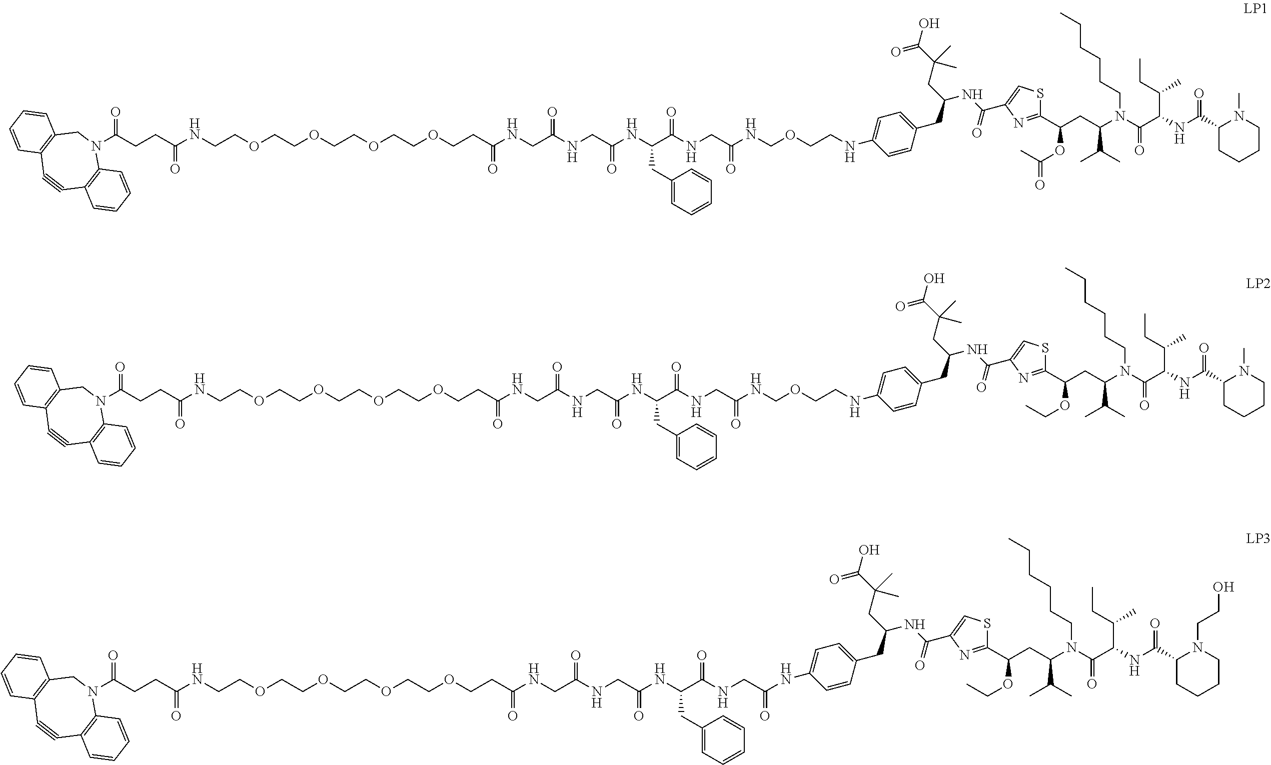 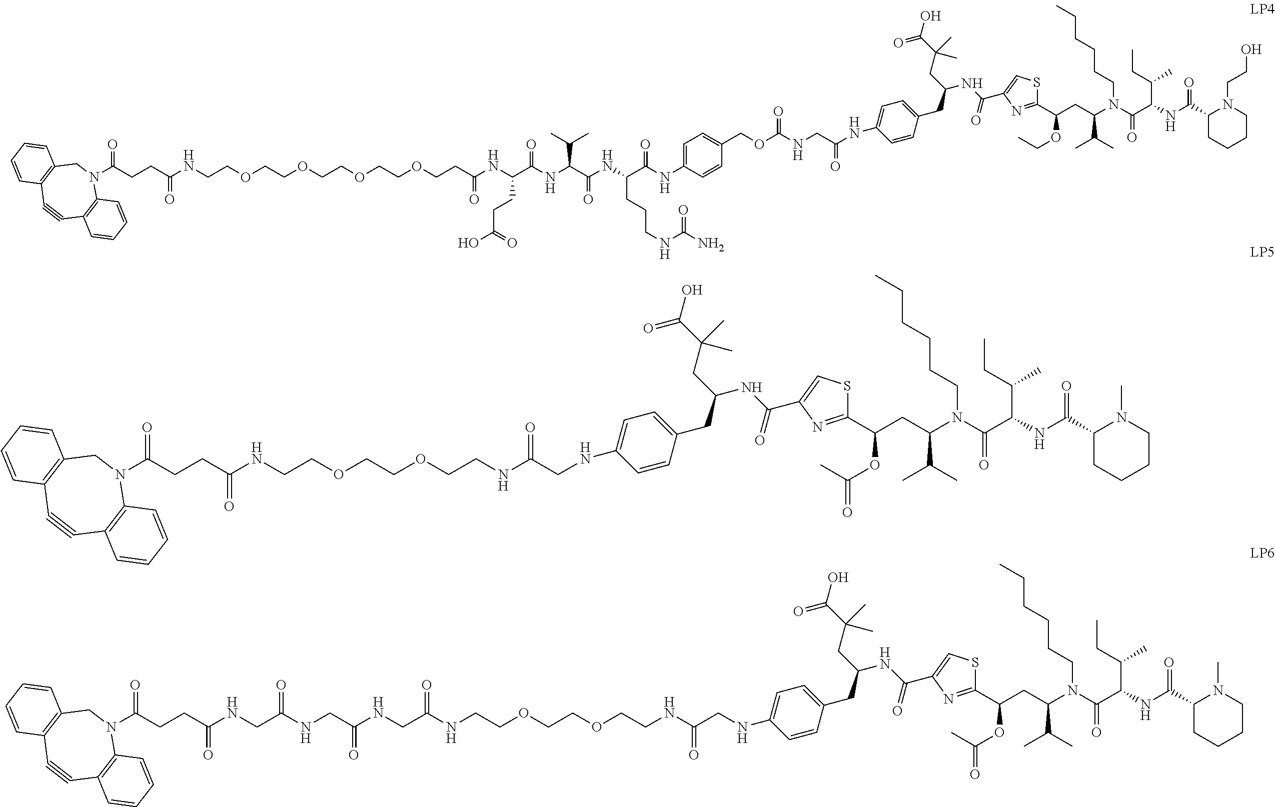 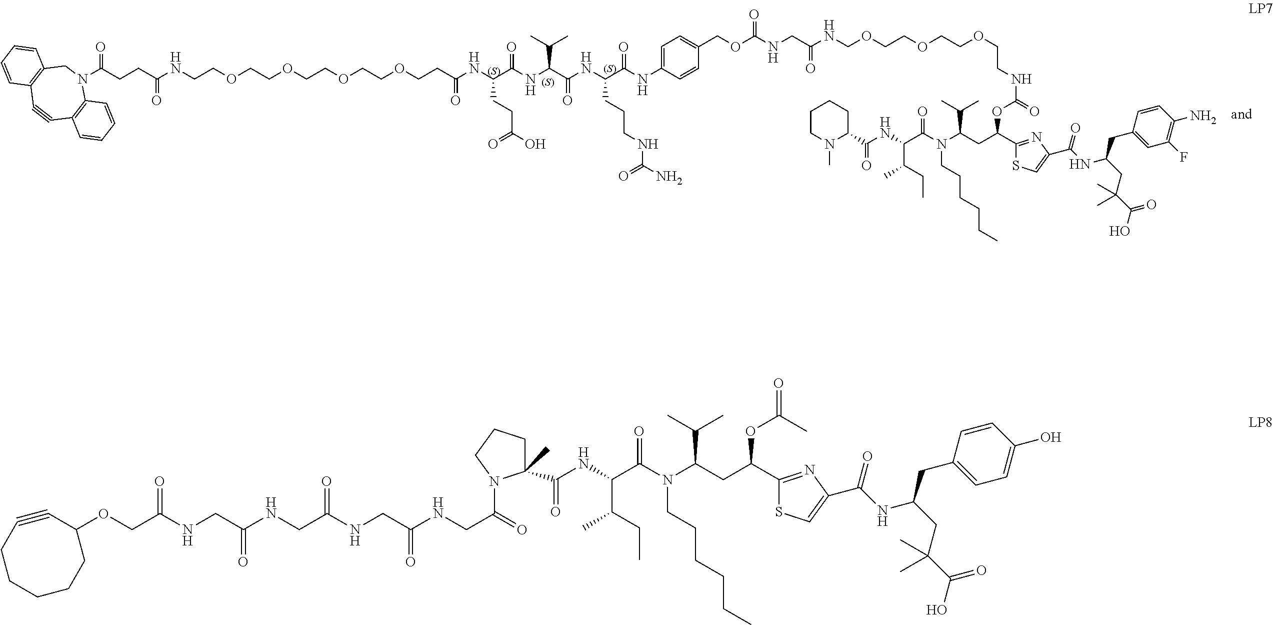
or
a pharmaceutically acceptable salt thereof.

88. A linker-payload selected from the group consisting of 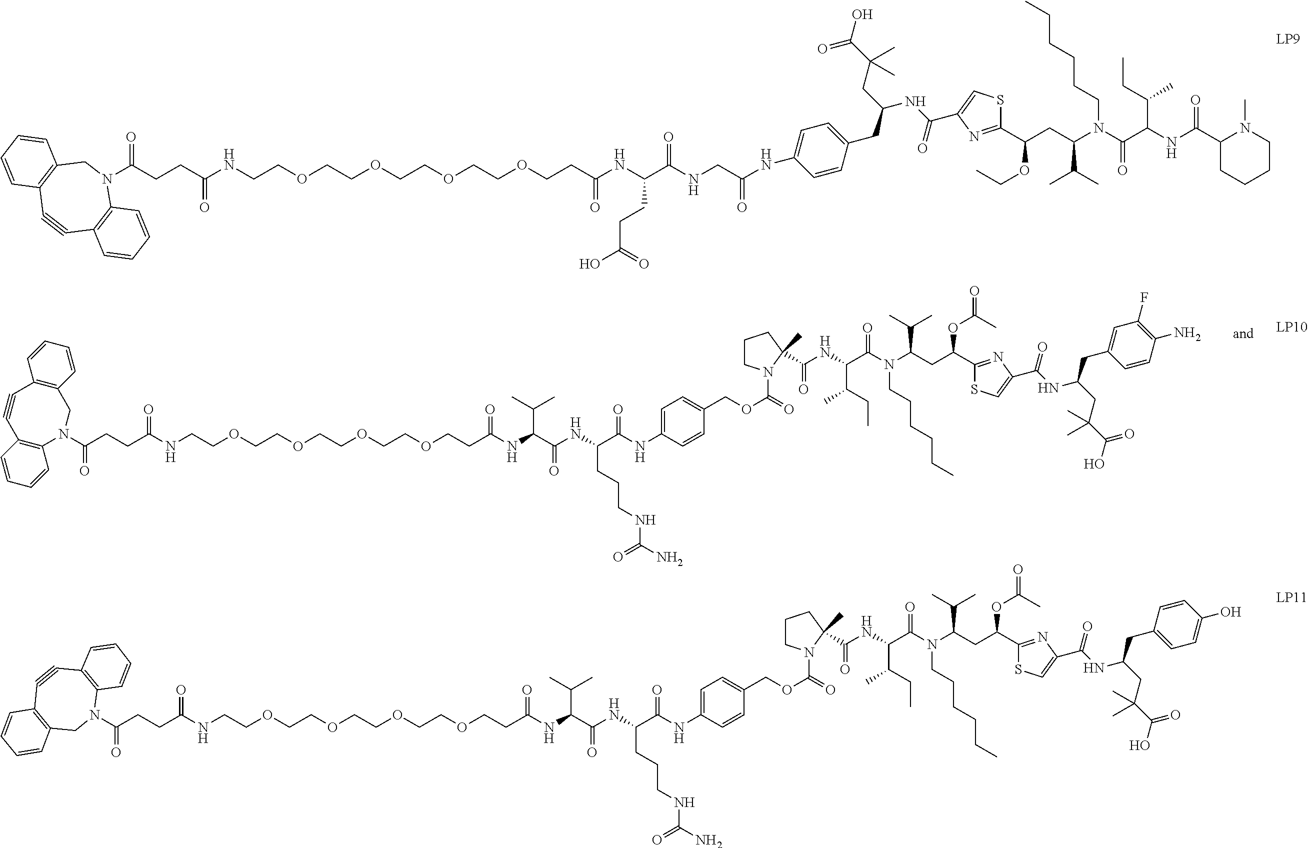
or
a pharmaceutically acceptable salt thereof.

89. A compound selected from the group consisting of 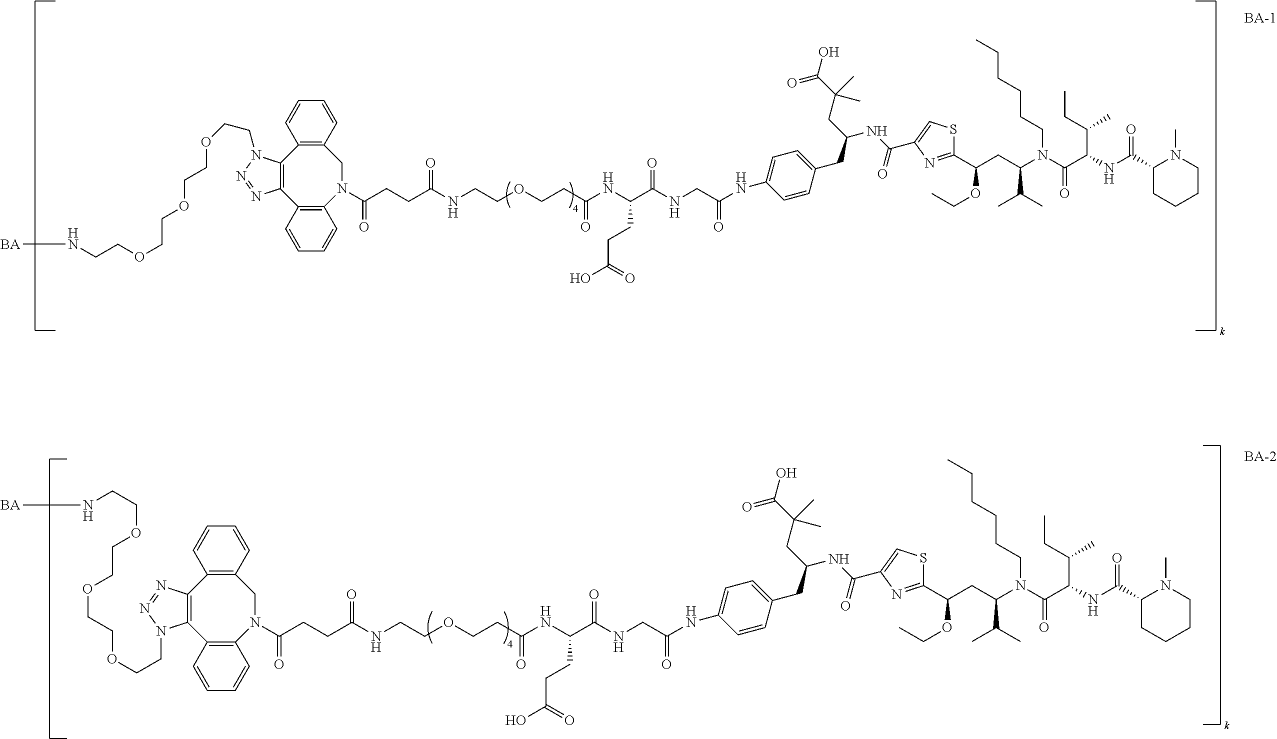 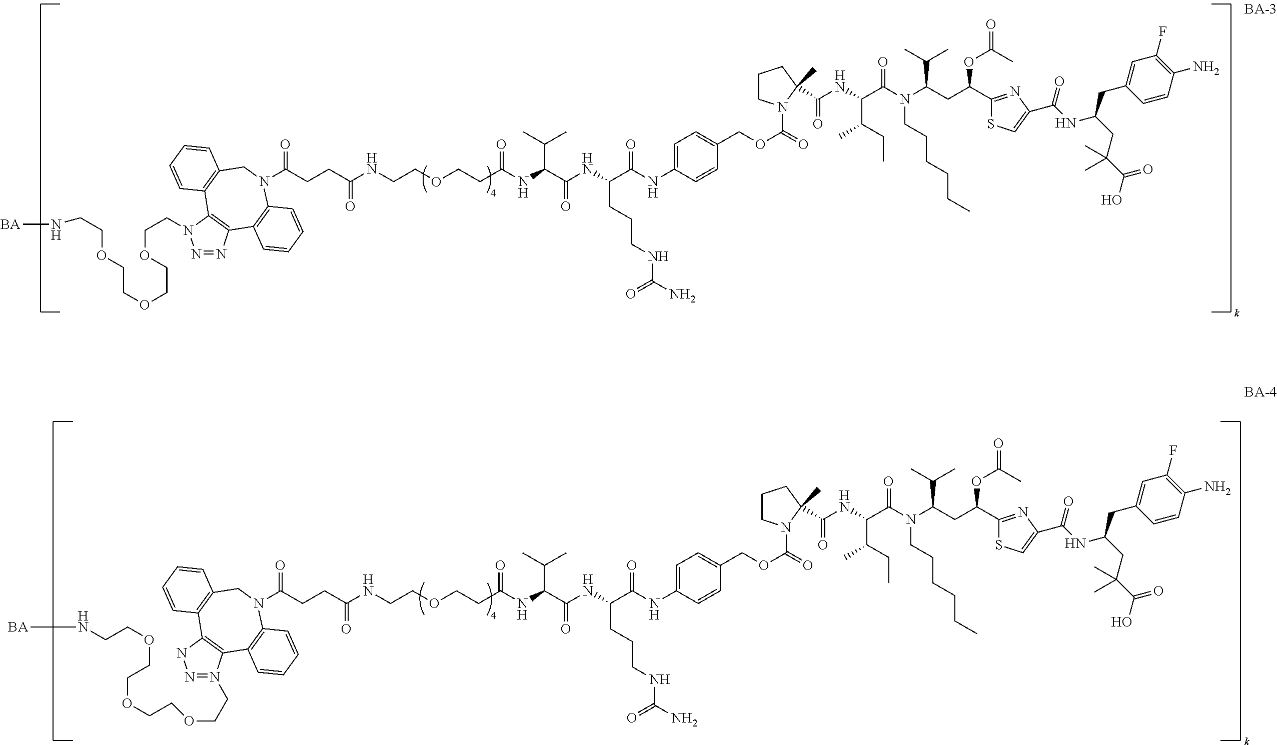 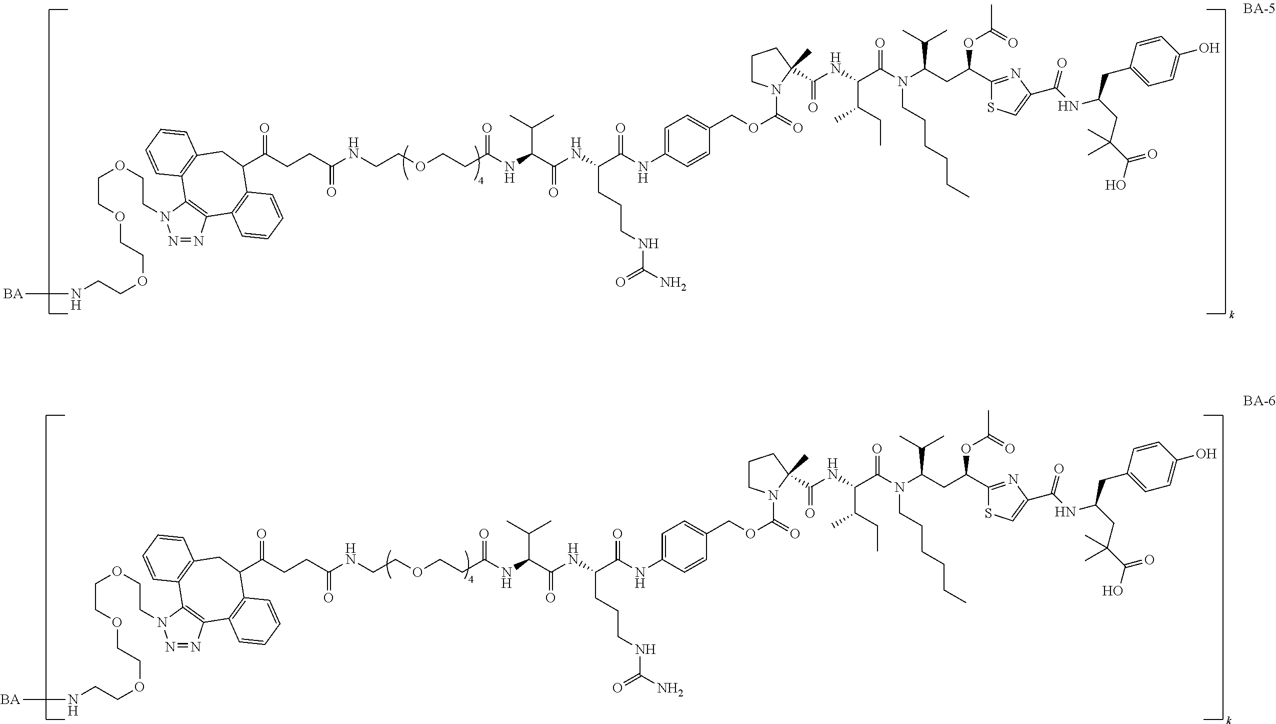
or a pharmaceutically acceptable salt thereof, wherein BA is a binding agent; and k is one, two, three, or four.


SEQUENCE LISTING (23 sequences)

SEQ ID NO: 1 - protein, 115 aa, synthetic construct
  REGION 1..115: note = Synthetic: H1H7814N
QVQLVESGGGVVQPGRSLRLSCVASGFTISSYGMNWVRQAPGKGLEWVAVISYDGGNKYSVDSVKGRFTISRDNSKNTLYLQMNSLRAEDSAVYYCARGRYFDLWGRGTLVTVSS

SEQ ID NO: 2 - protein, 8 aa, synthetic construct
  REGION 1..8: note = Synthetic: H1H7814N
GFTISSYG

SEQ ID NO: 3 - protein, 8 aa, synthetic construct
  REGION 1..8: note = Synthetic: H1H7814N
ISYDGGNK

SEQ ID NO: 4 - protein, 8 aa, synthetic construct
  REGION 1..8: note = Synthetic: H1H7814N
ARGRYFDL

SEQ ID NO: 5 - protein, 107 aa, synthetic construct
  REGION 1..107: note = Synthetic: H1H7814N
DIQMTQSPSTLSASVGDRVTITCRASQSISSWLAWYQQKPGRAPNLLISKASSLKSGVPSRFSGSGSGTEFTLTVSSLQPDDFATYYCQQYYSYSYTFGQGTKLEIK

SEQ ID NO: 6 - protein, 6 aa, synthetic construct
  REGION 1..6: note = Synthetic: H1H7814N
QSISSW

SEQ ID NO: 7 - skipped

SEQ ID NO: 8 - protein, 9 aa, synthetic construct
  REGION 1..9: note = Synthetic: H1H7814N
QQYYSYSYT

SEQ ID NO: 9 - protein, 115 aa, synthetic construct
  REGION 1..115: note = Synthetic: H1H6958N2
QVQLVESGGGVVQPGRSLRLSCGASGFTFRNYGMQWVRQGPGKGLEWVTLISFDGNDKYYADSVKGRFTISRDNSKNTLFLQMNSLRTEDTAVYYCARGGDFDYWGQGTLVTVSS

SEQ ID NO: 10 - protein, 8 aa, synthetic construct
  REGION 1..8: note = Synthetic: H1H6958N2
GFTFRNYG

SEQ ID NO: 11 - protein, 8 aa, synthetic construct
  REGION 1..8: note = Synthetic: H1H6958N2
ISFDGNDK

SEQ ID NO: 12 - protein, 8 aa, synthetic construct
  REGION 1..8: note = Synthetic: H1H6958N2
ARGGDFDY

SEQ ID NO: 13 - protein, 108 aa, synthetic construct
  REGION 1..108: note = Synthetic: H1H6958N2
DIQMTQSPSSLSASVGDRVTITCRASQDIRKDLGWYQQKPGKAPKRLIYAASSLHSGVPSRFSGSGSGTEFTLTISSLQPEDFATYYCLQHNSYPMYTFGQGTKLEIK

SEQ ID NO: 14 - protein, 6 aa, synthetic construct
  REGION 1..6: note = Synthetic: H1H6958N2
QDIRKD

SEQ ID NO: 15 - skipped

SEQ ID NO: 16 - protein, 10 aa, synthetic construct
  REGION 1..10: note = Synthetic: H1H6958N2
LQHNSYPMYT

SEQ ID NO: 17 - protein, 267 aa, synthetic construct
  REGION 1..267: note = Synthetic: hPRLR ecto-MMH
MHRPRRRGTRPPPLALLAALLLAARGADAQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDEQKLISEEDLGGEQKLISEEDLHHHHHH

SEQ ID NO: 18 - protein, 5 aa, synthetic construct
  REGION 1..5: note = Synthetic: linker
GGGGS

SEQ ID NO: 19 - protein, 5 aa, synthetic construct
  REGION 1..5: note = Synthetic: linker
GGGGG

SEQ ID NO: 20 - protein, 5 aa, synthetic construct
  REGION 1..5: note = Synthetic: linker
GGGGK

SEQ ID NO: 21 - protein, 4 aa, synthetic construct
  REGION 1..4: note = Synthetic: linker
GFGG

SEQ ID NO: 22 - protein, 4 aa, synthetic construct
  REGION 1..4: note = Synthetic: linker
GGGG

SEQ ID NO: 23 - protein, 4 aa, synthetic construct
  REGION 1..4: note = Synthetic: linker
GGFG